(19) United States
(12) Patent Application Publication
(10) Pub. No.: US 2026/0042781 A1
(43) Pub. Date: Feb. 12, 2026

(54) Bicyclic Ureas As Kinase Inhibitors

(71) Applicant: Incyte Corporation, Wilmington, DE (US)

(72) Inventors: Xin Li, Claymont, DE (US); Seoyoung Cho, Wilmington, DE (US); Hang Thi Dang, Wilmington, DE (US); Dipendu Das, Wilmington, DE (US); Brent Douty, Fallowfiled, PA (US); Michael Liang, Wilmington, DE (US); Kai Liu, Chadds Ford, PA (US); Minh Nguyen, Claymont, DE (US); Hunter Sims, Wilmington, DE (US); Evan Styduhar, Media, PA (US); Robert Swyka, Wilmington, DE (US); Oleg Vechorkin, Wilmington, DE (US); Darius Vrubliauskas, Wilmington, DE (US); Anlai Wang, Wilmington, DE (US); Hai Fen Ye, Newark, DE (US); Eddy W. Yue, Landenberg, PA (US); Ke Zhang, Garnet Valley, PA (US); Peng Zhao, Newark, DE (US)

(21) Appl. No.: 19/277,819

(22) Filed: Jul. 23, 2025

Related U.S. Application Data
(60) Provisional application No. 63/675,113, filed on Jul. 24, 2024.
(60) Provisional application No. 63/738,971, filed on Dec. 26, 2024.
(60) Provisional application No. 63/783,499, filed on Apr. 4, 2025.
(60) Provisional application No. 63/789,774, filed on Apr. 16, 2025.

(51) Int. Cl. C07D 519/00; A61K 31/437; A61K 31/444; A61K 31/5025; A61K 31/5377; A61P 7/00; C07B 59/00

(52) U.S. Cl. CPC C07D 519/00; A61K 31/437; A61K 31/444; A61K 31/5025; A61K 31/5377; A61P 7/00; C07B 59/002

(57) ABSTRACT

The present application provides bicyclic urea compounds that modulate the activity of JAK2, which are useful in the treatment of various diseases, including cancer.

### SEQUENCE LISTING

This application contains a Sequence Listing that has been submitted electronically as an XML file named “20443-0861001_SL_ST26.XML.” The XML file, created on Jul. 21, 2025, is 2,281 bytes in size. The material in the XML file is hereby incorporated by reference in its entirety.

### TECHNICAL FIELD

The present invention provides bicyclic urea compounds that modulate the activity of the JAK2 and are useful in the treatment of diseases related to JAK2, including cancer.

### BACKGROUND

Janus kinase (JAK) 2 plays pivotal roles in signaling by several cytokine receptors. The mutant JAK2 V617F, located at pseudokinase (JH2) domain, is the most common molecular event associated with myeloproliferative neoplasms (MPNs). Current JAK2 small molecule inhibitors used to treat MPNs are designed to target the JAK2 kinase (JH1) domain. Thus, selective targeting of the JAK2 V617F mutant over the JAK2 kinase (JH1) domain may be useful for treating various pathologies, while sparing essential JAK2 functions. This application is directed to this need and others.

### SUMMARY

The present invention relates to, inter alia, compounds of Formula Ia:

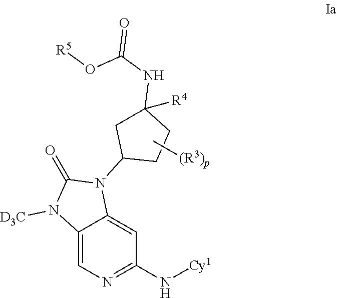

or pharmaceutically acceptable salts thereof, wherein constituent members are defined herein.

The present invention further provides compounds of Formula I:

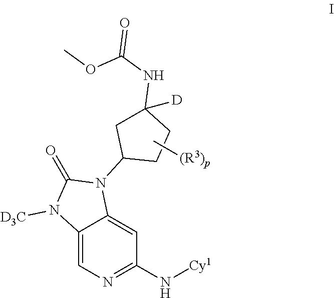

or pharmaceutically acceptable salts thereof, wherein constituent members are defined herein.

The present invention further provides pharmaceutical compositions comprising a compound of Formula Ia or Formula I, or a pharmaceutically acceptable salt thereof, and a pharmaceutically acceptable carrier.

The present invention further provides methods of inhibiting an activity of the V617F variant of JAK2 kinase comprising contacting the kinase with a compound of Formula Ia or Formula I, or a pharmaceutically acceptable salt thereof.

The present invention further provides methods of treating a disease or a disorder associated with expression or activity of the V617F variant of JAK2 kinase in a patient by administering to a patient a therapeutically effective amount of a compound of Formula Ia or Formula I, or a pharmaceutically acceptable salt thereof.

The present invention further provides a compound of Formula Ia or Formula I, or a pharmaceutically acceptable salt thereof, for use in any of the methods described herein.

The present invention further provides use of a compound of Formula Ia or Formula I, or a pharmaceutically acceptable salt thereof, for the preparation of a medicament for use in any of the methods described herein.

### DETAILED DESCRIPTION

The present application provides a compound of Formula Ia:

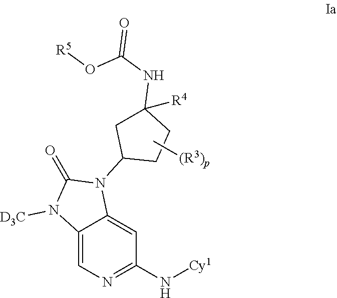

or a pharmaceutically acceptable salt thereof, wherein:
   Cy¹ is selected from

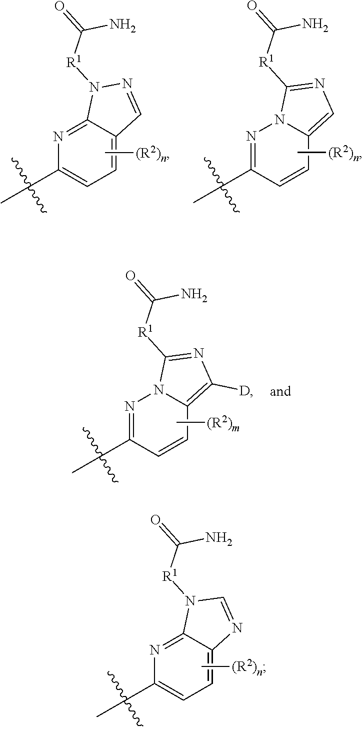

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, oxo, C₁₋₃ alkyl, C₁₋₃ haloalkyl, C₁₋₃ alkoxy, C₁₋₃ haloalkoxy, C₂₋₃ alkynyl, C₃₋₅ cycloalkyl, CN, OH, C(O)OH, and NH₂;
   each R² is independently selected from halo, oxo, C₁₋₆ alkyl, C₁₋₆haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, CN, OR^{a21}, C(O)NR^{c21}R^{d21}, OC(O)NR^{c21}R^{d21}, NR^{c21}R^{d21}, NR^{c21}C(O)R^{d21}, NR^{c21}C(O)OR^{d21}, NR^{c21}C(O)NR^{c21}R^{d21}, NR^{c21}S(O)₂R^{d21}, NR^{c21}S(O)(═NR^{e21})R^{b21}, NR^{c21}S(O)₂NR^{c21}R^{d21}, S(O)₂R^{d21}, S(O)₂NR^{c21}R^{d21} OS(O)(═NR^{e21})R^{b21}, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
   each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, or 4 independently selected R^{2A} substituents;
   or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
   each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, or 4 independently selected R^{2A} substituents;
   each R^{e21} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
   each R^{2A} is independently selected from H, OH, halo, oxo, CN, C(O)OH, NH₂, NO₂, SF₅, C₁₋₆ alkyl, C₁₋₆ alkoxy, C₁₋₆haloalkoxy, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, or 4 independently selected R^{M} substituents;
   each R³ is independently selected from halo, oxo, C₁₋₃ alkyl, C₁₋₃ haloalkyl, C₁₋₃ alkoxy, C₁₋₃ haloalkoxy, CN, OH, C(O)OH, and NH₂;
   R⁴ is H or D;
   R⁵ is methyl or trideuteromethyl;
   m is 0, 1, or 2;
   n is 0, 1, 2, or 3;
   p is 0, 1, or 2; and
   each R^{M} is independently selected from OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

The present application provides a compound of Formula Ia:

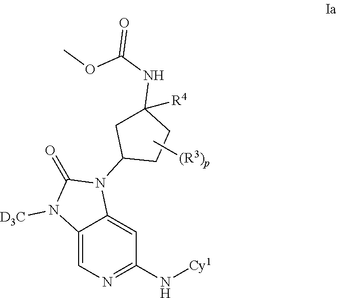

or a pharmaceutically acceptable salt thereof, wherein:
   Cy¹ is selected from

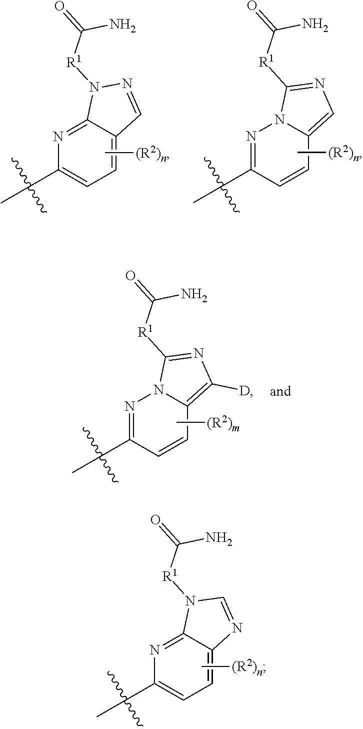

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, oxo, C₁₋₃ alkyl, C₁₋₃ haloalkyl, C₁₋₃ alkoxy, C₁₋₃ haloalkoxy, C₂₋₃ alkynyl, C₃₋₅ cycloalkyl, CN, OH, C(O)OH, and NH₂;
each R² is independently selected from halo, oxo, C₁₋₆ alkyl, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, CN, OR^{a21}, C(O)NR^{c21}R^{d21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)₂R^{b21}, S(O)₂NR^{c21}R^{d21}, OS(O)(═NR^{e21})R^{b21}, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, or 4 independently selected R^{2A} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, or 4 independently selected R^{2A} substituents;
each R^{e21} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is independently selected from H, OH, halo, oxo, CN, C(O)OH, NH₂, NO₂, SF₅, C₁₋₆ alkyl, C₁₋₆alkoxy, C₁₋₆haloalkoxy, C₁₋₆haloalkyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, or 4 independently selected R^{M} substituents;
each R³ is independently selected from halo, oxo, C₁₋₃ alkyl, C₁₋₃ haloalkyl, C₁₋₃ alkoxy, C₁₋₃ haloalkoxy, CN, OH, C(O)OH, and NH₂;
R⁴ is H or D;
m is 0, 1, or 2;
n is 0, 1, 2, or 3;
p is 0, 1, or 2; and
each R^{M} is independently selected from OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments, R⁴ is H.

In some embodiments, R⁴ is D.

The present application further provides a compound of Formula I:

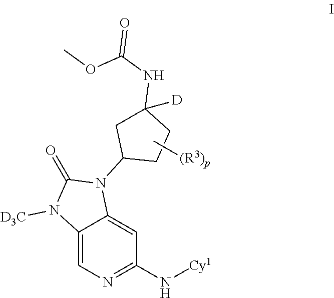

or a pharmaceutically acceptable salt thereof, wherein:
   Cy¹ is selected from and

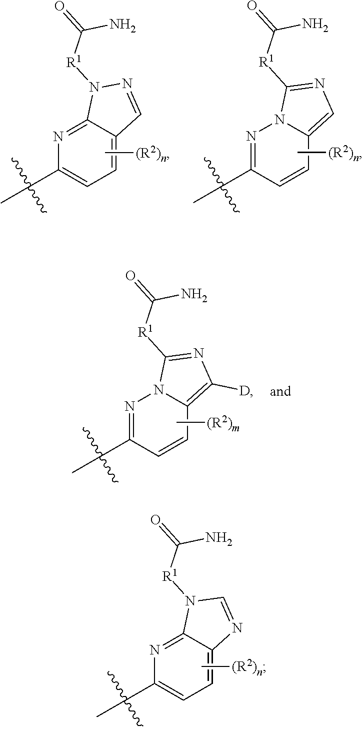

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, oxo, C₁₋₃ alkyl, C₁₋₃ haloalkyl, C₁₋₃ alkoxy, C₁₋₃ haloalkoxy, CN, OH, C(O)OH, and NH₂;
each R² is independently selected from halo, oxo, C₁₋₆ alkyl, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, CN, OR^{a21}, C(O)NR^{c21}R^{d21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)₂R^{b21}, S(O)₂NR^{c21}R^{d21}, OS(O)(═NR^{e21})R^{b21}, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
   each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, or 4 independently selected R^{2A} substituents;
   or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
   each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{d21} are each optionally substituted with 1, 2, 3, or 4 independently selected R^{2A} substituents;
   each R^{e21} is independently selected from H, OH, CN, C₁₋₆ alkyl, C₁₋₆ alkoxy, C₁₋₆ haloalkyl, C₁₋₆haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is independently selected from H, OH, halo, oxo, CN, C(O)OH, NH₂, NO₂, SF₅, C₁₋₆ alkyl, C₁₋₆alkoxy, C₁₋₆haloalkoxy, C₁₋₆haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, or 4 independently selected R^{M} substituents;
   each R³ is independently selected from halo, oxo, C₁₋₃ alkyl, C₁₋₃ haloalkyl, C₁₋₃ alkoxy, C₁₋₃ haloalkoxy, CN, OH, C(O)OH, and NH₂;
   m is 0, 1, or 2;
   n is 0, 1, 2, or 3;
   p is 0, 1, or 2; and
   each R^{M} is independently selected from OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments, n is 0, 1, or 2.

In some embodiments, n is 0 or 1.

In some embodiments, n is 1 or 2.

In some embodiments, n is 0.

In some embodiments, n is 1.

In some embodiments, n is 2.

In some embodiments, m is 0 or 1.

In some embodiments, m is 0.

In some embodiments, Cy¹ is

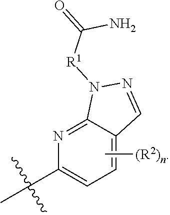

In some embodiments, Cy¹ is

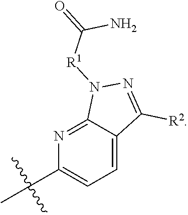

In some embodiments, Cy¹ is

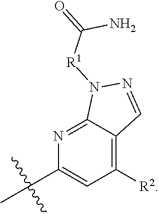

In some embodiments, Cy¹ is

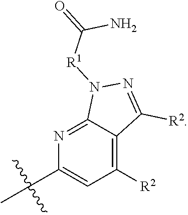

In some embodiments, Cy¹ is

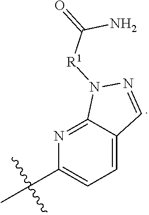

In some embodiments, Cy¹ is

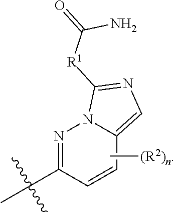

In some embodiments, Cy¹ is

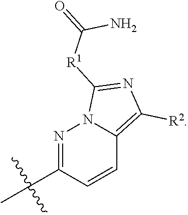

In some embodiments, Cy¹ is

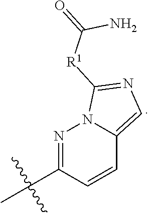

In some embodiments, Cy¹ is

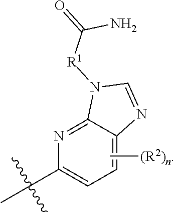

In some embodiments, Cy¹ is

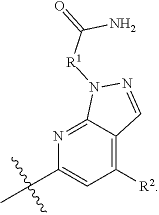

In some embodiments, Cy¹ is

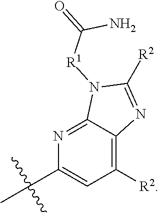

In some embodiments, Cy¹ is

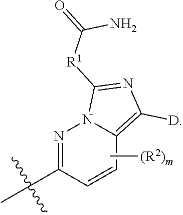

In some embodiments, Cy¹ is

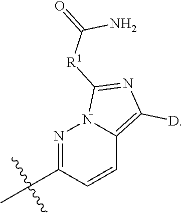

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, oxo, C₁₋₃ alkyl, C₁₋₃ haloalkyl, C₁₋₃ alkoxy, C₁₋₃ haloalkoxy, C₂₋₄ alkynyl, C₃₋₅ cycloalkyl, CN, OH, C(O)OH, and NH₂.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, oxo, C₁₋₃ alkyl, C₁₋₃ haloalkyl, C₁₋₃ alkoxy, C₁₋₃ haloalkoxy, CN, OH, C(O)OH, and NH₂.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1 or 2 R^{1A} substituents independently selected from halo, oxo, C₁₋₃ alkyl, C₁₋₃ haloalkyl, C₁₋₃ alkoxy, C₁₋₃ haloalkoxy, C₂₋₄ alkynyl, C₃₋₅ cycloalkyl, CN, OH, C(O)OH, and NH₂.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1 or 2 R^{1A} substituents independently selected from halo, oxo, C₁₋₃ alkyl, C₁₋₃ haloalkyl, C₁₋₃ alkoxy, C₁₋₃ haloalkoxy, CN, OH, C(O)OH, and NH₂.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, C₁₋₃ alkyl, C₁₋₃ haloalkyl, C₁₋₃ alkoxy, C₂₋₄ alkynyl, and C₃₋₅ cycloalkyl.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1 or 2 R^{1A} substituents independently selected from halo, C₁₋₃ alkyl, C₁₋₃ haloalkyl, C₁₋₃ alkoxy, C₂₋₄ alkynyl, and C₃₋₅ cycloalkyl.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, C₁₋₃ alkyl, C₁₋₃ alkoxy, C₂₋₄ alkynyl, and C₃₋₅ cycloalkyl.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, C₁₋₃ alkyl, and C₁₋₃ alkoxy.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1 or 2 R^{1A} substituents independently selected from halo, C₁₋₃ alkyl, C₁₋₃ alkoxy, C₂₋₄ alkynyl, and C₃₋₅ cycloalkyl.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1 or 2 R^{1A} substituents independently selected from halo, C₁₋₃ alkyl, and C₁₋₃ alkoxy.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from fluoro, methyl, ethyl, isopropyl, difluoromethyl, trifluoromethyl, ethynyl, methoxy, and cyclopropyl.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from fluoro, methyl, and methoxy.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1 or 2 R^{1A} substituents independently selected from fluoro, methyl, ethyl, isopropyl, difluoromethyl, trifluoromethyl, ethynyl, methoxy, and cyclopropyl.

In some embodiments, R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1 or 2 R^{A} substituents independently selected from fluoro, methyl, and methoxy.

In some embodiments, R¹ is selected from,

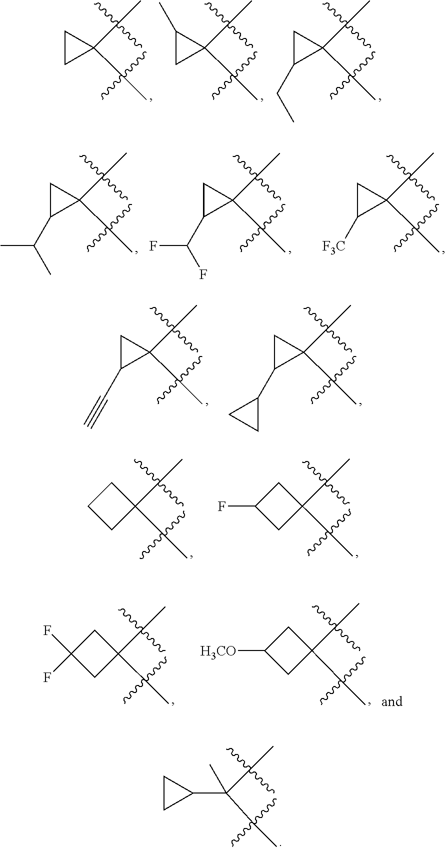

In some embodiments, R¹ is selected from

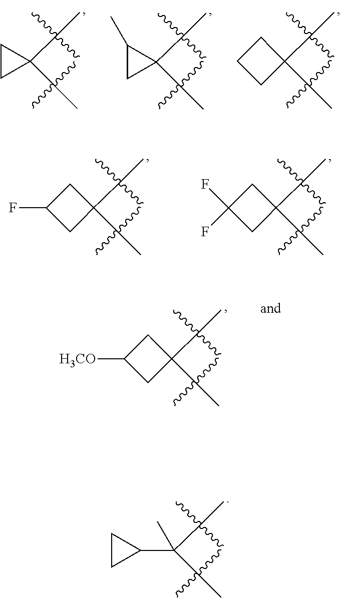

In some embodiments, R¹ is selected from

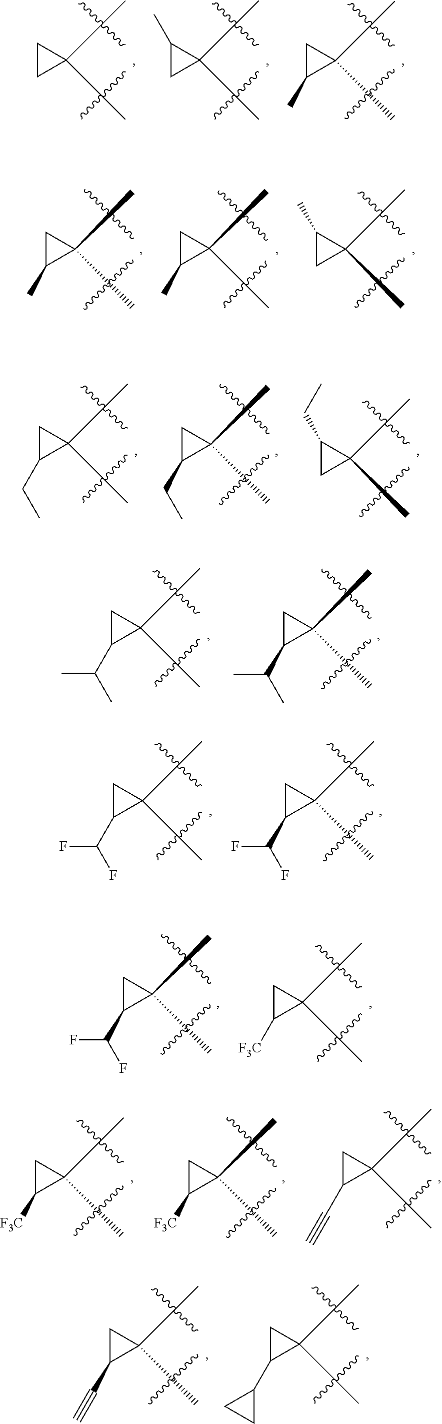

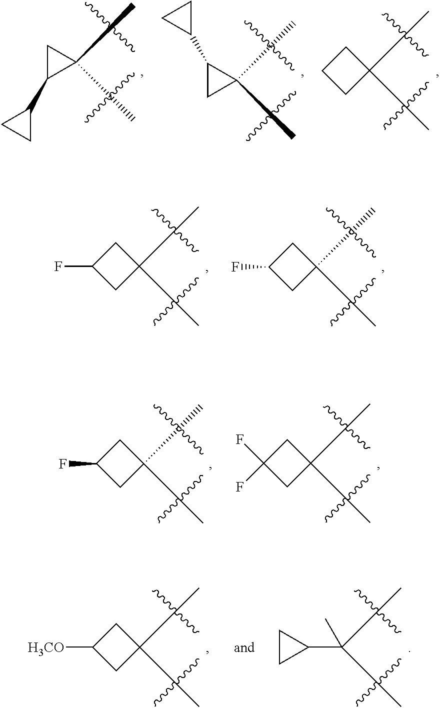

In some embodiments R¹ is selected from

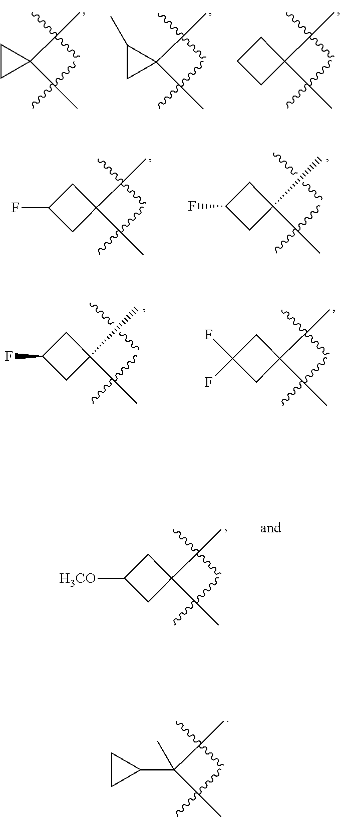

In some embodiments, R¹ is

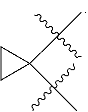 [SMILES: *C1(*)CC1]

In some embodiments, R¹ is

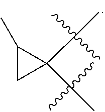 [SMILES: *C1(*)CC1C]

In some embodiments, R¹ is

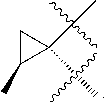

In some embodiments R¹ is

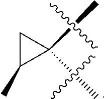

In some embodiments, R¹ is

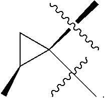

In some embodiments, R¹ is

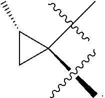

In some embodiments, R¹ is

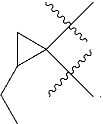 [SMILES: *C1(*)CC1CC ; "."]

In some embodiments, R¹ is

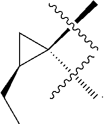

In some embodiments, R¹ is

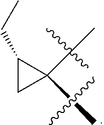

In some embodiments, R¹ is

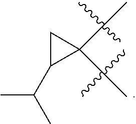 [SMILES: *C1(*)CC1C(C)C ; "."]

In some embodiments, R¹ is

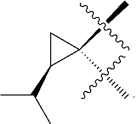

In some embodiments, R¹ is

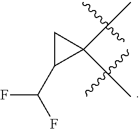 [SMILES: *C1(*)CC1C(F)F ; "."]

In some embodiments, R¹ is

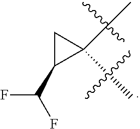

In some embodiments, R¹ is

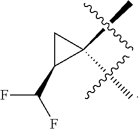

In some embodiments, R¹ is

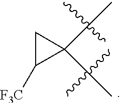 [SMILES: *C1(*)CC1C(F)(F)F ; "."]

In some embodiments, R¹ is

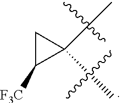

In some embodiments, R¹ is

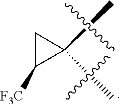

In some embodiments, R¹ is

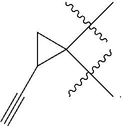 [SMILES: *C1(*)CC1C#C ; "."]

In some embodiments, R¹ is

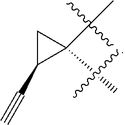

In some embodiments, R¹ is

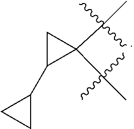 [SMILES: *C1(*)CC1C1CC1]

In some embodiments, R¹ is

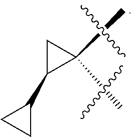

In some embodiments, R¹ is

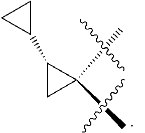

In some embodiments, R¹ is

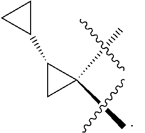

In some embodiments, R¹ is

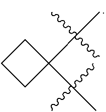 [SMILES: *C1(*)CCC1]

In some embodiments, R¹ is

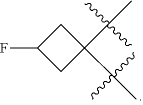 [SMILES: *C1(*)CC(F)C1]

In some embodiments, R¹ is

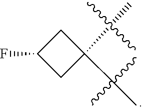

In some embodiments, R¹ is

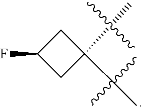

In some embodiments, R¹ is

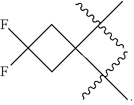 [SMILES: *C1(*)CC(F)(F)C1]

In some embodiments, R¹ is

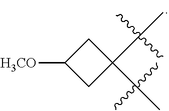 [SMILES: *C1(*)CC(OC)C1]

In some embodiments, R¹ is

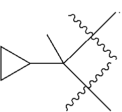 [SMILES: *C(*)(C)C1CC1]

In some embodiments, each R² is independently selected from C₁₋₆ alkyl, C₁₋₆ haloalkyl, C₃₋₁₀ cycloalkyl, 5-10 membered heteroaryl, 4-10 membered heterocycloalkyl, OR^{a21}, NR^{c21}R^{d21}, C(O)NR^{c21}R^{d21}, and NR^{c21}C(O)OR^{a21}, wherein the C₁₋₆ alkyl, C₃₋₁₀ cycloalkyl, 5-10 membered heteroaryl, and 4-10 membered heterocycloalkyl of R² are each optionally substituted with 1, 2, 3, or 4 independently selected R^{2A} substituents.

In some embodiments, each R² is independently selected from C₁₋₆ alkyl, C₁₋₆ haloalkyl, C₃₋₁₀ cycloalkyl, 5-10 membered heteroaryl, 4-10 membered heterocycloalkyl, OR^{a21}, C(O)NR^{c21}R^{d21}, and NR^{c21}C(O)OR^{a21}, wherein the C₁₋₆ alkyl, C₃₋₁₀ cycloalkyl, 5-10 membered heteroaryl, and 4-10 membered heterocycloalkyl of R² are each optionally substituted with 1, 2, 3, or 4 independently selected R^{2A} substituents.

In some embodiments, each R² is independently selected from C₁₋₆ alkyl, C₁₋₆ haloalkyl, C₃₋₁₀ cycloalkyl, and 4-10 membered heterocycloalkyl, wherein the C₁₋₆ alkyl, C₃₋₁₀ cycloalkyl, and 4-10 membered heterocycloalkyl of R² are each optionally substituted with 1, 2, 3, or 4 independently selected R^{2A} substituents.

In some embodiments, each R² is independently selected from C₁₋₆ alkyl, C₁₋₆ haloalkyl, C₃₋₇ cycloalkyl, 5-6 membered heteroaryl, 4-7 membered heterocycloalkyl, OR^{a21}, NR^{c21}R^{d21}, C(O)NR^{c21}R^{d21}, and NR^{c21}C(O)OR^{a21}, wherein the C₁₋₆ alkyl, C₃₋₇ cycloalkyl, 5-6 membered heteroaryl, and 4-7 membered heterocycloalkyl of R² are each optionally substituted with 1, 2, 3, or 4 independently selected R^{2A} substituents

In some embodiments, each R² is independently selected from C₁₋₆ alkyl, C₁₋₆ haloalkyl, C₃₋₇ cycloalkyl, 5-6 membered heteroaryl, 4-7 membered heterocycloalkyl, OR^{a21}, C(O)NR^{c21}R^{d21}, and NR^{c21}C(O)OR^{a21}, wherein the C₁₋₆ alkyl, C₃₋₇ cycloalkyl, 5-6 membered heteroaryl, and 4-7 membered heterocycloalkyl of R² are each optionally substituted with 1, 2, 3, or 4 independently selected R^{2A} substituents.

In some embodiments, each R² is independently selected from C₁₋₆ alkyl, C₁₋₆ haloalkyl, C₃₋₇ cycloalkyl, and 4-7 membered heterocycloalkyl, wherein the C₁₋₆ alkyl, C₃₋₇ cycloalkyl, and 4-7 membered heterocycloalkyl of R² are each optionally substituted with 1, 2, 3, or 4 independently selected R^{2A} substituents.

In some embodiments, each R^{a21}, R^{c21}, and R^{d21} is independently selected from H, C₁₋₆ alkyl, C₁₋₆ haloalkyl, C₂₋₆ alkenyl, and C₂₋₆ alkynyl.

In some embodiments, each R^{a21}, R^{c21}, and R^{d21} is independently selected from H, C₁₋₆ alkyl, and C₁₋₆ haloalkyl.

In some embodiments, each R^{a21}, R^{c21}, and R^{d21} is independently selected from H, C₁₋₆ alkyl.

In some embodiments, each R^{a21}, R^{c21}, and R^{d21} is independently selected from H, C₁₋₃ alkyl.

In some embodiments, each R^{a21}, R^{c21}, and R^{d21} is independently selected from H and methyl.

In some embodiments, each R² is independently selected from C₁₋₆ alkyl, C₁₋₆ haloalkyl, C₃₋₁₀ cycloalkyl, 5-10 membered heteroaryl, 4-10 membered heterocycloalkyl, OR^{a21}, NR^{c21}R^{d21}, C(O)NR^{c21}R^{d21}, and NR^{c21}C(O)OR^{a21}, wherein the C₁₋₆ alkyl, C₃₋₁₀ cycloalkyl, 5-10 membered heteroaryl, and 4-10 membered heterocycloalkyl of R² are each optionally substituted with 1, 2, 3, or 4 independently selected R^{2A} substituents; and
each R^{a21}, R^{c21}, and R^{d21} is independently selected from H, C₁₋₆ alkyl, C₁₋₆ haloalkyl, C₂₋₆ alkenyl, and C₂₋₆ alkynyl.

In some embodiments, each R² is independently selected from C₁₋₆ alkyl, C₁₋₆ haloalkyl, C₃₋₁₀ cycloalkyl, 5-10 membered heteroaryl, 4-10 membered heterocycloalkyl, OR^{a21}, C(O)NR^{c21}R^{d21}, and NR^{c21}C(O)OR^{a21}, wherein the C₁₋₆ alkyl, C₃₋₁₀ cycloalkyl, 5-10 membered heteroaryl, and 4-10 membered heterocycloalkyl of R² are each optionally substituted with 1, 2, 3, or 4 independently selected R^{2A} substituents; and
each R^{a21}, R^{c21}, and R^{d21} is independently selected from H, C₁₋₆ alkyl, C₁₋₆ haloalkyl, C₂₋₆ alkenyl, and C₂₋₆ alkynyl.

In some embodiments, each R² is independently selected from C₁₋₆ alkyl, C₁₋₆ haloalkyl, C₃₋₇ cycloalkyl, 5-6 membered heteroaryl, 4-7 membered heterocycloalkyl, OR^{a21}, NR^{c21}R^{d21}, C(O)NR^{c21}R^{d21}, and NR^{c21}C(O)OR^{a21}, wherein the C₁₋₆ alkyl, C₃₋₇ cycloalkyl, 5-6 membered heteroaryl, and 4-7 membered heterocycloalkyl of R² are each optionally substituted with 1, 2, 3, or 4 independently selected R^{2A} substituents; and
each R^{a21}, R^{c21}, and R^{d21} is independently selected from H, C₁₋₆ alkyl, and C₁₋₆ haloalkyl.

In some embodiments, each R² is independently selected from C₁₋₆ alkyl, C₁₋₆ haloalkyl, C₃₋₇ cycloalkyl, 5-6 membered heteroaryl, 4-7 membered heterocycloalkyl, OR^{a21}, C(O)NR^{c21}R^{d21}, and NR^{c21}C(O)OR^{a21}, wherein the C₁₋₆ alkyl, C₃₋₇ cycloalkyl, 5-6 membered heteroaryl, and 4-7 membered heterocycloalkyl of R² are each optionally substituted with 1, 2, 3, or 4 independently selected R^{2A} substituents; and
each R^{a21}, R^{c21}, and R^{d21} is independently selected from H, C₁₋₆ alkyl, and C₁₋₆ haloalkyl.

In some embodiments, each R^{2A} is independently selected from OH, halo, oxo, CN, C(O)OH, NH₂, NO₂, SF₅, C₁₋₆ alkyl, C₁₋₆alkoxy, C₁₋₆haloalkoxy, C₁₋₆haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments, each R^{2A} is independently selected from OH, halo, oxo, CN, C(O)OH, NH₂, NO₂, SF₅, C₁₋₆ alkyl, C₁₋₆alkoxy, C₁₋₆haloalkoxy, and C₁₋₆haloalkyl.

In some embodiments, each R^{2A} is independently selected from OH, halo, oxo, CN, C(O)OH, C₁₋₆ alkyl, C₁₋₆alkoxy, C₁₋₆haloalkoxy, and C₁₋₆haloalkyl.

In some embodiments, each R^{2A} is independently selected from OH, halo, oxo, C(O)OH, C₁₋₆ alkyl, C₁₋₆alkoxy, C₁₋₆haloalkoxy, and C₁₋₆haloalkyl.

In some embodiments, each R^{2A} is independently selected from OH, halo, C₁₋₆ alkyl, C₁₋₆ alkoxy, CN, and oxo.

In some embodiments, each R^{2A} is independently selected from OH, halo, C₁₋₆ alkyl, C₁₋₆ alkoxy, and oxo.

In some embodiments, each R^{2A} is independently selected from OH, halo, and oxo.

In some embodiments, each R^{2A} is independently selected from OH, fluoro, methyl, methoxy, ethoxy, and CN.

In some embodiments, each R^{2A} is independently selected from OH, fluoro, methyl, methoxy, and ethoxy.

In some embodiments, each R^{2A} is OH.

In some embodiments, each R² is independently selected from methyl, trideuteromethyl, ethyl, n-propyl, isopropyl, difluoromethyl, trifluoromethyl, trifluoroethyl, methoxy, trideuteromethoxy, ethoxy, trideuteroethoxy, dimethylamino, di(trideuteromethyl)amino, —NHC(O)OCH₃, —N(CH₃)C(O)OCH₃, —N(CH₃)C(O)OCH₂CH₃, —C(O)N(CH₃)₂, cyclopropyl, cyclobutyl, cyclohexyl, pyrazolyl, pyridinyl, azetidinyl, morpholinyl, pyrrolidinonyl, imidazolidinonyl, tetrahydropyranyl, and azabicyclo[2.1.1]hexanyl, wherein the methyl, ethyl, n-propyl, isopropyl, cyclopropyl, cyclobutyl, cyclohexyl, pyrazolyl, pyridinyl, azetidinyl, morpholinyl, pyrrolidinonyl, imidazolidinonyl, and tetrahydropyranyl of R² are each optionally substituted with 1 or 2 independently selected R^{2A} substituents.

In some embodiments, each R² is independently selected from methyl, trideuteromethyl, ethyl, n-propyl, isopropyl, difluoromethyl, trifluoromethyl, trifluoroethyl, methoxy, trideuteromethoxy, ethoxy, trideuteroethoxy, dimethylamino, di(trideuteromethyl)amino, —NHC(O)OCH₃, —N(CH₃)C(O)OCH₃, —N(CH₃)C(O)OCH₂CH₃, —C(O)N(CH₃)₂, cyclopropyl, cyclobutyl, cyclohexyl, pyrazolyl, pyridinyl, azetidinyl, morpholinyl, pyrrolidinonyl, imidazolidinonyl, tetrahydropyranyl, and azabicyclo[2.1.1]hexanyl, wherein the methyl, ethyl, n-propyl, isopropyl, cyclopropyl, cyclobutyl, cyclohexyl, pyrazolyl, pyridinyl, azetidinyl, morpholinyl, pyrrolidinonyl, imidazolidinonyl, and tetrahydropyranyl of R² are each optionally substituted with 1 or 2 independently selected R^{2A} substituents; and
each R^{2A} is independently selected from OH, halo, C₁₋₆ alkyl, C₁₋₆ alkoxy, CN, and oxo.

In some embodiments, each R² is independently selected from methyl, trideuteromethyl, ethyl, n-propyl, isopropyl, difluoromethyl, trifluoromethyl, trifluoroethyl, methoxy, trideuteromethoxy, ethoxy, trideuteroethoxy, dimethylamino, di(trideuteromethyl)amino, —NHC(O)OCH₃, —N(CH₃)C(O)OCH₃, —N(CH₃)C(O)OCH₂CH₃, —C(O)N(CH₃)₂, cyclopropyl, cyclobutyl, cyclohexyl, pyrazolyl, pyridinyl, azetidinyl, morpholinyl, pyrrolidinonyl, imidazolidinonyl, tetrahydropyranyl, and azabicyclo[2.1.1]hexanyl, wherein the methyl, ethyl, n-propyl, isopropyl, cyclopropyl, cyclobutyl, cyclohexyl, pyrazolyl, pyridinyl, azetidinyl, morpholinyl, pyrrolidinonyl, imidazolidinonyl, and tetrahydropyranyl of R² are each optionally substituted with 1 or 2 independently R^{2A} substituents independently selected from OH, fluoro, methyl, methoxy, ethoxy, and CN.

In some embodiments, each R² is independently selected from methyl, trideuteromethyl, ethyl, n-propyl, isopropyl, difluoromethyl, trifluoromethyl, trifluoroethyl, methoxy, ethoxy, —NHC(O)OCH₃, —C(O)N(CH₃)₂, cyclopropyl, cyclobutyl, cyclohexyl, pyrazolyl, pyridinyl, azetidinyl, morpholinyl, pyrrolidinonyl, imidazolidinonyl, and tetrahydropyranyl, wherein the methyl, ethyl, n-propyl, isopropyl, cyclopropyl, cyclobutyl, cyclohexyl, pyrazolyl, pyridinyl, azetidinyl, morpholinyl, pyrrolidinonyl, imidazolidinonyl, and tetrahydropyranyl of R² are each optionally substituted with 1 or 2 independently selected R^{2A} substituents.

In some embodiments, each R² is independently selected from methyl, ethyl, n-propyl, isopropyl, difluoromethyl, trifluoromethyl, cyclopropyl, cyclobutyl, cyclohexyl, and tetrahydropyranyl, wherein the methyl, ethyl, n-propyl, isopropyl, cyclopropyl, cyclobutyl, cyclohexyl, and tetrahydropyranyl of R² are each optionally substituted with 1 or 2 independently selected R^{2A} substituents.

In some embodiments, each R² is independently selected from methyl, trideuteromethyl, ethyl, n-propyl, isopropyl, difluoromethyl, trifluoromethyl, trifluoroethyl, methoxy, ethoxy, —NHC(O)OCH₃, —C(O)N(CH₃)₂, cyclopropyl, cyclobutyl, cyclohexyl, pyrazolyl, pyridinyl, azetidinyl, morpholinyl, pyrrolidinonyl, imidazolidinonyl, and tetrahydropyranyl, wherein the methyl, ethyl, n-propyl, isopropyl, cyclopropyl, cyclobutyl, cyclohexyl, pyrazolyl, pyridinyl, azetidinyl, morpholinyl, pyrrolidinonyl, imidazolidinonyl, and tetrahydropyranyl of R² are each optionally substituted with 1 or 2 R^{2A} substituents; and
each R^{2A} is independently selected from OH, halo, C₁₋₆ alkyl, C₁₋₆ alkoxy, and oxo.

In some embodiments, each R² is independently selected from methyl, ethyl, n-propyl, isopropyl, difluoromethyl, trifluoromethyl, cyclopropyl, cyclobutyl, cyclohexyl, and tetrahydropyranyl, wherein the methyl, ethyl, n-propyl, isopropyl, cyclopropyl, cyclobutyl, cyclohexyl, and tetrahydropyranyl of R² are each optionally substituted with 1 or 2 independently selected R^{2A} substituents; and
each R^{2A} is independently selected from OH, halo, and oxo.

In some embodiments, each R² is independently selected from methyl, trideuteromethyl, ethyl, n-propyl, isopropyl, difluoromethyl, trifluoromethyl, trifluoroethyl, methoxy, ethoxy, —NHC(O)OCH₃, —C(O)N(CH₃)₂, cyclopropyl, cyclobutyl, cyclohexyl, pyrazolyl, pyridinyl, azetidinyl, morpholinyl, pyrrolidinonyl, imidazolidinonyl, and tetrahydropyranyl, wherein the methyl, ethyl, n-propyl, isopropyl, cyclopropyl, cyclobutyl, cyclohexyl, pyrazolyl, pyridinyl, azetidinyl, morpholinyl, pyrrolidinonyl, imidazolidinonyl, and tetrahydropyranyl of R² are each optionally substituted with 1 or 2 R^{2A} substituents independently selected from fluoro, methyl, methoxy, ethoxy, and hydroxy.

In some embodiments, each R² is independently selected from methyl, ethyl, n-propyl, isopropyl, difluoromethyl, trifluoromethyl, cyclopropyl, cyclobutyl, cyclohexyl, and tetrahydropyranyl, wherein the methyl, ethyl, n-propyl, isopropyl, cyclopropyl, cyclobutyl, cyclohexyl, and tetrahydropyranyl of R² are each optionally substituted with 1 or 2 hydroxy groups.

In some embodiments, each R² is independently selected from methyl, trideuteromethyl, methoxymethyl, trideuteromethoxymethyl, ethyl, hydroxyethyl, methoxyethyl, trideuteromethoxyethyl, hydroxypropyl, isopropyl, hydroxyisopropyl, difluoromethyl, trifluoromethyl, trifluoroethyl, methoxy, trideuteromethoxy, ethoxy, trideuteroethoxy, dimethylamino, di(trideuteromethyl)amino, —NHC(O)OCH₃, —N(CH₃)C(O)OCH₃, —N(CH₃)C(O)OCH₂CH₃, —C(O)N(CH₃)₂, difluoroazetidinyl, morpholinyl, pyrrolidinonyl, methylimidazolidinonyl, methylpyrazolyl, methoxypyridinyl, cyclopropyl, cyanocyclopropyl, cyclobutyl, hydroxycyclohexyl, tetrahydropyranyl, and (fluoro)azabicyclo[2.1.1]hexanyl.

In some embodiments, each R² is independently selected from methyl, trideuteromethyl, ethyl, hydroxyethyl, hydroxypropyl, isopropyl, hydroxyisopropyl, difluoromethyl, trifluoromethyl, trifluoroethyl, methoxy, ethoxy, —NHC(O)OCH₃, —C(O)N(CH₃)₂, difluoroazetidinyl, morpholinyl, pyrrolidinonyl, methylimidazolidinonyl, methylpyrazolyl, methoxypyridinyl, cyclopropyl, cyclobutyl, hydroxycyclohexyl, and tetrahydropyranyl.

In some embodiments, each R² is independently selected from methyl, hydroxyethyl, hydroxypropyl, hydroxyisopropyl, difluoromethyl, trifluoromethyl, cyclopropyl, cyclobutyl, hydroxycyclohexyl, and tetrahydropyranyl.

In some embodiments, p is 0 or 1.

In some embodiments, p is 0.

In some embodiments, p is 1.

In some embodiments, each R³ is independently selected from halo and C₁₋₃ alkoxy.

In some embodiments, each R³ is independently selected from C₁₋₃ alkoxy.

In some embodiments, each R³ is independently selected from halo.

In some embodiments, each R³ is methoxy.

In some embodiments, each R³ is fluoro.

In some embodiments, R⁵ is methyl.

In some embodiments, R⁵ is trideuteromethyl.

In some embodiments of Formula I or Formula Ia:
Cy¹ is selected from

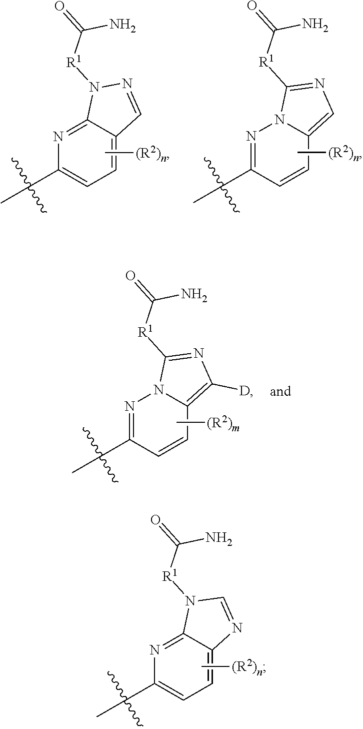

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, C₁₋₃ alkyl, C₁₋₃ haloalkyl, C₁₋₃ alkoxy, C₂₋₃ alkynyl, and C₃₋₅ cycloalkyl;
each R² is independently selected from C₁₋₆ alkyl, C₁₋₆ haloalkyl, C₃₋₇ cycloalkyl, 5-6 membered heteroaryl, 4-7 membered heterocycloalkyl, OR^{a21}, NR^{c21}R^{d21}, C(O)NR^{c21}R^{d21}, and NR^{c21}C(O)OR^{a21}, wherein each C₁₋₆ alkyl, C₃₋₇ cycloalkyl, 5-6 membered heteroaryl, and 4-7 membered heterocycloalkyl is optionally substituted with 1 or 2 R^{2A} substituents independently selected from fluoro, methyl, methoxy, ethoxy, CN, and hydroxy;
each R^{a21}, R^{c21}, and R^{d21} is independently selected from H and C₁₋₃ alkyl;
each R³ is independently selected from halo and C₁₋₃ alkoxy;
R⁴ is H or D;
R⁵ is methyl or trideuteromethyl;
m is 0, 1, or 2;
n is 0, 1, 2, or 3; and
p is 0, 1, or 2.

In some embodiments of Formula I or Formula Ia:
Cy¹ is selected from and

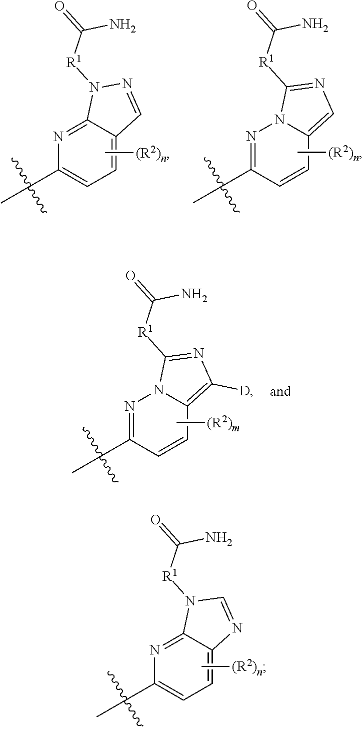

R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, C₁₋₃ alkyl, C₁₋₃ haloalkyl, C₁₋₃ alkoxy, C₂₋₄ alkynyl, and C₃₋₅ cycloalkyl;
each R² is independently selected from C₁₋₆ alkyl, C₁₋₆ haloalkyl, C₃₋₁₀ cycloalkyl, 5-10 membered heteroaryl, 4-10 membered heterocycloalkyl, OR^{a21}, NR^{c21}R^{d21}, C(O)NR^{c21}R^{d21} and NR^{c21}C(O)OR^{a21}, wherein the C₁₋₆ alkyl, C₃₋₁₀ cycloalkyl, 5-10 membered heteroaryl, and 4-10 membered heterocycloalkyl of R² are each optionally substituted with 1, 2, 3, or 4 independently selected R^{2A} substituents; and
each R^{a21}, R^{c21}, and R^{d21} is independently selected from H, C₁₋₆ alkyl, C₁₋₆ haloalkyl, C₂₋₆ alkenyl, and C₂₋₆ alkynyl;
each R³ is independently selected from halo and C₁₋₃ alkoxy;
R⁴ is H or D;
R⁵ is methyl or trideuteromethyl;
m is 0, 1, or 2;
n is 0, 1, 2, or 3;
p is 0, 1, or 2.

In some embodiments of Formula I or Formula Ia:
Cy¹ is selected from

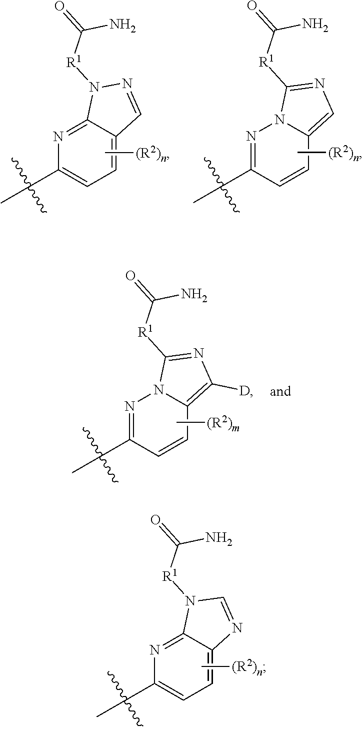

R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, C₁₋₃ alkyl, C₁₋₃ haloalkyl, C₁₋₃ alkoxy, C₂₋₄ alkynyl, and C₃₋₅ cycloalkyl;
each R² is independently selected from methyl, trideuteromethyl, ethyl, n-propyl, isopropyl, difluoromethyl, trifluoromethyl, trifluoroethyl, methoxy, trideuteromethoxy, ethoxy, trideuteroethoxy, dimethylamino, di(trideuteromethyl)amino, —NHC(O)OCH₃, —N(CH₃)C(O)OCH₃, —N(CH₃)C(O)OCH₂CH₃, —C(O)N(CH₃)₂, cyclopropyl, cyclobutyl, cyclohexyl, pyrazolyl, pyridinyl, azetidinyl, morpholinyl, pyrrolidinonyl, imidazolidinonyl, tetrahydropyranyl, and azabicyclo[2.1.1]hexanyl, wherein the methyl, ethyl, n-propyl, isopropyl, cyclopropyl, cyclobutyl, cyclohexyl, pyrazolyl, pyridinyl, azetidinyl, morpholinyl, pyrrolidinonyl, imidazolidinonyl, and tetrahydropyranyl of R² are each optionally substituted with 1 or 2 R^{2A} substituents independently selected from fluoro, methyl, methoxy, trideuteromethoxy, ethoxy, cyano, and hydroxy;
each R³ is independently selected from fluoro and methoxy;
R⁴ is H or D;
R⁵ is methyl or trideuteromethyl;
m is 0;
n is 0, 1, or 2;
p is 0 or 1.

In some embodiments of Formula I or Formula Ia:
Cy¹ is selected from

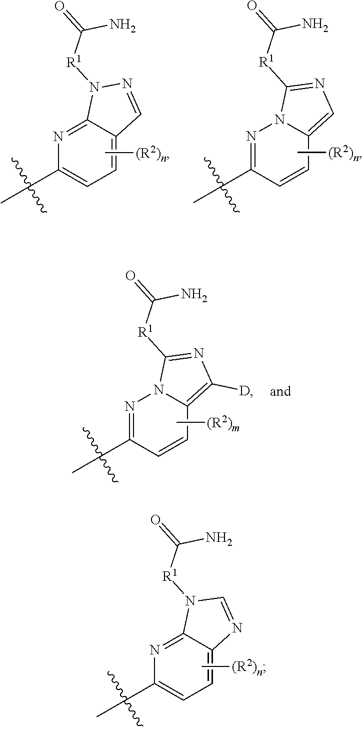

R¹ is selected from

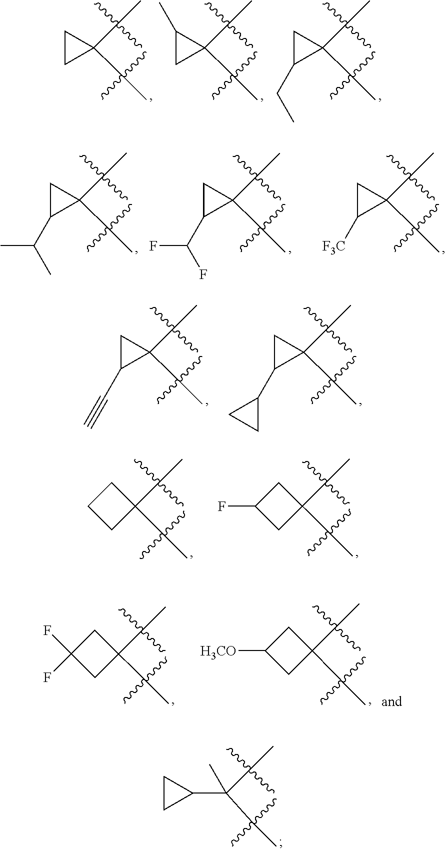

each R² is independently selected from methyl, trideuteromethyl, methoxymethyl, trideuteromethoxymethyl, ethyl, hydroxyethyl, methoxyethyl, trideuteromethoxyethyl, hydroxypropyl, isopropyl, hydroxyisopropyl, difluoromethyl, trifluoromethyl, trifluoroethyl, methoxy, trideuteromethoxy, ethoxy, trideuteroethoxy, dimethylamino, di(trideuteromethyl)amino, —NHC(O)OCH₃, —N(CH₃)C(O)OCH₃, —N(CH₃)C(O)OCH₂CH₃, —C(O)N(CH₃)₂, difluoroazetidinyl, morpholinyl, pyrrolidinonyl, methylimidazolidinonyl, methylpyrazolyl, methoxypyridinyl, cyclopropyl, cyanocyclopropyl, cyclobutyl, hydroxycyclohexyl, tetrahydropyranyl, and (fluoro)azabicyclo[2.1.1]hexanyl;
each R³ is independently selected from fluoro and methoxy;
R⁴ is H or D;
R⁵ is methyl or trideuteromethyl;
m is 0;
n is 0, 1, or 2; and
p is 0 or 1.

In some embodiments of Formula I or Formula Ia:
Cy¹ is selected from

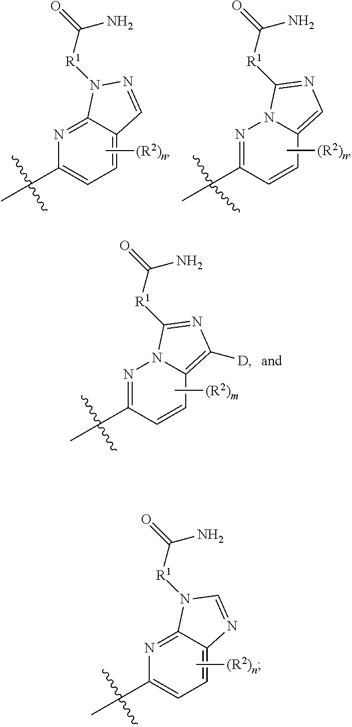

R¹ is selected from

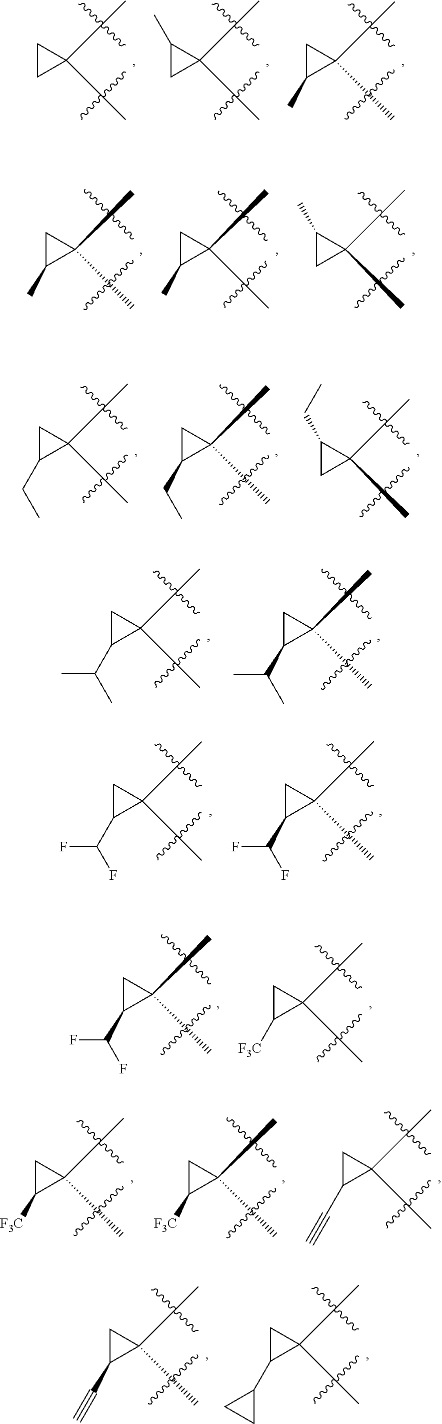

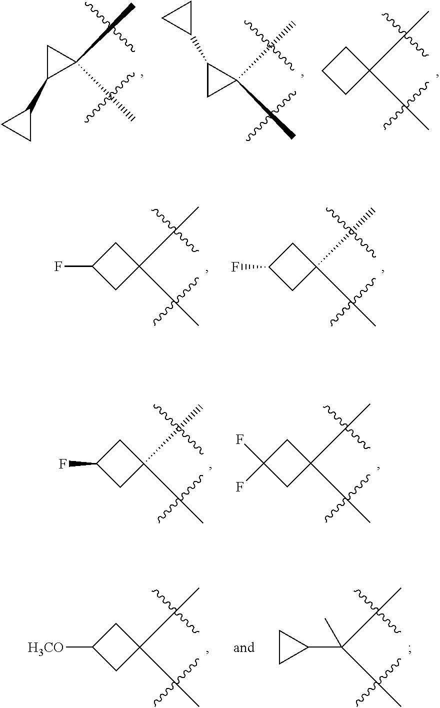

each R² is independently selected from methyl, trideuteromethyl, methoxymethyl, trideuteromethoxymethyl, ethyl, hydroxyethyl, methoxyethyl, trideuteromethoxyethyl, hydroxypropyl, isopropyl, hydroxyisopropyl, difluoromethyl, trifluoromethyl, trifluoroethyl, methoxy, trideuteromethoxy, ethoxy, trideuteroethoxy, dimethylamino, di(trideuteromethyl)amino, —NHC(O)OCH₃, —N(CH₃)C(O)OCH₃, —N(CH₃)C(O)OCH₂CH₃, —C(O)N(CH₃)₂, difluoroazetidinyl, morpholinyl, pyrrolidinonyl, methylimidazolidinonyl, methylpyrazolyl, methoxypyridinyl, cyclopropyl, cyanocyclopropyl, cyclobutyl, hydroxycyclohexyl, tetrahydropyranyl, and (fluoro)azabicyclo[2.1.1]hexanyl;
each R³ is independently selected from fluoro and methoxy;
R⁴ is H or D;
R⁵ is methyl or trideuteromethyl;
m is 0;
n is 0, 1, or 2; and
p is 0 or 1.

In some embodiments of Formula I or Formula Ia:
Cy¹ is selected from

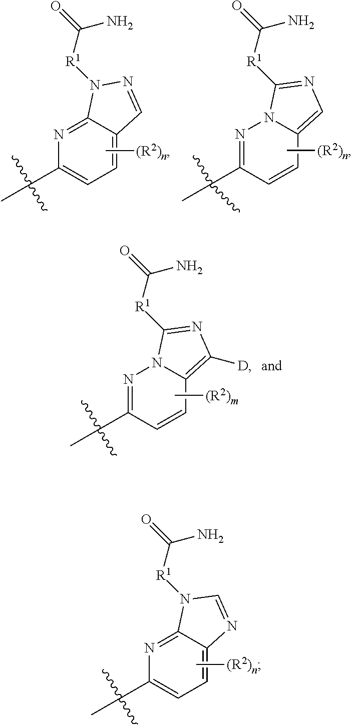

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, C₁₋₃ alkyl, C₁₋₃ alkoxy, C₂₋₃ alkynyl, and C₃₋₅ cycloalkyl;
each R² is independently selected from C₁₋₆ alkyl, C₁₋₆ haloalkyl, C₃₋₇ cycloalkyl, 5-6 membered heteroaryl, 4-7 membered heterocycloalkyl, OR^{a21}, C(O)NR^{c21}R^{d21}, and NR^{c21}C(O)OR^{a21}, wherein each C₁₋₆ alkyl, C₃₋₇ cycloalkyl, 5-6 membered heteroaryl, and 4-7 membered heterocycloalkyl is optionally substituted with 1 or 2 R^{2A} substituents independently selected from fluoro, methyl, methoxy, ethoxy, and hydroxy;
each R^{a21}, R^{c21}, and R^{d21} is independently selected from H and C₁₋₃ alkyl;
each R³ is independently selected from halo;
R⁴ is H or D;
m is 0, 1, or 2;
n is 0, 1, 2, or 3; and
p is 0, 1, or 2.

In some embodiments of Formula I:
Cy¹ is selected from

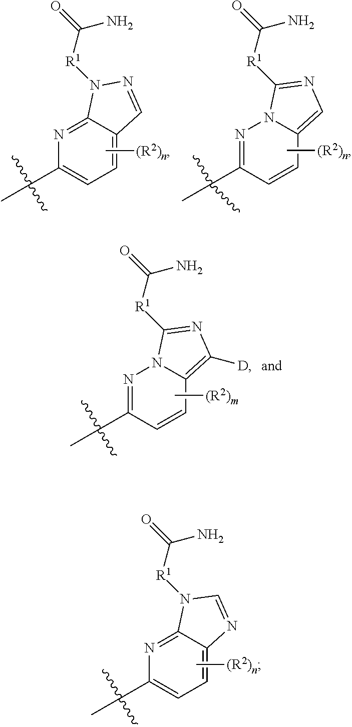

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, C₁₋₃ alkyl, and C₁₋₃ alkoxy;
each R² is independently selected from C₁₋₆ alkyl, C₁₋₆ haloalkyl, C₃₋₇ cycloalkyl, and 4-7 membered heterocycloalkyl, wherein each C₁₋₆ alkyl, C₃₋₇ cycloalkyl, and 4-7 membered heterocycloalkyl is optionally substituted with 1 or 2 independently selected R^{2A} substituents;
each R^{2A} is independently selected from OH, halo, oxo, CN, C(O)OH, NH₂, NO₂, SF₅, C₁₋₆ alkyl, C₁₋₆alkoxy, C₁₋₆haloalkoxy, C₁₋₆haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
m is 0, 1, or 2;
n is 0, 1, 2, or 3; and
p is 0, 1, or 2.

In some embodiments of Formula I or Formula Ia:
Cy¹ is selected from

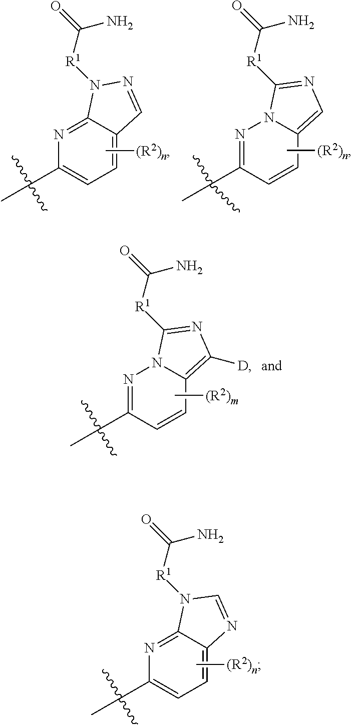

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, C₁₋₃ alkyl, C₁₋₃ alkoxy, C₂₋₄ alkynyl, and C₃₋₅ cycloalkyl;
each R² is independently selected from C₁₋₆ alkyl, C₁₋₆ haloalkyl, C₃₋₇ cycloalkyl, 5-6 membered heteroaryl, 4-7 membered heterocycloalkyl, OR^{a21}, C(O)NR^{c21}R^{d21}, and NR^{c21}C(O)OR^{a21}, wherein the C₁₋₆ alkyl, C₃₋₇ cycloalkyl, and 5-6 membered heteroaryl, 4-7 membered heterocycloalkyl of R² are each optionally substituted with 1, 2, 3, or 4 independently selected R^{2A} substituents;
each R^{a21}, R^{c21}, and R^{d21} is independently selected from H, C₁₋₆ alkyl, and C₁₋₆ haloalkyl;
each R^{2A} is independently selected from OH, halo, oxo, C(O)OH, C₁₋₆ alkyl, C₁₋₆ alkoxy, C₁₋₆ haloalkoxy, and C₁₋₆ haloalkyl;
each R³ is independently selected from halo;
R⁴ is H or D;
m is 0, 1, or 2;
n is 0, 1, 2, or 3; and
p is 0 or 1, or 2.

In some embodiments of Formula I:
Cy¹ is selected from

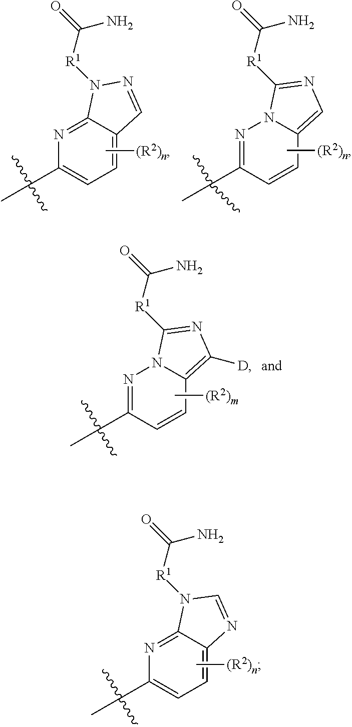

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, C₁₋₃ alkyl, and C₁₋₃ alkoxy;
each R² is independently selected from C₁₋₆ alkyl, C₁₋₆ haloalkyl, C₃₋₇ cycloalkyl, and 4-7 membered heterocycloalkyl, wherein each C₁₋₆ alkyl, C₃₋₇ cycloalkyl, and 4-7 membered heterocycloalkyl is optionally substituted with 1 or 2 independently selected R^{2A} substituents;
each R^{2A} is independently selected from OH, halo, oxo, C(O)OH, C₁₋₆ alkyl, C₁₋₆ alkoxy, C₁₋₆ haloalkoxy, and C₁₋₆ haloalkyl;
m is 0, 1, or 2;
n is 0, 1, 2, or 3; and
p is 0 or 1, or 2.

In some embodiments of Formula I or Formula Ia:
Cy¹ is selected from

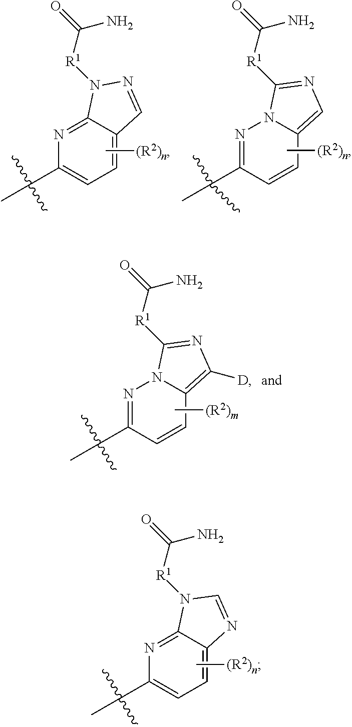

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, C₁₋₃ alkyl, C₁₋₃ alkoxy, C₂₋₄ alkynyl, and C₃₋₅ cycloalkyl;
each R² is independently selected from C₁₋₆ alkyl, C₁₋₆ haloalkyl, C₃₋₇ cycloalkyl, 5-6 membered heteroaryl, 4-7 membered heterocycloalkyl, OR^{a21}, C(O)NR^{c21}R^{d21}, and NR^{c21}C(O)OR^{a21}, wherein the C₁₋₆ alkyl, C₃₋₇ cycloalkyl, 5-6 membered heteroaryl, and 4-7 membered heterocycloalkyl of R² are each optionally substituted with 1, 2, 3, or 4 independently selected R^{2A} substituents independently selected from OH, halo, C₁₋₆ alkyl, C₁₋₆ alkoxy, and oxo;
each R³ is independently selected from halo;
R⁴ is H or D;
m is 0, 1, or 2;
n is 0, 1, 2, or 3; and
p is 0, 1, or 2.

In some embodiments of Formula I:
Cy¹ is selected from

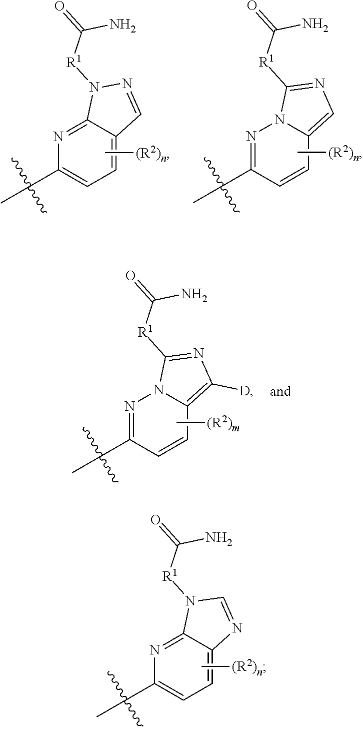

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, C₁₋₃ alkyl, and C₁₋₃ alkoxy;
each R² is independently selected from C₁₋₆ alkyl, C₁₋₆ haloalkyl, C₃₋₇ cycloalkyl, and 4-7 membered heterocycloalkyl, wherein each C₁₋₆ alkyl, C₃₋₇ cycloalkyl, and 4-7 membered heterocycloalkyl is optionally substituted with 1 or 2 hydroxy groups;
m is 0, 1, or 2;
n is 0, 1, 2, or 3; and
p is 0, 1, or 2.

In some embodiments of Formula I or Formula Ia:
Cy¹ is selected from

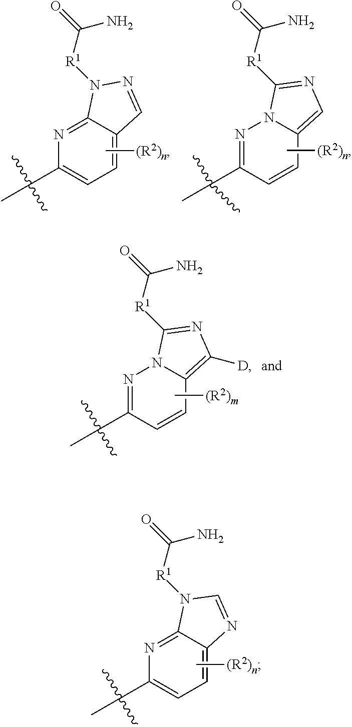

R¹ is selected from R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, C₁₋₃ alkyl, C₁₋₃ alkoxy, C₂₋₄ alkynyl, and C₃₋₅ cycloalkyl;
each R² is independently selected from methyl, ethyl, n-propyl, isopropyl, difluoromethyl, trifluoromethyl, cyclopropyl, cyclobutyl, cyclohexyl, and tetrahydropyranyl, wherein the methyl, ethyl, n-propyl, isopropyl, cyclopropyl, cyclobutyl, cyclohexyl, and tetrahydropyranyl of R² are each optionally substituted with 1 or 2 R^{2A} substituents independently selected from fluoro, methyl, methoxy, ethoxy, and hydroxy;
each R³ is independently selected from halo;
R⁴ is H or D;
m is 0;
n is 0, 1, or 2; and
p is 0 or 1.

In some embodiments of Formula I:
Cy¹ is selected from

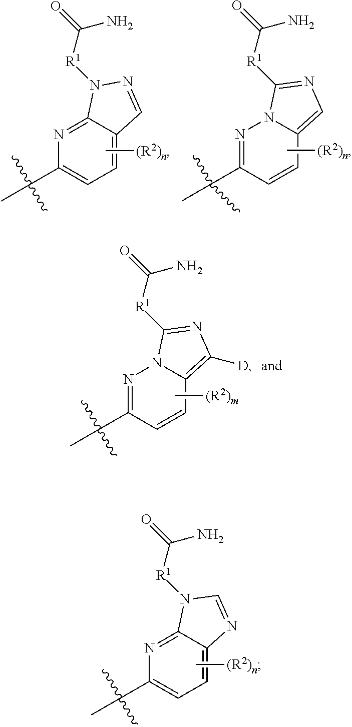

R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, C₁₋₃ alkyl, and C₁₋₃ alkoxy;
each R² is independently selected from methyl, ethyl, n-propyl, isopropyl, difluoromethyl, trifluoromethyl, cyclopropyl, cyclobutyl, cyclohexyl, and tetrahydropyranyl, wherein the methyl, ethyl, n-propyl, isopropyl, cyclopropyl, cyclobutyl, cyclohexyl, and tetrahydropyranyl of R² are each optionally substituted with 1 or 2 hydroxy groups;
m is 0;
n is 0, 1, or 2; and
p is 0.

In some embodiments of Formula I or Formula Ia:
Cy¹ is selected from

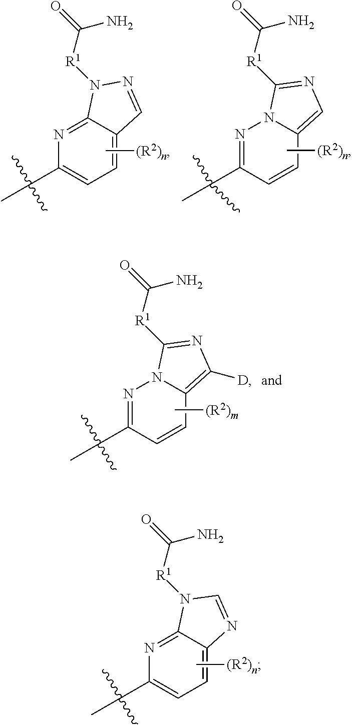

R¹ is selected from

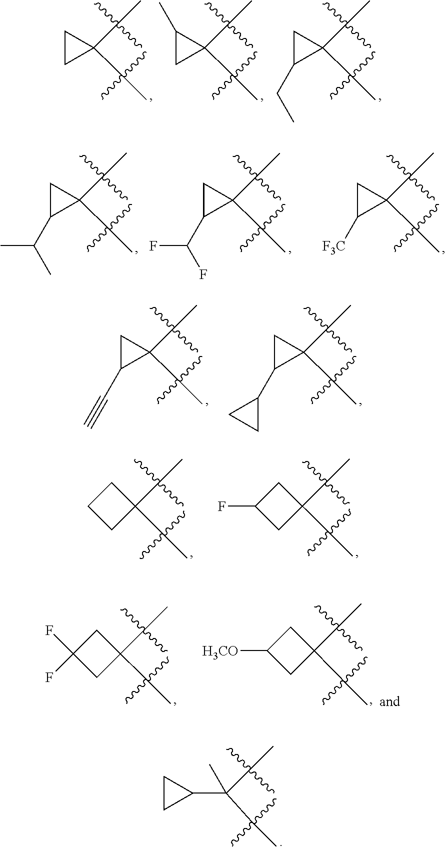

each R² is independently selected from methyl, trideuteromethyl, ethyl, hydroxyethyl, hydroxypropyl, isopropyl, hydroxyisopropyl, difluoromethyl, trifluoromethyl, trifluoroethyl, methoxy, ethoxy, —NHC(O)OCH₃, —C(O)N(CH₃)₂, difluoroazetidinyl, morpholinyl, pyrrolidinonyl, methylimidazolidinonyl, methylpyrazolyl, methoxypyridinyl, cyclopropyl, cyclobutyl, hydroxycyclohexyl, and tetrahydropyranyl;
each R³ is fluoro;
m is 0;
n is 0, 1, or 2; and
p is 0 or 1.

In some embodiments of Formula I:
Cy¹ is selected from

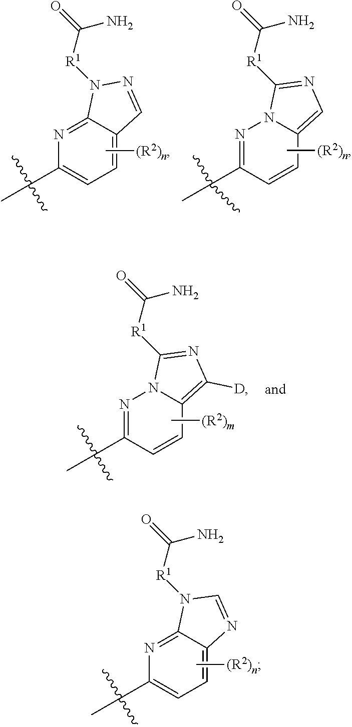

R¹ is selected from

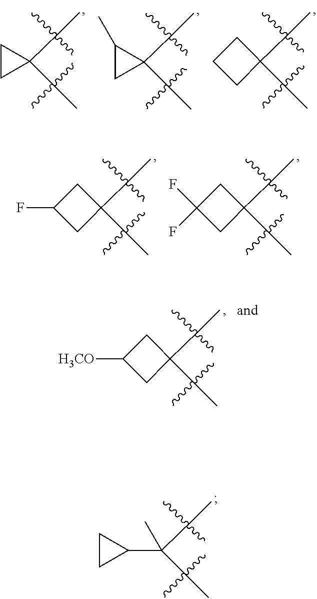

each R² is independently selected from methyl, hydroxyethyl, hydroxypropyl, hydroxyisopropyl, difluoromethyl, trifluoromethyl, cyclopropyl, cyclobutyl, hydroxycyclohexyl, and tetrahydropyranyl;
m is 0;
n is 0, 1, or 2; and
p is 0.

In some embodiments of Formula I or Formula Ia:
Cy¹ is selected from

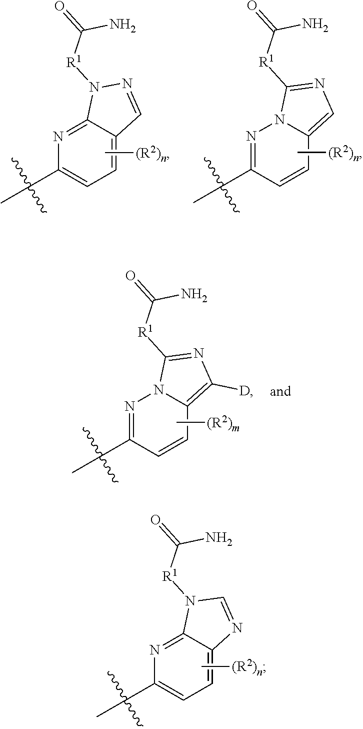

R¹ is selected from

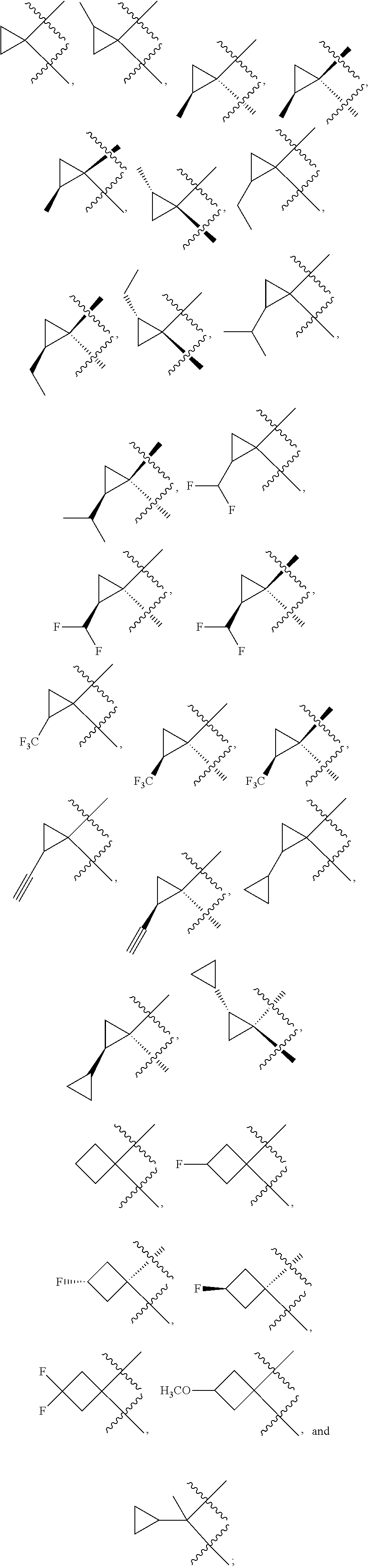

each R² is independently selected from methyl, trideuteromethyl, ethyl, hydroxyethyl, hydroxypropyl, isopropyl, hydroxyisopropyl, difluoromethyl, trifluoromethyl, trifluoroethyl, methoxy, ethoxy, —NHC(O)OCH₃, —C(O)N(CH₃)₂, difluoroazetidinyl, morpholinyl, pyrrolidinonyl, methylimidazolidinonyl, methylpyrazolyl, methoxypyridinyl, cyclopropyl, cyclobutyl, hydroxycyclohexyl, and tetrahydropyranyl;
each R³ is fluoro;
R⁴ is H or D;
m is 0;
n is 0, 1, or 2; and
p is 0 or 1.

In some embodiments of Formula I:
Cy¹ is selected from

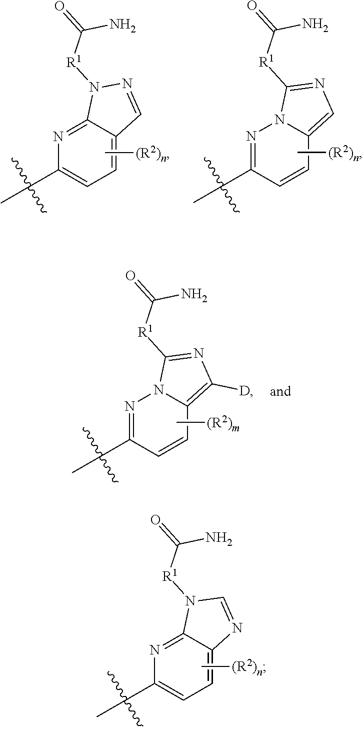

R¹ is selected from

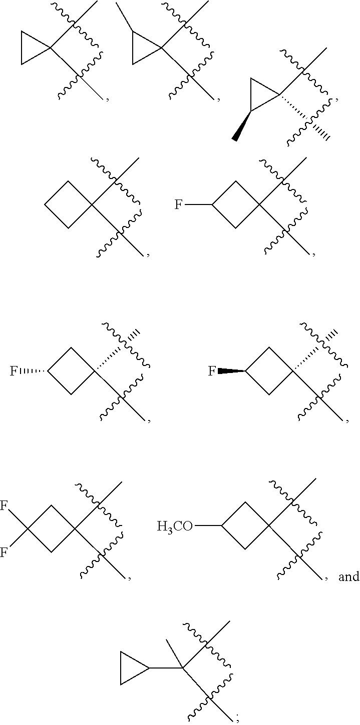

each R² is independently selected from methyl, hydroxyethyl, hydroxypropyl, hydroxyisopropyl, difluoromethyl, trifluoromethyl, cyclopropyl, cyclobutyl, hydroxycyclohexyl, and tetrahydropyranyl;
m is 0;
n is 0, 1, or 2; and
p is 0.

In some embodiments, the compound of Formula I or Formula Ia is a compound of Formula II:

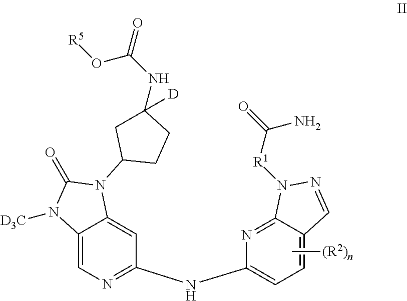

or a pharmaceutically acceptable salt thereof. In some embodiments of Formula II, R⁵ is methyl. In some embodiments of Formula II, R⁵ is trideuteromethyl.

In some embodiments, the compound of Formula I or Formula Ia is a compound of Formula IIa, IIb, IIc, IId, IIe, IIf, IIg, IIh, IIi, IIj, IIk, IIl, IIm, IIn, IIo, IIp, IIq, IIr, IIs, IIt, IIu, IIv, IIw, IIx, IIy, IIz, IIaa, IIab, IIac, IIad, IIae, IIaf, IIag, IIah, IIai, IIaj, IIak, IIal, IIam, IIan, IIao, IIap, IIaq, or IIar:

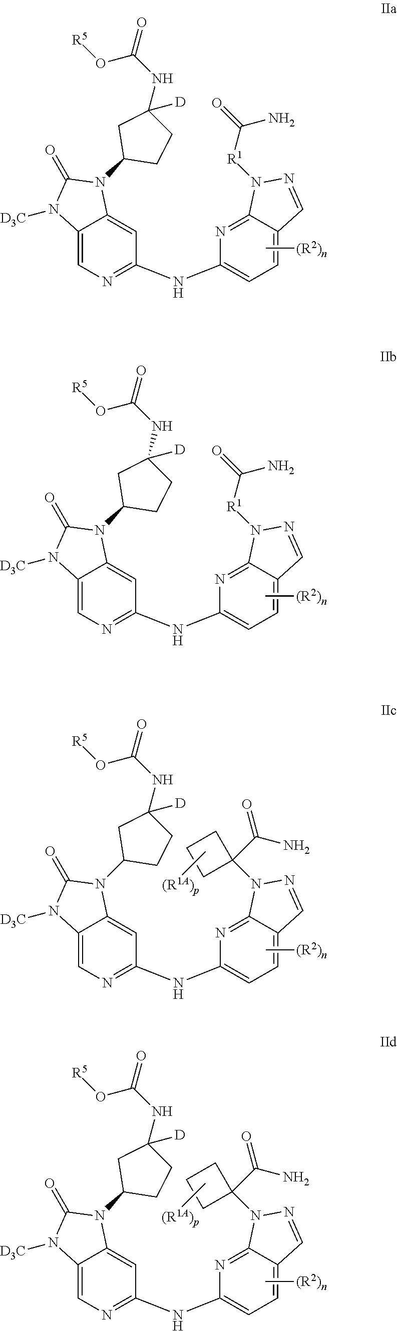

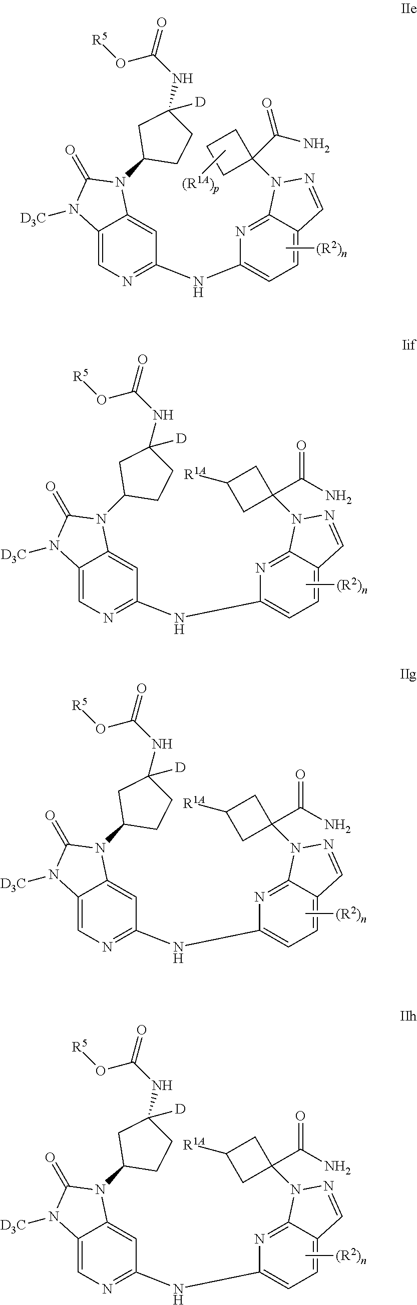

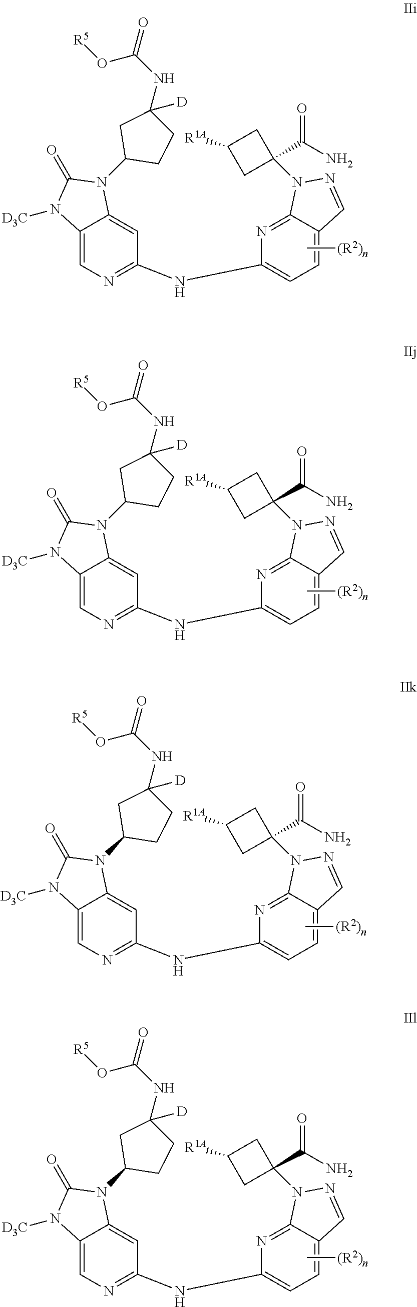

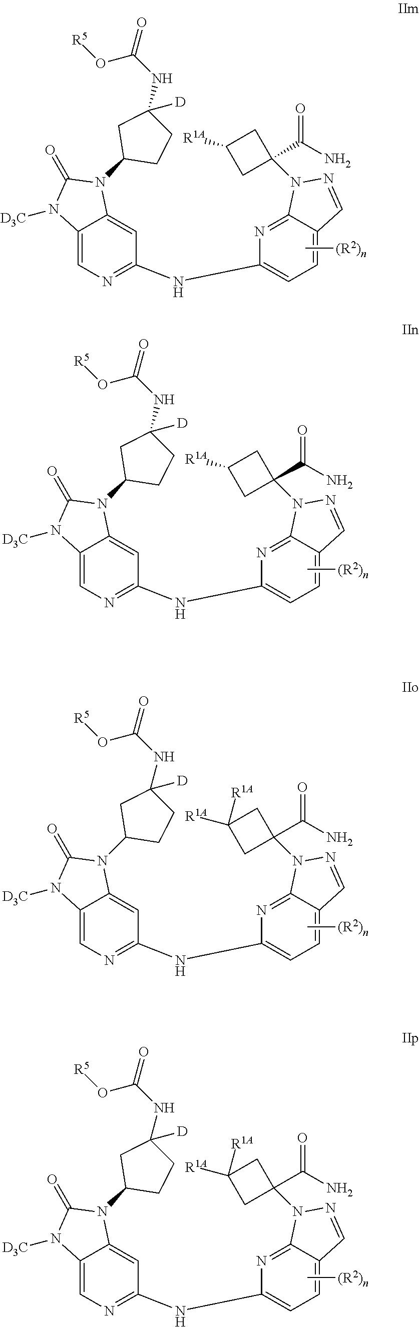

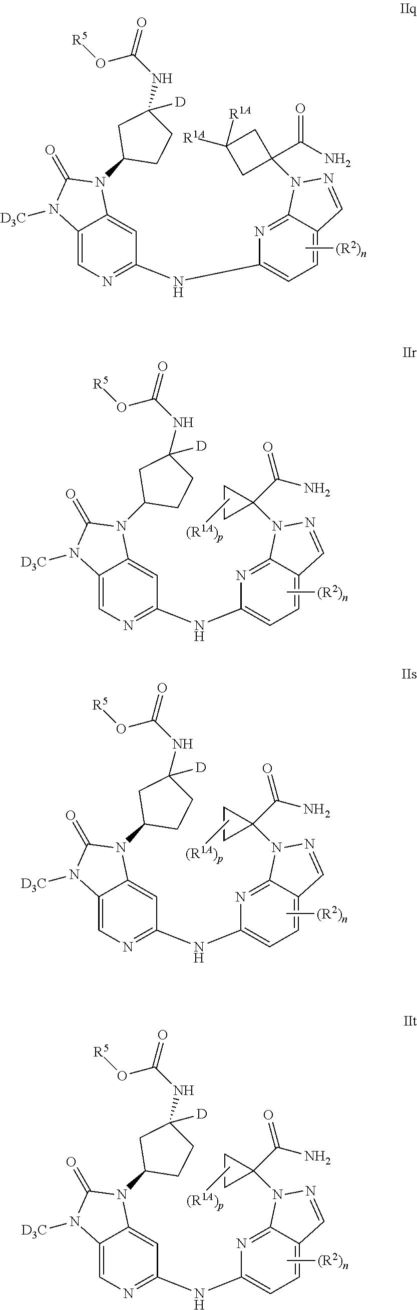

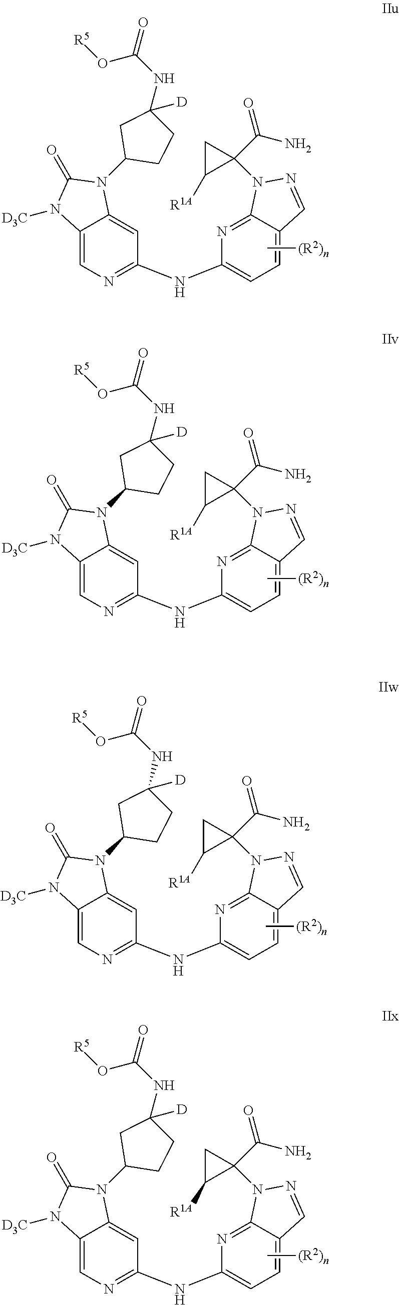

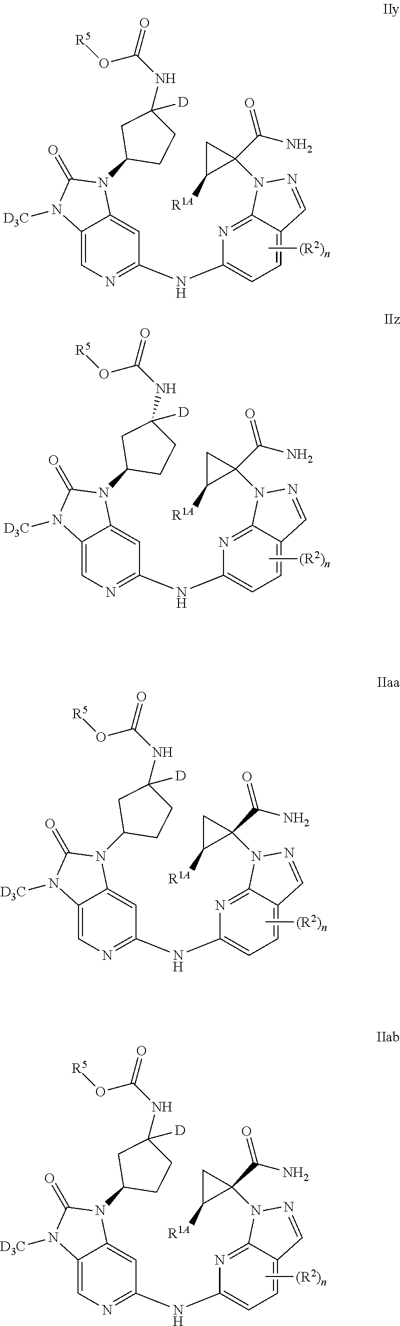

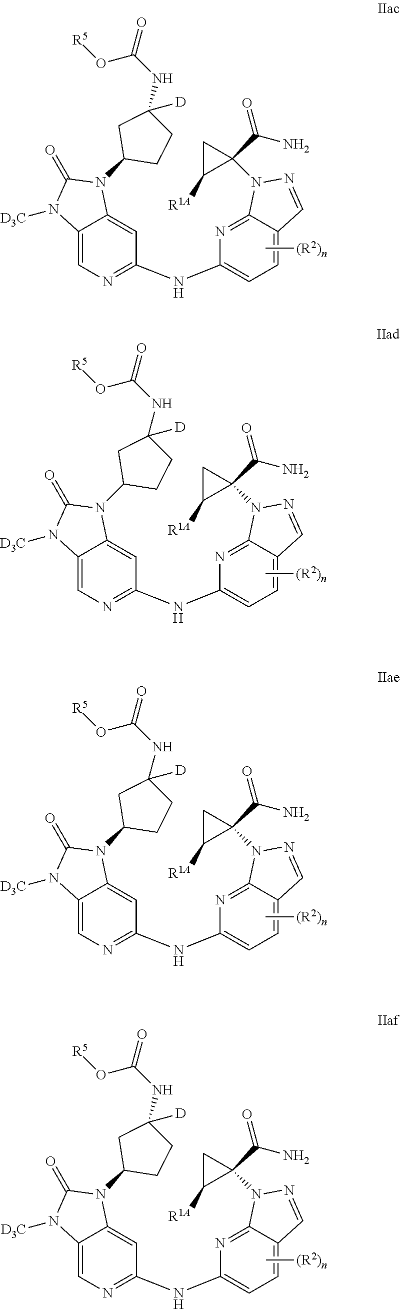

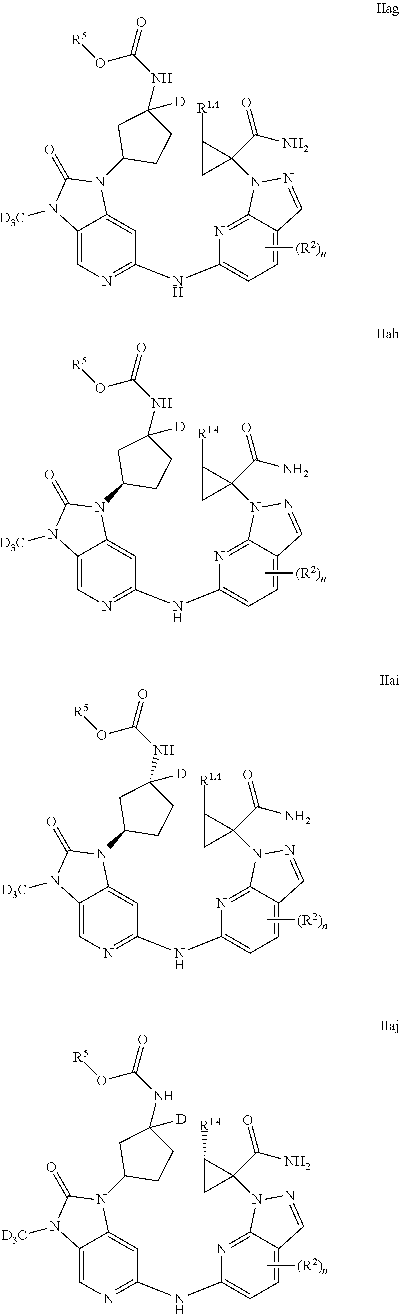

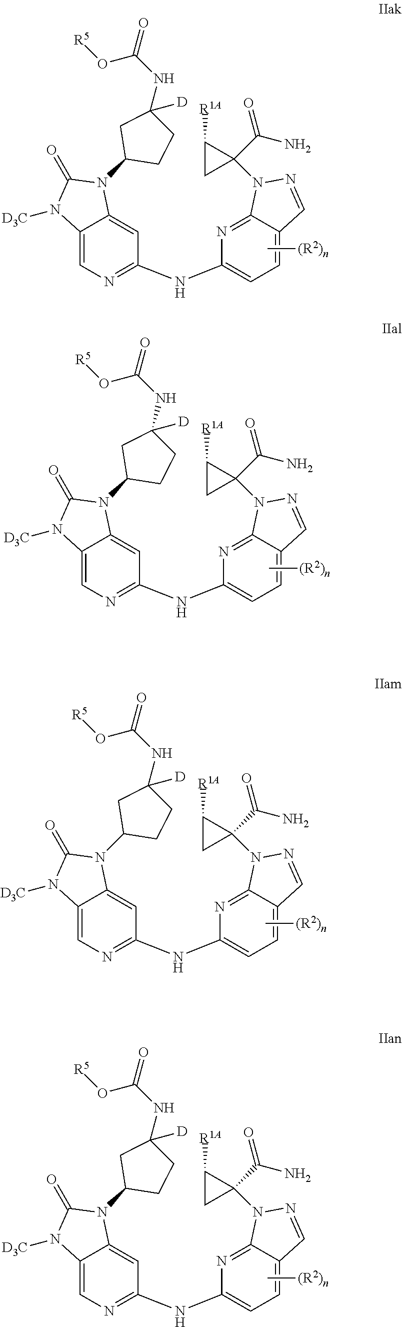

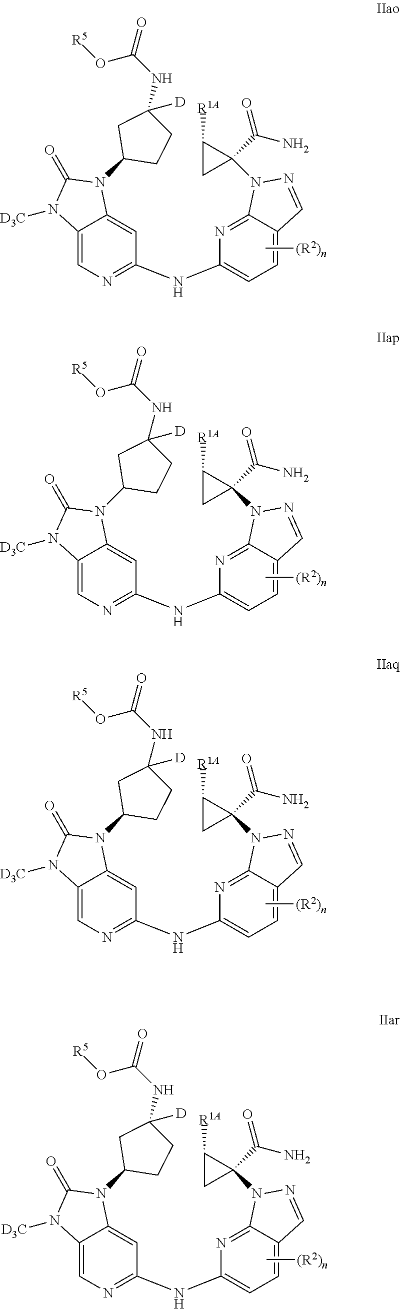

or a pharmaceutically acceptable salt thereof.

In some embodiments, the compound of Formula I or Formula Ia is a compound of Formula IIa, IIb, IIc, IId, IIe, IIf, IIg, IIh, IIi, IIj, IIk, IIl, IIm, IIn, IIo, IIp, IIq, IIr, IIs, IIt, IIu, IIv, IIw, IIx, IIy, IIz, IIaa, IIab, IIac, IIad, IIae, IIaf, IIag, IIah, IIai, IIaj, IIak, IIal, IIam, IIan, IIao, IIap, IIaq, or IIar, wherein R⁵ is methyl.

In some embodiments, the compound of Formula I or Formula Ia is a compound of Formula IIa, IIb, IIc, IId, IIe, IIf, IIg, IIh, IIi, IIj, IIk, IIl, IIm, IIn, IIo, IIp, IIq, IIr, IIs, IIt, IIu, IIv, IIw, IIx, IIy, IIz, IIaa, IIab, IIac, IIad, IIae, IIaf, IIag, IIah, IIai, IIaj, IIak, IIal, IIam, IIan, IIao, IIap, IIaq, or IIar, wherein R⁵ is trideuteromethyl.

In some embodiments, the compound of Formula I or Formula Ia is a compound of Formula III:

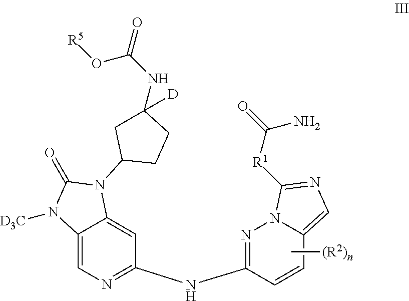

or a pharmaceutically acceptable salt thereof. In some embodiments of Formula III, R⁵ is methyl. In some embodiments of Formula III, R⁵ is trideuteromethyl.

In some embodiments, the compound of Formula I or Formula Ia is a compound of Formula IIIa, IIIb, IIIc, IIId, IIIe, IIIf, IIIg, IIIh, Iiii, IIIj, IIIk, IIIl, IIIm, IIIn, IIIo, IIIp, IIIq, IIIr, IIIs, IIIt, IIIu, IIIv, IIIw, IIIx, IIIy, or IIIz:

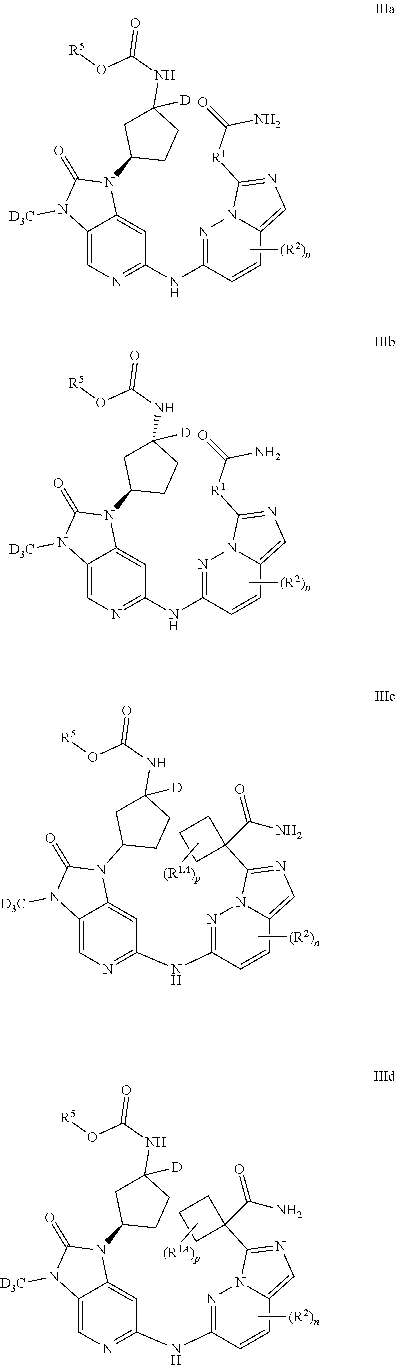

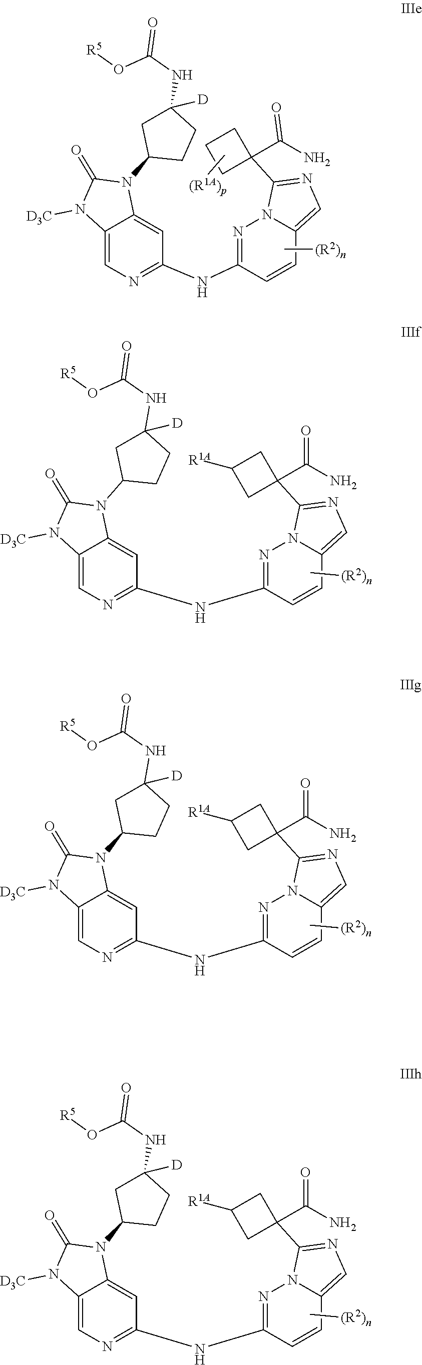

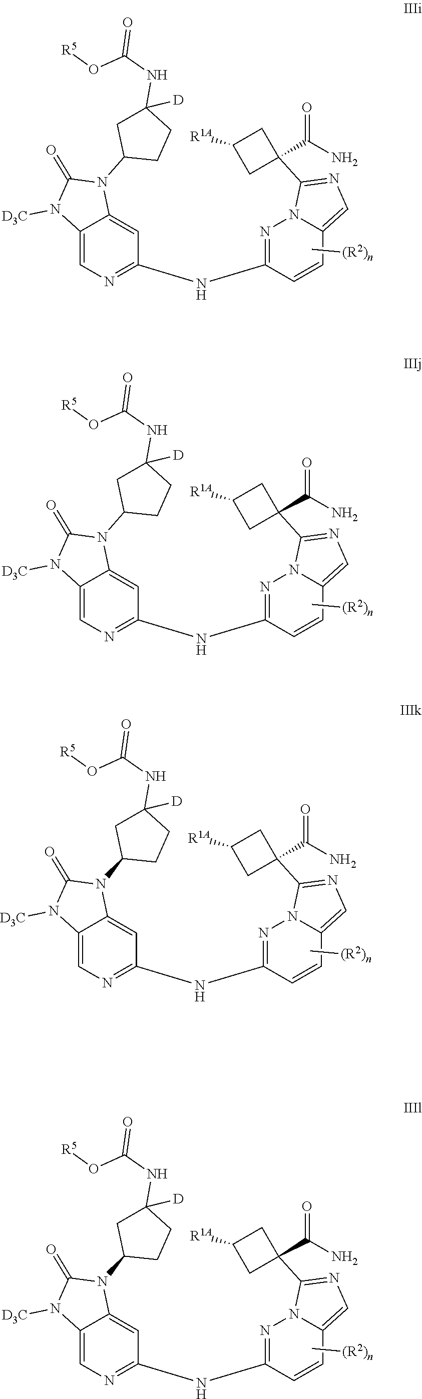

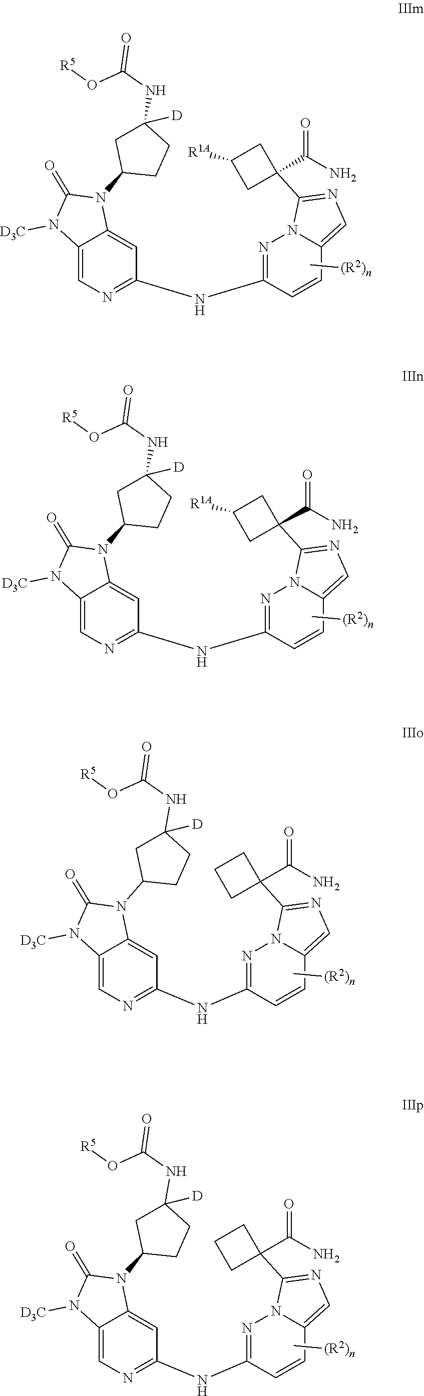

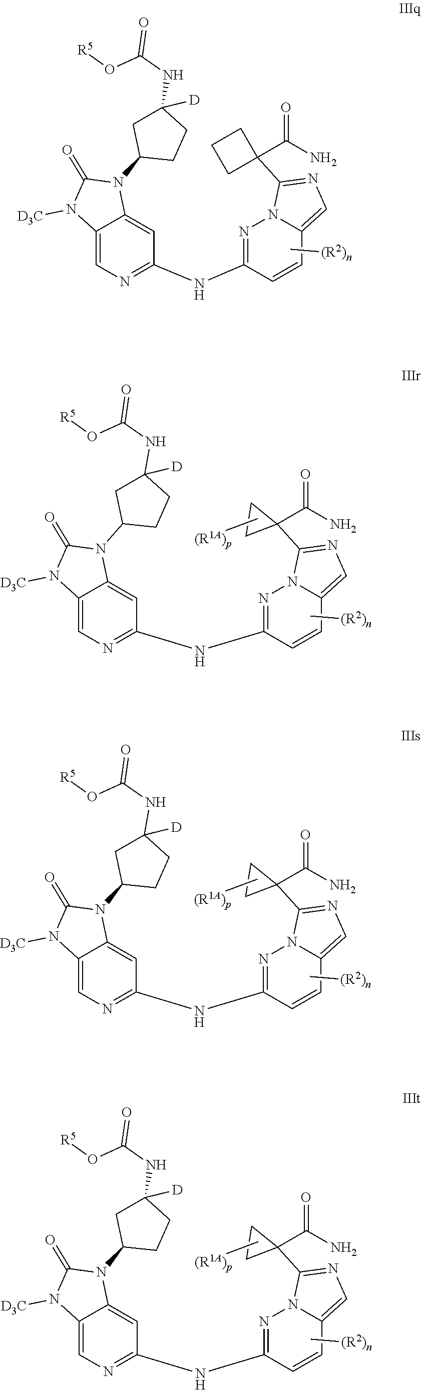

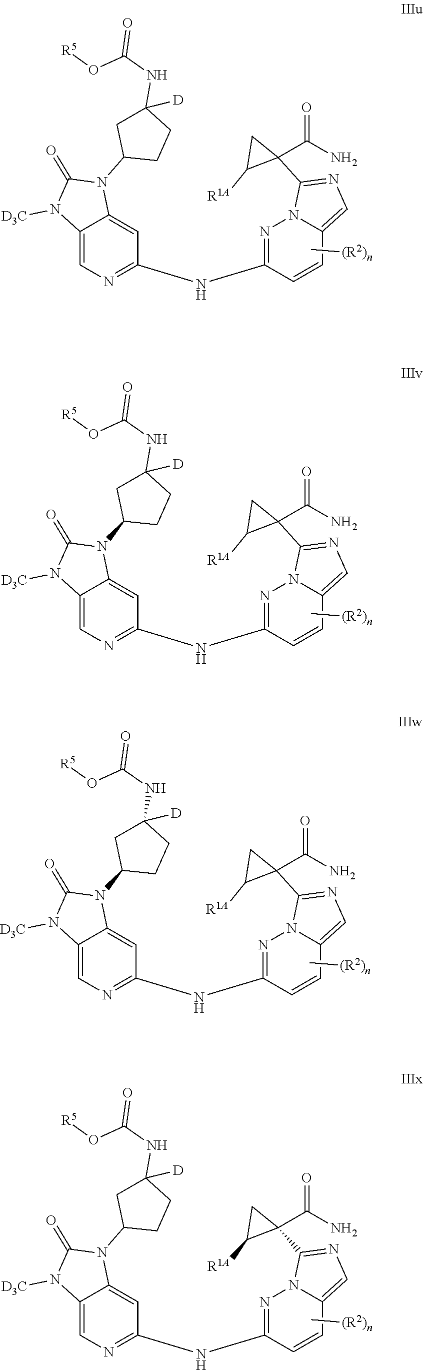

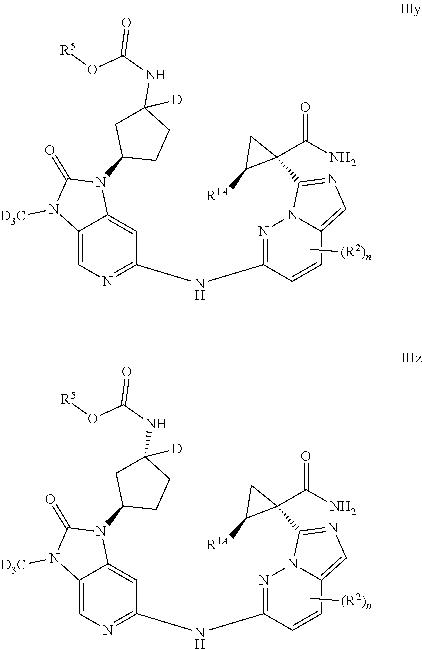

or a pharmaceutically acceptable salt thereof.

In some embodiments, the compound of Formula I or Formula Ia is a compound of Formula IIIa, IIIb, IIIc, IIId, IIIe, IIIf, IIIg, IIIh, IIIi, IIIj, IIIk, IIIl, IIIm, IIIn, IIIo, IIIp, IIIq, IIIr, IIIs, IIIt, IIIu, IIIv, IIIw, IIIx, IIIy, or IIIz, wherein R⁵ is methyl.

In some embodiments, the compound of Formula I or Formula Ia is a compound of Formula IIIa, IIIb, IIIc, IIId, IIIe, IIIf, IIIg, IIIh, IIIi, IIIj, IIIk, IIIl, IIIm, IIIn, IIIo, IIIp, IIIq, IIIr, IIIs, IIIt, IIIu, IIIy, IIIw, IIIx, IIIy, or IIIz, wherein R⁵ is trideuteromethyl.

In some embodiments, the compound of Formula I or Formula Ia is a compound of Formula IV:

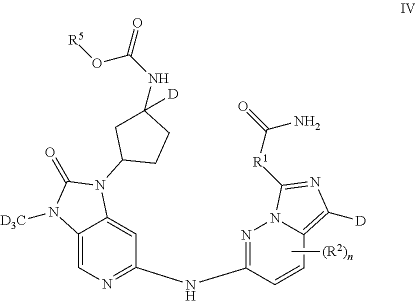

or a pharmaceutically acceptable salt thereof. In some embodiments of Formula IV, R⁵ is methyl. In some embodiments of Formula IV, R⁵ is trideuteromethyl.

In some embodiments, the compound of Formula I or Formula Ia is a compound of Formula IVa, IVb, IVc, IVd, IVe, IVf, IVg, IVh, IVi, IVj, IVk, IVl, IVm, IVn, IVo, IVp, IVq, IVr, IVs, IVt, IVu, IVv, IVw, or IVx:

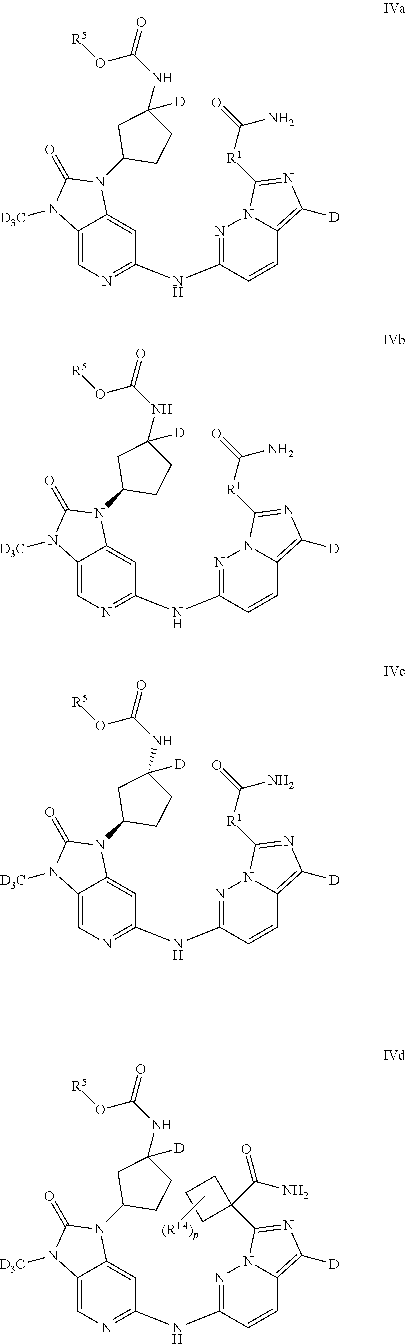

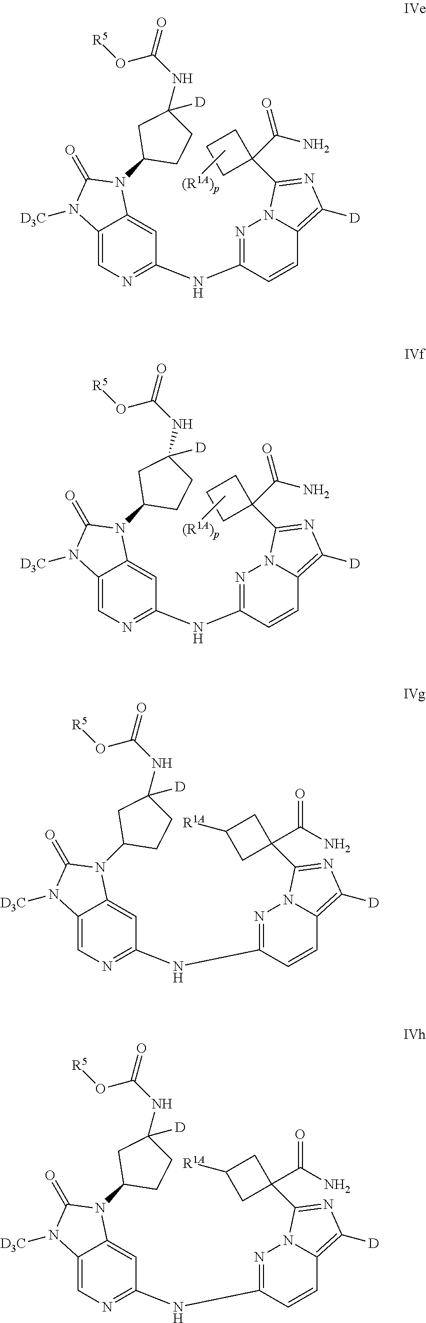

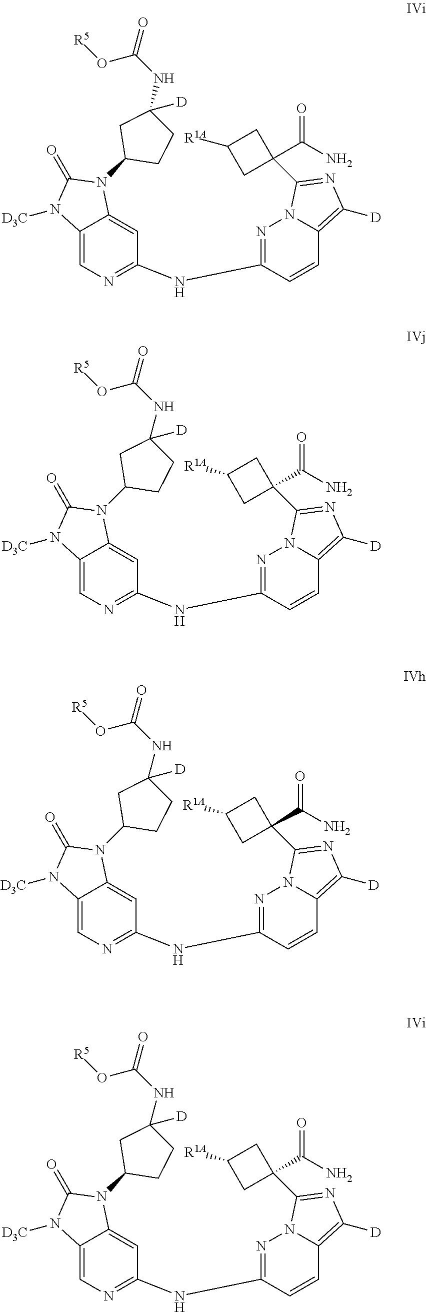

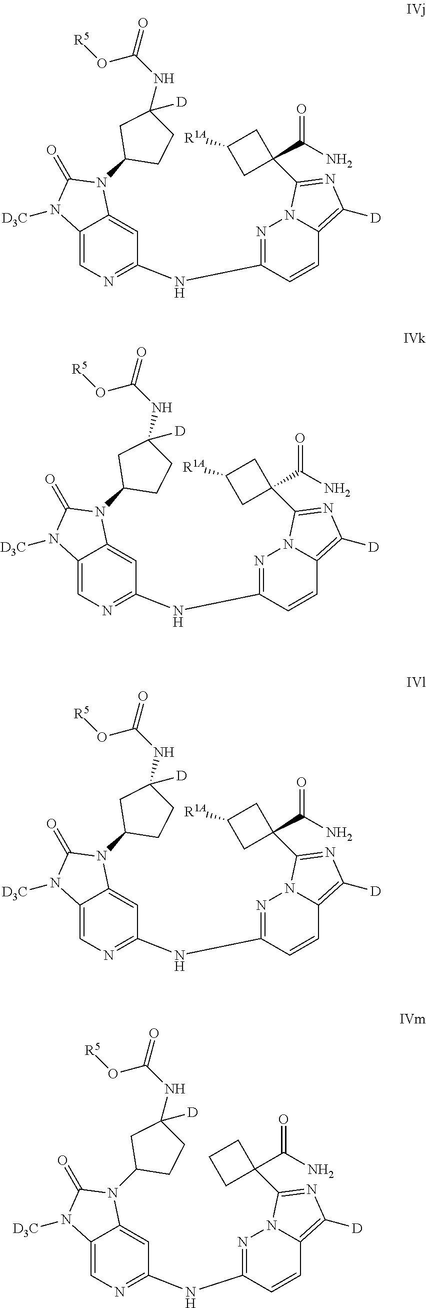

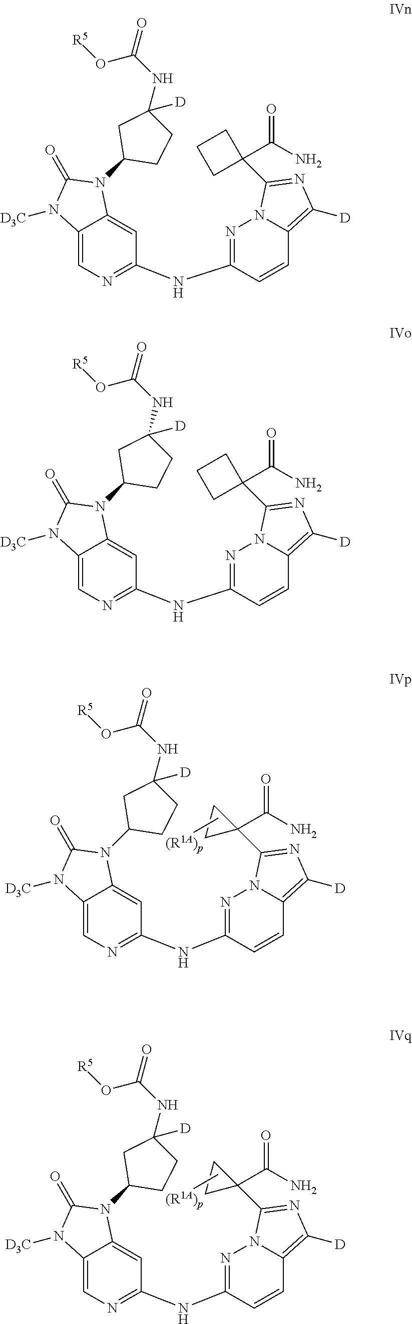

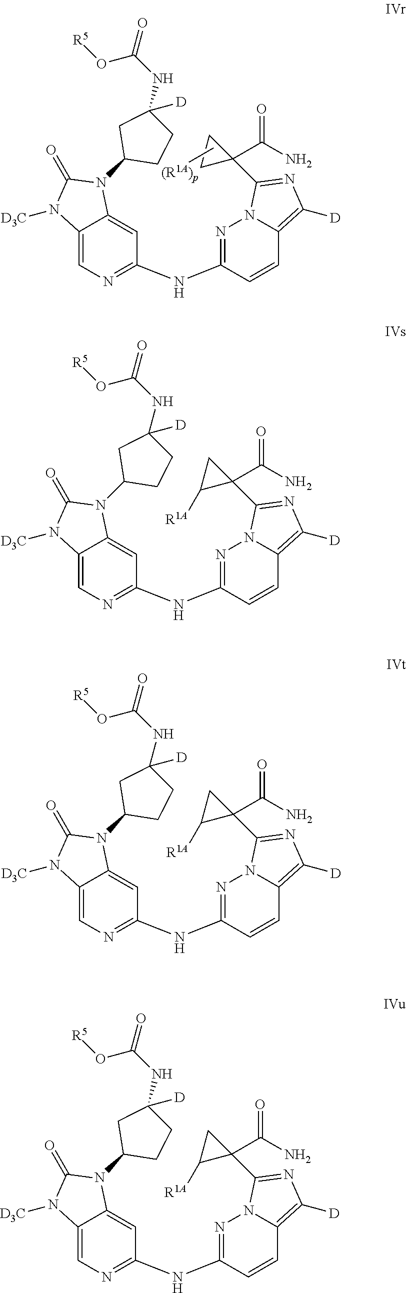

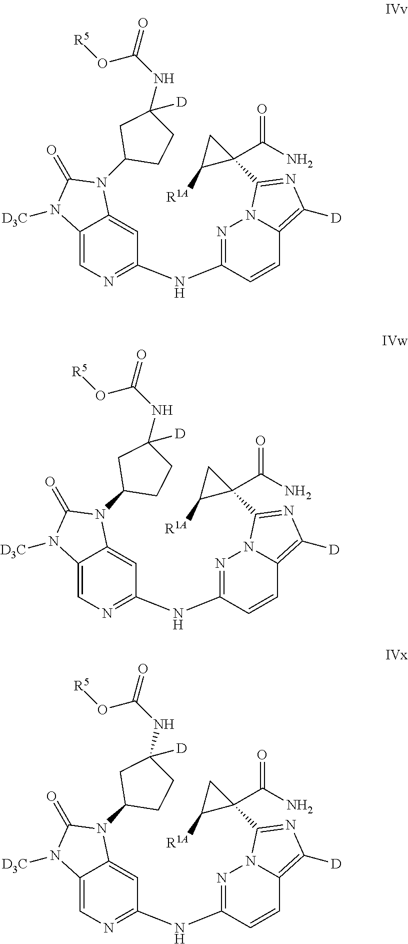

or a pharmaceutically acceptable salt thereof.

In some embodiments, the compound of Formula I or Formula Ia is a compound of Formula IVa, IVb, IVc, IVd, IVe, IVf, IVg, IVh, IVl, IVj, IVk, IVl, IVm, IVn, IVo, IVp, IVq, IVr, IVs, IVt, IVu, IVv, IVw, or IVx, wherein R⁵ is methyl.

In some embodiments, the compound of Formula I or Formula Ia is a compound of Formula IVa, IVb, IVc, IVd, IVe, IVf, IVg, IVh, IVl, IVj, IVk, IVl, IVm, IVn, IVo, IVp, IVq, IVr, IVs, IVt, IVu, IVv, IVw, or IVx, wherein R⁵ is trideuteromethyl.

In some embodiments, the compound of Formula I or Formula Ia is a compound of Formula V:

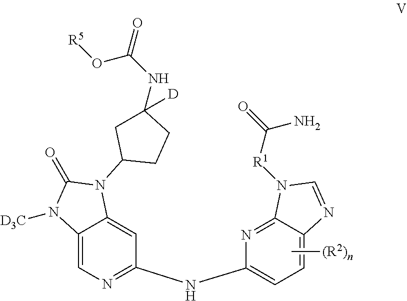

or a pharmaceutically acceptable salt thereof. In some embodiments of Formula V, R⁵ is methyl. In some embodiments of Formula V, R⁵ is trideuteromethyl.

In some embodiments, the compound of Formula I or Formula Ia is a compound of Formula Va, Vb, Vc, Vd, Ve, Vf, Vg, Vh, Vi, Vj, Vk, Vl, Vm, Vn, Vo, Vp, or Vq:

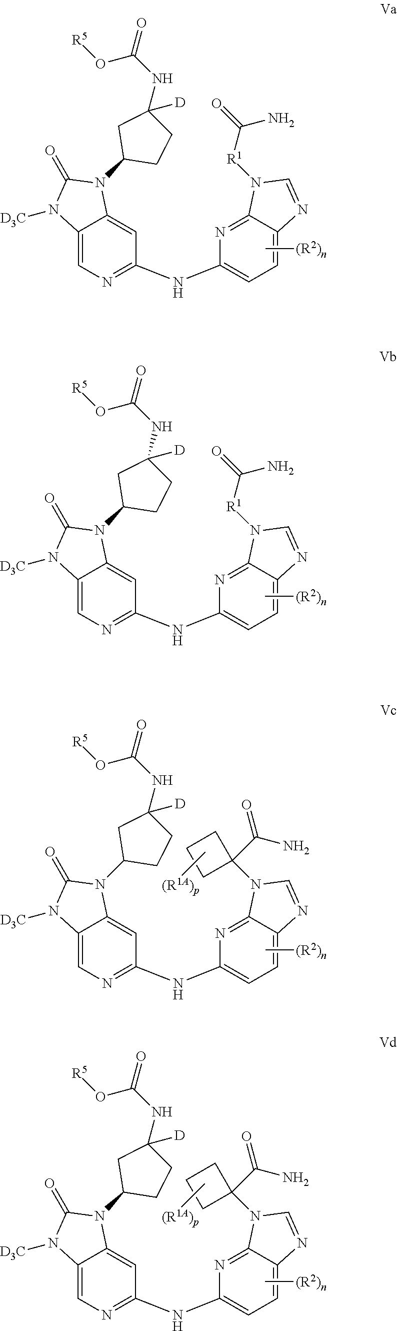

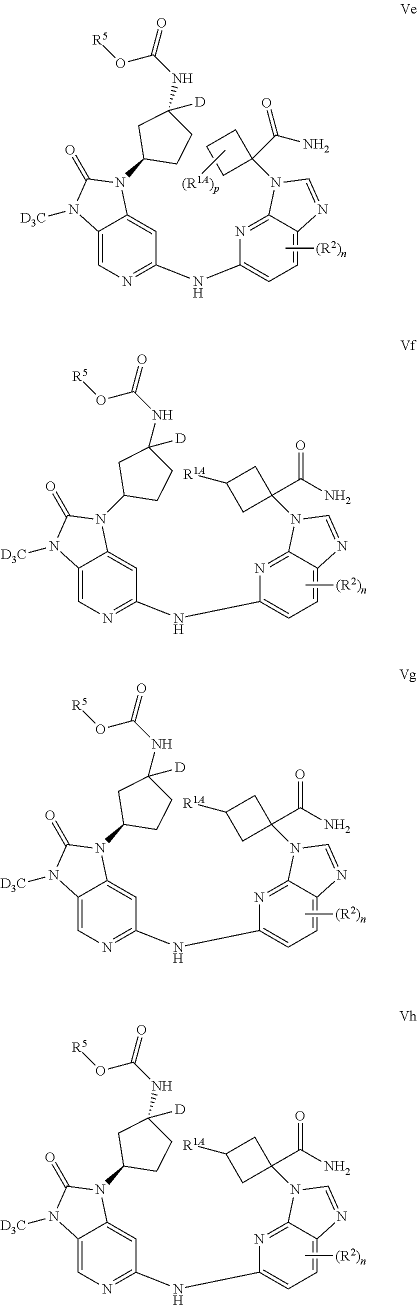

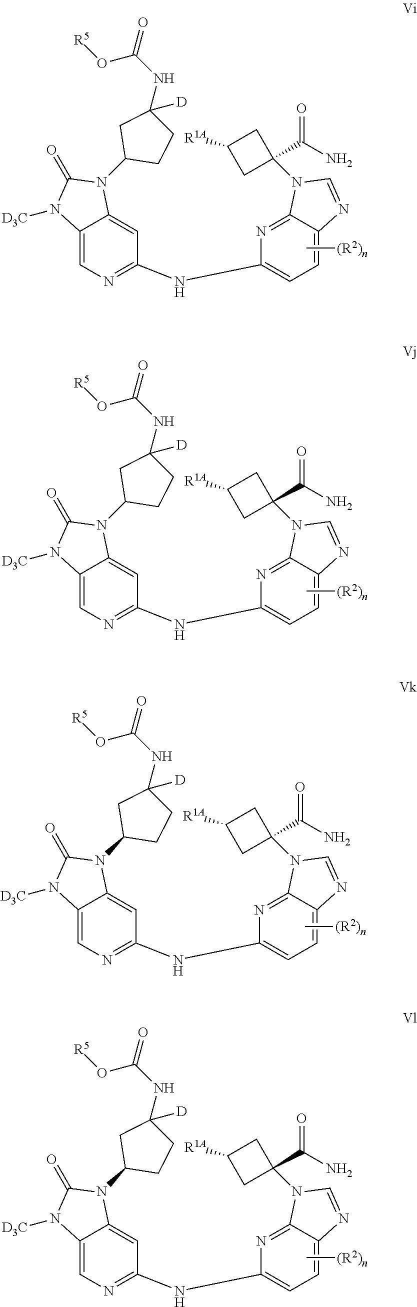

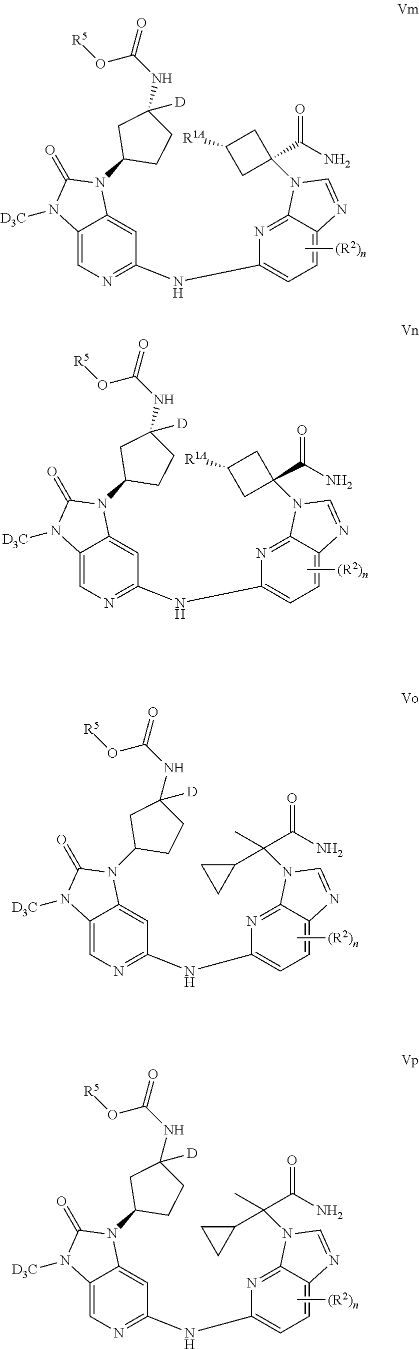

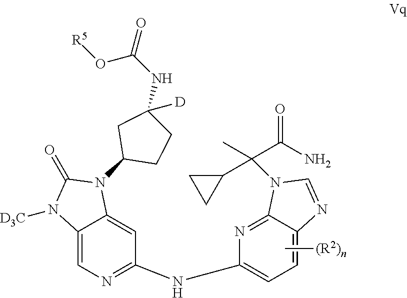

or a pharmaceutically acceptable salt thereof.

In some embodiments, the compound of Formula I or Formula Ia is a compound of Formula Va, Vb, Vc, Vd, Ve, Vf, Vg, Vh, Vi, Vj, Vk, Vl, Vm, Vn, Vo, Vp, or Vq, R⁵ is methyl.

In some embodiments, the compound of Formula I or Formula Ia is a compound of Formula Va, Vb, Vc, Vd, Ve, Vf, Vg, Vh, Vi, Vj, Vk, Vl, Vm, Vn, Vo, Vp, or Vq, R⁵ is trideuteromethyl.

In some embodiments, the compound of Formula I or Formula Ia is a compound of Formula VI:

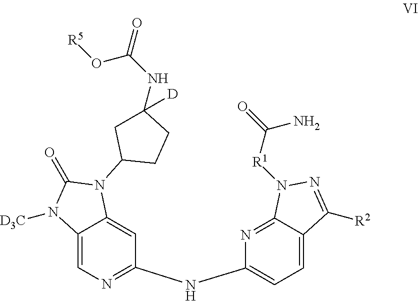

or a pharmaceutically acceptable salt thereof. In some embodiments of Formula VI, R⁵ is methyl. In some embodiments of Formula VI, R⁵ is trideuteromethyl.

In some embodiments, the compound of Formula I or Formula Ia is a compound of Formula VIa, VIb, VIc, VId, VIe, VIf, VIg, VIh, VIi, VIj, VIk, VIl, VIm, VIn, VIo, VIp, VIq, VIr, VIs, VIt, VIu, VIv, VIw, VIx, VIy, VIz, VIaa, VIab, VIac, VIad, VIae, VIaf, VIag, VIah, VIai, VIaj, VIak, VIal, VIam, VIan, VIao, VIap, VIaq, or VIar:

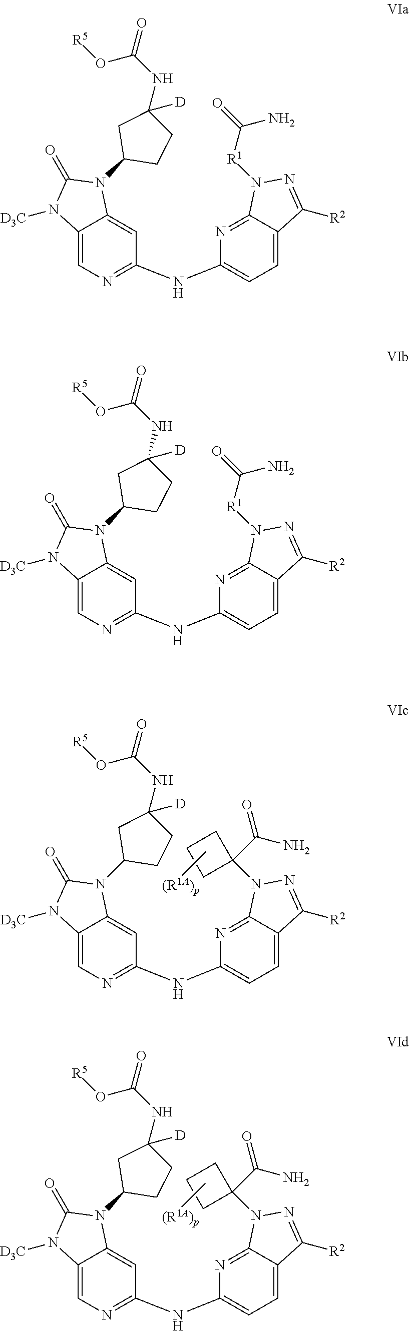

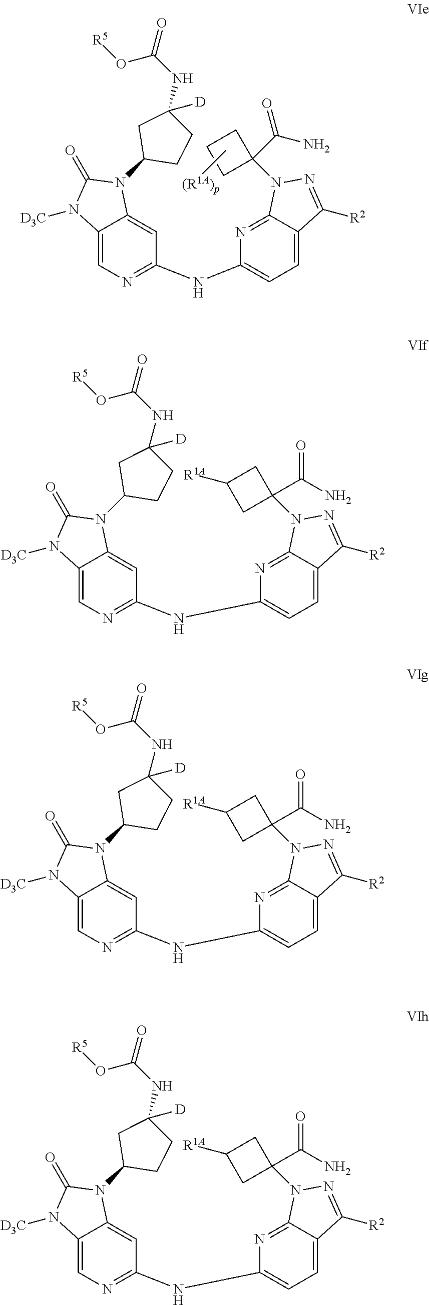

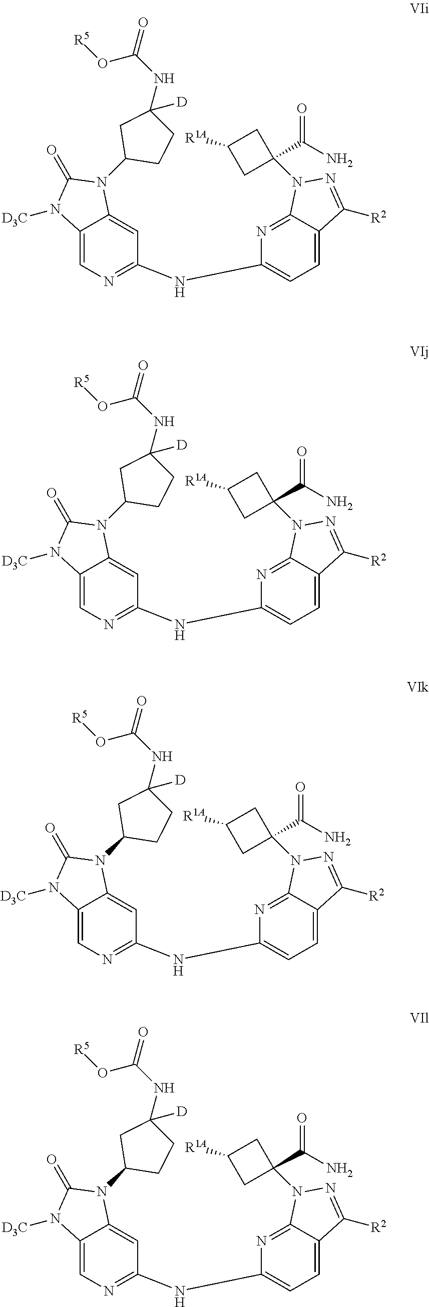

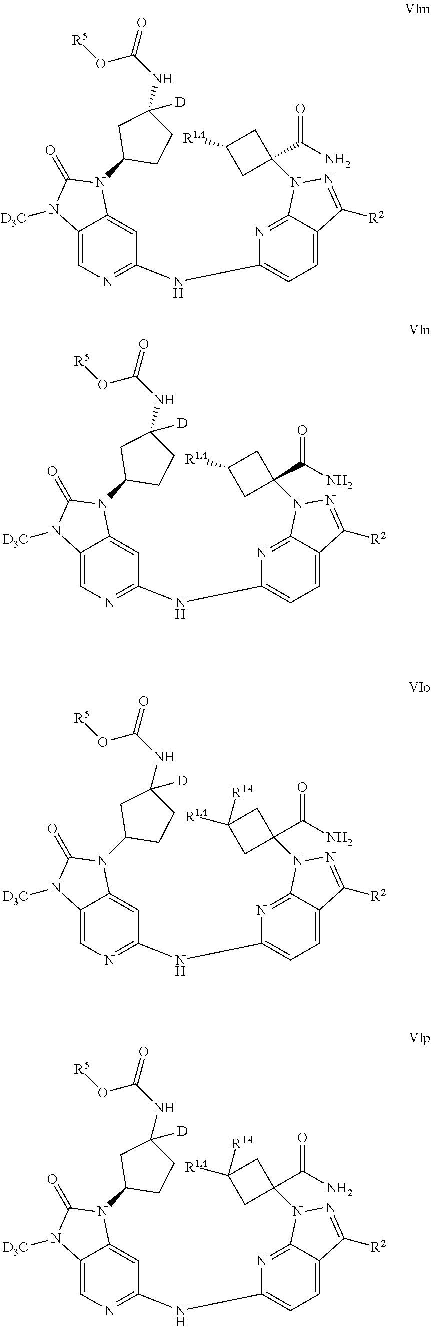

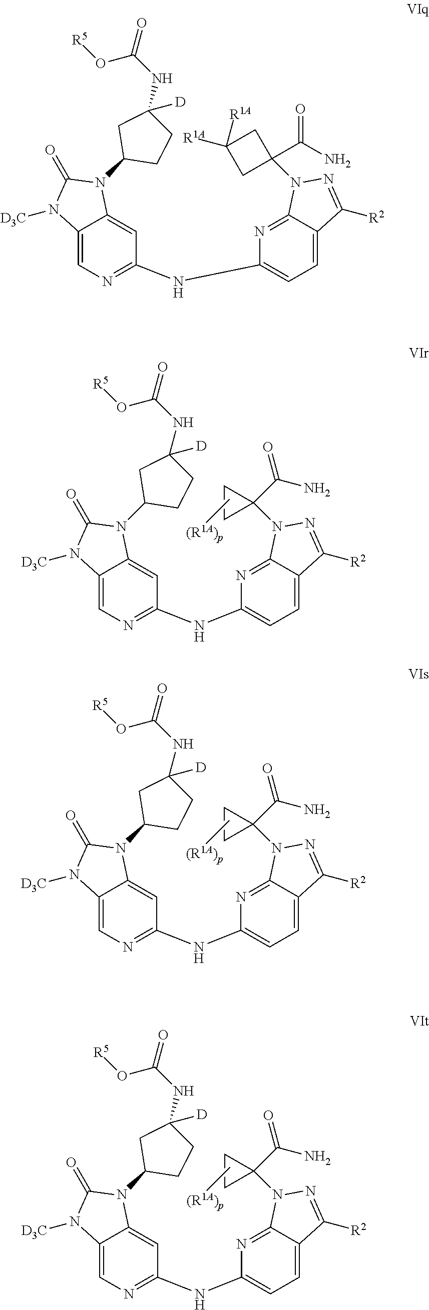

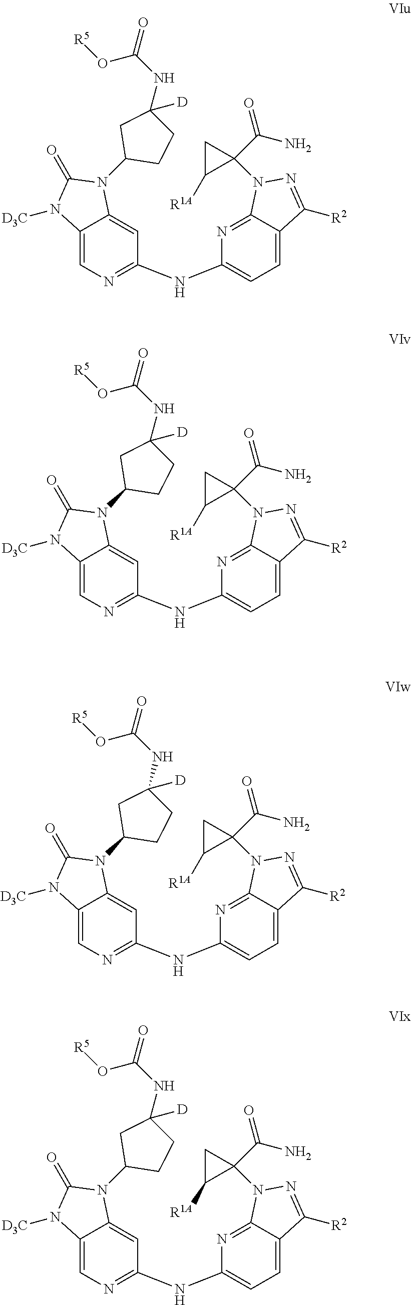

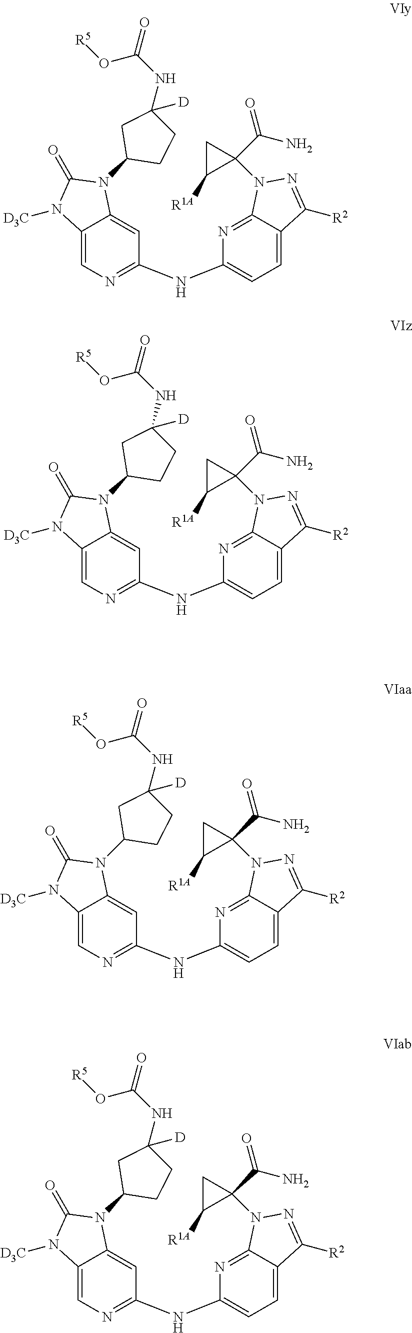

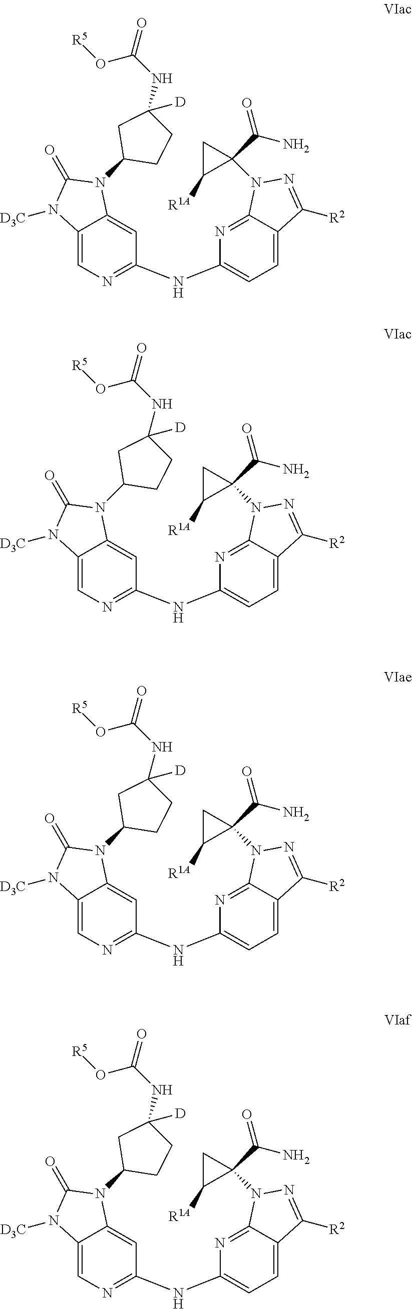

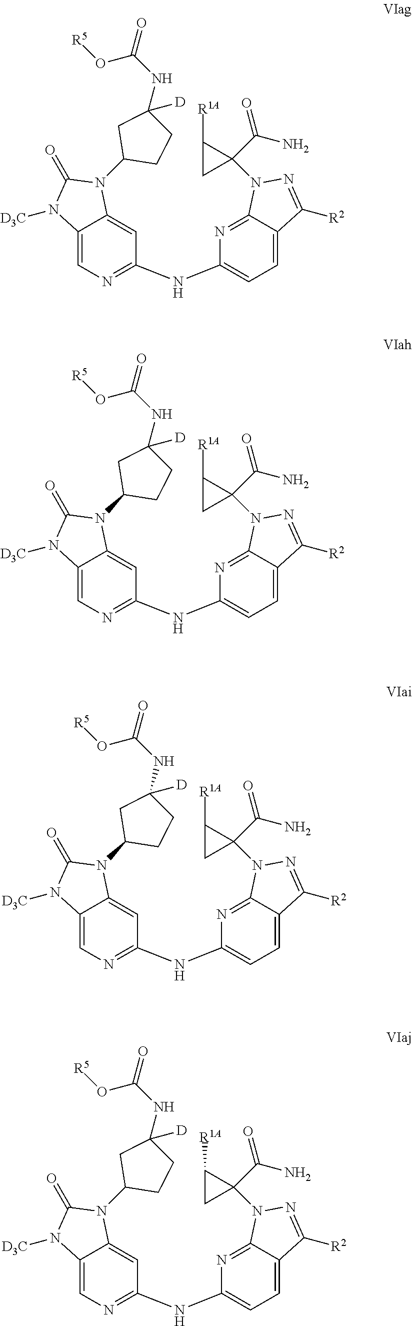

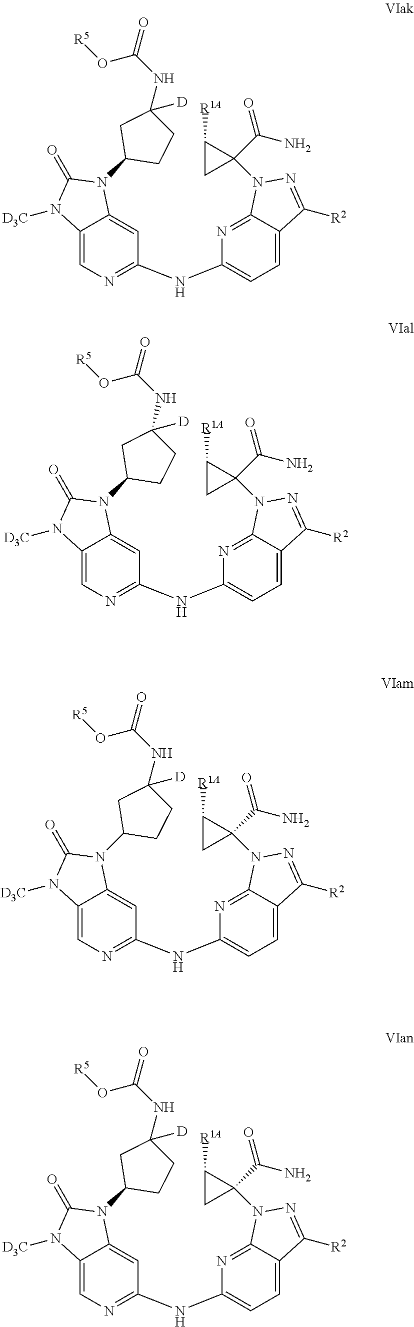

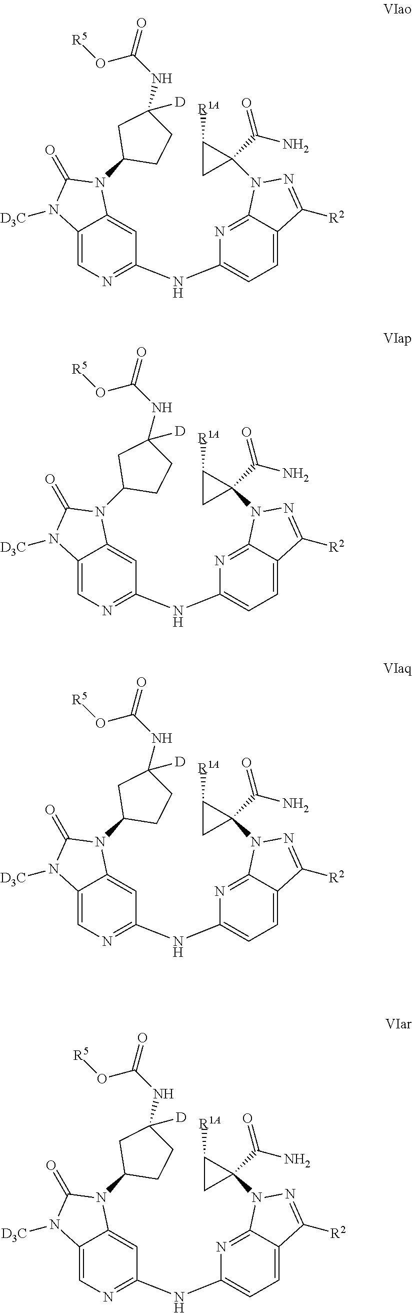

or a pharmaceutically acceptable salt thereof.

In some embodiments, the compound of Formula I or Formula Ia is a compound of Formula VIa, VIb, VIc, VId, VIe, VIf, VIg, VIh, ViI, VIj, VIk, VIl, VIm, VIn, VIo, VIp, VIq, VIr, VIs, VIt, VIu, VIv, VIw, VIx, VIy, VIz, VIaa, VIab, VIac, VIad, VIae, VIaf, VIag, VIah, VIai, VIaj, VIak, VIal, VIam, VIan, VIao, VIap, VIaq, or VIar, wherein R⁵ is methyl.

In some embodiments, the compound of Formula I or Formula Ia is a compound of Formula VIa, VIb, VIc, VId, VIe, VIf, VIg, VIh, ViI, VIj, VIk, VIl, VIm, VIn, VIo, VIp, VIq, VIr, VIs, VIt, VIu, VIv, VIw, VIx, VIy, VIz, VIaa, VIab, VIac, VIad, VIae, VIaf, VIag, VIah, VIai, VIaj, VIak, VIal, VIam, VIan, VIao, VIap, VIaq, or VIar, wherein R⁵ is trideuteromethyl.

In some embodiments, the compound of Formula I or Formula Ia is a compound of Formula VII:

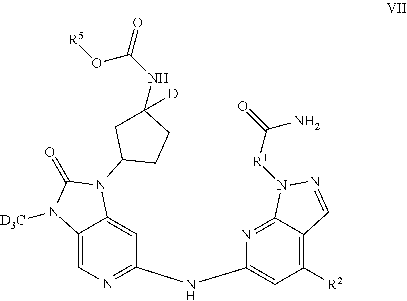

or a pharmaceutically acceptable salt thereof. In some embodiments of Formula VII, R⁵ is methyl. In some embodiments of Formula VII, R⁵ is trideuteromethyl.

In some embodiments, the compound of Formula I or Formula Ia is a compound of Formula VIIa, VIIb, VIIc, VIId, VIIe, VIIf, VIIg, VIIh, VIIi, VIIj, VIIk, VIIl, VIIm, VIIn, VIIo, VIIp, VIIq, VIIr, VIIs, VIIt, VIIu, VIIv, VIIw, VIIx, VIIy, VIIz, VIIaa, VIIab, VIIac, VIIad, VIIae, or VIIaf:

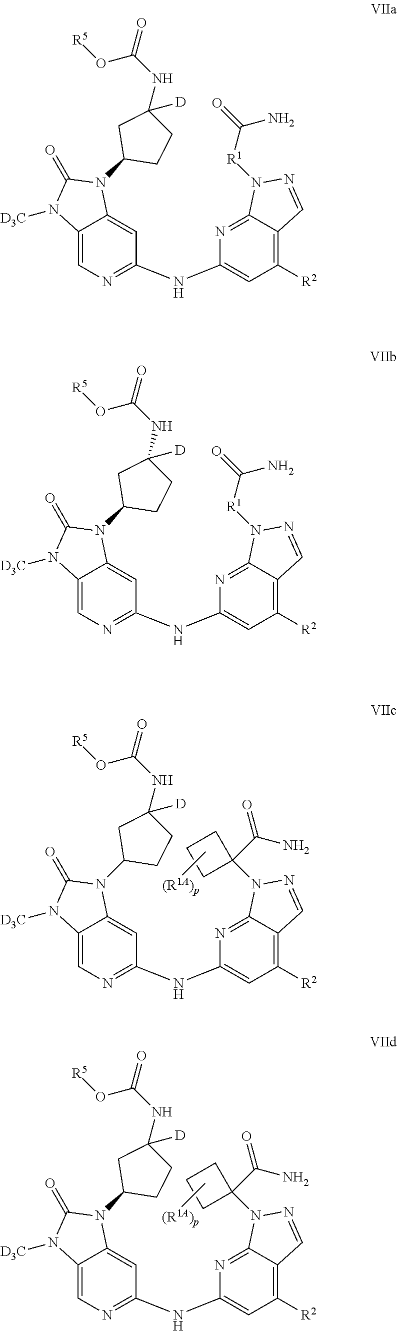

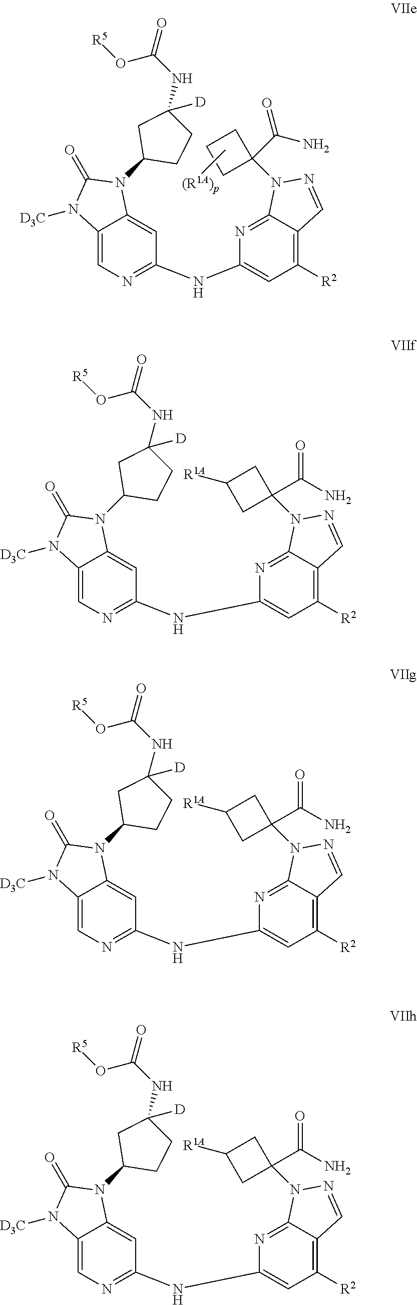

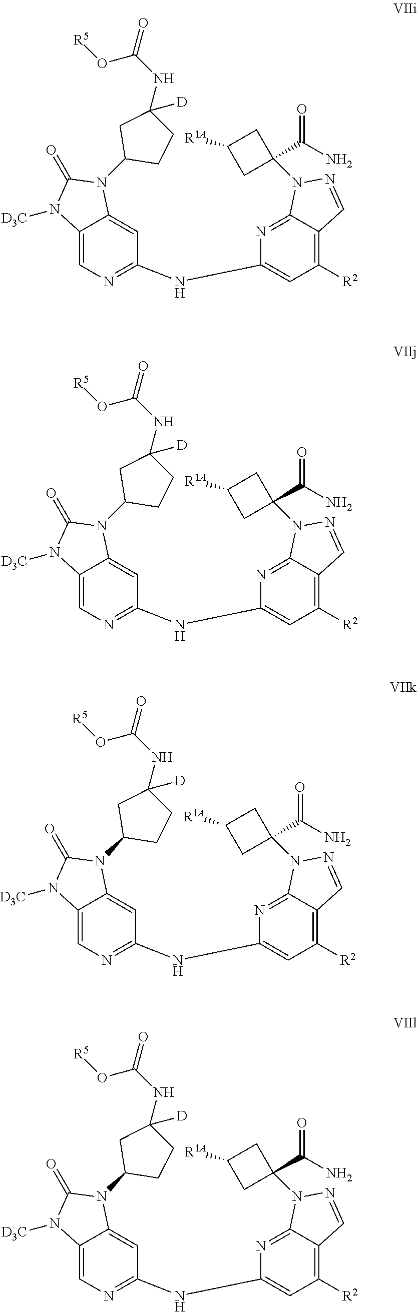

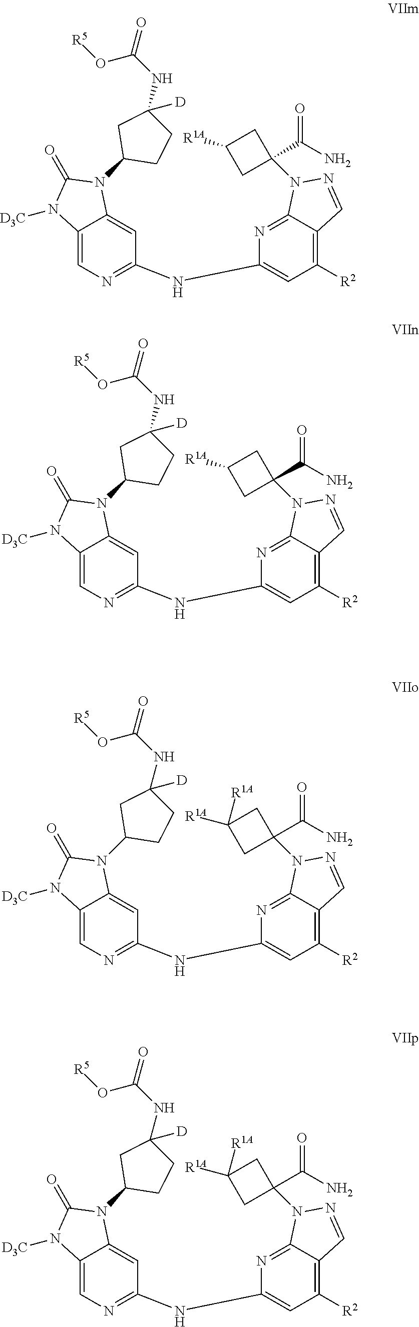

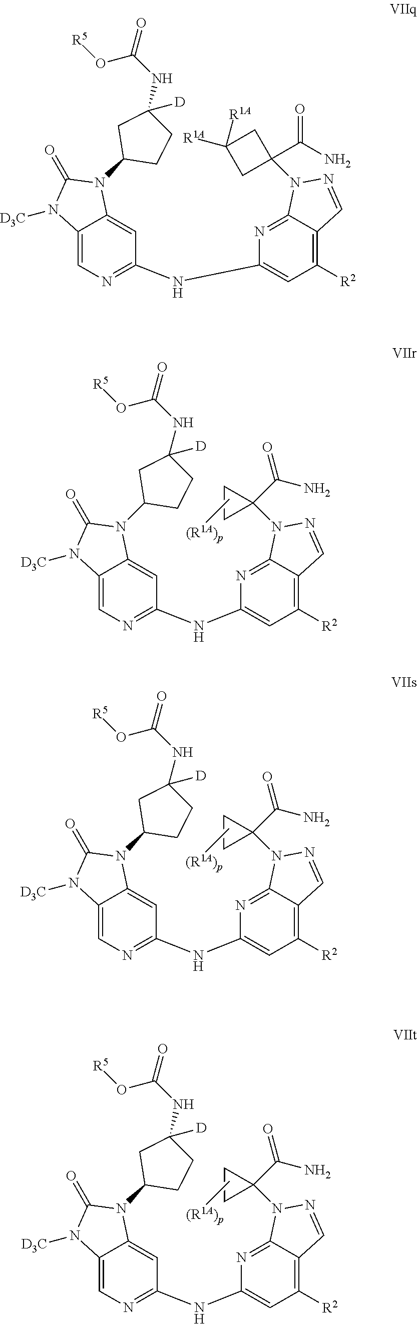

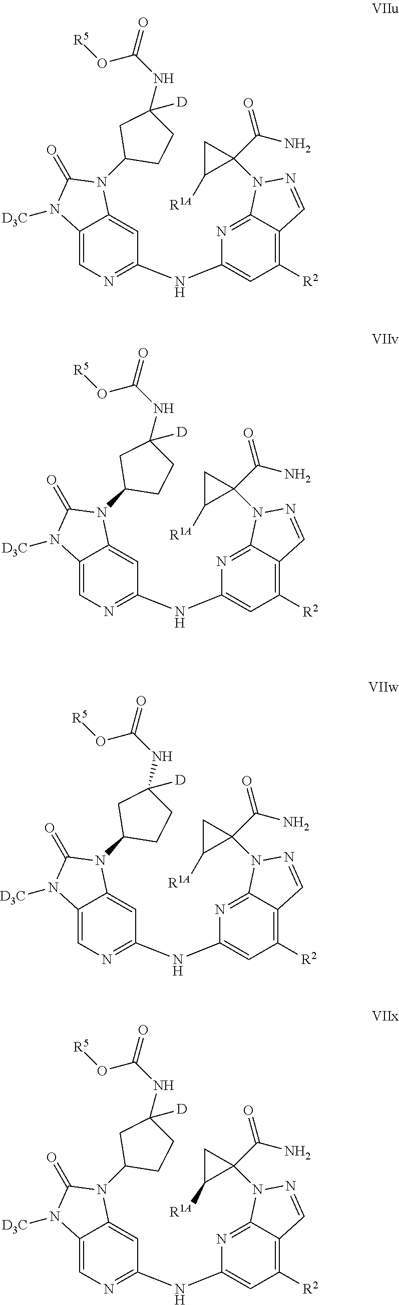

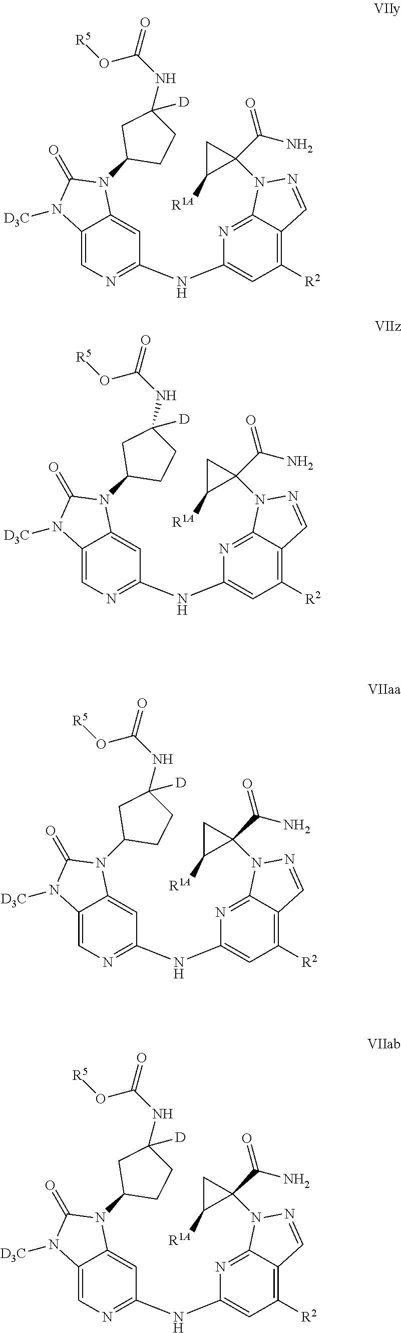

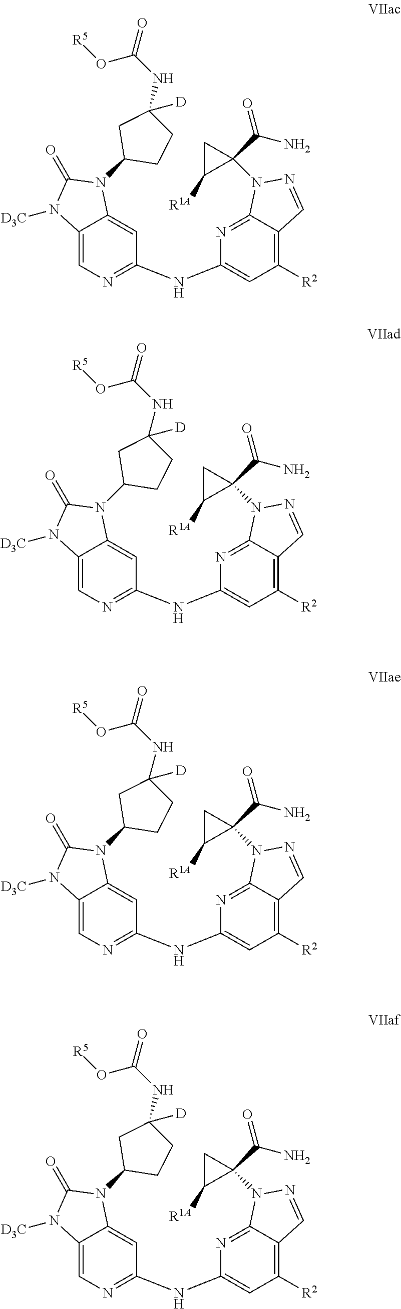

or a pharmaceutically acceptable salt thereof.

In some embodiments, the compound of Formula I or Formula Ia is a compound of Formula VIIa, VIIb, VIIc, VIId, VIIe, VIIf, VIIg, VIIh, VIIi, VIIj, VIIk, VIIl, VIIm, VIIn, VIIo, VIIp, VIIq, VIIr, VIIs, VIIt, VIIu, VIIv, VIIw, VIIx, VIIy, VIIz, VIIaa, VIIab, VIIac, VIIad, VIIae, or VIIaf, wherein R⁵ is methyl.

In some embodiments, the compound of Formula I or Formula Ia is a compound of Formula VIIa, VIIb, VIIc, VIId, VIIe, VIIf, VIIg, VIIh, VIIi, VIIj, VIIk, VIIl, VIIm, VIIn, VIIo, VIIp, VIIq, VIIr, VIIs, VIIt, VIIu, VIIv, VIIw, VIIx, VIIy, VIIz, VIIaa, VIIab, VIIac, VIIad, VIIae, or VIIaf, wherein R⁵ is trideuteromethyl.

In some embodiments, the compound of Formula I or Formula Ia is a compound of Formula VIII:

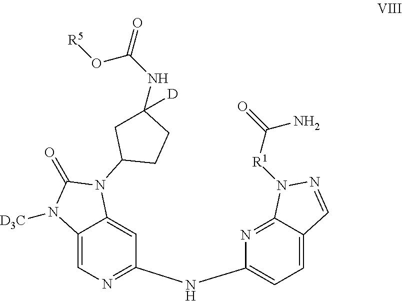

or a pharmaceutically acceptable salt thereof. In some embodiments of Formula VIII, R⁵ is methyl. In some embodiments of Formula VIII, R⁵ is trideuteromethyl.

In some embodiments, the compound of Formula I or Formula Ia is a compound of Formula VIIIa, VIIIb, VIIIc, VIIId, VIIIe, VIIIf, VIIIg, VIIIh, VIIIi, VIIIj, VIIIk, VIIIl, VIIIm, VIIIn, VIIIo, VIIIp, VIIIq, VIIIr, VIIIs, VIIIt, VIIIu, VIIIv, VIIIw, VIIIx, VIIIy, VIIIz, VIIIaa, VIIIab, VIIIac, VIIIad, VIIIae, VIIIaf:

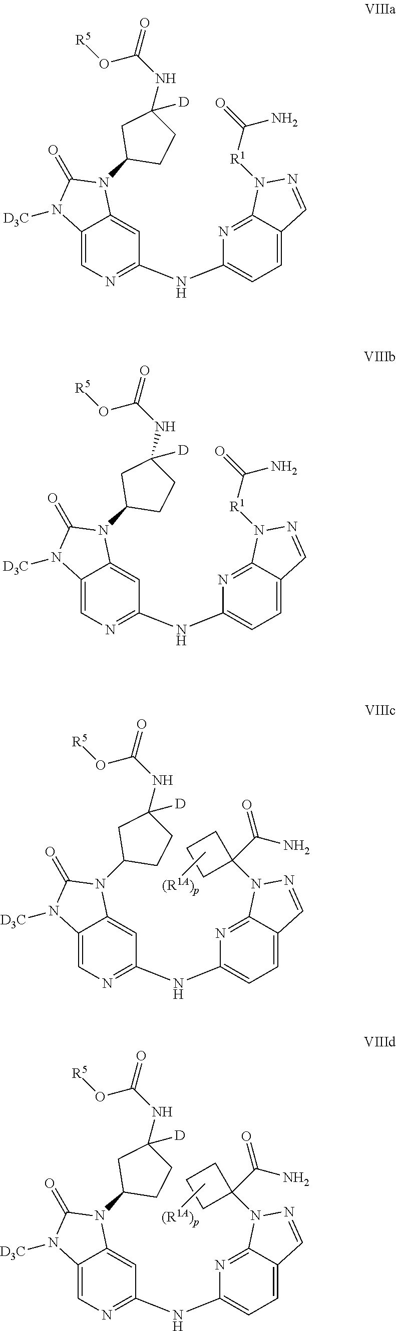

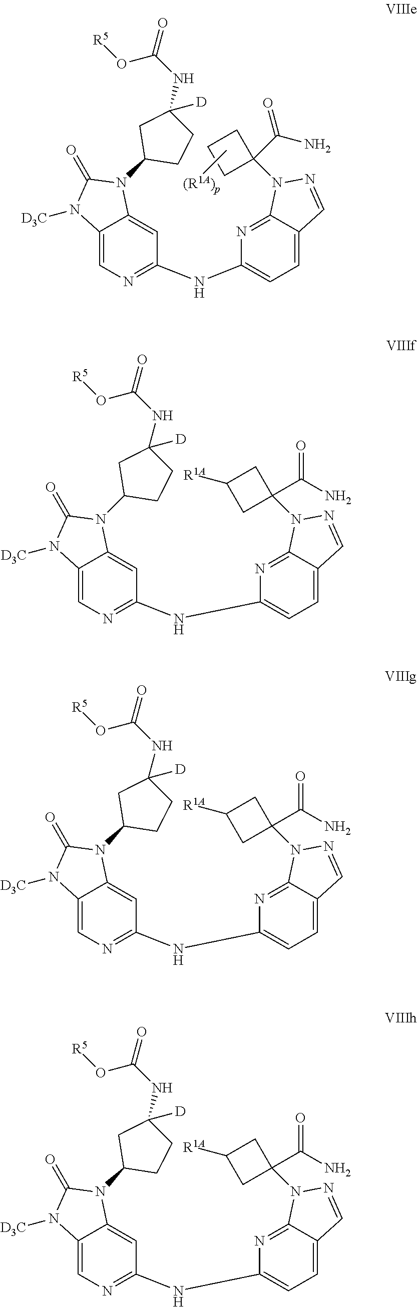

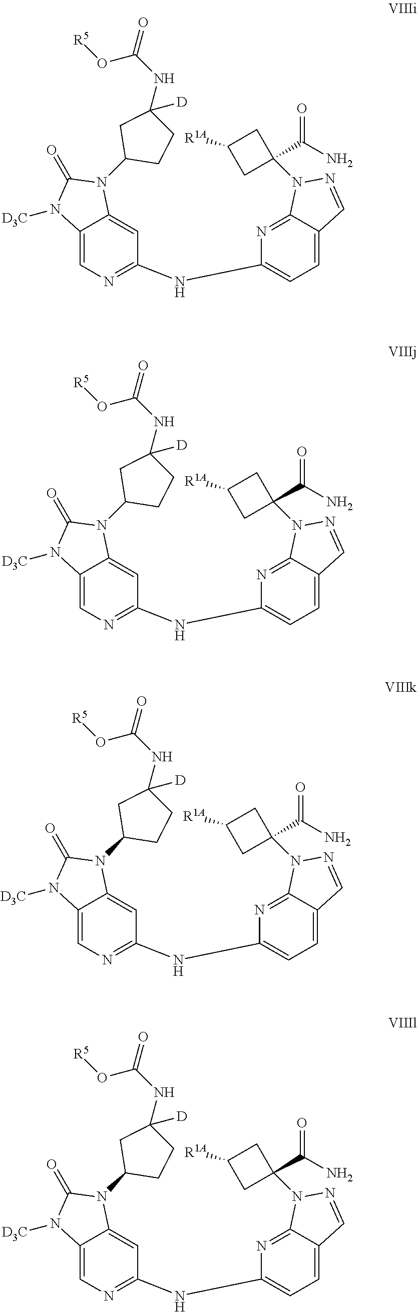

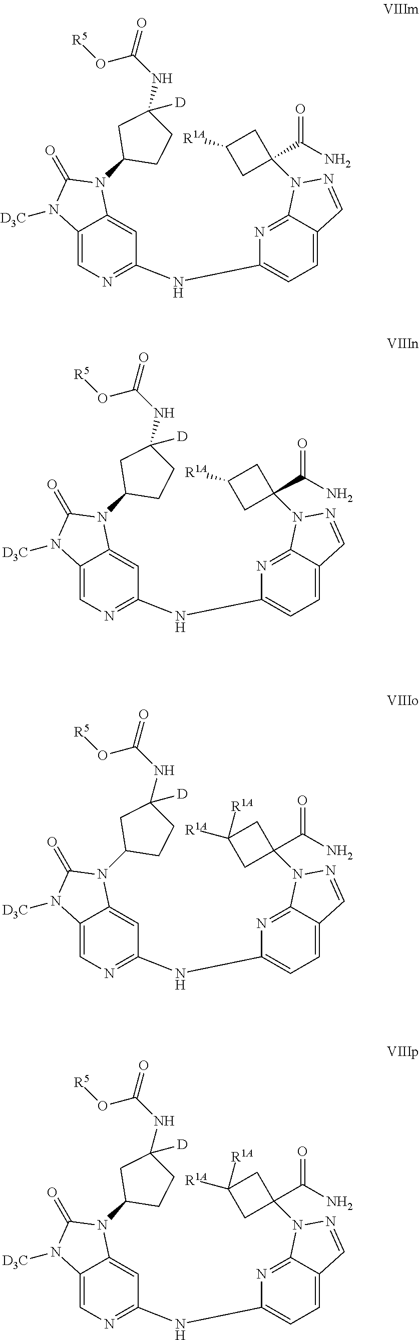

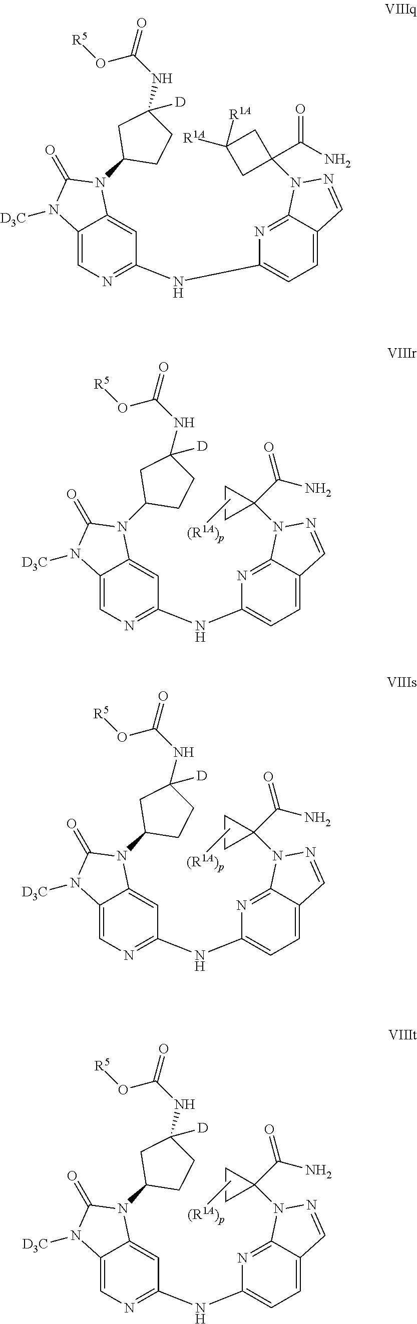

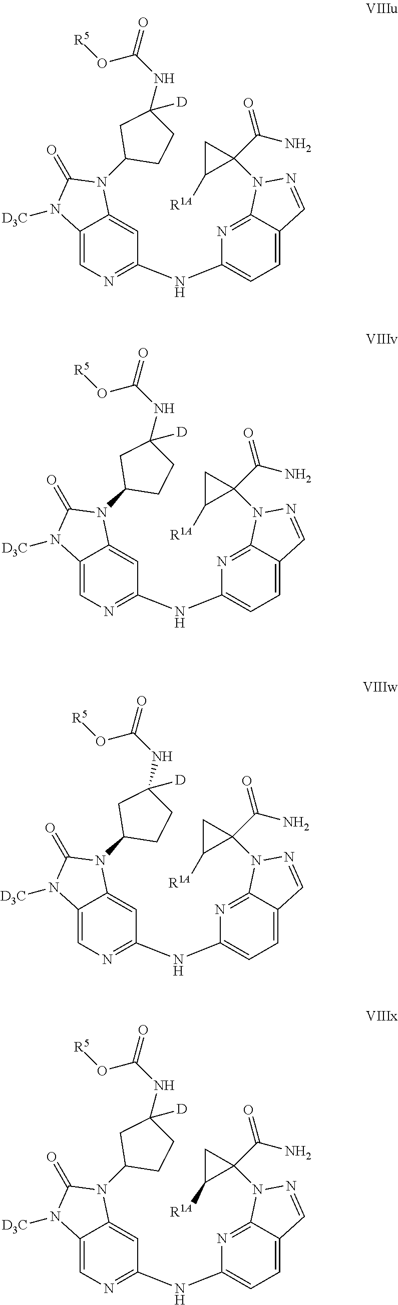

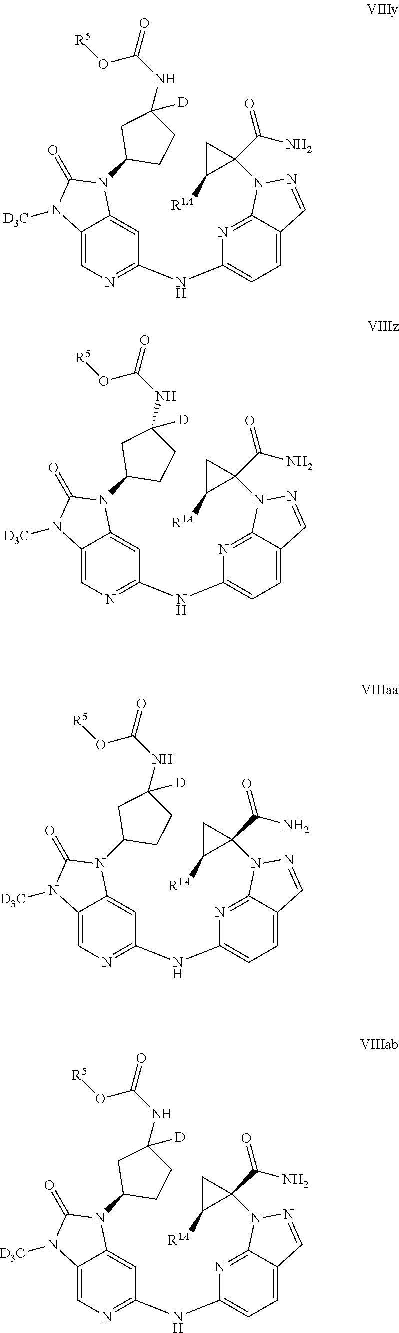

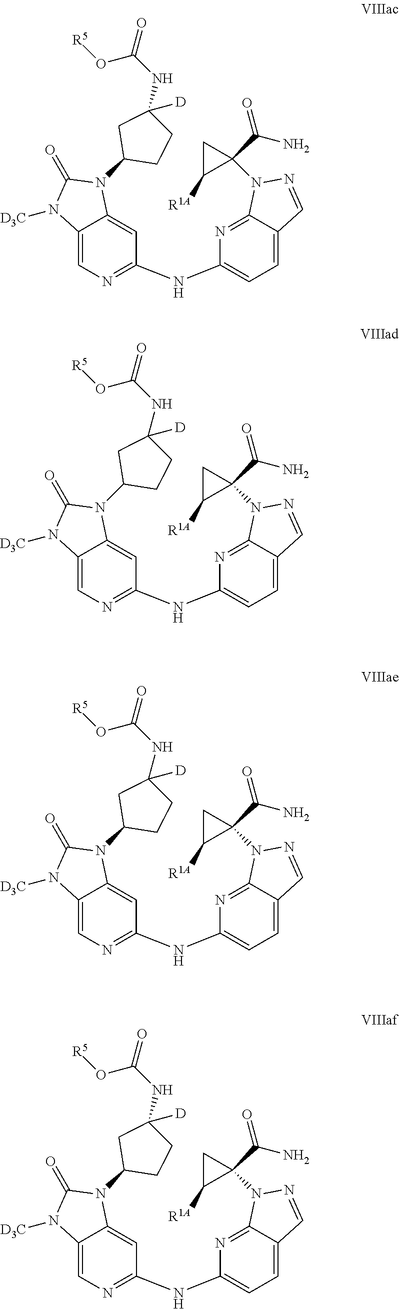

or a pharmaceutically acceptable salt thereof.

In some embodiments, the compound of Formula I or Formula Ia is a compound of Formula VIIIa, VIIIb, VIIIc, VIIId, VIIIe, VIIIf, VIIIg, VIIIh, VIIII, VIIIj, VIIIk, VIIIl, VIIIm, VIIIn, VIIIo, VIIIp, VIIIq, VIIIr, VIIIs, VIIIt, VIIIu, VIIIv, VIIIw, VIIIx, VIIIy, VIIIz, VIIIaa, VIIIab, VIIIac, VIIIad, VIIIae, VIIIaf, wherein R⁵ is methyl.

In some embodiments, the compound of Formula I or Formula Ia is a compound of Formula VIIIa, VIIIb, VIIIc, VIIId, VIIIe, VIIIf, VIIIg, VIIIh, VIIII, VIIIj, VIIIk, VIIIl, VIIIm, VIIIn, VIIIo, VIIIp, VIIIq, VIIIr, VIIIs, VIIIt, VIIIu, VIIIv, VIIIw, VIIIx, VIIIy, VIIIz, VIIIaa, VIIIab, VIIIac, VIIIad, VIIIae, VIIIaf, wherein R⁵ is trideuteromethyl.

In some embodiments, the compound of Formula I or Formula Ia is a compound of Formula IX:

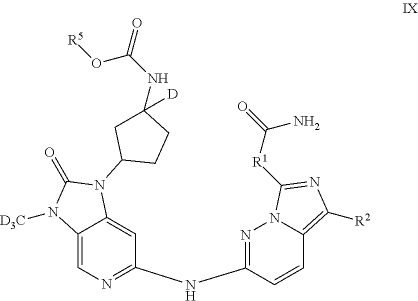

or a pharmaceutically acceptable salt thereof. In some embodiments of Formula IX, R⁵ is methyl. In some embodiments of Formula IX, R⁵ is trideuteromethyl.

In some embodiments, the compound of Formula I or Formula Ia is a compound of Formula IXa, IXb, IXc, IXd, IXe, IXf, IXg, IXh, IXi, IXj, IXk, IXl, IXm, IXn, IXo, IXp, IXq, IXr, IXs, IXt, IXu, IXv, IXw, IXx, IXy, or IXz:

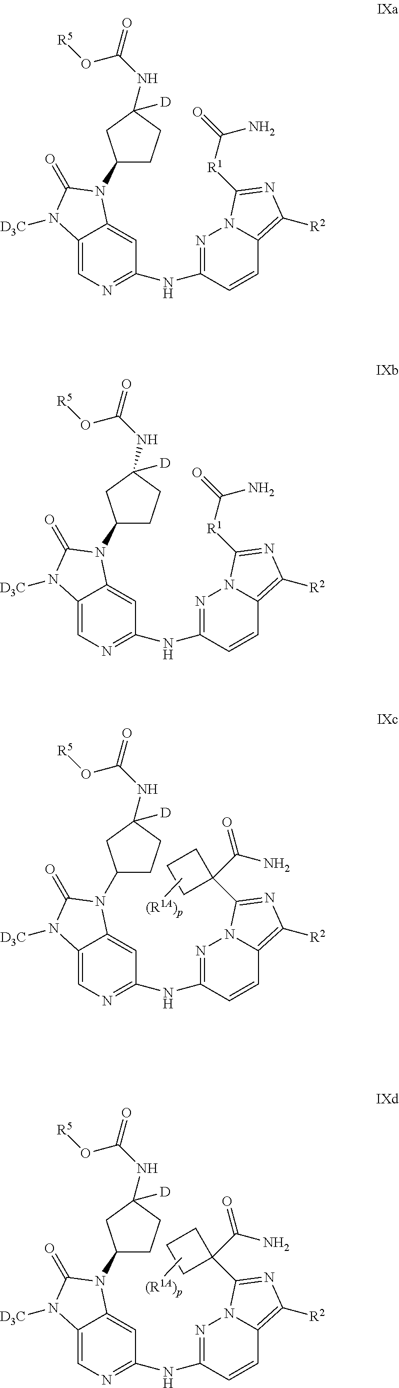

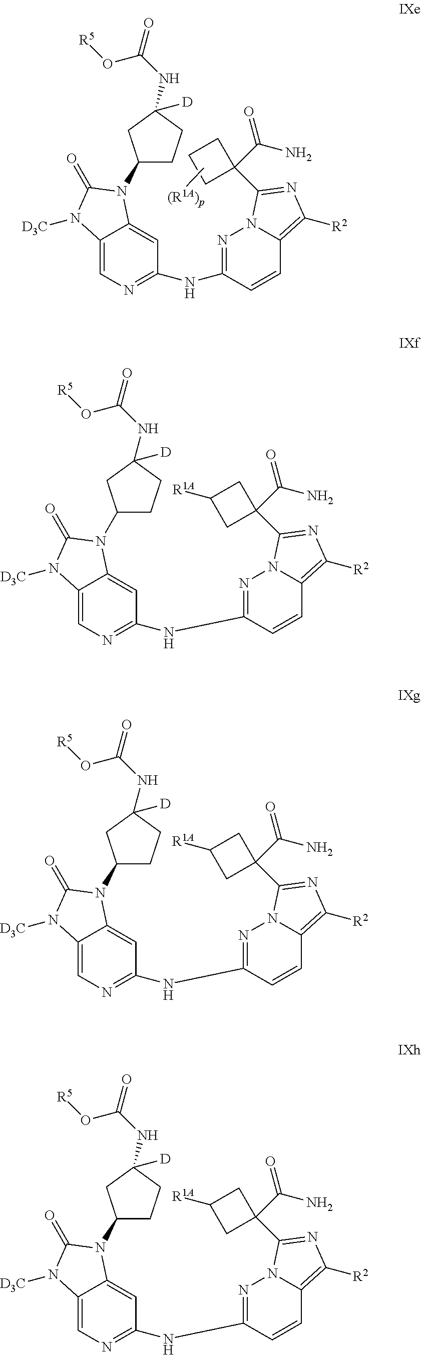

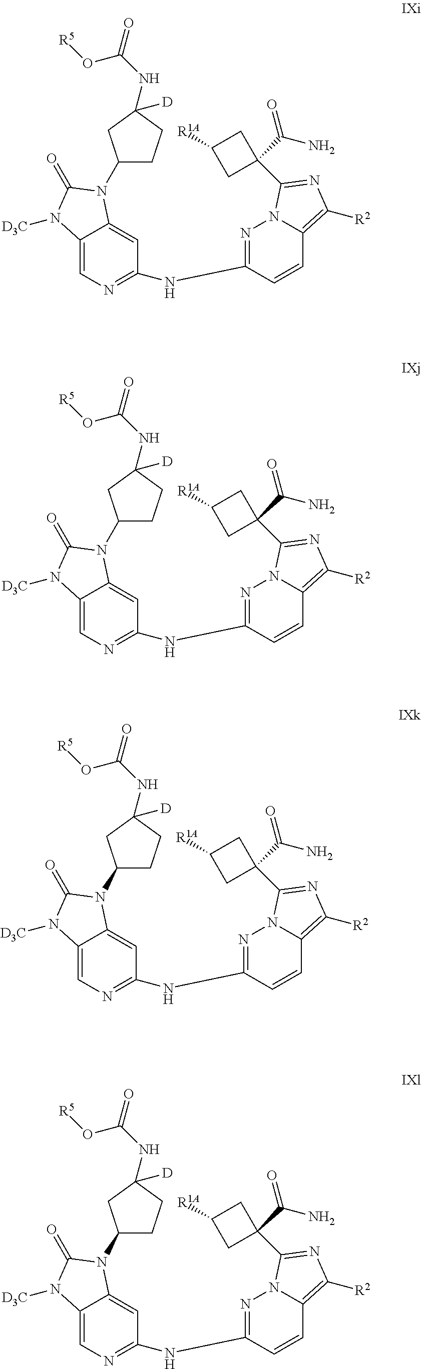

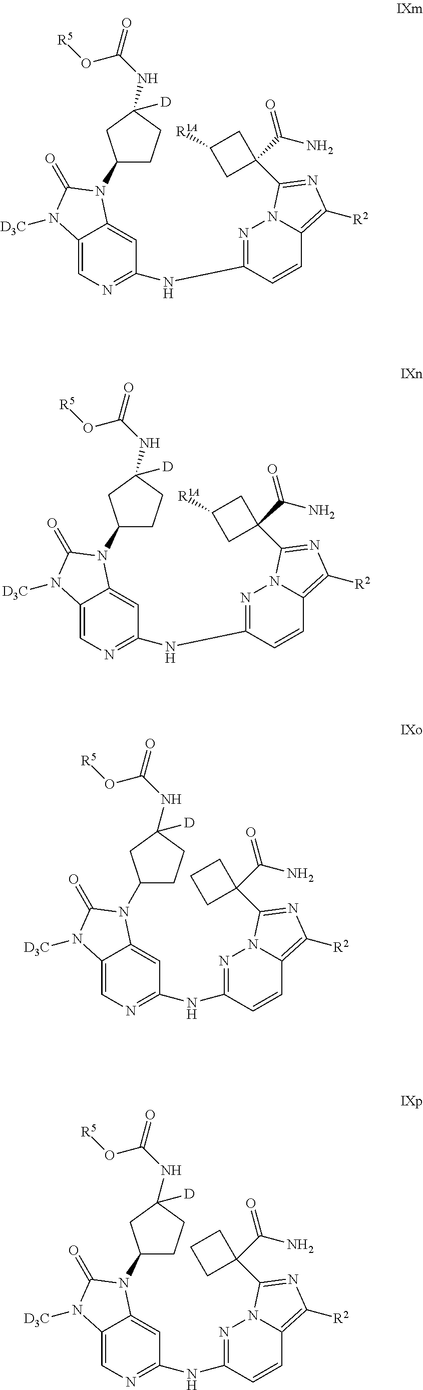

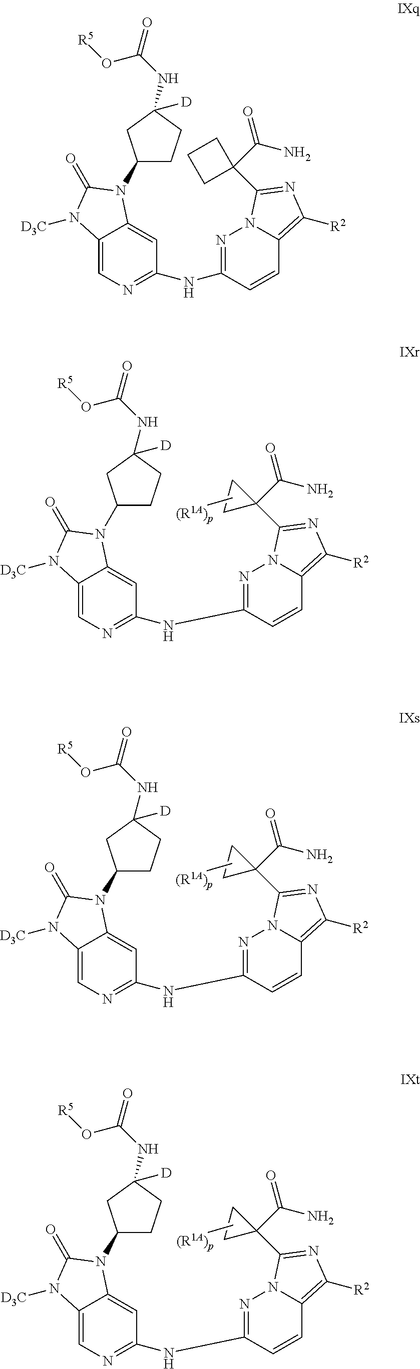

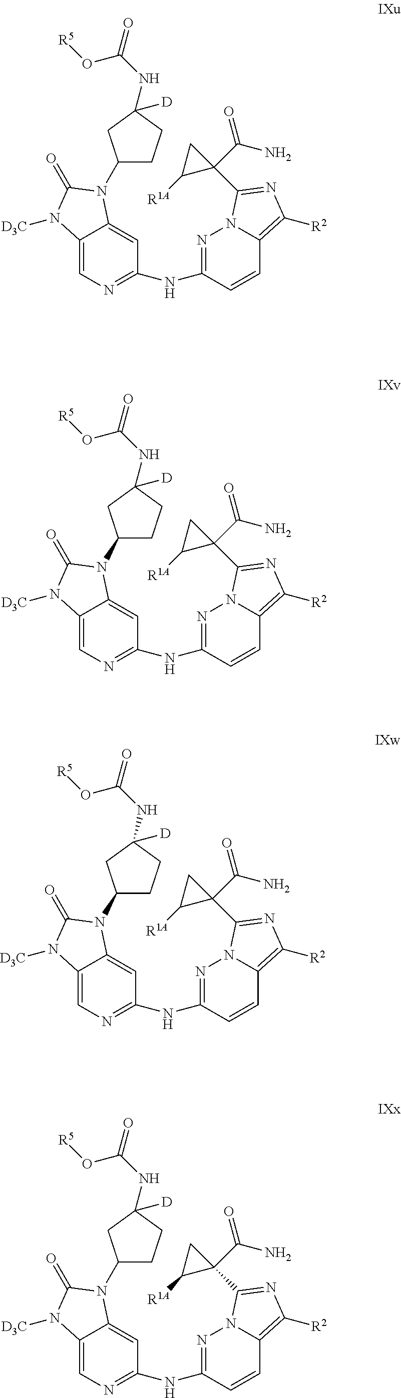

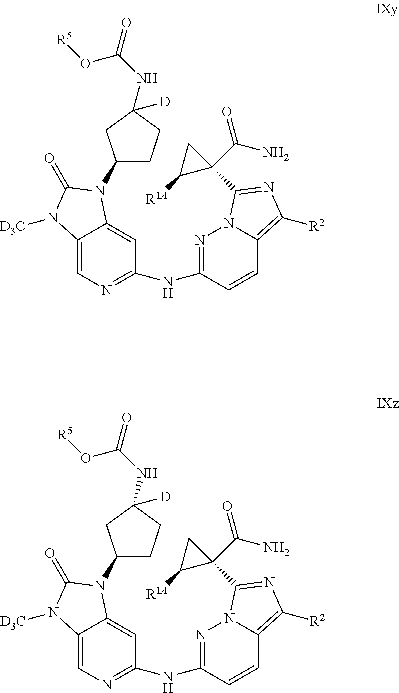

or a pharmaceutically acceptable salt thereof.

In some embodiments, the compound of Formula I or Formula Ia is a compound of Formula IXa, IXb, IXc, IXd, IXe, IXf, IXg, IXh, IXi, IXj, IXk, IXl, IXm, IXn, IXo, IXp, IXq, IXr, IXs, IXt, IXu, IXv, IXw, IXx, IXy, or IXz, wherein R⁵ is methyl.

In some embodiments, the compound of Formula I or Formula Ia is a compound of Formula IXa, IXb, IXc, IXd, IXe, IXf, IXg, IXh, IXi, IXj, IXk, IXl, IXm, IXn, IXo, IXp, IXq, IXr, IXs, IXt, IXu, IXv, IXw, IXx, IXy, or IXz, wherein R⁵ is trideuteromethyl.

In some embodiments, the compound of Formula I or Formula Ia is a compound of Formula X:

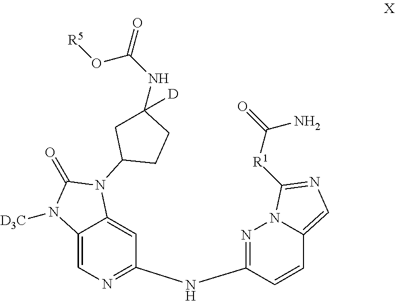

Or a pharmaceutically acceptable salt thereof. In some embodiments of Formula X, R⁵ is methyl. In some embodiments of Formula X, R⁵ is trideuteromethyl.

In some embodiments, the compound of Formula I or Formula Ia is a compound of Formula Xa, Xb, Xc, Xd, Xe, Xf, Xg, Xh, Xi, Xj, Xk, Xl, Xm, Xn, Xo, Xp, Xq, Xr, Xs, Xt, Xu, Xv, Xw, Xx, Xy, or Xz:

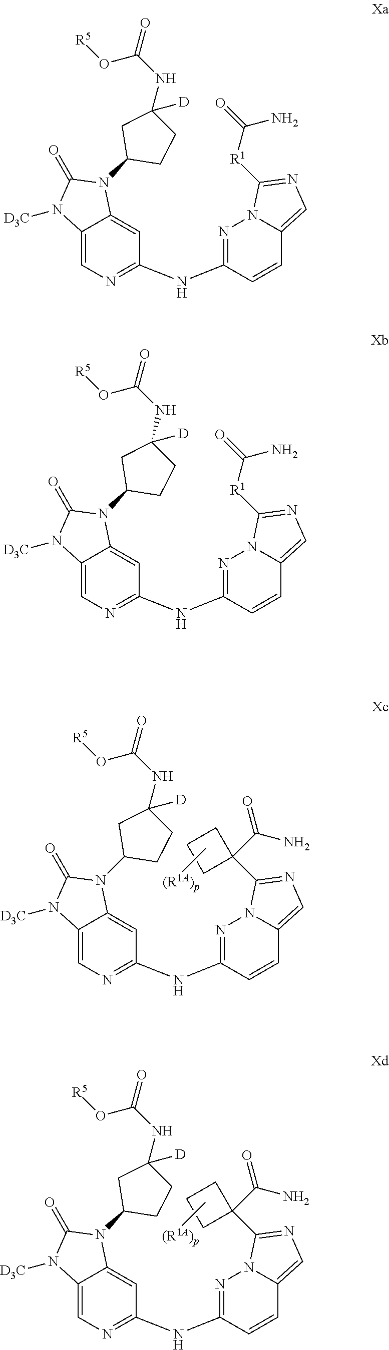

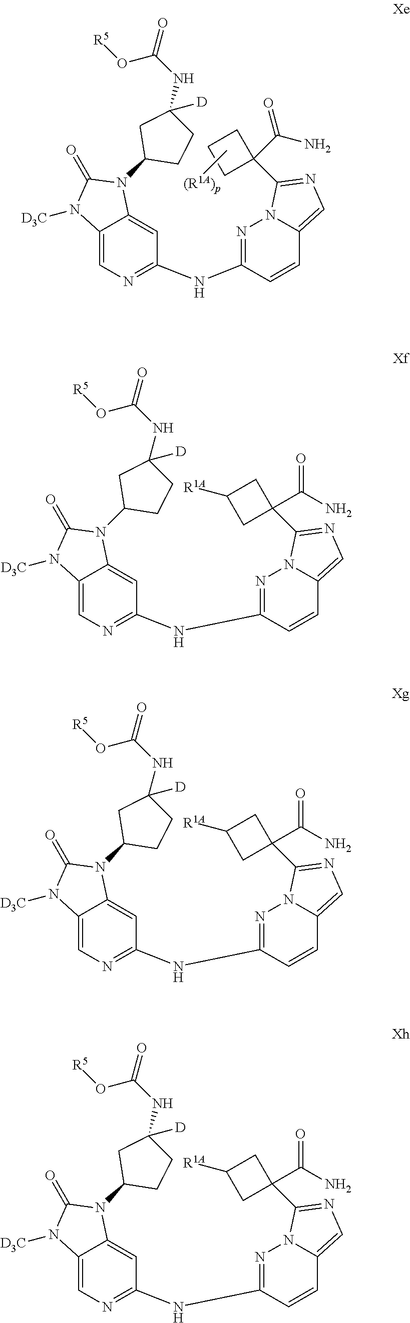

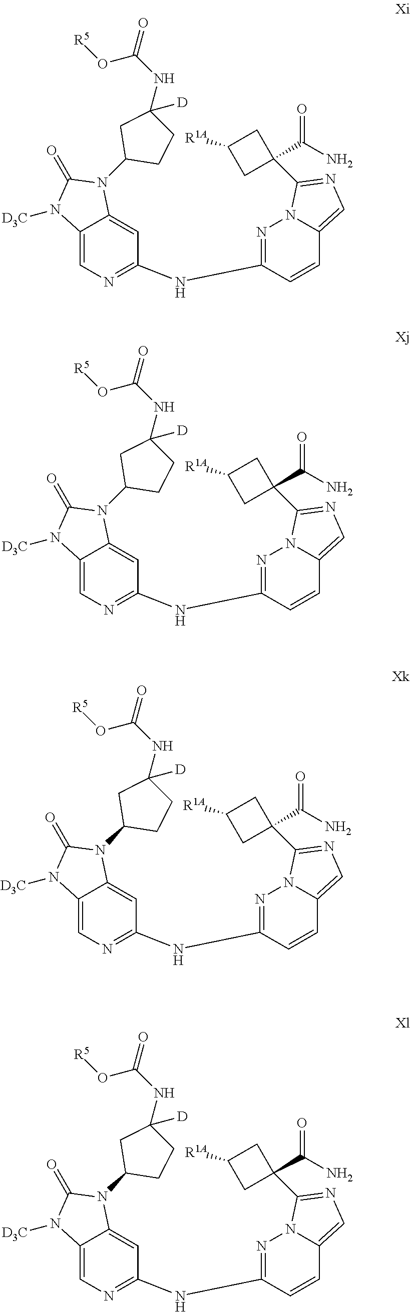

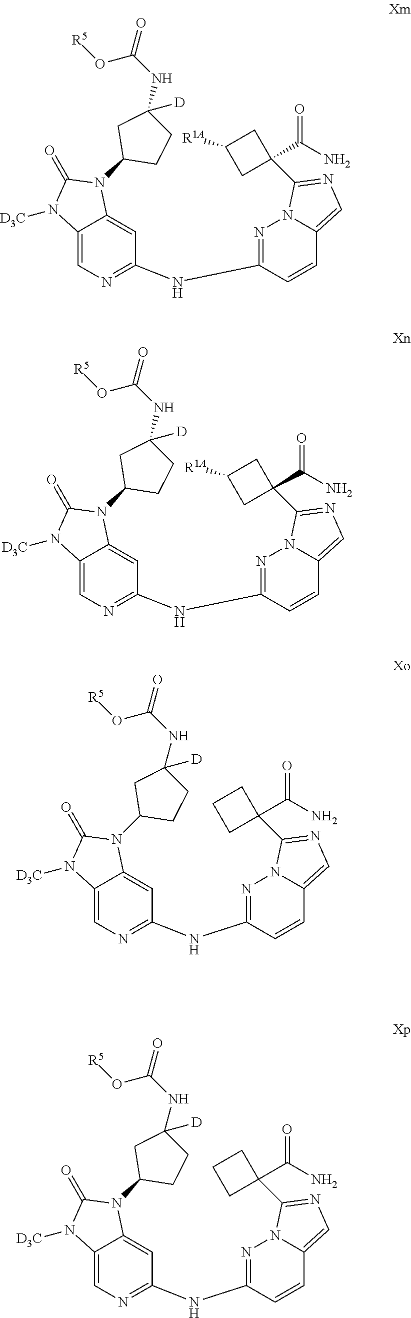

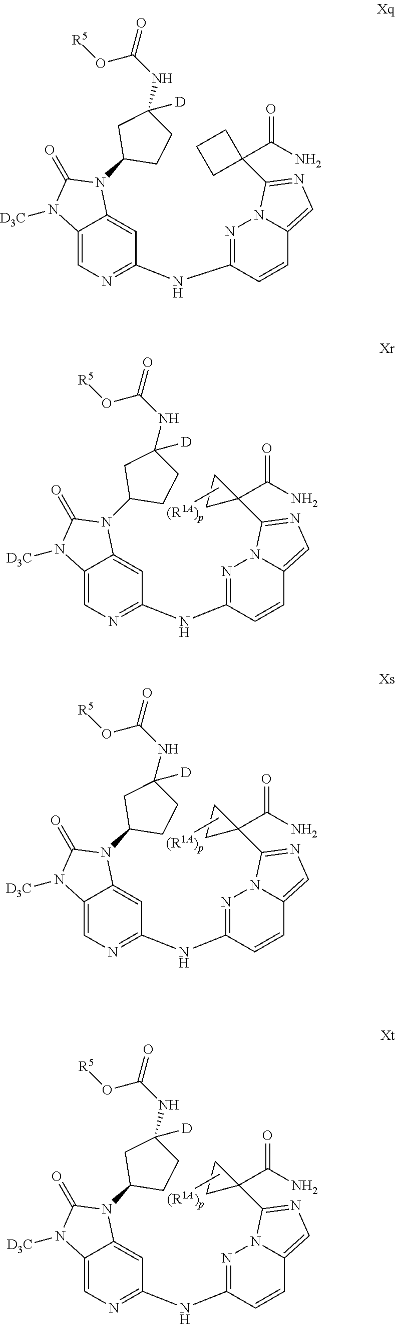

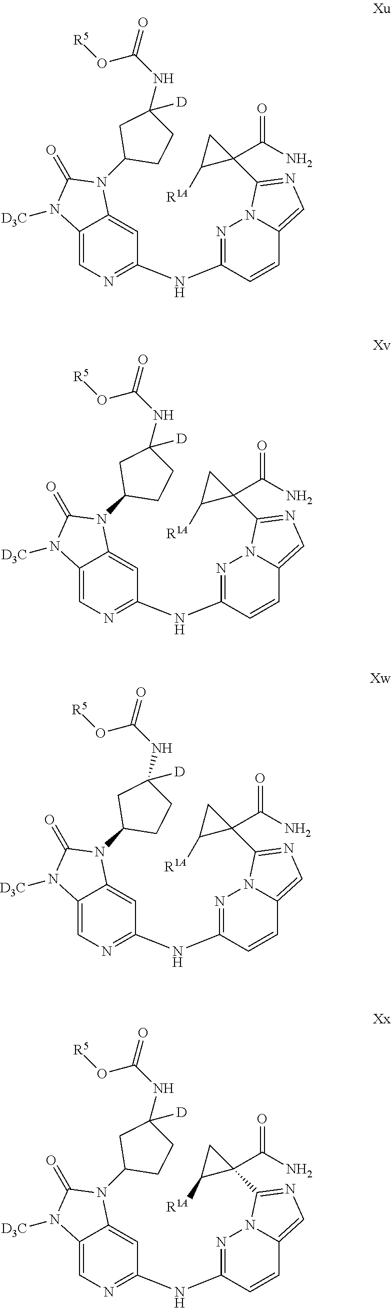

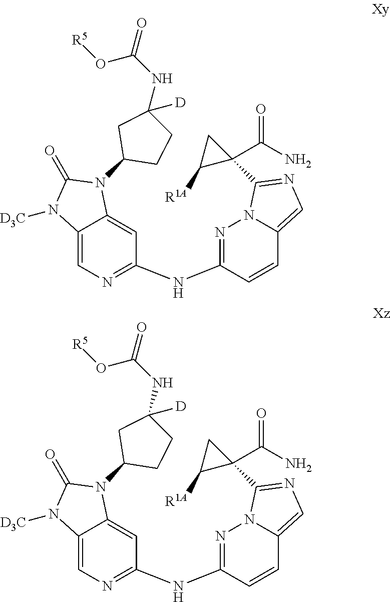

or a pharmaceutically acceptable salt thereof.

In some embodiments, the compound of Formula I or Formula Ia is a compound of Formula Xa, Xb, Xc, Xd, Xe, Xf, Xg, Xh, Xi, Xj, Xk, Xl, Xm, Xn, Xo, Xp, Xq, Xr, Xs, Xt, Xu, Xv, Xw, Xx, Xy, or Xz, wherein R⁵ is methyl.

In some embodiments, the compound of Formula I or Formula Ia is a compound of Formula Xa, Xb, Xc, Xd, Xe, Xf, Xg, Xh, Xi, Xj, Xk, Xl, Xm, Xn, Xo, Xp, Xq, Xr, Xs, Xt, Xu, Xv, Xw, Xx, Xy, or Xz, wherein R⁵ is trideuteromethyl.

In some embodiments, the compound of Formula I or Formula Ia is a compound of Formula XI:

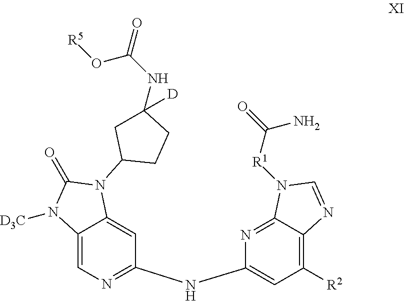

or a pharmaceutically acceptable salt thereof. In some embodiments of Formula XI, R⁵ is methyl. In some embodiments of Formula XI, R⁵ is trideuteromethyl.

In some embodiments, the compound of Formula I or Formula Ia is a compound of Formula XIa, XIb, XIc, XId, XIe, XIf, XIg, XIh, XIi, XIj, XIk, XIl, XIm, XIn, XIo, XIp, or XIq:

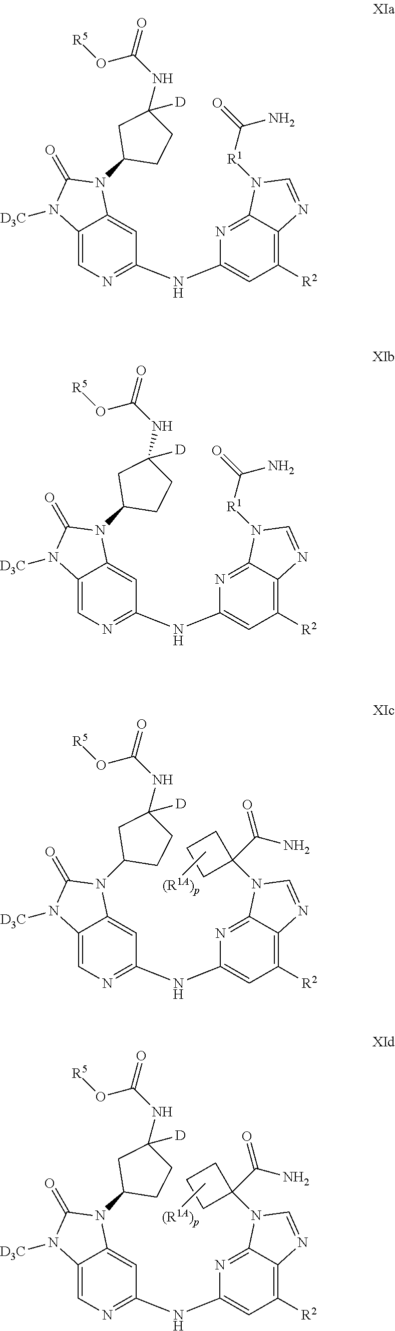

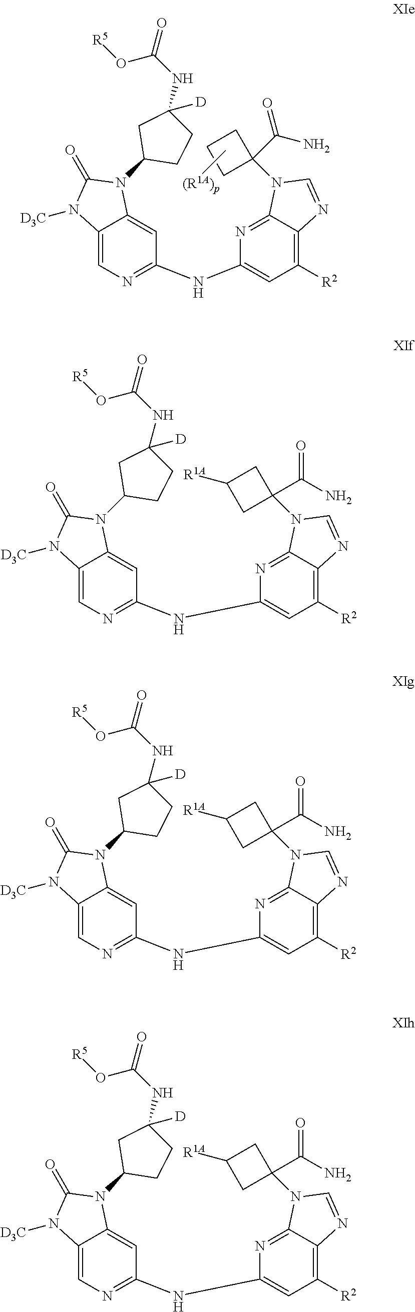

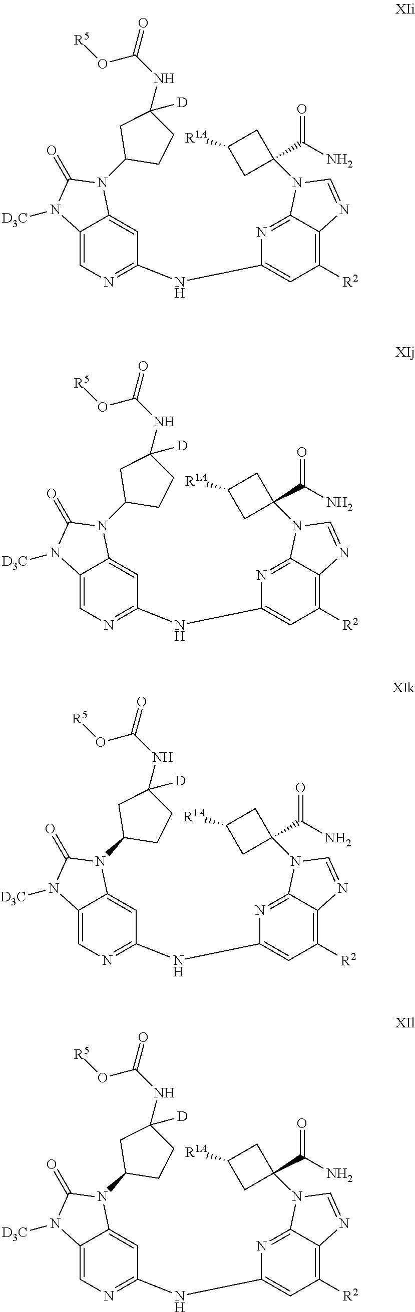

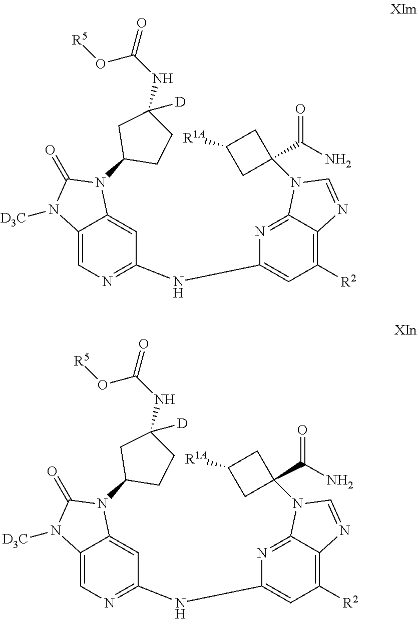

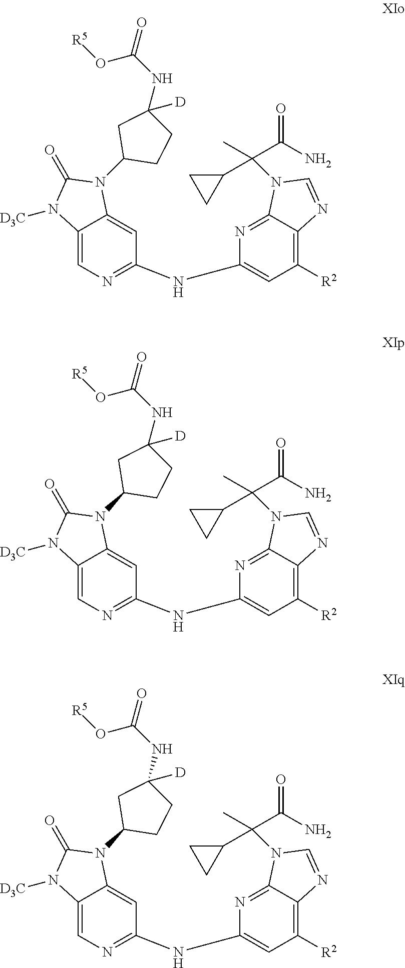

or a pharmaceutically acceptable salt thereof.

In some embodiments, the compound of Formula I or Formula Ia is a compound of Formula XIa, XIb, XIc, XId, XIe, XIf, XIg, XIh, XIi, XIj, XIk, XII, XIm, XIn, XIo, XIp, or XIq, wherein R⁵ is methyl.

In some embodiments, the compound of Formula I or Formula Ia is a compound of Formula XIa, XIb, XIc, XId, XIe, XIf, XIg, XIh, XIi, XIj, XIk, XII, XIm, XIn, XIo, XIp, or XIq, wherein R⁵ is trideuteromethyl.

In some embodiments, the compound of Formula I or Formula Ia is a compound of Formula XII:

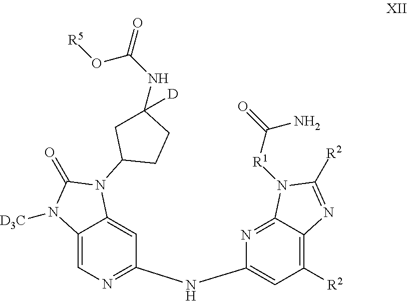

or a pharmaceutically acceptable salt thereof. In some embodiments of Formula XII, R⁵ is methyl. In some embodiments of Formula XII, R⁵ is trideuteromethyl.

In some embodiments, the compound of Formula I or Formula Ia is a compound of Formula XIIa, XIIb, XIIc, XIId, XIIe, XIIf, XIIg, XIIh, XIIi, XIIj, XIIk, XIIl, XIIm, XIIn, XIIo, XIIp, or XIIq:

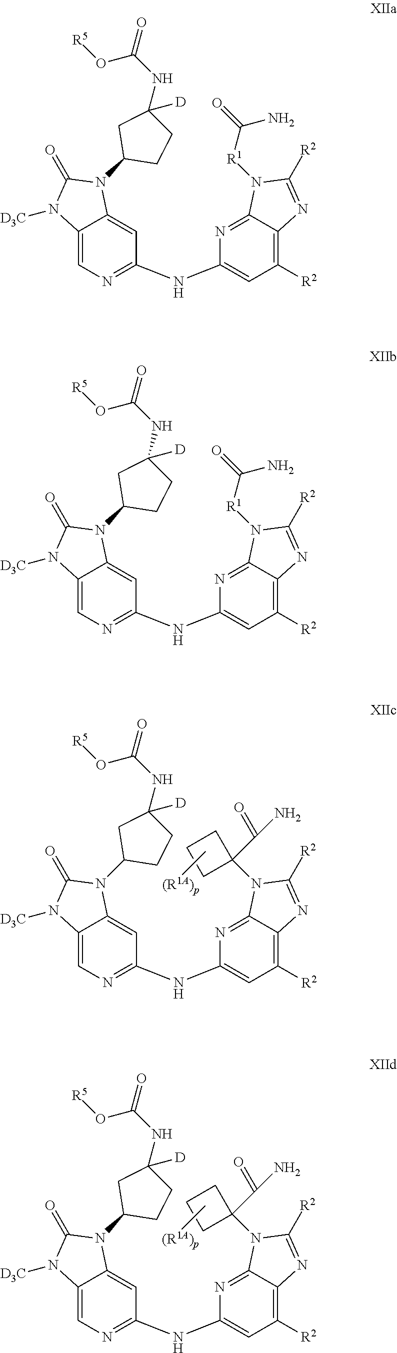

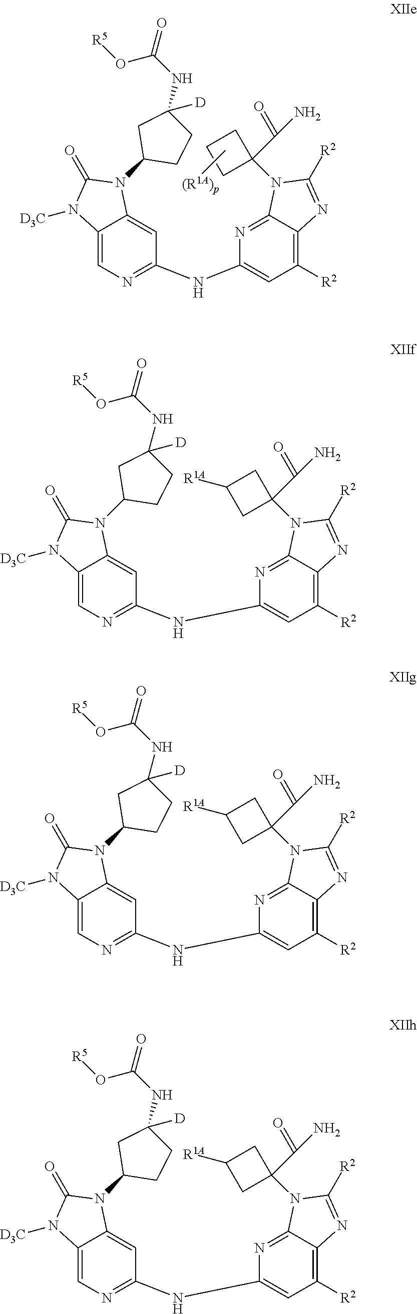

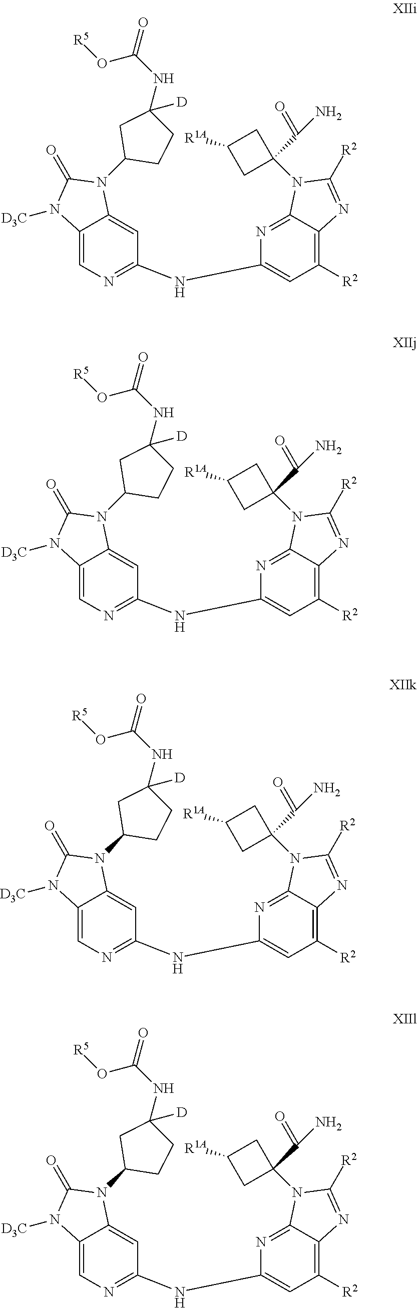

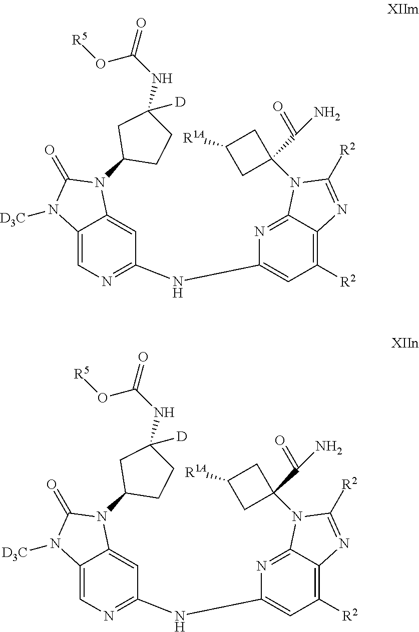

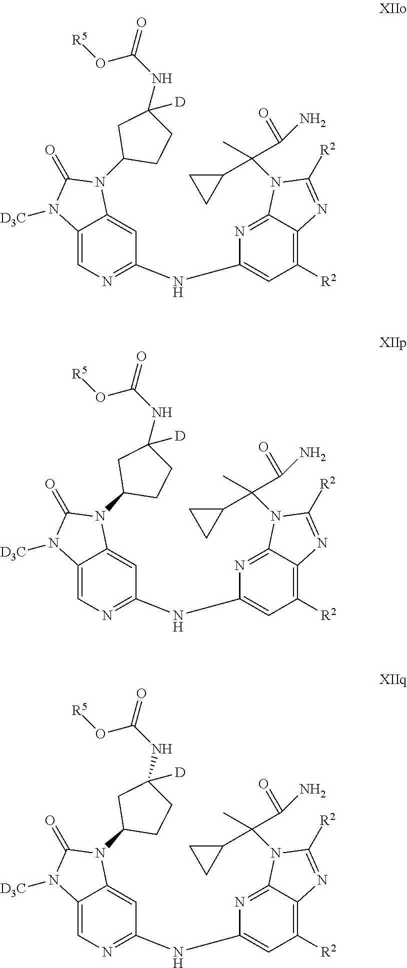

or a pharmaceutically acceptable salt thereof.

In some embodiments, the compound of Formula I or Formula Ia is a compound of Formula XIIa, XIIb, XIIc, XIId, XIIe, XIIf, XIIg, XIIh, XIIi, XIIj, XIIk, XIIl, XIIm, XIIn, XIIo, XIIp, or XIIq, wherein R⁵ is methyl.

In some embodiments, the compound of Formula I or Formula Ia is a compound of Formula XIIa, XIIb, XIIc, XIId, XIIe, XIIf, XIIg, XIIh, XIIi, XIIj, XIIk, XIIl, XIIm, XIIn, XIIo, XIIp, or XIIq, wherein R⁵ is trideuteromethyl.

In some embodiments, the compound of Formula I or Formula Ia is a compound of Formula XIII:

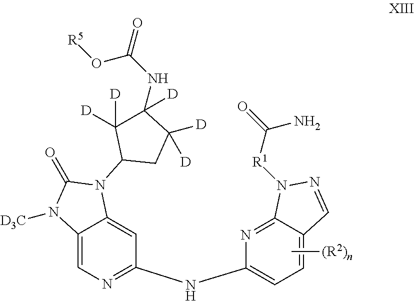

or a pharmaceutically acceptable salt thereof. In some embodiments of Formula XIII, R⁵ is methyl. In some embodiments of Formula XIII, R⁵ is trideuteromethyl.

In some embodiments, the compound of Formula I or Formula Ia is a compound of Formula XIIIa, XIIIb, XIIIc, XIIId, XIIIe, XIIIf, XIIIg, or XIIIh:

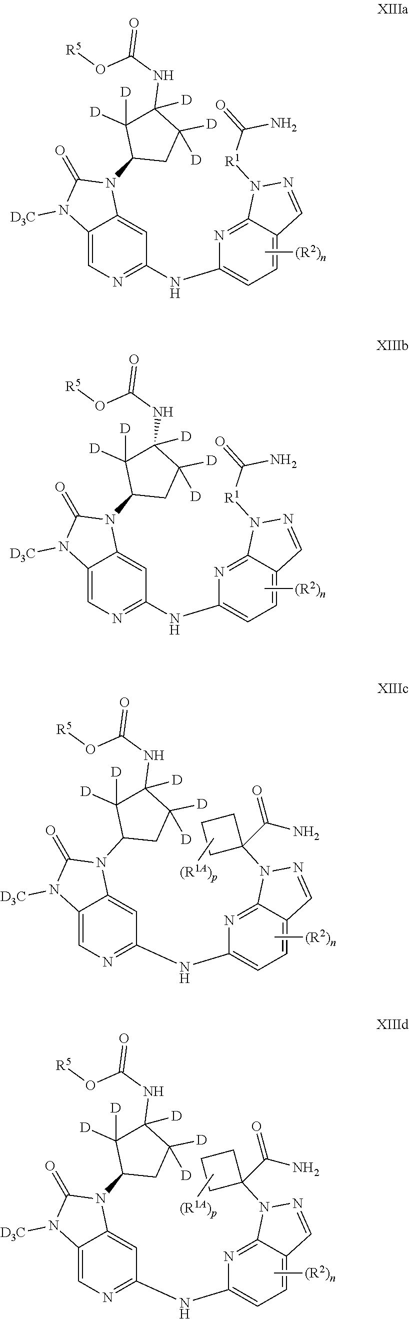

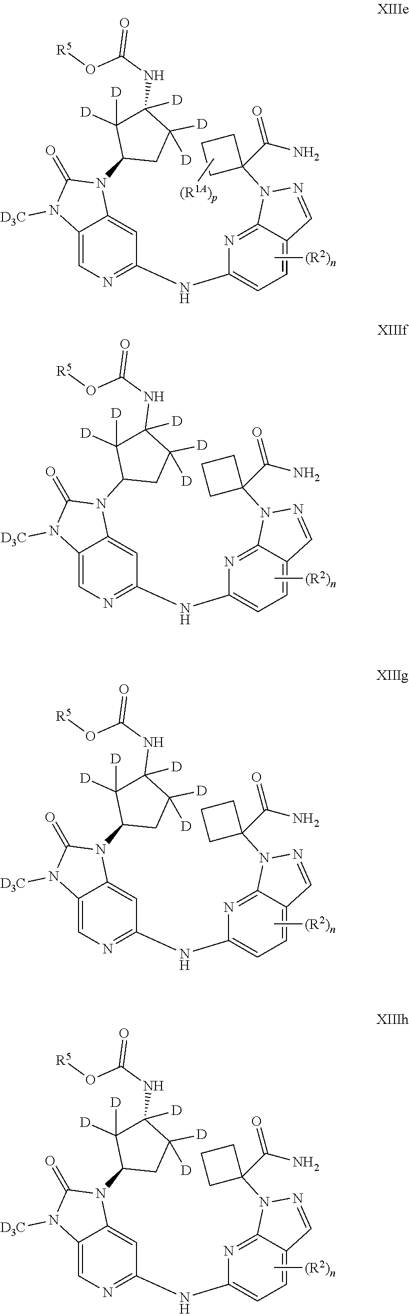

or a pharmaceutically acceptable salt thereof.

In some embodiments, the compound of Formula I or Formula Ia is a compound of Formula XIIIa, XIIIb, XIIIc, XIIId, XIIIe, XIIIf, XIIIg, or XIIIh, wherein R⁵ is methyl.

In some embodiments, the compound of Formula I or Formula Ia is a compound of Formula XIIIa, XIIIb, XIIIc, XIIId, XIIIe, XIIIf, XIIIg, or XIIIh, wherein R⁵ is trideuteromethyl.

In some embodiments, the compound of Formula I or Formula Ia is a compound of Formula XIV:

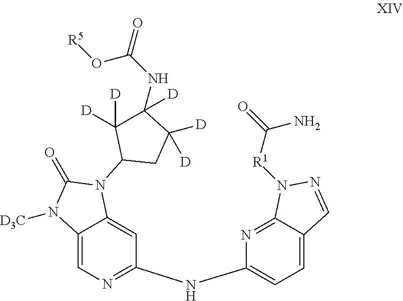

or a pharmaceutically acceptable salt thereof. In some embodiments of Formula XIV, R⁵ is methyl. In some embodiments of Formula XIV, R⁵ is trideuteromethyl.

In some embodiments, the compound of Formula I or Formula Ia is a compound of Formula XIVa, XIVb, XIVc, XIVd, XIVe, XIVf, XIVg, or XIVh:

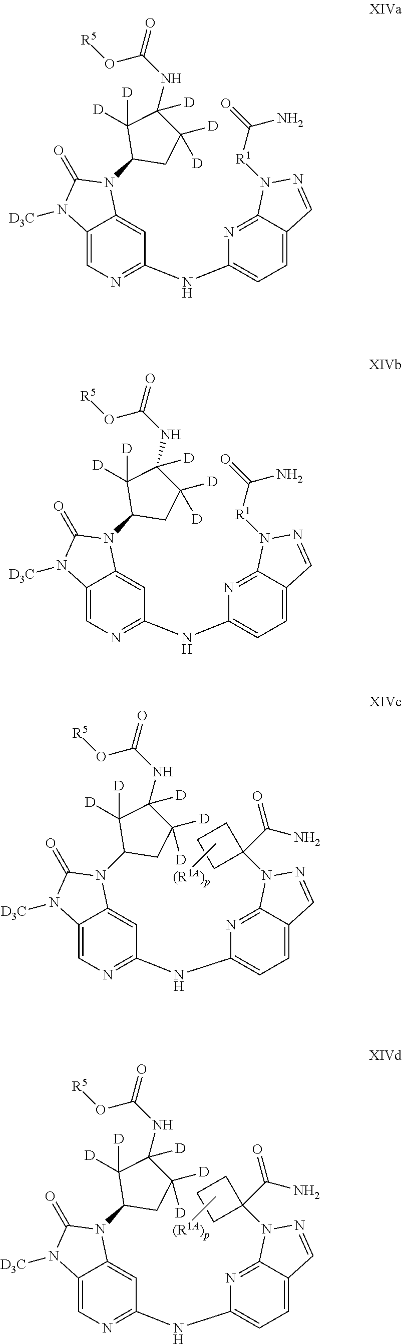

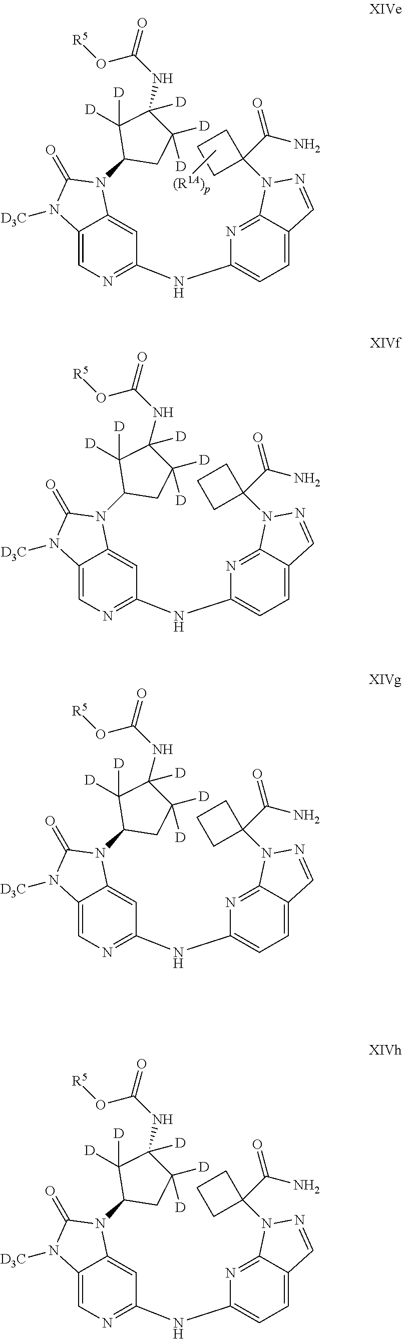

or a pharmaceutically acceptable salt thereof.

In some embodiments, the compound of Formula I or Formula Ia is a compound of Formula XIVa, XIVb, XIVc, XIVd, XIVe, XIVf, XIVg, or XIVh, wherein R⁵ is methyl.

In some embodiments, the compound of Formula I or Formula Ia is a compound of Formula XIVa, XIVb, XIVc, XIVd, XIVe, XIVf, XIVg, or XIVh, wherein R⁵ is trideuteromethyl.

In some embodiments, the compound of Formula I or Formula Ia is a compound of Formula XV:

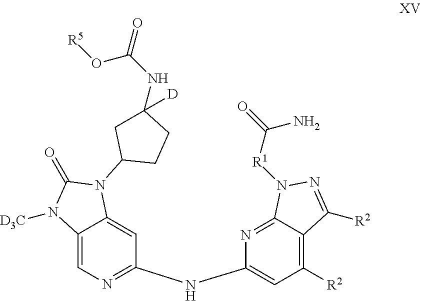

or a pharmaceutically acceptable salt thereof. In some embodiments of Formula XV, R⁵ is methyl. In some embodiments of Formula XV, R⁵ is trideuteromethyl.

In some embodiments, the compound of Formula I or Formula Ia is a compound of Formula XVa, XVb, XVc, XVd, XVe, XVf, XVg, XVh, XVi, XVj, XVk, XVm, XVn, XVo, or XVp:

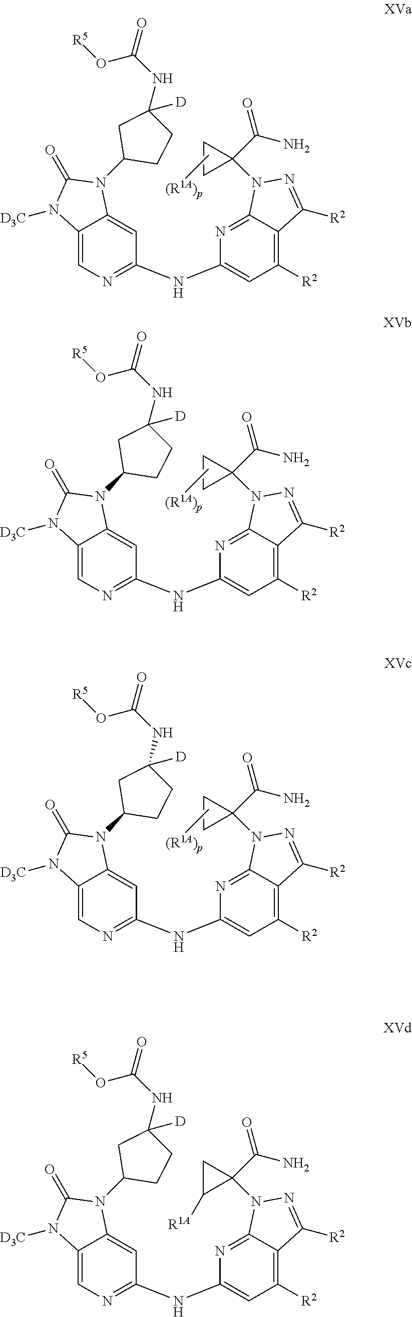

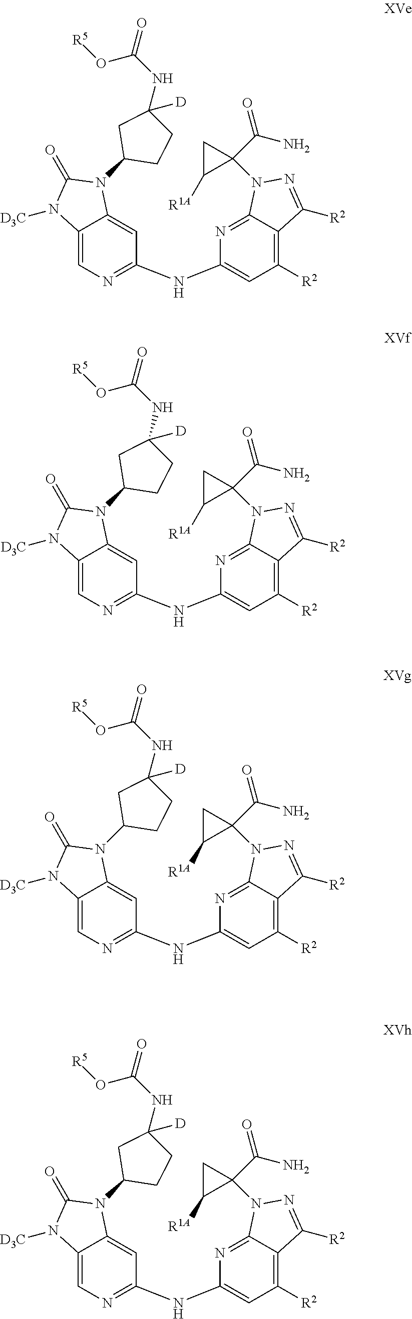

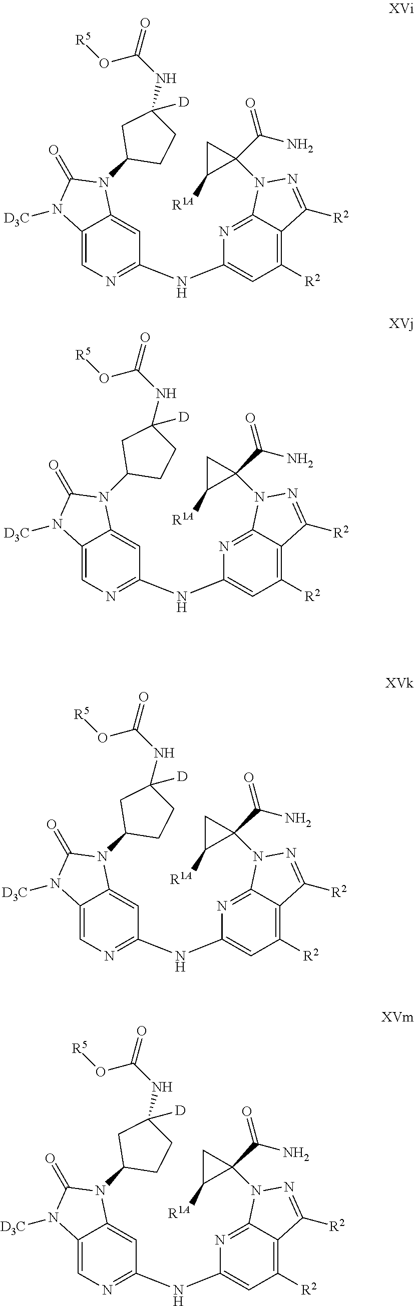

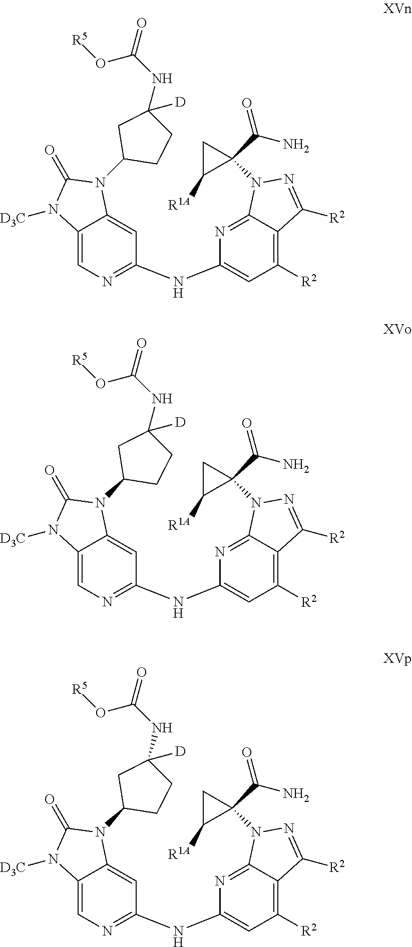

or a pharmaceutically acceptable salt thereof.

In some embodiments, the compound of Formula I or Formula Ia is a compound of Formula XVa, XVb, XVc, XVd, XVe, XVf, XVg, XVh, XVi, XVj, XVk, XVm, XVn, XVo, or XVp, wherein R⁵ is methyl.

In some embodiments, the compound of Formula I or Formula Ia is a compound of Formula XVa, XVb, XVc, XVd, XVe, XVf, XVg, XVh, XVi, XVj, XVk, XVm, XVn, XVo, or XVp, wherein R⁵ is trideuteromethyl.

In some embodiments, the compound of Formula Ia is a compound of Formula XVI:

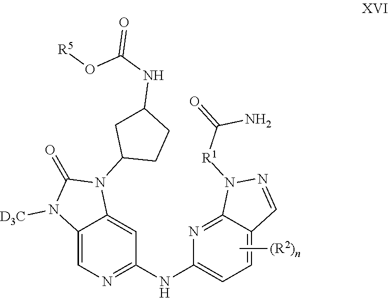

or a pharmaceutically acceptable salt thereof. In some embodiments of Formula XVI, R⁵ is methyl. In some embodiments of Formula XVI, R⁵ is trideuteromethyl.

In some embodiments, the compound of Formula Ia is a compound of Formula XVIa, XVIb, XVIc, XVId, XVIe, XVIf, XVIg, XVIh, XVIi, XVIj, XVIk, XVIm, XVIn, XVIo, XVIp, XVIq, XVIr, XVIs, XVIt, XVIu, XVIv, XVIw, XVIx, XVIy, XVIz, XVIaa, or XVIab:

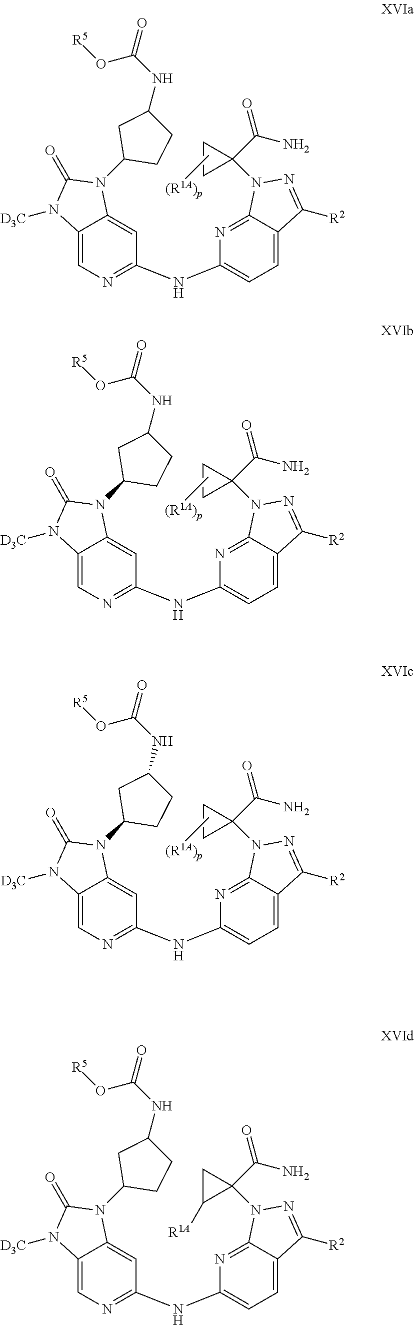

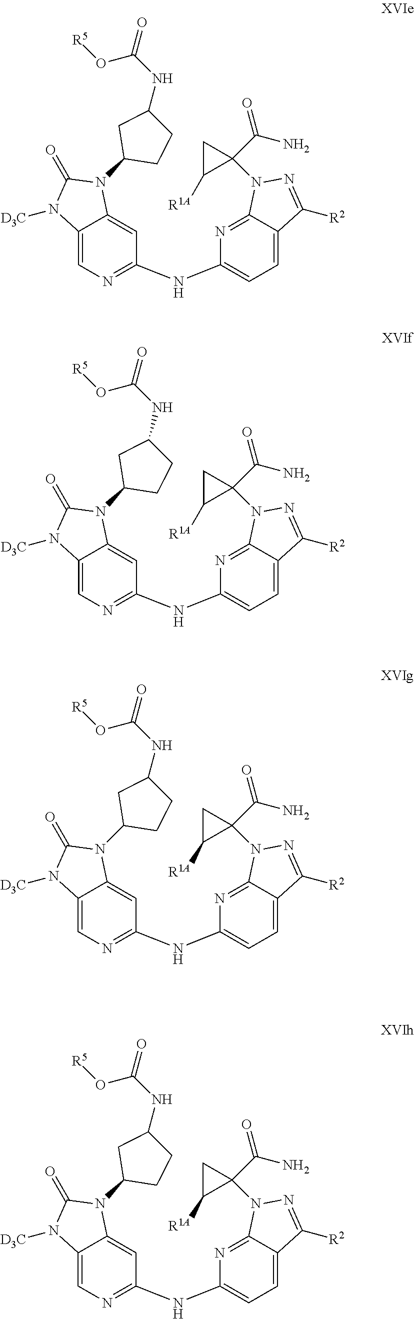

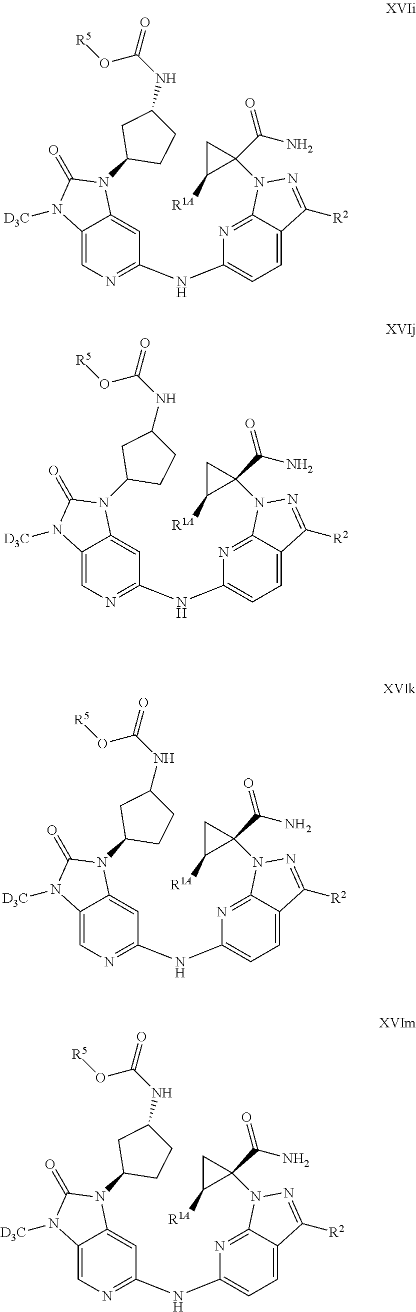

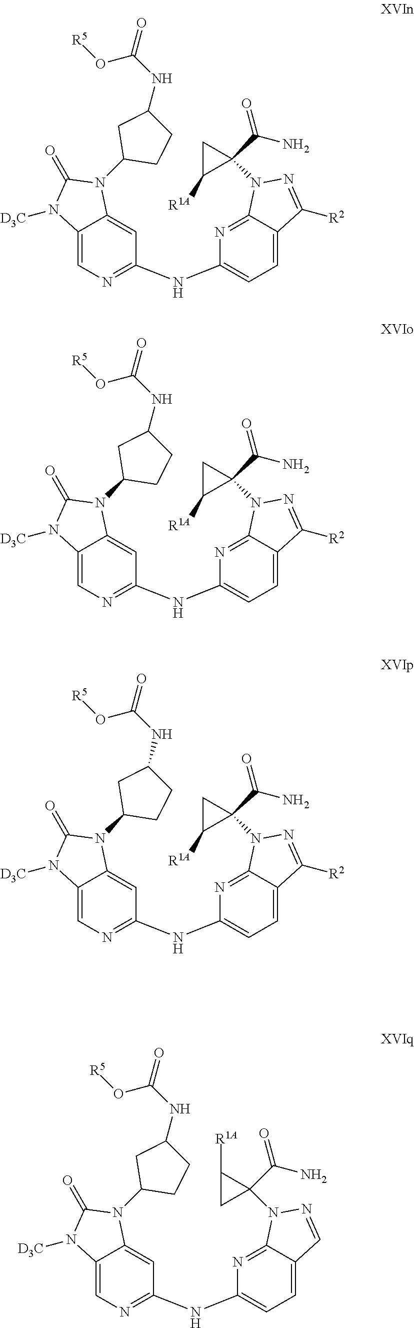

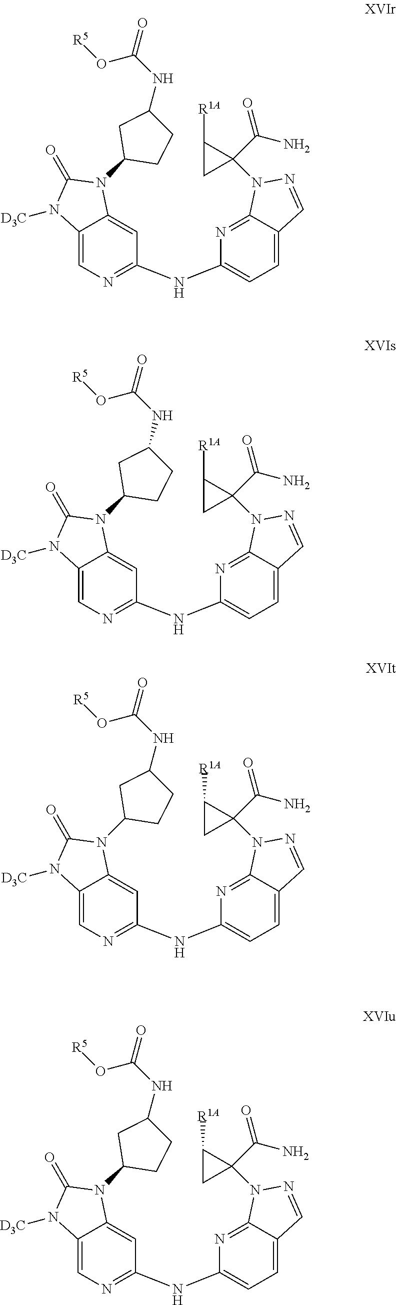

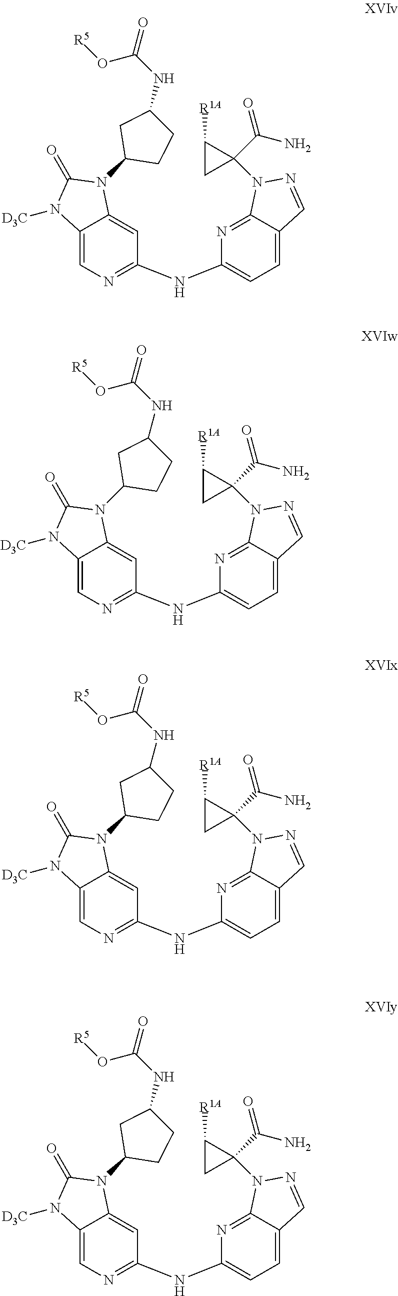

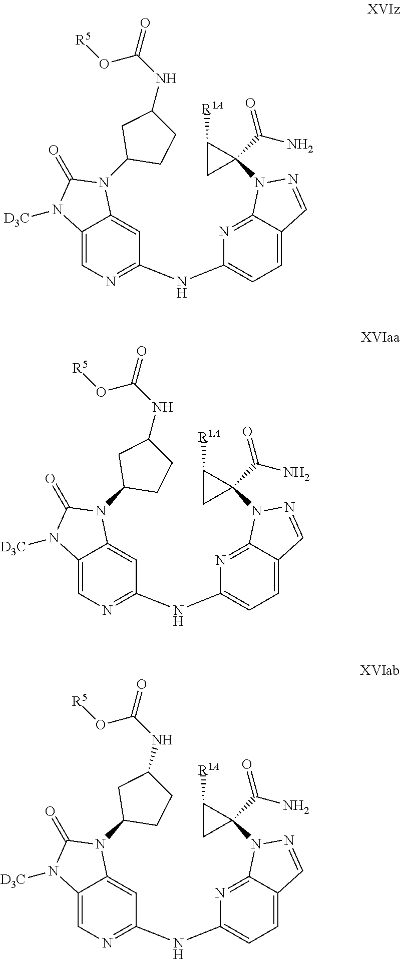

or a pharmaceutically acceptable salt thereof.

In some embodiments, the compound of Formula Ia is a compound of Formula XVIa, XVIb, XVIc, XVId, XVIe, XVIf, XVIg, XVIh, XVIi, XVIj, XVIk, XVIm, XVIn, XVIo, XVIp, XVIq, XVIr, XVIs, XVIt, XVIu, XVIv, XVIw, XVIx, XVIy, XVIz, XVIaa, or XVIab, wherein R⁵ is methyl.

In some embodiments, the compound of Formula Ia is a compound of Formula XVIa, XVIb, XVIc, XVId, XVIe, XVIf, XVIg, XVIh, XVIi, XVIj, XVIk, XVIm, XVIn, XVIo, XVIp, XVIq, XVIr, XVIs, XVIt, XVIu, XVIv, XVIw, XVIx, XVIy, XVIz, XVIaa, or XVIab, wherein R⁵ is trideuteromethyl.

In some embodiments, the compound provided herein is selected from:
methyl (3-(6-((1-(1-carbamoylcyclopropyl)-4-(tetrahydro-2H-pyran-4-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoylcyclobutyl)-4-cyclopropyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoylcyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-2-methylcyclopropyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-3-fluorocyclobutyl)-5-cyclopropylimidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-3-fluorocyclobutyl)-5-(tetrahydro-2H-pyran-4-yl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-3-fluorocyclobutyl)-5-cyclobutylimidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-3-fluorocyclobutyl)imidazo[1,5-b]pyridazin-2-yl-5-d)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-3-fluorocyclobutyl)-5-(2-hydroxypropan-2-yl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoylcyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1,2,2,5,5-d5)carbamate;
methyl (3-(6-((1-(1-carbamoylcyclobutyl)-4-(4-hydroxycyclohexyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-3,3-difluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-3-methoxycyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoylcyclobutyl)-3-(1-hydroxyethyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoylcyclobutyl)-3-(2-hydroxyethyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoylcyclobutyl)-3-(2-hydroxypropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-3-fluorocyclobutyl)-3-(2-hydroxypropan-2-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((3-(1-carbamoyl-3-fluorocyclobutyl)-7-methyl-2-(trifluoromethyl)-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl (3-(6-((3-(1-carbamoyl-3-fluorocyclobutyl)-2-(difluoromethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl (3-(6-((3-(1-amino-2-cyclopropyl-1-oxopropan-2-yl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-ethyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(2-methoxypyridin-3-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(4-methoxypyridin-3-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-isopropylcyclopropyl)-3-((methoxycarbonyl)amino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-(trifluoromethyl)cyclopropyl)-3-((methoxycarbonyl)amino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-isopropylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(dimethylcarbamoyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-morpholino-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(3-methyl-2-oxoimidazolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(3,3-difluoroazetidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(2-oxopyrrolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-ethynylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-(trifluoromethyl)cyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-(trifluoromethyl)cyclopropyl)-3-(methyl-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-(trifluoromethyl)cyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-(difluoromethyl)cyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-ethylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-ethylcyclopropyl)-3-(3,3-difluoroazetidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(2-carbamoyl-[1,1′-bi(cyclopropan)]-2-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-ethylcyclopropyl)-3-(methyl-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(2-carbamoyl-[1,1′-bi(cyclopropan)]-2-yl)-3-(methyl-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(2-carbamoyl-[1,1′-bi(cyclopropan)]-2-yl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(2-carbamoyl-[1,1′-bi(cyclopropan)]-2-yl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-4-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((3R)-3-(6-((1-(1-carbamoyl-2-methylcyclopropyl)-3-(2,2,2-trifluoroethyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(methyl-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3,4-dimethyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(methyl-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl (3-(6-((1-(1-carbamoyl-2-(difluoromethyl)cyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-2-methylcyclopropyl)-5-(methyl-d3)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-2-methylcyclopropyl)-5-(trifluoromethyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)-4-fluorocyclopentyl)carbamate;
methyl (3-(6-((1-(1-carbamoyl-2-ethylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)-4-fluorocyclopentyl)carbamate;
methyl (3-(6-((1-(1-carbamoyl-2-methylcyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)-4-fluorocyclopentyl)carbamate;
methyl (3-(6-((1-(1-carbamoyl-2-methylcyclopropyl)-3-(2-hydroxypropan-2-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl-d3 (3-(6-((1-(1-carbamoyl-2-methylcyclopropyl)-3-(tetrahydro-2H-pyran-4-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(tetrahydro-2H-pyran-4-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl-d3 (3-(6-((1-(1-carbamoyl-2-methylcyclopropyl)-3-(dimethylcarbamoyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 (3-(6-((1-(1-carbamoyl-2-methylcyclopropyl)-3-(dimethylamino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((3-(bis(methyl-d3)amino)-1-(1-carbamoyl-2-methylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(4-fluoro-2-azabicyclo[2.1.1]hexan-2-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (1-(1-carbamoyl-3-fluorocyclobutyl)-6-((1-(3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl (1-(1-carbamoyl-2-methylcyclopropyl)-6-((1-(3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl (1-(1-carbamoyl-2-methylcyclopropyl)-6-((1-(3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl (1-((1S)-1-carbamoyl-2-methylcyclopropyl)-6-((1-(3-(((methoxy-d3)carbonyl)amino)cyclopentyl)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl (3-(6-((1-(1-carbamoyl-2-methylcyclopropyl)-3-((methoxycarbonyl)amino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(2-carbamoyl-[1,1′-bi(cyclopropan)]-2-yl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl-d3 (3-(6-((3-(1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl)carbamate;
methyl-d3 (3-(6-((3-(1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl (3-(6-((3-(1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 (3-(6-((3-(1-carbamoyl-2-methylcyclopropyl)-2-(methoxymethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl (3-(6-((3-(1-carbamoyl-2-methylcyclopropyl)-2-(methoxymethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl (3-(6-((3-(1-carbamoyl-2-methylcyclopropyl)-2-(1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 (3-(6-((3-(1-carbamoyl-2-methylcyclopropyl)-2-(1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl)carbamate;
methyl-d3 (3-(6-((3-(1-carbamoyl-2-methylcyclopropyl)-2-(1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl (3-(6-((3-(1-carbamoyl-2-methylcyclopropyl)-2-(1-methoxyethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 (3-(6-((1-(1-carbamoyl-2-methylcyclopropyl)-3-(methoxy-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 (3-(6-((1-(1-carbamoyl-2-methylcyclopropyl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 (3-(6-((1-(1-carbamoyl-2-methylcyclopropyl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(methoxy-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl-d3 (3-(6-((1-(1-carbamoyl-2-methylcyclopropyl)-3-(methoxy-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl-d3 (3-(6-((1-(1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 (3-(6-((1-(1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 (3-(6-((1-(1-carbamoyl-2-methylcyclopropyl)-3-(1-cyanocyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 (3-(6-((1-(1-carbamoyl-2-methylcyclopropyl)-3-(1-cyanocyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-methylcyclopropyl)-3-(1-cyanocyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((1-(1-carbamoyl-2-(difluoromethyl)cyclopropyl)-3-(methoxy-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-2-methylcyclopropyl)-4-methyl-5-(methyl-d3)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl (3-(6-((7-(1-carbamoyl-2-methylcyclopropyl)-4,5-dimethylimidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate;
methyl-d3 (4-(6-((1-(1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)-2-fluorocyclopentyl)carbamate;
methyl (4-(6-((1-(1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)-2-fluorocyclopentyl)carbamate;
methyl-d3 (3-(6-((1-(2-carbamoyl-[1,1′-bi(cyclopropan)]-2-yl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)-4-fluorocyclopentyl)carbamate;
methyl-d3 (3-(6-((1-(2-carbamoyl-[1,1′-bi(cyclopropan)]-2-yl)-3-(methoxy-d3)-1H-pyrazolo[3,4-b]pyridin-6-yl)amino)-3-(methyl-d3)-2-oxo-2,3-dihydro-1H-imidazo[4,5-c]pyridin-1-yl)-4-fluorocyclopentyl)carbamate;
methyl (3-(6-((1-(2-carbamoyl-[1,1′-bi(cyclopropan)]-2-yl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)-4-fluorocyclopentyl)carbamate;
methyl (4-(6-((1-(1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)-2-methoxycyclopentyl)carbamate;
methyl (1-(1-carbamoyl-2-ethynylcyclopropyl)-6-((1-(3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate; and
methyl (3-(6-((1-(1-carbamoyl-2-ethynylcyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
   or a pharmaceutically acceptable salt thereof.

In some embodiments, the compound provided herein is selected from:
methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1,2,2,5,5-d₅)carbamate;
methyl ((1R,3R)-3-(6-((1-(1-carbamoylcyclobutyl)-4-(trans-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-methoxycyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-7-methyl-2-(trifluoromethyl)-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-ethyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(2-methoxypyridin-3-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(4-methoxypyridin-3-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2R)-1-carbamoyl-2-isopropylcyclopropyl)-3-((methoxycarbonyl)amino)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(trifluoromethyl)cyclopropyl)-3-((methoxycarbonyl)amino)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2R)-1-carbamoyl-2-isopropylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-morpholino-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(3-methyl-2-oxoimidazolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(3,3-difluoroazetidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(2-oxopyrrolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((1-((1S,2R)-1-carbamoyl-2-ethynylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(trifluoromethyl)cyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(trifluoromethyl)cyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(trifluoromethyl)cyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(difluoromethyl)cyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-ethylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-ethylcyclopropyl)-3-(3,3-difluoroazetidin-1-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1R,2S)-2-carbamoyl-[1,1′-bi(cyclopropan)]-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-ethylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1R,2S)-2-carbamoyl-[1,1′-bi(cyclopropan)]-2-yl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-4-(1-methyl-1H-pyrazol-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-4-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(2,2,2-trifluoroethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3,4-dimethyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((1-((1S,2S)-1-carbamoyl-2-(difluoromethyl)cyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1R,2S)-1-carbamoyl-2-methylcyclopropyl)-5-(methyl-d₃)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1R,2S)-1-carbamoyl-2-methylcyclopropyl)-5-(trifluoromethyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl (3-(6-((1-((1S,2S)-1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate;
methyl (3-(6-((1-((1S,2S)-1-carbamoyl-2-ethylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate;
methyl (3-(6-((1-((1S,2S)-1-carbamoyl-2-methylcyclopropyl)-3-methyl-1H-pyrazolo[3,4-b]pyridin-6-yl)amino)-3-(methyl-d₃)-2-oxo-2,3-dihydro-H-imidazo[4,5-c]pyridin-1-yl)-4-fluorocyclopentyl)carbamate;
methyl ((1R,3R)-3-(6-((1-((1S,2S)-1-carbamoyl-2-methylcyclopropyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl-d₃ ((1R,3R)-3-(6-((1-((1S,2S)-1-carbamoyl-2-methylcyclopropyl)-3-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl-d3 ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 ((1R,3R)-3-(6-((1-((1S,2S)-1-carbamoyl-2-methylcyclopropyl)-3-(dimethylamino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-(bis(methyl-d3)amino)-1-((1S,2S)-1-carbamoyl-2-methylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(4-fluoro-2-azabicyclo[2.1.1]hexan-2-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (1-((1S,3S)-1-carbamoyl-3-fluorocyclobutyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
ethyl (1-((1S,2S)-1-carbamoyl-2-methylcyclopropyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl (1-((1S,2S)-1-carbamoyl-2-methylcyclopropyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl (1-((1S,2S)-1-carbamoyl-2-methylcyclopropyl)-6-((1-((1R,3R)-3-(((methoxy-d3)carbonyl)amino)cyclopentyl)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl ((1R,3R)-3-(6-((1-((1S,2S)-1-carbamoyl-2-methylcyclopropyl)-3-((methoxycarbonyl)amino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (1-((1S,2R)-1-carbamoyl-2-ethynylcyclopropyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl ((1R,3R)-3-(6-((1-((1S,2R)-1-carbamoyl-2-ethynylcyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((1-((1R,2S)-2-carbamoyl-[1,1′-bi(cyclopropan)]-2-yl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl-d3 ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl)carbamate;
methyl-d3 ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 ((1R,3R)-3-(6-((3-((1S,2S)-1-carbamoyl-2-methylcyclopropyl)-2-(methoxymethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-((1S,2S)-1-carbamoyl-2-methylcyclopropyl)-2-(methoxymethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl)carbamate;
methyl-d3 ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-((1S,2S)-1-carbamoyl-2-methylcyclopropyl)-2-((R)-1-methoxyethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 ((1R,3R)-3-(6-((1-((1S,2S)-1-carbamoyl-2-methylcyclopropyl)-3-(methoxy-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d₃ ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl-d₃ ((1R,3R)-3-(6-((1-((1S,2S)-1-carbamoyl-2-methylcyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl-d₃ ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate;
methyl-d₃ ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate;
methyl-d₃ ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate;
methyl-d₃ ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(difluoromethyl)cyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1R,2S)-1-carbamoyl-2-methylcyclopropyl)-4-methyl-5-(methyl-d₃)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1R,2S)-1-carbamoyl-2-methylcyclopropyl)-4-methyl-5-(methyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl-d₃ (4-(6-((1-((1s,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)-2-fluorocyclopentyl)carbamate;
methyl (4-(6-((1-((1s,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)-2-fluorocyclopentyl)carbamate;
methyl-d₃ (3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate;
methyl-d₃ (3-(6-((1-((1R,2S)-2-carbamoyl-[1,1′-bi(cyclopropan)]-2-yl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate;
methyl (3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate; and
methyl ((1S,2S,4S)-4-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)-2-methoxycyclopentyl)carbamate;
   or a pharmaceutically acceptable salt thereof.

In some embodiments, the compound provided herein is methyl (3-(6-((1-(1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl (3-(6-((1-(1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((1-(1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((1-(1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((1-((1r,3R)-1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((1-((1r,3R)-1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((1-((1s,3S)-1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((1-((1s,3S)-1-carbamoyl-3-fluorocyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl (3-(6-((7-(1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl (3-(6-((7-(1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((7-(1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((7-(1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((7-((1s,3S)-1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((7-((1s,3S)-1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((7-((1r,3R)-1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((7-((1r,3R)-1-carbamoyl-3-fluorocyclobutyl)imidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl (3-(6-((1-(1-carbamoyl-2-methylcyclopropyl)-3-(2-oxopyrrolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl (3-(6-((1-(1-carbamoyl-2-methylcyclopropyl)-3-(2-oxopyrrolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((1-((1S,2S)-1-carbamoyl-2-methylcyclopropyl)-3-(2-oxopyrrolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((1-((1S,2S)-1-carbamoyl-2-methylcyclopropyl)-3-(2-oxopyrrolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl (3-(6-((1-(1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl (3-(6-((1-(1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl (3-(6-((1-(1-carbamoyl-2-methylcyclopropyl)-3-(2-hydroxypropan-2-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl (3-(6-((1-(1-carbamoyl-2-methylcyclopropyl)-3-(2-hydroxypropan-2-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((1-((1S,2S)-1-carbamoyl-2-methylcyclopropyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((1-((1S,2S)-1-carbamoyl-2-methylcyclopropyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate.

In some embodiments, the compound provided herein is methyl (3-(6-((1-(1-carbamoyl-2-methylcyclopropyl)-3-(dimethylcarbamoyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl (3-(6-((1-(1-carbamoyl-2-methylcyclopropyl)-3-(dimethylcarbamoyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate.

In some embodiments, the compound provided herein is methyl ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate, or a pharmaceutically acceptable salt thereof.

In some embodiments, the compound provided herein is methyl ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The term “Cₙ₋ₘ alkyl” is understood to include deuterated analogs of alkyl groups as defined herein, including but not limited to, groups such as trideuteromethyl (CD₃), pentadeuteroethyl (CD₂CD₃), and the like.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms. The term “Cₙ₋ₘ alkenyl” is understood to include deuterated analogs of alkenyl groups as defined herein, including but not limited to, groups such as trideuteroethenyl (—CD=CD₂), tetradeuteropropenyl, (—CD=CD-CD₂), and the like.

As used herein, “Cₙ₋ₘ alkynyl” refers to an alkyl group having one or more triple carbon-carbon bonds and having n to m carbons. Example alkynyl groups include, but are not limited to, ethynyl, propynyl (e.g., propyn-1-yl, propyn-2-yl, prop-2-yn-1-yl), and the like. In some embodiments, the alkynyl moiety contains 2 to 6, 2 to 4, or 2 to 3 carbon atoms. The term “Cₙ₋ₘ alkynyl” is understood to include deuterated analogs of alkynyl groups as defined herein, including but not limited to, groups such as deuteroethynyl (—C≡CD), trideuteropropyn-1-yl, (—C≡CCD₃), and the like.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. The term “Cₙ₋ₘ alkoxy” is understood to include deuterated analogs of the alkyl moiety of the alkoxy group as defined herein, including but not limited to, groups such as trideuteromethoxy (—OCD₃), pentadeuteroethoxy (—OCD₂CD₃), and the like.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl. The term “aryl” is understood to include deuterated analogs of the aryl groups as defined herein, including but not limited to, groups such as pentadeuterophenyl (i.e., perdeuterophenyl, phenyl-d₅), perdeuteronaphthyl, and the like.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms. The term “Cₙ₋ₘ haloalkoxy” is understood to include deuterated analogs of the haloalkyl moiety of the haloalkoxy group as defined herein, including but not limited to, groups such as deuterodifluoromethoxy (—OCDF₂), dideuterofluoromethoxy (—OCD₂F), and the like.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like. The term “Cₙ₋ₘ haloalkyl” is understood to include deuterated analogs of the haloalkyl group as defined herein, including but not limited to, groups such as deuterodifluoromethyl (—CDF₂), dideuterofluoromethyl (—CD₂F), and the like.

As used herein, the term “carbonyl”, employed alone or in combination with other terms, refers to a —C(O)— group.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carboxy” refers to a group of formula —C(O)OH.

As used herein, the term “di(Cₙ₋ₘ alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, azaspiro[2.4]heptanyl, and the like. In some embodiments, cycloalkyl is cyclopropyl, cyclobutyl, cyclopentyl, or cyclohexyl. The term “cycloalkyl” is understood to include deuterated analogs of the cycloalkyl groups as defined herein, including but not limited to, groups such as perdeuterocyclopropyl, perdeuterocyclobutyl, deuterocyclopentyl, pentadeuterocyclopentyl, perdeuterocyclopentyl, perdeuterocyclohexyl, and the like.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, pyrazolo[1,5-a]pyrimidinyl, indazolyl, imidazo[1,2-b]pyridazinyl, pyrazolo[1,5-a]pyrimidinyl, and the like. The term “heteroaryl” is understood to include deuterated analogs of the heteroaryl groups as defined herein, including but not limited to, groups such as perdeuteropyridinyl, perdeuteropyrazinyl, perdeuteropyrimidinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. The term “heterocycloalkyl” is understood to include deuterated analogs of the heterocycloalkyl groups as defined herein, including but not limited to, groups such as perdeuteroazetidinyl, perdeuteropyrrolidinyl, perdeuteropiperidinyl, and the like.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 5,6-dihydro-4H-pyrrolo[1,2-b]pyrazolyl, 3-oxopiperazinyl, oxo-pyrrolidinyl, oxo-pyridinyl, 2,3-dihydrobenzo[b][1,4]dioxinyl, 3,4-dihydro-2H-benzo[b][1,4]oxazinyl, octahydro-2H-pyrido[1,2-a]pyrazinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like. The terms “alkyl linking group” and “alkylene linking group” are understood to include deuterated analogs of the alkylene groups as defined herein.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula Ia, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as R-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected.

Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula (I) can be prepared, for example, using a process as illustrated in Scheme 1. In the process depicted in Scheme 1, the compounds of formula 1-1 can react with the compounds 1-2 via nucleophilic aromatic substitution reactions (e.g., in the presence of a base, such as N,N-diisopropylethylamine) followed by reduction of the nitro group in compound 1-3 (e.g. under reductive conditions, such as treatment with Zn powder and NH₄Cl) resulting in the formation of the compounds of formula 1-4. The compounds of formula 1-4 can be converted to cyclic urea 1-5 under standard conditions (e.g., in the presence of N-succinimidyl carbonate). The compounds of formula 1-5 can be further transformed to the compounds of formula 1-6 via methylation (e.g., in the presence of a methyl halide (e.g., iodomethane) and a base (e.g., cesium carbonate). The compounds of formula 1-6 can be transformed into compounds of formula 1-8 via NH deprotection of the protecting group (e.g., treatment with HCl or TFA) followed by carbamation (e.g., in the presence of methyl chloroformate and a base). The compounds of formula 1-7 can be used to prepare compounds of formula 1-8 through Buchwald-Hartwig cross-coupling reactions (e.g., in the presence of tris(dibenzylideneacetone)dipalladium(0), Xantphos and cesium carbonate) followed by NH deprotection of the protecting group (e.g., treatment with HCl or TFA). Then, the compounds of formula 1-8 can be coupled with the compounds of formula 1-9, where X is a halogen (e.g., Cl, or Br), under Buchwald-Hartwig cross-coupling reactions (e.g., in the presence of tris(dibenzylideneacetone)dipalladium(0), Xantphos and cesium carbonate) to afford the compounds of Formula I.

Alternatively, the compounds of formula 1-7 can directly join together with the compounds of formula 1-10 via Buchwald-Hartwig cross-coupling reactions (e.g., in the presence of tris(dibenzylideneacetone)dipalladium(0), Xantphos and cesium carbonate) to yield the compounds of Formula I.

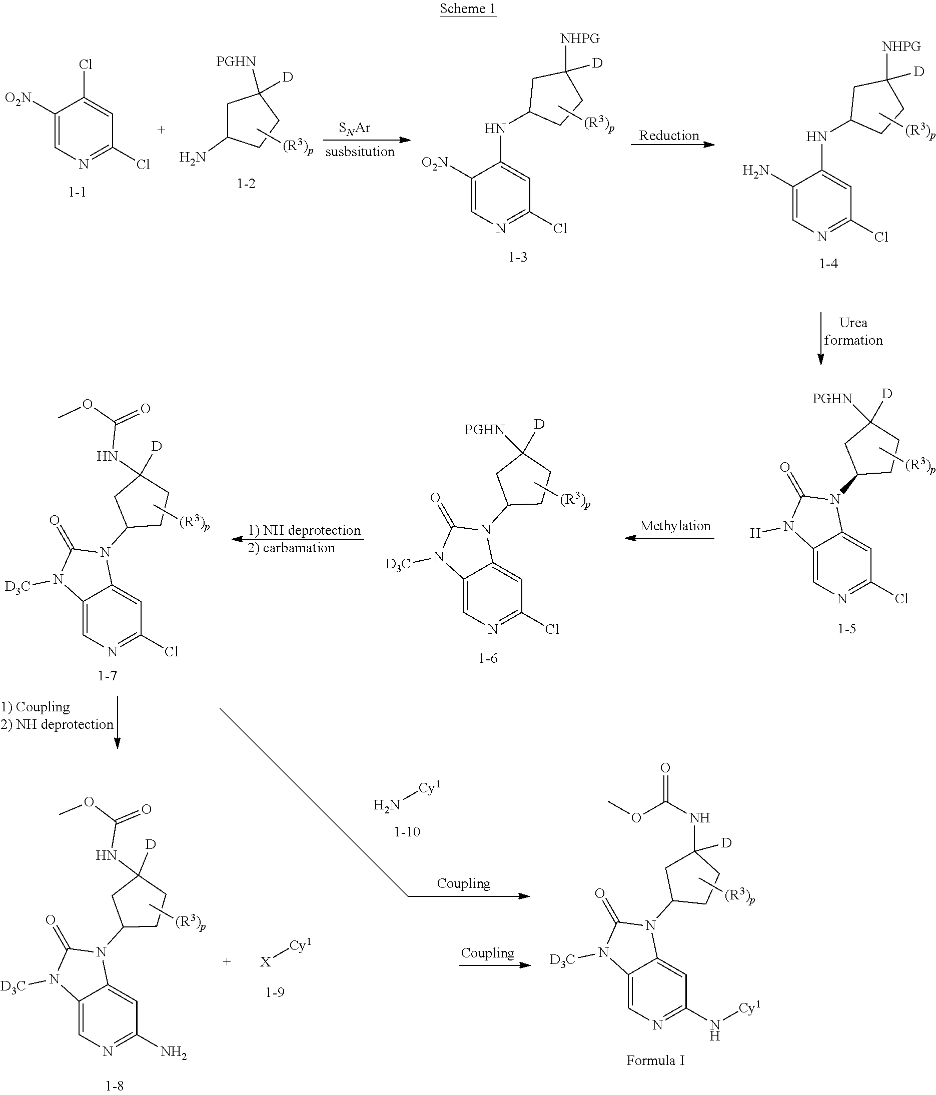

When Cy¹ is imidazo[1,5-b]pyridazine ring, the compounds of formula 1-9 and 1-10 can be prepared using process as illustrated in Scheme 2. The compounds of formula 2-1 can undergo peptide coupling with acids 2-2 under standard reaction conditions (e.g., in the presence of a coupling reagent such as HATU and a suitable base), followed by cyclization reaction (e.g., in the presence POCl₃) on the compounds of formula 2-3 to provide the compounds of formula 2-4 (1-9). After functional group manipulations (e.g., iodination, cross-coupling etc.) on the compound of formula 2-4, the compounds of formula 2-5 can be prepared. The compounds of formula 2-5 can be elaborated into the compounds of formula 1-10 through NH deprotection of the protecting group (e.g., treatment with TFA).

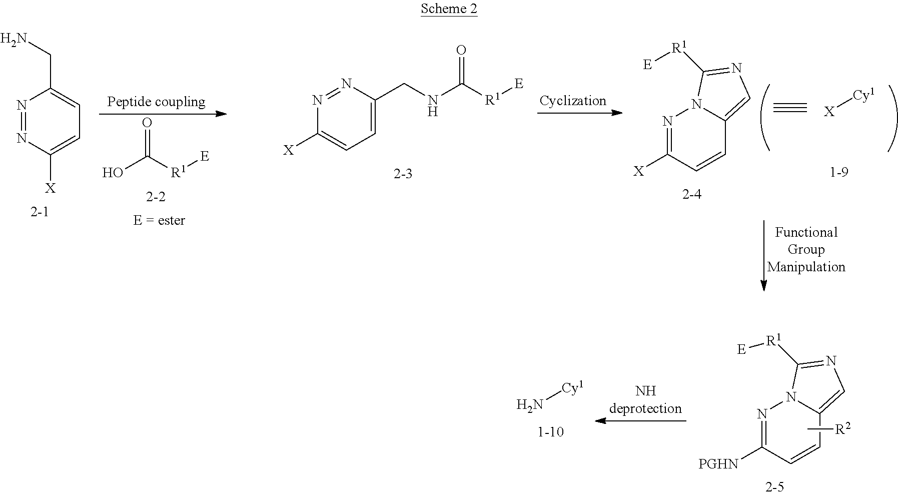

When Cy¹ is 3H-imidazo[4,5-b]pyridine ring, the compounds of formula 1-9 can be prepared using a process as illustrated in Scheme 3. The compounds of formula 3-1, where L₁ is a halogen (e.g., F, Cl, or Br), can react with the compounds 3-2 via nucleophilic aromatic substitution reactions (e.g., in the presence of a base, such as N,N-diisopropylethylamine) to give the compounds of formula 3-3. The reduction of the nitro group in compound of formula 3-3 (e.g. under reductive conditions, such as treatment with Zn powder and NH₄Cl) followed by cyclization reaction (e.g. in the presence of ethyl orthoformate) yield the compounds of formula 3-4. After functional group manipulations (e.g., oxidation, methylation etc.) on the compound of formula 3-4, the compounds of formula 1-9 can be prepared.

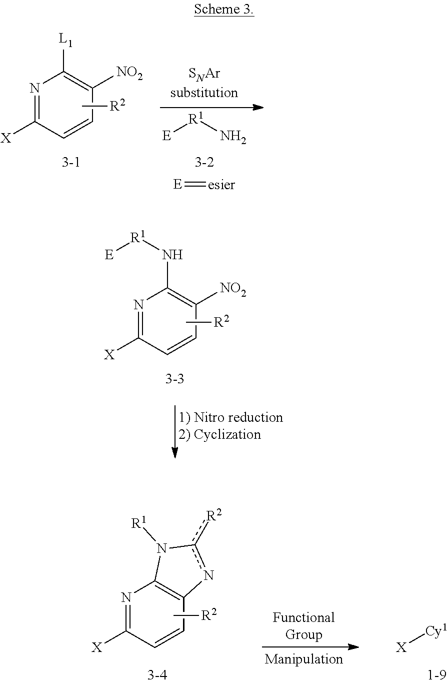

When Cy¹ is 1H-pyrazolo[3,4-b]pyridine ring, the compounds of formula 1-9 can be prepared using a process as illustrated in Scheme 4. The compounds of formula 4-1, where L₁ is a halogen (e.g., F, Cl, or Br), can react with the compounds of formula 4-2 via nucleophilic aromatic substitution reactions (e.g., in the presence of a base, such as N,N-diisopropylethylamine) to give the compounds of formula 4-3. The cyclization of compound of formula 4-3 (e.g. treatment with LHMDS, then (aminooxy)diphenylphosphine oxide) followed by de-amination reaction (e.g. in the presence of tert-butyl nitrite) yield the compounds of formula 4-4. After functional group manipulations (e.g., cross-coupling, hydrogenation etc.) on the compound of formula 4-4, the compounds of 1-9 can be prepared.

Alternatively, the compounds of formula 4-4 can be also prepared via direct condensation-cyclization (e.g., heating in the presence of a base, such as N,N-diisopropylethylamine) between the compounds of formula 4-5, where L₁ is a halogen (e.g., F, Cl, or Br), and the compounds of formula 4-6.

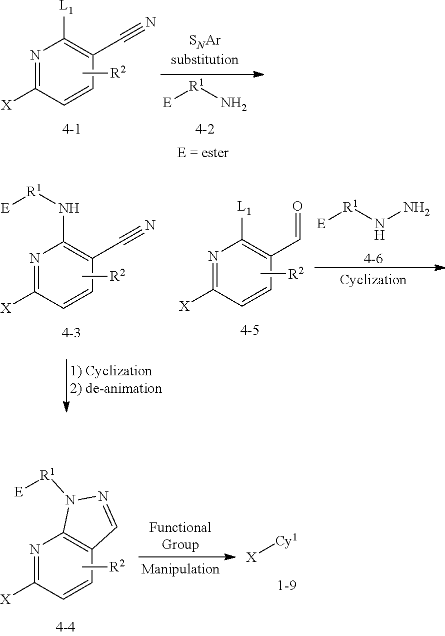

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can inhibit the activity of the V617F variant of the protein-tyrosine kinase JAK2 (i.e., “V617F” or “JAK2V617F”). Compounds which inhibit V617F are useful in providing a means of preventing the growth or inducing apoptosis in tumors, particularly by inhibiting angiogenesis. It is therefore anticipated that the compounds of the disclosure are useful in treating or preventing proliferative disorders such as cancers. In particular tumors with activating mutants of receptor tyrosine kinases or upregulation of receptor tyrosine kinases may be particularly sensitive to the inhibitors.

As disclosed herein, the compounds of the invention exhibit unexpectedly improved properties (e.g., improved potency, PK properties, and the like) compared to compounds disclosed in U.S. patent application Ser. No. 18/602,434, the disclosure of which is incorporated herein by reference in its entirety. The following compounds from U.S. Ser. No. 18/602,434 are provided herein as Comparative Examples A-C:

| Comparative Example | Structure |
|---|---|
| Comparative Example A | 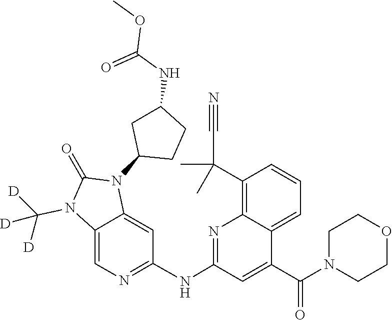 [SMILES: [2H]C([2H])([2H])n1c(=O)n([C@@H]2CC[C@@H](NC(=O)OC)C2)c2cc(Nc3cc(C(=O)N4CCOCC4)c4cccc(C(C)(C)C#N)c4n3)ncc21] |
| Comparative Example B | 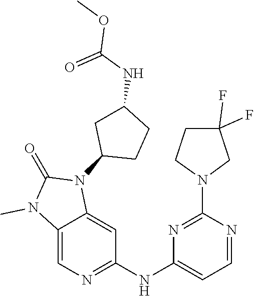 [SMILES: COC(=O)N[C@@H]1CC[C@@H](n2c(=O)n(C)c3cnc(Nc4ccnc(N5CCC(F)(F)C5)n4)cc32)C1] |
| Comparative Example C | 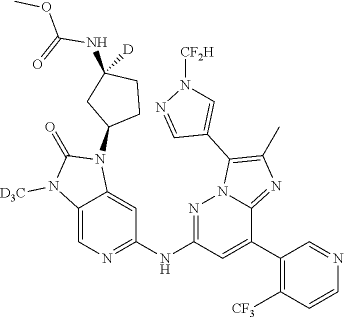 [SMILES: [2H]C([2H])([2H])n1c(=O)n([C@@H]2CC[C@]([2H])(NC(=O)OC)C2)c2cc(Nc3cc(-c4cnccc4C(F)(F)F)c4nc(C)c(-c5cnn(C(F)F)c5)n4n3)ncc21] |

In certain embodiments, the disclosure provides a method for treating a V617F-related disorder in a patient in need thereof, comprising the step of administering to said patient a compound of the disclosure, or a pharmaceutically acceptable composition thereof.

Myeloproliferative diseases (MPD) are multipotent hematopoietic stem cell disorders characterized by excess production of various blood cells. MPNs include polycythemia vera (PV), essential thrombocythemia (ET), and idiopathic myelofibrosis (IMF). JAK2 V617F mutation is reported in about 95% of patients with PV, in 35% to 70% of patients with ET, and 50% of patients with IMF. Also, JAK2 exon 12 mutations are detected in some of the V617F-negative PV patients (Ma et al., J. Mol. Diagn., 11: 49-53, 2009). In some embodiments, the compounds of the disclosure can be useful in the treatment of myeloproliferative disorders (e.g., myeloproliferative neoplasms) in a patient in need thereof, such as polycythemia vera, essential thrombocythemia, myelofibrosis with myeloid metaplasia (MMM), primary myelofibrosis (PMF), chronic myelogenous leukemia (CML), chronic myelomonocytic leukemia (CMML), hypereosinophilic syndrome (HES), systemic mast cell disease (SMCD), and the like.

In some embodiments, the myeloproliferative disorder is selected from polycythemia vera, essential thrombocythemia, myelofibrosis with myeloid metaplasia, primary myelofibrosis, post-essential thrombocythemia myelofibrosis, and post polycythemia vera myelofibrosis.

In some embodiments, the myeloproliferative disorder is a myeloproliferative neoplasm.

In some embodiments, the myeloproliferative disorder is myelofibrosis (e.g., primary myelofibrosis (PMF) or post polycythemia vera/essential thrombocythemia myelofibrosis (Post-PV/ET MF)).

In some embodiments, the myeloproliferative disorder is primary myelofibrosis (PMF).

In some embodiments, the myeloproliferative disorder is post-essential thrombocythemia myelofibrosis (Post-ET MF).

In some embodiments, the myeloproliferative disorder is post polycythemia vera myelofibrosis (Post-PV MF).

In some embodiments, the myeloproliferative disorder is selected from primary myelofibrosis (PMF), polycythemia vera (PV), and essential thrombocythemia (ET).

In some embodiments, the myeloproliferative neoplasm is primary myelofibrosis (PMF).

In some embodiments, the myeloproliferative neoplasm is polycythemia vera (PV).

In some embodiments, the myeloproliferative neoplasm is essential thrombocythemia (ET).

Myeloproliferative diseases include disorders of a bone marrow or lymph node-derived cell type, such as a white blood cell. A myeloproliferative disease can manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CML), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

For example, the compounds of the disclosure are useful in the treatment of cancer. Example cancers include bladder cancer (e.g., urothelial carcinoma, squamous cell carcinoma, adenocarcinoma), breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma (e.g., intrahepatic, hilar or perihilar, distal extrahepatic), liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g. exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

Further example cancers include hematopoietic malignancies such as leukemia or lymphoma, multiple myeloma, chronic lymphocytic lymphoma, adult T cell leukemia, acute myeloid leukemia (AML), B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera (PV), essential thrombocythemia (ET), and primary myelofibrosis (PMF)), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphomas, and Burkitt's lymphoma.

In certain embodiments, provided herein is a method of treating cancer comprising administering to a patient in need thereof a therapeutically effect amount of a compound of the disclosure. In certain embodiments, the cancer is selected from T lymphoblastic lymphoma, glioblastoma, melanoma, rhabdosarcoma, lymphosarcoma, and osteosarcoma.

Other cancers treatable with the compounds of the disclosure include tumors of the eye, glioblastoma, melanoma, leiomyosarcoma, and urothelial carcinoma (e.g., ureter, urethra, bladder, urachus).

The compounds of the disclosure can also be useful in the inhibition of tumor metastases.

In some embodiments, the compounds of the disclosure as described herein can be used to treat Alzheimer's disease, HIV, or tuberculosis.

In some embodiments, the compounds of the disclosure can be useful in the treatment of myelodysplastic syndrome (MDS) in a patient in need thereof. In some embodiments, said patient having the myelodysplastic syndrome (MDS) is red blood cell transfusion dependent.

As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥10% of 1 cell |
| unilineage dysplasia | | line, <5% blasts |
| (RCUD) | | |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × 10⁹/L | Dysplasia in ≥10% of cells |
| multilineage dysplasia | monocytes | in ≥2 hematopoietic lineages, ±15% |
| | | ring sideroblasts, <5% blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% to |
| | monocytes | 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ±Auer rods, 10% to |
| | monocytes | 19% blasts |
| Myelodysplastic syndrome, | Cytopenias | Unilineage or no dysplasia but |
| unclassified (MDS-U) | | characteristic MDS |
| | | cytogenetics, <5% blasts |
| MDS associated with | Anemia, platelets normal | Unilineage erythroid. Isolated |
| isolated del(5q) | or increased | del(5q), <5% blasts |

In some embodiments, the myelodysplastic syndrome is refractory cytopenia with unilineage dysplasia (RCUD).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del(5q).

In some embodiments, the myelodysplastic syndrome is refractory to erythropoiesis-stimulating agents.

In some embodiments, the compounds of the disclosure can be useful in the treatment of myeloproliferative disorder/myelodysplastic overlap syndrome (MPD/MDS overlap syndrome).

In some embodiments, the compounds of the disclosure can be useful in the treatment of leukemia.

In some embodiments, the compounds of the disclosure can be useful in the treatment of acute myeloid leukemia (AML).

In addition to oncogenic neoplasms, the compounds of the disclosure can be useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes.

The compounds provided herein may further be useful in the treatment of fibrotic diseases, such as where a disease symptom or disorder is characterized by fibrosis. Example fibrotic diseases include liver cirrhosis, glomerulonephritis, pulmonary fibrosis, systemic fibrosis, rheumatoid arthritis, and wound healing.

In some embodiments, the compounds provided herein can be used in the treatment of a hypophosphatemia disorder such as, for example, X-linked hypophosphatemic rickets, autosomal recessive hypophosphatemic rickets, and autosomal dominant hypophosphatemic rickets, or tumor-induced osteromalacia.

In some embodiments, the compounds provided herein may further be useful for the treatment of clonal hematopoietic disorders, and related disorders. As used herein, “clonal hematopoiesis” refers to the presence of clonal populations of hematopoietic stem cells (HSC). Hematopoiesis is generally a polyclonal process with HSCs of equipotential, giving rise to erythroid, lymphoid, myeloid, or megakaryocytic cells. Mutations may occur in genes that confer selective fitness advantage with aging HSCs less adept to correct for these errors, giving rise to clonally expanded populations of stem cells.

The presence of mutations can lead to clonal hematopoiesis of indeterminate potential (CHIP), which may induce an altered inflammatory state that is associated with an increased risk of cardiovascular conditions, coronary artery disease, ischemic stroke, atherosclerosis, poorer outcomes in aortic stenosis and heart failure, and enhanced thrombogenesis.

Example diseases and disorders associated with clonal hematopoiesis include, but are not limited to, autoimmune diseases, atherosclerosis, cancer, Philadelphia-negative myeloproliferative neoplasm (MPN), polycythaemia vera (PV), essential thrombocythaemia (ET), eye disorders, chronic obstructive pulmonary disease (COPD), osteoporosis, chronic liver disease, acute kidney injury, thoracic aortic aneurysms, NETosis, NETosis-related conditions, thrombosis, cardiac diseases and disorders, myocardial infarction (MI), ulcerative colitis, inflammatory bowel disease, pericarditis, myocarditis, thromboembolis, myelodysplastic syndrome (MDS), myeloproliferative neoplasms (MPNs), chronic infections, mycobacterial infection, sepsis, pneumonia, HIV, non-Hodgkin's lymphoma, glioma, and acute myeloid leukemia.

In some embodiments, the methods provided herein comprises treating a disease or disorder associated with clonal hematopoiesis selected from autoimmune diseases, atherosclerosis, cancer, Philadelphia-negative myeloproliferative neoplasm (MPN), polycythaemia vera (PV), essential thrombocythaemia (ET), eye disorders, chronic obstructive pulmonary disease (COPD), osteoporosis, chronic liver disease, acute kidney injury, thoracic aortic aneurysms, NETosis, NETosis-related conditions, thrombosis, venous thromboembolism, cardiac diseases and disorders, myocardial infarction (MI), ulcerative colitis, inflammatory bowel disease, pericarditis, myocarditis, thromboembolis, myelodysplastic syndrome (MDS), myeloproliferative neoplasms (MPNs), chronic infections, mycobacterial infection, sepsis, pneumonia, HIV, non-Hodgkin's lymphoma, glioma, and acute myeloid leukemia.

As used herein, “clonal hematopoiesis of indeterminate potential (CHIP)” refers to refers to CH specifically harboring a somatic mutation in a myeloid neoplasm driver gene with a variant allele frequency (VAF)≥2% in a patient lacking a hematologic neoplasm or unexplained cytopenia. In some embodiments, the method provided herein comprises treating a disease or disorder associated with clonal hematopoiesis of indeterminate potential (CHIP).

In some embodiments, the clonal hematopoiesis of indeterminate potential is JAK2 CHIP.

In some embodiments, the clonal hematopoiesis of indeterminate potential is JAK2 V617F CHIP.

In some embodiments, the clonal hematopoiesis of indeterminate potential (CHIP) is clonal cytopenia of undetermined significance (CCUS).

As used herein, “clonal cytopenia of undetermined significance (CCUS)” refers to CHIP detected in the presence of one or more persistent unexplained cytopenias, while diagnostic criteria for any defined myeloid neoplasm are not met.

In some embodiments, the method provided herein comprises treating a disease or disorder associated with clonal cytopenia of undetermined significance (CCUS).

In some embodiments, the method further comprises identifying the patient who has had at least one indication event; determining the presence of the mutant JAK2 V617F in the patient; selecting a patient having the mutant JAK2 V617F; and administering to the patient an effective amount of a compound provided herein, or a pharmaceutically acceptable salt thereof. In some embodiments, the patient has been identified (e.g., diagnosed) as having clonal hematopoiesis of indeterminate potential (CHIP).

The methods described herein include methods for the treatment of clonal hematopoiesis of indeterminate potential (CHIP) patients (e.g., patients diagnosed as having CHIP) who have one or more of the following diseases or disorders, or who are at higher risk of developing one or more of the following diseases or disorders: autoimmune diseases (e.g., antineutrophil cytoplasmic antibody (ANCA)-associated vasculitis (AAV), systemic lupus erythematosus (SLE), rheumatoid arthritis (RA), psoriasis, antiphospholipid syndrome (APS), multiple sclerosis (MS), dermatomyositis (DM), polymyositis (PM), IgG4-related autoimmune pancreatitis (AIP), drug-induced autoimmune diseases (see e.g., He et al., Chin. Med. J. (Engl). 2018, Jul. 5; 131(13):1513-1519; Lee et al., Autoimmun. Rev. 2017 November; 16(11):1160-1173)); atherosclerosis; cancer (see e.g., Olsson and Cedarvall, Front Immunol. 2016; 7: 373; Cedarvall and Olsson, Oncoscience, 2015; 2(11): 900-901; Genovse et al, N. Engl. J. Med. 2014; 371:2477-2487; Cordua et al, Blood, 2019; 134(5):469-479); Philadelphia-negative myeloproliferative neoplasm (MPN), e.g., polycythaemia vera (PV) or essential thrombocythaemia (ET); eye disorders, such as drusen and age-related macular degeneration (see e.g., EClinicalMedicine, 2022; 43: 101248); chronic obstructive pulmonary disease (COPD) (see e.g., Miller et al, Blood, 2022; 139:357-6839); osteoporosis (see e.g, Kim et al. J. Exp. Med., 2021, 218:e2021187); chronic liver disease (see e.g., Wong et al., Nature, 2023, 616(7958):747-754); acute kidney injury (see e.g., Vlasschaert et al., Nature Med. 2024; 30: 810-817); and thoracic aortic aneurysms (see e.g., Nakao et al., J. Am. Coll. Cardiol. 2023; 81(21): 2128-2130).

In some embodiments, the methods provided herein can be used, e.g., for reducing NETosis, or reducing the risk of NETosis-related conditions, e.g., treating or reducing the risk of occurrence or reoccurrence of a cardiovascular event (e.g., thrombosis or myocardial infarction (MI)). Generally, the methods include administering a therapeutically effective amount of an inhibitor of the present disclosure, to a patient who is in need of, or who has been determined to be in need of, such treatment. In some embodiments, the methods described herein further comprise determining the patient's Jak2 Genotype, i.e., determining whether the patient has one or more JAK2V617F/I alleles, and selecting and treating patients who do have one or more JAK2V617F/I alleles. In some embodiments, the patient does not have one or more JAK2V617F/I alleles.

In some embodiments, the method comprises treating a JAK2 V617F CHIP-induced disease in the patient.

In some embodiments, the JAK2 V617F CHIP-induced disease is selected from hematologic cancer, myeloproliferative neoplasm, myeloid cancer, coronary heart disease, stroke, ischemic heart disease, venous thromboembolism, pulmonary embolism, drusen, age-related macular degeneration, chronic obstructive pulmonary disease (COPD), osteoporosis, chronic liver disease, acute kidney injury, thoracic aortic aneurysm, leukocytosis, neutropenia, hyperplasia, arteriosclerosis, phlebosclerosis caused by activated neutrophils, aortic aneurysm, hypertension, pulmonary hypertension, myeloid leukemia, lymphocytic leukemia, myelodysplastic syndrome, aplastic anemia, paroxysmal nocturnal hemoglobinuria, malignant lymphoma, and multiple myeloma.

In some embodiments, the CHIP-induced disease is selected from acute kidney injury, chronic liver disease, thoracic aortic aneurysm, coronary heart disease, and hematological malignancy.

In some embodiments, the CHIP-induced disease is acute kidney injury.

In some embodiments, the CHIP-induced disease is chronic liver disease.

In some embodiments, the CHIP-induced disease is thoracic aortic aneurysm.

In some embodiments, the CHIP-induced disease is coronary heart disease.

In some embodiments, the CHIP-induced disease is a hematological malignancy.

In some embodiments, the method comprises treating JAK2 V617F CHIP-induced thrombosis in the patient.

In some embodiments, the JAK2 V617F CHIP-induced thrombosis comprises arterial thrombosis.

In some embodiments, the JAK2 V617F CHIP-induced thrombosis comprises venous thrombosis.

In some embodiments, the method comprises treating JAK2 V617F CHIP-induced atherosclerosis in the patient.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a V617F variant with a compound described herein includes the administration of a compound described herein to an individual or patient, such as a human, having a V617F variant, as well as, for example, introducing a compound described herein into a sample containing a cellular or purified preparation containing the V617F variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein for treatment or prevention of V617F-associated diseases, disorders or conditions, or diseases or conditions as described herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein can be used in combination with one or more inhibitors of the enzyme or protein receptors such as HPK1, SBLB, TUT4, A2A/A2B, CD19, CD47, CDK2, STING, ALK2, LIN28, ADARI, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds described herein can be used in combination with targeted therapies, including JAK kinase inhibitors (ruxolitinib, additional JAK1/2 and JAK1-selective, baricitinib or itacitinib), Pim kinase inhibitors (e.g., LGH447, INCB053914 and SGI-1776), PI3 kinase inhibitors including PI3K-delta selective and broad spectrum PI3K inhibitors (e.g., INCB50465 and INCB50797), PI3K-gamma inhibitors such as PI3K-gamma selective inhibitors, MEK inhibitors, CSF1R inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibiors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, compounds described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US 20170107216, US 20170145025, US 20170174671, US 20170174679, US 20170320875, US 20170342060, US 20170362253, and US 20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is retifanlimab (also known as MGA012), nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is retifanlimab. In some embodiments, the anti-PD1 antibody is SHR-1210. Other anti-cancer agent(s) include antibody therapeutics such as 4-1BB (e.g. urelumab, utomilumab.

In some embodiments, the compounds of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the compounds described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with compounds described herein for the treatment of cancer include chemotherapeutic agents, targeted cancer therapies, immunotherapies or radiation therapy. Compounds described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with inhibitors described herein. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds described herein. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein. In some embodiments, the JAK inhibitor is selective for JAK1 over JAK2 and JAK3.

Other suitable agents for use in combination with compounds described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (e.g., vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel, mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure can be used in combination with bone marrow transplant for the treatment of a variety of tumors of hematopoietic origin (see e.g., U.S. Pat. Nos. 9,233,985, 10,065,974, 10,287,303, 8,524,867, the disclosures of which are incorporated by reference herein in their entireties).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating V617F in tissue samples, including human, and for identifying V617F inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes V617F assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ (i.e., trideuteromethyl) being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

In some embodiments, the compound provided herein (e.g., the compound of any of Formulas I-XVIab), or a pharmaceutically acceptable salt thereof, comprises at least one deuterium atom.

In some embodiments, the compound provided herein (e.g., the compound of any of Formulas I-XVIab), or a pharmaceutically acceptable salt thereof, comprises two or more deuterium atoms.

In some embodiments, the compound provided herein (e.g., the compound of any of Formulas I-XVIab), or a pharmaceutically acceptable salt thereof, comprises three or more deuterium atoms.

In some embodiments, for a compound provided herein (e.g., the compound of any of Formulas I-XVIab), or a pharmaceutically acceptable salt thereof, all of the hydrogen atoms are replaced by deuterium atoms (i.e., the compound is “perdeuterated”).

In some embodiments, the cyclopentyl group of the compounds of Formulas I-XVIab comprises 0, 1, 2, 3, 4, or 5 deuterium atoms, for example, as shown below in Formulas A-H:

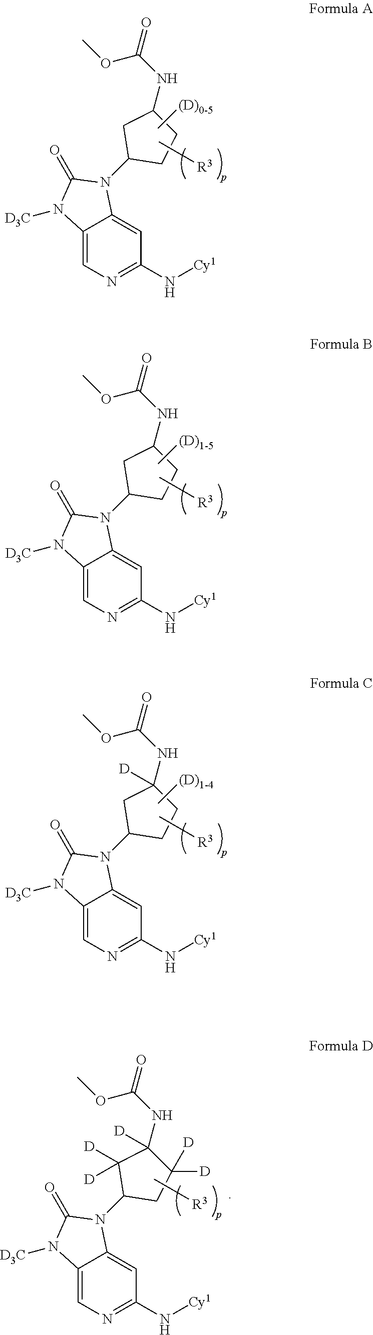

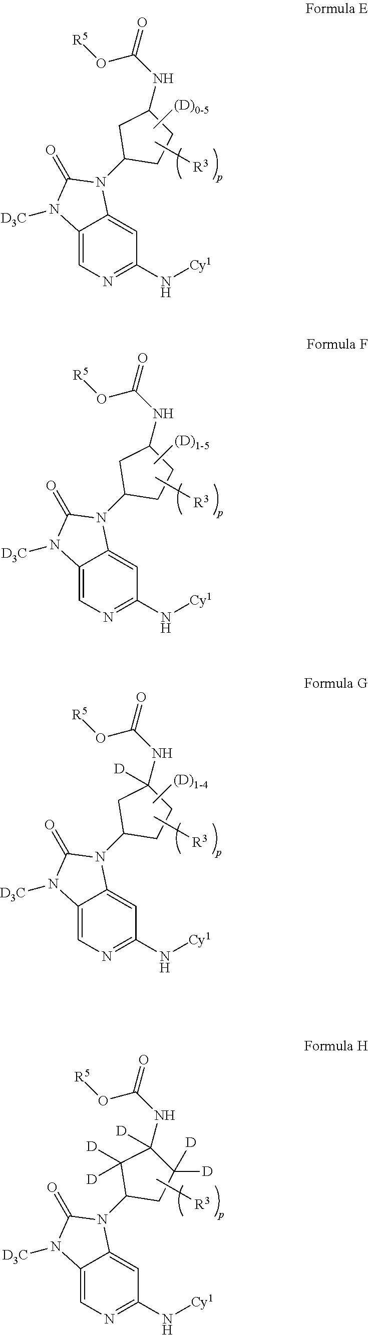

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro V617F labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind V617F by monitoring its concentration variation when contacting with V617F, through tracking of the labeling. For example, a test compound (labeled) can be evaluated for its ability to reduce binding of another compound which is known to bind to V617F (i.e., standard compound). Accordingly, the ability of a test compound to compete with the standard compound for binding to V617F directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of V617F-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)).

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J Comb. Chem., 6, 874-883 (2004)).

pH=10 purifications: Waters XBridge™ C₁₈ 5 μm, 30×100 mm column, eluting with mobile phase A: 0.10% NH₄OH in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

### Intermediate A. Methyl ((1R,3R)-3-(6-amino-3-(methyl-d₃)-2-oxo-2,3-dihydro-1H-imidazo[4,5-c]pyridin-1-yl)cyclopentyl-1-d)carbamate

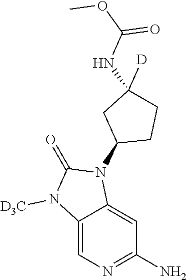 [SMILES: [2H]C([2H])([2H])n1c(=O)n([C@@H]2CC[C@@]([2H])(NC(=O)OC)C2)c2cc(N)ncc21]

### Step 1. tert-Butyl ((1R,3S)-3-hydroxycyclopentyl-3-d)carbamate

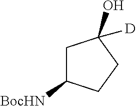 [SMILES: [2H][C@]1(O)CC[C@@H](NC(=O)OC(C)(C)C)C1]

To a flask containing tert-butyl (R)-(3-oxocyclopentyl)carbamate (10.0 g, 50.2 mmol) in MeOH (100 mL) at 0° C. was added sodium borodeuteride (1.26 g, 30.1 mmol) in portions (over 10 times) during 30 min. The reaction mixture was stirred at 0° C. for 3 h. The reaction mixture was concentrated, diluted with DCM (250 mL), and washed with 1 M sodium hydroxide solution. After extraction with DCM (100 mL) for 3 times, the organic layers were combined, dried over MgSO₄ and concentrated in vacuo. A small portion of this crude material was directly used in the synthesis of Intermediate B as a mixture of diastereomers. The rest of the crude material was purified by CombiFlash (earlier peak) to give the desired product as a single isomer (white solid). LCMS calculated for C₆H₁₁DNO₃ (M+H-t-Bu)⁺: m/z=147.1; found 147.1.

### Step 2. tert-Butyl ((1R,3R)-3-(1,3-dioxoisoindolin-2-yl)cyclopentyl-3-d)carbamate

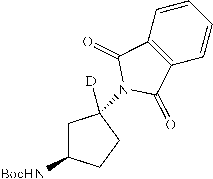 [SMILES: [2H][C@@]1(N2C(=O)c3ccccc3C2=O)CC[C@@H](NC(=O)OC(C)(C)C)C1]

To a flask containing tert-butyl ((1R,3S)-3-hydroxycyclopentyl-3-d)carbamate (3.98 g, 19.7 mmol) in THF (79 ml) was added triphenylphosphine (6.19 g, 23.61 mmol) and isoindoline-1,3-dione (4.34 g, 29.5 mmol). The reaction mixture was cooled down to 0° C. and the diisopropyl (E)-diazene-1,2-dicarboxylate (4.77 g, 4.65 mL, 23.61 mmol) was added dropwise to the flask. The reaction mixture was stirred at 0° C. to r.t for 3 h. The reaction mixture was quenched with water (30 mL) and extraction with DCM. The organic layer was combined, dried over MgSO₄ and concentrated in vacuo. The crude material was purified by Biotage Isolera. LCMS calculated for C₁₈H₂₂DN₂O₄(M+H)⁺: m/z=332.2; found 332.1.

### Step 3. tert-Butyl ((1R,3R)-3-aminocyclopentyl-3-d)carbamate

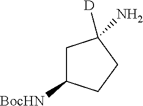 [SMILES: [2H][C@@]1(N)CC[C@@H](NC(=O)OC(C)(C)C)C1]

To a flask containing tert-butyl ((1R,3R)-3-(1,3-dioxoisoindolin-2-yl)cyclopentyl-3-d)carbamate (5.5 g, 16.60 mmol) in ethanol (55.3 mL) was added hydrazine (1.06 g, 1.0 mL, 33.2 mmol). The reaction mixture was stirred at 80° C. for 1.5 h with white solid generation during the reaction. After cooling down to r.t, the reaction mixture was filtered and washed with ethanol (100 mL) and DCM (100 mL) to filter out the white solid as side product. The filtrate was collected and concentrated in vacuo to give the crude product. The obtained crude product was used in the next step without further purification. LCMS calculated for C₆H₁₂DN₂O₂(M+H-t-Bu)⁺: m/z=146.1; found: 146.1.

### Step 4. Methyl ((1R,3R)-3-aminocyclopentyl-1-d)carbamate

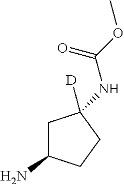

To a flask containing tert-butyl ((1R,3R)-3-aminocyclopentyl-3-d)carbamate (5.0 g, 24.8 mmol) and DIPEA (3.53 g, 4.77 mL, 27.3 mmol) in DCM (200 mL) and MeOH (5 mL) at 0° C. was added methyl chloroformate (1.92 mL, 24.8 mmol) dropwise. After stirring at 0° C. for 10 min., the reaction was quenched with MeOH (10 mL) and concentrated in vacuo. The residue was re-dissolved in DCM (200 mL) and washed with sodium bicarbonate saturated solution, dried over MgSO₄ and concentrated in vacuo. The crude material was treated with 4 N HCl in 1,4-dioxane (20 mL). After stirring at r.t for 1 h, the reaction mixture was concentrated in vacuo. The obtained crude product as HCl salt was used in the next step without further purification. LCMS calculated for C₇H₁₄DN₂O₂(M+H)⁺: m/z=160.1; found: 160.1.

### Step 5. Methyl ((1R,3R)-3-((2-chloro-5-nitropyridin-4-yl)amino)cyclopentyl-1-d)carbamate

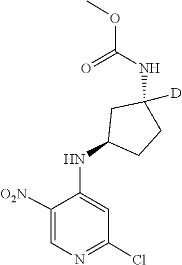 [SMILES: [2H][C@@]1(NC(=O)OC)CC[C@@H](Nc2cc(Cl)ncc2[N+](=O)[O-])C1]

To a vial containing methyl ((1R,3R)-3-aminocyclopentyl-1-d)carbamate (1.60 g, 10.0 mmol) in acetonitrile (1.5 mL) was added DIPEA (4.4 mL, 25.1 mmol) and 2,4-dichloro-5-nitropyridine (1.94 g, 10.0 mmol). The reaction mixture was stirred at 60° C. for 4 h. After cooling down to r.t., the reaction mixture was diluted with water (20 mL) and EtOAc (50 mL). The layers were separated, and the aqueous layer was further extracted with EtOAc (3×50 mL). The combined organic layers were dried over Na₂SO₄, filtered, and concentrated in vacuo. The obtained crude product was used in the next step without further purification. LCMS calculated for C₁₂H₁₅DClN₄O₄(M+H)⁺: m/z=316.1; found: 316.1.

### Step 6. Methyl ((1R,3R)-3-((5-amino-2-chloropyridin-4-yl)amino)cyclopentyl-1-d)carbamate

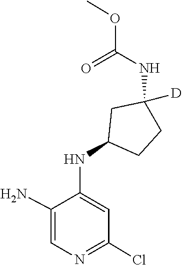 [SMILES: [2H][C@@]1(NC(=O)OC)CC[C@@H](Nc2cc(Cl)ncc2N)C1]

A round-bottom flask containing zinc powder (5.3 g, 80 mmol) and ammonium chloride (4.30 g, 80 mmol) in MeOH (100 mL) was placed in an ice-water bath at 0° C. A solution of tert-butyl ((1R,3R)-3-((2-chloro-5-nitropyridin-4-yl)amino)cyclopentyl-1-d)carbamate (2.8 g, 8.9 mmol) in 30 mL (CH₂Cl₂:MeOH=5:1) was added via addition funnel to the zinc power suspension at the rate of 1 drop/sec with vigorous stirring. After stirring at r.t for 3 h, the reaction mixture was filtered and washed with CH₂Cl₂ (40 mL) for three times. The filtrate was collected and concentrated. The residue was re-dissolved in CH₂Cl₂ (200 mL), washed with water (20 mL) and extracted with CH₂Cl₂. The organic phase was washed with brine, dried over MgSO₄ and concentrated in vacuo. The obtained crude product was used in the next step without further purification. LCMS calculated for C₁₂H₁₇DCNl₄O₂ (M+H)⁺: m/z=286.1; found 286.2.

### Step 7. Methyl ((1R,3R)-3-(6-chloro-2-oxo-2,3-dihydro-1H-imidazo[4,5-c]pyridin-1-yl)cyclopentyl-1-d)carbamate

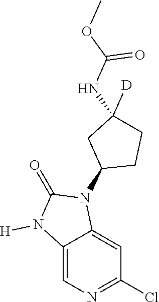 [SMILES: [2H][C@@]1(NC(=O)OC)CC[C@@H](n2c(=O)[nH]c3cnc(Cl)cc32)C1]

To a flask containing methyl ((1R,3R)-3-((5-amino-2-chloropyridin-4-yl)amino)cyclopentyl-1-d)carbamate (2.0 g, 7.0 mmol) in acetonitrile (20 mL) was added bis(2,5-dioxopyrrolidin-1-yl) carbonate (2.0 g, 7.7 mmol) in portions (over 10 times) during 30 min. After stirring at r.t for 16 h, the reaction mixture was concentrated in vacuo and washed with aqueous saturated sodium bicarbonate solution and extracted with CH₂Cl₂. The organic phase was washed with brine, dried over MgSO₄ and concentrated in vacuo. The obtained crude product was used in the next step without further purification. LCMS calculated for C₁₃H₁₅DClN₄O₃(M+H)⁺: m/z=312.1; found: 312.1.

### Step 8. Methyl ((1R,3R)-3-(6-chloro-3-(methyl-d₃)-2-oxo-2,3-dihydro-1H-imidazo[4,5-c]pyridin-1-yl)cyclopentyl-1-d)carbamate

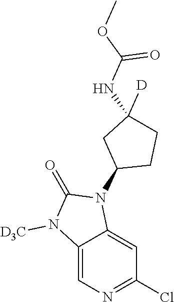 [SMILES: [2H]C([2H])([2H])n1c(=O)n([C@@H]2CC[C@@]([2H])(NC(=O)OC)C2)c2cc(Cl)ncc21]

To a flask containing methyl ((1R,3R)-3-(6-chloro-2-oxo-2,3-dihydro-1H-imidazo[4,5-c]pyridin-1-yl)cyclopentyl-1-d)carbamate (4.9 g, 15.7 mmol) and cesium carbonate (7.7 g, 23.6 mmol) in THF (40 mL) was added iodomethane-d₃ (6.8 g, 2.9 mL, 47.2 mmol). After stirring at r.t for 6 h, the reaction mixture was diluted with CH₂Cl₂ (100 mL), filtered through a Celite pad and washed with CH₂Cl₂ (3×50 mL). The filtrate was washed with aqueous saturated sodium bicarbonate solution, brine, dried over MgSO₄ and concentrated in vacuo. The crude material was purified by Biotage Isolera to afford the title compound. LCMS calculated for C₁₄H₁₄D₄ClN₄O₃(M+H)⁺: m/z=329.1; found: 329.1.

### Step 9. Methyl ((1R,3R)-3-(6-amino-3-(methyl-d₃)-2-oxo-2,3-dihydro-1H-imidazo[4,5-c]pyridin-1-yl)cyclopentyl-1-d)carbamate

A flask containing methyl ((1R,3R)-3-(6-chloro-3-(methyl-d₃)-2-oxo-2,3-dihydro-1H-imidazo[4,5-c]pyridin-1-yl)cyclopentyl-1-d)carbamate (4.0 g, 12.2 mmol), diphenylmethanimine (4.4 g, 24.3 mmol), tris(dibenzylideneacetone)-dipalladium(0) (1.11 g, 1.22 mmol), 4,5-bis(diphenylphosphino)-9,9-dimethylxanthene (1.41 g, 2.43 mmol) and cesium carbonate (11.9 g, 36.5 mmol) was evacuated and backfilled with nitrogen three times, followed by the addition of 1,4-dioxane (50.0 mL). The flask was heated at 85° C. for 16 h under nitrogen atmosphere. After cooling down to r.t, the reaction was diluted with DCM, filtered through Celite and concentrated in vacuo. The crude material was diluted with MeOH (30 mL) followed by the addition of hydroxylamine hydrochloride (2.4 g, 33.0 mmol) and potassium carbonate (4.5 g, 33 mmol). After stirring at at r.t for 1 h, the reaction mixture was filtered through Celite, concentrated in vacuo, purified by Biotage Isolera to give the desired product as white solid. LCMS calculated for C₁₄H₁₆D₄N₅O₃(M+H)⁺: m/z=310.2; found: 310.2.

### Alternative Synthesis of Intermediate A. Methyl ((1R,3R)-3-(6-amino-3-(methyl-d₃)-2-oxo-2,3-dihydro-1H-imidazo[4,5-c]pyridin-1-yl)cyclopentyl-1-d)carbamate

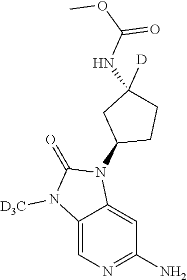 [SMILES: [2H]C([2H])([2H])n1c(=O)n([C@@H]2CC[C@@]([2H])(NC(=O)OC)C2)c2cc(N)ncc21]

### Step 1. tert-Butyl ((1R,3R)-3-((2-chloro-5-nitropyridin-4-yl)amino)cyclopentyl-1-d)carbamate

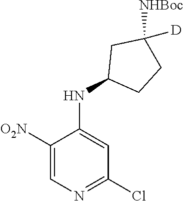 [SMILES: [2H][C@@]1(NC(=O)OC(C)(C)C)CC[C@@H](Nc2cc(Cl)ncc2[N+](=O)[O-])C1]

To a flask containing tert-butyl ((1R,3R)-3-aminocyclopentyl-1-d)carbamate hydrochloride (40.0 g, 168 mmol, PharmaBlock catalog #: PCS4754) and 2,4-dichloro-5-nitropyridine (32.5 g, 168 mmol) in acetonitrile (56.0 ml) was added DIPEA (64.6 mL, 370 mmol). The reaction mixture was stirred at 60° C. for 4 h. After cooling down to r.t., the reaction mixture was diluted with water (250 mL) and filtered. The filter cake was washed with water (100 mL) for 3 times and air dried. The obtained crude product was used in the next step without further purification. LCMS calculated for C₁₅H₂₁DClN₄O₄(M+H)⁺: m/z=358.1; found 358.1.

### Step 2. tert-Butyl ((1R,3R)-3-((5-amino-2-chloropyridin-4-yl)amino)cyclopentyl-1-d)carbamate

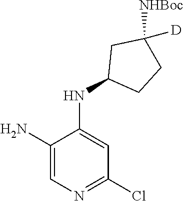 [SMILES: [2H][C@@]1(NC(=O)OC(C)(C)C)CC[C@@H](Nc2cc(Cl)ncc2N)C1]

A round-bottom flask containing zinc powder (22 g, 336 mmol) and ammonium chloride (18 g, 336 mmol) in MeOH (300 mL) was placed in an ice-water bath at 0° C. A solution of tert-butyl ((1R,3R)-3-((2-chloro-5-nitropyridin-4-yl)amino)cyclopentyl-1-d)carbamate (14.7 g, 41.2 mmol) in 120 mL (CH₂Cl₂:MeOH=5:1) was added via addition funnel to the zinc power suspension at the rate of 1 drop/sec with vigorous stirring. After stirring at r.t. for 3 h, the reaction mixture was filtered and washed with CH₂Cl₂ (100 mL) three times. The filtrate was collected and concentrated. The residue was re-dissolved in CH₂Cl₂ (300 mL), washed with water (50 mL), and extracted with CH₂Cl₂. The organic phase was washed with brine, dried over MgSO₄ and concentrated in vacuo. The obtained crude product was used in the next step without further purification. LCMS calculated for C₁₅H₂₃DClN₄O₂(M+H)⁺: m/z=328.2; found 328.2.

### Step 3. tert-Butyl ((1R,3R)-3-(6-chloro-2-oxo-2,3-dihydro-1H-imidazo[4,5-c]pyridin-1-yl)cyclopentyl-1-d)carbamate

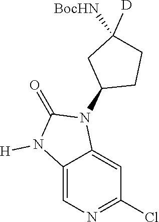 [SMILES: [2H][C@@]1(NC(=O)OC(C)(C)C)CC[C@@H](n2c(=O)[nH]c3cnc(Cl)cc32)C1]

To a flask containing tert-butyl ((1R,3R)-3-((5-amino-2-chloropyridin-4-yl)amino)cyclopentyl-1-d)carbamate (55.0 g, 168 mmol) in acetonitrile (420 mL) was added bis(2,5-dioxopyrrolidin-1-yl) carbonate (47.3 g, 185 mmol) in portions (over 20 times) during 30 min. After stirring at r.t. for 16 h, the reaction mixture was filtered to obtain the solid product as off-white solid. The filtrate was concentrated down to 80 mL in volume, quenched with aqueous saturated sodium carbonate solution and filtered. The solid was combined and washed with acetonitrile (80 mL) for 3 times and air dried. The obtained crude product was used in the next step without further purification. LCMS calculated for C₁₆H₂₁DClN₄O₃ (M+H)⁺: m/z=354.1; found: 354.1.

### Step 4. 1-((1R,3R)-3-Aminocyclopentyl-3-d)-6-chloro-3-(methyl-d₃)-1,3-dihydro-2H-imidazo[4,5-c]pyridin-2-one

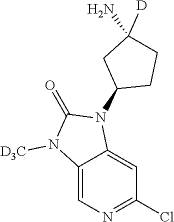 [SMILES: [2H]C([2H])([2H])n1c(=O)n([C@@H]2CC[C@@]([2H])(N)C2)c2cc(Cl)ncc21]

To a flask containing tert-butyl ((1R,3R)-3-(6-chloro-2-oxo-2,3-dihydro-1H-imidazo[4,5-c]pyridin-1-yl)cyclopentyl-1-d)carbamate (55.0 g, 155 mmol) and cesium carbonate (65.8 g, 202 mmol) in THF (390 mL) was added iodomethane-d₃ (45.1 g, 19.3 mL, 311 mmol). After stirring at r.t. for 4 h, the reaction mixture was diluted with CH₂Cl₂ (500 mL), filtered through a celite pad, and washed with CH₂Cl₂ (300 mL) for 3 times. The filtrate was concentrated in vacuo and was washed with aqueous saturated sodium bicarbonate solution. The organic phase was washed with brine, dried over MgSO₄ and concentrated in vacuo. The crude material was dissolved in 4 M HCl in dioxane (400 mL) and MeOH (50 mL). After stirring at 30° C. for 1 h, the reaction mixture was concentrated in vacuo. The obtained crude product as HCl salt of the title compound was used in the next step without further purification. LCMS calculated for C₁₂H₁₂D₄ClN₄O (M+H)⁺: m/z=271.1; found: 271.1.

### Step 5. Methyl ((1R,3R)-3-(6-chloro-3-(methyl-d₃)-2-oxo-2,3-dihydro-1H-imidazo[4,5-c]pyridin-1-yl)cyclopentyl-1-d)carbamate

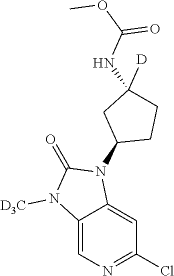 [SMILES: [2H]C([2H])([2H])n1c(=O)n([C@@H]2CC[C@@]([2H])(NC(=O)OC)C2)c2cc(Cl)ncc21]

To a flask containing 1-((1R,3R)-3-aminocyclopentyl-3-d)-6-chloro-3-(methyl-d₃)-1,3-dihydro-2H-imidazo[4,5-c]pyridin-2-one (53.0 g, 154 mmol) and DIPEA (89 mL, 512 mmol) in CH₂Cl₂ (400 mL) and MeOH (20 mL) at 0° C. was added methyl carbonochloridate (16.6 g, 13.6 mL, 176 mmol). After stirring at 0° C. for 5 min, the reaction mixture was quenched with MeOH and concentrated in vacuo. The obtained crude product was re-dissolved in CH₂Cl₂ (750 mL), was washed with brine, dried over MgSO₄ and concentrated in vacuo. The product was purified by washing with 20 mL (CH₂Cl₂:MeOH=20:1) for 3 times to give the desired product as white solid after air drying. LCMS calculated for C₁₄H₁₄D₄ClN₄O₃(M+H)⁺: m/z=329.1; found: 329.1.

### Step 6. Methyl ((1R,3R)-3-(6-amino-3-(methyl-d₃)-2-oxo-2,3-dihydro-1H-imidazo[4,5-c]pyridin-1-yl)cyclopentyl-1-d)carbamate

A flask containing methyl ((1R,3R)-3-(6-chloro-3-(methyl-d₃)-2-oxo-2,3-dihydro-1H-imidazo[4,5-c]pyridin-1-yl)cyclopentyl-1-d)carbamate (4.0 g, 12.2 mmol), diphenylmethanimine (4.4 g, 24.3 mmol), tris(dibenzylideneacetone)-dipalladium(0) (1.11 g, 1.22 mmol), 4,5-bis(diphenylphosphino)-9,9-dimethylxanthene (1.41 g, 2.43 mmol) and cesium carbonate (11.9 g, 36.5 mmol) was evacuated and backfilled with nitrogen three times, followed by the addition of 1,4-dioxane (50.0 mL). The flask was heated at 85° C. for 16 h under nitrogen atmosphere. After cooling down to r.t., the reaction was diluted with DCM, filtered through Celite and concentrated in vacuo. The crude material was diluted with MeOH (30 mL) followed by the addition of hydroxylamine hydrochloride (2.4 g, 33.0 mmol) and potassium carbonate (4.5 g, 33 mmol). After stirring at r.t for 1 h, the reaction mixture was filtered through Celite, concentrated in vacuo, and purified by Biotage Isolera to give the desired product as white solid. LCMS calculated for C₁₄H₁₆D₄N₅O₃(M+H)⁺: m/z=310.2; found: 310.2.

### Intermediate B. Methyl ((3R)-3-(6-amino-3-(methyl-d₃)-2-oxo-2,3-dihydro-1H-imidazo[4,5-c]pyridin-1-yl)cyclopentyl-1-d)carbamate

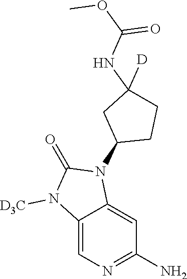 [SMILES: [2H]C1(NC(=O)OC)CC[C@@H](n2c(=O)n(C([2H])([2H])[2H])c3cnc(N)cc32)C1]

This compound was prepared according to the procedures described in Intermediate A, Step 1 to 9, with tert-butyl ((1R)-3-hydroxycyclopentyl-3-d)carbamate (Intermediate A, Step 1, mixture of diastereomers) replacing tert-butyl ((1R,3R)-3-hydroxycyclopentyl-3-d)carbamate in Step 2 to give the title compound as a mixture of two diastereomers. LCMS calculated for C₁₄H₁₆D₄N₅O₃(M+H)⁺: m/z=310.2; found: 310.2.

### Intermediate C. Methyl-d₃ ((1R,3R)-3-(6-amino-3-(methyl-d₃)-2-oxo-2,3-dihydro-1H-imidazo[4,5-c]pyridin-1-yl)cyclopentyl)carbamate

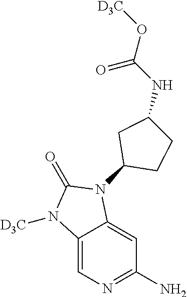 [SMILES: [2H]C([2H])([2H])OC(=O)N[C@@H]1CC[C@@H](n2c(=O)n(C([2H])([2H])[2H])c3cnc(N)cc32)C1]

### Step 1. 1-((1R,3R)-3-Aminocyclopentyl)-6-chloro-3-(methyl-d₃)-1,3-dihydro-2H-imidazo[4,5-c]pyridin-2-one

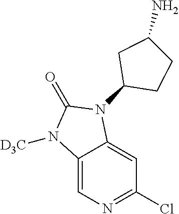 [SMILES: [2H]C([2H])([2H])n1c(=O)n([C@@H]2CC[C@@H](N)C2)c2cc(Cl)ncc21]

This compound was prepared according to the procedures described in the Alternative Synthesis of Intermediate A (Steps 1-4), with tert-butyl ((1R,3R)-3-aminocyclopentyl)carbamate replacing tert-butyl ((1R,3R)-3-aminocyclopentyl-1-d)carbamate hydrochloride. LCMS calculated for C₁₂H₁₃D₃ClN₄O (M+H)⁺: m/z=270.1; found: 270.1.

### Step 2. Methyl-d₃ ((1R,3R)-3-(6-chloro-3-(methyl-d₃)-2-oxo-2,3-dihydro-1H-imidazo[4,5-c]pyridin-1-yl)cyclopentyl)carbamate

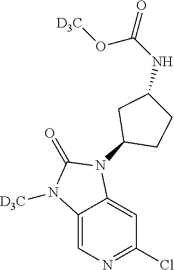 [SMILES: [2H]C([2H])([2H])OC(=O)N[C@@H]1CC[C@@H](n2c(=O)n(C([2H])([2H])[2H])c3cnc(Cl)cc32)C1]

This compound was prepared according to the procedures described in the Alternative Synthesis of Intermediate A (Step 5), with methyl-d₃ carbonochloridate and 1-((1R,3R)-3-aminocyclopentyl)-6-chloro-3-(methyl-d₃)-1,3-dihydro-2H-imidazo[4,5-c]pyridin-2-one replacing methyl carbonochloridate and 1-((1R,3R)-3-aminocyclopentyl-3-d)-6-chloro-3-(methyl-d₃)-1,3-dihydro-2H-imidazo[4,5-c]pyridin-2-one. LCMS calculated for C₁₄H₁₂D₆ClN₄O₃(M+H)⁺: m/z=331.1; found: 331.1.

### Step 3. Methyl-d₃ ((1R,3R)-3-(6-amino-3-(methyl-d₃)-2-oxo-2,3-dihydro-1H-imidazo[4,5-c]pyridin-1-yl)cyclopentyl)carbamate

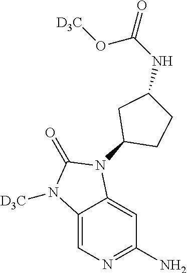 [SMILES: [2H]C([2H])([2H])OC(=O)N[C@@H]1CC[C@@H](n2c(=O)n(C([2H])([2H])[2H])c3cnc(N)cc32)C1]

A flask containing methyl-d₃ ((1R,3R)-3-(6-chloro-3-(methyl-d₃)-2-oxo-2,3-dihydro-1H-imidazo[4,5-c]pyridin-1-yl)cyclopentyl)carbamate (4.00 g, 12.09 mmol), diphenylmethanimine (4.06 ml, 24.18 mmol), tris(dibenzylideneacetone)dipalladium (1.11 g, 1.21 mmol), 9,9-dimethyl-4,5-bis(diphenylphosphino)xanthene (1.4 g, 2.42 mmol), and cesium carbonate (11.8 g, 36.3 mmol) was evacuated and backfilled with N₂ three times, followed by addition of dioxane (40 ml) and sparged with N₂ for 3 min. The reaction mixture was heated to 90° C. for 16 h. The reaction was then cooled to rt, diluted with DCM and filtered through celite. The crude was concentrated and treated with MeOH (40 mL), hydroxylamine hydrochloride (3.36 g, 48.4 mmol), and potassium carbonate (6.68 g, 48.4 mmol), then left to stir at rt for 2 h. The reaction was concentrated. The obtained crude was then dissolved in DCM, filtered and purified on Biotage isolera (0-20% MeOH in DCM). LCMS calculated for C₁₄H₁₄D₆N₅O₃ (M+H)⁺: m/z=312.1; found: 312.1.

### Example 1. Methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

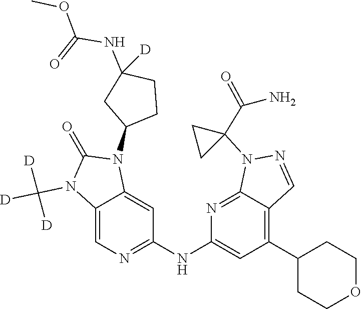 [SMILES: [2H]C1(NC(=O)OC)CC[C@@H](n2c(=O)n(C([2H])([2H])[2H])c3cnc(Nc4cc(C5CCOCC5)c5cnn(C6(C(N)=O)CC6)c5n4)cc32)C1]

### Step 1. tert-Butyl 2-(6-chloro-4-iodo-1H-pyrazolo[3,4-b]pyridin-1-yl)acetate

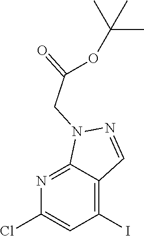 [SMILES: CC(C)(C)OC(=O)Cn1ncc2c(I)cc(Cl)nc21]

To a flask containing 6-chloro-4-iodo-1H-pyrazolo[3,4-b]pyridine (5.0 g, 17.9 mmol) and cesium carbonate (8.7 g, 26.8 mmol) in DMF (6.0 mL) was added tert-butyl 2-bromoacetate (2.9 mL, 19.7 mmol) After heating at 60° C. for 1 h, the reaction mixture was poured into an ice cold solution of saturated aqueous NH₄Cl and stirred for 10 min. The solid product from the reaction mixture was then filtered, followed by washing with water and hexanes to obtain the crude product. The crude material was purified by Biotage Isolera to afford the desired product. LCMS calculated for C₁₂H₁₄ClIN₃O₂(M+H)⁺: m/z=394.0; found 394.0.

### Step 2. tert-Butyl 1-(6-chloro-4-iodo-1H-pyrazolo[3,4-b]pyridin-1-yl)cyclopropane-1-carboxylate

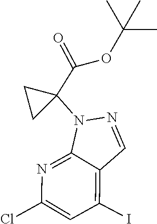 [SMILES: CC(C)(C)OC(=O)C1(n2ncc3c(I)cc(Cl)nc32)CC1]

To a flask containing tert-butyl 2-(6-chloro-4-iodo-1H-pyrazolo[3,4-b]pyridin-1-yl)acetate (4.5 g, 11.4 mmol) in THF (10.0 mL) at −78° C. was added LiHMDS (12.6 mL, 12.6 mmol, 1M in THF) in a dropwise manner, After stirring at −78° C. for 10 min., a solution of 1,3,2-dioxathiolane 2,2-dioxide (1.56 g, 12.6 mmol) in THF (0.5 mL) was added to the reaction mixture. After 5 min, the reaction mixture was warmed up to 0° C. After stirring at 0° C. for 1 h, it was cooled down to −78° C., followed by the addition of another portion of LiHMDS (12.6 mL, 12.6 mmol, 1M in THF) in a dropwise manner. The reaction mixture was allowed to warm up to rt by removing dry-ice bath. After stirring at r.t. for 16 h, the reaction was quenched with sat. aq. NH₄Cl (30 mL) and extraction with CH₂Cl₂. The organic layers were combined, dried over MgSO₄ and concentrated in vacuo. The crude material was purified by Biotage Isolera to afford the desired product. LCMS calculated for C₁₄H₁₆ClIN₃O₂ (M+H)⁺: m/z=420.0; found 420.0.

### Step 3. tert-Butyl 1-(6-chloro-4-(3,6-dihydro-2H-pyran-4-yl)-1H-pyrazolo[3,4-b]pyridin-1-yl)cyclopropane-1-carboxylate

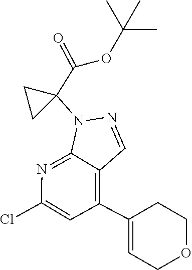 [SMILES: CC(C)(C)OC(=O)C1(n2ncc3c(C4=CCOCC4)cc(Cl)nc32)CC1]

To a vial containing tert-butyl 1-(6-chloro-4-iodo-1H-pyrazolo[3,4-b]pyridin-1-yl)cyclopropane-1-carboxylate (120 mg, 0.29 mmol), 2-(3,6-dihydro-2H-pyran-4-yl)-4,4,5,5-tetramethyl-1,3,2-dioxaborolane (66.1 mg, 0.32 mmol), tetrakis(triphenylphosphine)palladium(0) (33 mg, 0.029 mmol) and cesium carbonate (186 mg, 0.57 mmol) was added 1,4-dioxane (3.0 mL) and water (0.75 mL). The reaction mixture was flushed with nitrogen, then sealed and heated at 75° C. for 2 h. After cooling down to r.t., the reaction mixture was diluted with CH₂Cl₂, filtered through Celite and washed with CH₂Cl₂. The CH₂Cl₂ filtrates were combined, dried over MgSO₄ and concentrated in vacuo. The crude product was purified by Biotage Isolera to afford the desired compound. LCMS calculated for C₁₉H₂₃ClN₃O₃(M+H)⁺: m/z=376.1; found 376.1.

### Step 4. tert-Butyl 1-(4-(3,6-dihydro-2H-pyran-4-yl)-6-((1-((1R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclopropane-1-carboxylate

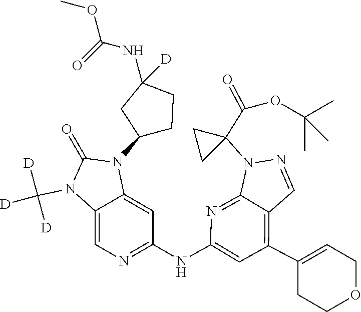 [SMILES: [2H]C1(NC(=O)OC)CC[C@@H](n2c(=O)n(C([2H])([2H])[2H])c3cnc(Nc4cc(C5=CCOCC5)c5cnn(C6(C(=O)OC(C)(C)C)CC6)c5n4)cc32)C1]

To a vial containing methyl ((3R)-3-(6-amino-3-(methyl-d₃)-2-oxo-2,3-dihydro-1H-imidazo[4,5-c]pyridin-1-yl)cyclopentyl-1-d)carbamate (80 mg, 0.26 mmol, Intermediate B), tert-butyl 1-(6-chloro-4-(3,6-dihydro-2H-pyran-4-yl)-1H-pyrazolo[3,4-b]pyridin-1-yl)cyclopropane-1-carboxylate (97 mg, 0.26 mmol), tris(dibenzylideneacetone)dipalladium(0) (24 mg, 0.026 mmol), 4,5-bis(diphenylphosphino)-9,9-dimethylxanthene (30 mg, 0.052 mmol) and cesium carbonate (253 mg, 0.78 mmol) was added 1,4-dioxane (5.0 mL). The reaction mixture was flushed with nitrogen, then sealed and heated at 105° C. for 16 h. After cooling down to r.t., the reaction mixture was diluted with CH₂Cl₂, filtered through Celite and washed with CH₂Cl₂. The CH₂Cl₂ filtrates were combined, dried over MgSO₄ and concentrated in vacuo. The crude product was purified by Biotage Isolera to afford the desired product. LCMS calculated for C₃₃H₃₇D₄N₈O₆(M+H)⁺: m/z=649.3; found 649.2.

### Step 5. 1-(6-((1-((1R)-3-((Methoxycarbonyl)amino)cyclopentyl-3-d)-3-(methyl-d₃)-2-oxo-2,3-dihydro-1H-imidazo[4,5-c]pyridin-6-yl)amino)-4-(tetrahydro-2H-pyran-4-yl)-1H-pyrazolo[3,4-b]pyridin-1-yl)cyclopropane-1-carboxylic acid

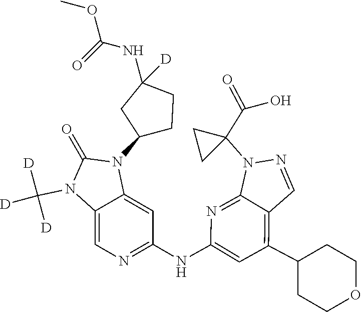 [SMILES: [2H]C1(NC(=O)OC)CC[C@@H](n2c(=O)n(C([2H])([2H])[2H])c3cnc(Nc4cc(C5CCOCC5)c5cnn(C6(C(=O)O)CC6)c5n4)cc32)C1]

To a vial containing tert-butyl 1-(4-(3,6-dihydro-2H-pyran-4-yl)-6-((1-((1R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclopropane-1-carboxylate (40 mg, 0.062 mmol) in MeOH (2.0 mL) was added Pd/C (6.6 mg, 10% w/w). The vial was purged with hydrogen gas for 5 min. and then stirred for 5 h under an atmosphere of hydrogen at 40° C. After cooling to room temperature, the reaction mixture was filtered through Celite and washed with CH₂Cl₂, followed by concentration of the filtrate in vacuo. The crude product was treated with 4 M HCl in 1,4-dioxane (2 mL). After heating at 60° C. for 30 min, the reaction mixture was concentrated in vacuo. The obtained crude product as the HCl salt of the title compound was used in the next step without further purification. LCMS calculated for C₂₉H₃₁D₄N₈O₆(M+H)⁺: m/z=595.3; found 595.2.

### Step 6. Methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a vial containing 1-(6-((1-((1R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(tetrahydro-2H-pyran-4-yl)-1H-pyrazolo[3,4-b]pyridin-1-yl)cyclopropane-1-carboxylic acid (80 mg, 0.135 mmol), ammonia (1.0 mL, 0.5 M in dioxane) and DIPEA (0.12 mL, 0.68 mmol) in DMF (3 mL) was added HATU (77 mg, 0.20 mmol). After stirring at r.t. for 30 min, the crude product was diluted with acetonitrile and purified by prep-LCMS as the first peak of two diastereoisomers (XBridge C18 column, eluting with a gradient of 14.0→32.0% acetonitrile in water containing 0.1% TFA, at flow rate of 60 mL/min over 12 min). The desired product was collected at a retention time of 9.1 min as a single diastereomer. LCMS calculated for C₂₉H₃₂D₄N₉O₅(M+H)⁺: m/z=594.3; found 594.2. ¹H NMR (500 MHz, DMSO) δ 10.96 (s, 1H), 8.32 (s, 1H), 8.27 (s, 1H), 7.80 (brs, 1H), 7.34 (s, 1H), 7.30 (s, 1H), 6.90 (s, 1H), 6.64 (s, 1H), 4.90-4.80 (m, 1H), 4.06-3.99 (m, 2H), 3.61-3.52 (m, 5H), 3.30-3.22 (m, 1H), 2.36 (dd, J=13.6, 7.6 Hz, 1H), 2.25-2.02 (m, 3H), 1.98-1.92 (m, 1H), 1.90-1.75 (m, 6H), 1.65 (dt, J=12.1, 7.9 Hz, 1H), 1.60-1.54 (m, 2H).

### Example 2. Methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

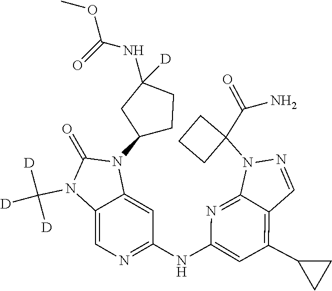 [SMILES: [2H]C1(NC(=O)OC)CC[C@@H](n2c(=O)n(C([2H])([2H])[2H])c3cnc(Nc4cc(C5CC5)c5cnn(C6(C(N)=O)CCC6)c5n4)cc32)C1]

### Step 1. Ethyl 1-(6-chloro-4-iodo-1H-pyrazolo[3,4-b]pyridin-1-yl)cyclobutane-1-carboxylate

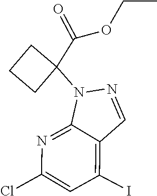 [SMILES: CCOC(=O)C1(n2ncc3c(I)cc(Cl)nc32)CCC1]

To a vial containing 2,6-dichloro-4-iodonicotinaldehyde (700 mg, 2.32 mmol) and ethyl 1-hydrazineylcyclobutane-1-carboxylate hydrochloride (470 mg, 2.43 mmol) in EtOH (3 mL) was added DIPEA (1.0 mL, 5.8 mmol). The reaction vial was sealed and heated 60° C. for 1 h. After cooling down to r.t., the reaction mixture was transferred to a microwave vial with the addition of DIPEA (1.0 mL, 5.8 mmol). The microwave vial was sealed and irradiated at 150° C. for 3 h. After cooling down to r.t., the reaction mixture was concentrated in vacuo. The obtained crude product was purified by Biotage Isolera to afford the desired product. LCMS calculated for C₁₃H₁₄ClIN₃O₂(M+H)⁺: m/z=406.0; found 406.0.

### Step 2. Ethyl 1-(6-chloro-4-cyclopropyl-1H-pyrazolo[3,4-b]pyridin-1-yl)cyclobutane-1-carboxylate

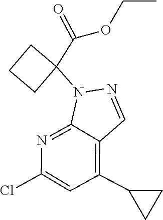 [SMILES: CCOC(=O)C1(n2ncc3c(C4CC4)cc(Cl)nc32)CCC1]

This compound was prepared according to the procedures described in Example 1, Step 3, with ethyl 1-(6-chloro-4-iodo-1H-pyrazolo[3,4-b]pyridin-1-yl)cyclobutane-1-carboxylate replacing tert-butyl 1-(6-chloro-4-iodo-1H-pyrazolo[3,4-b]pyridin-1-yl)cyclopropane-1-carboxylate and cyclopropyltrifluoro-14-borane, potassium salt replacing 2-(3,6-dihydro-2H-pyran-4-yl)-4,4,5,5-tetramethyl-1,3,2-dioxaborolane in Step 3. LCMS calculated for C₁₆H₁₉ClN₃O₂(M+H)⁺: m/z=320.1; found 320.1.

### Step 3. Ethyl 1-(4-cyclopropyl-6-((1-((1R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

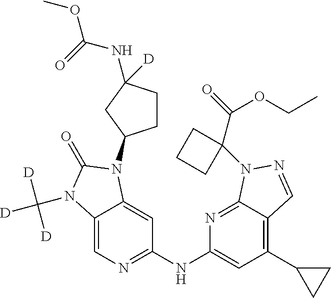 [SMILES: [2H]C1(NC(=O)OC)CC[C@@H](n2c(=O)n(C([2H])([2H])[2H])c3cnc(Nc4cc(C5CC5)c5cnn(C6(C(=O)OCC)CCC6)c5n4)cc32)C1]

This compound was prepared according to the procedures described in Example 1, Step 4, with ethyl 1-(6-chloro-4-cyclopropyl-1H-pyrazolo[3,4-b]pyridin-1-yl)cyclobutane-1-carboxylate replacing tert-butyl 1-(6-chloro-4-(3,6-dihydro-2H-pyran-4-yl)-1H-pyrazolo[3,4-b]pyridin-1-yl)cyclopropane-1-carboxylate in Step 4. LCMS calculated for C₃₀H₃₃D₄N₈O₅ (M+H)⁺: m/z=593.3; found 593.3.

### Step 4. Methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a vial containing ethyl 1-(4-cyclopropyl-6-((1-((1R)-3-((methoxycarbonyl)amino)-cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (38 mg, 0.065 mmol) in THF/MeOH/water (1.5 mL, v/v/v=1/1/1) was added LiOH (16 mg, 0.67 mmol). After heating at 50° C. for 15 min, the reaction mixture was cooled to r.t. and diluted with CH₂Cl₂ (3 mL). The pH of the mixture was adjusted to ˜4 with HCl aqueous solution, followed by the extraction with CH₂Cl₂ (3×5 mL). The organic layers were combined, dried over MgSO₄ and concentrated in vacuo. The obtained crude product was dissolved in DMF (1 mL), followed by the addition of ammonia (0.5 mL, 0.5 M in dioxane), DIPEA (0.12 mL, 0.68 mmol) and HATU (39 mg, 0.10 mmol). After stirring at r.t. for 30 min, the crude product was diluted with acetonitrile and purified by prep-LCMS as the first peak of two diastereoisomers (XBridge C18 column, eluting with a gradient of 17.8→35.8% acetonitrile in water containing 0.1% TFA, at flow rate of 60 mL/min over 12 min). The desired product was collected at a retention time of 10.8 min as a single diastereomer. LCMS calculated for C₂₈H₃₀D₄N₉O₄ (M+H)⁺: m/z=564.3; found 564.3.

### Example 3. Methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

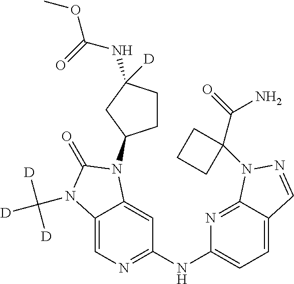 [SMILES: [2H]C([2H])([2H])n1c(=O)n([C@@H]2CC[C@@]([2H])(NC(=O)OC)C2)c2cc(Nc3ccc4cnn(C5(C(N)=O)CCC5)c4n3)ncc21]

### Step 1. Ethyl 1-(6-bromo-1H-pyrazolo[3,4-b]pyridin-1-yl)cyclobutane-1-carboxylate

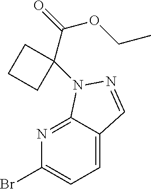 [SMILES: CCOC(=O)C1(n2ncc3ccc(Br)nc32)CCC1]

To a microwave vial containing 6-bromo-2-fluoronicotinaldehyde (2.0 g, 9.8 mmol) was added ethyl 1-hydrazineylcyclobutane-1-carboxylate hydrochloride (2.0 g, 10.3 mmol), NMP (9.8 mL) and DIPEA (8.6 mL, 49.0 mmol), then it was sealed and irradiated in the microwave at 180° C. for 2 h. After cooling down to r.t., the reaction mixture was diluted with EtOAc (50 mL) and water (20 mL). After extraction with EtOAc for 3 times, the organic layers were combined, washed with brine, dried over MgSO₄ and concentrated in vacuo. The crude material was purified by Biotage Isolera to afford the desired product. LCMS calculated for C₁₃H₁₅BrN₃O₂(M+H)⁺: m/z=324.0/326.0; found 324.0/326.0.

### Step 2. 1-(6-Bromo-1H-pyrazolo[3,4-b]pyridin-1-yl)cyclobutane-1-carboxylic acid

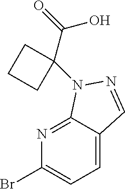 [SMILES: O=C(O)C1(n2ncc3ccc(Br)nc32)CCC1]

To a vial containing ethyl 1-(6-bromo-1H-pyrazolo[3,4-b]pyridin-1-yl)cyclobutane-1-carboxylate (2.0 g, 6.2 mmol) in THF (5 mL) was added lithium hydroxide, 2.0 M aq. (5 mL, 10.00 mmol). After heating at 60° C. for 16 h, the reaction mixture was cooled to r.t. and concentrated in vacuo to remove THF. The pH of the mixture was adjusted to ˜1 using 1 M HCl aqueous solution until a precipitate formed. The reaction mixture was filtered. The filter cake was washed with water and hexanes, followed by air drying. The obtained crude product was used in the next step without further purification. LCMS calculated for C₁₁H₁₁BrN₃O₂ (M+H)⁺: m/z=296.0/298.0; found 296.0/298.0.

### Step 3. 1-(6-Bromo-1H-pyrazolo[3,4-b]pyridin-1-yl)cyclobutane-1-carboxamide

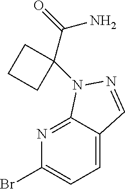 [SMILES: NC(=O)C1(n2ncc3ccc(Br)nc32)CCC1]

To a vial containing 1-(6-bromo-1H-pyrazolo[3,4-b]pyridin-1-yl)cyclobutane-1-carboxylic acid (3.41 g, 11.5 mmol) in DMF (10.0 mL) at 0° C. was added CDI (2.80 g, 17.3 mmol) as a suspension in 5 mL DMF. After heating at 60° C. for 1 h, the reaction mixture was cooled to r.t., followed by the addition of ammonium hydroxide (4.5 mL, 115 mmol). After heating at 60° C. for 2 h, the reaction mixture was diluted with CHCl₃/IPA (v/v=3/1) and 1 M HCl, followed by extraction with CHCl₃/IPA (v/v=3/1) 3 times. The organic phase was washed with brine, dried over MgSO₄ and concentrated in vacuo The crude material was purified by Biotage Isolera to afford the desired product. LCMS calculated for C₁₁H₁₂BrN₄O (M+H)⁺: m/z=295.0/297.0; found 295.0/297.0.

### Step 4. Methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (21 mg, 68 μmol, Intermediate A), 1-(6-bromo-1H-pyrazolo[3,4-b]pyridin-1-yl)cyclobutane-1-carboxamide (19 mg, 65 μmol), tris(dibenzylideneacetone)-dipalladium(0) (6 mg, 6.5 μmol), 4,5-bis(diphenylphosphino)-9,9-dimethylxanthene (7.5 mg, 13 μmol) and cesium carbonate (64 mg, 19.4 μmol) was evacuated and backfilled with nitrogen three times, followed by the addition of 1,4-dioxane (1.0 mL). The vial was sealed and heated to 120° C. for 2 h. After cooling to r.t., the reaction mixture was diluted with CH₂Cl₂, filtered through Celite and washed with CH₂Cl₂. The CH₂Cl₂ filtrates were combined, dried over MgSO₄ and concentrated in vacuo. The crude product was diluted with acetonitrile and purified by prep-LCMS (XBridge C18 column, eluting with a gradient of acetonitrile/water containing 0.1% TFA, at flow rate of 60 mL/min). LCMS calculated for C₂₅H₂₆D₄N₉O₄(M+H)⁺: m/z=524.3; found 524.3. ¹H NMR (500 MHz, DMSO) δ 11.54 (s, 1H), 8.28 (s, 1H), 8.25 (d, J=8.7 Hz, 1H), 8.18 (s, 1H), 7.50-7.42 (m, 2H), 7.38 (s, 1H), 7.32 (s, 1H), 6.99 (d, J=8.7 Hz, 1H), 5.04 (p, J=8.5 Hz, 1H), 3.55 (s, 3H), 3.00-2.81 (m, 4H), 2.34 (dd, J=13.6, 7.8 Hz, 1H), 2.26-2.12 (m, 2H), 2.11-2.03 (m, 1H), 2.02-1.93 (m, 3H), 1.66 (dt, J=12.2, 7.9 Hz, 1H).

### Example 4. Methyl ((1R,3R)-3-(6-((1-(1-carbamoylcyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate and Methyl ((1S,3R)-3-(6-((1-(1-carbamoylcyclobutyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

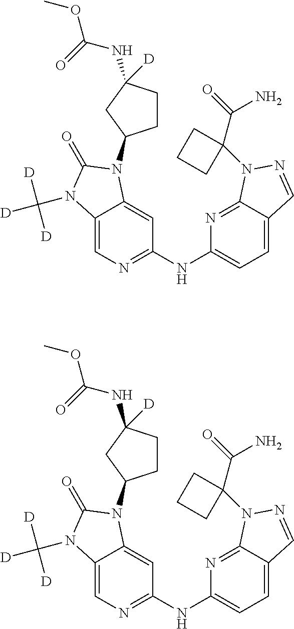 [SMILES: [2H]C([2H])([2H])n1c(=O)n([C@@H]2CC[C@@]([2H])(NC(=O)OC)C2)c2cc(Nc3ccc4cnn(C5(C(N)=O)CCC5)c4n3)ncc21 ; [2H]C([2H])([2H])n1c(=O)n([C@@H]2CC[C@]([2H])(NC(=O)OC)C2)c2cc(Nc3ccc4cnn(C5(C(N)=O)CCC5)c4n3)ncc21]

This compound was prepared according to the procedures described in Example 3, Step 4, with methyl ((3R)-3-(6-amino-3-(methyl-d₃)-2-oxo-2,3-dihydro-1H-imidazo[4,5-c]pyridin-1-yl)cyclopentyl-1-d)carbamate (Intermediate B) replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate (Intermediate A) in Step 4. The crude product was purified by prep-LCMS as the second peak of two diastereoisomers (XBridge C18 column, eluting with a gradient of 14.0→32.0% acetonitrile in water containing 0.1% TFA, at flow rate of 60 mL/min over 12 min). Retention time was 10.1 min for peak 1 and 10.7 min for peak 2. The desired product was collected at a retention time of 10.7 min as a single diastereomer. LCMS calculated for C₂₅H₂₆D₄N₉O₄(M+H)⁺: m/z=524.3; found 524.3. ¹H NMR (600 MHz, DMSO) δ 11.47 (s, 1H), 8.28 (s, 1H), 8.23 (d, J=8.6 Hz, 1H), 8.16 (s, 1H), 7.63 (brs, 1H), 7.41 (s, 1H), 7.39 (s, 1H), 7.18 (brs, 1H), 6.99 (d, J=8.7 Hz, 1H), 4.93 (p, J=8.7 Hz, 1H), 3.55 (s, 3H), 2.99-2.84 (m, 4H), 2.27 (dd, J=12.5, 8.2 Hz, 1H), 2.23-2.15 (m, 1H), 2.13-2.04 (m, 2H), 2.03-1.92 (m, 3H), 1.86-1.81 (m, 1H).

### Example 5. Methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

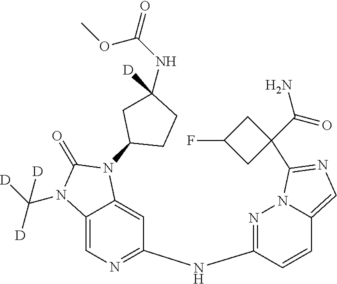 [SMILES: [2H]C([2H])([2H])n1c(=O)n([C@@H]2CC[C@@]([2H])(NC(=O)OC)C2)c2cc(Nc3ccc4cnc(C5(C(N)=O)CC(F)C5)n4n3)ncc21]

### Step 1. Isopropyl 1-(((6-chloropyridazin-3-yl)methyl)carbamoyl)-3-fluorocyclobutane-1-carboxylate

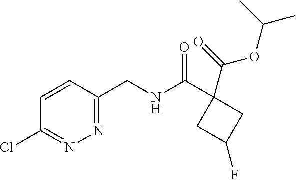 [SMILES: CC(C)OC(=O)C1(C(=O)NCc2ccc(Cl)nn2)CC(F)C1]

To a flask containing 3-fluoro-1-(isopropoxycarbonyl)cyclobutane-1-carboxylic acid (9.0 g, 43.9 mmol) in DCM (84 mL) was added N-ethyl-N-isopropylpropan-2-amine (22.3 mL, 125 mmol) followed by HATU (23.8 g, 62.7 mmol) and (6-chloropyridazin-3-yl)methanamine hydrochloride (7.6 g, 41.8 mmol). After stirring at r.t for 1 h, water (80 mL) was then added, and the mixture was extracted with DCM (3×40 mL). The organic phases were combined, dried over Na₂SO₄, filtered, concentrated in vacuo and the residue was purified by Biotage Isolera. LCMS calculated for C₁₄H₁₈ClFN₃O₃(M+H)⁺: m/z=330.1; found 330.1.

### Step 2. Isopropyl 1-(2-chloroimidazo[1,5-b]pyridazin-7-yl)-3-fluorocyclobutane-1-carboxylate

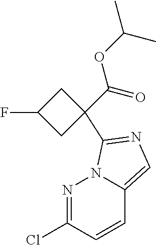 [SMILES: CC(C)OC(=O)C1(c2ncc3ccc(Cl)nn23)CC(F)C1]

A round-bottom flask containing a solution of isopropyl 1-(((6-chloropyridazin-3-yl)methyl)carbamoyl)-3-fluorocyclobutane-1-carboxylate (9.5 g, 28.8 mmol) in acetonitrile (450 mL) and phosphoryl trichloride (44.2 g, 288 mmol) was refluxed at 115° C. for 16 h. The reaction mixture was concentrated in vacuo and the residue was basified with sat. NaHCO₃ and extracted with EtOAc (3×50 mL). The organic phases were combined, dried over Na₂SO₄ and concentrated in vacuo. The obtained crude product was purified by Biotage Isolera, which was then subjected to SFC chiral purification to separate two diastereomers (ChiralTech IK 5 μm 30×250 mm column, eluting with a gradient of 15% IPA in CO₂, at flow rate of 90 mL/min). Retention time was 3.5 min for peak 1 and 4.9 min for peak 2. The titled product (peak 2) was obtained as a single diastereomer. LCMS calculated for C₁₄H₁₆ClFN₃O₂(M+H)⁺: m/z=312.1; found 312.1.

### Step 3. Isopropyl 3-fluoro-1-(2-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)imidazo[1,5-b]pyridazin-7-yl)cyclobutane-1-carboxylate

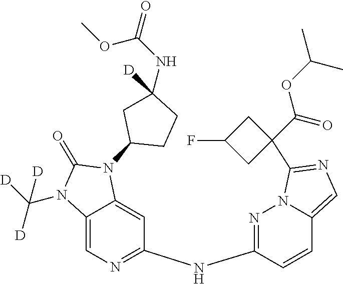 [SMILES: [2H]C([2H])([2H])n1c(=O)n([C@@H]2CC[C@@]([2H])(NC(=O)OC)C2)c2cc(Nc3ccc4cnc(C5(C(=O)OC(C)C)CC(F)C5)n4n3)ncc21]

A reaction vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (77 mg, 0.24 mmol), isopropyl 1-(2-chloroimidazo[1,5-b]pyridazin-7-yl)-3-fluorocyclobutane-1-carboxylate (Example 5—Step 2—Peak 2) (75 mg, 0.24 mmol), cesium carbonate (234 mg, 0.7 mmol), Pd₂(dba)₃ (44 mg, 0.05 mmol), and xantphos (55 mg, 0.1 mmol) was evacuated and backfilled with nitrogen. 1,4-Dioxane (2.5 mL) was added to the reaction mixture, which was then stirred at 110° C. for 1 h. The reaction mixture was cooled to r.t and diluted with EtOAc. The organic layer was subsequently washed with water and brine, dried over Na₂SO₄, filtered, and the solvents were evaporated in vacuo. The crude material was purified by Biotage Isolera. LCMS calculated for C₂₈H₃₀D₄FN₈O₅(M+H)⁺: m/z=585.3; found 585.3.

### Step 4. 3-Fluoro-1-(2-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)imidazo[1,5-b]pyridazin-7-yl)cyclobutane-1-carboxylic acid

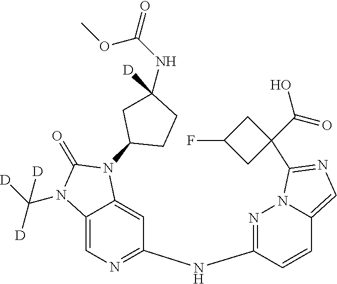 [SMILES: [2H]C([2H])([2H])n1c(=O)n([C@@H]2CC[C@@]([2H])(NC(=O)OC)C2)c2cc(Nc3ccc4cnc(C5(C(=O)O)CC(F)C5)n4n3)ncc21]

A solution of isopropyl 3-fluoro-1-(2-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)imidazo[1,5-b]pyridazin-7-yl)cyclobutane-1-carboxylate (122 mg, 0.21 mmol) and lithium hydroxide (49 mg, 2.1 mmol) in MeOH (1.5 mL), THF (1.5 mL), water (1.5 mL) was stirred at 65° C. for 5 h. The reaction was cooled to ambient temperature, acidified to pH 2-3 using 12 M HCl and diluted with EtOAc (6 mL). The layers were separated, and the aqueous layer was further extracted with EtOAc (3×6 mL). The organic layer was dried over Na₂SO₄, filtered, and concentrated in vacuo. The crude residue was carried forward without any further purification. LCMS calculated for C₂₅H₂₄D₄FN₈O₅ (M+H)⁺: m/z=543.2; found 543.2.

### Step 5. Methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

To a vial containing 3-fluoro-1-(2-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)imidazo[1,5-b]pyridazin-7-yl)cyclobutane-1-carboxylic acid (108 mg, 0.2 mmol), HATU (155 mg, 0.4 mmol) in DMF (2 mL) was added N-ethyl-N-isopropylpropan-2-amine (0.1 mL, 0.6 mmol) followed by 0.4 M ammonia in dioxane (1.5 mL, 0.6 mmol). The reaction mixture was stirred at r.t for 1 h. The reaction was quenched with water (10 mL) and extracted with EtOAc (3×10 mL). The combined organic layers were dried over Na₂SO₄, filtered, and concentrated in vacuo. The crude material was diluted with acetonitrile and purified by prep-LCMS (XBridge C18 column, eluting with a gradient of acetonitrile/water containing 0.1% TFA, at flow rate of 60 mL/min). The product was obtained as a single diastereomer. LCMS calculated for C₂₅H₂₅D₄FN₉O₄(M+H)⁺: m/z=542.3; found 542.3. ¹H NMR (400 MHz, DMSO) δ 10.92 (s, 1H), 8.26 (s, 1H), 8.15 (d, J=9.7 Hz, 1H), 7.85 (s, 1H), 7.56 (s, 1H), 7.48 (s, 1H), 7.39 (s, 1H), 7.29 (s, 1H), 6.91 (d, J=9.7 Hz, 1H), 5.43-5.21 (m, 1H), 5.14 (p, J=8.2 Hz, 1H), 3.35-3.23 (m, 2H), 3.02-2.80 (m, 2H), 2.51 (s, 3H), 2.37 (dd, J=13.5, 7.0 Hz, 1H), 2.23-2.14 (m, 2H), 2.11-2.04 (m, 1H), 1.99 (dd, J=13.5, 9.7 Hz, 1H), 1.81-1.54 (m, 1H).

### Example 6. Methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

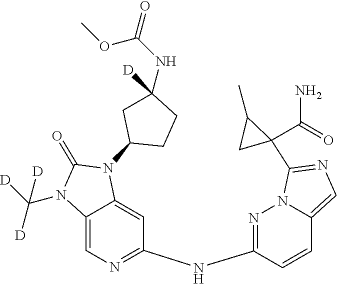 [SMILES: [2H]C([2H])([2H])n1c(=O)n([C@@H]2CC[C@@]([2H])(NC(=O)OC)C2)c2cc(Nc3ccc4cnc(C5(C(N)=O)CC5C)n4n3)ncc21]

### Step 1. Methyl 1-(2-chloroimidazo[1,5-b]pyridazin-7-yl)-2-methylcyclopropane-1-carboxylate

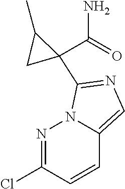 [SMILES: CC1CC1(C(N)=O)c1ncc2ccc(Cl)nn12]

The title compound was prepared according to the procedures described in Example 5, Step 1-2, with 1-(methoxycarbonyl)-2-methylcyclopropane-1-carboxylic acid replacing 3-fluoro-1-(isopropoxycarbonyl)cyclobutane-1-carboxylic acid in Step 1. The obtained crude product was purified by Biotage Isolera, which was then subjected to SFC chiral purification to separate two diastereomers (ChiralTech IK 5 um 21×250 mm column, eluting with a gradient of 8% MeOH in CO₂, at flow rate of 85 mL/min). Retention time was 7.6 min for peak 1 and 9.2 min for peak 2. The titled compound (peak 1) was obtained as a single diastereomer. LCMS calculated for C₁₂H₁₃ClN₃O₂(M+H)⁺: m/z=266.1; found: 266.1.

### Step 2. Methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 5, Step 3-5, with methyl 1-(2-chloroimidazo[1,5-b]pyridazin-7-yl)-2-methylcyclopropane-1-carboxylate (peak 1) replacing isopropyl 1-(2-chloroimidazo[1,5-b]pyridazin-7-yl)-3-fluorocyclobutane-1-carboxylate in Step 3. The desired product was obtained as a single diastereomer. LCMS calculated for C₂₅H₂₆D₄N₉O₄(M+H)⁺: m/z=524.3; found: 524.3.

### Example 7. Methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

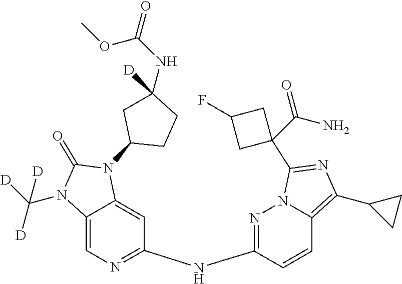 [SMILES: [2H]C([2H])([2H])n1c(=O)n([C@@H]2CC[C@@]([2H])(NC(=O)OC)C2)c2cc(Nc3ccc4c(C5CC5)nc(C5(C(N)=O)CC(F)C5)n4n3)ncc21]

### Step 1. Isopropyl 1-(2-chloro-5-iodoimidazo[1,5-b]pyridazin-7-yl)-3-fluorocyclobutane-1-carboxylate

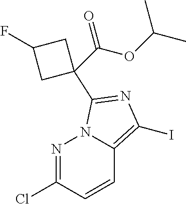 [SMILES: CC(C)OC(=O)C1(c2nc(I)c3ccc(Cl)nn23)CC(F)C1]

To a 40 mL vial containing isopropyl 1-(2-chloroimidazo[1,5-b]pyridazin-7-yl)-3-fluorocyclobutane-1-carboxylate (Example 5—Step 2—Peak 2) (0.62 g, 2 mmol) in DCM (5 mL) and DMF (5 mL) was added 1-iodopyrrolidine-2,5-dione (0.9 g, 4 mmol) at r.t. After stirring at 80° C. for 1 h, the reaction mixture was cooled down, quenched with water, and extracted with EtOAc. The organic layers were combined, dried over Na₂SO₄, filtered, and the solvents were evaporated in vacuo. The crude material was purified by Biotage Isolera. LCMS calculated for C₁₄H₁₅ClFIN₃O₂(M+H)⁺: m/z=438.1; found 438.1.

### Step 2. Isopropyl 1-(2-((2,4-dimethoxybenzyl)amino)-5-iodoimidazo[1,5-b]pyridazin-7-yl)-3-fluorocyclobutane-1-carboxylate

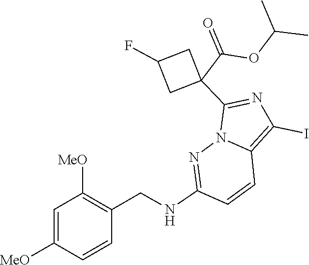 [SMILES: COc1ccc(CNc2ccc3c(I)nc(C4(C(=O)OC(C)C)CC(F)C4)n3n2)c(OC)c1]

To a vial containing isopropyl 1-(2-chloro-5-iodoimidazo[1,5-b]pyridazin-7-yl)-3-fluorocyclobutane-1-carboxylate (190 mg, 0.43 mmol) and (2,4-dimethoxyphenyl)methanamine (132 μL, 0.87 mmol) in DMSO (2 mL) was added N-ethyl-N-isopropylpropan-2-amine (232 μL, 1.3 mmol), which was then stirred at 110° C. for 2 h. The reaction mixture was cooled to r.t and water was added. The solid was then filtered and washed with water. The crude material was used in the next step without further purification. LCMS calculated for C₂₃H₂₇FIN₄O₄(M+H)⁺: m/z=569.1; found 569.1.

### Step 3. Isopropyl 1-(2-amino-5-cyclopropylimidazo[1,5-b]pyridazin-7-yl)-3-fluorocyclobutane-1-carboxylate

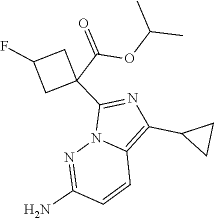 [SMILES: CC(C)OC(=O)C1(c2nc(C3CC3)c3ccc(N)nn23)CC(F)C1]

To a vial containing isopropyl 1-(2-((2,4-dimethoxybenzyl)amino)-5-iodoimidazo[1,5-b]pyridazin-7-yl)-3-fluorocyclobutane-1-carboxylate (120 mg, 0.21 mmol), Pd₂(dba)₃ (19 mg, 0.021 mmol), Qphos (30 mg, 0.042 mmol) in THF (3 mL) was added 0.5 M cyclopropylzinc(II) bromide in THF (1.6 mL, 0.63 mmol) under N₂ atmosphere. The reaction was stirred at 75° C. for 30 min. The solution was cooled down, diluted with water, and extracted with EtOAc. The organic layer was dried over Na₂SO₄, filtered, and the solvents were evaporated in vacuo. The crude material was then dissolved in TFA (4 mL) and stirred at 80° C. for 5 min. The reaction mixture was cooled to r.t, concentrated to remove volatiles, diluted with EtOAc and neutralized with sat. NaHCO₃. The organic layer was subsequently washed with water and brine, dried over Na₂SO₄, filtered, and the solvents were evaporated in vacuo. The crude material was purified by Biotage Isolera. LCMS calculated for C₁₇H₂₂FN₄O₂(M+H)⁺: m/z=333.2; found 333.2.

### Step 4. Methyl ((1R,3R)-3-(6-((7-(1-carbamoyl-3-fluorocyclobutyl)-5-cyclopropylimidazo[1,5-b]pyridazin-2-yl)amino)-3-(methyl-d3)-2-oxo-2,3-dihydro-1H-imidazo[4,5-c]pyridin-1-yl)cyclopentyl-1-d)carbamate

The title compound was prepared according to the procedures described in Example 5, Step 3-5, with isopropyl 1-(2-amino-5-cyclopropylimidazo[1,5-b]pyridazin-7-yl)-3-fluorocyclobutane-1-carboxylate replacing isopropyl 1-(2-chloroimidazo[1,5-b]pyridazin-7-yl)-3-fluorocyclobutane-1-carboxylate, and methyl ((1R,3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 3. The desired product was obtained as a single diastereomer. LCMS calculated for C₂₈H₂₉D₄FN₉O₄(M+H)⁺: m/z=582.3; found 582.3.

### Example 8. Methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

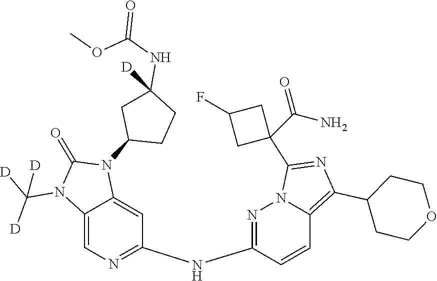 [SMILES: [2H]C([2H])([2H])n1c(=O)n([C@@H]2CC[C@@]([2H])(NC(=O)OC)C2)c2cc(Nc3ccc4c(C5CCOCC5)nc(C5(C(N)=O)CC(F)C5)n4n3)ncc21]

### Step 1. Isopropyl 1-(2-amino-5-(tetrahydro-2H-pyran-4-yl)imidazo[1,5-b]pyridazin-7-yl)-3-fluorocyclobutane-1-carboxylate

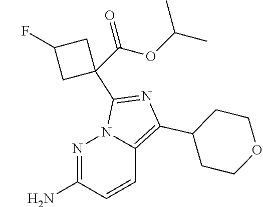

The title compound was prepared according to the procedures described in Example 7, Step 3, with (tetrahydro-2H-pyran-4-yl)zinc(II) bromide replacing cyclopropylzinc(II) bromide. LCMS calculated for C₁₉H₂₆FN₄O₃(M+H)⁺: m/z=377.2; found 377.2.

### Step 2. Methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 5, Step 3-5, with isopropyl 1-(2-amino-5-(tetrahydro-2H-pyran-4-yl)imidazo[1,5-b]pyridazin-7-yl)-3-fluorocyclobutane-1-carboxylate replacing isopropyl 1-(2-chloroimidazo[1,5-b]pyridazin-7-yl)-3-fluorocyclobutane-1-carboxylate, and methyl ((1R,3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 3. The desired product was obtained as a single diastereomer. LCMS calculated for C₃₀H₃₃D₄FN₉O₅(M+H)⁺: m/z=626.3; found 626.3.

### Example 9. Methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

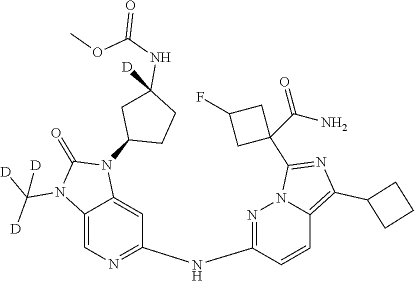 [SMILES: [2H]C([2H])([2H])n1c(=O)n([C@@H]2CC[C@@]([2H])(NC(=O)OC)C2)c2cc(Nc3ccc4c(C5CCC5)nc(C5(C(N)=O)CC(F)C5)n4n3)ncc21]

### Step 1. Isopropyl 1-(2-amino-5-cyclobutylimidazo[1,5-b]pyridazin-7-yl)-3-fluorocyclobutane-1-carboxylate

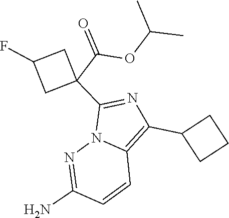 [SMILES: CC(C)OC(=O)C1(c2nc(C3CCC3)c3ccc(N)nn23)CC(F)C1]

The title compound was prepared according to the procedures described in Example 7, Step 3, with cyclobutylzinc(II) bromide replacing cyclopropylzinc(II) bromide. LCMS calculated for C₁₈H₂₄FN₄O₂(M+H)⁺: m/z=347.2; found 347.2.

### Step 2. Methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 5, Step 3-5, with isopropyl 1-(2-amino-5-cyclobutylimidazo[1,5-b]pyridazin-7-yl)-3-fluorocyclobutane-1-carboxylate replacing isopropyl 1-(2-chloroimidazo[1,5-b]pyridazin-7-yl)-3-fluorocyclobutane-1-carboxylate, and methyl ((1R,3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 3. The desired product was obtained as a single diastereomer. LCMS calculated for C₂₉H₃₁D₄FN₉O₄(M+H)⁺: m/z=596.3; found 596.3.

### Example 10. Methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate

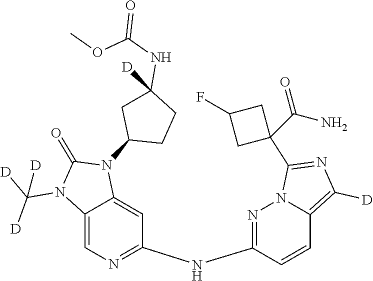 [SMILES: [2H]c1nc(C2(C(N)=O)CC(F)C2)n2nc(Nc3cc4c(cn3)n(C([2H])([2H])[2H])c(=O)n4[C@@H]3CC[C@@]([2H])(NC(=O)OC)C3)ccc12]

### Step 1. Isopropyl 1-(2-aminoimidazo[1,5-b]pyridazin-7-yl-5-d)-3-fluorocyclobutane-1-carboxylate

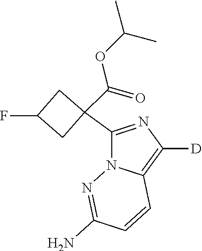 [SMILES: [2H]c1nc(C2(C(=O)OC(C)C)CC(F)C2)n2nc(N)ccc12]

A suspension of isopropyl 1-(2-((2,4-dimethoxybenzyl)amino)-5-iodoimidazo[1,5-b]pyridazin-7-yl)-3-fluorocyclobutane-1-carboxylate (Example 7—Step 2) (50 mg, 0.09 mmol) and Pd on carbon (10%, 11 mg, 0.009 mmol) in MeOH (5 ml) was stirred at 60° C. under a balloon of D₂ overnight. The reaction mixture was then filtered and concentrated in vacuo. The crude material was then dissolved in TFA (2 mL) and stirred at 60° C. for 5 min. The reaction mixture was cooled to r.t, concentrated to remove volatiles, diluted with EtOAc and neutralized with sat. NaHCO₃. The organic layer was subsequently washed with water and brine, dried over Na₂SO₄, filtered, and the solvents were evaporated in vacuo. The crude material was purified by Biotage Isolera. LCMS calculated for C₁₄H₁₇DFN₄O₂(M+H)⁺: m/z=294.2; found 294.2.

### Step 2. Methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 5, Step 3-5, with isopropyl 1-(2-aminoimidazo[1,5-b]pyridazin-7-yl-5-d)-3-fluorocyclobutane-1-carboxylate replacing isopropyl 1-(2-chloroimidazo[1,5-b]pyridazin-7-yl)-3-fluorocyclobutane-1-carboxylate, and 6-chloro-1-isopropyl-3-(methyl-d₃)-1,3-dihydro-2H-imidazo[4,5-c]pyridin-2-one replacing 6-amino-1-isopropyl-3-(methyl-d₃)-1,3-dihydro-2H-imidazo[4,5-c]pyridin-2-one in Step 3. The desired product was obtained as a single diastereomer. LCMS calculated for C₂₅H₂₄D₅FN₉O₄(M+H)⁺: m/z=543.3; found: 543.3.

### Example 11. Methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

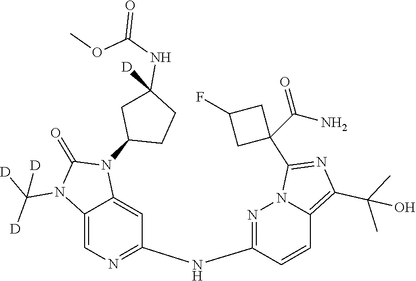 [SMILES: [2H]C([2H])([2H])n1c(=O)n([C@@H]2CC[C@@]([2H])(NC(=O)OC)C2)c2cc(Nc3ccc4c(C(C)(C)O)nc(C5(C(N)=O)CC(F)C5)n4n3)ncc21]

### Step 1. Isopropyl 1-(2-chloro-5-formylimidazo[1,5-b]pyridazin-7-yl)-3-fluorocyclobutane-1-carboxylate

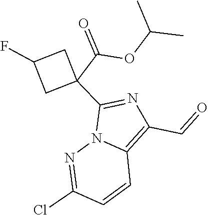 [SMILES: CC(C)OC(=O)C1(c2nc(C=O)c3ccc(Cl)nn23)CC(F)C1]

To a vial containing DMF (2 mL) was added dropwise phosphoryl trichloride (1.5 g, 10 mmol) at r.t. The mixture was stirred at r.t for 15 min. To this solution was added isopropyl 1-(2-chloroimidazo[1,5-b]pyridazin-7-yl)-3-fluorocyclobutane-1-carboxylate (Example 5—Step 2—Peak 2) (0.31 g, 1 mmol) and the mixture was stirred at r.t for 5 min before heating to 80° C. for 2 h. The reaction was allowed to cool down, poured into ice water, neutralized with sat. NaHCO₃ solution and extracted with ethyl acetate (3×10 mL). The combined organic layers were subsequently washed with brine, dried over Na₂SO₄, filtered, and the solvents were evaporated in vacuo. The crude material was purified by Biotage Isolera. LCMS calculated for C₁₅H₁₆ClFN₃O₃(M+H)⁺: m/z=340.1; found 340.1.

### Step 2. Methyl ((1R,3R)-3-(6-((7-(1-carbamoyl-3-fluorocyclobutyl)-5-formylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

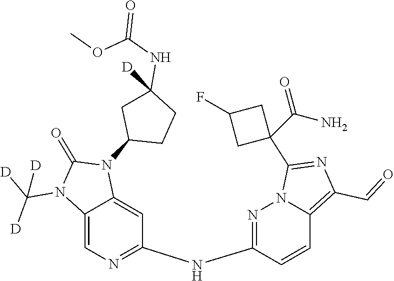 [SMILES: [2H]C([2H])([2H])n1c(=O)n([C@@H]2CC[C@@]([2H])(NC(=O)OC)C2)c2cc(Nc3ccc4c(C=O)nc(C5(C(N)=O)CC(F)C5)n4n3)ncc21]

The title compound was prepared according to the procedures described in Example 5, Step 3-5, with isopropyl 1-(2-chloro-5-formylimidazo[1,5-b]pyridazin-7-yl)-3-fluorocyclobutane-1-carboxylate replacing isopropyl 1-(2-chloroimidazo[1,5-b]pyridazin-7-yl)-3-fluorocyclobutane-1-carboxylate in Step 3. The crude material was purified by Biotage Isolera. LCMS calculated for C₂₆H₂₅D₄FN₉O₅(M+H)⁺: m/z=570.3; found: 570.3.

### Step 3. Methyl ((1R,3R)-3-(6-((7-(1-carbamoyl-3-fluorocyclobutyl)-5-(1-hydroxyethyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

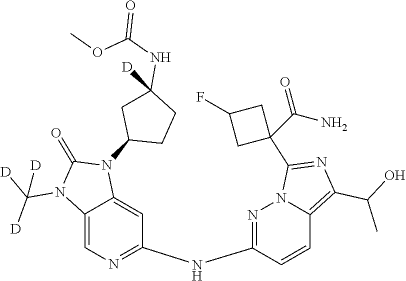 [SMILES: [2H]C([2H])([2H])n1c(=O)n([C@@H]2CC[C@@]([2H])(NC(=O)OC)C2)c2cc(Nc3ccc4c(C(C)O)nc(C5(C(N)=O)CC(F)C5)n4n3)ncc21]

To a vial containing methyl ((1R,3R)-3-(6-((7-(1-carbamoyl-3-fluorocyclobutyl)-5-formylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate (28 mg, 0.05 mmol) in THF (0.5 mL) was added 3 M methylmagnesium bromide in Et₂O (89 μL, 0.27 mmol) at r.t. After stirring for 5 min, the reaction was quenched with sat. NH₄Cl, extracted with EtOAc (3×5 mL). The combined organic layers were subsequently washed with brine, dried over Na₂SO₄, filtered, and the solvents were evaporated in vacuo. The crude material was used in the next step without further purification. LCMS calculated for C₂₇H₂₉D₄FN₉O₅(M+H)⁺: m/z=586.4; found 586.4.

### Step 4. Methyl ((1R,3R)-3-(6-((5-acetyl-7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

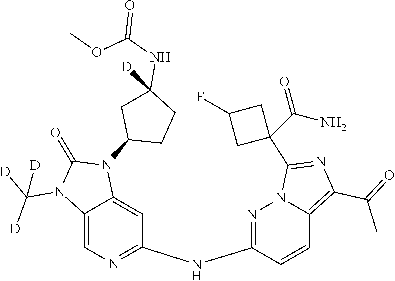 [SMILES: [2H]C([2H])([2H])n1c(=O)n([C@@H]2CC[C@@]([2H])(NC(=O)OC)C2)c2cc(Nc3ccc4c(C(C)=O)nc(C5(C(N)=O)CC(F)C5)n4n3)ncc21]

To a vial containing methyl ((1R,3R)-3-(6-((7-(1-carbamoyl-3-fluorocyclobutyl)-5-(1-hydroxyethyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate (60 mg, 0.1 mmol) in DCM (1 mL) was added 0.3 M of Dess-Martin periodinane in DCM (0.7 mL, 0.2 mmol) at r.t. After stirring for 30 min, the reaction was quenched with sat. NaHCO₃, extracted with DCM (3×5 mL). The combined organic layer was subsequently washed with brine, dried over Na₂SO₄, filtered, and the solvents were evaporated in vacuo. The crude material used in the next step without further purification. LCMS calculated for C₂₇H₂₇D₄FN₉O₅(M+H)⁺: m/z=584.3; found 584.3.

### Step 5. Methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

To a vial containing methyl ((1R,3R)-3-(6-((5-acetyl-7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate (29 mg, 0.05 mmol) in THF (0.5 mL) was added 3 M methylmagnesium bromide in Et₂O (89 μL, 0.27 mmol) at r.t. After stirring for 5 min, the reaction was quenched with sat. NH₄Cl, extracted with EtOAc (3×5 mL). The combined organic layers were subsequently washed with brine, dried over Na₂SO₄, filtered, and the solvents were evaporated in vacuo. The crude material was diluted with acetonitrile and purified by prep-LCMS (XBridge C18 column, eluting with a gradient of acetonitrile/water containing 0.1% TFA, at flow rate of 60 mL/min). The desired product was obtained as a single diastereomer. LCMS calculated for C₂₈H₃₁D₄FN₉O₅(M+H)⁺: m/z=600.2; found 600.2.

### Example 12. Methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1,2,2,5,5-d₃)carbamate

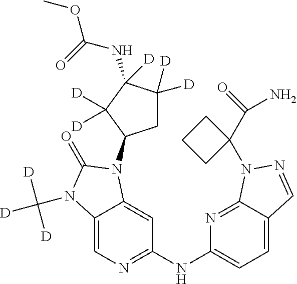 [SMILES: [2H]C([2H])([2H])n1c(=O)n([C@@H]2CC([2H])([2H])[C@@]([2H])(NC(=O)OC)C2([2H])[2H])c2cc(Nc3ccc4cnn(C5(C(N)=O)CCC5)c4n3)ncc21]

### Step 1. tert-Butyl (R)-(3-oxocyclopentyl-2,2,4,4-d₄)carbamate

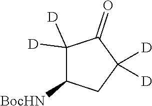 [SMILES: [2H]C1([2H])C[C@@H](NC(=O)OC(C)(C)C)C([2H])([2H])C1=O]

To a vial containing tert-butyl (R)-(3-oxocyclopentyl)carbamate (3.0 g, 15.1 mmol) in methanol-d₄ (10.0 mL) was added cesium carbonate (5 mg). After stirring at r.t. for 24 h, the reaction mixture was concentrated in vacuo. This process was repeated for two more times. After the third cycle, the reaction mixture was concentrated in vacuo. The crude product was used directly in the next step without further purification. ¹H NMR (400 MHz, CDCl₃) δ 4.22 (s, 1H), 2.35 (dd, J=13.1, 6.1 Hz, 1H), 1.85 (dd, J=13.1, 7.5 Hz, 1H), 1.47 (s, 9H).

### Step 2. Methyl ((1R,3R)-3-(6-amino-3-(methyl-d₃)-2-oxo-2,3-dihydro-1H-imidazo[4,5-c]pyridin-1-yl)cyclopentyl-1,2,2,5,5-d₅)carbamate

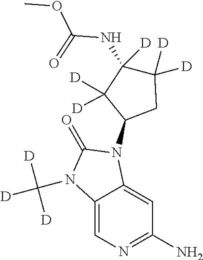 [SMILES: [2H]C([2H])([2H])n1c(=O)n([C@@H]2CC([2H])([2H])[C@@]([2H])(NC(=O)OC)C2([2H])[2H])c2cc(N)ncc21]

This compound was prepared according to the procedures described in Intermediate A, Step 1 to 9, with tert-butyl (R)-(3-oxocyclopentyl-2,2,4,4-d₄)carbamate replacing tert-butyl (R)-(3-oxocyclopentyl)carbamate in Step 1. LCMS calculated for C₁₄H₁₂D₈N₅O₃(M+H)⁺: m/z=314.2; found: 314.2.

### Example 13. Methyl ((1R,3R)-3-(6-((1-(1-carbamoylcyclobutyl)-4-(trans-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

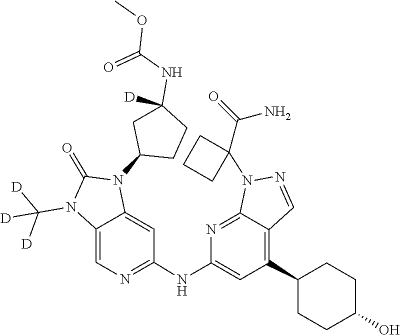 [SMILES: [2H]C([2H])([2H])n1c(=O)n([C@@H]2CC[C@@]([2H])(NC(=O)OC)C2)c2cc(Nc3cc([C@H]4CC[C@H](O)CC4)c4cnn(C5(C(N)=O)CCC5)c4n3)ncc21]

### Step 1. Ethyl 1-(6-chloro-4-(1,4-dioxaspiro[4.5]dec-7-en-8-yl)-1H-pyrazolo[3,4-b]pyridin-1-yl)cyclobutane-1-carboxylate

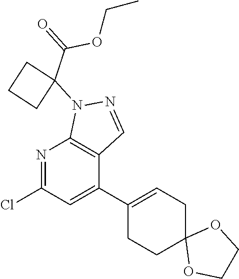 [SMILES: CCOC(=O)C1(n2ncc3c(C4=CCC5(CC4)OCCO5)cc(Cl)nc32)CCC1]

The mixture of ethyl 1-(6-chloro-4-iodo-1H-pyrazolo[3,4-b]pyridin-1-yl)cyclobutane-1-carboxylate (80 mg, 0.197 mmol), 4,4,5,5-tetramethyl-2-(1,4-dioxaspiro[4.5]dec-7-en-8-yl)-1,3,2-dioxaborolane (58 mg, 0.217 mmol), potassium phosphate tribasic (84 mg, 0.394 mmol) and (1,1′-bis(diphenylphosphino)ferrocene)-dichloropalladium(II) (24 mg, 0.030 mmol) in dioxane (0.4 ml) and water (0.1 ml) was evacuated and backfilled with nitrogen. The reaction was stirred at 60° C. for 2 hrs. The reaction was then cooled to r.t. and concentrated. The residue was purified by column chromatography eluted with EtOAc/hexane (0-50%) to obtain the desired compound. LCMS calculated for C₂₁H₂₅ClN₃O₄(M+H)⁺: m/z=418.2; found: 418.2.

### Step 2. Ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(1,4-dioxaspiro[4.5]dec-7-en-8-yl)-1H-pyrazolo[3,4-b]pyridin-1-yl)cyclobutane-1-carboxylate

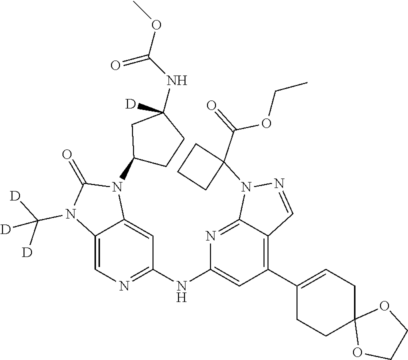 [SMILES: [2H]C([2H])([2H])n1c(=O)n([C@@H]2CC[C@@]([2H])(NC(=O)OC)C2)c2cc(Nc3cc(C4=CCC5(CC4)OCCO5)c4cnn(C5(C(=O)OCC)CCC5)c4n3)ncc21]

To a vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (74.0 mg, 0.167 mmol), Cs₂CO₃ (109 mg, 0.335 mmol), Pd₂(dba)₃ (30.7 mg, 0.033 mmol), xantphos (38.8 mg, 0.067 mmol) and ethyl 1-(6-chloro-4-(1,4-dioxaspiro[4.5]dec-7-en-8-yl)-1H-pyrazolo[3,4-b]pyridin-1-yl)cyclobutane-1-carboxylate (70.0 mg, 0.167 mmol) was added 1,4-dioxane (0.60 ml). The reaction was heated at 100° C. under N₂ overnight. The reaction was then cooled to r.t., diluted with DCM and filtered over celite. The solvent was removed in vacuo and the crude was purified by column chromatography (MeOH/DCM, 0-30%) to obtain the desired product. LCMS calculated for C₃₅H₃₉D₄N₈O₇(M+H)⁺: m/z=691.3; found: 691.5.

### Step 3. Ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(1,4-dioxaspiro[4.5]decan-8-yl)-1H-pyrazolo[3,4-b]pyridin-1-yl)cyclobutane-1-carboxylate

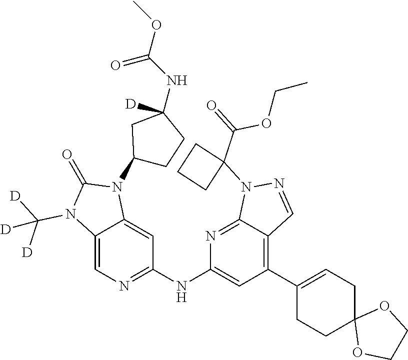 [SMILES: [2H]C([2H])([2H])n1c(=O)n([C@@H]2CC[C@@]([2H])(NC(=O)OC)C2)c2cc(Nc3cc(C4=CCC5(CC4)OCCO5)c4cnn(C5(C(=O)OCC)CCC5)c4n3)ncc21]

To a vial containing ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(1,4-dioxaspiro[4.5]dec-7-en-8-yl)-1H-pyrazolo[3,4-b]pyridin-1-yl)cyclobutane-1-carboxylate (72 mg, 0.104 mmol) and palladium hydroxide on carbon (20 wt. %, 14.6 mg, 0.021 mmol) was added MeOH (5 mL). The mixture was stirred under H₂ balloon at 60° C. for 2 hrs. The reaction mixture was then cooled to r.t., filtered, and concentrated to obtain the desired product. LCMS calculated for C₃₅H₄₁D₄N₈O₇(M+H)⁺: m/z=693.4; found: 693.5.

### Step 4. 1-(6-((1-((1R,3R)-3-((Methoxycarbonyl)amino)cyclopentyl-3-d)-3-(methyl-d₃)-2-oxo-2,3-dihydro-1H-imidazo[4,5-c]pyridin-6-yl)amino)-4-(4-oxocyclohexyl)-1H-pyrazolo[3,4-b]pyridin-1-yl)cyclobutane-1-carboxylic acid

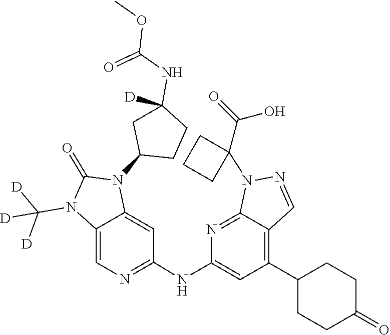 [SMILES: [2H]C([2H])([2H])n1c(=O)n([C@@H]2CC[C@@]([2H])(NC(=O)OC)C2)c2cc(Nc3cc(C4CCC(=O)CC4)c4cnn(C5(C(=O)O)CCC5)c4n3)ncc21]

To a solution of ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(1,4-dioxaspiro[4.5]decan-8-yl)-1H-pyrazolo[3,4-b]pyridin-1-yl)cyclobutane-1-carboxylate (64 mg, 0.092 mmol) in THF (1 ml), MeOH (1.000 ml) and water (0.2 ml) was added lithium hydroxide (11 mg, 0.462 mmol). The mixture was stirred at 60° C. for 30 min. The reaction was cooled to r.t. and quenched with HCl (0.077 ml, 0.924 mmol) and the resulting mixture was concentrated. The crude residue was dissolved in MeCN and purified by prep-LCMS (XBridge C18 column, eluting with a gradient of acetonitrile/water containing 0.10% TFA, at flow rate of 60 mL/min). LCMS calculated for C₃₁H₃₃D₄N₈O₆(M+H)⁺: m/z=621.3; found: 621.3.

### Step 5. Methyl ((1R,3R)-3-(6-((1-(1-carbamoylcyclobutyl)-4-(4-oxocyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

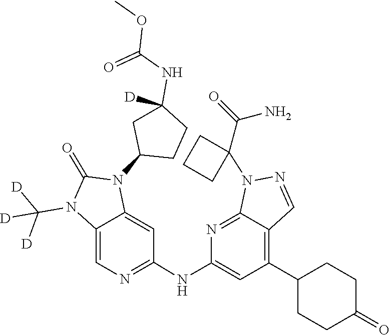 [SMILES: [2H]C([2H])([2H])n1c(=O)n([C@@H]2CC[C@@]([2H])(NC(=O)OC)C2)c2cc(Nc3cc(C4CCC(=O)CC4)c4cnn(C5(C(N)=O)CCC5)c4n3)ncc21]

To a vial containing 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(4-oxocyclohexyl)-1H-pyrazolo[3,4-b]pyridin-1-yl)cyclobutane-1-carboxylic acid (50 mg, 0.075 mmol) and ammonia (0.5 M in dioxane, 0.752 mL, 0.376 mmol) as a solution in DMF (0.3 mL) and DIPEA (0.066 mL, 0.376 mmol) was added HATU (43 mg, 0.113 mmol) and left to stir at r.t. for 30 min. The crude residue was dissolved in MeCN and purified by prep-LCMS (XBridge C18 column, eluting with a gradient of acetonitrile/water containing 0.1% TFA, at flow rate of 60 mL/min). LCMS calculated for C₃₁H₃₄D₄N₉O₅(M+H)⁺: m/z=620.3; found: 620.3.

### Step 6. Methyl ((1R,3R)-3-(6-((1-(1-carbamoylcyclobutyl)-4-((1r,4R)-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

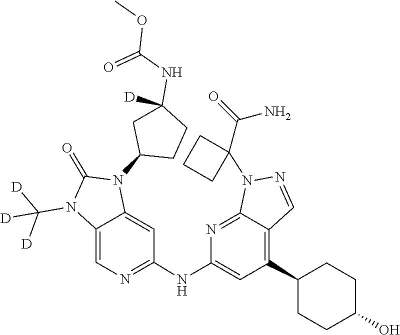 [SMILES: [2H]C([2H])([2H])n1c(=O)n([C@@H]2CC[C@@]([2H])(NC(=O)OC)C2)c2cc(Nc3cc([C@H]4CC[C@H](O)CC4)c4cnn(C5(C(N)=O)CCC5)c4n3)ncc21]

To a solution of methyl ((1R,3R)-3-(6-((1-(1-carbamoylcyclobutyl)-4-(4-oxocyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate (10 mg, 0.016 mmol) in MeOH (1.0 ml) was added sodium borohydride (0.7 mg, 0.019 mmol) at 0° C. and the reaction mixture was stirred at r.t. for 2 hrs. The crude residue was diluted with MeCN and purified by prep-LCMS (XBridge C18 column, eluting with a gradient of acetonitrile/water containing 0.1% TFA, at flow rate of 60 mL/min, the desired product was collected as major peak at Rt=11.1 min). LCMS calculated for C₃₁H₃₆D₄N₉O₅(M+H)⁺: m/z=622.3; found: 622.3. ¹H NMR (600 MHz, DMSO-d6) δ 8.34 (s, 1H), 8.24 (s, 1H), 7.43 (s, 1H), 7.37 (s, 1H), 6.79 (s, 1H), 5.03 (p, J=8.5 Hz, 1H), 3.60-3.52 (m, 1H), 3.55 (s, 3H), 2.97-2.81 (m, 5H), 2.36-2.30 (m, 1H), 2.24-2.11 (m, 2H), 2.09-1.91 (m, 8H), 1.69-1.59 (m, 3H), 1.46-1.37 (m, 2H) ppm.

### Example 14. Methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

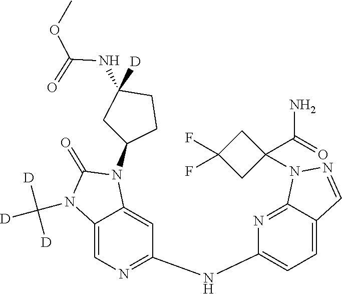 [SMILES: [2H]C([2H])([2H])n1c(=O)n([C@@H]2CC[C@@]([2H])(NC(=O)OC)C2)c2cc(Nc3ccc4cnn(C5(C(N)=O)CC(F)(F)C5)c4n3)ncc21]

### Step 1. Methyl 1-((6-bromo-3-cyanopyridin-2-yl)amino)-3,3-difluorocyclobutane-1-carboxylate

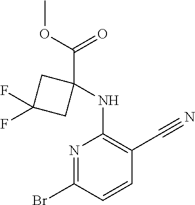 [SMILES: COC(=O)C1(Nc2nc(Br)ccc2C#N)CC(F)(F)C1]

To a reaction vial containing a solution of methyl 1-amino-3,3-difluorocyclobutane-1-carboxylate hydrochloride (176 mg, 0.87 mmol) and 6-bromo-2-fluoronicotinonitrile (175 mg, 0.87 mmol) in DMSO (0.3 mL) was added N,N-diisopropylethylamine (350 μL, 2.0 mmol). The reaction mixture was heated to 100° C. and allowed to stir for 10 h, after which it was cooled to r.t. and diluted with EtOAc. The organic layer was subsequently washed with water and brine, dried over sodium sulfate, filtered, and the solvents were evaporated in vacuo. The crude material was purified by Biotage Isolera to give the product. LCMS calculated for C₁₂H₁₁BrF₂N₃O₂ (M+H)⁺: m/z=346.0; found 346.0.

### Step 2. Methyl 1-(6-bromo-1H-pyrazolo[3,4-b]pyridin-1-yl)-3,3-difluorocyclobutane-1-carboxylate

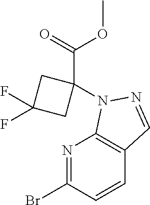 [SMILES: COC(=O)C1(n2ncc3ccc(Br)nc32)CC(F)(F)C1]

To a reaction vial containing a solution of methyl 1-((6-bromo-3-cyanopyridin-2-yl)amino)-3,3-difluorocyclobutane-1-carboxylate (205 mg, 0.6 mmol) in THF (0.8 mL) and DMF (1.6 mL) cooled to 0° C. was slowly added a lithium bis(trimethylsilyl)amide (770 μL, 0.77 mmol, 1M in THF) and allowed to stir for 10 min. Then (aminooxy)diphenylphosphine oxide (138 mg, 0.6 mmol) was added and the reaction was allowed to warm to r.t. After 1 h at r.t, the reaction was quenched with a saturated aqueous solution of ammonium chloride and extracted with diethyl ether. The organic layers were combined, dried over sodium sulfate, filtered, and the solvents were evaporated in vacuo. The crude material was added to a reaction vial containing THF (0.8 mL) and tert-butyl nitrite (282 μL, 2.37 mmol). The reaction mixture was stirred at 65° C. for 2 h, after which the solvent was removed in vacuo, and the crude material was purified by Biotage Isolera to give the product. LCMS calculated for Cl₂H₁₁BrF₂N₃O₂ (M+H)⁺: m/z=346.0; found 346.0.

### Step 3. 3,3-Difluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid

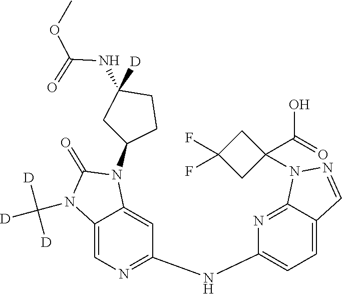 [SMILES: [2H]C([2H])([2H])n1c(=O)n([C@@H]2CC[C@@]([2H])(NC(=O)OC)C2)c2cc(Nc3ccc4cnn(C5(C(=O)O)CC(F)(F)C5)c4n3)ncc21]

To a reaction vial was added methyl ((1R,3R)-3-(6-amino-3-(methyl-d₃)-2-oxo-2,3-dihydro-1H-imidazo[4,5-c]pyridin-1-yl)cyclopentyl-1-d)carbamate (58 mg, 0.19 mmol, Intermediate A), methyl 1-(6-bromo-1H-pyrazolo[3,4-b]pyridin-1-yl)-3,3-difluorocyclobutane-1-carboxylate (65 mg, 0.19 mmol), XantPhos Pd G3 (18 mg, 19 mol), and cesium carbonate (183 mg, 0.56 mmol). This was evacuated and backfilled with nitrogen 3 times. Subsequently 1,4-dioxane (2 mL) was added. The vial was sealed and heated to 100° C. for 2 h. Upon completion, the reaction was diluted with DCM, filtered through Celite and concentrated in vacuo. The crude residue was dissolved in a reaction vial containing THF (1 mL), MeOH (1 mL), and lithium hydroxide (1 mL, 1M in H₂O), heated to 55° C., and allowed to stir for 4 h. Upon completion, the reaction was quenched with HCl (3 mL, 1M in H₂O) and extracted with 3:1 chloroform:isopropanol. The organic layers were combined, dried over sodium sulfate, filtered, and the solvents were evaporated in vacuo. The crude material was used directly in the next reaction. LCMS calculated for C₂₅H₂₃D₄F₂N₈₀₅ (M+H)⁺: m/z=561.2; found 561.2.

### Step 4. Methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a reaction vial containing 3,3-difluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid (105 mg, 0.19 mmol), HATU (142 mg, 0.38 mmol) and DIPEA (100 μL, 0.56 mmol) was added ammonia (1.3 mL, 7M in methanol). The reaction mixture was sealed and stirred at 60° C. for 2 h. Upon completion, the reaction was cooled to r.t. and solvent was removed in vacuo. The reaction residue was diluted with MeCN and purified by prep-LCMS (XBridge C18 column, eluting with a gradient 21→41% of acetonitrile/water containing 0.1% TFA, at flow rate of 60 mL/min.) Retention time=4.9 min LCMS calculated for C₂₅H₂₄D₄F₂N₉O₄ (M+H)⁺: m/z=560.3; found 560.3. ¹H NMR (600 MHz, DMSO) δ 11.35 (s, 1H), 8.28 (s, 1H), 8.25 (d, J=8.5 Hz, 1H), 8.21 (s, 1H), 7.68 (s, 1H), 7.59-7.38 (m, 2H), 7.32 (s, 1H), 7.04 (d, J=8.7 Hz, 1H), 5.05-4.96 (m, 1H), 3.66 (q, J=12.5 Hz, 4H), 3.54 (s, 3H), 2.34 (dd, J=13.7, 7.6 Hz, 1H), 2.24-2.11 (m, 2H), 2.10-2.01 (m, 1H), 1.95 (dd, J=13.7, 9.6 Hz, 1H), 1.68-1.60 (m, 1H).

### Example 15. Methyl ((1R,3R)-3-(6-((1-(1-carbamoyl-3-methoxycyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

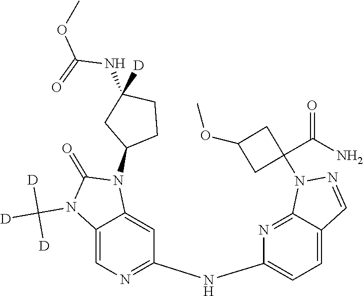 [SMILES: [2H]C([2H])([2H])n1c(=O)n([C@@H]2CC[C@@]([2H])(NC(=O)OC)C2)c2cc(Nc3ccc4cnn(C5(C(N)=O)CC(OC)C5)c4n3)ncc21]

This compound was prepared according to the procedures described in Example 14, Steps 1-5 with methyl 1-amino-3-methoxycyclobutane-1-carboxylate hydrochloride replacing methyl 1-amino-3,3-difluorocyclobutane-1-carboxylate hydrochloride in Step 1, and final purification by prep-LCMS (XBridge C18 column, eluting with a gradient of 30→50% acetonitrile/aqueous 0.1% NH₄OH, at flow rate of 60 mL/min.) The product was obtained as a single diastereomer. The desired diastereomer was collected at a retention time of 3.3 min. LCMS calculated for C₂₆H₂₈D₄N₉O₅(M+H)⁺: m/z=554.3; found 554.3.

### Example 16. Methyl ((1R,3R)-3-(6-((1-(1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

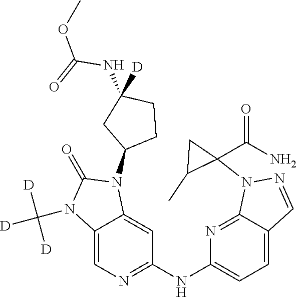 [SMILES: [2H]C([2H])([2H])n1c(=O)n([C@@H]2CC[C@@]([2H])(NC(=O)OC)C2)c2cc(Nc3ccc4cnn(C5(C(N)=O)CC5C)c4n3)ncc21]

This compound was prepared according to the procedures described in Example 14, Steps 1-5 with rac-methyl (1R,2R)-1-amino-2-methylcyclopropane-1-carboxylate hydrochloride replacing methyl 1-amino-3,3-difluorocyclobutane-1-carboxylate hydrochloride in Step 1, and final purification by prep-LCMS (XBridge C18 column, eluting with a gradient of 28→48% acetonitrile/aqueous 0.1% NH₄OH, at flow rate of 60 mL/min.) The desired diastereomer was collected at a retention time of 4.2 min. LCMS calculated for C₂₅H₂₆D₄N₉O₄(M+H)⁺: m/z=524.3; found 524.3. ¹H NMR (500 MHz, DMSO) δ 9.99 (s, 1H), 8.46 (s, 1H), 8.06 (s, 1H), 7.94 (d, J=8.7 Hz, 1H), 7.93 (s, 1H), 7.33 (s, 1H), 7.24 (s, 1H), 7.09 (d, J=8.7 Hz, 1H), 6.41 (s, 1H), 4.92-4.84 (m, 1H), 3.51 (s, 3H), 2.44 (dd, J=13.3, 7.5 Hz, 1H), 2.26-2.17 (m, 2H), 2.16-2.02 (m, 1H), 1.93 (dd, J=13.3, 9.3 Hz, 1H), 1.74 (dd, J=8.2, 4.8 Hz, 1H), 1.65 (m, 2H), 1.46 (dd, J=9.6, 4.7 Hz, 1H), 1.33 (d, J=6.3 Hz, 3H).

### Example 17. Methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

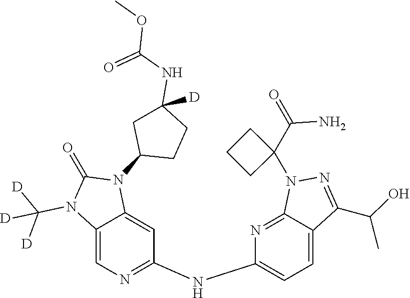 [SMILES: [2H]C([2H])([2H])n1c(=O)n([C@@H]2CC[C@@]([2H])(NC(=O)OC)C2)c2cc(Nc3ccc4c(C(C)O)nn(C5(C(N)=O)CCC5)c4n3)ncc21]

### Step 1. Ethyl 1-(6-bromo-1H-pyrazolo[3,4-b]pyridin-1-yl)cyclobutane-1-carboxylate

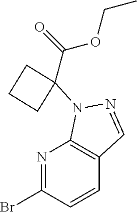 [SMILES: CCOC(=O)C1(n2ncc3ccc(Br)nc32)CCC1]

To a reaction flask containing a solution of ethyl 1-hydrazineylcyclobutane-1-carboxylate hydrochloride (3.8 g, 19.6 mmol) and 6-bromo-2-fluoronicotinaldehyde (4.0 g, 19.6 mmol) in N-methyl-2-pyrrolidone (30 mL) was added N,N-diisopropylethylamine (8.6 mL, 49.0 mmol). The reaction mixture was heated to 180° C. and allowed to stir for 3 h, after which it was cooled to r.t. and diluted with EtOAc. The organic layer was subsequently washed with an aqueous solution of lithium chloride and brine, dried over sodium sulfate, filtered, and the solvents were evaporated in vacuo. The crude material was purified by Biotage Isolera to give the product. LCMS calculated for C₁₃H₁₅BrN₃O₂(M+H)⁺: m/z=324.0; found 324.0.

### Step 2. Ethyl 1-(6-bromo-3-iodo-1H-pyrazolo[3,4-b]pyridin-1-yl)cyclobutane-1-carboxylate

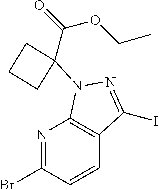 [SMILES: CCOC(=O)C1(n2nc(I)c3ccc(Br)nc32)CCC1]

To a flask containing ethyl 1-(6-bromo-1H-pyrazolo[3,4-b]pyridin-1-yl)cyclobutane-1-carboxylate (4.6 g, 14.3 mmol) in acetonitrile (100 mL) was added N-iodosuccinimide (3.5 g, 15.7 mmol) and tetrafluoroboric acid (9.3 mL, 71.3 mmol, 48% in H₂O). The reaction mixture was heated to 80° C. and stirred for 1 h, upon which it was cooled to r.t. and quenched with saturated aqueous solutions of NaHCO₃ and Na₂S₂O₃. The mixture was extracted with EtOAc and the organic layer was subsequently dried over sodium sulfate, filtered, and the solvents were evaporated in vacuo. The crude material was purified by Biotage Isolera to give the product. LCMS calculated for C₁₃H₁₄BrIN₃O₂ (M+H)⁺: m/z=449.9; found 449.9.

### Step 3. Ethyl 1-(6-bromo-3-vinyl-1H-pyrazolo[3,4-b]pyridin-1-yl)cyclobutane-1-carboxylate

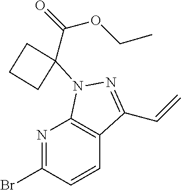 [SMILES: C=Cc1nn(C2(C(=O)OCC)CCC2)c2nc(Br)ccc12]

The reaction vial containing ethyl 1-(6-bromo-3-iodo-1H-pyrazolo[3,4-b]pyridin-1-yl)cyclobutane-1-carboxylate (1.0 g, 2.22 mmol), 4,4,5,5-tetramethyl-2-vinyl-1,3,2-dioxaborolane (0.4 mL, 2.33 mmol), cesium carbonate (1.1 g, 3.33 mmol) and [1,1′-bis(diphenylphosphino)ferrocene]-dichloropalladium(II) (363 mg, 0.44 mmol) was evacuated and backfilled with nitrogen. 1,4-Dioxane (6 mL) and water (1 mL) were added to the reaction mixture, which was then stirred at 55° C. for 8 h. The reaction mixture was cooled to r.t. and was diluted with EtOAc. The organic layer was subsequently washed with water and brine, dried over sodium sulfate, filtered, and the solvents were evaporated in vacuo. The crude material was purified by Biotage Isolera. LCMS calculated for: C₁₅H₁₇BrN₃O₂(M+H)⁺: m/z=350.1; found 350.1.

### Step 4. Ethyl 1-(6-bromo-3-formyl-1H-pyrazolo[3,4-b]pyridin-1-yl)cyclobutane-1-carboxylate

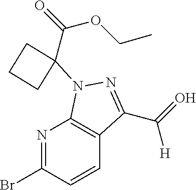 [SMILES: CCOC(=O)C1(n2nc(C=O)c3ccc(Br)nc32)CCC1]

A reaction vial containing ethyl 1-(6-bromo-3-vinyl-1H-pyrazolo[3,4-b]pyridin-1-yl)cyclobutane-1-carboxylate (546 mg, 1.56 mmol) in dichloromethane (6 mL) was cooled to −78° C. Ozone was bubbled through the solution until the solution turned light blue in color after 5 min. Dimethyl sulfide (0.58 mL, 7.8 mmol) was added to the solution, which was then allowed to warm to r.t. The solvents were removed in vacuo and the crude product was used directly in the next step without further purification. LCMS calculated for: C₁₄H₁₅BrN₃O₃ (M+H)⁺: m/z=352.0; found 352.0.

### Step 5. Ethyl 1-(6-bromo-3-(1-hydroxyethyl)-1H-pyrazolo[3,4-b]pyridin-1-yl)cyclobutane-1-carboxylate

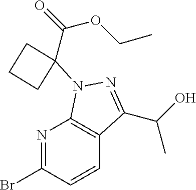 [SMILES: CCOC(=O)C1(n2nc(C(C)O)c3ccc(Br)nc32)CCC1]

To a reaction vial containing ethyl 1-(6-bromo-3-formyl-1H-pyrazolo[3,4-b]pyridin-1-yl)cyclobutane-1-carboxylate (549 mg, 1.56 mmol) in THF (4 mL) was added dropwise methylmagnesium bromide (0.52 mL, 1.56 mmol, 3.0 M in Et₂O) at −78° C. The reaction was allowed to warm to r.t. and stirred for 30 min. The mixture was quenched with sat. NH₄Cl and extracted with EtOAc. The organic layers were combined, dried over Na₂SO₄, filtered, and the solvents were evaporated in vacuo. The crude material was purified by Biotage Isolera. LCMS calculated for: C₁₅H₁₉BrN₃O₃(M+H)⁺: m/z=368.1; found 368.1.

### Step 6. Methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

This compound was prepared according to the procedure in Example 14, Steps 3-4, with ethyl 1-(6-bromo-3-(1-hydroxyethyl)-1H-pyrazolo[3,4-b]pyridin-1-yl)cyclobutane-1-carboxylate replacing methyl 1-(6-bromo-1H-pyrazolo[3,4-b]pyridin-1-yl)-3,3-difluorocyclobutane-1-carboxylate in Step 3. The final product was obtained as a mixture of two diastereomers. LCMS calculated for: C₂₇H₃₀D₄N₉O₅ (M+H)⁺: m/z=568.3; found 568.3.

### Example 18. Methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

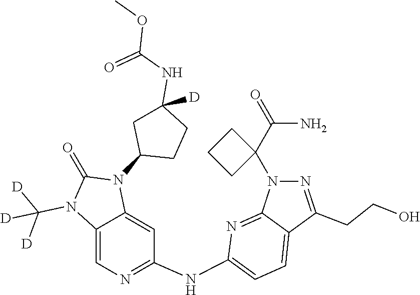 [SMILES: [2H]C([2H])([2H])n1c(=O)n([C@@H]2CC[C@@]([2H])(NC(=O)OC)C2)c2cc(Nc3ccc4c(CCO)nn(C5(C(N)=O)CCC5)c4n3)ncc21]

### Step 1. Ethyl (E)-1-(6-bromo-3-(2-ethoxyvinyl)-1H-pyrazolo[3,4-b]pyridin-1-yl)cyclobutane-1-carboxylate

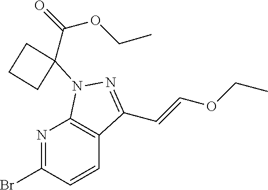 [SMILES: CCO/C=C/c1nn(C2(C(=O)OCC)CCC2)c2nc(Br)ccc12]

This compound was prepared according to the procedure in Example 17, Step 3, with (E)-2-(2-ethoxyvinyl)-4,4,5,5-tetramethyl-1,3,2-dioxaborolane replacing 4,4,5,5-tetramethyl-2-vinyl-1,3,2-dioxaborolane. LCMS calculated for: C₁₇H₂₁BrN₃O₃(M+H)⁺: m/z=394.1; found 394.1.

### Step 2. Ethyl 1-(3-((E)-2-ethoxyvinyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

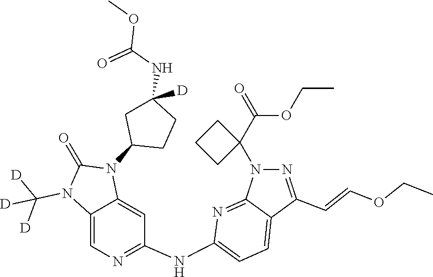 [SMILES: [2H]C([2H])([2H])n1c(=O)n([C@@H]2CC[C@@]([2H])(NC(=O)OC)C2)c2cc(Nc3ccc4c(/C=C/OCC)nn(C5(C(=O)OCC)CCC5)c4n3)ncc21]

To a reaction vial was added methyl ((1R,3R)-3-(6-amino-3-(methyl-d₃)-2-oxo-2,3-dihydro-1H-imidazo[4,5-c]pyridin-1-yl)cyclopentyl-1-d)carbamate (190 mg, 0.61 mmol, Intermediate A), ethyl (E)-1-(6-bromo-3-(2-ethoxyvinyl)-1H-pyrazolo[3,4-b]pyridin-1-yl)cyclobutane-1-carboxylate (242 mg, 0.61 mmol), tris(dibenzylideneacetone)dipalladium (84 mg, 92 μmol), 9,9-dimethyl-9H-xanthene-4,5-diyl)bis(diphenylphosphane (107 mg, 0.18 mmol), and cesium carbonate (600 mg, 1.84 mmol). This was evacuated and backfilled with nitrogen 3 times. Subsequently 1,4-dioxane (3 mL) was added. The vial was sealed and heated to 100° C. for 2 h. Upon completion, the reaction was cooled to r.t., diluted with DCM, filtered through Celite and concentrated in vacuo. The organic layers were combined, dried over sodium sulfate, filtered, and the solvents were evaporated in vacuo. The crude material was purified by Biotage Isolera to give the product. LCMS calculated for: C₃₁H₃₅D₄N₈O₆ (M+H)⁺: m/z=623.3; found 623.3.

### Step 3. Ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(2-oxoethyl)-1H-pyrazolo[3,4-b]pyridin-1-yl)cyclobutane-1-carboxylate

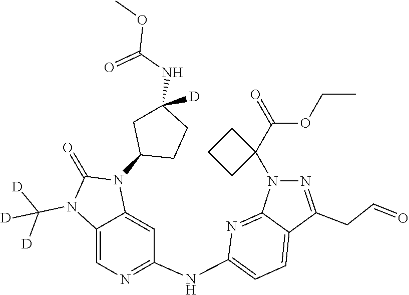 [SMILES: [2H]C([2H])([2H])n1c(=O)n([C@@H]2CC[C@@]([2H])(NC(=O)OC)C2)c2cc(Nc3ccc4c(CC=O)nn(C5(C(=O)OCC)CCC5)c4n3)ncc21]

To a reaction vial containing ethyl 1-(3-((E)-2-ethoxyvinyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (322 mg, 0.52 mmol) in dichloromethane (2 mL) was added trifluoroacetic acid (2 mL). The reaction was heated to 35° C. and allowed to stir for 2 h, upon which the solvents were removed in vacuo. The crude residue was extracted with dichloromethane and washed with saturated aqueous NaHCO₃. The organic layers were combined, dried over Na₂SO₄, filtered, and solvents were removed in vacuo. The crude product was used directly in the next step without further purification. LCMS calculated for: C₂₉H₃₁D₄N₈O₆(M+H)⁺: m/z=595.3; found 595.3.

### Step 4. Ethyl 1-(3-(2-hydroxyethyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

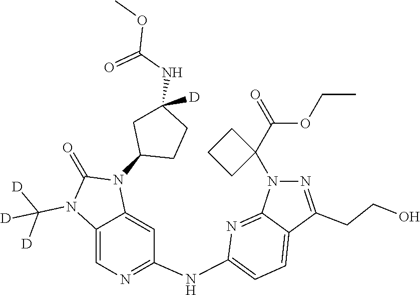 [SMILES: [2H]C([2H])([2H])n1c(=O)n([C@@H]2CC[C@@]([2H])(NC(=O)OC)C2)c2cc(Nc3ccc4c(CCO)nn(C5(C(=O)OCC)CCC5)c4n3)ncc21]

To a via containing ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(2-oxoethyl)-1H-pyrazolo[3,4-b]pyridin-1-yl)cyclobutane-1-carboxylate (25 mg, 42 μmol) in methanol (0.5 mL) was added sodium borohydride (8 mg, 0.2 mmol). After stirring at r.t. for 30 min, the reaction mixture was quenched with water, and extracted with EtOAc. The organic layers were combined, dried over Na₂SO₄, filtered, and solvents were removed in vacuo. The crude material was purified by Biotage Isolera to give the product. LCMS calculated for: C₂₉H₃₃D₄N₈O₆(M+H)⁺: m/z=597.3; found 597.3.

### Step 5. Methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a reaction vial containing ethyl 1-(3-(2-hydroxyethyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (25 mg, 42 mol) in THF (1 mL) and MeOH (1 mL) was added lithium hydroxide (1 mL, 1 M in H₂O). The reaction mixture was heated to 50° C. and allowed to stir for 1 h, after which the reaction was quenched with HCl (3 mL, 1 M in H₂O) and extracted with 3:1 chloroform:isopropanol. The organic layers were combined, dried over sodium sulfate, filtered, and the solvents were evaporated in vacuo. The crude material was added to a reaction vial containing HATU (24 mg, 63 mol) and DIPEA (22 μL, 0.13 mmol) in DMF (1 mL). Ammonia (1.3 mL, 7 M in methanol) was added to this reaction mixture, and it was then sealed and stirred at r.t. for 1 h. Upon completion, a nitrogen gas inlet needle was inserted to blow off excess ammonia. The reaction residue was diluted with MeCN and purified by prep-LCMS (XBridge C18 column, eluting with a gradient 21→41% of acetonitrile/water containing 0.1% TFA, at flow rate of 60 mL/min.) Retention time=3.9 min. LCMS calculated for: C₂₇H₃₀D₄N₉O₅ (M+H)⁺: m/z=568.3; found 568.3. ¹H NMR (600 MHz, DMSO) δ 11.51 (s, 1H), 8.29 (s, 1H), 8.28 (s, 1H), 7.46 (s, 1H), 7.43-7.26 (m, 3H), 6.93 (d, J=8.6 Hz, 1H), 5.07-4.98 (m, 1H), 3.82 (t, J=6.7 Hz, 2H), 3.55 (s, 3H), 3.08 (t, J=6.7 Hz, 2H), 2.95-2.81 (m, 4H), 2.33 (dd, J=13.7, 7.8 Hz, 1H), 2.25-2.11 (m, 2H), 2.10-2.00 (m, 1H), 2.00-1.90 (m, 3H), 1.69-1.62 (m, 1H).

### Example 19. Methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

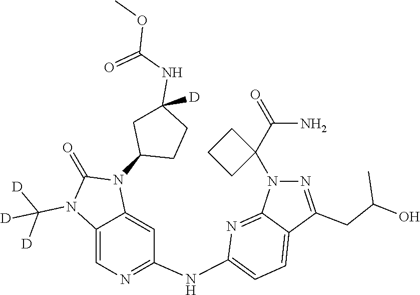 [SMILES: [2H]C([2H])([2H])n1c(=O)n([C@@H]2CC[C@@]([2H])(NC(=O)OC)C2)c2cc(Nc3ccc4c(CC(C)O)nn(C5(C(N)=O)CCC5)c4n3)ncc21]

### Step 1. Ethyl 1-(3-(2-hydroxypropyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

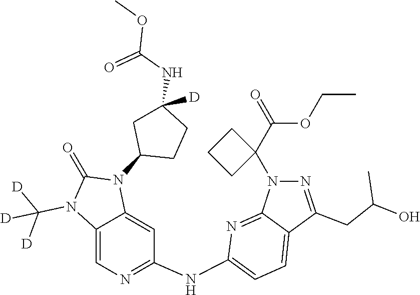 [SMILES: [2H]C([2H])([2H])n1c(=O)n([C@@H]2CC[C@@]([2H])(NC(=O)OC)C2)c2cc(Nc3ccc4c(CC(C)O)nn(C5(C(=O)OCC)CCC5)c4n3)ncc21]

This compound was prepared according to the procedure in Example 17, Step 5, with ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(2-oxoethyl)-1H-pyrazolo[3,4-b]pyridin-1-yl)cyclobutane-1-carboxylate replacing ethyl 1-(6-bromo-3-formyl-1H-pyrazolo[3,4-b]pyridin-1-yl)cyclobutane-1-carboxylate. LCMS calculated for: C₃₀H₃₅D₄N₈O₆ (M+H)⁺: m/z=611.3; found 611.3.

### Step 2. Methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

This compound was prepared according to the procedure in Example 18, Step 5, with ethyl 1-(3-(2-hydroxypropyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate replacing ethyl 1-(3-(2-hydroxyethyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate. The final product was obtained as a mixture of diastereomers. LCMS calculated for: C₂₈H₃₂D₄N₉O₅(M+H)⁺: m/z=582.3; found 582.3.

### Example 20. Methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

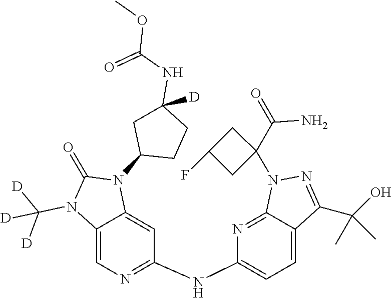 [SMILES: [2H]C([2H])([2H])n1c(=O)n([C@@H]2CC[C@@]([2H])(NC(=O)OC)C2)c2cc(Nc3ccc4c(C(C)(C)O)nn(C5(C(N)=O)CC(F)C5)c4n3)ncc21]

### Step 1. Isopropyl 1-(6-bromo-3-(prop-1-en-2-yl)-1H-pyrazolo[3,4-b]pyridin-1-yl)-3-fluorocyclobutane-1-carboxylate

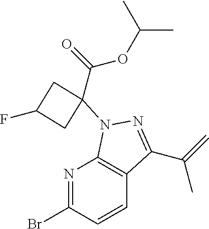 [SMILES: C=C(C)c1nn(C2(C(=O)OC(C)C)CC(F)C2)c2nc(Br)ccc12]

This compound was prepared according to the procedure in Example 17, Steps 2-3, with isopropyl 1-(6-bromo-1H-pyrazolo[3,4-b]pyridin-1-yl)-3-fluorocyclobutane-1-carboxylate (Example 24, Step 6) replacing ethyl 1-(6-bromo-1H-pyrazolo[3,4-b]pyridin-1-yl)cyclobutane-1-carboxylate in Step 2, and with 4,4,5,5-tetramethyl-2-(prop-1-en-2-yl)-1,3,2-dioxaborolane replacing 4,4,5,5-tetramethyl-2-vinyl-1,3,2-dioxaborolane in Step 3. LCMS calculated for: C₁₇H₂₀BrFN₃O₂(M+H)⁺: m/z=396.1; found 396.1.

### Step 2. Isopropyl 1-(6-bromo-3-(2-hydroxypropan-2-yl)-1H-pyrazolo[3,4-b]pyridin-1-yl)-3-fluorocyclobutane-1-carboxylate

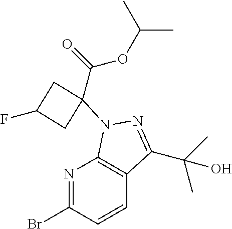 [SMILES: CC(C)OC(=O)C1(n2nc(C(C)(C)O)c3ccc(Br)nc32)CC(F)C1]

To a reaction vial containing isopropyl 1-(6-bromo-3-(prop-1-en-2-yl)-1H-pyrazolo[3,4-b]pyridin-1-yl)-3-fluorocyclobutane-1-carboxylate (55 mg, 0.14 mmol), Fe(acac)₃ (5 mg, 14 mmol), methyl 4-nitrobenzenesulfonate (45 mg, 0.21 mmol), and NaHCO₃ (35 mg, 0.42 mmol) was added methanol (1 mL). The reaction mixture was cooled to 0° C. and allowed to stir for 5 min, after which phenylsilane (21 μL, 0.17 mmol) was added dropwise and the reaction was allowed to stir open to air at r.t. for 1 h. The solvents were removed in vacuo and the crude material was purified by Biotage Isolera. LCMS calculated for: C₁₇H₂₂BrFN₃O₃(M+H)⁺: m/z=414.1; found 414.1.

### Step 3. Methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

This compound was prepared according to the procedure in Example 14, Steps 3-4, with isopropyl 1-(6-bromo-3-(2-hydroxypropan-2-yl)-1H-pyrazolo[3,4-b]pyridin-1-yl)-3-fluorocyclobutane-1-carboxylate replacing methyl 1-(6-bromo-1H-pyrazolo[3,4-b]pyridin-1-yl)-3,3-difluorocyclobutane-1-carboxylate in Step 3, and final purification by prep-LCMS (XBridge C18 column, eluting with a gradient of 35→55% acetonitrile/aqueous 0.1% NH₄OH, at flow rate of 60 mL/min.). The product was isolated as a single isomer. Retention time=3.2. LCMS calculated for: C₂₈H₃₁D₄FN₉O₅(M+H)⁺: m/z=600.3; found 600.3.

### Example 21. Methyl ((1R,3R)-3-(6-((3-(1-carbamoyl-3-fluorocyclobutyl)-7-methyl-2-(trifluoromethyl)-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

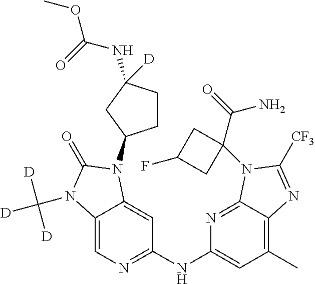 [SMILES: [2H]C([2H])([2H])n1c(=O)n([C@@H]2CC[C@@]([2H])(NC(=O)OC)C2)c2cc(Nc3cc(C)c4nc(C(F)(F)F)n(C5(C(N)=O)CC(F)C5)c4n3)ncc21]

This compound was prepared according to the procedures described in Example 22, Step 1 to 8, with trifluoroacetic anhydride replacing 2,2-difluoroacetic anhydride in Step 3. The crude product was purified by prep-LCMS as the second peak of two diastereoisomers (XBridge C18 column, eluting with a gradient of 16.9→34.8% acetonitrile in water containing 0.1% TFA, at flow rate of 60 mL/min over 12 min). The desired product was collected at a retention time of 10.9 min as a single diastereomer. LCMS calculated for C₂₇H₂₆D₄F₄N₉O₄ (M+H)⁺: m/z=624.3; found 624.3.

### Example 22. Methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

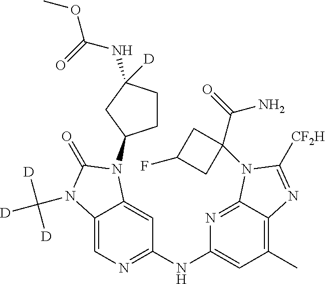 [SMILES: [2H]C([2H])([2H])n1c(=O)n([C@@H]2CC[C@@]([2H])(NC(=O)OC)C2)c2cc(Nc3cc(C)c4nc(C(F)F)n(C5(C(N)=O)CC(F)C5)c4n3)ncc21]

### Step 1. (1-((6-Chloro-4-methyl-3-nitropyridin-2-yl)amino)-3-fluorocyclobutyl)methanol

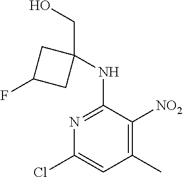 [SMILES: Cc1cc(Cl)nc(NC2(CO)CC(F)C2)c1[N+](=O)[O-]]

To a vial containing 2,6-dichloro-4-methyl-3-nitropyridine (270 mg, 1.28 mmol) and (1-amino-3-fluorocyclobutyl)methanol hydrochloride (200 mg, 1.28 mmol) in DMSO (0.43 mL) was added DIPEA (450 μL, 2.6 mmol). After heating at 65° C. for 12 h, the reaction mixture was directly purified by Biotage Isolera to give the desired product as yellow solid. LCMS calculated for C₁₁H₁₄ClFN₃O₃(M+H)⁺: m/z=290.1; found 290.1.

### Step 2. (1-((3-Amino-6-chloro-4-methylpyridin-2-yl)amino)-3-fluorocyclobutyl)methanol

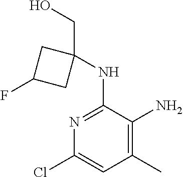 [SMILES: Cc1cc(Cl)nc(NC2(CO)CC(F)C2)c1N]

A vial containing a suspension of zinc dust (590 mg, 9.0 mmol) and NH₄Cl (480 mg, 9.0 mmol) in MeOH (10 mL) was placed in a water bath. A solution of (1-((6-chloro-4-methyl-3-nitropyridin-2-yl)amino)-3-fluorocyclobutyl)methanol (260 mg, 0.90 mmol) in CH₂Cl₂ (2 mL) was added dropwise to the zinc powder suspension with vigorous stirring at the rate of 2 drop/sec. After stirring at r.t for 20 min, the reaction mixture was filtered and washed with CH₂Cl₂ (8 mL) three times. The filtrate was collected and concentrated. The residue was dissolved in CH₂Cl₂ (20 mL), washed with water (5 mL), and extracted with CH₂Cl₂. The organic phase was washed with brine, dried over MgSO₄ and concentrated in vacuo. The obtained crude product was used in the next step without further purification. LCMS calculated for C₁₁H₁₆ClFN₃O (M+H)⁺: m/z=260.1; found 260.1.

### Step 3. N-(6-Chloro-2-((3-fluoro-1-(hydroxymethyl)cyclobutyl)amino)-4-methylpyridin-3-yl)-2,2-difluoroacetamide

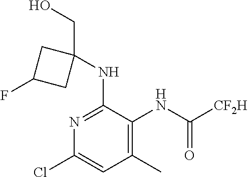 [SMILES: Cc1cc(Cl)nc(NC2(CO)CC(F)C2)c1NC(=O)C(F)F]

To a vial containing (1-((3-amino-6-chloro-4-methylpyridin-2-yl)amino)-3-fluorocyclobutyl)methanol (100 mg, 0.38 mmol) in CH₂Cl₂ (2.0 mL) was added DIPEA (0.34 mL, 1.93 mmol) and 2,2-difluoroacetic anhydride (200 mg, 1.16 mmol). After stirring at r.t for 20 min, the reaction mixture was quenched with MeOH (1.0 mL) and concentrated in vacuo. The crude mixture was dissolved in MeOH/THF/water (1.5 mL, v/v/v=1/1/1), followed by the addition of LiOH (18 mg, 0.78 mmol). After heating at 50° C. for 15 min, the reaction mixture was cooled to r.t. and extracted with CH₂Cl₂ (3×5 mL). The organic layers were combined, dried over MgSO₄ and concentrated in vacuo. The obtained crude product was used in the next step without further purification. LCMS calculated for C₁₃H₁₆CF₃N₃O₂ (M+H)⁺: m/z=338.1; found 338.1.

### Step 4. (1-(5-Chloro-2-(difluoromethyl)-7-methyl-3H-imidazo[4,5-b]pyridin-3-yl)-3-fluorocyclobutyl)methanol

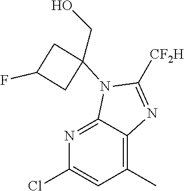 [SMILES: Cc1cc(Cl)nc2c1nc(C(F)F)n2C1(CO)CC(F)C1]

To a vial containing crude N-(6-chloro-2-((3-fluoro-1-(hydroxymethyl)cyclobutyl)-amino)-4-methylpyridin-3-yl)-2,2-difluoroacetamide (130 mg, 0.38 mmol) was added acetic acid (1.0 ml). After stirring at 100° C. for 14 h, the reaction mixture was diluted with EtOAc (3 mL) quenched with sat. aq. NaHCO₃ solution, followed by extraction with EtOAc (3×10 mL). The organic layers were combined, dried over MgSO₄ and concentrated in vacuo. The crude mixture was dissolved in MeOH/THF/water (1.5 mL, v/v/v=1/1/1), followed by the addition of LiOH (18 mg, 0.78 mmol). After heating at 50° C. for 15 min, the reaction mixture was cooled to r.t. and extracted with CH₂Cl₂ (3×5 mL). The organic layer was combined, dried over MgSO₄ and concentrated in vacuo. The obtained crude product was used in the next step without further purification. LCMS calculated for C₁₃H₁₄ClF₃N₃O (M+H)⁺: m/z=320.1; found 320.2.

### Step 5. 1-(5-Chloro-2-(difluoromethyl)-7-methyl-3H-imidazo[4,5-b]pyridin-3-yl)-3-fluorocyclobutane-1-carboxylic acid

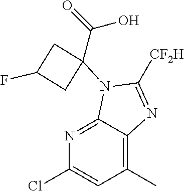 [SMILES: Cc1cc(Cl)nc2c1nc(C(F)F)n2C1(C(=O)O)CC(F)C1]

To a vial containing (1-(5-chloro-2-(difluoromethyl)-7-methyl-3H-imidazo[4,5-b]pyridin-3-yl)-3-fluorocyclobutyl)methanol (121 mg, 0.38 mmol) in CH₂Cl₂/EtOAc/water (3.0 mL, v/v/v=1/1/1) was added sodium periodate (250 mg, 1.16 mmol) and ruthenium(III) chloride (1 mg, 4 μmol). The reaction mixture was stirred vigorously and heated at 45° C. for 40 min. The reaction mixture was cooled down to r.t. and extracted with EtOAc (3×4 mL). The organic layers were combined, dried over MgSO₄ and concentrated in vacuo. The obtained crude product was used in the next step without further purification. LCMS calculated for C₁₃H₁₂ClF₃N₃O₂ (M+H)⁺: m/z=334.0; found 334.1.

### Step 6. Methyl 1-(5-chloro-2-(difluoromethyl)-7-methyl-3H-imidazo[4,5-b]pyridin-3-yl)-3-fluorocyclobutane-1-carboxylate

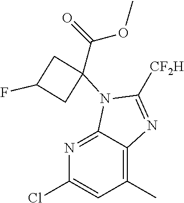 [SMILES: COC(=O)C1(n2c(C(F)F)nc3c(C)cc(Cl)nc32)CC(F)C1]

To a vial containing 1-(5-chloro-2-(difluoromethyl)-7-methyl-3H-imidazo[4,5-b]pyridin-3-yl)-3-fluorocyclobutane-1-carboxylic acid (127 mg, 0.38 mmol) and potassium carbonate (106 mg, 0.77 mmol) in acetonitrile (2.0 mL) was added iodomethane (72 μL, 1.16 mmol). The reaction mixture was heated at 50° C. for 1 h. The reaction mixture was cooled down to r.t., quenched with sat. aq. NH₄Cl solution (3 mL) and extracted with EtOAc (3×4 mL). The organic layers were combined, dried over MgSO₄ and concentrated in vacuo. The crude material was purified by Biotage Isolera to give the desired product as white solid. LCMS calculated for C₁₄H₁₄ClF₃N₃O₂ (M+H)⁺: m/z=348.1; found 348.1.

### Step 7. 1-(2-(Difluoromethyl)-5-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-7-methyl-3H-imidazo[4,5-b]pyridin-3-yl)-3-fluorocyclobutane-1-carboxylic acid

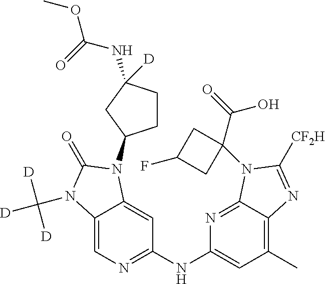 [SMILES: [2H]C([2H])([2H])n1c(=O)n([C@@H]2CC[C@@]([2H])(NC(=O)OC)C2)c2cc(Nc3cc(C)c4nc(C(F)F)n(C5(C(=O)O)CC(F)C5)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (21 mg, 67 μmol, Intermediate A), methyl 1-(5-chloro-2-(difluoromethyl)-7-methyl-3H-imidazo[4,5-o]pyridin-3-yl)-3-fluorocyclobutane-1-carboxylate (23 mg, 67 μmol), tris(dibenzylideneacetone)-dipalladium(0) (9 mg, 9.6 μmol), 4,5-bis(diphenylphosphino)-9,9-dimethylxanthene (11 mg, 19 μmol) and cesium carbonate (44 mg, 134 μmol) was evacuated and backfilled with nitrogen three times, followed by the addition of 1,4-dioxane (0.6 mL). The vial was sealed and heated to 100° C. for 16 h. After cooling to r.t., the mixture was diluted with MeOH (0.6 mL) and water (0.6 mL), followed by the addition of LiOH (16 mg, 0.67 mmol). After heating at 50° C. for 15 min, the reaction mixture was cooled to r.t. and diluted with CH₂Cl₂ (3 mL). The pH of the mixture was adjusted to ˜4 with HCl aqueous solution, followed by the extraction with CH₂Cl₂ (3×5 mL). The organic layers were combined, dried over MgSO₄ and concentrated in vacuo. The obtained crude product was used in the next step without further purification. LCMS calculated for C₂₇H₂₆D₄F₃N₈O₅ (M+H)⁺: m/z=607.2; found 607.2.

### Step 8. Methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

To a vial containing crude 1-(2-(difluoromethyl)-5-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-7-methyl-3H-imidazo[4,5-b]pyridin-3-yl)-3-fluorocyclobutane-1-carboxylic acid from Step 7 was added thionyl chloride (1.0 mL). The vial was heated to 50° C. for 30 min. After cooling to r.t., the reaction mixture was concentrated in vacuo to remove thionyl chloride. The crude acid chloride was dissolved in THF (2.0 mL) and added dropwise to the stirring ammonia aqueous solution (5.0 mL) at 0° C. After the addition of the acid chloride solution, the reaction mixture was extracted into CH₂Cl₂. The organic layers were combined, dried over MgSO₄ and concentrated in vacuo. The crude product was purified by prep-LCMS as the second peak of two diastereoisomers (XBridge C18 column, eluting with a gradient of 15.9→30.4% acetonitrile in water containing 0.1% TFA, at flow rate of 60 mL/min over 12 min). The desired product was collected at a retention time of 11.1 min as a single diastereomer. LCMS calculated for C₂₇H₂₇D₄F₃N₉O₄ (M+H)⁺: m/z=606.3; found 606.2.

### Example 23. Methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

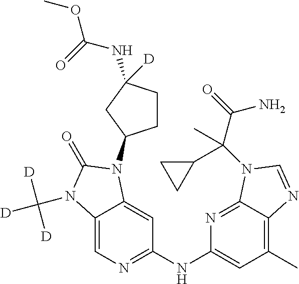 [SMILES: [2H]C([2H])([2H])n1c(=O)n([C@@H]2CC[C@@]([2H])(NC(=O)OC)C2)c2cc(Nc3cc(C)c4ncn(C(C)(C(N)=O)C5CC5)c4n3)ncc21]

Step 1. 2-((3-Amino-6-chloro-4-methylpyridin-2-yl)amino)-2-cyclopropylpropan-1-ol

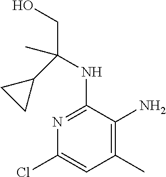 [SMILES: Cc1cc(Cl)nc(NC(C)(CO)C2CC2)c1N]

This compound was prepared according to the procedures described in Example 22, Step 1 to 2, with 2-amino-2-cyclopropylpropan-1-ol replacing (1-amino-3-fluorocyclobutyl)methanol hydrochloride in Step 1. The obtained crude product was used in the next step without further purification as a racemic mixture. LCMS calculated for C₁₂H₁₉ClN₃O (M+H)⁺: m/z=256.1; found 256.1.

### Step 2. 2-(5-Chloro-7-methyl-3H-imidazo[4,5-b]pyridin-3-yl)-2-cyclopropylpropan-1-ol

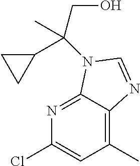 [SMILES: Cc1cc(Cl)nc2c1ncn2C(C)(CO)C1CC1]

To a vial containing 2-((3-amino-6-chloro-4-methylpyridin-2-yl)amino)-2-cyclopropylpropan-1-ol (100 mg, 0.39 mmol) in triethyl orthoformate (590 mg, 0.66 mL, 3.9 mmol) was added p-toluenesulfonic acid monohydrate (1 mg). The reaction mixture was heated at 90° C. for 30 min. The reaction mixture was cooled down to r.t. and quenched with sat. aq. NH₄Cl solution (3 mL) and extracted with CH₂Cl₂. The organic layers were combined, dried over MgSO₄ and concentrated in vacuo. The obtained crude product was used in the next step without further purification. LCMS calculated for Cl₃H₁₇ClN₃O (M+H)⁺: m/z=266.1; found 266.1.

### Step 3. Methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

This compound was prepared according to the procedures described in Example 22, Step 5 to 8, with 2-(5-chloro-7-methyl-3H-imidazo[4,5-b]pyridin-3-yl)-2-cyclopropylpropan-1-ol replacing (1-(5-chloro-2-(difluoromethyl)-7-methyl-3H-imidazo[4,5-b]pyridin-3-yl)-3-fluorocyclobutyl)methanol in Step 5. The crude product was purified by prep-LCMS (XBridge C18 column, eluting with a gradient of acetonitrile/water containing 0.1% TFA, at flow rate of 60 mL/min). The final product was obtained as a mixture of two diastereomers. LCMS calculated for C₂₇H₃₀D₄N₉O₄ (M+H)⁺: m/z=552.3; found 552.3.

### Example 24. Methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

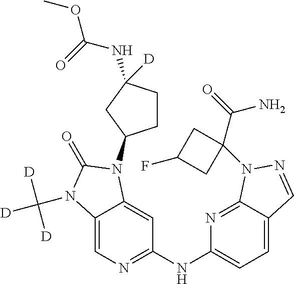 [SMILES: [2H]C([2H])([2H])n1c(=O)n([C@@H]2CC[C@@]([2H])(NC(=O)OC)C2)c2cc(Nc3ccc4cnn(C5(C(N)=O)CC(F)C5)c4n3)ncc21]

### Step 1. 3-Fluoro-1-(isopropoxycarbonyl)cyclobutane-1-carboxylic acid

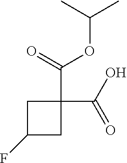

A round-bottom flask containing a solution of di-iso-propyl 3-fluorocyclobutane-1,1-dicarboxylate (16.0 g, 65.0 mmol) in iso-propanol (50 mL) was placed in an ice bath at 0° C. A solution of potassium hydroxide (3.65 g, 65.0 mmol) in iso-propanol (50 mL) and water (25 mL) was added via addition funnel to the di-ester solution at the rate of 1 drop/sec. The reaction mixture was allowed to warm up to r.t. after the addition of potassium hydroxide solution. After stirring at r.t for 16 h, the reaction mixture was concentrated in vacuo to remove iso-propanol, and then acidified with hydrochloric acid. The resulting solution was extracted with EtOAc three times. The organic phase was washed with brine, dried over MgSO₄, and concentrated in vacuo. The crude mixture was azeotroped with of toluene (30 mL) for three times to remove the remaining iso-propanol in the residue. The resulting colorless oil was used in the next step without further purification. ¹H NMR (400 MHz, CDCl₃) δ 5.32-4.98 (m, 2H), 3.06-2.66 (m, 4H), 1.36-1.22 (m, 6H).

### Step 2. Isopropyl 1-(((benzyloxy)carbonyl)amino)-3-fluorocyclobutane-1-carboxylate

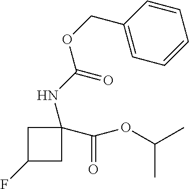 [SMILES: CC(C)OC(=O)C1(NC(=O)OCc2ccccc2)CC(F)C1]

To a vial containing 3-fluoro-1-(isopropoxycarbonyl)cyclobutane-1-carboxylic acid (1.0 g, 4.9 mmol) in toluene (20 mL) was added triethylamine (0.78 mL, 5.6 mmol) and diphenylphosphoryl azide (1.16 mL, 5.40 mmol). After heating at 50° C. for 20 min, the reaction mixture was cooled down to r.t., followed by the addition of phenylmethanol (0.69 g, 6.4 mmol) and DMAP (5 mg). Then the reaction mixture was heated to 111° C. for 16 h (note: nitrogen gas needs to be released). The reaction mixture was cooled down to r.t and concentrated in vacuo, purified by Teledyne ISCO CombiFlash to give a mixture of two isomers. The mixture was further separated by chiral SFC (CHIRALPAK IG, 30 mm×250 mm, 5 um column, eluting with 20% methanol in CO₂, at flow rate of 100 mL/min over 6 min). Retention time was 3.5 min for peak 1 and 4.7 min for peak 2. The desired isomer was collected as the second peak at 4.7 min as a single compound. LCMS calculated for C₁₆H₂₁FNO₄(M+H)⁺: m/z=310.1; found: 310.1.

### Step 3. Isopropyl 1-amino-3-fluorocyclobutane-1-carboxylate

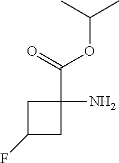

To a vial containing isopropyl 1-(((benzyloxy)carbonyl)amino)-3-fluorocyclobutane-1-carboxylate (1.52 g, 4.9 mmol, Step 2) in MeOH (20 mL) was added Pd/C (30 mg, 10% w/w). The vial was purged with hydrogen gas for 5 min and then stirred for 16 h under an atmosphere of hydrogen at 40° C. After cooling to room temperature, the reaction mixture was filtered through Celite and washed with CH₂Cl₂, followed by concentration of the filtrate in vacuo. The crude product was used directly in the next step without further purification. LCMS calculated for C₈H₁₅FNO₂ (M+H)⁺: m/z=176.1; found: 176.1

### Step 4. Isopropyl 1-((6-bromo-3-cyanopyridin-2-yl)amino)-3-fluorocyclobutane-1-carboxylate

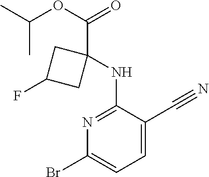 [SMILES: CC(C)OC(=O)C1(Nc2nc(Br)ccc2C#N)CC(F)C1]

To a vial containing 6-bromo-2-fluoronicotinonitrile (500 mg, 2.49 mmol) and isopropyl 1-amino-3-fluorocyclobutane-1-carboxylate (440 mg, 2.49 mmol) in DMSO (830 μL) was added DIPEA (520 μL, 3.00 mmol). After heating at 65° C. for 16 h, the reaction mixture was directly purified by Biotage Isolera to give the desired product as light-yellow solid. LCMS calculated for C₁₄H₁₆BrFN₃O₂(M+H)⁺: m/z=356.0/358.0; found 356.0/358.0.

### Step 5. Isopropyl 1-(3-amino-6-bromo-1H-pyrazolo[3,4-b]pyridin-1-yl)-3-fluorocyclobutane-1-carboxylate

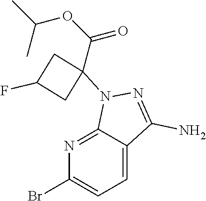 [SMILES: CC(C)OC(=O)C1(n2nc(N)c3ccc(Br)nc32)CC(F)C1]

To a vial containing isopropyl 1-((6-bromo-3-cyanopyridin-2-yl)amino)-3-fluorocyclobutane-1-carboxylate (750 mg, 2.11 mmol) in THF (2.8 mL) and DMF (5.6 mL) at 0° C. was slowly added LHMDS (2.7 mL, 2.74 mmol, 1.0 M in THF). After stirring at 0° C. for 10 min, (aminooxy)diphenylphosphine oxide (590 mg, 2.53 mmol) was added to the reaction mixture. The reaction was allowed to warm up to r.t. with monitoring of stirring rate. After stirring at r.t. for 1 h, the reaction mixture was treated with 1 M HCl solution (5 mL) and stirred for 10 min. The reaction mixture was neutralized with sat. NaHCO₃ solution, followed by extraction with diethyl ether (15 mL) for 3 times. The organic phase was combined and washed with brine, dried over MgSO₄ and concentrated in vacuo. The crude material was purified by Biotage Isolera to give the desired product as light-yellow solid. LCMS calculated for C₁₄H₁₇BrFN₄O₂(M+H)⁺: m/z=371.0/373.0; found 371.1/373.0.

### Step 6. Isopropyl 1-(6-bromo-1H-pyrazolo[3,4-b]pyridin-1-yl)-3-fluorocyclobutane-1-carboxylate

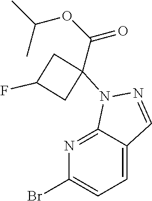 [SMILES: CC(C)OC(=O)C1(n2ncc3ccc(Br)nc32)CC(F)C1]

To a vial containing isopropyl 1-(3-amino-6-bromo-1H-pyrazolo[3,4-b]pyridin-1-yl)-3-fluorocyclobutane-1-carboxylate (700 mg, 1.89 mmol) in THF (2.8 mL) was slowly added tert-butyl nitrite (1.11 mL, 8.42 mmol). After heating at 65° C. for 1.5 h, the reaction mixture was concentrated in vacuo. The crude material was purified by Biotage Isolera to give the desired product as light-yellow solid. LCMS calculated for C₁₄H₁₆BrFN₃O₂(M+H)⁺: m/z=356.0/358.0; found 356.0/358.0.

### Step 7. Isopropyl 3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

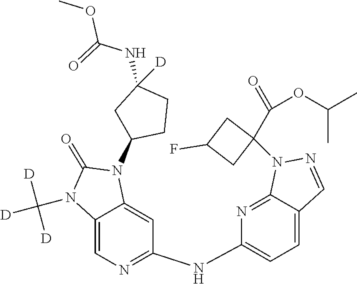 [SMILES: [2H]C([2H])([2H])n1c(=O)n([C@@H]2CC[C@@]([2H])(NC(=O)OC)C2)c2cc(Nc3ccc4cnn(C5(C(=O)OC(C)C)CC(F)C5)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (33 mg, 0.11 mmol, Intermediate A), isopropyl 1-(6-bromo-1H-pyrazolo[3,4-b]pyridin-1-yl)-3-fluorocyclobutane-1-carboxylate (38 mg, 0.11 mmol), tris(dibenzylideneacetone)-dipalladium(0) (12 mg, 0.012 mmol), 4,5-bis(diphenylphosphino)-9,9-dimethylxanthene (15 mg, 0.025 mmol), and cesium carbonate (70 mg, 0.21 mmol) was evacuated and backfilled with nitrogen three times, followed by the addition of 1,4-dioxane (0.5 mL). The vial was sealed and heated to 100° C. for 2 h. After cooling to r.t., the mixture was filtered and concentrated in vacuo. The crude material was purified by Biotage Isolera to give the desired product as off-white solid. LCMS calculated for C₂₈H₃₀D₄FN₈O₅(M+H)⁺: m/z=585.3; found 585.3.

### Step 8. Methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a vial containing isopropyl 3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (20 mg, 0.034 mmol) in THF/MeOH/water (1.5 mL, v/v/v=1/1/1) was added LiOH (7 mg, 0.3 mmol). After heating at 50° C. for 15 min, the reaction mixture was cooled to r.t. and diluted with CH₂Cl₂ (5 mL). The pH of the mixture was adjusted to ˜4 using HCl aqueous solution, followed by extraction with CH₂Cl₂ (3×5 mL). The organic layers were combined, dried over MgSO₄ and concentrated in vacuo. To a vial containing the obtained crude acid was added thionyl chloride (1.0 mL). The reaction mixture was heated to 50° C. for 30 min. After cooling to r.t., the reaction mixture was concentrated in vacuo to remove thionyl chloride. The crude acid chloride was dissolved in THF (2.0 mL) and added dropwise to the stirring ammonia aqueous solution (5.0 mL) at 0° C. After the addition of the acid chloride solution, the reaction mixture was extracted into CH₂Cl₂. The organic layers were combined, dried over MgSO₄ and concentrated in vacuo. The crude product was purified by prep-LCMS (XBridge C18 column, eluting with a gradient of acetonitrile/water containing 0.1% TFA, at flow rate of 60 mL/min). The final product was obtained as a single diastereomer. LCMS calculated for C₂₅H₂₅D₄N₉O₄(M+H)⁺: m/z=542.2; found 542.2. ¹H NMR (600 MHz, DMSO) δ 11.42 (brs, 1H), 8.27 (s, 1H), 8.25 (d, J=9.0 Hz, 1H), 8.19 (s, 1H), 7.58 (s, 1H), 7.53 (brs, 1H), 7.34 (s, 1H), 7.28 (brs, 1H), 7.02 (d, J=8.7 Hz, 1H), 5.32-5.14 (m, 1H), 5.01 (p, J=8.5 Hz, 1H), 3.55 (s, 3H), 3.47-3.40 (m, 2H), 3.12-3.02 (m, 2H), 2.34 (dd, J=13.6, 7.5 Hz, 1H), 2.25-2.13 (m, 2H), 2.10-2.02 (m, 1H), 1.97 (dd, J=13.6, 9.6 Hz, 1H), 1.66 (dt, J=12.4, 8.0 Hz, 1H).

### Example 25. Methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate and Methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

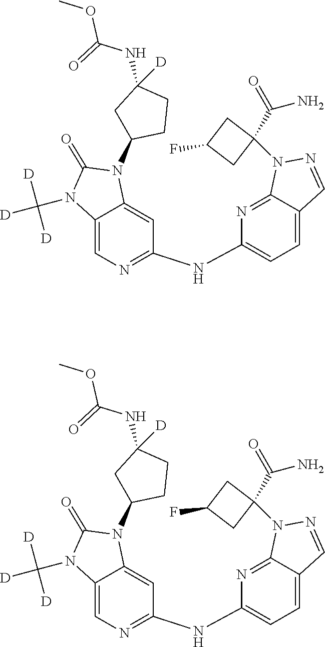 [SMILES: [2H]C([2H])([2H])n1c(=O)n([C@@H]2CC[C@@]([2H])(NC(=O)OC)C2)c2cc(Nc3ccc4cnn([C@]5(C(N)=O)C[C@@H](F)C5)c4n3)ncc21 ; [2H]C([2H])([2H])n1c(=O)n([C@@H]2CC[C@@]([2H])(NC(=O)OC)C2)c2cc(Nc3ccc4cnn([C@]5(C(N)=O)C[C@H](F)C5)c4n3)ncc21]

### Step 1. Methyl 1-((6-bromo-3-cyanopyridin-2-yl)amino)-3-fluorocyclobutane-1-carboxylate

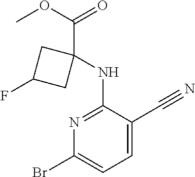 [SMILES: COC(=O)C1(Nc2nc(Br)ccc2C#N)CC(F)C1]

To a vial containing 6-bromo-2-fluoronicotinonitrile (300 mg, 1.49 mmol) and methyl 1-amino-3-fluorocyclobutane-1-carboxylate (242 mg, 1.64 mmol) in DMSO (500 μL) was added DIPEA (340 μL, 1.94 mmol). After heating at 65° C. for 16 h, the reaction mixture was directly purified by Biotage Isolera to give the desired product as light-yellow solid. LCMS calculated for C₁₂H₁₂BrFN₃O₂(M+H)⁺: m/z=328.0/330.0; found 328.0/330.0.

### Step 2. Methyl (1r,3r)-1-(3-amino-6-bromo-1H-pyrazolo[3,4-b]pyridin-1-yl)-3-fluorocyclobutane-1-carboxylate and Methyl (1s,3s)-1-(3-amino-6-bromo-1H-pyrazolo[3,4-b]pyridin-1-yl)-3-fluorocyclobutane-1-carboxylate

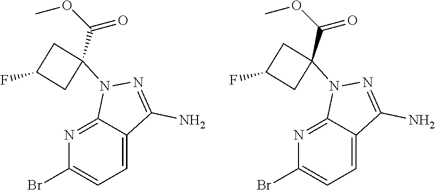 [SMILES: COC(=O)[C@]1(n2nc(N)c3ccc(Br)nc32)C[C@H](F)C1 ; COC(=O)[C@]1(n2nc(N)c3ccc(Br)nc32)C[C@@H](F)C1]

To a vial containing methyl 1-((6-bromo-3-cyanopyridin-2-yl)amino)-3-fluorocyclobutane-1-carboxylate (100 mg, 0.30 mmol) in THF (0.4 mL) and DMF (0.8 mL) at 0° C. was slowly added LHMDS (0.40 mL, 0.40 mmol, 1.0 M in THF). After stirring at 0° C. for 10 min, (aminooxy)diphenylphosphine oxide (71 mg, 0.30 mmol) was added to the reaction mixture. The reaction was allowed to warm up to r.t. with monitoring of stirring rate. After stirring at r.t. for 1 h, the reaction mixture was treated with 1 M HCl solution (2 mL) and stirred for 10 min. The reaction mixture was neutralized with sat. NaHCO₃ solution, followed by extraction with diethyl ether (5 mL) for 3 times. The organic phase was combined and washed with brine, dried over MgSO₄ and concentrated in vacuo. The crude material was purified by Biotage Isolera to give the title compound as a mixture of two diastereomers. The mixture was further separated by chiral SFC (Phenomenex-Amylose-1, 30 mm×250 mm, 5 um column, eluting with 20% methanol in CO₂, at flow rate of 125 mL/min over 5.3 min). The first peak (Peak 1) was collected at 3.1 min as a single diastereomer. The second peak (Peak 2) was collected at 3.42 min as a single diastereomer. Both peaks were individually carried forward through next steps.
Peak 1: LCMS calculated for C₁₂H₁₃BrFN₄O₂(M+H)⁺: m/z=343.0/345.0; found: 343.0/345.0.
Peak 2: LCMS calculated for C₁₂H₁₂BrFN₄O₂(M+H)⁺: m/z=343.0/345.0; found 343.1/345.0.

### Step 3. Methyl (1r,3r)-1-(6-bromo-1H-pyrazolo[3,4-b]pyridin-1-yl)-3-fluorocyclobutane-1-carboxylate and Methyl (1s,3s)-1-(6-bromo-1H-pyrazolo[3,4-b]pyridin-1-yl)-3-fluorocyclobutane-1-carboxylate

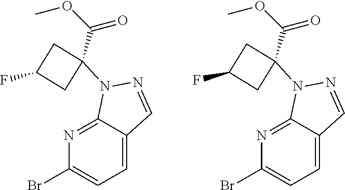 [SMILES: COC(=O)[C@]1(n2ncc3ccc(Br)nc32)C[C@H](F)C1 ; COC(=O)[C@]1(n2ncc3ccc(Br)nc32)C[C@@H](F)C1]

To a vial containing methyl 1-(3-amino-6-bromo-1H-pyrazolo[3,4-b]pyridin-1-yl)-3-fluorocyclobutane-1-carboxylate (40 mg, 0.12 mmol, isomer of Peak 1 from Step 2) in THF (0.4 mL) was slowly added tert-butyl nitrite (70 μL, 0.5 mmol). After heating at 65° C. for 1.5 h, the reaction mixture was concentrated in vacuo. The crude material was purified by Biotage Isolera to give the desired product (Intermediate 25-3-1) as light-yellow solid. LCMS calculated for C₁₂H₁₂BrFN₃O₂(M+H)⁺: m/z=328.0/330.0; found 328.1/330.0. The other isomer (Intermediate 25-3-2) was also prepared in the same manner starting with the isomer of Peak 2 in Step 2. LCMS calculated for C₁₂H₁₂BrFN₃O₂(M+H)⁺: m/z=328.0/330.0; found 328.0/330.0 (for both Intermediates 25-3-1 and 25-3-2).

### Step 4. Methyl (1r,3R)-3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate and Methyl (1s, 3S)-3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

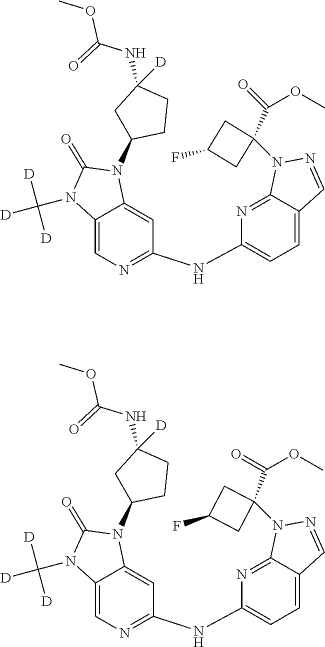 [SMILES: [2H]C([2H])([2H])n1c(=O)n([C@@H]2CC[C@@]([2H])(NC(=O)OC)C2)c2cc(Nc3ccc4cnn([C@]5(C(=O)OC)C[C@@H](F)C5)c4n3)ncc21 ; [2H]C([2H])([2H])n1c(=O)n([C@@H]2CC[C@@]([2H])(NC(=O)OC)C2)c2cc(Nc3ccc4cnn([C@]5(C(=O)OC)C[C@H](F)C5)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (28 mg, 0.09 mmol, Intermediate A), methyl 1-(6-bromo-1H-pyrazolo[3,4-b]pyridin-1-yl)-3-fluorocyclobutane-1-carboxylate (30 mg, 0.09 mmol, Intermediate 25-3-1 of Step 3), tris(dibenzylideneacetone)-dipalladium(0) (9.0 mg, 0.010 mmol), 4,5-bis(diphenylphosphino)-9,9-dimethylxanthene (11.6 mg, 0.020 mmol), and cesium carbonate (60 mg, 0.18 mmol) was evacuated and backfilled with nitrogen three times, followed by the addition of 1,4-dioxane (0.5 mL). The vial was sealed and heated to 100° C. for 2 h. After cooling to r.t., the mixture was filtered and concentrated in vacuo. The crude material was purified by Biotage Isolera to give the desired product (Intermediate 25-4-1) as off-white solid. LCMS calculated for C₂₆H₂₆D₄FN₈O₅(M+H)⁺: m/z=557.3; found 557.3. The other isomer was also prepared in the same manner starting with Intermediate 25-3-2 of Step 3. LCMS calculated for C₂₆H₂₆D₄FN₈O₅(M+H)⁺: m/z=557.3; found 557.3.

### Step 5. Methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate and Methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a vial containing methyl 3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (20 mg, 0.036 mmol, Intermediate 25-4-1 of Step 4) in THF/MeOH/water (1.5 mL, v/v/v=1/1/1) was added LiOH (7 mg, 0.3 mmol). After heating at 50° C. for 15 min, the reaction mixture was cooled to r.t. and diluted with CH₂Cl₂ (5 mL). The pH of the mixture was adjusted to ˜4 using HCl aqueous solution, followed by extraction with CH₂Cl₂ (3×5 mL). The organic layers were combined, dried over MgSO₄ and concentrated in vacuo. To a vial containing the obtained crude acid was added thionyl chloride (1.0 mL). The reaction mixture was heated to 50° C. for 30 min. After cooling to r.t., the reaction mixture was concentrated in vacuo to remove thionyl chloride. The crude acid chloride was dissolved in THF (2.0 mL) and added dropwise to the stirring ammonia aqueous solution (5.0 mL) at 0° C. After the addition of the acid chloride solution, the reaction mixture was extracted into CH₂Cl₂. The organic layers were combined, dried over MgSO₄ and concentrated in vacuo. The crude product was purified by prep-LCMS (XBridge C18 column, eluting with a gradient of acetonitrile/water containing 0.1% TFA, at flow rate of 60 mL/min) resulting in single isomer Product 1. The other isomer (Product 2) was also prepared in the same manner starting with Intermediate 25-4-2 of Step 4.
Product 1: LCMS calculated for C₂₅H₂₅D₄FN₉O₄(M+H)⁺: m/z=542.3; found 542.3.
Product 2: LCMS calculated for C₂₅H₂₅D₄FN₉O₄(M+H)⁺: m/z=542.3; found 542.3.

### Example 26. Methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate and Methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

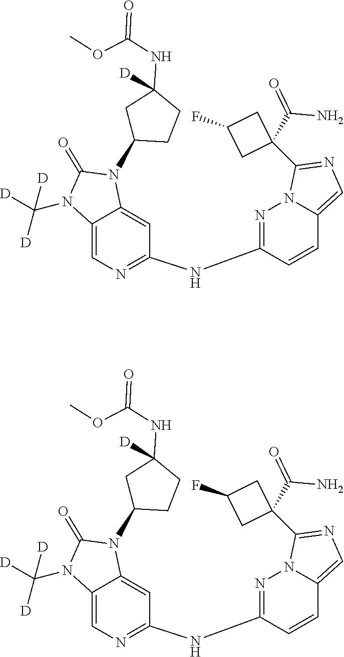 [SMILES: [2H]C([2H])([2H])n1c(=O)n([C@@H]2CC[C@@]([2H])(NC(=O)OC)C2)c2cc(Nc3ccc4cnc([C@]5(C(N)=O)C[C@@H](F)C5)n4n3)ncc21 ; [2H]C([2H])([2H])n1c(=O)n([C@@H]2CC[C@@]([2H])(NC(=O)OC)C2)c2cc(Nc3ccc4cnc([C@]5(C(N)=O)C[C@H](F)C5)n4n3)ncc21]

### Step 1. Isopropyl (1s,3s)-1-(2-chloroimidazo[1,5-b]pyridazin-7-yl)-3-fluorocyclobutane-1-carboxylate 1-(2-chloroimidazo[1,5-b]pyridazin-7-yl)-3-fluorocyclobutane-1-carboxylate and Isopropyl (1r,3r)-1-(2-chloroimidazo[1,5-b]pyridazin-7-yl)-3-fluorocyclobutane-1-carboxylate

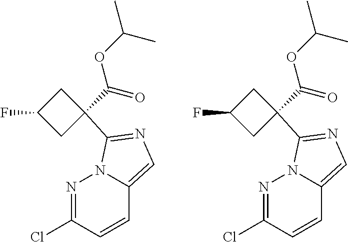 [SMILES: CC(C)OC(=O)[C@]1(c2ncc3ccc(Cl)nn32)C[C@H](F)C1 ; CC(C)OC(=O)[C@]1(c2ncc3ccc(Cl)nn32)C[C@@H](F)C1]

The title compound was prepared according to the procedures described in Example 5, Steps 1-2. After SFC chiral purification, the compounds of both peaks were individually carried forward through next steps. Retention time was 3.5 min for Peak 1 and 4.9 min for Peak 2.
Peak 1: LCMS calculated for C₁₄H₁₆ClFN₃O₂(M+H)⁺: m/z=312.1; found 312.1.
Peak 2: LCMS calculated for C₁₄H₁₆ClFN₃O₂(M+H)⁺: m/z=312.1; found 312.1.

### Step 2. Methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate and Methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compounds were prepared according to the procedures described in Example 5, Steps 3-5, with isopropyl 1-(2-chloroimidazo[1,5-b]pyridazin-7-yl)-3-fluorocyclobutane-1-carboxylate (peak 1 and peak 2 individually) replacing isopropyl 1-(2-chloroimidazo[1,5-b]pyridazin-7-yl)-3-fluorocyclobutane-1-carboxylate in Step 3. Each desired product (Product 1 obtained using isomer of Peak 1 in Step 1 and Product 2 obtained using isomer of Peak 2 in Step 1) was obtained as a single diastereomer.
Product 1: LCMS calculated for C₂₅H₂₅D₄FN₉O₄(M+H)⁺: m/z=542.3; found 542.3.
Product 2: LCMS calculated for C₂₅H₂₅D₄FN₉O₄(M+H)⁺: m/z=542.3; found 542.3.

### Example 27. Methyl ((1R,3R)-3-(6-((1-((1S,2S)-1-carbamoyl-2-methylcyclopropyl)-3-ethyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

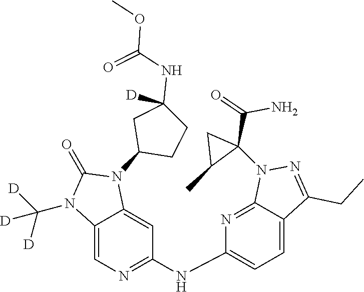 [SMILES: [2H]C([2H])([2H])n1c(=O)n([C@@H]2CC[C@@]([2H])(NC(=O)OC)C2)c2cc(Nc3ccc4c(CC)nn([C@@]5(C(N)=O)C[C@@H]5C)c4n3)ncc21]

The title compound was prepared according to the procedures described in Example 55, Step 10-11, with ethylzinc chloride replacing methylzinc chloride. LCMS calculated for: C₂₇H₃₀D₄N₉O₄ (M+H)⁺: m/z=552.3; found 552.3. ¹H NMR (500 MHz, DMSO) δ 10.76 (s, 1H), 8.25 (s, 1H), 8.20 (d, J=8.6 Hz, 1H), 7.90 (s, 1H), 7.36 (s, 1H), 7.32 (s, 1H), 6.90 (s, 1H), 6.64 (s, 1H), 4.97-4.90 (m, 1H), 3.54 (s, 3H), 2.80 (m, 2H), 2.37 (m, 1H), 2.26-2.01 (m, 4H), 1.95 (m, 1H), 1.77 (m, 1H), 1.68-1.63 (m, 1H), 1.43 (m, 1H), 1.33 (m, 1H), 1.25 (m, 5H).

### Example 28. Methyl ((1R,3R)-3-(6-((1-((1S,2S)-1-carbamoyl-2-methylcyclopropyl)-3-(2-methoxypyridin-3-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

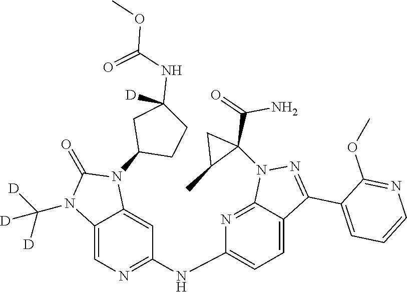 [SMILES: [2H]C([2H])([2H])n1c(=O)n([C@@H]2CC[C@@]([2H])(NC(=O)OC)C2)c2cc(Nc3ccc4c(-c5cccnc5OC)nn([C@@]5(C(N)=O)C[C@@H]5C)c4n3)ncc21]

### Step 1. Benzyl (1S,2S)-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(2-methoxypyridin-3-yl)-1H-pyrazolo[3,4-b]pyridin-1-yl)-2-methylcyclopropane-1-carboxylate

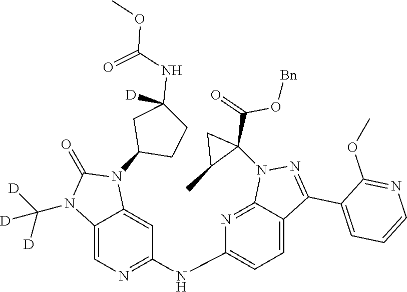 [SMILES: [2H]C([2H])([2H])n1c(=O)n([C@@H]2CC[C@@]([2H])(NC(=O)OC)C2)c2cc(Nc3ccc4c(-c5cccnc5OC)nn([C@@]5(C(=O)OCc6ccccc6)C[C@@H]5C)c4n3)ncc21]

The vial containing benzyl (1S,2S)-1-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (220 mg, 0.297 mmol), (2-methoxypyridin-3-yl)boronic acid (55 mg, 0.356 mmol), tetrakis(triphenylphosphine)palladium(0) (69 mg, 0.059 mmol) and cesium carbonate (290 mg, 0.891 mmol) was evacuated and backfilled with N₂ three times. Dioxane (3.0 mL) and water (0.8 mL) were then added. The mixture was evacuated and backfilled with N₂ and heated to 75° C. for 2 h. The mixture was cooled to r.t. and extracted with EtOAc twice. The combined organic layers were washed by brine, dried over Na₂SO₄, filtered, and concentrated in vacuo. The crude material was purified by Biotage Isolera to give the desired product. LCMS calculated for C₃₈H₃₆D₄N₉O₆(M+H)⁺: m/z=722.3; found 722.3.

### Step 2. Methyl ((1R,3R)-3-(6-((1-((1S,2S)-1-carbamoyl-2-methylcyclopropyl)-3-(2-methoxypyridin-3-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 55, Step 11, with benzyl (1S,2S)-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(2-methoxypyridin-3-yl)-1H-pyrazolo[3,4-b]pyridin-1-yl)-2-methylcyclopropane-1-carboxylate replacing benzyl (1S,2S)-1-(3-(bis(tert-butoxycarbonyl)amino)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate. LCMS calculated for C₃₁H₃₁D₄N₁₀O₅(M+H)⁺: m/z=631.3; found 631.3. ¹H NMR (600 MHz, DMSO) δ 11.30 (s, 1H), 8.25-8.30 (m, 3H), 8.18 (m, 1H), 7.66 (s, 1H), 7.45 (s, 1H), 7.33 (s, 1H), 7.20 (m, 1H), 7.15 (m, 2H), 4.95 (m, 1H), 3.95 (s, 3H), 3.55 (s, 3H), 2.35 (m, 1H), 2.20-2.25 (m, 3H), 2.07 (m, 1H), 1.95 (m, 1H), 1.77 (m, 1H), 1.63 (m, 1H), 1.50 (m, 1H), 1.32 (m, 3H).

### Example 29. Methyl ((1R,3R)-3-(6-((1-((1S,2S)-1-carbamoyl-2-methylcyclopropyl)-3-(4-methoxypyridin-3-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

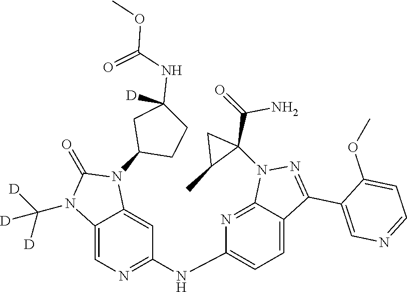 [SMILES: [2H]C([2H])([2H])n1c(=O)n([C@@H]2CC[C@@]([2H])(NC(=O)OC)C2)c2cc(Nc3ccc4c(-c5cnccc5OC)nn([C@@]5(C(N)=O)C[C@@H]5C)c4n3)ncc21]

The title compound was prepared according to the procedures described in Example 28, Step 1-2, with 4-methoxy-3-(4,4,5,5-tetramethyl-1,3,2-dioxaborolan-2-yl)pyridine replacing (2-methoxypyridin-3-yl)boronic acid in Step 1. LCMS calculated for C₃₁H₃₁D₄N₁₀O₅(M+H)⁺: m/z=631.3; found 631.3. ¹H NMR (600 MHz, DMSO) δ 10.85 (s, 1H), 9.10 (s, 1H), 8.85 (m, 1H), 8.22 (s, 1H), 8.15 (m, 1H), 8.10 (s, 1H), 7.80 (m, 1H), 7.50 (s, 1H), 7.25 (s, 1H), 7.15 (m, 1H), 6.95 (s, 1H), 4.95 (m, 1H), 4.20 (s, 3H), 3.51 (s, 3H), 2.40 (m, 1H), 2.10-2.25 (m, 4H), 1.95 (m, 1H), 1.80 (m, 1H), 1.60 (m, 1H), 1.50 (m, 1H), 1.32 (m, 3H).

### Example 30. Methyl ((1R,3R)-3-(6-((1-((1S,2R)-1-carbamoyl-2-isopropylcyclopropyl)-3-((methoxycarbonyl)amino)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

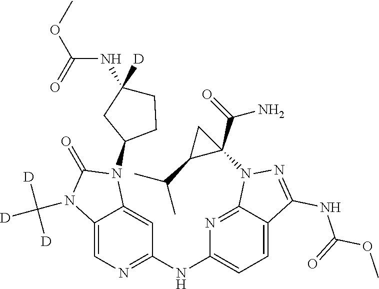 [SMILES: [2H]C([2H])([2H])n1c(=O)n([C@@H]2CC[C@@]([2H])(NC(=O)OC)C2)c2cc(Nc3ccc4c(NC(=O)OC)nn([C@@]5(C(N)=O)C[C@@H]5C(C)C)c4n3)ncc21]

The title compound was prepared according to the procedures described in Example 54, Steps 1-8 followed by the procedure described in step 11 with rac-methyl (1R,2S)-1-amino-2-(propan-2-yl)cyclopropane-1-carboxylate hydrochloride replacing benzyl (1S,2S)-1-amino-2-methylcyclopropane-1-carboxylate in Step 2, and methyl chloroformate (3.0 eq.), DIPEA (6.0 eq.) in CH₂Cl₂ was used in step 7 for carbamate protection instead of Boc-protection. Final purification was done by prep-LCMS (XBridge C18 column, eluting with acetonitrile/aqueous 0.1% NH₄OH, at flow rate of 60 mL/min). The desired diastereomer was collected at a retention time of 8.5 min. LCMS calculated for C₂₉H₃₃D₄N₁₀O₆ (M+H)⁺: m/z=625.3; found 625.5. ¹H NMR (500 MHz, DMSO) δ 10.14 (s, 1H), 9.98 (s, 1H), 8.47 (s, 1H), 8.16-8.00 (m, 2H), 7.38 (s, 1H), 7.22 (s, 1H), 6.98 (d, J=8.9 Hz, 1H), 6.47 (s, 1H), 5.03-4.80 (m, 1H), 3.69 (s, 3H), 3.51 (s, 3H), 2.50-2.42 (m, 1H), 2.28-2.18 (m, 1H), 2.17-2.05 (m, 2H), 1.94 (dd, J=13.3, 9.1 Hz, 1H), 1.86-1.72 (m, 3H), 1.69-1.57 (m, 1H), 1.48-1.36 (m, 1H), 1.07 (d, J=5.3 Hz, 3H), 1.01 (d, J=5.7 Hz, 3H).

### Example 31. Methyl ((1R,3R)-3-(6-((1-((1S,2S)-1-carbamoyl-2-(trifluoromethyl)cyclopropyl)-3-((methoxycarbonyl)amino)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

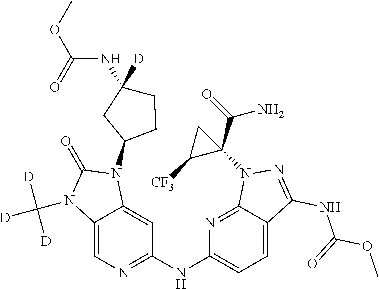 [SMILES: [2H]C([2H])([2H])n1c(=O)n([C@@H]2CC[C@@]([2H])(NC(=O)OC)C2)c2cc(Nc3ccc4c(NC(=O)OC)nn([C@@]5(C(N)=O)C[C@@H]5C(F)(F)F)c4n3)ncc21]

The title compound was prepared according to the procedures described in Example 30 with rac-methyl (1R,2R)-1-amino-2-(trifluoromethyl)cyclopropane-1-carboxylate hydrochloride replacing rac-methyl (1R,2S)-1-amino-2-(propan-2-yl)cyclopropane-1-carboxylate hydrochloride, and final purification by prep-LCMS (XBridge C18 column, eluting with acetonitrile/aqueous 0.1% NH₄OH, at flow rate of 60 mL/min). The desired diastereomer was collected at a retention time of 8.8 min. LCMS calculated for C₂₇H₂₆D₄F₃N₁₀O₆ (M+H)⁺: m/z=651.3; found 651.4. ¹H NMR (500 MHz, DMSO) δ 10.29 (s, 1H), 9.99 (s, 1H), 8.21 (s, 1H), 8.14 (d, J=8.9 Hz, 1H), 8.06 (s, 1H), 7.57 (s, 1H), 7.14-7.00 (m, 2H), 6.85 (s, 1H), 5.01-4.86 (m, 1H), 3.70 (s, 3H), 3.51 (s, 3H), 2.99-2.87 (m, 1H), 2.44-2.33 (m, 2H), 2.24-1.97 (m, 4H), 1.89 (dd, J=13.3, 9.5 Hz, 1H), 1.62-1.52 (m, 1H).

### Example 32. Methyl ((1R,3R)-3-(6-((1-((1S,2R)-1-carbamoyl-2-isopropylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

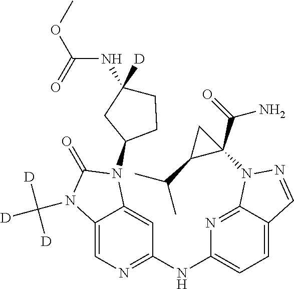 [SMILES: [2H]C([2H])([2H])n1c(=O)n([C@@H]2CC[C@@]([2H])(NC(=O)OC)C2)c2cc(Nc3ccc4cnn([C@@]5(C(N)=O)C[C@@H]5C(C)C)c4n3)ncc21]

The title compound was prepared according to the procedures described in Example 16 with rac-methyl (1R,2S)-1-amino-2-(propan-2-yl)cyclopropane-1-carboxylate hydrochloride replacing rac-methyl (1R,2R)-1-amino-2-methylcyclopropane-1-carboxylate hydrochloride and final purification was done by prep-LCMS (XBridge C18 column, eluting with acetonitrile/aqueous 0.1% NH₄OH, at flow rate of 60 mL/min). The desired diastereomer was collected at a retention time of 8.6 min. LCMS calculated for C₂₇H₃₀D₄N₉O₄ (M+H)⁺: m/z=552.3; found 552.4. ¹H NMR (600 MHz, DMSO) δ 10.01 (s, 1H), 8.53 (s, 1H), 8.06 (s, 1H), 7.98-7.91 (m, 2H), 7.38 (s, 1H), 7.24 (s, 1H), 7.07 (d, J=8.7 Hz, 1H), 6.37 (s, 1H), 5.00-4.90 (m, 1H), 3.52 (s, 3H), 2.48-2.45 (m, 1H), 2.27-2.20 (m, 1H), 2.16-2.07 (m, 2H), 2.00-1.77 (m, 4H), 1.68-1.60 (m, 1H), 1.41 (dd, J=9.3, 4.5 Hz, 1H), 1.11 (d, J=6.4 Hz, 3H), 1.02 (d, J=6.5 Hz, 3H).

### Example 33. Methyl ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

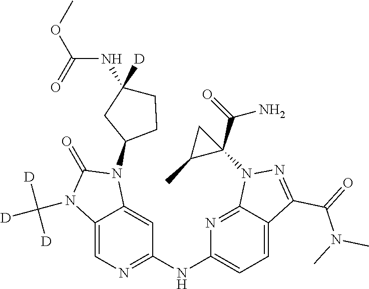 [SMILES: [2H]C([2H])([2H])n1c(=O)n([C@@H]2CC[C@@]([2H])(NC(=O)OC)C2)c2cc(Nc3ccc4c(C(=O)N(C)C)nn([C@@]5(C(N)=O)C[C@@H]5C)c4n3)ncc21]

### Step 1. Benzyl (1S,2S)-1-(6-bromo-3-iodo-1H-pyrazolo[3,4-b]pyridin-1-yl)-2-methylcyclopropane-1-carboxylate

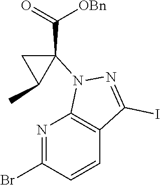 [SMILES: C[C@H]1C[C@]1(C(=O)OCc1ccccc1)n1nc(I)c2ccc(Br)nc21]

The title compound was prepared according to the procedures described in Example 54, steps 1-6 followed by the procedure described in step 9 to convert the amino group into iodo. LCMS calculated for C₁₈H₁₆BrIN₃O₂ (M+H)⁺: m/z=511.9; found 512.0.

### Step 2. Benzyl (1S,2S)-1-(6-bromo-3-formyl-1H-pyrazolo[3,4-b]pyridin-1-yl)-2-methylcyclopropane-1-carboxylate

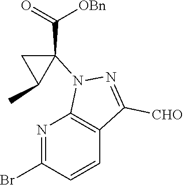 [SMILES: C[C@H]1C[C@]1(C(=O)OCc1ccccc1)n1nc(C=O)c2ccc(Br)nc21]

The title compound was prepared according to the procedures described in Example 17, steps 3-4. LCMS calculated for C₁₉H₁₇BrN₃O₃(M+H)⁺: m/z=414.0; found 414.0.

### Step 3. Benzyl (1S,2S)-1-(3-formyl-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

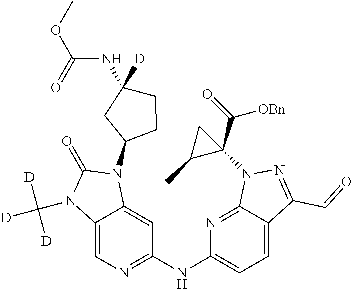 [SMILES: [2H]C([2H])([2H])n1c(=O)n([C@@H]2CC[C@@]([2H])(NC(=O)OC)C2)c2cc(Nc3ccc4c(C=O)nn([C@@]5(C(=O)OCc6ccccc6)C[C@@H]5C)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (1.6 g, 5.2 mmol), benzyl (1S,2S)-1-(6-bromo-3-formyl-1H-pyrazolo[3,4-b]pyridin-1-yl)-2-methylcyclopropane-1-carboxylate (1.9 g, 4.7 mmol), tris(dibenzylideneacetone)-dipalladium(0) (0.82 g, 0.9 mmol), 4,5-bis(diphenylphosphino)-9,9-dimethylxanthene (1.0 g, 1.8 mmol), and cesium carbonate (4.6 g, 14.2 mmol) was evacuated and backfilled with nitrogen three times, followed by the addition of 1,4-dioxane (40 mL). The vial was sealed and heated to 100° C. for 2 h. After cooling to r.t., the mixture was filtered and concentrated in vacuo. The crude material was purified by Biotage Isolera to give the corresponding aldehyde intermediate. LCMS calculated for C₃₃H₃₁D₄N₈O₆(M+H)⁺: m/z=643.2; found 643.3.

### Step 4. 1-((1S,2S)-1-((Benzyloxy)carbonyl)-2-methylcyclopropyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridine-3-carboxylic acid

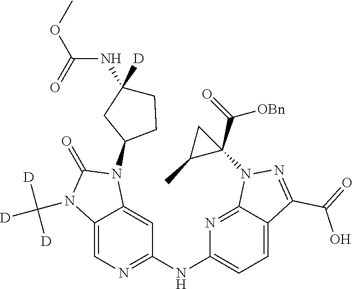 [SMILES: [2H]C([2H])([2H])n1c(=O)n([C@@H]2CC[C@@]([2H])(NC(=O)OC)C2)c2cc(Nc3ccc4c(C(=O)O)nn([C@@]5(C(=O)OCc6ccccc6)C[C@@H]5C)c4n3)ncc21]

To a solution of the aldehyde intermediate (1.9 g, 3.0 mmol) in tBuOH (10 mL) was added 2-methyl-2-butene (3.0 mL, 29.3 mmol) followed by the addition of a mixture of sodium chlorite (2.8 g, 30.7 mmol) and NaH₂PO₄H₂O (2.2 g, 18.4 mmol) in water (10 mL) and the reaction mixture was stirred for 1 h at room temperature. The reaction mixture was extracted with ethyl acetate, washed with 1 N HCl, water and brine. The organic layer was dried over MgSO₄, filtered and concentrated in vacuo. The crude acid intermediate was used for the next step without further purification. LCMS calculated for C₃₃H₃₁D₄N₈O₇(M+H)⁺: m/z=659.3; found 659.4.

### Step 5. Benzyl (1S,2S)-1-(3-(dimethylcarbamoyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

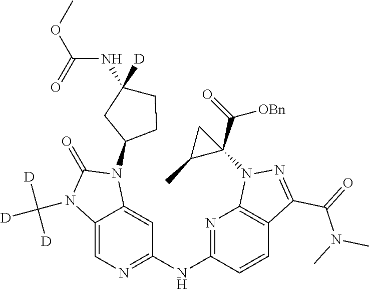 [SMILES: [2H]C([2H])([2H])n1c(=O)n([C@@H]2CC[C@@]([2H])(NC(=O)OC)C2)c2cc(Nc3ccc4c(C(=O)N(C)C)nn([C@@]5(C(=O)OCc6ccccc6)C[C@@H]5C)c4n3)ncc21]

The crude material (0.5 g, 0.83 mmol) was added to a reaction vial containing HATU (0.65 g, 1.6 mmol) and DIPEA (0.7 mL, 4.1 mmol) in DMF (5 mL). The reaction vial was cooled to 0° C. before dimethylamine (2.0 mL, 4.1 mmol, 2.0 M in THF) was added to this reaction mixture, and it was then sealed and stirred at 23° C. for 2 h. Upon completion, the mixture was diluted with EtOAc and washed with 1N NaOH aq. solution. The organic phase was dried over MgSO₄ and concentrated in vacuo. The crude material was used for the next step without further purification. LCMS calculated for C₃₅H₃₆D₄N₉O₆(M+H)⁺: m/z=686.3; found 686.3.

### Step 6. Methyl ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 37 in step 3 and final purification was done in prep-LCMS (XBridge C18 column, eluting with acetonitrile/aqueous 0.1% NH₄OH, at flow rate of 60 mL/min). LCMS calculated for C₂₈H₃₁D₄N₁₀O₅ (M+H)⁺: m/z=595.3; found 595.4. ¹H NMR (600 MHz, DMSO) δ 10.11 (s, 1H), 8.44 (s, 1H), 8.12-8.02 (m, 2H), 7.36 (s, 1H), 7.26 (s, 1H), 7.16 (d, J=8.8 Hz, 1H), 6.84 (s, 1H), 4.93-4.83 (m, 1H), 3.51 (s, 3H), 3.39 (s, 3H), 3.05 (s, 3H), 2.48-2.40 (m, 1H), 2.24-2.03 (m, 4H), 1.94 (dd, J=13.3, 9.4 Hz, 1H), 1.75 (dd, J=8.3, 4.8 Hz, 1H), 1.69-1.61 (m, 1H), 1.45 (dd, J=9.8, 4.7 Hz, 1H), 1.35 (d, J=6.3 Hz, 3H).

### Example 34. Methyl ((1R,3R)-3-(6-((1-((1S,2S)-1-carbamoyl-2-methylcyclopropyl)-3-morpholino-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

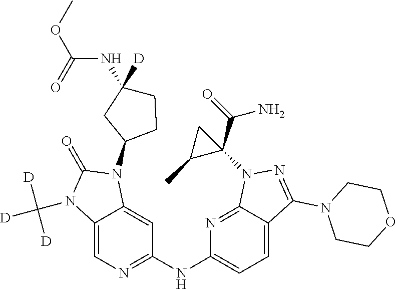 [SMILES: [2H]C([2H])([2H])n1c(=O)n([C@@H]2CC[C@@]([2H])(NC(=O)OC)C2)c2cc(Nc3ccc4c(N5CCOCC5)nn([C@@]5(C(N)=O)C[C@@H]5C)c4n3)ncc21]

### Step 1. Benzyl (1S,2S)-1-(6-chloro-3-iodo-1H-pyrazolo[3,4-b]pyridin-1-yl)-2-methylcyclopropane-1-carboxylate

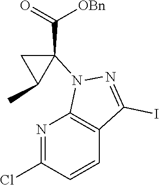 [SMILES: C[C@H]1C[C@]1(C(=O)OCc1ccccc1)n1nc(I)c2ccc(Cl)nc21]

The title compound was prepared according to the procedures described in Example 54, steps 1-6 replacing 6-bromo-2-fluoronicotinonitrile with 6-chloro-2-fluoronicotinonitrile in step 5 followed by the procedure described in step 9 to convert the amino group into iodo. LCMS calculated for C₁₈H₁₆ClIN₃O₂(M+H)⁺: m/z=468.0; found 468.1.

### Step 2. Benzyl (1S,2S)-1-(6-chloro-3-morpholino-1H-pyrazolo[3,4-b]pyridin-1-yl)-2-methylcyclopropane-1-carboxylate

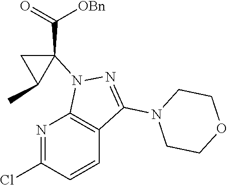 [SMILES: C[C@H]1C[C@]1(C(=O)OCc1ccccc1)n1nc(N2CCOCC2)c2ccc(Cl)nc21]

A vial containing morpholine (0.13 mL, 1.5 mmol), benzyl (1S,2S)-1-(6-chloro-3-iodo-1H-pyrazolo[3,4-b]pyridin-1-yl)-2-methylcyclopropane-1-carboxylate (0.6 g, 1.3 mmol), tris(dibenzylideneacetone)-dipalladium(0) (0.23 g, 0.26 mmol), 4,5-bis(diphenylphosphino)-9,9-dimethylxanthene (0.3 g, 0.51 mmol), and cesium carbonate (1.2 g, 3.8 mmol) was evacuated and backfilled with nitrogen three times, followed by the addition of 1,4-dioxane (10 mL). The vial was sealed and heated to 60° C. for 14 h (or at 100° C. for 1 h). After cooling to r.t., the mixture was filtered and concentrated in vacuo. The crude material was purified by Biotage Isolera to give the desired product. LCMS calculated for C₂₂H₂₄ClN₄O₃(M+H)⁺: m/z=427.1; found 427.1.

### Step 3. Methyl ((1R,3R)-3-(6-((1-((1S,2S)-1-carbamoyl-2-methylcyclopropyl)-3-morpholino-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 54 in step 8 replacing benzyl (1S,2S)-1-(3-(bis(tert-butoxycarbonyl)amino)-6-bromo-1H-pyrazolo[3,4-b]pyridin-1-yl)-2-methylcyclopropane-1-carboxylate with benzyl (1S,2S)-1-(6-chloro-3-morpholino-1H-pyrazolo[3,4-b]pyridin-1-yl)-2-methylcyclopropane-1-carboxylate followed by step 11 and final purification was done in prep-LCMS (XBridge C18 column, eluting with acetonitrile/aqueous 0.1% NH₄OH, at flow rate of 60 mL/min). LCMS calculated for C₂₉H₃₃D₄N₁₀O₅(M+H)⁺: m/z=609.3; found 609.4. ¹H NMR (600 MHz, DMSO) δ 9.89 (s, 1H), 8.40 (s, 1H), 8.09-7.98 (m, 2H), 7.37-7.18 (m, 2H), 6.93 (d, J=8.7 Hz, 1H), 6.40 (s, 1H), 4.92-4.80 (m, 1H), 3.80-3.73 (m, 4H), 3.51 (s, 3H), 3.37-3.32 (m, 4H), 2.47-2.38 (m, 1H), 2.24-2.17 (m, 1H), 2.15-2.04 (m, 2H), 2.01-1.85 (m, 2H), 1.69-1.60 (m, 2H), 1.45 (dd, J=9.5, 4.6 Hz, 1H), 1.29 (d, J=6.4 Hz, 3H).

### Example 35-36

These compounds were prepared according to the procedures described in Example 34, steps 1-3.

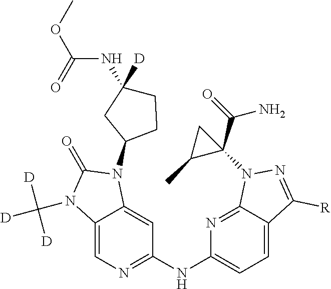

| Example | R | LCMS | NMR |
|---|---|---|---|
| 35 | 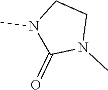 | calculated for C₂₉H₃₂D₄N₁₁O₅ (M + H)⁺: m/z = 622.3; found 622.4 | ¹H NMR (600 MHz, DMSO) δ 9.95 (s, 1H), 8.52-8.37 (m, 2H), 8.05 (s, 1H), 7.30 (s, 1H), 7.24 (s, 1H), 6.94 (d, J = 9.0 Hz, 1H), 6.55 (s, 1H), 4.93-4.80 (m, 1H), 3.97-3.84 (m, 2H), 3.56-3.47 (m, 5H), 2.81 (s, 3H), 2.47-2.39 (m, 1H), 2.24-2.17 (m, 1H), |
| | | | 2.15-2.04 (m, 2H), 2.04-1.96 (m, 1H), |
| | | | 1.92 (dd, J = 13.3, 9.3 Hz, 1H), 1.74-1.61 |
| | | | (m, 2H), 1.46 (dd, J = 9.7, 4.6 Hz, 1H), 1.31 |
| | | | (d, J = 6.2 Hz, 3H) |
| 36 | 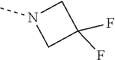 | calculated for C₂₈H₂₉D₄F₂N₁₀O₄ (M + H)⁺: m/z = 615.3; found | ¹H NMR (500 MHz, DMSO) δ 9.94 (s, 1H), 8.39 (s, 1H), 8.05 (s, 1H), 7.83 (d, J = 8.7 Hz, 1H), 7.31 (s, 1H), 7.24 (s, 1H), 6.97 (d, J = 8.8 Hz, 1H), 6.46 (s, 1H), 4.93-4.77 (m, |
| | | 615.5 | 1H), 4.61-4.40 (m, 4H), 3.51 (s, 3H), 2.43 |
| | | | (dd, J = 13.3, 7.5 Hz, 1H), 2.26-2.17 (m, |
| | | | 1H), 2.16-2.04 (m, 2H), 2.03-1.86 (m, |
| | | | 2H), 1.72-1.59 (m, 2H), 1.47 (dd, J = 9.6, |
| | | | 4.5 Hz, 1H), 1.30 (d, J = 6.3 Hz, 3H) |

### Example 37. Methyl ((1R,3R)-3-(6-((1-((1S,2S)-1-carbamoyl-2-methylcyclopropyl)-3-(2-oxopyrrolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

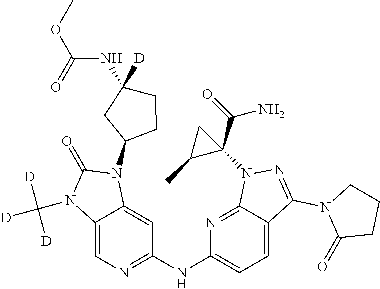 [SMILES: [2H]C([2H])([2H])n1c(=O)n([C@@H]2CC[C@@]([2H])(NC(=O)OC)C2)c2cc(Nc3ccc4c(N5CCCC5=O)nn([C@@]5(C(N)=O)C[C@@H]5C)c4n3)ncc21]

### Step 1. Benzyl (1S,2S)-1-(6-chloro-3-(2-oxopyrrolidin-1-yl)-1H-pyrazolo[3,4-b]pyridin-1-yl)-2-methylcyclopropane-1-carboxylate

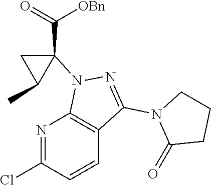 [SMILES: C[C@H]1C[C@]1(C(=O)OCc1ccccc1)n1nc(N2CCCC2=O)c2ccc(Cl)nc21]

A vial containing pyrrolidin-2-one (0.1 mL, 1.3 mmol), benzyl (1S,2S)-1-(6-chloro-3-iodo-1H-pyrazolo[3,4-b]pyridin-1-yl)-2-methylcyclopropane-1-carboxylate (0.6 g, 1.3 mmol) (this compound was prepared according to the procedures described in Example 34, steps 1), tris(dibenzylideneacetone)-dipalladium(0) (0.23 g, 0.26 mmol), 4,5-bis(diphenylphosphino)-9,9-dimethylxanthene (0.3 g, 0.51 mmol), and cesium carbonate (1.2 g, 3.8 mmol) was evacuated and backfilled with nitrogen three times, followed by the addition of 1,4-dioxane (10 mL). The vial was sealed and heated to 60° C. for 14 h. After cooling to r.t., the mixture was filtered and concentrated in vacuo. The crude material was purified by Biotage Isolera to give the desired product. LCMS calculated for C₂₂H₂₂ClN₄O₃(M+H)⁺: m/z=425.1; found 425.2.

### Step 2. Benzyl (1S,2S)-1-(6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-3-(2-oxopyrrolidin-1-yl)-1H-pyrazolo[3,4-b]pyridin-1-yl)-2-methylcyclopropane-1-carboxylate

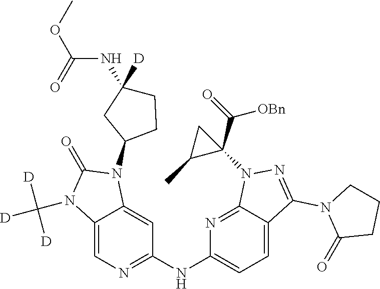 [SMILES: [2H]C([2H])([2H])n1c(=O)n([C@@H]2CC[C@@]([2H])(NC(=O)OC)C2)c2cc(Nc3ccc4c(N5CCCC5=O)nn([C@@]5(C(=O)OCc6ccccc6)C[C@@H]5C)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (0.32 g, 1.0 mmol), benzyl (1S,2S)-1-(6-chloro-3-(2-oxopyrrolidin-1-yl)-1H-pyrazolo[3,4-b]pyridin-1-yl)-2-methylcyclopropane-1-carboxylate (0.42 g, 1.0 mmol), tris(dibenzylideneacetone)-dipalladium(0) (0.18 g, 0.2 mmol), 4,5-bis(diphenylphosphino)-9,9-dimethylxanthene (0.23 g, 0.4 mmol), and cesium carbonate (1.0 g, 3.0 mmol) was evacuated and backfilled with nitrogen three times, followed by the addition of 1,4-dioxane (10 mL). The vial was sealed and heated to 100° C. for 4 h. After cooling to r.t., the mixture was filtered and concentrated in vacuo. The crude material was purified by Biotage Isolera to give the desired product. LCMS calculated for C₃₆H₃₆D₄N₉O₆(M+H)⁺: m/z=698.3; found 698.4.

### Step 3. Methyl ((1R,3R)-3-(6-((1-((1S,2S)-1-carbamoyl-2-methylcyclopropyl)-3-(2-oxopyrrolidin-1-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

A mixture of benzyl (1S,2S)-1-(6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-3-(2-oxopyrrolidin-1-yl)-1H-pyrazolo[3,4-b]pyridin-1-yl)-2-methylcyclopropane-1-carboxylate (0.63 g, 0.9 mmol) and Pd/C (100 mg, 10% Wt) in MeOH (9 mL) under H₂ balloon was stirred at 60° C. for 3 hours. The mixture was filtered through celite and concentrated in vacuo. The crude material was added to a reaction vial containing HATU (0.68 g, 1.8 mmol) and DIPEA (0.8 mL, 4.5 mmol) in DMF (9 mL). The reaction vial was cooled to 0° C. before ammonia (11 mL, 4.5 mmol, 0.4 M in dioxane) was added to this reaction mixture, and it was then stirred at room temperature for 2 h. Upon completion, the mixture was diluted with EtOAc and washed with 1N NaOH aq. solution. The organic phase was dried over MgSO₄ and concentrated in vacuo. The final purification was done in prep-LCMS (XBridge C18 column, eluting with acetonitrile/aqueous 0.10% NH₄OH, at flow rate of 60 mL/min). LCMS calculated for C₂₉H₃₁D₄N₁₀O₅ (M+H)⁺: m/z=607.3; found 607.4. ¹H NMR (600 MHz, DMSO) δ 10.00 (s, 1H), 8.49-8.34 (m, 2H), 8.06 (s, 1H), 7.32 (s, 1H), 7.24 (s, 1H), 6.99 (d, J=9.0 Hz, 1H), 6.61 (s, 1H), 4.94-4.80 (m, 1H), 4.03-3.90 (m, 2H), 3.51 (s, 3H), 2.57-2.51 (m, 2H), 2.44 (dd, J=13.4, 7.4 Hz, 1H), 2.25-1.98 (m, 6H), 1.92 (dd, J=13.3, 9.4 Hz, 1H), 1.75-1.61 (m, 2H), 1.46 (dd, J=9.7, 4.6 Hz, 1H), 1.32 (d, J=6.3 Hz, 3H).

### Example 38. Methyl ((1R,3R)-3-(6-((1-((1S,2R)-1-carbamoyl-2-ethynylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

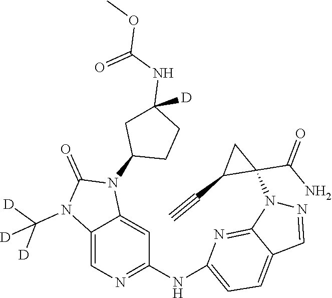 [SMILES: [2H]C([2H])([2H])n1c(=O)n([C@@H]2CC[C@@]([2H])(NC(=O)OC)C2)c2cc(Nc3ccc4cnn([C@@]5(C(N)=O)C[C@@H]5C#C)c4n3)ncc21]

### Step 1. Methyl (1S,2R)-1-((tert-butoxycarbonyl)amino)-2-vinylcyclopropane-1-carboxylate

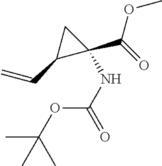 [SMILES: C=C[C@H]1C[C@@]1(NC(=O)OC(C)(C)C)C(=O)OC]

To a solution of (1S,2R)-1-((tert-butoxycarbonyl)amino)-2-vinylcyclopropane-1-carboxylic acid (7.5 g, 33 mmol [Ambeed, A2668944]) in methanol (25 ml) and toluene (100 ml) was added TMS-diazomethane (18 ml, 36 mmol, 2M in hexanes, dropwise). After 1 hour, acetic acid (0.2 ml, 3.3 mmol) was added and volatiles were removed in vacuo to furnish the desired methyl ester as yellow oil (7.9 g, 33 mmol, 99% yield). LCMS calculated for C₈H₁₂NO₄ (M-(tBu)+H)⁺: m/z=186.1; found 186.1.

### Step 2. Methyl (1S,2R)-1-(bis(tert-butoxycarbonyl)amino)-2-vinylcyclopropane-1-carboxylate

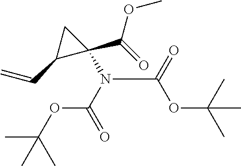 [SMILES: C=C[C@H]1C[C@]1(C(=O)OC)N(C(=O)OC(C)(C)C)C(=O)OC(C)(C)C]

To a solution of methyl (1S,2R)-1-((tert-butoxycarbonyl)amino)-2-vinylcyclopropane-1-carboxylate (2 g, 8.3 mmol) in THF (38 ml) was added lithium bis(trimethylsilyl)amide (9.1 ml, 9.1 mmol). The mixture was stirred at ambient temperature for 10 minutes then boc-anhydride (2.3 ml, 10.0 mmol) was added. After 1 hour, the mixture was diluted with EtOAc (100 mL) and water (50 mL). The phases were separated, and the organic phase was washed with brine (50 mL), dried over sodium sulfate and concentrated in vacuo. The resulting crude residue was purified by Biotage Isolera to give the desired product as a colorless oil (2.8 g, 98%). LCMS calculated for C₃₄H₅₄N₂O₁₂Na (2M+Na)⁺: m/z=705.4; found 705.3.

### Step 3: Methyl (1S,2S)-1-(bis(tert-butoxycarbonyl)amino)-2-formylcyclopropane-1-carboxylate

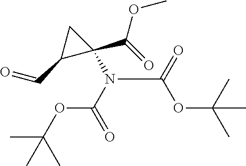 [SMILES: COC(=O)[C@]1(N(C(=O)OC(C)(C)C)C(=O)OC(C)(C)C)C[C@@H]1C=O]

To a solution of methyl (1S,2R)-1-(bis(tert-butoxycarbonyl)amino)-2-vinylcyclopropane-1-carboxylate (2.3 g, 6.7 mmol) in i-PrOH (25 ml) and water (25 ml) was added sodium periodate (4.3 g, 20.2 mmol) followed by potassium osmate dihydrate (0.1 g, 0.3 mmol). After stirring for 3 hours, the mixture was diluted with EtOAc (200 mL) and a saturated solution of NaHCO₃ (50 mL). The phases were separated, and the organic phase was washed with a saturated solution of sodium thiosulfate. Combined organic phases were washed with brine (50 mL), dried over sodium sulfate, and concentrated in vacuo. The resulting crude residue was purified by Biotage Isolera to give the desired product as a colorless oil (1.6 g, 69%). LCMS calculated for C₃₂H₅₀N₂O₁₄Na (2M+Na)⁺: m/z=709.3; found 709.3. ¹H NMR (400 MHz, CDCl₃) δ 9.45 (d, J=5.7, 1H), 3.77 (s 3H), 2.47 (dd, J=8.5, 6.4 Hz, 1H), 2.39-2.25 (m, 1H), 1.78 (dd, J=9.6, 6.4 Hz, 1H), 1.48 (s, 18H).

### Step 4: Methyl (1S,2R)-1-(bis(tert-butoxycarbonyl)amino)-2-ethynylcyclopropane-1-carboxylate

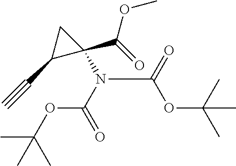 [SMILES: C#C[C@H]1C[C@]1(C(=O)OC)N(C(=O)OC(C)(C)C)C(=O)OC(C)(C)C]

To a solution of methyl (1S,2S)-1-(bis(tert-butoxycarbonyl)amino)-2-formylcyclopropane-1-carboxylate (1.7 g, 5.0 mmol) in MeOH (40 mL) was added potassium carbonate (1.5 g, 10.9 mmol). The resulting suspension was cooled to 4° C., then dimethyl (1-diazo-2-oxopropyl)phosphonate (0.966 mL, 6.44 mmol) was added dropwise. The mixture was warmed to ambient temperature, stirred for 20 hours, filtered, and concentrated in vacuo. The crude material was purified by Biotage Isolera to give the desired product as a colorless oil (910 mg, 54%). ¹H NMR (400 MHz, CDCl₃) δ 3.76 (s, 3H), 2.27-2.18 (m, 1H), 2.13-2.06 (m, 2H), 1.50 (s, 18H), 1.44 (dd, J=9.9, 5.7 Hz, 1H).

### Step 5: Methyl (1S,2R)-1-amino-2-ethynylcyclopropane-1-carboxylate hydrochloride

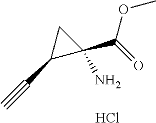

To a solution of methyl (1S,2R)-1-(bis(tert-butoxycarbonyl)amino)-2-ethynylcyclopropane-1-carboxylate (910 mg, 2.7 mmol) in DCM (3 mL) was added HCl (4M in dioxane) (19 mL, 77 mmol) and the resulting mixture was stirred at ambient temperature. After 4 hours the volatiles were removed in vacuo to furnish the desired product as an off-white crystalline solid (471 mg, 100%). LCMS calculated for C₇H₁₀NO₂ (M+H)⁺: m/z=140.1; found 140.1. ¹H NMR (400 MHz, DMSO) δ 9.15 (s, 3H), 3.77 (s, 3H), 3.09 (d, J=2.2 Hz, 1H), 2.56-2.50 (m, 1H—overlapping with solvent signal), 1.87 (dd, J=10.1, 5.9 Hz, 1H), 1.77 (dd, J=7.8, 5.9 Hz, 1H).

### Step 6: Methyl ((1R,3R)-3-(6-((1-((1S,2R)-1-carbamoyl-2-ethynylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 16 using methyl (1S,2R)-1-amino-2-ethynylcyclopropane-1-carboxylate hydrochloride in the place of rac-methyl (1R,2R)-1-amino-2-methylcyclopropane-1-carboxylate hydrochloride in Step 2, and final purification was done in prep-LCMS (XBridge C18 column, eluting with acetonitrile/aqueous 0.1% NH₄OH, at flow rate of 60 mL/min). LCMS calculated for C₂₆H₂₄D₄N₉O₄(M+H)⁺: m/z=534.2; found 534.3. ¹H NMR (600 MHz, DMSO) δ 9.97 (s, 1H), 8.23 (s, 1H), 8.05 (s, 1H), 7.97 (s, 1H), 7.96 (s, 1H), 7.38 (s, 1H), 7.22 (d, J=8.7 Hz, 1H), 7.17 (s, 1H), 6.59 (s, 1H), 4.86 (t, J=8.5 Hz, 1H), 3.52 (s, 3H), 2.93 (d, J=2.3 Hz, 1H), 2.63 (ddd, J=10.1, 7.9, 2.3 Hz, 1H), 2.41 (dd, J=13.4, 7.4 Hz, 1H), 2.24-2.15 (m, 2H), 2.14-2.05 (m, 2H), 1.91 (dd, J=13.4, 9.4 Hz, 1H), 1.80 (dd, J=9.9, 4.8 Hz, 1H), 1.63 (ddd, J=12.5, 9.3, 7.8 Hz, 1H).

### Example 39. Methyl ((1R,3R)-3-(6-((1-((1S,2S)-1-carbamoyl-2-(trifluoromethyl)cyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

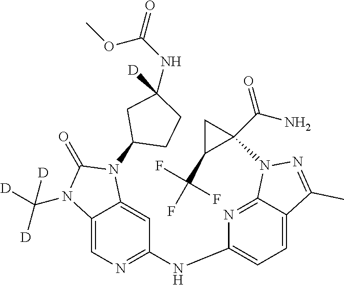 [SMILES: [2H]C([2H])([2H])n1c(=O)n([C@@H]2CC[C@@]([2H])(NC(=O)OC)C2)c2cc(Nc3ccc4c(C)nn([C@@]5(C(N)=O)C[C@@H]5C(F)(F)F)c4n3)ncc21]

### Step 1. Diethyl 2-(trifluoromethyl)cyclopropane-1,1-dicarboxylate

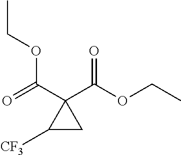 [SMILES: CCOC(=O)C1(C(=O)OCC)CC1C(F)(F)F]

To a stirred suspension of sodium hydride (0.5 g, 12.5 mmol) in dry DME (25 mL) was added dropwise diethyl malonate (0.76 ml, 5 mmol) at 24° C. over 5 min under an N₂ atmosphere. A solution of 4-(trifluoromethyl)-1,3,2-dioxathiolane 2,2-dioxide (1.1 g, 5.5 mmol) in dry DME (5 mL) was slowly added to the malonate solution, and the resulting mixture was refluxed overnight. After cooling to r.t, brine (10 mL) was added slowly, the mixture was extracted with Et₂O. The organic layers were combined, dried over Na₂SO₄, filtered, and the solvents were evaporated in vacuo. The crude material was purified by Biotage Isolera. ¹H NMR (400 MHz, CDCl₃) δ 4.50-3.98 (m, 4H), 2.71-2.46 (m, 1H), 1.82 (dd, J=7.3, 5.6 Hz, 1H), 1.62-1.54 (m, 1H), 1.31 (t, J=7.2 Hz, 6H).

### Step 2. 1-(Ethoxycarbonyl)-2-(trifluoromethyl)cyclopropane-1-carboxylic acid

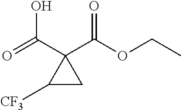

To a flask containing a solution of diethyl 2-(trifluoromethyl)cyclopropane-1,1-dicarboxylate (1.1 g, 4.3 mmol) in EtOH (12 mL) and water (3 mL) was added dropwise NaOH (5 mL, 5.0 mmol, 1.0 M in water) at r.t. The mixture was stirred for 5 h and EtOH was evaporated in vacuo. The resulting aqueous layer was acidified with 1M HCl and extracted with EtOAc. The combined organic layers were washed with brine, dried over Na₂SO₄, filtered, and the solvents were evaporated in vacuo. The crude material was used directly in the next step without further purification. ¹H NMR (400 MHz, CDCl₃) δ 4.31 (q, J=7.1 Hz, 2H), 2.83-2.57 (m, 1H), 2.07 (dd, J=7.6, 5.6 Hz, 1H), 1.86 (dd, J=9.8, 5.5 Hz, 1H), 1.32 (t, J=7.2 Hz, 3H).

### Step 3. Ethyl (1S,2S)-1-(((benzyloxy)carbonyl)amino)-2-(trifluoromethyl)cyclopropane-1-carboxylate

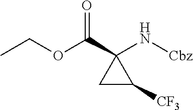 [SMILES: CCOC(=O)[C@]1(NC(=O)OCc2ccccc2)C[C@@H]1C(F)(F)F]

To a flask containing 1-(ethoxycarbonyl)-2-(trifluoromethyl)cyclopropane-1-carboxylic acid (0.68 g, 3 mmol) in toluene (20 mL) was added triethylamine (0.75 mL, 5.4 mmol) and diphenyl phosphorazidate (0.71 mL, 3.3 mmol). After heating at 50° C. for 20 min, the reaction mixture was cooled down to r.t., followed by the addition of phenylmethanol (0.47 mL, 4.5 mmol) and DMAP (5 mg). The reaction mixture was then heated to 95° C. for 16 h. The reaction mixture was cooled down to r.t, concentrated in vacuo and purified by Teledyne ISCO CombiFlash to give a mixture of two isomers. The mixture was further separated by chiral SFC ((R, R) Regis Whelk-01, 21.1 mm×250 mm, 5 um column, eluting with 4% methanol in CO₂, at flow rate of 65 mL/min over 7.5 min). The desired isomer was collected at 5.6 min as a single compound. LCMS calculated for C₁₅H₁₇F₃NO₄ (M+H)⁺: m/z=332.1; found: 332.1.

### Step 4. Ethyl (1S,2S)-1-amino-2-(trifluoromethyl)cyclopropane-1-carboxylate

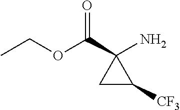

To a vial containing ethyl (1S,2S)-1-(((benzyloxy)carbonyl)amino)-2-(trifluoromethyl)cyclopropane-1-carboxylate (0.28 g, 0.85 mmol) in MeOH (5 mL) was added Pd/C (20 mg, 10% w/w). The vial was purged with hydrogen gas for 5 min and then stirred for 5 h under an atmosphere of hydrogen at r.t. After cooling to room temperature, the reaction mixture was filtered through Celite and washed with CH₂Cl₂, followed by concentration of the filtrate in vacuo. The crude product was used directly in the next step without further purification. LCMS calculated for C₇H₁₁F₃NO₂ (M+H)⁺: m/z=198.1; found: 198.1

### Step 5. Methyl ((1R,3R)-3-(6-((1-((1S,2S)-1-carbamoyl-2-(trifluoromethyl)cyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 54, Step 5-11, with ethyl (1S,2S)-1-amino-2-(trifluoromethyl)cyclopropane-1-carboxylate replacing benzyl (1S,2S)-1-amino-2-methylcyclopropane-1-carboxylate in Step 5. LCMS calculated for: C₂₆H₂₅D₄F₃N₉O₄ (M+H)⁺: m/z=592.3; found 592.3. ¹H NMR (500 MHz, DMSO) δ 11.31 (s, 1H), 8.22 (d, J=8.6 Hz, 1H), 8.10 (s, 1H), 7.66 (s, 1H), 7.57 (s, 2H), 7.29 (s, 1H), 7.02 (d, J=8.6 Hz, 1H), 5.11-4.80 (m, 1H), 3.55 (s, 3H), 3.22-3.00 (m, 1H), 2.49 (s, 3H), 2.39-2.28 (m, 2H), 2.24-2.02 (m, 3H), 2.01-1.91 (m, 2H), 1.72-1.58 (m, 1H).

### Example 40. Methyl ((1R,3R)-3-(6-((1-((1S,2S)-1-carbamoyl-2-(trifluoromethyl)cyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

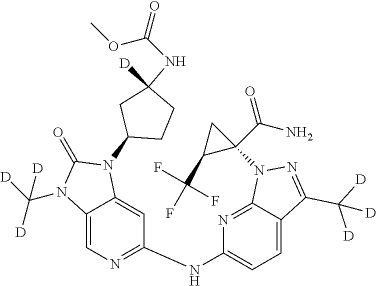 [SMILES: [2H]C([2H])([2H])c1nn([C@@]2(C(N)=O)C[C@@H]2C(F)(F)F)c2nc(Nc3cc4c(cn3)n(C([2H])([2H])[2H])c(=O)n4[C@@H]3CC[C@@]([2H])(NC(=O)OC)C3)ccc12]

The title compound was prepared according to the procedures described in Example 39, with methyl-d₃-zinc chloride replacing methylzinc chloride. LCMS calculated for: C₂₆H₂₂D₇F₃N₉O₄ (M+H)⁺: m/z=595.3; found 595.3. ¹H NMR (500 MHz, DMSO) δ 11.41 (s, 1H), 8.22 (d, J=8.6 Hz, 1H), 8.11 (s, 1H), 7.67 (s, 1H), 7.57 (s, 2H), 7.29 (s, 1H), 7.03 (d, J=8.6 Hz, 1H), 5.31-4.81 (m, 1H), 3.55 (s, 3H), 3.12-3.04 (m, 1H), 2.38-2.29 (m, 2H), 2.23-2.02 (m, 3H), 2.01-1.90 (m, 2H), 1.71-1.51 (m, 1H).

### Example 41. Methyl ((1R,3R)-3-(6-((1-((1S,2S)-1-carbamoyl-2-(trifluoromethyl)cyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

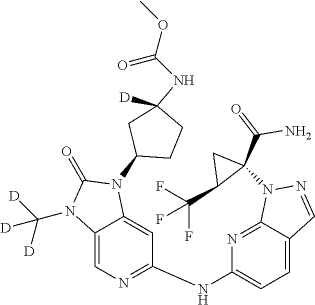 [SMILES: [2H]C([2H])([2H])n1c(=O)n([C@@H]2CC[C@@]([2H])(NC(=O)OC)C2)c2cc(Nc3ccc4cnn([C@@]5(C(N)=O)C[C@@H]5C(F)(F)F)c4n3)ncc21]

The title compound was prepared according to the procedures described in Example 16 using rac-methyl (1R,2R)-1-amino-2-(trifluoromethyl)cyclopropane-1-carboxylate hydrochloride in place of rac-methyl (1R,2R)-1-amino-2-methylcyclopropane-1-carboxylate hydrochloride. Final purification was done by prep-LCMS (XBridge C18 column, eluting with acetonitrile/aqueous 0.1% NH₄OH, at flow rate of 60 mL/min). The active diastereomer eluted second at a retention time of 5.5 min. LCMS calculated for C₂₅H₂₃D₄F₃N₉O₄ (M+H)⁺: m/z=578.2; found 578.3. ¹H NMR (600 MHz, DMSO) δ 10.02 (s, 1H), 8.28 (s, 1H), 8.07 (s, 1H), 8.02-7.96 (m, 2H), 7.56 (s, 1H), 7.17 (d, J=8.8 Hz, 1H), 7.09 (s, 1H), 6.84 (s, 1H), 5.03-4.95 (m, 1H), 3.52 (s, 3H), 3.04-2.98 (m, 1H), 2.45-2.36 (m, 2H), 2.22-2.16 (m, 1H), 2.15-2.05 (m, 2H), 2.01-1.96 (m, 1H), 1.90 (dd, J=13.4, 9.6 Hz, 1H), 1.61-1.54 (m, 1H).

### Example 42. Methyl ((1R,3R)-3-(6-((1-((1S,2S)-1-carbamoyl-2-(difluoromethyl)cyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

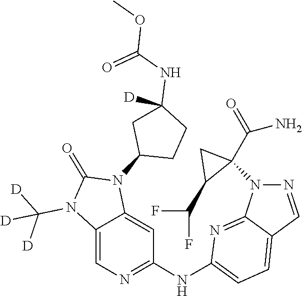 [SMILES: [2H]C([2H])([2H])n1c(=O)n([C@@H]2CC[C@@]([2H])(NC(=O)OC)C2)c2cc(Nc3ccc4cnn([C@@]5(C(N)=O)C[C@@H]5C(F)F)c4n3)ncc21]

### Step 1. Methyl (1S,2R)-1-((tert-butoxycarbonyl)amino)-2-vinylcyclopropane-1-carboxylate

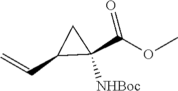

To a solution of (1S,2R)-1-((tert-butoxycarbonyl)amino)-2-vinylcyclopropane-1-carboxylic acid (9.0 g, 39.6 mmol), DMAP (0.97 g, 7.9 mmol) and MeOH (16 mL, 396 mmol) in MeCN (90 ml) at 0° C. was added EDC (9.11 g, 47.5 mmol). The mixture was warmed to room temperature and stirred for 3 hours. The mixture was concentrated, diluted with aq. sodium bicarbonate and extracted 2 times with EtOAc. The combined organic phases were dried over sodium sulfate, and filtered through a silica plug with EtOAc. The filtrate was concentrated to give the desired product as an oil. ¹H NMR (400 MHz, CDCl₃) δ 5.81-5.70 (m, 1H), 5.29 (d, J=17.1 Hz, 1H), 5.13 (d, J=10.3 Hz, 1H), 3.73 (s, 3H), 2.16 (q, J=8.8 Hz, 1H), 1.85-1.75 (m, 1H), 1.55-1.42 (m, 10H)

### Steps 2-5. Methyl (1S,2S)-1-amino-2-(difluoromethyl)cyclopropane-1-carboxylate hydrochloride

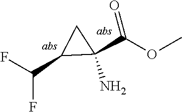

The above compound was prepared starting from methyl (1S,2R)-1-((tert-butoxycarbonyl)amino)-2-vinylcyclopropane-1-carboxylate following the procedures outlined in WO2009064975 (Scheme 8).

### Steps 6-11. Methyl ((1R,3R)-3-(6-((1-((1S,2S)-1-carbamoyl-2-(difluoromethyl)cyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

The title compound was prepared according to the procedures described in Example 16 using methyl (1S,2S)-1-amino-2-(difluoromethyl)cyclopropane-1-carboxylate hydrochloride in place of rac-methyl (1R,2R)-1-amino-2-methylcyclopropane-1-carboxylate hydrochloride. Final purification was done by prep-LCMS (Waters Phenyl Hexyl column, eluting with a gradient of acetonitrile/water containing 0.1% TFA, at flow rate of 60 mL/min). LCMS calculated for C₂₅H₂₄D₄F₂N₉O₄ (M+H)⁺: m/z=560.2; found 560.3. ¹H NMR (600 MHz, DMSO) δ 8.17-8.13 (m, 2H), 8.09 (s, 1H), 7.89 (s, 1H), 7.67 (s, 1H), 7.28 (s, 1H), 7.09 (d, J=8.7 Hz, 1H), 7.00 (s, 1H), 6.38-6.14 (m, 1H), 4.90-4.84 (m, 1H), 3.54 (s, 3H), 2.60-2.52 (m, 1H), 2.37 (dd, J=13.6, 7.4 Hz, 1H), 2.26-2.12 (m, 3H), 2.09-2.00 (m, 1H), 2.00-1.93 (m, 2H), 1.69-1.62 (m, 1H)

### Example 43. Methyl ((1R,3R)-3-(6-((1-((1S,2S)-1-carbamoyl-2-ethylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

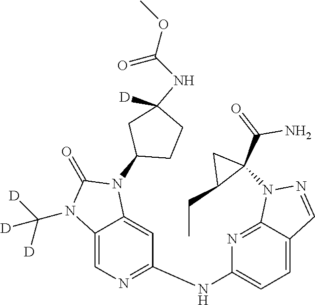 [SMILES: [2H]C([2H])([2H])n1c(=O)n([C@@H]2CC[C@@]([2H])(NC(=O)OC)C2)c2cc(Nc3ccc4cnn([C@@]5(C(N)=O)C[C@@H]5CC)c4n3)ncc21]

### Step 1. Methyl (1S,2S)-1-((tert-butoxycarbonyl)amino)-2-ethylcyclopropane-1-carboxylate

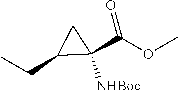

A mixture of methyl (1S,2R)-1-((tert-butoxycarbonyl)amino)-2-vinylcyclopropane-1-carboxylate (10.6 g, 43.9 mmol) and Pd/C (530 mg, 10% Wt) in MTBE (200 mL) under an H₂ balloon was stirred at room temperature for 2.5 hours. The mixture was filtered through celite, concentrated, and purified by chromatography (Biotage Isolera, pure Hex to 15% EtOAc) to give the desired product as an oil. ¹H NMR (400 MHz, CDCl₃) δ 3.73 (s, 3H), 1.63-1.53 (m, 2H), 1.51-1.42 (m, 12H), 0.97 (t, J=7.3 Hz, 3H).

### Step 2. Methyl (1S,2S)-1-amino-2-ethylcyclopropane-1-carboxylate hydrochloride

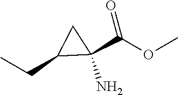

A solution of methyl (1S,2S)-1-((tert-butoxycarbonyl)amino)-2-ethylcyclopropane-1-carboxylate (5.1 g, 21.0 mmol) in DCM (25 mL) and HCl (52 mL, 4 M in dioxane) was stirred at room temperature for 2 hours. The mixture was then concentrated to give a solid which was used directly in the next step.

### Steps 3-8. Methyl ((1R,3R)-3-(6-((1-((1S,2S)-1-carbamoyl-2-ethylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

The title compound was prepared according to the procedures described in Example 16 using methyl (1S,2S)-1-amino-2-ethylcyclopropane-1-carboxylate hydrochloride in place of rac-methyl (1R,2R)-1-amino-2-methylcyclopropane-1-carboxylate hydrochloride. Final purification was done by prep-LCMS (XBridge C18 column, eluting with acetonitrile/aqueous 0.1% NH₄OH, at flow rate of 60 mL/min). LCMS calculated for C₂₆H₂₈D₄N₉O₄(M+H)⁺: m/z=538.3; found 538.3. ¹H NMR (600 MHz, DMSO) δ 10.01 (s, 1H), 8.50 (s, 1H), 8.06 (s, 1H), 7.96-7.93 (m, 2H), 7.35 (s, 1H), 7.26 (s, 1H), 7.09 (d, J=8.7 Hz, 1H), 6.38 (s, 1H), 4.95-4.88 (m, 1H), 3.52 (s, 3H), 2.46 (dd, J=13.3, 7.6 Hz, 1H), 2.25-2.19 (m, 1H), 2.17-2.02 (m, 3H), 1.95 (dd, J=13.3, 9.3 Hz, 1H), 1.79 (dd, J=8.3, 4.7 Hz, 1H), 1.74-1.62 (m, 3H), 1.44 (dd, J=9.6, 4.7 Hz, 1H), 1.05 (t, J=7.4 Hz, 3H).

### Example 44. Methyl ((1R,3R)-3-(6-((1-((1S,2S)-1-carbamoyl-2-ethylcyclopropyl)-3-(3,3-difluoroazetidin-1-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

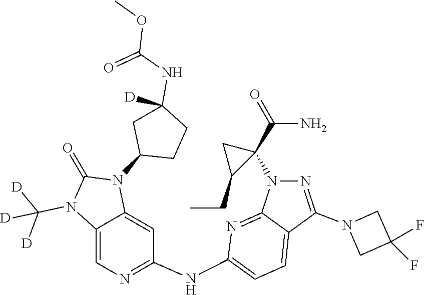 [SMILES: [2H]C([2H])([2H])n1c(=O)n([C@@H]2CC[C@@]([2H])(NC(=O)OC)C2)c2cc(Nc3ccc4c(N5CC(F)(F)C5)nn([C@@]5(C(N)=O)C[C@@H]5CC)c4n3)ncc21]

The title compound was prepared according to the procedure described in Examples 34 and 36 using methyl (1S,2S)-1-amino-2-ethylcyclopropane-1-carboxylate hydrochloride in place of benzyl (1S,2S)-1-amino-2-methylcyclopropane-1-carboxylate. Final purification was done by prep-LCMS (Waters Phenyl Hexyl column, eluting with a gradient of acetonitrile/water containing 0.1% TFA, at flow rate of 60 mL/min). LCMS calculated for C₂₉H₃₁D₄F₂N₁₀O₄ (M+H)⁺: m/z=629.3; found 629.3. ¹H NMR (500 MHz, DMSO) δ 10.96 (s, 1H), 8.23 (s, 1H), 8.03 (d, J=8.6 Hz, 1H), 7.83 (s, 1H), 7.35 (s, 1H), 7.31 (s, 1H), 6.89 (d, J=8.7 Hz, 1H), 6.66 (s, 1H), 5.00-4.90 (m, 1H), 4.64-4.46 (m, 4H), 3.53 (s, 3H), 2.38 (dd, J=13.5, 7.6 Hz, 1H), 2.26-2.01 (m, 3H), 2.01-1.91 (m, 2H), 1.82 (dd, J=8.1, 4.7 Hz, 1H), 1.76-1.54 (m, 3H), 1.47 (dd, J=9.7, 4.7 Hz, 1H), 1.04 (t, J=7.4 Hz, 3H).

### Example 45. Methyl ((1R,3R)-3-(6-((1-((1R,2S)-2-carbamoyl-[1,1′-bi(cyclopropan)]-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

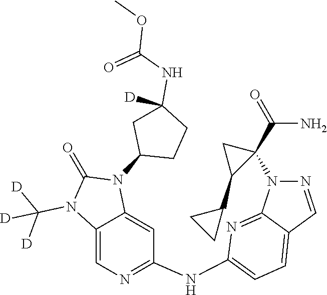 [SMILES: [2H]C([2H])([2H])n1c(=O)n([C@@H]2CC[C@@]([2H])(NC(=O)OC)C2)c2cc(Nc3ccc4cnn([C@@]5(C(N)=O)C[C@@H]5C5CC5)c4n3)ncc21]

### Step 1. Methyl (1R,2S)-2-amino-[1,1′-bi(cyclopropane)]-2-carboxylate

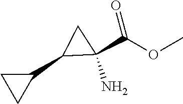

To a solution of diethylzinc (4.6 mL, 4.6 mmol, 1M in hexanes) in DCM (26 mL) at 0° C. was added TFA (0.28 mL, 3.6 mmol) in DCM (5 mL) dropwise over 1 hour. The mixture was stirred for another 20 minutes and then CH₂I₂(0.37 mL, 4.6 mmol) in DCM (2 mL) was added over 15 minutes. The mixture was stirred for another 20 minutes, and then methyl (1S,2R)-1-((tert-butoxycarbonyl)amino)-2-vinylcyclopropane-1-carboxylate (267 mg, 1.10 mmol) in DCM (5 mL) was added over 5 minutes. The mixture was warmed to RT and stirred overnight. The mixture was quenched with aq NH₄Cl and then extracted with DCM. The organic phase was dried over sodium sulfate, and then filtered/concentrated to give the de-boc product as an oil which was used directly in the next step. ¹H NMR (400 MHz, CDCl₃) 3.76 (s, 3H), 1.43 (dd, J=7.6, 4.8 Hz, 1H), 1.25-1.19 (m, 1H), 1.09-1.02 (m, 1H), 0.83-0.72 (m, 1H), 0.57-0.42 (m, 2H), 0.34-0.17 (m, 2H).

### Steps 2-7. Methyl ((1R,3R)-3-(6-((1-((1R,2S)-2-carbamoyl-[1,1′-bi(cyclopropan)]-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 16 using methyl (1R,2S)-2-amino-[1,1′-bi(cyclopropane)]-2-carboxylate in place of rac-methyl (1R,2R)-1-amino-2-methylcyclopropane-1-carboxylate hydrochloride. Final purification was done by prep-LCMS (Waters Sunfire column, eluting with a gradient of acetonitrile/water containing 0.1% TFA, at flow rate of 60 mL/min). LCMS calculated for C₂₇H₂₈D₄N₉O₄(M+H)⁺: m/z=550.3; found 550.3. ¹H NMR (500 MHz, DMSO) δ 11.02 (s, 1H), 8.21 (s, 1H), 8.16 (d, J=8.6 Hz, 1H), 8.05 (s, 1H), 7.87 (s, 1H), 7.41 (s, 1H), 7.33 (s, 1H), 7.02 (d, J=8.7 Hz, 1H), 6.83 (s, 1H), 5.01-4.91 (m, 1H), 3.52 (s, 3H), 2.38 (dd, J=13.6, 7.5 Hz, 1H), 2.26-2.12 (m, 2H), 2.10-2.02 (m, 1H), 2.00-1.92 (m, 2H), 1.71-1.62 (m, 2H), 1.47 (dd, J=9.9, 5.1 Hz, 1H), 1.10-1.00 (m, 1H), 0.60-0.49 (m, 2H), 0.41-0.31 (m, 2H).

### Example 46. Methyl ((1R,3R)-3-(6-((1-((1S,2S)-1-carbamoyl-2-ethylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

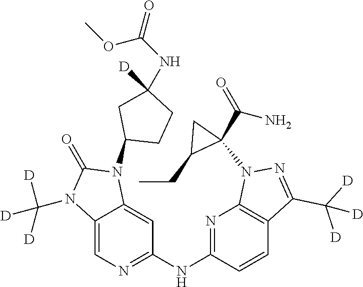 [SMILES: [2H]C([2H])([2H])c1nn([C@@]2(C(N)=O)C[C@@H]2CC)c2nc(Nc3cc4c(cn3)n(C([2H])([2H])[2H])c(=O)n4[C@@H]3CC[C@@]([2H])(NC(=O)OC)C3)ccc12]

The title compound was prepared according to the procedures described in Examples 54 and 55 with methyl (1S,2S)-1-amino-2-ethylcyclopropane-1-carboxylate hydrochloride replacing benzyl (1S,2S)-1-amino-2-methylcyclopropane-1-carboxylate. Final purification was done by prep-LCMS (XBridge C18 column, eluting with acetonitrile/aqueous 0.1% NH₄OH, at flow rate of 60 mL/min). LCMS calculated for C₂₇H₂₇D₇N₉O₄(M+H)⁺: m/z=555.3; found 555.3. ¹H NMR (600 MHz, DMSO) δ 9.96 (s, 1H), 8.49 (s, 1H), 8.06 (s, 1H), 7.92 (d, J=8.7 Hz, 1H), 7.32 (s, 1H), 7.25 (s, 1H), 7.04 (d, J=8.7 Hz, 1H), 6.37 (s, 1H), 4.94-4.88 (m, 1H), 3.52 (s, 3H), 2.46 (dd, J=13.2, 7.7 Hz, 1H), 2.27-2.19 (m, 1H), 2.15-2.07 (m, 2H), 2.05-1.99 (m, 1H), 1.97-1.90 (m, 1H), 1.76 (dd, J=8.2, 4.7 Hz, 1H), 1.73-1.61 (m, 3H), 1.41 (dd, J=9.6, 4.6 Hz, 1H), 1.04 (t, J=7.4 Hz, 3H).

### Example 47. Methyl ((1R,3R)-3-(6-((1-((1R,2S)-2-carbamoyl-[1,1′-bi(cyclopropan)]-2-yl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

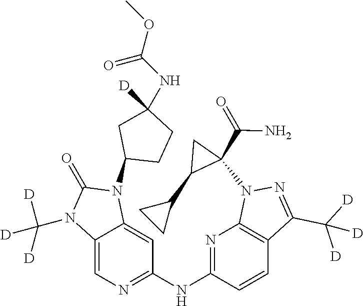 [SMILES: [2H]C([2H])([2H])c1nn([C@@]2(C(N)=O)C[C@@H]2C2CC2)c2nc(Nc3cc4c(cn3)n(C([2H])([2H])[2H])c(=O)n4[C@@H]3CC[C@@]([2H])(NC(=O)OC)C3)ccc12]

The title compound was prepared according to the procedures described in Examples 54 and 55 with methyl (1R,2S)-2-amino-[1,1′-bi(cyclopropane)]-2-carboxylate replacing benzyl (1S,2S)-1-amino-2-methylcyclopropane-1-carboxylate. Final purification was done by prep-LCMS (XBridge C18 column, eluting with acetonitrile/aqueous 0.1% NH₄OH, at flow rate of 60 mL/min). LCMS calculated for C₂₈H₂₇D₇N₉O₄ (M+H)⁺: m/z=567.3; found 567.4. ¹H NMR (500 MHz, DMSO) δ 9.96 (s, 1H), 8.50 (s, 1H), 8.05 (s, 1H), 7.91 (d, J=8.6 Hz, 1H) 7.31 (s, 1H), 7.27 (s, 1H), 6.99 (d, J=8.2 Hz, 1H), 6.45 (s, 1H), 4.93-4.83 (m, 1H), 3.47 (s, 3H), 2.47-2.42 (m, 1H), 2.25-2.00 (m, 2H), 1.99-1.91 (m, 2H), 1.71-1.62 (m, 2H), 1.51-1.41 (m, 2H), 1.07-1.01 (m, 1H), 0.57-0.43 (m, 2H), 0.39-0.33 (m, 2H).

### Example 48. Methyl ((1R,3R)-3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

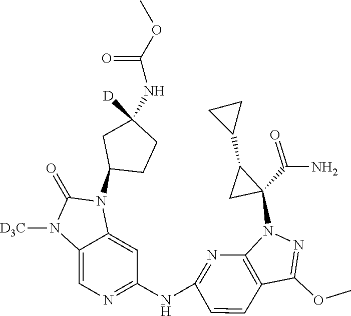 [SMILES: [2H]C([2H])([2H])n1c(=O)n([C@@H]2CC[C@@]([2H])(NC(=O)OC)C2)c2cc(Nc3ccc4c(OC)nn([C@@]5(C(N)=O)C[C@@H]5C5CC5)c4n3)ncc21]

### Step 1. Benzyl (1S,2R)-1-((tert-butoxycarbonyl)amino)-2-vinylcyclopropane-1-carboxylate

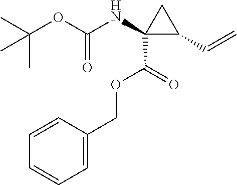

To a vial containing (1S,2R)-1-((tert-butoxycarbonyl)amino)-2-vinylcyclopropane-1-carboxylic acid (2.05 g, 9.0 mmol) in ACN (15 mL) was added K₂CO₃ (2.50 g, 18.0 mmol) and benzyl bromide (1.7 g, 9.9 mmol). The reaction vial was sealed and heated at 55° C. for 2 h. After cooling down to r.t., the mixture was concentrated to remove ACN. Water (30 mL) and DCM (30 mL) were added to the crude product and the two phases were separated. The water phase was extracted one more time with fresh DCM (30 mL). Organic phases were combined and concentrated in vacuo. The residue was purified by Biotage Isolera to afford the desired product. LCMS calculated for C₁₃H₁₆NO₂ (M-Boc+H)⁺: m/z=218.1; found 218.1

### Step 2 Benzyl (1R,2S)-2-amino-[1,1′-bi(cyclopropane)]-2-carboxylate

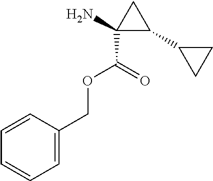

The title compound was prepared according to the procedures described in Example 45, Step 1, using benzyl (1S,2R)-1-((tert-butoxycarbonyl)amino)-2-vinylcyclopropane-1-carboxylate in place of methyl (1S,2R)-1-((tert-butoxycarbonyl)amino)-2-vinylcyclopropane-1-carboxylate. LCMS calculated for C₁₄H₁₈NO₂ (M+H)⁺: m/z=232.1; found 232.1

### Step 3. Benzyl (1R,2S)-2-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-[1,1′-bi(cyclopropane)]-2-carboxylate

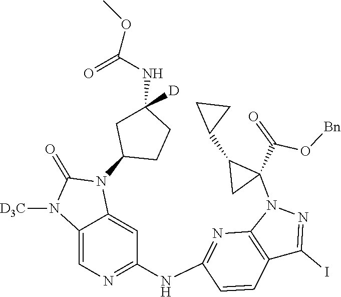 [SMILES: [2H]C([2H])([2H])n1c(=O)n([C@@H]2CC[C@@]([2H])(NC(=O)OC)C2)c2cc(Nc3ccc4c(I)nn([C@@]5(C(=O)OCc6ccccc6)C[C@@H]5C5CC5)c4n3)ncc21]

The title compound was prepared according to the procedures described in Example 54, Step 5-8, using benzyl (1R,2S)-2-amino-[1,1′-bi(cyclopropane)]-2-carboxylate in place of benzyl (1S,2S)-1-amino-2-methylcyclopropane-1-carboxylate. LCMS calculated for C₃₄H₃₂D₄N₈O₅(M+H)⁺: m/z=767.2; found 767.2

### Step 4. (1R,2S)-2-(3-Methoxy-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-[1,1′-bi(cyclopropane)]-2-carboxylic acid

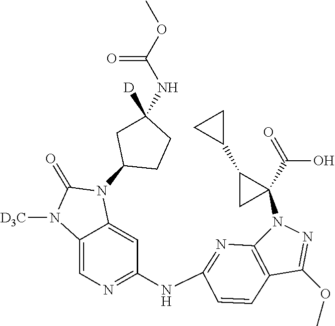 [SMILES: [2H]C([2H])([2H])n1c(=O)n([C@@H]2CC[C@@]([2H])(NC(=O)OC)C2)c2cc(Nc3ccc4c(OC)nn([C@@]5(C(=O)O)C[C@@H]5C5CC5)c4n3)ncc21]

To a vial containing benzyl (1R,2S)-2-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-[1,1′-bi(cyclopropane)]-2-carboxylate (60 mg, 78 mol) were added and Cs₂CO₃ (51 mg, 157 mol), 1,10-phenanthroline (28 mg, 157 mol), copper(I) iodide (15 mg, 78 mol), and methanol (1.6 mL, 39.1 mmol). The vial was sealed and heated at 100° C. for 2 h. After cooling to r.t., a premixed 1:1:1 MeOH/THF/1N NaOH water solution (2 mL) was added to the reaction mixture. The resulted mixture was heated at 50° C. for 1 hr. The reaction mixture was concentrated in vacuo and was added 2 mL of 1N NaOH solution and 2 mL of DCM. After extraction, water phase was neutralized by adding 1N HCl carefully until it turned cloudy (pH=4). The reaction mixture was extracted with DCM (10 mL) for 3 times. The organic phase was combined and washed with brine, dried over MgSO₄, and concentrated in vacuo. The resulted foamy solid was used in the next step without further purification. LCMS calculated for C₂₈H₂₉D₄N₈O₆(M+H)⁺: m/z=581.3; found 581.3.

### Step 5. Methyl ((1R,3R)-3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a vial containing (1R,2S)-2-(3-methoxy-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-[1,1′-bi(cyclopropane)]-2-carboxylic acid (10 mg, 17 mol) in DMF (0.2 mL) was added NH₃ solution (0.2 mL, 0.5 N in THF) and DIPEA (0.15 mL, 860 mol), followed by HATU (26 mg, 69 mol). The mixture was stirred at 55° C. for 1 h. The reaction mixture was diluted with DCM (10 mL) and washed with 1N NaOH solution (10 mL). The organic phase was concentrated in vacuo. The crude was purified by prep-LCMS (XBridge C18 column, eluting with a gradient of acetonitrile/water containing 0.1% TFA, at flow rate of 60 mL/min). LCMS calculated for C₂₈H₃₀D₄N₉O₅ (M+H)⁺: m/z=580.3; found 580.3. ¹H NMR (400 MHz, DMSO) δ 10.90-10.55 (bs, 1H), 8.18 (s, 1H), 7.99-7.93 (m, 2H), 7.56 (d, J=10.1 Hz, 2H), 6.92 (d, J=8.1 Hz, 1H), 6.83-6.77 (bs, 1H), 4.96-4.88 (m, 1H), 4.00 (s, 3H), 3.51 (s, 3H), 2.39 (dd, J=7.5, 13.5 Hz, 1H), 2.25-2.13 (m, 2H), 2.11-2.01 (m, 1H), 1.97-1.88 (m, 2H), 1.71-1.63 (m, 2H), 1.55-1.46 (m, 1H), 1.09-1.00 (m, 1H), 0.57-0.44 (m, 2H), 0.37-0.27 (m, 2H).

### Example 49. Methyl ((1R,3R)-3-(6-((1-((1S,2S)-1-carbamoyl-2-methylcyclopropyl)-4-(1-methyl-1H-pyrazol-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

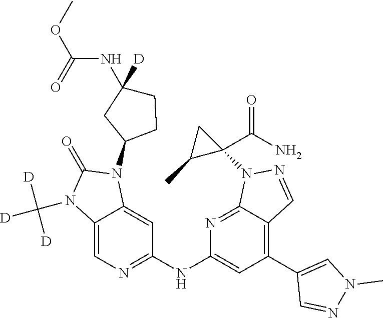 [SMILES: [2H]C([2H])([2H])n1c(=O)n([C@@H]2CC[C@@]([2H])(NC(=O)OC)C2)c2cc(Nc3cc(-c4cnn(C)c4)c4cnn([C@@]5(C(N)=O)C[C@@H]5C)c4n3)ncc21]

### Step 1. 4-Bromo-6-chloro-2-fluoronicotinic acid

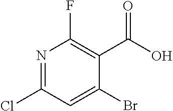 [SMILES: O=C(O)c1c(Br)cc(Cl)nc1F]

To a round-bottom flask containing 3-bromo-6-chloro-2-fluoropyridine (3.67 g, 17.4 mmol) was added tetrahydrofuran (53 mL) and subsequently sparged with N₂ gas for 5 minutes and cooled to −78° C. Lithium diisopropylamide (9.6 mL, 19.2 mmol, 2.0 M in THF) was added to the solution dropwise and stirred for 1 hour at −78° C. Dry CO₂ gas was bubbled through the reaction and the reaction was allowed to warm up to room temperature after 1.5 hours. The reaction was quenched with a 1M solution of HCl, extracted with DCM, and washed with brine. The organic layers were combined, dried over sodium sulfate, filtered, and the solvents were evaporated in vacuo. The crude material was purified by Biotage Isolera to give the product. Retention time=0.711 (Acquity UPLC BEH C18 1.7 μm 2.1×50 mm column, eluting with a gradient of 2% to 100% acetonitrile in water containing 0.05% TFA, at flow rate of 1 mL/min over 2 min.) ¹H NMR (400 MHz, DMSO) δ 14.55 (bs, 1H), 8.08 (s, 1H).

### Step 2. 4-Bromo-6-chloro-2-fluoronicotinamide

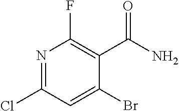 [SMILES: NC(=O)c1c(Br)cc(Cl)nc1F]

To a round-bottom flask containing 4-bromo-6-chloro-2-fluoronicotinic acid (1.24 g, 4.9 mmol) and dichloromethane (15 mL) cooled to 0° C. was added oxalyl chloride (4.9 mL, 9.7 mmol, 2.0 M in DCM) dropwise and stirred. Upon complete addition, the reaction was allowed to warm to room temperature and stirred for 1 hour. Subsequently the solvents were removed in vacuo. The crude residue was dissolved in 1,4-dioxane (5 mL), and this solution was added dropwise to a flask containing aqueous ammonium hydroxide (10 mL) cooled to 0° C. Upon complete addition, the reaction was warmed to room temperature and stirred for 15 min. The reaction was quenched with a saturated aqueous solution of ammonium chloride and extracted with ethyl acetate. The organic layers were combined, dried over sodium sulfate, filtered, and the solvents were evaporated in vacuo. The obtained crude product was used in the next step without further purification. Retention time=0.705 (Acquity UPLC BEH C18 1.7 μm 2.1×50 mm column, eluting with a gradient of 2% to 100% acetonitrile in water containing 0.05% TFA, at flow rate of 1 mL/min over 2 min.)

### Step 3. 4-Bromo-6-chloro-2-fluoronicotinonitrile

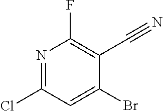 [SMILES: N#Cc1c(Br)cc(Cl)nc1F]

A round-bottom flask containing 4-bromo-6-chloro-2-fluoronicotinamide (1.22 g, 4.8 mmol), N,N-diisopropylethylamine (2.5 mL, 14.5 mmol), and tetrahydrofuran (10 mL) was cooled to 0° C. and to the reaction mixture was added trifluoroacetic anhydride (1.3 mL, 9.7 mmol) dropwise and allowed to stir at 0° C. for 1 hour. The reaction was quenched with a saturated solution of sodium bicarbonate and extracted with ethyl acetate. The organic layers were combined, dried over sodium sulfate, filtered, and the solvents were evaporated in vacuo. The crude material was purified by Biotage Isolera to give the product. Retention time=0.161 (Acquity UPLC BEH C18 1.7 μm 2.1×50 mm column, eluting with a gradient of 2% to 100% acetonitrile in water containing 0.05% TFA, at flow rate of 1 mL/min over 2 min.)

### Step 4. Benzyl (1S,2S)-1-(4-bromo-6-chloro-1H-pyrazolo[3,4-b]pyridin-1-yl)-2-methylcyclopropane-1-carboxylate

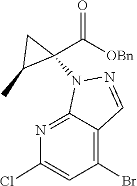 [SMILES: C[C@H]1C[C@]1(C(=O)OCc1ccccc1)n1ncc2c(Br)cc(Cl)nc21]

The title compound was prepared according to the procedures described in Example 24, Steps 4-6, with 4-bromo-6-chloro-2-fluoronicotinonitrile replacing 6-bromo-2-fluoronicotinonitrile and benzyl (1S,2S)-1-amino-2-methylcyclopropane-1-carboxylate (Example 54, Step 4) replacing isopropyl 1-amino-3-fluorocyclobutane-1-carboxylate in Step 4. LCMS calculated for C₁₁H₁₆BrClN₃O₂(M+H)⁺: m/z=420.0; found 420.0.

### Step 5. Benzyl (1S,2S)-1-(6-chloro-4-(1-methyl-1H-pyrazol-4-yl)-1H-pyrazolo[3,4-b]pyridin-1-yl)-2-methylcyclopropane-1-carboxylate

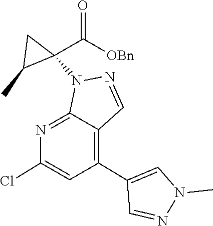

The title compound was prepared according to the procedures described in Example 1, Step 3 with benzyl (1S,2S)-1-(4-bromo-6-chloro-1H-pyrazolo[3,4-b]pyridin-1-yl)-2-methylcyclopropane-1-carboxylate replacing tert-butyl 1-(6-chloro-4-iodo-1H-pyrazolo[3,4-b]pyridin-1-yl)cyclopropane-1-carboxylate and with 1-methyl-4-(4,4,5,5-tetramethyl-1,3,2-dioxaborolan-2-yl)-1H-pyrazole replacing 2-(3,6-dihydro-2H-pyran-4-yl)-4,4,5,5-tetramethyl-1,3,2-dioxaborolane. LCMS calculated for C₂₂H₂₁ClN₅O₂(M+H)⁺: m/z=422.1; found 422.1.

### Step 6. Methyl ((1R,3R)-3-(6-((1-((1S,2S)-1-carbamoyl-2-methylcyclopropyl)-4-(1-methyl-1H-pyrazol-4-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

The title compound was prepared according to the procedures described in Example 14, Steps 3-4 with benzyl (1S,2S)-1-(6-chloro-4-(1-methyl-1H-pyrazol-4-yl)-1H-pyrazolo[3,4-b]pyridin-1-yl)-2-methylcyclopropane-1-carboxylate replacing methyl 1-(6-bromo-1H-pyrazolo[3,4-b]pyridin-1-yl)-3,3-difluorocyclobutane-1-carboxylate in Step 3, and final purification by prep-LCMS (XBridge C18 column, eluting with a gradient of 33% to 53% acetonitrile in water containing 0.1% NH₄OH, at flow rate of 60 mL/min.) LCMS calculated for C₂₉H₃₀D₄N₁₁O₄ (M+H)⁺: m/z=604.3; found 604.3. ¹H NMR (500 MHz, DMSO) δ 9.84 (s, 1H), 8.47 (s, 1H), 8.44 (s, 1H), 8.30 (s, 1H), 8.07 (s, 1H), 8.02 (s, 1H), 7.33 (s, 1H), 7.32 (s, 1H), 7.25 (s, 1H), 6.47 (s, 1H), 4.94-4.87 (m, 1H), 3.97 (s, 3H), 3.52 (s, 3H), 2.45 (dd, J=13.4, 7.5 Hz, 1H), 2.23 (ddd, J=12.6, 8.1, 4.7 Hz, 1H), 2.18-2.03 (m, 3H), 1.94 (dd, J=13.4, 9.3 Hz, 1H), 1.75 (dd, J=8.2, 4.8 Hz, 1H), 1.67 (ddd, J=12.5, 8.0, 8.0 Hz, 1H), 1.48 (dd, J=9.7, 4.7 Hz, 1H), 1.36 (d, J=6.3 Hz, 3H).

### Example 50. Methyl ((1R,3R)-3-(6-((1-((1S,2S)-1-carbamoyl-2-methylcyclopropyl)-4-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

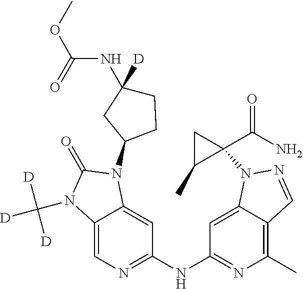 [SMILES: [2H]C([2H])([2H])n1c(=O)n([C@@H]2CC[C@@]([2H])(NC(=O)OC)C2)c2cc(Nc3cc4c(cnn4[C@@]4(C(N)=O)C[C@@H]4C)c(C)n3)ncc21]

The title compound was prepared according to the procedures described in Example 49, Steps 1-5, with trimethylboroxine replacing 1-methyl-4-(4,4,5,5-tetramethyl-1,3,2-dioxaborolan-2-yl)-1H-pyrazole in Step 5, and final purification by prep-LCMS (XBridge C₁₈ column, eluting with a gradient of 36% to 56% acetonitrile in water containing 0.1% NH₄OH, at flow rate of 60 mL/min over 5 min.) LCMS calculated for C₂₆H₂₈D₄N₉O₄(M+H)⁺: m/z=538.3; found 538.3. ¹H NMR (600 MHz, DMSO) δ 9.88 (s, 1H), 8.46 (s, 1H), 8.05 (s, 1H), 8.02 (s, 1H), 7.32 (s, 1H), 7.25 (s, 1H), 6.90 (s, 1H), 6.38 (s, 1H), 4.93-4.84 (m, 1H), 3.52 (s, 3H), 2.47 (s, 3H), 2.44 (dd, J=13.3, 7.5 Hz, 1H), 2.25-2.18 (m, 1H), 2.16-2.02 (m, 3H), 1.93 (dd, J=13.3, 9.3 Hz, 1H), 1.73 (dd, J=8.2, 4.8 Hz, 1H), 1.70-1.62 (m, 1H), 1.45 (dd, J=9.7, 4.7 Hz, 1H), 1.34 (d, J=6.3 Hz, 3H).

### Example 51. Methyl ((1R,3R)-3-(6-((1-((1S,2S)-1-carbamoyl-2-methylcyclopropyl)-3-(2,2,2-trifluoroethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

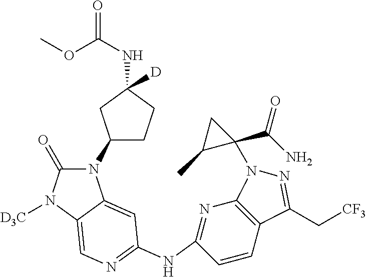 [SMILES: [2H]C([2H])([2H])n1c(=O)n([C@@H]2CC[C@@]([2H])(NC(=O)OC)C2)c2cc(Nc3ccc4c(CC(F)(F)F)nn([C@@]5(C(N)=O)C[C@@H]5C)c4n3)ncc21]

### Step 1. Benzyl (1S,2S)-1-(6-bromo-3-(2,2,2-trifluoroethyl)-1H-pyrazolo[3,4-b]pyridin-1-yl)-2-methylcyclopropane-1-carboxylate

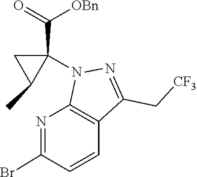 [SMILES: C[C@H]1C[C@]1(C(=O)OCc1ccccc1)n1nc(CC(F)(F)F)c2ccc(Br)nc21]

To a mixture of benzyl (1S,2S)-1-(6-bromo-3-formyl-1H-pyrazolo[3,4-b]pyridin-1-yl)-2-methylcyclopropane-1-carboxylate (400 mg, 0.966 mmol) in DMF (10 ml) was added 2,2-difluoro-2-(triphenylphosphonio)acetate (688 mg, 1.931 mmol), and the resulting mixture was heated to 60° C. for 2 hrs. The reaction mixture was then cooled to r.t. and a solution of tetrabutylammonium fluoride in THF (1M, 2.9 ml, 2.90 mmol) was added dropwise. The resulting solution was stirred at 60° C. for 45 min. The reaction was then cooled to r.t., quenched with water and diluted with EtOAc. The organic layer was extracted, washed with brine, dried with Na₂SO₄, filtered and concentrated. The crude material was purified by flash chromatography to afford the desired product (415 mg, 0.886 mmol, 92% yield). LCMS calculated for C₂₀H₁₈BrF₃N₃O₂ (M+H)⁺: m/z=468.1; found 468.1.

### Step 2. Methyl ((1R,3R)-3-(6-((1-((1S,2S)-1-carbamoyl-2-methylcyclopropyl)-3-(2,2,2-trifluoroethyl)-1H-pyrazolo[3,4-b]pyridin-6-yl)amino)-3-(methyl-d₃)-2-oxo-2,3-dihydro-H-imidazo[4,5-c]pyridin-1-yl)cyclopentyl-1-d)carbamate

The title compound was prepared according to the procedures described in Example 14, Step 3 and 4, with benzyl (1S,2S)-1-(6-bromo-3-(2,2,2-trifluoroethyl)-1H-pyrazolo[3,4-b]pyridin-1-yl)-2-methylcyclopropane-1-carboxylate replacing methyl 1-(6-bromo-1H-pyrazolo[3,4-b]pyridin-1-yl)-3,3-difluorocyclobutane-1-carboxylate. LCMS calculated for C₂₇H₂₇D₄F₃N₉O₄ (M+H)⁺: m/z=606.3; found 606.4. ¹H NMR (600 MHz, DMSO-d₆) δ 11.15 (s, 1H), 8.24 (s, 1H), 8.20 (d, J=8.7 Hz, 1H), 7.82 (s, 1H), 7.43 (s, 1H), 7.31 (s, 1H), 7.07 (d, J=8.7 Hz, 1H), 6.76 (s, 1H), 4.94 (m, 1H), 4.12-3.94 (m, 2H), 3.54 (s, 3H), 2.37 (m, 1H), 2.25-2.02 (m, 4H), 1.95 (m, 1H), 1.79 (m, 1H), 1.69-1.61 (m, 1H), 1.53-1.48 (m, 1H), 1.34 (d, J=6.3 Hz, 3H) ppm.

### Example 52. Methyl ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

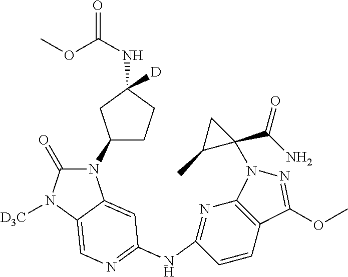 [SMILES: [2H]C([2H])([2H])n1c(=O)n([C@@H]2CC[C@@]([2H])(NC(=O)OC)C2)c2cc(Nc3ccc4c(OC)nn([C@@]5(C(N)=O)C[C@@H]5C)c4n3)ncc21]

### Step 1. (1S,2S)-1-(3-Methoxy-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid

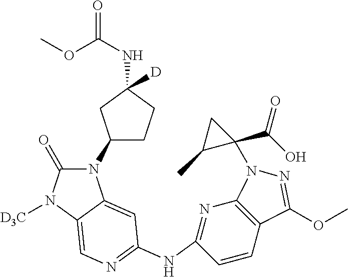 [SMILES: [2H]C([2H])([2H])n1c(=O)n([C@@H]2CC[C@@]([2H])(NC(=O)OC)C2)c2cc(Nc3ccc4c(OC)nn([C@@]5(C(=O)O)C[C@@H]5C)c4n3)ncc21]

A mixture of benzyl (1S,2S)-1-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (50 mg, 0.068 mmol), copper(I) iodide (2.6 mg, 0.014 mmol), 3,4,7,8-tetramethyl-1,10-phenanthroline (6.4 mg, 0.027 mmol), and cesium carbonate (44.0 mg, 0.135 mmol) was combined in methanol (1 mL) in a microwave vial and irradiated at 110° C. for 1 hr. The reaction was then cooled to r.t. and diluted with MeOH, filtered through celite and concentrated. The obtained crude was taken up in MeOH (0.5 mL), THF (0.5 mL), and water (0.5 mL), and lithium hydroxide (8 mg, 0.34 mmol) was added. The resulting mixture was stirred at 65° C. for 5 hrs. The reaction was cooled to r.t. and treated with aq. 1N HCl (0.675 mL). The solvent was evaporated in vacuo and the obtained product was used directly in the next step. LCMS calculated for C₂₆H₂₇D₄N₈O₆(M+H)⁺: m/z=555.3; found 555.3.

### Step 2. Methyl ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 14, Step 4 with (1S,2S)-1-(3-methoxy-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid replacing 3,3-difluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid. LCMS calculated for C₂₆H₂₈D₄N₉O₅(M+H)⁺: m/z=554.3; found 554.3. ¹H NMR (500 MHz, DMSO-d₆) δ 11.04 (s, 1H), 8.23 (s, 1H), 8.02 (d, J=8.6 Hz, 1H), 7.85-7.81 (m, 1H), 7.36-7.28 (m, 2H), 6.93 (d, J=8.6 Hz, 1H), 6.77 (s, 1H), 4.92 (m, 1H), 4.01 (s, 3H), 3.54 (s, 3H), 2.40-2.33 (m, 1H), 2.26-2.01 (m, 4H), 1.99-1.91 (m, 1H), 1.79-1.74 (m, 1H), 1.69-1.62 (m, 1H), 1.50-1.46 (m, 1H), 1.32 (d, J=6.3 Hz, 3H) ppm.

### Example 53. Methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

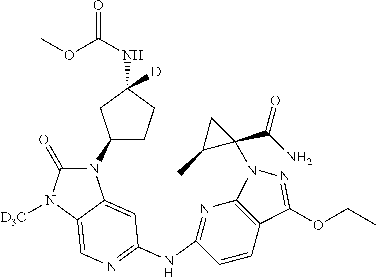 [SMILES: [2H]C([2H])([2H])n1c(=O)n([C@@H]2CC[C@@]([2H])(NC(=O)OC)C2)c2cc(Nc3ccc4c(OCC)nn([C@@]5(C(N)=O)C[C@@H]5C)c4n3)ncc21]

### Step 1. Dibenzyl (S)-2-methylcyclopropane-1,1-dicarboxylate

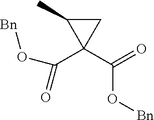 [SMILES: C[C@H]1CC1(C(=O)OCc1ccccc1)C(=O)OCc1ccccc1]

To a flask containing a suspension of NaH (7.03 g, 176 mmol) in DME (200 ml) was added dibenzyl malonate (17.6 ml, 70.3 mmol) in a dropwise manner at r.t. Then a solution of (R)-4-methyl-1,3,2-dioxathiolane 2,2-dioxide (11.7 g, 84 mmol) in DME (15 mL) was added slowly. The mixture was heated to reflux for 1 h before cooled to 0° C. After adding brine (100 ml) slowly, the mixture was extracted with Et₂O. The organic layers were combined, dried over Na₂SO₄, filtered, and the solvents were evaporated in vacuo. The crude material was purified by Biotage Isolera. LCMS calculated for: C₂₀H₂₁O₄(M+H)⁺: m/z=325.1; found 325.1.

### Step 2. (1R,2S)-1-((Benzyloxy)carbonyl)-2-methylcyclopropane-1-carboxylic acid

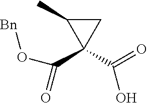 [SMILES: C[C@H]1C[C@@]1(C(=O)O)C(=O)OCc1ccccc1]

To a flask containing a solution of dibenzyl (S)-2-methylcyclopropane-1,1-dicarboxylate (19.14 g, 59.0 mmol) in t-BuOH (123 ml) and water (25 ml) was added dropwise NaOH solution (71 ml, 71 mmol, 1.0 M in water) at r.t. The mixture was stirred for 16 h and t-BuOH was evaporated in vacuo. The resulting aqueous layer was washed with ether and acidified with 1M HCl. The product was extracted with DCM and the combined organic layers were washed with brine, dried over Na₂SO₄, filtered, and the solvents were evaporated in vacuo. The crude material was directly used in the next step without further purification. LCMS calculated for: C₁₃H₁₅O₄(M+H)⁺: m/z=235.1; found 235.1.

### Step 3. Benzyl (1S,2S)-1-((tert-butoxycarbonyl)amino)-2-methylcyclopropane-1-carboxylate

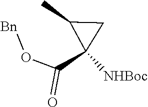 [SMILES: C[C@H]1C[C@@]1(NC(=O)OC(C)(C)C)C(=O)OCc1ccccc1]

To a flask containing (1R,2S)-1-((benzyloxy)carbonyl)-2-methylcyclopropane-1-carboxylic acid (13.44 g, 57.4 mmol) in toluene (200 mL) was added triethylamine (14.4 mL, 103 mmol) and diphenylphosphoryl azide (13.6 mL, 63.1 mmol). After heating at 50° C. for 20 min, the reaction mixture was cooled down to r.t., followed by the addition of t-BuOH (8.2 ml, 86 mmol) and DMAP (100 mg). Then the reaction mixture was heated to 90° C. for 16 h (note: nitrogen gas needs to be released). The reaction mixture was cooled down to r.t and concentrated in vacuo, and purified by Teledyne ISCO CombiFlash. LCMS calculated for C₁₇H₂₄NO₄ (M+H)⁺: m/z=306.2; found: 306.2.

### Step 4. Benzyl (1S,2S)-1-amino-2-methylcyclopropane-1-carboxylate

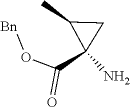

To a flask containing benzyl (1S,2S)-1-((tert-butoxycarbonyl)amino)-2-methylcyclopropane-1-carboxylate (2.9 g, 9.6 mmol) was added DCM (7 mL) and TFA (7 mL). The reaction mixture was stirred at r.t. for 1 h and concentrated in vacuo. The residue was dilute with DCM and neutralized with sat. NaHCO₃ aq. solution. The product was extracted with DCM. The organic layers were combined, dried over Na₂SO₄, filtered, and the solvents were evaporated in vacuo. The crude material was purified by Biotage Isolera. LCMS calculated for: C₁₂H₁₆NO₂ (M+H)⁺: m/z=206.1; found 206.1.

### Step 5. Benzyl (1S,2S)-1-((6-bromo-3-cyanopyridin-2-yl)amino)-2-methylcyclopropane-1-carboxylate

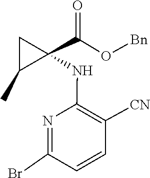 [SMILES: C[C@H]1C[C@@]1(Nc1nc(Br)ccc1C#N)C(=O)OCc1ccccc1]

To a vial containing 6-bromo-2-fluoronicotinonitrile (1.86 g, 9.24 mmol) and benzyl (1S,2S)-1-amino-2-methylcyclopropane-1-carboxylate (1.58 g, 7.7 mmol) in DMSO (4 mL) was added DIPEA (1.75 mL, 10 mmol). After heating at 65° C. for 20 h, the reaction mixture was directly purified by Biotage Isolera to give the desired product as light-yellow solid. LCMS calculated for C₁₈H₁₇BrN₃O₂(M+H)⁺: m/z=386.0; found 386.0.

### Step 6. Benzyl (1S,2S)-1-(3-amino-6-bromo-1H-pyrazolo[3,4-b]pyridin-1-yl)-2-methylcyclopropane-1-carboxylate

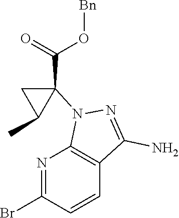 [SMILES: C[C@H]1C[C@]1(C(=O)OCc1ccccc1)n1nc(N)c2ccc(Br)nc21]

To a vial containing benzyl (1S,2S)-1-((6-bromo-3-cyanopyridin-2-yl)amino)-2-methylcyclopropane-1-carboxylate (3.11 g, 8.05 mmol) in THF (11 mL) and DMF (22 mL) at 0° C. was slowly added LHMDS (10.5 mL, 10.5 mmol, 1.0 M in THF). After stirring at 0° C. for 10 min, (aminooxy)diphenylphosphine oxide (2.8 g, 12.08 mmol) was added to the reaction mixture. The reaction was allowed to warm up to r.t. with monitoring of stirring rate. After stirring at r.t. for 1 h, the reaction mixture was treated with 1 M HCl solution (19 mL) and stirred for 10 min. The reaction mixture was neutralized with sat. NaHCO₃ solution, followed by extraction with diethyl ether (50 mL) for 3 times. The organic phases were combined and washed with brine, dried over MgSO₄ and concentrated in vacuo. The crude material was purified by Biotage Isolera to give the desired product. LCMS calculated for C₁₈H₁₈BrN₄O₂(M+H)⁺: m/z=401.0; found 401.0.

### Step 7. Benzyl (1S,2S)-1-(3-(bis(tert-butoxycarbonyl)amino)-6-bromo-1H-pyrazolo[3,4-b]pyridin-1-yl)-2-methylcyclopropane-1-carboxylate

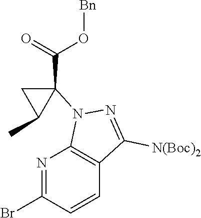

To a vial containing benzyl (1S,2S)-1-(3-amino-6-bromo-1H-pyrazolo[3,4-b]pyridin-1-yl)-2-methylcyclopropane-1-carboxylate (246 mg, 0.61 mmol) in THF (4 mL) was added triethylamine (0.13 mL, 0.92 mmol) and DMAP (7.5 mg, 0.061 mmol). Di-tert-butyl dicarbonate (0.31 mL, 1.35 mmol) was added in one portion and the reaction mixture was stirred at r.t. for 1 h. The reaction mixture was concentrated in vacuo and purified by Biotage Isolera. LCMS calculated for C₂₈H₃₄BrN₄O₆(M+H)⁺: m/z=601.2; found 601.2.

### Step 8. Benzyl (1S,2S)-1-(3-(bis(tert-butoxycarbonyl)amino)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

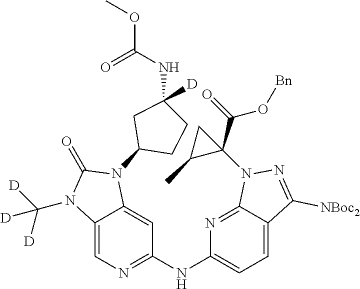

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (185 mg, 0.57 mmol, Intermediate A), benzyl (1S,2S)-1-(3-(bis(tert-butoxycarbonyl)amino)-6-bromo-1H-pyrazolo[3,4-b]pyridin-1-yl)-2-methylcyclopropane-1-carboxylate (287 mg, 0.45 mmol), tris(dibenzylideneacetone)-dipalladium(0) (65 mg, 0.072 mmol), 4,5-bis(diphenylphosphino)-9,9-dimethylxanthene (83 mg, 0.14 mmol), and cesium carbonate (311 mg, 0.95 mmol) was evacuated and backfilled with nitrogen three times, followed by the addition of 1,4-dioxane (4 mL). The vial was sealed and heated to 100° C. for 2 h. After cooling to r.t., the mixture was filtered and concentrated in vacuo. The crude material was purified by Biotage Isolera to give the desired product. LCMS calculated for C₄₂1-H₄₈D₄N₉O₉(M+H)⁺: m/z=830.4; found 830.4.

### Step 9. Benzyl (1S,2S)-1-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

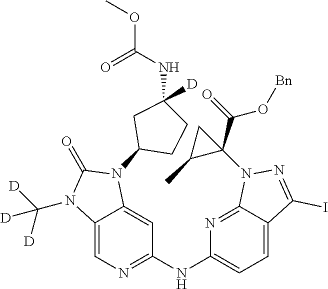 [SMILES: [2H]C([2H])([2H])n1c(=O)n([C@@H]2CC[C@@]([2H])(NC(=O)OC)C2)c2cc(Nc3ccc4c(I)nn([C@@]5(C(=O)OCc6ccccc6)C[C@@H]5C)c4n3)ncc21]

To a flask containing benzyl (1S,2S)-1-(3-(bis(tert-butoxycarbonyl)amino)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (165 mg, 0.2 mmol) was added DCM (2 mL) and TFA (2 mL). The reaction mixture was stirred at r.t. for 2 h and concentrated in vacuo. The residue was dilute with DCM and neutralized with sat. NaHCO₃ aq. solution. The product was extracted with DCM. The organic layers were combined, dried over Na₂SO₄, filtered, and the solvents were evaporated in vacuo. The residue was dissolved in MeCN (3 mL) and KI (33 mg, 0.2 mmol) and I₂ (0.1 g, 0.4 mmol) were added. The mixture was cooled to 0° C. and tert-butyl nitrite (0.094 mL, 0.8 mmol) was added. The vial was heated to 60° C. for 2 h. After cooling to r.t., the mixture was quenched with sat. Na₂S₂O₃ aq. solution followed by extraction with DCM. The organic phases were combined and washed with brine, dried over MgSO₄ and concentrated in vacuo. The crude material was purified by Biotage Isolera to give the desired product. LCMS calculated for C₃₂H₃₀D₄IN₈O₅(M+H)⁺: m/z=741.2; found 741.2.

### Step 10. (1S,2S)-1-(3-Ethoxy-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid

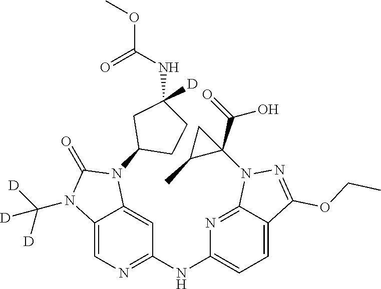 [SMILES: [2H]C([2H])([2H])n1c(=O)n([C@@H]2CC[C@@]([2H])(NC(=O)OC)C2)c2cc(Nc3ccc4c(OCC)nn([C@@]5(C(=O)O)C[C@@H]5C)c4n3)ncc21]

A mixture of benzyl (1S,2S)-1-(3-iodo-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (500.0 mg, 0.675 mmol), copper(I) iodide (25.7 mg, 0.135 mmol), 3,4,7,8-tetramethyl-1,10-phenanthroline (63.8 mg, 0.270 mmol) and Cs₂CO₃ (330.0 mg, 1.013 mmol) in EtOH (6.8 mL) was irradiated in a microwave vial at 110° C. for 1 hr. The reaction mixture was then transferred to a flask and solvent was evaporated. The obtained crude was dissolved in 7 mL MeOH and Cs₂CO₃ (330.0 mg, 1.013 mmol) was added. The reaction mixture was irradiated in a microwave vial at 120° C. for 2.5 hrs. The reaction was then cooled to r.t. and diluted with MeOH, filtered through celite, and concentrated. The crude was taken up in MeOH (2 mL), THF (2 mL), and water (2 mL), and lithium hydroxide (243 mg, 10.13 mmol) was added. The resulting mixture was stirred at 65° C. for 7 hrs. The reaction mixture was cooled to r.t. and treated with aq. 1N HCl (10.1 mL). Solvent was removed and the obtained product was used in the next step.

### Step 11. Methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)caramate

A mixture of (1S,2S)-1-(3-ethoxy-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid (768.0 mg, 1.351 mmol), HATU (2054 mg, 5.40 mmol), ammonia in dioxane (0.4 M, 27.0 ml, 10.8 mmol), and N,N-diisopropylethylamine (1.18 ml, 6.75 mmol) in DMF (6.8 ml) was stirred at 60° C. After 1.5 hrs, volatile solvent was removed and the crude was diluted with water and DCM. The organic layer was extracted with DCM, washed with aq. NaOH (1M), dried over Na₂SO₄, and concentrated. The crude was purified by Combi-Flash (EtOAc/Hex then MeOH/DCM), followed by prep-LCMS (XBridge C18 column, acetonitrile/water containing 0.1% TFA, at flow rate of 60 mL/min). LCMS calculated for C₂₇H₃₀D₄N₉O₅ (M+H)⁺: m/z=568.3; found 568.4. ¹H NMR (600 MHz, DMSO-d6) δ 10.95 (s, 1H), 8.22 (s, 1H), 8.00 (d, J=8.6 Hz, 1H), 7.84 (s, 1H), 7.37-7.29 (m, 2H), 6.92 (d, J=8.6 Hz, 1H), 6.75 (s, 1H), 4.91 (m, 1H), 4.44-4.33 (m, 2H), 3.53 (s, 3H), 2.40-2.34 (m, 1H), 2.25-2.01 (m, 4H), 1.97-1.90 (m, 1H), 1.77-1.72 (m, 1H), 1.68-1.61 (m, 1H), 1.49-1.44 (m, 1H), 1.41 (t, J=7.1 Hz, 3H), 1.32 (d, J=6.3 Hz, 3H) ppm.

### Example 54. Methyl ((1R,3R)-3-(6-((1-((1S,2S)-1-carbamoyl-2-methylcyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

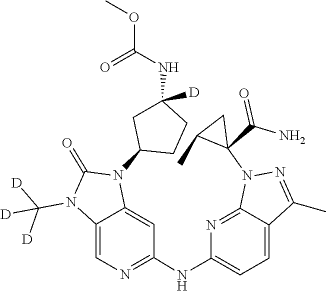 [SMILES: [2H]C([2H])([2H])n1c(=O)n([C@@H]2CC[C@@]([2H])(NC(=O)OC)C2)c2cc(Nc3ccc4c(C)nn([C@@]5(C(N)=O)C[C@@H]5C)c4n3)ncc21]

### Step 1. Dibenzyl (S)-2-methylcyclopropane-1,1-dicarboxylate

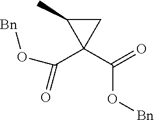 [SMILES: C[C@H]1CC1(C(=O)OCc1ccccc1)C(=O)OCc1ccccc1]

To a flask containing a suspension of NaH (7.03 g, 176 mmol) in DME (200 ml) was added dibenzyl malonate (17.6 ml, 70.3 mmol) in a dropwise manner at r.t. Then a solution of (R)-4-methyl-1,3,2-dioxathiolane 2,2-dioxide (11.7 g, 84 mmol) in DME (15 mL) was added slowly. The mixture was heated to reflux for 1 h before cooled to 0° C. After adding brine (100 ml) slowly, the mixture was extracted with Et₂O. The organic layers were combined, dried over Na₂SO₄, filtered, and the solvents were evaporated in vacuo. The crude material was purified by Biotage Isolera. LCMS calculated for: C₂₀H₂₁O₄(M+H)⁺: m/z=325.1; found 325.1.

### Step 2. (1R,2S)-1-((Benzyloxy)carbonyl)-2-methylcyclopropane-1-carboxylic acid

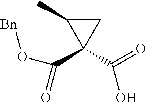 [SMILES: C[C@H]1C[C@@]1(C(=O)O)C(=O)OCc1ccccc1]

To a flask containing a solution of dibenzyl (S)-2-methylcyclopropane-1,1-dicarboxylate (19.14 g, 59.0 mmol) in t-BuOH (123 ml) and water (25 ml) was added dropwise NaOH solution (71 ml, 71 mmol, 1.0 M in water) at r.t. The mixture was stirred for 16 h and t-BuOH was evaporated in vacuo. The resulting aqueous layer was washed with ether and acidified with 1M HCl. The product was extracted with DCM and the combined organic layers were washed with brine, dried over Na₂SO₄, filtered, and the solvents were evaporated in vacuo. The crude material was directly used in the next step without further purification. LCMS calculated for: C₁₃H₁₅O₄(M+H)⁺: m/z=235.1; found 235.1.

### Step 3. Benzyl (1S,2S)-1-((tert-butoxycarbonyl)amino)-2-methylcyclopropane-1-carboxylate

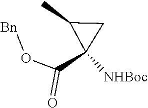 [SMILES: C[C@H]1C[C@@]1(NC(=O)OC(C)(C)C)C(=O)OCc1ccccc1]

To a flask containing (1R,2S)-1-((benzyloxy)carbonyl)-2-methylcyclopropane-1-carboxylic acid (13.44 g, 57.4 mmol) in toluene (200 mL) was added triethylamine (14.4 mL, 103 mmol) and diphenylphosphoryl azide (13.6 mL, 63.1 mmol). After heating at 50° C. for 20 min, the reaction mixture was cooled down to r.t., followed by the addition of t-BuOH (8.2 ml, 86 mmol) and DMAP (100 mg). Then the reaction mixture was heated to 90° C. for 16 h (note: nitrogen gas needs to be released). The reaction mixture was cooled down to r.t and concentrated in vacuo, and purified by Teledyne ISCO CombiFlash. LCMS calculated for C₁₇H₂₄NO₄ (M+H)⁺: m/z=306.2; found: 306.2.

### Step 4. Benzyl (1S,2S)-1-amino-2-methylcyclopropane-1-carboxylate

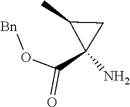

To a flask containing benzyl (1S,2S)-1-((tert-butoxycarbonyl)amino)-2-methylcyclopropane-1-carboxylate (2.9 g, 9.6 mmol) was added DCM (7 mL) and TFA (7 mL). The reaction mixture was stirred at r.t. for 1 h and concentrated in vacuo. The residue was dilute with DCM and neutralized with sat. NaHCO₃ aq. solution. The product was extracted with DCM. The organic layers were combined, dried over Na₂SO₄, filtered, and the solvents were evaporated in vacuo. The crude material was purified by Biotage Isolera. LCMS calculated for: C₁₂H₁₆NO₂ (M+H)⁺: m/z=206.1; found 206.1.

### Step 5. Benzyl (1S,2S)-1-((6-bromo-3-cyanopyridin-2-yl)amino)-2-methylcyclopropane-1-carboxylate

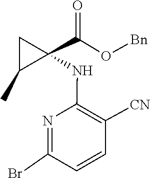 [SMILES: C[C@H]1C[C@@]1(Nc1nc(Br)ccc1C#N)C(=O)OCc1ccccc1]

To a vial containing 6-bromo-2-fluoronicotinonitrile (1.86 g, 9.24 mmol) and benzyl (1S,2S)-1-amino-2-methylcyclopropane-1-carboxylate (1.58 g, 7.7 mmol) in DMSO (4 mL) was added DIPEA (1.75 mL, 10 mmol). After heating at 65° C. for 20 h, the reaction mixture was directly purified by Biotage Isolera to give the desired product as light-yellow solid. LCMS calculated for C₁₈H₁BrN₃O₂(M+H)⁺: m/z=386.0; found 386.0.

### Step 6. Benzyl (1S,2S)-1-(3-amino-6-bromo-1H-pyrazolo[3,4-b]pyridin-1-yl)-2-methylcyclopropane-1-carboxylate

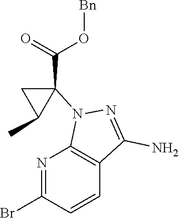 [SMILES: C[C@H]1C[C@]1(C(=O)OCc1ccccc1)n1nc(N)c2ccc(Br)nc21]

To a vial containing benzyl (1S,2S)-1-((6-bromo-3-cyanopyridin-2-yl)amino)-2-methylcyclopropane-1-carboxylate (3.11 g, 8.05 mmol) in THF (11 mL) and DMF (22 mL) at 0° C. was slowly added LHMDS (10.5 mL, 10.5 mmol, 1.0 M in THF). After stirring at 0° C. for 10 min, (aminooxy)diphenylphosphine oxide (2.8 g, 12.08 mmol) was added to the reaction mixture. The reaction was allowed to warm up to r.t. with monitoring of stirring rate. After stirring at r.t. for 1 h, the reaction mixture was treated with 1 M HCl solution (19 mL) and stirred for 10 min. The reaction mixture was neutralized with sat. NaHCO₃ solution, followed by extraction with diethyl ether (50 mL) for 3 times. The organic phases were combined and washed with brine, dried over MgSO₄ and concentrated in vacuo. The crude material was purified by Biotage Isolera to give the desired product. LCMS calculated for C₁₈H₁₈BrN₄O₂(M+H)⁺: m/z=401.0; found 401.0.

### Step 7. Benzyl (1S,2S)-1-(3-(bis(tert-butoxycarbonyl)amino)-6-bromo-1H-pyrazolo[3,4-b]pyridin-1-yl)-2-methylcyclopropane-1-carboxylate

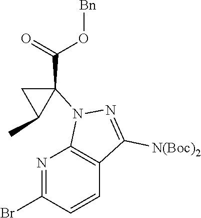

To a vial containing benzyl (1S,2S)-1-(3-amino-6-bromo-1H-pyrazolo[3,4-b]pyridin-1-yl)-2-methylcyclopropane-1-carboxylate (246 mg, 0.61 mmol) in THF (4 mL) was added triethylamine (0.13 mL, 0.92 mmol) and DMAP (7.5 mg, 0.061 mmol). Di-tert-butyl dicarbonate (0.31 mL, 1.35 mmol) was added in one portion and the reaction mixture was stirred at r.t. for 1 h. The reaction mixture was concentrated in vacuo and purified by Biotage Isolera. LCMS calculated for C₂₈H₃₄BrN₄O₆(M+H)⁺: m/z=601.2; found 601.2.

### Step 8. Benzyl (1S,2S)-1-(3-(bis(tert-butoxycarbonyl)amino)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

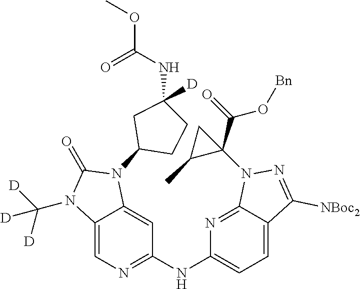

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (185 mg, 0.57 mmol, Intermediate A), benzyl (1S,2S)-1-(3-(bis(tert-butoxycarbonyl)amino)-6-bromo-1H-pyrazolo[3,4-b]pyridin-1-yl)-2-methylcyclopropane-1-carboxylate (287 mg, 0.45 mmol), tris(dibenzylideneacetone)-dipalladium(0) (65 mg, 0.072 mmol), 4,5-bis(diphenylphosphino)-9,9-dimethylxanthene (83 mg, 0.14 mmol), and cesium carbonate (311 mg, 0.95 mmol) was evacuated and backfilled with nitrogen three times, followed by the addition of 1,4-dioxane (4 mL). The vial was sealed and heated to 100° C. for 2 h. After cooling to r.t., the mixture was filtered and concentrated in vacuo. The crude material was purified by Biotage Isolera to give the desired product. LCMS calculated for C₄₂1-H₄₈D₄N₉O₉(M+H)⁺: m/z=830.4; found 830.4.

### Step 9. Benzyl (1S,2S)-1-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

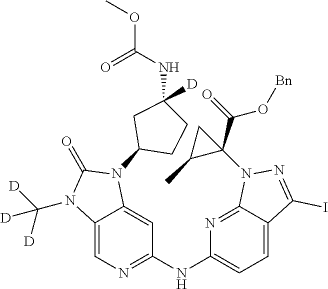 [SMILES: [2H]C([2H])([2H])n1c(=O)n([C@@H]2CC[C@@]([2H])(NC(=O)OC)C2)c2cc(Nc3ccc4c(I)nn([C@@]5(C(=O)OCc6ccccc6)C[C@@H]5C)c4n3)ncc21]

To a flask containing benzyl (1S,2S)-1-(3-(bis(tert-butoxycarbonyl)amino)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (165 mg, 0.2 mmol) was added DCM (2 mL) and TFA (2 mL). The reaction mixture was stirred at r.t. for 2 h and concentrated in vacuo. The residue was dilute with DCM and neutralized with sat. NaHCO₃ aq. solution. The product was extracted with DCM. The organic layers were combined, dried over Na₂SO₄, filtered, and the solvents were evaporated in vacuo. The residue was dissolved in MeCN (3 mL) and KI (33 mg, 0.2 mmol) and I₂ (0.1 g, 0.4 mmol) were added. The mixture was cooled to 0° C. and tert-butyl nitrite (0.094 mL, 0.8 mmol) was added. The vial was heated to 60° C. for 2 h. After cooling to r.t., the mixture was quenched with sat. Na₂S₂O₃ aq. solution followed by extraction with DCM. The organic phases were combined and washed with brine, dried over MgSO₄ and concentrated in vacuo. The crude material was purified by Biotage Isolera to give the desired product. LCMS calculated for C₃₂H₃₀D₄IN₈O₅(M+H)⁺: m/z=741.2; found 741.2.

### Step 10. Benzyl (1S,2S)-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-methyl-1H-pyrazolo[3,4-b]pyridin-1-yl)-2-methylcyclopropane-1-carboxylate

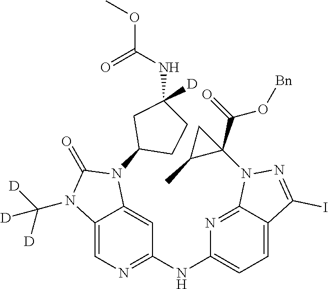 [SMILES: [2H]C([2H])([2H])n1c(=O)n([C@@H]2CC[C@@]([2H])(NC(=O)OC)C2)c2cc(Nc3ccc4c(I)nn([C@@]5(C(=O)OCc6ccccc6)C[C@@H]5C)c4n3)ncc21]

A vial containing benzyl (1S,2S)-1-(3-iodo-6-((1-((1R,3R)-3-((methoxycarbonyl)amino)-cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (55 mg, 0.074 mmol), tris(dibenzylideneacetone)-dipalladium(0) (7 mg, 0.0074 mmol), 1,2,3,4,5-pentaphenyl-1′-(di-tert-butylphosphino)ferrocene (11 mg, 0.015 mmol), and methylzinc chloride (0.15 mL, 0.3 mmol, 2 M in THF) was evacuated and backfilled with nitrogen three times, followed by the addition of THF (2 mL). The vial was sealed and heated to 50° C. for 2 h. After cooling to r.t., the mixture was quenched with water and filtered, followed by extraction with EtOAc 3 times. The organic phases were combined and washed with brine, dried over MgSO₄ and concentrated in vacuo. The crude material was purified by Biotage Isolera to give the desired product. LCMS calculated for C₃₃H₃₃D₄N₈O₅(M+H)⁺: m/z=629.3; found 629.3.

### Step 11. Methyl ((1R,3R)-3-(6-((1-((1S,2S)-1-carbamoyl-2-methylcyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a reaction vial containing benzyl (1S,2S)-1-(6-((1-((1R,3R)-3-((methoxycarbonyl)amino)-cyclopentyl-3-d)-3-(methyl-d₃)-2-oxo-2,3-dihydro-1H-imidazo[4,5-c]pyridin-6-yl)amino)-3-methyl-1H-pyrazolo[3,4-b]pyridin-1-yl)-2-methylcyclopropane-1-carboxylate (42 mg, 0.07 mmol) in THF (1 mL), MeOH (1 mL) and water (1 mL) was added lithium hydroxide monohydrate (14 mg, 0.33 mmol). The reaction mixture was heated to 50° C. and allowed to stir for 4 h, after which the reaction was quenched with HCl (1 mL, 1 mmol, 1 M in water) and extracted with 3:1 chloroform:isopropanol. The organic layers were combined, dried over sodium sulfate, filtered, and the solvents were evaporated in vacuo. The crude material was added to a reaction vial containing HATU (31 mg, 0.08 mmol) and DIPEA (0.014 mL, 0.08 mmol) in DMF (0.2 mL) and THF (0.6 mL). The reaction vial was cooled to 0° C. before ammonia (0.25 mL, 0.1 mmol, 0.4 M in dioxane) was added to this reaction mixture, and it was then sealed and stirred at 50° C. for 2 h. Upon completion, the mixture was diluted with EtOAc and washed with 1N NaOH aq. solution. The organic phase was dried over MgSO₄ and concentrated in vacuo. The reaction residue was diluted with MeCN and purified by prep-LCMS (XBridge C18 column, acetonitrile/water containing 0.1% TFA, at flow rate of 60 mL/min). LCMS calculated for: C₂₆H₂₈D₄N₉O₄(M+H)⁺: m/z=538.3; found 538.3. ¹H NMR (600 MHz, DMSO) δ 11.06 (s, 1H), 8.22 (s, 1H), 8.14 (d, J=8.6 Hz, 1H), 7.78 (s, 1H), 7.37 (s, 1H), 7.32 (s, 1H), 6.98 (d, J=8.6 Hz, 1H), 6.75 (s, 1H), 4.96-4.91 (m, 1H), 3.54 (s, 3H), 2.46 (s, 3H), 2.36 (dd, J=13.6, 7.6 Hz, 1H), 2.21 (m, 1H), 2.17-2.11 (m, 2H), 2.09-2.03 (m, 1H), 1.95 (dd, J=13.6, 9.5 Hz, 1H), 1.77 (dd, J=8.0, 4.9 Hz, 1H), 1.68-1.63 (m, 1H), 1.42 (dd, J=9.7, 4.9 Hz, 1H), 1.33 (d, J=6.3 Hz, 3H).

### Example 55. Methyl ((1R,3R)-3-(6-((1-((1S,2S)-1-carbamoyl-2-methylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

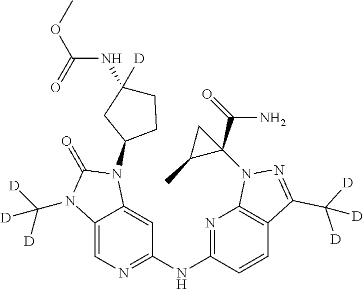 [SMILES: [2H]C([2H])([2H])c1nn([C@@]2(C(N)=O)C[C@@H]2C)c2nc(Nc3cc4c(cn3)n(C([2H])([2H])[2H])c(=O)n4[C@@H]3CC[C@@]([2H])(NC(=O)OC)C3)ccc12]

The title compound was prepared according to the procedures described in Example 54, Step 10-11, with methyl-d₃-zinc chloride replacing methylzinc chloride. LCMS calculated for: C₂₆H₂₅D₇N₉O₄(M+H)⁺: m/z=541.3; found 541.3. ¹H NMR (500 MHz, DMSO) δ 11.06 (s, 1H), 8.22 (s, 1H), 8.15 (d, J=8.6 Hz, 1H), 7.78 (s, 1H), 7.36 (s, 1H), 7.32 (s, 1H), 6.98 (d, J=8.6 Hz, 1H), 6.74 (s, 1H), 4.97-4.90 (m, 1H), 3.54 (s, 3H), 2.37 (dd, J=13.6, 7.7 Hz, 1H), 2.26-2.01 (m, 4H), 1.95 (dd, J=13.6, 9.5 Hz, 1H), 1.77 (dd, J=8.0, 4.9 Hz, 1H), 1.68-1.63 (m, 1H), 1.43 (dd, J=9.7, 4.9 Hz, 1H), 1.33 (d, J=6.3 Hz, 3H).

### Example 56. Methyl ((1R,3R)-3-(6-((1-((1S,2S)-1-carbamoyl-2-methylcyclopropyl)-3,4-dimethyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

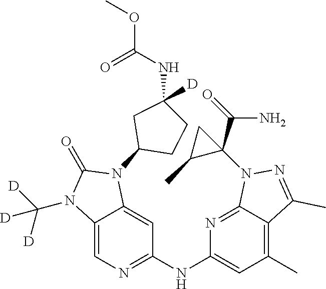 [SMILES: [2H]C([2H])([2H])n1c(=O)n([C@@H]2CC[C@@]([2H])(NC(=O)OC)C2)c2cc(Nc3cc(C)c4c(C)nn([C@@]5(C(N)=O)C[C@@H]5C)c4n3)ncc21]

### Step 1. Benzyl (1S,2S)-1-(3-amino-4-bromo-1H-pyrazolo[3,4-b]pyridin-1-yl)-2-methylcyclopropane-1-carboxylate

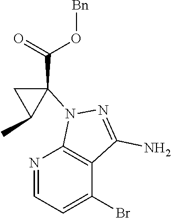 [SMILES: C[C@H]1C[C@]1(C(=O)OCc1ccccc1)n1nc(N)c2c(Br)ccnc21]

The title compound was prepared according to the procedures described in Example 54, Step 5-6, with 4-bromo-2-fluoronicotinonitrile replacing 6-bromo-2-fluoronicotinonitrile. LCMS calculated for C₁₁H₁₈BrN₄O₂(M+H)⁺: m/z=401.0; found 401.0.

### Step 2. Benzyl (1S,2S)-1-(4-bromo-3-iodo-1H-pyrazolo[3,4-b]pyridin-1-yl)-2-methylcyclopropane-1-carboxylate

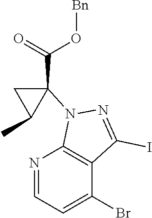 [SMILES: C[C@H]1C[C@]1(C(=O)OCc1ccccc1)n1nc(I)c2c(Br)ccnc21]

To a flask containing benzyl (1S,2S)-1-(3-amino-4-bromo-1H-pyrazolo[3,4-b]pyridin-1-yl)-2-methylcyclopropane-1-carboxylate (587 mg, 1.46 mmol) were added MeCN (4 mL), KI (243 mg, 1.46 mmol), and I₂ (743 mg, 2.93 mmol). The mixture was cooled to 0° C. and tert-butyl nitrite (0.70 mL, 5.85 mmol) was added. The reaction mixture was stirred at r.t. for 1 h. The mixture was quenched with sat. Na₂S₂O₃ aq. solution followed by the extraction with DCM. The organic phases were combined, washed with brine, dried over MgSO₄, and concentrated in vacuo. The crude material was purified by Biotage Isolera to give the desired product. LCMS calculated for C₁₈H₁₆BrIN₃O₂ (M+H)⁺: m/z=511.9; found 512.0.

### Step 3. Benzyl (1S,2S)-1-(3,4-dimethyl-1H-pyrazolo[3,4-b]pyridin-1-yl)-2-methylcyclopropane-1-carboxylate

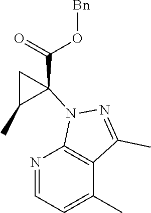

A vial containing benzyl (1S,2S)-1-(4-bromo-3-iodo-1H-pyrazolo[3,4-b]pyridin-1-yl)-2-methylcyclopropane-1-carboxylate (538 mg, 1.05 mmol), tris(dibenzylideneacetone)-dipalladium(0) (96 mg, 0.11 mmol), 1,2,3,4,5-pentaphenyl-1′-(di-tert-butylphosphino)ferrocene (151 mg, 0.21 mmol), and methylzinc chloride (1.31 mL, 2.63 mmol, 2 M in THF) was evacuated and backfilled with nitrogen three times, followed by the addition of THF (5 mL). The vial was sealed and stirred at r.t. for 90 min. The mixture was quenched with water and filtered, followed by the extraction with EtOAc for 3 times. The organic phases were combined, washed with brine, dried over MgSO₄, and concentrated in vacuo. The crude material was purified by Biotage Isolera to give the desired product. LCMS calculated for C₂₀H₂₂N₃O₂ (M+H)⁺: m/z=336.2; found 336.2.

### Step 4. 1-((1S,2S)-1-((Benzyloxy)carbonyl)-2-methylcyclopropyl)-3,4-dimethyl-1H-pyrazolo[3,4-b]pyridine 7-oxide

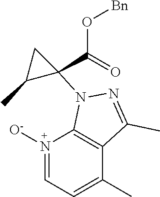

To a cooled vial containing benzyl (1S,2S)-1-(3,4-dimethyl-1H-pyrazolo[3,4-b]pyridin-1-yl)-2-methylcyclopropane-1-carboxylate (85 mg, 0.25 mmol) and urea hydrogen peroxide (74 mg, 0.79 mmol) in DCM (3 mL) was added trifluoroacetic anhydride (0.11 mL, 0.76 mmol) dropwise. The mixture was warmed to r.t. and stirred for 2 h before diluted with DCM and washed with sat. NaHCO₃ aq. solution. The organic phase was dried over MgSO₄ and concentrated in vacuo. The crude material was purified by Biotage Isolera to give the desired product. LCMS calculated for C₂₀H₂₂N₃O₃ (M+H)⁺: m/z=352.2; found 352.2.

### Step 5. Benzyl (1S,2S)-1-(6-chloro-3,4-dimethyl-1H-pyrazolo[3,4-b]pyridin-1-yl)-2-methylcyclopropane-1-carboxylate

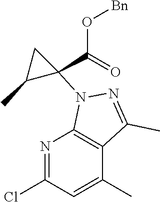

To a vial containing 1-((1S,2S)-1-((benzyloxy)carbonyl)-2-methylcyclopropyl)-3,4-dimethyl-1H-pyrazolo[3,4-b]pyridine 7-oxide (15 mg, 0.043 mmol) was added phosphoryl chloride (0.5 mL) and it was sealed and stirred at 100° C. for 1 h. Upon completion, the mixture was diluted with DCM and quenched with sat. NaHCO₃ aq. solution. The organic phase was dried over MgSO₄ and concentrated in vacuo. The crude material was used without further purification. LCMS calculated for C₂₀H₂₁ClN₃O₂(M+H)⁺: m/z=370.1; found 370.1.

### Step 6. Methyl ((1R,3R)-3-(6-((1-((1S,2S)-1-carbamoyl-2-methylcyclopropyl)-3,4-dimethyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 54, Step 8 and 11, with benzyl (1S,2S)-1-(6-chloro-3,4-dimethyl-1H-pyrazolo[3,4-b]pyridin-1-yl)-2-methylcyclopropane-1-carboxylate replacing benzyl (1S,2S)-1-(3-(bis(tert-butoxycarbonyl)amino)-6-bromo-1H-pyrazolo[3,4-b]pyridin-1-yl)-2-methylcyclopropane-1-carboxylate. LCMS calculated for C₂₇H₃₀D₄N₉O₄ (M+H)⁺: m/z=552.3; found 552.3. ¹H NMR (600 MHz, DMSO) δ 11.20 (s, 1H), 8.22 (s, 1H), 7.66 (s, 1H), 7.38 (s, 1H), 7.33 (s, 1H), 6.80 (s, 1H), 6.72 (s, 1H), 4.97-4.91 (m, 1H), 3.54 (s, 3H), 2.63 (s, 3H), 2.57 (s, 3H), 2.35 (dd, J=13.7, 7.7 Hz, 1H), 2.23-2.19 (m, 1H), 2.17-2.11 (m, 1H), 2.07-2.02 (m, 1H), 1.95 (dd, J=13.7, 9.5 Hz, 1H), 1.77 (dd, J=7.9, 5.0 Hz, 1H), 1.67-1.63 (m, 1H), 1.37 (dd, J=9.7, 4.9 Hz, 1H), 1.32 (d, J=6.3 Hz, 3H).

### Example 57. Methyl ((1R,3R)-3-(6-((1-((1S,2S)-1-carbamoyl-2-methylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate

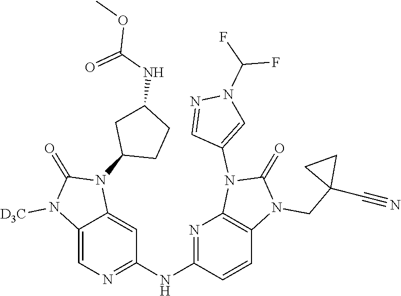 [SMILES: [2H]C([2H])([2H])n1c(=O)n([C@@H]2CC[C@@H](NC(=O)OC)C2)c2cc(Nc3ccc4c(n3)n(-c3cnn(C(F)F)c3)c(=O)n4CC3(C#N)CC3)ncc21]

The title compound was prepared according to the procedures described in Example 54, Step 8-11, with methyl ((1R,3R)-3-(6-amino-3-(methyl-d₃)-2-oxo-2,3-dihydro-1H-imidazo[4,5-c]pyridin-1-yl)cyclopentyl)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 8 and methyl-d₃-zinc chloride replacing methylzinc chloride in Step 10. LCMS calculated for: C₂₆H₂₆D₆N₉O₄(M+H)⁺: m/z=540.3; found 540.3.

### Example 58. Methyl ((1R,3R)-3-(6-((1-((1S,2S)-1-carbamoyl-2-(difluoromethyl)cyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

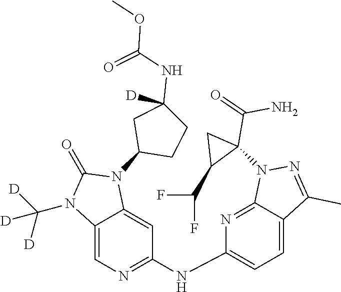 [SMILES: [2H]C([2H])([2H])n1c(=O)n([C@@H]2CC[C@@]([2H])(NC(=O)OC)C2)c2cc(Nc3ccc4c(C)nn([C@@]5(C(N)=O)C[C@@H]5C(F)F)c4n3)ncc21]

The title compound was prepared according to the procedures described in Example 54 with methyl (1S,2S)-1-amino-2-(difluoromethyl)cyclopropane-1-carboxylate (Example 42, Step 2) replacing benzyl (1S,2S)-1-amino-2-methylcyclopropane-1-carboxylate in step 5. LCMS calculated for C₂₆H₂₆D₄F₂N₉O₄ (M+H)⁺: m/z=574.3; found 574.3. ¹H NMR (500 MHz, DMSO) δ 11.35 (s, 1H), 8.26 (d, J=8.6 Hz, 1H), 8.23 (s, 1H), 7.63 (s, 1H), 7.39 (s, 1H), 7.34 (s, 1H), 7.03 (s, 1H), 6.98 (d, J=8.6 Hz, 1H), 6.46-6.14 (m, 1H), 4.94 (q, J=8.5 Hz, 1H), 3.56 (s, 3H), 2.52 (m, 1H), 2.49 (s, 3H), 2.32 (dd, J=13.8, 7.5 Hz, 1H), 2.28 (d, J=6.8 Hz, 1H), 2.26-2.13 (m, 2H), 2.11-1.91 (m, 3H), 1.66 (m, 1H).

### Example 59. Methyl ((1R,3R)-3-(6-((7-((1R,2S)-1-carbamoyl-2-methylcyclopropyl)-5-(methyl-d₃)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

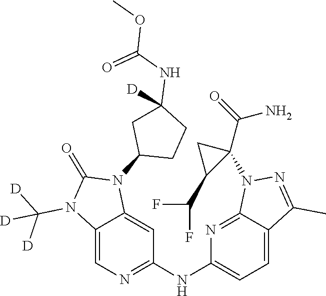 [SMILES: [2H]C([2H])([2H])n1c(=O)n([C@@H]2CC[C@@]([2H])(NC(=O)OC)C2)c2cc(Nc3ccc4c(C)nn([C@@]5(C(N)=O)C[C@@H]5C(F)F)c4n3)ncc21]

### Step 1. Benzyl (1R,2S)-1-(2-chloroimidazo[1,5-b]pyridazin-7-yl)-2-methylcyclopropane-1-carboxylate

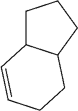

The title compound was prepared according to the procedures described in Example 5, Step 1-2, with (1R,2S)-1-((benzyloxy)carbonyl)-2-methylcyclopropane-1-carboxylic acid, replacing 3-fluoro-1-(isopropoxycarbonyl)cyclobutane-1-carboxylic acid in Step 1. The crude product was purified by flash column chromatography on silica (0-40% EtOAc in hexanes) to afford the title compound. LCMS calculated for C₁₈H₁₇ClN₃O₂(M+H)⁺: m/z=342.1; found 342.1.

### Step 2. Benzyl (1R,2S)-1-(2-chloro-5-iodoimidazo[1,5-b]pyridazin-7-yl)-2-methylcyclopropane-1-carboxylate

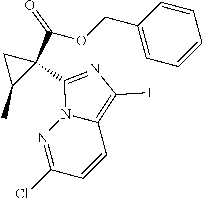 [SMILES: C[C@H]1C[C@]1(C(=O)OCc1ccccc1)c1nc(I)c2ccc(Cl)nn12]

The title compound was prepared according to the procedures described in Example 6, Step 1, with benzyl (1R,2S)-1-(2-chloroimidazo[1,5-b]pyridazin-7-yl)-2-methylcyclopropane-1-carboxylate, replacing isopropyl 1-(2-chloroimidazo[1,5-b]pyridazin-7-yl)-3-fluorocyclobutane-1-carboxylate. The crude product was purified by flash column chromatography on silica (0-40% EtOAc in hexanes) to afford the title compound (2.72 g, 92% yield). LCMS calculated for C₁₈H₁₆ClN₃O₂(M+H)⁺: m/z=468.0; found 468.1.

### Step 3. Benzyl (1R,2S)-1-(2-chloro-5-(methyl-d₃)imidazo[1,5-b]pyridazin-7-yl)-2-methylcyclopropane-1-carboxylate

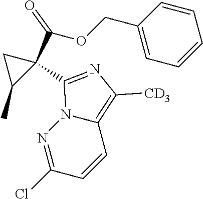 [SMILES: [2H]C([2H])([2H])c1nc([C@@]2(C(=O)OCc3ccccc3)C[C@@H]2C)n2nc(Cl)ccc12]

The title compound was prepared according to the procedures described in Example 54, Step 10, with benzyl (1R,2S)-1-(2-chloroimidazo[1,5-b]pyridazin-7-yl)-2-methylcyclopropane-1-carboxylate and (methyl-d₃)zinc(II) chloride, replacing benzyl (1S,2S)-1-(3-iodo-6-((1-((1R,3R)-3-((methoxycarbonyl)amino)-cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate and methylzinc chloride, respectively. The crude material was purified by flash column chromatography on silica (0-60% EtOAc in hexanes) to afford the title compound (46.7 mg, 23% yield). LCMS calculated for C₁₉H₁₆D₃ClN₃O₂(M+H)⁺: m/z=359.1; found 359.2.

### Step 4. Methyl ((1R,3R)-3-(6-((7-((1R,2S)-1-carbamoyl-2-methylcyclopropyl)-5-(methyl-d₃)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 5, Step 3-5, replacing isopropyl 1-(2-chloroimidazo[1,5-b]pyridazin-7-yl)-3-fluorocyclobutane-1-carboxylate with benzyl (1R,2S)-1-(2-chloro-5-(methyl-d₃)imidazo[1,5-b]pyridazin-7-yl)-2-methylcyclopropane-1-carboxylate in step 3, lithium hydroxide with sodium hydroxide in step 4, and 0.4 M ammonia in dioxane with ammonium chloride in step 5. The final crude mixture was purified by flash column chromatography on silica (0-40% MeOH in CH₂Cl₂) then further purified using prep-LCMS (XBridge C18 column, eluting with a gradient of acetonitrile/water containing 0.1% NH₄OH, at flow rate of 60 mL/min) to give the title compound. LCMS calculated for: C₂₆H₂₅D₇N₉O₄(M+H)⁺: m/z=541.3; found 541.3. ¹H NMR (500 MHz, DMSO-d₆) δ 9.78 (s, 1H), 8.16 (s, 1H), 8.07 (s, 1H), 7.84 (d, J=9.5 Hz, 1H), 7.20 (s, 1H), 7.11 (s, 1H), 6.69 (s, 1H), 6.60 (d, J=9.5 Hz, 1H), 5.21-5.10 (m, 1H), 3.50 (s, 3H), 2.49-2.42 (m, 1H), 2.26-2.16 (m, 1H), 2.15-2.09 (m, 1H), 2.08-1.92 (m, 3H), 1.68-1.58 (m, 1H), 1.57-1.46 (m, 1H), 1.27 (d, J=6.2 Hz, 3H), 1.05-0.99 (m, 1H).

### Example 60. Methyl ((1R,3R)-3-(6-((7-((1R,2S)-1-carbamoyl-2-methylcyclopropyl)-5-(trifluoromethyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

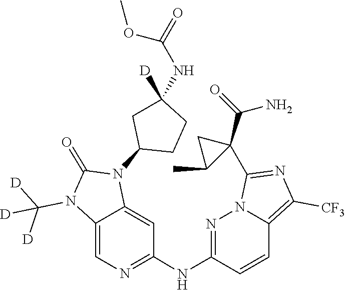 [SMILES: [2H]C([2H])([2H])n1c(=O)n([C@@H]2CC[C@@]([2H])(NC(=O)OC)C2)c2cc(Nc3ccc4c(C(F)(F)F)nc([C@@]5(C(N)=O)C[C@@H]5C)n4n3)ncc21]

### Step 1. Benzyl (1R,2S)-1-(2-chloro-5-(trifluoromethyl)imidazo[1,5-b]pyridazin-7-yl)-2-methylcyclopropane-1-carboxylate

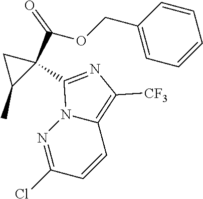 [SMILES: C[C@H]1C[C@]1(C(=O)OCc1ccccc1)c1nc(C(F)(F)F)c2ccc(Cl)nn12]

To a solution of benzyl (1R,2S)-1-(2-chloro-5-iodoimidazo[1,5-b]pyridazin-7-yl)-2-methylcyclopropane-1-carboxylate (Example 59—Step 2) (495 mg, 1.06 mmol) in DMF (5.3 mL) was added copper(I) iodide (1.0 g, 5.29 mmol), HMPA (0.92 mL, 5.29 mmol), and methyl 2,2-difluoro-2-(fluorosulfonyl)acetate (0.94 mL, 7.41 mmol). The mixture was heated to 100° C. under nitrogen for 2 h. The crude reaction was cooled down to room temperature and quenched with water. The mixture was extracted with Et₂O. The combined organic extracts were washed with saturated NaHCO₃ solution, then with brine, dried over Na₂SO4, filtered, and concentrated under reduced pressure. The residue was purified by flash chromatography (0-40% EtOAc in hexanes) to afford the title compound (416 mg, 96% yield). LCMS calculated for: C₁₉H₁₆ClF₃N₃O₂ (M+H)⁺: m/z=410.1; found 410.1.

### Step 2. Methyl ((1R,3R)-3-(6-((7-((1R,2S)-1-carbamoyl-2-methylcyclopropyl)-5-(trifluoromethyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 5, Step 3-5, replacing isopropyl 1-(2-chloroimidazo[1,5-b]pyridazin-7-yl)-3-fluorocyclobutane-1-carboxylate with benzyl (1R,2S)-1-(2-chloro-5-(trifluoromethyl)imidazo[1,5-b]pyridazin-7-yl)-2-methylcyclopropane-1-carboxylate in step 3, lithium hydroxide with sodium hydroxide in step 4, and 0.4 M ammonia in dioxane with ammonium chloride in step 5. The final crude mixture was purified by flash column chromatography on silica (0-40% MeOH in CH₂Cl₂) then further purified using prep-LCMS (XBridge C18 column, eluting with a gradient of acetonitrile/water containing 0.1% NH₄OH, at flow rate of 60 mL/min) to give the title compound. LCMS calculated for: C₂₆H₂₅D₄F₃N₉O₄ (M+H)⁺: m/z=592.3; found 592.3. ¹H NMR (600 MHz, DMSO-d₆) δ 10.16 (s, 1H), 8.13 (s, 1H), 8.11 (s, 1H), 7.99 (d, J=9.9 Hz, 1H), 7.21 (s, 1H), 7.19 (s, 1H), 7.07 (d, J=9.9 Hz, 1H), 7.01 (s, 1H), 5.22-5.01 (m, 1H), 3.51 (s, 3H), 2.50-2.44 (m, 1H), 2.23-2.16 (m, 1H), 2.16-2.10 (m, 1H), 2.10-1.97 (m, 3H), 1.69-1.60 (m, 1H), 1.60-1.53 (m, 1H), 1.31 (d, J=6.3 Hz, 3H), 1.16-1.11 (m, 1H). ¹⁹F NMR (565 MHz, DMSO-d₆) δ −58.00 (s, 3F).

### Example 61. Methyl ((1S,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

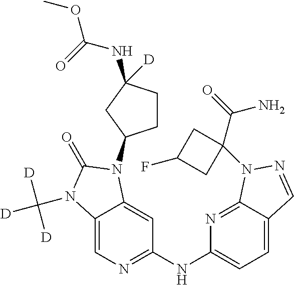 [SMILES: [2H]C([2H])([2H])n1c(=O)n([C@@H]2CC[C@]([2H])(NC(=O)OC)C2)c2cc(Nc3ccc4cnn(C5(C(N)=O)CC(F)C5)c4n3)ncc21]

### Step 1. Methyl ((1S,3R)-3-(6-amino-3-(methyl-d₃)-2-oxo-2,3-dihydro-1H-imidazo[4,5-c]pyridin-1-yl)cyclopentyl-1-d)carbamate

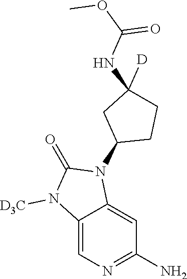 [SMILES: [2H]C([2H])([2H])n1c(=O)n([C@@H]2CC[C@]([2H])(NC(=O)OC)C2)c2cc(N)ncc21]

The title compound was prepared according to the procedures described in Intermediate A, Step 1 to 9, with the later peak replacing the earlier peak from CombiFlash in Step 1. LCMS calculated for C₁₄H₁₆D₄N₅O₃(M+H)⁺: m/z=310.2; found: 310.2.

### Step 2. Methyl ((1S,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 24, Step 1 to 8, with methyl ((1S,3R)-3-(6-amino-3-(methyl-d₃)-2-oxo-2,3-dihydro-1H-imidazo[4,5-c]pyridin-1-yl)cyclopentyl-1-d)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 7. LCMS calculated for C₂₅H₂₅D₄FN₉O₄(M+H)⁺: m/z=542.3; found 542.3. ¹H NMR (500 MHz, DMSO) δ 11.18 (brs, 1H), 8.25 (s, 1H), 8.20 (d, J=8.6 Hz, 1H), 8.16 (s, 1H), 7.78 (s, 1H), 7.50 (s, 1H), 7.40 (s, 1H), 7.04-6.97 (m, 2H), 5.37-5.13 (m, 1H), 4.90 (p, J=8.7 Hz, 1H), 3.55 (s, 3H), 3.47-3.39 (m, 2H), 3.13-2.99 (m, 2H), 2.30 (dd, J=12.5, 8.3 Hz, 1H), 2.25-2.16 (m, 1H), 2.15-1.96 (m, 3H), 1.82 (ddd, J=12.4, 9.6, 6.2 Hz, 1H).

### Example 62. Methyl (3-(6-((1-((1S,2S)-1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate

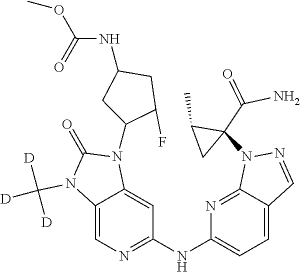 [SMILES: [2H]C([2H])([2H])n1c(=O)n(C2CC(NC(=O)OC)CC2F)c2cc(Nc3ccc4cnn([C@@]5(C(N)=O)C[C@@H]5C)c4n3)ncc21]

### Step 1. Benzyl cyclopent-3-en-1-ylcarbamate

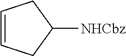 [SMILES: O=C(NC1CC=CC1)OCc1ccccc1]

To a flask containing cyclopent-3-en-1-amine hydrochloride (15.0 g, 125 mmol) and sodium bicarbonate (31.6 g, 376 mmol) in THF (300 mL) and water (50 mL) at 0° C. was added benzyl carbonochloridate (22 mL, 157 mmol) dropwise via addition funnel. After the addition of benzyl carbonochloridate, the reaction mixture was allowed to warm up to r.t. and stirred at r.t. for 16 h. The reaction mixture was treated with sat. NaHCO₃ solution (100 mL), followed by extraction with EtOAc (200 mL) for 3 times. The organic phase was combined and washed with brine, dried over MgSO₄, and concentrated in vacuo. The crude material was used in the next step without further purification. LCMS calculated for C₁₃H₁₆NO₂ (M+H)⁺: m/z=218.1; found: 218.1.

### Step 2. Benzyl ((1R,3r,5S)-6-oxabicyclo[3.1.0]hexan-3-yl)carbamate

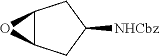 [SMILES: O=C(N[C@@H]1C[C@@H]2O[C@@H]2C1)OCc1ccccc1]

To a flask containing benzyl cyclopent-3-en-1-ylcarbamate (25.0 g, 115 mmol) and sodium bicarbonate (9.7 g, 115 mmol) in CH₂Cl₂ (300 mL) at 0° C. was added m-CPBA (33.5 g, 150 mmol) in portions. The reaction was allowed to warm up to r.t. and stirred at r.t. for 6 h. The reaction mixture was quenched with sat. Na₂S₂O₃ solution (50 mL) at 0° C., followed by extraction with CH₂Cl₂ (200 mL) for 3 times. The organic phase was combined and washed with brine, dried over MgSO₄, and concentrated in vacuo. The crude material was purified by Biotage Isolera to give the desired product as colorless oil. LCMS calculated for C₁₃H₁₆NO₃ (M+H)⁺: m/z=234.1; found: 234.1.

### Step 3. Benzyl ((1R,3S,4S)-3-fluoro-4-hydroxycyclopentyl)carbamate and benzyl ((1S,3R,4R)-3-fluoro-4-hydroxycyclopentyl)carbamate

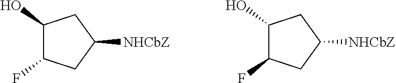

To a HDPE bottle containing benzyl ((1R,3r,5S)-6-oxabicyclo[3.1.0]hexan-3-yl)carbamate (25 g, 107 mmol) in DCM (120 mL) at 0° C. was added hydrogen fluoride pyridine (9.2 g, 322 mmol, 70% w/w). The reaction was allowed to warm up to r.t. and stirred at r.t. for 16 h. The reaction mixture was quenched with 3.0 M NaOH solution at 0° C. until pH>10, followed by extraction with CH₂Cl₂ (200 mL) for 3 times. The organic phase was combined and washed with brine, dried over MgSO₄, and concentrated in vacuo. The crude material was purified by Biotage Isolera to give the desired products as colorless oil. The racemic mixture was further separated by chiral SFC (Phenomenex-Amylose-1, 21 mm×250 mm, 5 μm column, eluting with 15% methanol in CO₂, at flow rate of 55 mL/min over 9 min). The first peak was collected at 6.0 min as a single enantiomer. The second peak was collected at 7.5 min as a single enantiomer. Both peaks were individually carried forward through next steps. Peak 1: LCMS calculated for C₁₃H₁₇FNO₃ (M+H)⁺: m/z=254.1; found: 254.1. Peak 2: LCMS calculated for C₁₃H₁₇FNO₃ (M+H)⁺: m/z=254.1; found: 254.1.

### Step 4. Benzyl (3-(6-chloro-3-(methyl-d₃)-2-oxo-2,3-dihydro-1H-imidazo[4,5-c]pyridin-1-yl)-4-fluorocyclopentyl)carbamate

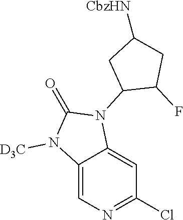 [SMILES: [2H]C([2H])([2H])n1c(=O)n(C2CC(NC(=O)OCc3ccccc3)CC2F)c2cc(Cl)ncc21]

The title compound was prepared according to the procedures described in Intermediate A, Step 2 to 8, with benzyl (3-fluoro-4-hydroxycyclopentyl)carbamate (single enantiomer, Peak 1 from Step 3) replacing tert-butyl ((1R,3S)-3-hydroxycyclopentyl-3-d)carbamate in Step 2. LCMS calculated for C₂₀H₁₈D₃ClFN₄O₃(M+H)⁺: m/z=422.1; found: 422.1.

### Step 5. 1-(4-Amino-2-fluorocyclopentyl)-6-chloro-3-(methyl-d₃)-1,3-dihydro-2H-imidazo[4,5-c]pyridin-2-one

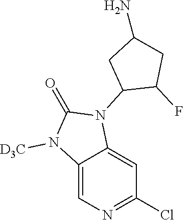 [SMILES: [2H]C([2H])([2H])n1c(=O)n(C2CC(N)CC2F)c2cc(Cl)ncc21]

To a vial containing benzyl (3-(6-chloro-3-(methyl-d₃)-2-oxo-2,3-dihydro-1H-imidazo[4,5-c]pyridin-1-yl)-4-fluorocyclopentyl)carbamate (800 mg, 1.9 mmol) was added TFA (3.0 mL). After stirring at 75° C. for 4 h, the reaction mixture was concentrated in vacuo. The crude material was dissolved in CH₂Cl₂ (20 mL) and the pH of the mixture was adjusted to ˜10 with ammonia aqueous solution and then extracted into CH₂Cl₂. The organic phase was washed with brine, dried over MgSO₄, and concentrated in vacuo. The obtained crude product was used in the next step without further purification. LCMS calculated for C₁₂H₁₂D₃ClFN₄O (M+H)⁺: m/z=288.1; found: 288.1.

### Step 6. Methyl (3-(6-amino-3-(methyl-d₃)-2-oxo-2,3-dihydro-1H-imidazo[4,5-c]pyridin-1-yl)-4-fluorocyclopentyl)carbamate

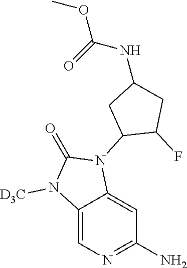 [SMILES: [2H]C([2H])([2H])n1c(=O)n(C2CC(NC(=O)OC)CC2F)c2cc(N)ncc21]

The title compound was prepared according to the procedures described in Alternative Synthesis of Intermediate A, Step 5 to 6, with 1-(4-amino-2-fluorocyclopentyl)-6-chloro-3-(methyl-d₃)-1,3-dihydro-2H-imidazo[4,5-c]pyridin-2-one replacing 1-((1R,3R)-3-aminocyclopentyl-3-d)-6-chloro-3-(methyl-d₃)-1,3-dihydro-2H-imidazo[4,5-c]pyridin-2-one in Step 5. LCMS calculated for C₁₄H₁₆D₃FN₅O₃(M+H)⁺: m/z=327.1; found: 327.1.

### Step 7. Benzyl (1S,2S)-1-(6-bromo-1H-pyrazolo[3,4-b]pyridin-1-yl)-2-methylcyclopropane-1-carboxylate

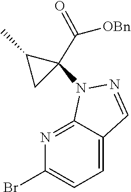 [SMILES: C[C@H]1C[C@]1(C(=O)OCc1ccccc1)n1ncc2ccc(Br)nc21]

The title compound was prepared according to the procedures described in Example 24, Step 6, with benzyl (1S,2S)-1-(3-amino-6-bromo-1H-pyrazolo[3,4-b]pyridin-1-yl)-2-methylcyclopropane-1-carboxylate (Example 54, Step 6) replacing isopropyl 1-(3-amino-6-bromo-1H-pyrazolo[3,4-b]pyridin-1-yl)-3-fluorocyclobutane-1-carboxylate in Step 6. LCMS calculated for C₁₈H₁₇BrN₃O₂(M+H)⁺: m/z=386.0/388.0; found 386.0/388.0.

### Step 8. Methyl (3-(6-((1-((1S,2S)-1-carbamoyl-2-methylcyclopropyl)-1H-pyrazolo[3,4-b]pyridin-6-yl)amino)-3-(methyl-d₃)-2-oxo-2,3-dihydro-H-imidazo[4,5-c]pyridin-1-yl)-4-fluorocyclopentyl)carbamate

The title compound was prepared according to the procedures described in Example 24, Step 7 to 8, with methyl (3-(6-amino-3-(methyl-d₃)-2-oxo-2,3-dihydro-1H-imidazo[4,5-c]pyridin-1-yl)-4-fluorocyclopentyl)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate and benzyl (1S,2S)-1-(6-bromo-1H-pyrazolo[3,4-b]pyridin-1-yl)-2-methylcyclopropane-1-carboxylate replacing isopropyl 1-(6-bromo-1H-pyrazolo[3,4-b]pyridin-1-yl)-3-fluorocyclobutane-1-carboxylate in Step 7. LCMS calculated for C₂₅H₂₆D₃FN₉O₄(M+H)⁺: m/z=541.3; found 541.3. ¹H NMR (500 MHz, DMSO) δ 11.22 (brs, 1H), 8.25 (s, 1H), 8.20 (d, J=8.6 Hz, 1H), 8.08 (s, 1H), 7.58 (s, 1H), 7.47 (d, J=7.4 Hz, 1H), 7.40 (s, 1H), 7.08 (d, J=8.6 Hz, 1H), 6.87 (s, 1H), 5.40-5.22 (m, 1H), 5.17-5.00 (m, 1H), 4.45 (q, J=7.3 Hz, 1H), 3.56 (s, 3H), 2.94-2.83 (m, 1H), 2.49-2.36 (m, 1H), 2.22-1.95 (m, 3H), 1.78 (dd, J=8.0, 5.1 Hz, 1H), 1.44 (dd, J=9.7, 5.1 Hz, 1H), 1.35 (d, J=6.3 Hz, 3H).

### Example 63. Methyl (3-(6-((1-((1S,2S)-1-carbamoyl-2-ethylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate

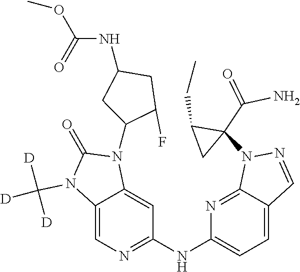 [SMILES: [2H]C([2H])([2H])n1c(=O)n(C2CC(NC(=O)OC)CC2F)c2cc(Nc3ccc4cnn([C@@]5(C(N)=O)C[C@@H]5CC)c4n3)ncc21]

The title compound was prepared according to the procedures described in Example 43, Step 1 to 8, with methyl (3-(6-amino-3-(methyl-d₃)-2-oxo-2,3-dihydro-1H-imidazo[4,5-c]pyridin-1-yl)-4-fluorocyclopentyl)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate. LCMS calculated for C₂₆H₂₈D₃FN₉O₄(M+H)⁺: m/z=555.3; found 555.3.

### Example 64. Methyl (3-(6-((1-((1S,2S)-1-carbamoyl-2-methylcyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate

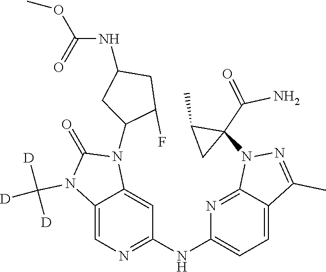 [SMILES: [2H]C([2H])([2H])n1c(=O)n(C2CC(NC(=O)OC)CC2F)c2cc(Nc3ccc4c(C)nn([C@@]5(C(N)=O)C[C@@H]5C)c4n3)ncc21]

The title compound was prepared according to the procedures described in Example 54, Step 1 to 11, with methyl (3-(6-amino-3-(methyl-d₃)-2-oxo-2,3-dihydro-1H-imidazo[4,5-c]pyridin-1-yl)-4-fluorocyclopentyl)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 8. LCMS calculated for C₂₆H₂₈D₃FN₉O₄(M+H)⁺: m/z=555.3; found 555.3. ¹H NMR (500 MHz, DMSO) δ 11.33 (s, 1H), 8.27 (s, 1H), 8.20 (d, J=8.6 Hz, 1H), 7.57-7.42 (m, 2H), 7.37 (s, 1H), 7.01 (d, J=8.6 Hz, 1H), 6.86 (s, 1H), 5.40-5.22 (m, 1H), 5.16-5.02 (m, 1H), 4.45 (q, J=7.3 Hz, 1H), 3.57 (s, 3H), 2.90-2.78 (m, 1H), 2.50-2.36 (m, 4H), 2.20-1.95 (m, 3H), 1.78 (dd, J=7.9, 5.0 Hz, 1H), 1.43 (dd, J=9.8, 5.0 Hz, 1H), 1.33 (d, J=6.3 Hz, 3H).

### Example 65. Methyl ((1R,3R)-3-(6-((1-((1S,2S)-1-carbamoyl-2-methylcyclopropyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl cyclopentyl-1-d)carbamate

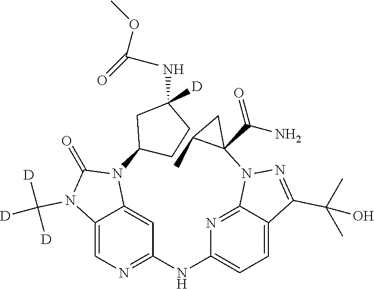

### Step 1. Benzyl (1S,2S)-1-(6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-3-(prop-1-en-2-yl)-1H-pyrazolo[3,4-b]pyridin-1-yl)-2-methylcyclopropane-1-carboxylate

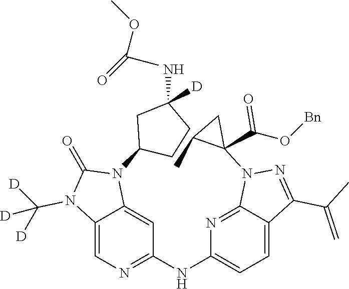

To a flask containing benzyl (1S,2S)-1-(3-iodo-6-((1-((1R,3R)-3-((methoxycarbonyl)amino)-cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (500 mg, 0.675 mmol, Example 54, step 9), [1,1′-bis(diphenylphosphino)ferrocene]-dichloropalladium(II), complex with dichloromethane (110 mg, 0.135 mmol), cesium carbonate (440 mg, 1.35 mmol), and isopropenylboronic acid pinacol ester (170 mg, 1.013 mmol) was evacuated and backfilled with nitrogen three times, followed by the addition of dioxane (4 mL) and water (0.8 mL). The vial was sealed and heated to 80° C. for 1 h. After cooling to r.t., the mixture was quenched with water and filtered, followed by extraction with EtOAc 3 times. The organic phases were combined and washed with brine, dried over MgSO₄ and concentrated in vacuo. The crude material was purified by Biotage Isolera to give the desired product. LCMS calculated for C₃₅H₃₅D₄N₈O₅ (M+H)⁺: m/z=655.3; found 655.3.

### Step 2. Benzyl (1S,2S)-1-(3-(2-hydroxypropan-2-yl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

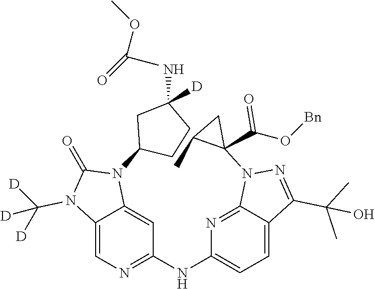

To a vial containing benzyl (1S,2S)-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(prop-1-en-2-yl)-1H-pyrazolo[3,4-b]pyridin-1-yl)-2-methylcyclopropane-1-carboxylate (442 mg, 0.675 mmol), iron(III) acetylacetonate (24 mg, 0.068 mmol), methyl 4-nitrobenzenesulfonate (220 mg, 1.01 mmol), and sodium bicarbonate (170 mg, 2.025 mmol) was added methanol (4 mL). The vial was cooled to 0° C. and phenylsilane (0.1 mL, 0.81 mmol) was added dropwise. The reaction mixture was allowed to warm up to r.t. and stirred at r.t. for 16 h before concentrated in vacuo. The crude material was purified by Biotage Isolera to give the desired product. LCMS calculated for C₃₅H₃₇D₄N₈O₆(M+H)⁺: m/z=673.3; found 673.3.

### Step 3. Methyl ((1R,3R)-3-(6-((1-((1S,2S)-1-carbamoyl-2-methylcyclopropyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 54, Step 11, with benzyl (1S,2S)-1-(3-(2-hydroxypropan-2-yl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate replacing benzyl (1S,2S)-1-(6-((1-((1R,3R)-3-((methoxycarbonyl)amino)-cyclopentyl-3-d)-3-(methyl-d₃)-2-oxo-2,3-dihydro-1H-imidazo[4,5-c]pyridin-6-yl)amino)-3-methyl-1H-pyrazolo[3,4-b]pyridin-1-yl)-2-methylcyclopropane-1-carboxylate. LCMS calculated for C₂₈H₃₂D₄N₉O₅(M+H)⁺: m/z=582.3; found 582.3. ¹H NMR (500 MHz, DMSO) δ 9.93 (s, 1H), 8.42 (s, 1H), 8.11 (d, J=8.8 Hz, 1H), 8.06 (s, 1H), 7.38 (s, 1H), 7.23 (s, 1H), 7.06 (d, J=8.8 Hz, 1H), 6.18 (s, 1H), 5.18 (s, 1H), 4.93-4.86 (m, 1H), 3.52 (s, 3H), 2.44 (dd, J=13.4, 7.4 Hz, 1H), 2.25-2.19 (m, 1H), 2.18-1.97 (m, 3H), 1.93 (dd, J=13.3, 9.4 Hz, 1H), 1.73 (dd, J=8.1, 4.8 Hz, 1H), 1.68-1.62 (m, 1H), 1.58 (s, 6H), 1.50 (dd, J=9.6, 4.7 Hz, 1H), 1.33 (d, J=6.3 Hz, 3H).

### Example 66. Methyl-d₃ ((1R,3R)-3-(6-((1-((1S,2S)-1-carbamoyl-2-methylcyclopropyl)-3-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

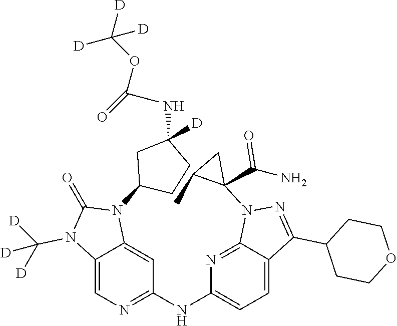

To a vial containing methyl ((1R,3R)-3-(6-((1-((1S,2S)-1-carbamoyl-2-methylcyclopropyl)-3-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate (20.0 mg, 0.033 mmol, Example 67) in MeOH-d₄ (1 mL; D %≥99.8%) was added Cs₂CO₃ (22.0 mg, 0.066 mmol). After heating at 120° C. for 2 h in microwave, the reaction mixture was cooled down, acidified with TFA and directly purified by prep-LCMS (XBridge C18 column, acetonitrile/water containing 0.1% TFA, at flow rate of 60 mL/min). LCMS calculated for: C₃₀H₃₁D₇N₉O₅(M+H)⁺: m/z=611.3; found: 611.3. ¹H NMR (500 MHz, DMSO) δ 8.22 (m, 2H), 7.78 (s, 1H), 7.37 (s, 1H), 7.32 (s, 1H), 6.98 (m, 1H), 6.75 (s, 2H), 4.96-4.91 (m, 1H), 3.95 (m, 2H), 3.54 (s, 3H), 3.45 (m, 1H), 3.25 (m, 1H), 2.36 (m, 1H), 2.21 (m, 1H), 2.17-2.11 (m, 2H), 2.09-2.03 (m, 1H), 1.95 (m 3H), 1.77 (m, 1H), 1.68-1.63 (m, 1H), 1.42 (m, 1H), 1.33 (d, J=6.3 Hz, 3H).

### Example 67. Methyl ((1R,3R)-3-(6-((1-((1S,2S)-1-carbamoyl-2-methylcyclopropyl)-3-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

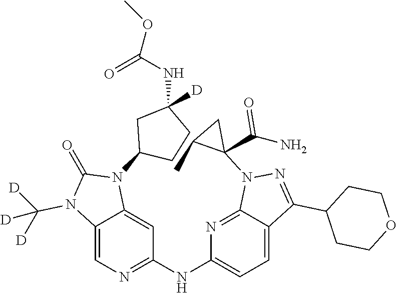 [SMILES: [2H]C([2H])([2H])n1c(=O)n([C@@H]2CC[C@@]([2H])(NC(=O)OC)C2)c2cc(Nc3ccc4c(C5CCOCC5)nn([C@@]5(C(N)=O)C[C@@H]5C)c4n3)ncc21]

### Step 1. Benzyl (1S,2S)-1-(3-(3,6-dihydro-2H-pyran-4-yl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

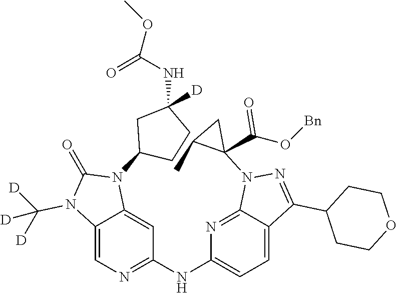 [SMILES: [2H]C([2H])([2H])n1c(=O)n([C@@H]2CC[C@@]([2H])(NC(=O)OC)C2)c2cc(Nc3ccc4c(C5CCOCC5)nn([C@@]5(C(=O)OCc6ccccc6)C[C@@H]5C)c4n3)ncc21]

To a vial containing benzyl (1S,2S)-1-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (750 mg, 1.013 mmol, Example 54, step 9) was added 2-(3,6-dihydro-2H-pyran-4-yl)-4,4,5,5-tetramethyl-1,3,2-dioxaborolane (298 mg, 1.418 mmol), tetrakis(triphenylphosphine)palladium(0) (234 mg, 0.203 mmol), and cesium carbonate (990 mg, 3.04 mmol), then evacuated and backfilled with N₂ three times. Dioxane (10 mL) and water (2.5 mL) were added by syringe and evacuated and backfilled with N₂, then heated to 75° C. for 2 h. The reaction mixture was cooled to r.t, then diluted with DCM, filtered over celite, and washed with DCM. The combining fractions were concentrated in vacuo and purified by Biotage Isolera to give the desired product. LCMS calculated for C₃₇H₃₇D₄N₈O₆ (M+H)⁺: m/z=697.3; found 697.3.

### Step 2. (1S,2S)-1-(6-((1-((1R,3R)-3-((Methoxycarbonyl)amino)cyclopentyl-3-d)-3-(methyl-d₃)-2-oxo-2,3-dihydro-1H-imidazo[4,5-c]pyridin-6-yl)amino)-3-(tetrahydro-2H-pyran-4-yl)-1H-pyrazolo[3,4-b]pyridin-1-yl)-2-methylcyclopropane-1-carboxylic acid

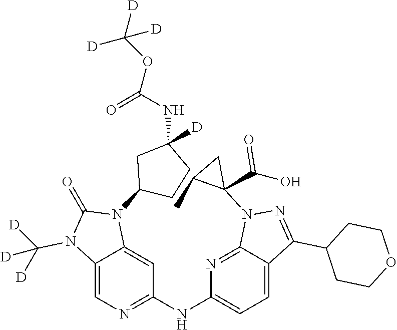 [SMILES: [2H]C([2H])([2H])OC(=O)N[C@]1([2H])CC[C@@H](n2c(=O)n(C([2H])([2H])[2H])c3cnc(Nc4ccc5c(C6CCOCC6)nn([C@@]6(C(=O)O)C[C@@H]6C)c5n4)cc32)C1]

To a vial containing benzyl (1S,2S)-1-(3-(3,6-dihydro-2H-pyran-4-yl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (400 mg, 0.57 mmol) in methanol (20 mL) was added Pd—C (60 mg, 0.056 mmol, 10% wet). The vial was evacuated and backfilled with H₂ three times, then heated to 50° C. overnight with H₂ balloon connected. The reaction mixture was cooled to r.t, then diluted with methanol, filtered through celite, and concentrated in vacuo to give the desired product. LCMS calculated for C₃₀H₃₃D₄N₈O₆(M+H)⁺: m/z=609.3; found 609.3.

### Step 3. Methyl ((1R,3R)-3-(6-((1-((1S,2S)-1-carbamoyl-2-methylcyclopropyl)-3-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The compound (1S,2S)-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(tetrahydro-2H-pyran-4-yl)-1H-pyrazolo[3,4-b]pyridin-1-yl)-2-methylcyclopropane-1-carboxylic acid (350 mg, 0.52 mmol, step 2) was added to a reaction vial containing HATU (310 mg, 0.8 mmol) and DIPEA (0.14 mL, 0.8 mmol) in DMF (2 mL) and THF (6 mL). The reaction vial was cooled to 0° C. before ammonia (2.5 mL, 1.0 mmol, 0.4 M in dioxane) was added, and it was then sealed and stirred at 50° C. for 2 h. Upon completion, the mixture was diluted with EtOAc and washed with 1 N NaOH aq. solution. The organic phase was dried over MgSO₄ and concentrated in vacuo. The reaction residue was diluted with MeCN and purified by prep-LCMS (XBridge C18 column, acetonitrile/water containing 0.1% TFA, at flow rate of 60 mL/min). LCMS calculated for: C₃₀H₃₄D₄N₉O₅(M+H)⁺: m/z=608.3; found: 608.3. ¹H NMR (500 MHz, DMSO) δ 10.90 (s, 1H), 8.30 (d, J=8.6 Hz, 1H), 8.22 (s, 1H), 7.78 (s, 1H), 7.37 (s, 1H), 7.32 (s, 1H), 6.98 (d, J=8.6 Hz, 1H), 6.75 (s, 1H), 4.96-4.91 (m, 1H), 3.95 (m, 2H), 3.54 (s, 3H), 3.45 (m, 1H), 3.25 (m, 1H), 2.46 (s, 3H), 2.36 (dd, J=13.6, 7.6 Hz, 1H), 2.21 (m, 1H), 2.17-2.11 (m, 2H), 2.09-2.03 (m, 1H), 1.95 (m 3H), 1.77 (dd, J=8.0, 4.9 Hz, 1H), 1.68-1.63 (m, 1H), 1.42 (dd, J=9.7, 4.9 Hz, 1H), 1.33 (d, J=6.3 Hz, 3H).

### Example 68. Methyl-d3 ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate

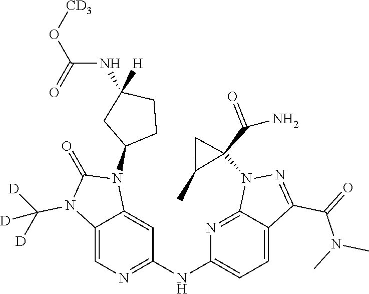

### Step 1. Benzyl (1S,2S)-1-(6-chloro-3-(dimethylcarbamoyl)-1H-pyrazolo[3,4-b]pyridin-1-yl)-2-methylcyclopropane-1-carboxylate

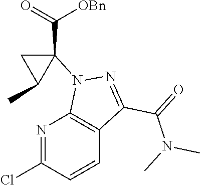

To a solution of benzyl (1S,2S)-1-(6-chloro-3-iodo-1H-pyrazolo[3,4-b]pyridin-1-yl)-2-methylcyclopropane-1-carboxylate (3.0 g, 6.4 mmol) in DMF (60 mL) were added [1,1′-bis(diphenylphosphino)ferrocene]dichloropalladium(II), complex with dichloromethane (1.0 g, 1.2 mmol), triethylamine (2.7 mL, 19.2 mmol), and dimethylamine (6.4 mL, 12.8 mmol). The reaction mixture was heated to 70° C. for 4 h under CO gas. After cooling to r.t., the mixture was filtered and concentrated in vacuo. The crude material was purified by Biotage Isolera to give the desired product. LCMS calculated for C₂₁H₂₂ClN₄O₃(M+H)⁺: m/z=413.1; found 413.2.

### Step 2. Benzyl (1S,2S)-1-(3-(dimethylcarbamoyl)-6-((1-((1R,3R)-3-(((methoxy-d3)carbonyl)amino)cyclopentyl)-3-(methyl-d3)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate

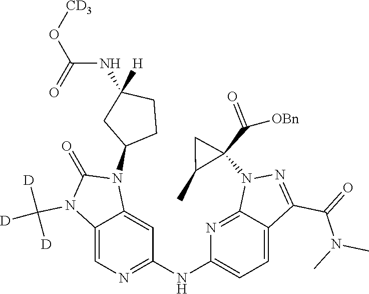 [SMILES: [2H]C([2H])([2H])OC(=O)N[C@@H]1CC[C@@H](n2c(=O)n(C([2H])([2H])[2H])c3cnc(Nc4ccc5c(C(=O)N(C)C)nn([C@@]6(C(=O)OCc7ccccc7)C[C@@H]6C)c5n4)cc32)C1]

A vial containing methyl-d3 ((1R,3R)-3-(6-amino-3-(methyl-d3)-2-oxo-2,3-dihydro-1H-imidazo[4,5-c]pyridin-1-yl)cyclopentyl)carbamate (0.23 g, 0.7 mmol), benzyl (1S,2S)-1-(6-chloro-3-(dimethylcarbamoyl)-1H-pyrazolo[3,4-b]pyridin-1-yl)-2-methylcyclopropane-1-carboxylate (0.3 g, 0.7 mmol), tris(dibenzylideneacetone)-dipalladium(0) (0.13 g, 0.14 mmol), 4,5-bis(diphenylphosphino)-9,9-dimethylxanthene (0.17 g, 0.3 mmol), and cesium carbonate (0.7 g, 2.1 mmol) was evacuated and backfilled with nitrogen three times, followed by the addition of 1,4-dioxane (7 mL). The vial was sealed and heated to 100° C. for 4 h. After cooling to r.t., the mixture was filtered and concentrated in vacuo. The crude material was purified by Biotage Isolera to give the desired product. LCMS calculated for C₃₅H₃₄D₆N₉O₆ (M+H)⁺: m/z=688.3; found 688.4.

### Step 3. Methyl-d3 ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate

This compound was prepared according to the procedures described in Example 33 in step 4 and final purification was done by prep-LCMS (XBridge C18 column, eluting with acetonitrile/aqueous 0.1% NH₄OH, at flow rate of 60 mL/min). LCMS calculated for C₂₈H₂₉D₆N₁₀O₅ (M+H)⁺: m/z=597.3; found 597.4. ¹H NMR (600 MHz, DMSO) δ 10.11 (s, 1H), 8.44 (s, 1H), 8.12-8.03 (m, 2H), 7.35 (s, 1H), 7.26 (d, J=7.3 Hz, 1H), 7.16 (d, J=8.8 Hz, 1H), 6.84 (d, J=4.2 Hz, 1H), 4.94-4.81 (m, 1H), 4.29-4.19 (m, 1H), 3.39 (s, 3H), 3.05 (s, 3H), 2.49-2.40 (m, 1H), 2.26-2.18 (m, 1H), 2.18-2.02 (m, 3H), 1.99-1.90 (m, 1H), 1.75 (dd, J=8.3, 4.8 Hz, 1H), 1.71-1.62 (m, 1H), 1.45 (dd, J=9.7, 4.7 Hz, 1H), 1.35 (d, J=6.3 Hz, 3H).

### Example 69-70

The compounds of Examples 69-70 in Table 2 were prepared according to the procedures described in Example 68.

TABLE 2

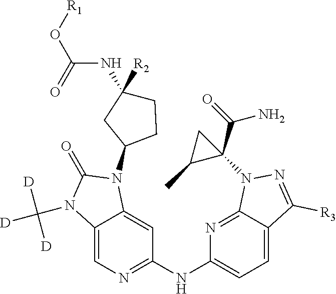

| Example | R₁; R₂; R₃ | LCMS | ¹H-NMR |
|---|---|---|---|
| 69 | R₁ = CD₃ | calculated for | ¹H NMR (500 MHz, DMSO) δ 10.11 (s, |
| | R₂ = D | C₂₈H₂₈D₇N₁₀O₅ | 1H), 8.44 (s, 1H), 8.10-8.03 (m, 2H), |
| | R₃ = NMe₂ | (M + H)⁺: m/z = | 7.36 (s, 1H), 7.25 (s, 1H), 7.16 (d, J = |
| | | 598.3; found | 8.8 Hz, 1H), 6.84 (s, 1H), 4.95-4.80 |
| | | 598.4 | (m, 1H), 3.39 (s, 3H), 3.05 (s, 3H), 2.45 |
| | | | (dd, J = 13.3, 7.5 Hz, 1H), 2.24-2.17 |
| | | | (m, 1H), 2.16-2.01 (m, 3H), 1.94 (dd, |
| | | | J = 13.3, 9.3 Hz, 1H), 1.75 (dd, J = 8.3, |
| | | | 4.8 Hz, 1H), 1.69-1.61 (m, 1H), 1.45 |
| | | | (dd, J = 9.8, 4.7 Hz, 1H), 1.35 (d, J = 6.3 |
| | | | Hz, 3H). |
| 70 | R₁ = Me | calculated for | ¹H NMR (500 MHz, DMSO) δ 10.10 (s, |
| | R₂ = D | C₂₈H₂₅D₁₀N₁₀O₅ | 1H), 8.44 (s, 1H), 8.10-8.04 (m, 2H), |
| | R₃ = N(CD₃)₂ | (M + H)⁺: m/z = | 7.35 (s, 1H), 7.25 (s, 1H), 7.16 (d, J = |
| | | 601.3; found | 8.8 Hz, 1H), 6.83 (s, 1H), 4.97-4.75 |
| | | 601.4 | (m, 1H), 3.51 (s, 3H), 2.45 (dd, J = 13.3, |
| | | | 7.5 Hz, 1H), 2.25-2.18 (m, 1H), 2.18- |
| | | | 2.02 (m, 3H), 1.94 (dd, J = 13.3, 9.3 Hz, |
| | | | 1H), 1.75 (dd, J = 8.3, 4.8 Hz, 1H), 1.70- |
| | | | 1.61 (m, 1H), 1.46 (dd, J = 9.8, 4.7 Hz, |
| | | | 1H), 1.35 (d, J = 6.3 Hz, 3H). |

### Example 71. Methyl ((1R,3R)-3-(6-((1-((1S,2S)-1-carbamoyl-2-methylcyclopropyl)-3-(4-fluoro-2-azabicyclo[2.1.1]hexan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

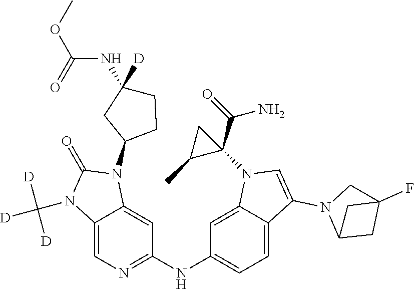 [SMILES: [2H]C([2H])([2H])n1c(=O)n([C@@H]2CC[C@@]([2H])(NC(=O)OC)C2)c2cc(Nc3ccc4c(N5CC6(F)CC5C6)cn([C@@]5(C(N)=O)C[C@@H]5C)c4c3)ncc21]

This compound was prepared according to the procedures described in Example 34 in steps 2-3 with 4-fluoro-2-azabicyclo[2.1.1]hexane hydrochloride replacing morpholine and final purification was done by prep-LCMS (XBridge C18 column, eluting with acetonitrile/aqueous 0.1% NH₄OH, at flow rate of 60 mL/min). LCMS calculated for C₃₀H₃₂D₄FN₁₀O₄(M+H)⁺: m/z=623.3; found 623.3. ¹H NMR (500 MHz, DMSO) δ 9.89 (s, 1H), 8.39 (s, 1H), 8.04 (s, 1H), 7.94 (d, J=8.8 Hz, 1H), 7.33-7.18 (m, 2H), 6.96 (d, J=8.8 Hz, 1H), 6.39 (s, 1H), 4.92-4.76 (m, 1H), 4.44-4.31 (m, 1H), 3.60-3.47 (m, 5H), 2.43 (dd, J=13.3, 7.5 Hz, 1H), 2.25-2.17 (m, 1H), 2.15-2.03 (m, 6H), 2.02-1.86 (m, 2H), 1.70-1.59 (m, 2H), 1.45 (dd, J=9.7, 4.6 Hz, 1H), 1.30 (d, J=6.3 Hz, 3H).

### Example 72. Methyl (1-((1S,3S)-1-carbamoyl-3-fluorocyclobutyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-3-yl)(methyl)carbamate

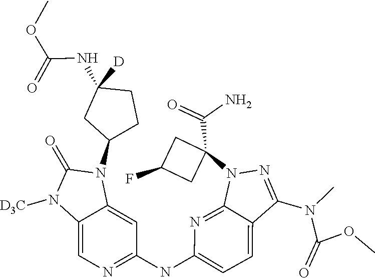 [SMILES: [2H]C([2H])([2H])n1c(=O)n([C@@H]2CC[C@@]([2H])(NC(=O)OC)C2)c2cc(Nc3ccc4c(N(C)C(=O)OC)nn([C@]5(C(N)=O)C[C@H](F)C5)c4n3)ncc21]

### Step 1. Isopropyl (1S,3S)-1-(6-chloro-3-iodo-1H-pyrazolo[3,4-b]pyridin-1-yl)-3-fluorocyclobutane-1-carboxylate

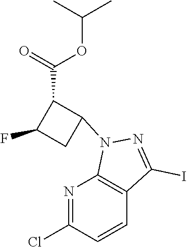 [SMILES: CC(C)OC(=O)[C@@H]1C(n2nc(I)c3ccc(Cl)nc32)C[C@H]1F]

This compound was prepared according to the procedures described in Example 24, steps 1-3 followed by procedure described in Example 34, step 1. The crude material was purified by Biotage Isolera to give the desired product. LCMS calculated for C₁₄H₁₅ClFN₃O₂ (M+H)⁺: m/z=437.9; found 437.9.

### Step 2. Methyl (1-((1S,3S)-1-carbamoyl-3-fluorocyclobutyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate

This compound was prepared according to the procedures described in Example 34 steps 2-3 with methyl methylcarbamate replacing morpholine. LCMS calculated for C₂₈H₃₀D₄FN₁₀O₆(M+H)⁺: m/z=629.3; found 629.4. ¹H NMR (600 MHz, DMSO) δ 10.01 (s, 1H), 8.16 (s, 1H), 8.06 (s, 1H), 7.91 (d, J=8.8 Hz, 1H), 7.40 (s, 1H), 7.24 (s, 1H), 7.02 (d, J=8.9 Hz, 1H), 6.63 (s, 1H), 5.40-5.21 (m, 1H), 5.09-5.00 (m, 1H), 3.73 (s, 3H), 3.54 (s, 3H), 3.39 (s, 3H), 3.37-3.27 (m, 2H), 3.05-2.92 (m, 2H), 2.42 (dd, J=13.3, 6.9 Hz, 1H), 2.22-2.06 (m, 3H), 2.00-1.92 (m, 1H), 1.70-1.62 (m, 1H).

### Example 73-74 and 107

The compounds of Examples 73, 74, and 107 of Table 3 were prepared according to the procedures described in Example 34 steps 2-3.

TABLE 3

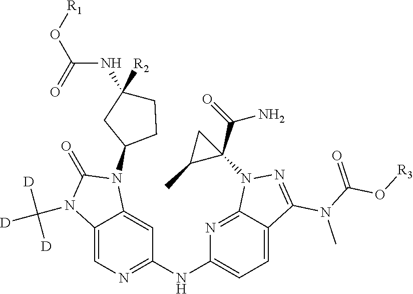

| Example | R₁; R₂; R₃ | LCMS | ¹H-NMR |
|---|---|---|---|
| 73 | R₁ = Me | calculated for | ¹H NMR (500 MHz, DMSO) δ 10.02 (s, 1H), |
| | R₂ = D | C₂₉H₃₃D₄N₁₀O₆ | 8.42 (s, 1H), 8.06 (s, 1H), 7.88 (d, J = 8.8 Hz, |
| | R₃ = Et | (M + H)⁺: m/z = | 1H), 7.34 (s, 1H), 7.23 (s, 1H), 7.06 (d, J = |
| | | 625.3; found | 8.9 Hz, 1H), 6.54 (s, 1H), 4.96-4.78 (m, |
| | | 625.4 | 1H), 4.23-4.11 (m, 2H), 3.51 (s, 3H), 3.37 |
| | | | (s, 3H), 2.44 (dd, J = 13.3, 7.5 Hz, 1H), 2.25- |
| | | | 2.17 (m, 1H), 2.17-2.00 (m, 3H), 1.93 (dd, |
| | | | J= 13.4, 9.3 Hz, 1H), 1.73 (dd, J = 8.2, 4.8 |
| | | | Hz, 1H), 1.69-1.60 (m, 1H), 1.48 (dd, J = |
| | | | 9.7, 4.7 Hz, 1H), 1.31 (d, J = 6.3 Hz, 3H), |
| | | | 1.20 (t, J = 7.1 Hz, 3H). |
| 74 | R₁ = Me | calculated for | ¹H NMR (500 MHz, DMSO) δ 10.02 (s, 1H), |
| | R₂ = D | C₂₈H₃₁D₄N₁₀O₆ | 8.42 (s, 1H), 8.06 (s, 1H), 7.87 (d, J = 8.8 Hz, |
| | R₃ = Me | (M + H)⁺: m/z = | 1H), 7.34 (s, 1H), 7.23 (s, 1H), 7.06 (d, J = |
| | | 611.3; found | 8.9 Hz, 1H), 6.57 (s, 1H), 4.96-4.81 (m, |
| | | 611.4 | 1H), 3.71 (s, 3H), 3.51 (s, 3H), 3.37 (s, 3H), |
| | | | 2.44 (dd, J = 13.4, 7.5 Hz, 1H), 2.25-2.17 |
| | | | (m, 1H), 2.17-2.00 (m, 3H), 1.93 (dd, J = |
| | | | 13.3, 9.3 Hz, 1H), 1.73 (dd, J = 8.2, 4.8 Hz, |
| | | | 1H), 1.69-1.60 (m, 1H), 1.47 (dd, J = 9.7, |
| | | | 4.7 Hz, 1H), 1.31 (d, J = 6.3 Hz, 3H). |
| 107 | R₁ = CD₃ | calculated for | |
| | R₂ = H | C₂₈H₂₉D₆N₁₀O₆ | |
| | R₃ = Me | (M + H)⁺: m/z = | |
| | | 613.3; found | |
| | | 613.4 | |

### Example 75. Methyl ((1R,3R)-3-(6-((1-((1S,2S)-1-carbamoyl-2-methylcyclopropyl)-3-((methoxycarbonyl)amino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

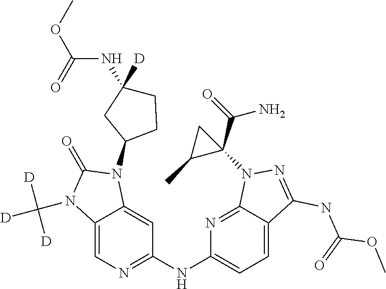 [SMILES: [2H]C([2H])([2H])n1c(=O)n([C@@H]2CC[C@@]([2H])(NC(=O)OC)C2)c2cc(Nc3ccc4c(NC(=O)OC)nn([C@@]5(C(N)=O)C[C@@H]5C)c4n3)ncc21]

This compound was prepared according to the procedures described in Example 30 with benzyl (1S,2S)-1-amino-2-methylcyclopropane-1-carboxylate hydrochloride replacing rac-methyl (1R,2S)-1-amino-2-(propan-2-yl)cyclopropane-1-carboxylate hydrochloride, and final purification by prep-LCMS (XBridge C18 column, eluting with acetonitrile/aqueous 0.1% NH₄OH, at flow rate of 60 mL/min). LCMS calculated for C₂₇H₂₉D₄N₁₀O₆(M+H)⁺: m/z=597.3; found 597.4. ¹H NMR (500 MHz, DMSO) δ 10.20 (s, 1H), 9.97 (s, 1H), 8.41 (s, 1H), 8.11 (d, J=8.9 Hz, 1H), 8.06 (s, 1H), 7.32 (s, 1H), 7.22 (s, 1H), 7.00 (d, J=9.0 Hz, 1H), 6.48 (s, 1H), 4.92-4.78 (m, 1H), 3.69 (s, 3H), 3.51 (s, 3H), 2.44 (dd, J=13.4, 7.6 Hz, 1H), 2.25-2.17 (m, 1H), 2.17-2.05 (m, 2H), 2.05-1.96 (m, 1H), 1.92 (dd, J=13.3, 9.3 Hz, 1H), 1.71 (dd, J=8.2, 4.7 Hz, 1H), 1.68-1.60 (m, 1H), 1.49 (dd, J=9.8, 4.6 Hz, 1H), 1.30 (d, J=6.3 Hz, 3H).

### Example 76. Methyl (1-((1S,2R)-1-carbamoyl-2-ethynylcyclopropyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate

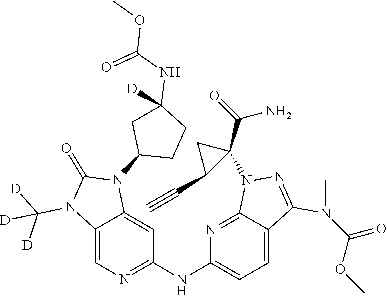 [SMILES: [2H]C([2H])([2H])n1c(=O)n([C@@H]2CC[C@@]([2H])(NC(=O)OC)C2)c2cc(Nc3ccc4c(N(C)C(=O)OC)nn([C@@]5(C(N)=O)C[C@@H]5C#C)c4n3)ncc21]

### Step 1. Methyl (1S,2R)-1-(6-chloro-3-iodo-1H-pyrazolo[3,4-b]pyridin-1-yl)-2-vinylcyclopropane-1-carboxylate

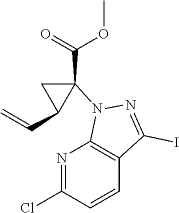 [SMILES: C=C[C@H]1C[C@]1(C(=O)OC)n1nc(I)c2ccc(Cl)nc21]

The title compound was prepared according to the procedure to convert the amino group to the iodo group in Example 54, step 9 by replacing benzyl (1S,2S)-1-(3-(bis(tert-butoxycarbonyl)amino)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate with methyl (1S,2R)-1-(3-amino-6-chloro-1H-pyrazolo[3,4-b]pyridin-1-yl)-2-vinylcyclopropane-1-carboxylate from example 11715, step 3. LCMS calculated for C₁₃H₁₂ClN₃O₂(M+H)⁺: m/z=404.0; found 404.0.

### Step 2. Methyl (1S,2R)-1-(6-chloro-3-((methoxycarbonyl)(methyl)amino)-1H-pyrazolo[3,4-b]pyridin-1-yl)-2-vinylcyclopropane-1-carboxylate

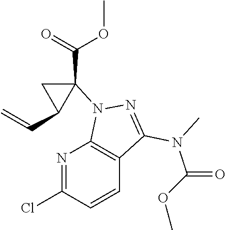 [SMILES: C=C[C@H]1C[C@]1(C(=O)OC)n1nc(N(C)C(=O)OC)c2ccc(Cl)nc21]

The title compound was prepared according to the procedure of Example 34, step 2 by replacing benzyl (1S,2S)-1-(6-chloro-3-iodo-1H-pyrazolo[3,4-b]pyridin-1-yl)-2-methylcyclopropane-1-carboxylate with methyl (1S,2R)-1-(6-chloro-3-iodo-1H-pyrazolo[3,4-b]pyridin-1-yl)-2-vinylcyclopropane-1-carboxylate and morpholine with methyl methylcarbamate. LCMS calculated for C₁₆H₁₈ClN₄O₄(M+H)⁺: m/z=365.1; found 365.1.

### Step 3. Methyl (1S,2S)-1-(6-chloro-3-((methoxycarbonyl)(methyl)amino)-1H-pyrazolo[3,4-b]pyridin-1-yl)-2-formylcyclopropane-1-carboxylate

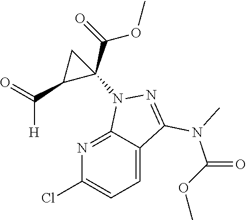 [SMILES: COC(=O)N(C)c1nn([C@@]2(C(=O)OC)C[C@@H]2C=O)c2nc(Cl)ccc12]

The title compound was prepared according to a similar procedure that was used in Example 38, step 3 using methyl (1S,2R)-1-(6-chloro-3-((methoxycarbonyl)(methyl)amino)-1H-pyrazolo[3,4-b]pyridin-1-yl)-2-vinylcyclopropane-1-carboxylate in place of methyl (1S,2R)-1-(bis(tert-butoxycarbonyl)amino)-2-vinylcyclopropane-1-carboxylate. The crude product was purified by flash column chromatography to give the title compound. LCMS calculated for C₁₅H₁₆ClN₄O₅(M+H)⁺: m/z=367.7; found 367.2.

### Step 4. Methyl (1S,2R)-1-(6-chloro-3-((methoxycarbonyl)(methyl)amino)-1H-pyrazolo[3,4-b]pyridin-1-yl)-2-ethynylcyclopropane-1-carboxylate

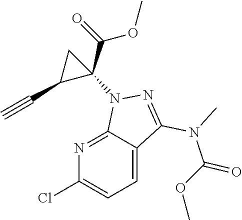 [SMILES: C#C[C@H]1C[C@]1(C(=O)OC)n1nc(N(C)C(=O)OC)c2ccc(Cl)nc21]

The title compound was prepared according to a similar procedure that was used in Example 38, step 4 using methyl (1S,2S)-1-(6-chloro-3-((methoxycarbonyl)(methyl)amino)-1H-pyrazolo[3,4-b]pyridin-1-yl)-2-formylcyclopropane-1-carboxylate in place of methyl (1S,2S)-1-(bis(tert-butoxycarbonyl)amino)-2-formylcyclopropane-1-carboxylate. The crude product was purified by flash column chromatography to give the title compound. LCMS calculated for C₁₆H₁₆ClN₄O₄(M+H)⁺: m/z=363.1; found 363.1.

### Step 5. Methyl (1S,2R)-2-ethynyl-1-(3-((methoxycarbonyl)(methyl)amino)-6-((1-((R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)cyclopropane-1-carboxylate

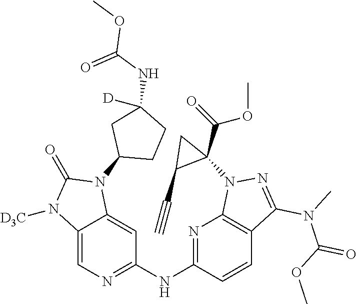 [SMILES: [2H]C([2H])([2H])n1c(=O)n([C@@H]2CC[C@@]([2H])(NC(=O)OC)C2)c2cc(Nc3ccc4c(N(C)C(=O)OC)nn([C@@]5(C(=O)OC)C[C@@H]5C#C)c4n3)ncc21]

The title compound was prepared according to a similar procedure that was used in Example 1, step 4 using methyl (1S,2R)-1-(6-chloro-3-((methoxycarbonyl)(methyl)amino)-1H-pyrazolo[3,4-b]pyridin-1-yl)-2-ethynylcyclopropane-1-carboxylate in place of tert-butyl 1-(6-chloro-4-(3,6-dihydro-2H-pyran-4-yl)-1H-pyrazolo[3,4-b]pyridin-1-yl)cyclopropane-1-carboxylate. The crude product was purified by flash column chromatography to give the title compound. LCMS calculated for C₃₀H₃₀D₄N₉O₇(M+H)⁺: m/z=636.3; found 636.2.

### Step 6. Methyl (1-((1S,2R)-1-carbamoyl-2-ethynylcyclopropyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-3-yl)(methyl)carbamate

The title compound was prepared according to a similar procedure that was used in Example 54, step 11 using methyl (1S,2R)-2-ethynyl-1-(3-((methoxycarbonyl)(methyl)amino)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)cyclopropane-1-carboxylate in place of benzyl (1S,2S)-1-(6-((1-((1R,3R)-3-((methoxycarbonyl)amino)-cyclopentyl-3-d)-3-(methyl-d₃)-2-oxo-2,3-dihydro-1H-imidazo[4,5-c]pyridin-6-yl)amino)-3-methyl-1H-pyrazolo[3,4-b]pyridin-1-yl)-2-methylcyclopropane-1-carboxylate. Final purification was done by prep-LCMS (XBridge C18 column, eluting with a gradient of acetonitrile in water containing 0.1% NH₄OH, at flow rate of 60 mL/min). LCMS calculated for C₂₉H₂₉D₄N₁₀O₆ (M+H)⁺: m/z=621.3; found 621.4. ¹H NMR (DMSO-d₆) δ: 10.02 (s, 1H), 8.22 (s, 1H), 8.07 (s, 1H), 7.91 (d, J=8.9 Hz, 1H), 7.40 (s, 1H), 7.20-7.15 (m, 2H), 6.72 (s, 1H), 4.90-4.84 (m, 1H), 3.73 (s, 3H), 3.53 (s, 3H), 3.39 (s, 3H), 2.94 (d, J=2.2 Hz, 1H), 2.66-2.60 (m, 1H), 2.46-2.41 (m, 1H), 2.24-2.19 (m, 1H), 2.17 (dd, J=7.9, 4.8 Hz, 1H), 2.15-2.07 (m, 2H), 1.92 (dd, J=13.4, 9.4 Hz, 1H), 1.82 (dd, J=9.9, 4.8 Hz, 1H), 1.64 (dt, J=12.4, 8.5 Hz, 1H).

### Example 77. Methyl ((1R,3R)-3-(6-((1-((1S,2R)-1-carbamoyl-2-ethynylcyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

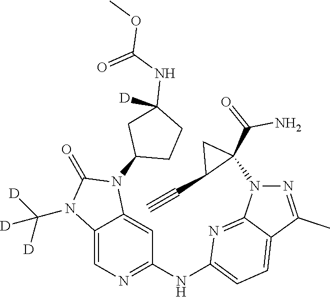 [SMILES: [2H]C([2H])([2H])n1c(=O)n([C@@H]2CC[C@@]([2H])(NC(=O)OC)C2)c2cc(Nc3ccc4c(C)nn([C@@]5(C(N)=O)C[C@@H]5C#C)c4n3)ncc21]

### Step 1. Methyl (1S,2R)-1-((tert-butoxycarbonyl)amino)-2-vinylcyclopropane-1-carboxylate

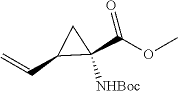

The title compound was prepared according to the procedures described in Example 42, step 1.

### Step 2. Methyl (1S,2R)-1-amino-2-vinylcyclopropane-1-carboxylate

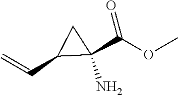

The title compound was prepared according to the procedures described in Example 43, step 2.

### Step 3. Methyl (1S,2R)-1-((6-chloro-3-cyanopyridin-2-yl)amino)-2-vinylcyclopropane-1-carboxylate

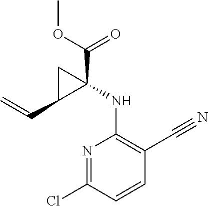 [SMILES: C=C[C@H]1C[C@@]1(Nc1nc(Cl)ccc1C#N)C(=O)OC]

The title compound was prepared according to a similar procedure that was used in Example 24, step 4 using methyl (1S,2R)-1-amino-2-vinylcyclopropane-1-carboxylate in place of isopropyl 1-amino-3-fluorocyclobutane-1-carboxylate and 6-chloro-2-fluoronicotinonitrile in place of 6-bromo-2-fluoronicotinonitrile. Purified by flash column chromatography to give the title compound as a colorless solid. LCMS calculated for C₁₃H₁₃ClN₃O₂(M+H)⁺: m/z=278.1; found 278.0.

### Step 4. Methyl (1S,2R)-1-(3-amino-6-chloro-1H-pyrazolo[3,4-b]pyridin-1-yl)-2-vinylcyclopropane-1-carboxylate

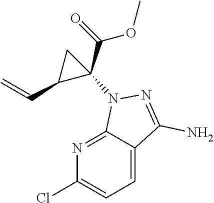 [SMILES: C=C[C@H]1C[C@]1(C(=O)OC)n1nc(N)c2ccc(Cl)nc21]

The title compound was prepared according to a similar procedure that was used in Example 24, step 5 using methyl (1S,2R)-1-((6-chloro-3-cyanopyridin-2-yl)amino)-2-vinylcyclopropane-1-carboxylate in place of isopropyl 1-((6-bromo-3-cyanopyridin-2-yl)amino)-3-fluorocyclobutane-1-carboxylate. Purified by flash column chromatography to give the title compound as a yellow tinted solid. LCMS calculated for C₁₃H₁₄ClN₄O₂ (M+H)⁺: m/z=293.1; found 293.1.

### Step 5. Methyl (1S,2R)-1-(3-(bis(tert-butoxycarbonyl)amino)-6-chloro-1H-pyrazolo[3,4-b]pyridin-1-yl)-2-vinylcyclopropane-1-carboxylate

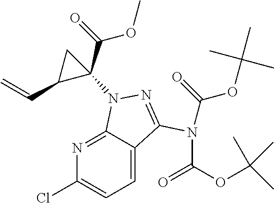 [SMILES: C=C[C@H]1C[C@]1(C(=O)OC)n1nc(N(C(=O)OC(C)(C)C)C(=O)OC(C)(C)C)c2ccc(Cl)nc21]

To a solution of methyl (1S,2R)-1-(3-amino-6-chloro-1H-pyrazolo[3,4-b]pyridin-1-yl)-2-vinylcyclopropane-1-carboxylate (1.77 g, 15.1 mmol) in THF (30 mL) was added triethylamine (2.10 mL, 15.1 mmol), Boc-anhydride (3.29 g, 15.1 mmol), and DMAP (0.737 g, 6.04 mmol). The mixture was stirred at ambient temperature for 30 min and was concentrated to a brown oil. The resulting crude oil was purified by flash column chromatography to give the desired product as a colorless foam (2.00 g, 67.0%). LCMS calculated for C₂₃H₂₉ClN₄O₆Na (M+Na)⁺: m/z=515.2; found 515.1.

### Step 6. Methyl (1S,2S)-1-(3-(bis(tert-butoxycarbonyl)amino)-6-chloro-1H-pyrazolo[3,4-b]pyridin-1-yl)-2-formylcyclopropane-1-carboxylate

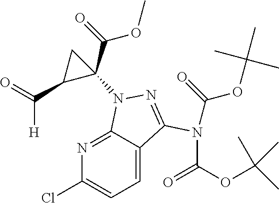 [SMILES: COC(=O)[C@]1(n2nc(N(C(=O)OC(C)(C)C)C(=O)OC(C)(C)C)c3ccc(Cl)nc32)C[C@@H]1C=O]

The title compound was prepared according to a similar procedure that was used in Example 38, step 3 using methyl (1S,2R)-1-(3-(bis(tert-butoxycarbonyl)amino)-6-chloro-1H-pyrazolo[3,4-b]pyridin-1-yl)-2-vinylcyclopropane-1-carboxylate in place of methyl (1S,2R)-1-(bis(tert-butoxycarbonyl)amino)-2-vinylcyclopropane-1-carboxylate. The crude product was purified by flash column chromatography to give the title compound as a colorless foam. LCMS calculated for C₂₂H₂₇ClN₄O₇Na (M+Na)⁺: m/z=517.2; found 517.1.

### Step 7. Methyl (1S,2R)-1-(3-((tert-butoxycarbonyl)amino)-6-chloro-1H-pyrazolo[3,4-b]pyridin-1-yl)-2-ethynylcyclopropane-1-carboxylate

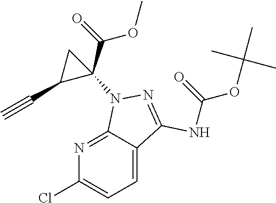 [SMILES: C#C[C@H]1C[C@]1(C(=O)OC)n1nc(NC(=O)OC(C)(C)C)c2ccc(Cl)nc21]

The title compound was prepared according to a similar procedure that was used in Example 38, step 4 using methyl (1S,2S)-1-(3-(bis(tert-butoxycarbonyl)amino)-6-chloro-1H-pyrazolo[3,4-b]pyridin-1-yl)-2-formylcyclopropane-1-carboxylate in place of methyl (1S,2S)-1-(bis(tert-butoxycarbonyl)amino)-2-formylcyclopropane-1-carboxylate. The crude product was purified by flash column chromatography to give the title compound as a colorless foam. LCMS calculated for C₁₈H₂₀ClN₄O₄(M+H)⁺: m/z=391.1; found 391.1.

### Step 8. Methyl (1S,2R)-1-(3-((tert-butoxycarbonyl)amino)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)-2-ethynylcyclopropane-1-carboxylate

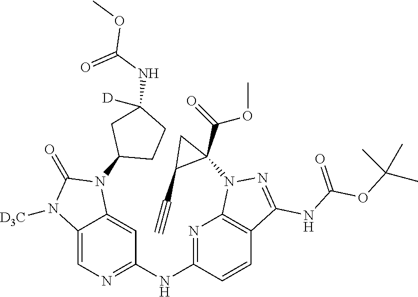 [SMILES: [2H]C([2H])([2H])n1c(=O)n([C@@H]2CC[C@@]([2H])(NC(=O)OC)C2)c2cc(Nc3ccc4c(NC(=O)OC(C)(C)C)nn([C@@]5(C(=O)OC)C[C@@H]5C#C)c4n3)ncc21]

The title compound was prepared according to a similar procedure that was used in Example 1, step 4 using methyl (1S,2R)-1-(3-((tert-butoxycarbonyl)amino)-6-chloro-1H-pyrazolo[3,4-b]pyridin-1-yl)-2-ethynylcyclopropane-1-carboxylate in place of tert-butyl 1-(6-chloro-4-(3,6-dihydro-2H-pyran-4-yl)-1H-pyrazolo[3,4-b]pyridin-1-yl)cyclopropane-1-carboxylate. The crude product was purified by flash column chromatography to give the title compound as a colorless foam. LCMS calculated for C₃₂H₃₄D₄N₉O₇(M+H)⁺: m/z=664.3; found 664.3.

### Step 9. Methyl (1S,2R)-2-ethynyl-1-(3-iodo-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)cyclopropane-1-carboxylate

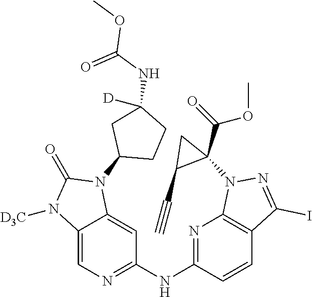 [SMILES: [2H]C([2H])([2H])n1c(=O)n([C@@H]2CC[C@@]([2H])(NC(=O)OC)C2)c2cc(Nc3ccc4c(I)nn([C@@]5(C(=O)OC)C[C@@H]5C#C)c4n3)ncc21]

To a flask containing methyl (1S,2R)-1-(3-((tert-butoxycarbonyl)amino)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-1-yl)-2-ethynylcyclopropane-1-carboxylate (98 mg, 0.15 mmol) was added DCM (1.1 mL) and TFA (0.5 mL). The reaction mixture was stirred at r.t. for 1 h and concentrated in vacuo. The residue was dilute with DCM and neutralized with sat. NaHCO₃ aq. solution. The product was extracted with DCM. The organic layers were combined, dried over Na₂SO₄, filtered, and the solvents were evaporated in vacuo. The residue was dissolved in MeCN (2 mL) and KI (23 mg, 0.14 mmol) and I₂ (70 mg, 0.3 mmol) were added. The mixture was heated to 40° C. and tert-butyl nitrite (0.065 mL, 0.55 mmol) was added. The vial was heated to 40° C. for 2 h. After cooling to r.t., the mixture was quenched with sat. Na₂S₂O₃ aq. solution followed by extraction with DCM. The organic phases were combined and washed with brine, dried over MgSO₄ and concentrated in vacuo. The crude material was purified by flash column chromatographyto give the desired product. LCMS calculated for C₂₇H₂₄D₄IN₈O₅(M+H)⁺: m/z=675.1; found 675.2.

### Steps 10 and 11. Methyl ((1R,3R)-3-(6-((1-((1S,2R)-1-carbamoyl-2-ethynylcyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate

The title compound was prepared according to the procedures described in Example 54, Steps 10-11 with methyl (1S,2R)-2-ethynyl-1-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclopropane-1-carboxylate replacing benzyl(1S,2S)-1-(3-iodo-6-((1-((1R,3R)-3-((methoxycarbonyl)amino)-cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate in Step 10 and final purification by prep-LCMS (XBridge C18 column, eluting with a gradient of acetonitrile in water containing 0.1% NH₄OH, at flow rate of 60 mL/min). LCMS calculated for C₂₇H₂₆D₄N₉O₄(M+H)⁺: m/z=548.3; found 548.4. ¹H NMR (DMSO-d₆) δ: 9.93 (s, 1H), 8.21 (s, 1H), 8.06 (s, 1H), 7.95 (d, J=8.8 Hz, 1H), 7.36 (s, 1H), 7.22-7.17 (m, 2H), 6.56 (s, 1H), 4.88-4.82 (m, 1H), 3.53 (s, 3H), 2.92 (d, J=2.3 Hz, 1H), 2.62-2.56 (m, 1H), 2.42 (s, 3H), 2.43-2.38 (m, 1H), 2.25-2.18 (m, 1H), 2.18-2.05 (m, 3H), 1.91 (dd, J=13.4, 9.4 Hz, 1H), 1.83-1.75 (m, 1H), 1.64 (dt, J=12.4, 8.5 Hz, 1H).

### Example 78. Methyl ((1R,3R)-3-(6-((1-((1R,2S)-2-carbamoyl-[1,1′-bi(cyclopropan)]-2-yl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

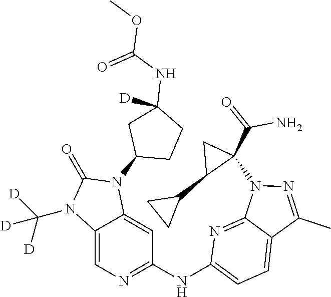 [SMILES: [2H]C([2H])([2H])n1c(=O)n([C@@H]2CC[C@@]([2H])(NC(=O)OC)C2)c2cc(Nc3ccc4c(C)nn([C@@]5(C(N)=O)C[C@@H]5C5CC5)c4n3)ncc21]

This compound was prepared according to the procedures described in Example 47 using methylzinc chloride in place of methyl-d3-zinc chloride. Final purification was done by prep-LCMS (Waters Sunfire column, eluting with a gradient of acetonitrile/water containing 0.1% TFA, at flow rate of 60 mL/min). LCMS calculated for C₂₈H₃₀D₄N₉O₄(M+H)⁺: m/z=564.3; found 564.4. ¹H NMR (600 MHz, DMSO) δ 11.28 (s, 1H), 8.24 (s, 1H), 8.16 (d, J=8.6 Hz, 1H), 7.81 (s, 1H), 7.39 (s, 1H), 7.34 (s, 1H), 6.96 (d, J=8.6 Hz, 1H), 6.86 (s, 1H), 5.01-4.98 (m, 1H), 2.46 (s, 3H), 2.37 (dd, J=13.6, 7.6 Hz, 1H), 2.25-2.12 (m, 2H), 2.08-2.00 (m, 1H), 1.98-1.91 (m, 2H), 1.71-1.63 (m, 2H), 1.43 (dd, J=9.8, 5.1 Hz, 1H), 1.05-0.98 (m, 1H), 0.58-0.49 (m, 2H), 0.40-0.28 (m, 2H).

### Example 79. Methyl-d₃ ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate

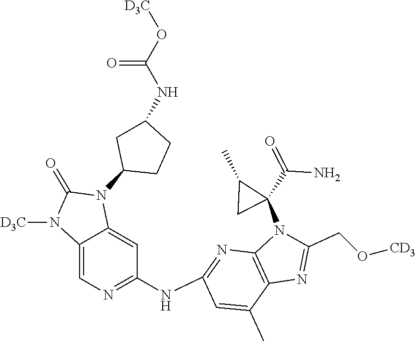 [SMILES: [2H]C([2H])([2H])OCc1nc2c(C)cc(Nc3cc4c(cn3)n(C([2H])([2H])[2H])c(=O)n4[C@@H]3CC[C@@H](NC(=O)OC([2H])([2H])[2H])C3)nc2n1[C@@]1(C(N)=O)C[C@@H]1C]

This compound was prepared according to the procedures described in Example 83 step 1-7 using 2-(methoxy-d3)acetic acid (Enamine EN300-7572628; chemical purity 95%) in place of 2-methoxyacetic acid and methyl-d3 ((1R,3R)-3-(6-amino-3-(methyl-d3)-2-oxo-2,3-dihydro-1H-imidazo[4,5-c]pyridin-1-yl)cyclopentyl)carbamate in place of methyl ((1R,3R)-3-(6-amino-3-(methyl-d3)-2-oxo-2,3-dihydro-1H-imidazo[4,5-c]pyridin-1-yl)cyclopentyl-1-d)carbamate. LCMS calculated for C₂₈H₂₇D₉N₉O₅(M+H)⁺: m/z=587.3; found 587.3

### Example 80. Methyl-d3 ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate

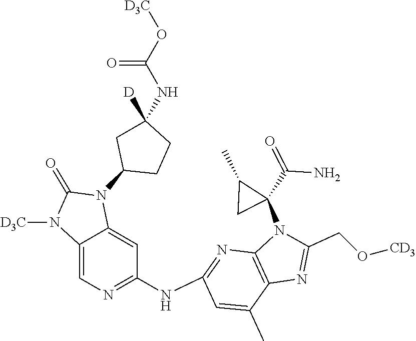 [SMILES: [2H]C([2H])([2H])OCc1nc2c(C)cc(Nc3cc4c(cn3)n(C([2H])([2H])[2H])c(=O)n4[C@@H]3CC[C@@]([2H])(NC(=O)OC([2H])([2H])[2H])C3)nc2n1[C@@]1(C(N)=O)C[C@@H]1C]

This compound was prepared according to the procedures described in Example 83 step 1-7 using 2-(methoxy-d3)acetic acid (Enamine EN300-7572628; chemical purity 95%) in place of 2-methoxyacetic acid and methyl-d3 ((1R,3R)-3-(6-amino-3-(methyl-d3)-2-oxo-2,3-dihydro-1H-imidazo[4,5-c]pyridin-1-yl)cyclopentyl-1-d)carbamate in place of methyl ((1R,3R)-3-(6-amino-3-(methyl-d3)-2-oxo-2,3-dihydro-1H-imidazo[4,5-c]pyridin-1-yl)cyclopentyl-1-d)carbamate. LCMS calculated for C₂₈H₂₆D₁₀N₉O₅(M+H)⁺: m/z=588.3; found 588.3. ¹H NMR (500 MHz, DMSO) δ 11.33 (brs, 1H), 8.28 (s, 1H), 7.44-7.36 (m, 2H), 7.33 (s, 1H), 7.16 (s, 1H), 6.92 (s, 1H), 5.00-4.85 (m, 1H), 4.75 (d, J=12.5 Hz, 1H), 4.67 (d, J=12.7 Hz, 1H), 2.60 (s, 3H), 2.33 (dd, J=13.7, 7.6 Hz, 1H), 2.16-2.24 (m, 1H), 2.11-2.16 (m, 1H), 2.09-2.01 (m, 1H), 1.94 (dd, J=13.7, 9.6 Hz, 1H), 1.67-1.61 (m, 1H), 1.56 (s, 1H), 1.38 (d, J=6.3 Hz, 3H).

### Example 81. Methyl ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate

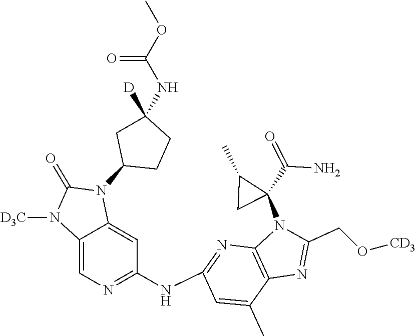 [SMILES: [2H]C([2H])([2H])OCc1nc2c(C)cc(Nc3cc4c(cn3)n(C([2H])([2H])[2H])c(=O)n4[C@@H]3CC[C@@]([2H])(NC(=O)OC)C3)nc2n1[C@@]1(C(N)=O)C[C@@H]1C]

This compound was prepared according to the procedures described in Example 83 step 1-7 using 2-(methoxy-d3)acetic acid (Enamine EN300-7572628; chemical purity 95%) in place of 2-methoxyacetic acid. LCMS calculated for C₂₈H₂₉D₇N₉O₅(M+H)⁺: m/z=585.3; found 585.3.

### Example 82. Methyl-d3 ((1R,3R)-3-(6-((3-((1S,2S)-1-carbamoyl-2-methylcyclopropyl)-2-(methoxymethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate

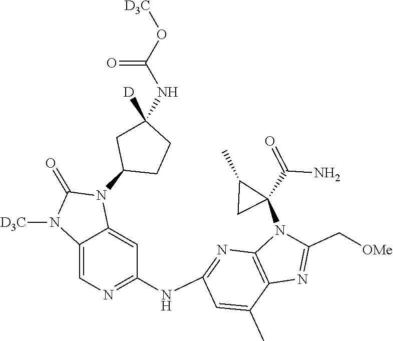 [SMILES: [2H]C([2H])([2H])OC(=O)N[C@]1([2H])CC[C@@H](n2c(=O)n(C([2H])([2H])[2H])c3cnc(Nc4cc(C)c5nc(COC)n([C@@]6(C(N)=O)C[C@@H]6C)c5n4)cc32)C1]

This compound was prepared according to the procedures described in Example 83 step 1-7 using methyl-d3 ((1R,3R)-3-(6-amino-3-(methyl-d3)-2-oxo-2,3-dihydro-1H-imidazo[4,5-c]pyridin-1-yl)cyclopentyl-1-d)carbamate in place of methyl ((1R,3R)-3-(6-amino-3-(methyl-d₃)-2-oxo-2,3-dihydro-1H-imidazo[4,5-c]pyridin-1-yl)cyclopentyl-1-d)carbamate. LCMS calculated for C₂₈H₂₉D₇N₉O₅(M+H)⁺: m/z=585.3; found 585.3

### Example 83. Methyl ((1R,3R)-3-(6-((3-((1S,2S)-1-carbamoyl-2-methylcyclopropyl)-2-(methoxymethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

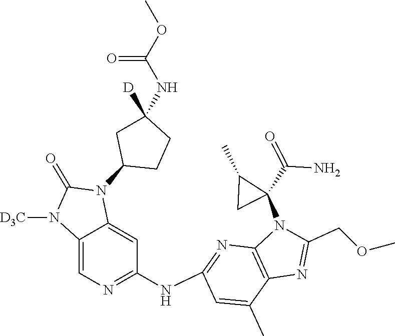 [SMILES: [2H]C([2H])([2H])n1c(=O)n([C@@H]2CC[C@@]([2H])(NC(=O)OC)C2)c2cc(Nc3cc(C)c4nc(COC)n([C@@]5(C(N)=O)C[C@@H]5C)c4n3)ncc21]

### Step 1. Benzyl (1S,2S)-1-((6-chloro-4-methyl-3-nitropyridin-2-yl)amino)-2-methylcyclopropane-1-carboxylate

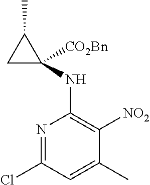 [SMILES: Cc1cc(Cl)nc(N[C@@]2(C(=O)OCc3ccccc3)C[C@@H]2C)c1[N+](=O)[O-]]

To a flask containing benzyl (1S,2S)-1-amino-2-methylcyclopropane-1-carboxylate hydrochloride (7.0 g, 29.0 mmol) and 2,6-dichloro-4-methyl-3-nitropyridine (6.1 g, 29.5 mmol) in DMSO (15 mL) was added DIPEA (15 mL, 87 mmol). The reaction was heated at 65° C. for 1 h. The mixture was diluted with DCM and washed with water. The organic phase was dried over MgSO₄ and concentrated in vacuo. The crude material was purified by Biotage Isolera, eluting with 0-35% EtOAc in hexanes, to give the desired product (9.2 g, 85% yield) as a yellow solid. LCMS calculated for C₁₈H₁₉ClN₃O₄(M+H)⁺: m/z=376.1; found: 376.2

### Step 2. Benzyl (1S,2S)-1-((3-amino-6-chloro-4-methylpyridin-2-yl)amino)-2-methylcyclopropane-1-carboxylate

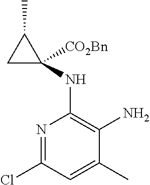 [SMILES: Cc1cc(Cl)nc(N[C@@]2(C(=O)OCc3ccccc3)C[C@@H]2C)c1N]

The title compound was prepared according to the procedures described in Intermediate A, Step 6, with benzyl (1S,2S)-1-((6-chloro-4-methyl-3-nitropyridin-2-yl)amino)-2-methylcyclopropane-1-carboxylate replacing tert-butyl ((1R,3R)-3-((2-chloro-5-nitropyridin-4-yl)amino)cyclopentyl-1-d)carbamate. LCMS calculated for C₁₈H₂₁ClN₃O₂ (M+H)⁺: m/z=346.1; found: 346.2

### Step 3. Benzyl (1S,2S)-1-((6-chloro-3-(2-methoxyacetamido)-4-methylpyridin-2-yl)amino)-2-methylcyclopropane-1-carboxylate

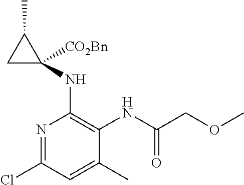 [SMILES: COCC(=O)Nc1c(C)cc(Cl)nc1N[C@@]1(C(=O)OCc2ccccc2)C[C@@H]1C]

To a vial containing benzyl (1S,2S)-1-((3-amino-6-chloro-4-methylpyridin-2-yl)amino)-2-methylcyclopropane-1-carboxylate (0.63 g, 1.8 mmol), 2-methoxyacetic acid (0.25 g, 2.7 mmol) in DMF (3.0 mL) was added DIPEA (1.0 mL, 5.7 mmol) and HATU (1.04 g, 2.73 mmol). After stirring at 40° C. overnight, the mixture was diluted with DCM and washed with water. The organic phase was dried over MgSO₄ and concentrated in vacuo. The crude material was purified by Biotage Isolera, eluting with 0-80% EtOAc in hexanes, to give the desired product (0.75 g, 99% yield) as white solid. LCMS calculated for C₂₁H₂₅ClN₃O₄ (M+H)⁺: m/z=418.2; found: 418.2

### Step 4. Benzyl (1S,2S)-1-(5-chloro-2-(methoxymethyl)-7-methyl-3H-imidazo[4,5-b]pyridin-3-yl)-2-methylcyclopropane-1-carboxylate

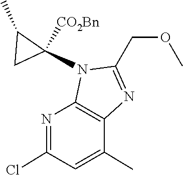

To a vial containing benzyl (1S,2S)-1-((6-chloro-3-(2-methoxyacetamido)-4-methylpyridin-2-yl)amino)-2-methylcyclopropane-1-carboxylate (0.75 g, 1.8 mmol) in acetic acid (5.0 mL) was added TSA (40 mg). The reaction mixture was sealed and heated at 120° C. overnight. After cooling down to r.t., the reaction mixture was concentrated in vacuo and diluted with DCM (5 mL). The pH of the mixture was adjusted to ˜10 using ammonia aqueous solution, followed by extraction with DCM (2×20 mL). The organic layers were combined, dried over MgSO₄ and concentrated in vacuo. The crude material was purified by Biotage Isolera, eluting with 0-70% EtOAc in hexanes, to give the desired product (0.41 g 57% yield) of white solid. LCMS calculated for C₂₁H₂₃ClN₃O₃(M+H)⁺: m/z=400.2; found: 400.2

### Step 5. Benzyl (1S,2S)-1-(5-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-2-(methoxymethyl)-7-methyl-3H-imidazo[4,5-b]pyridin-3-yl)-2-methylcyclopropane-1-carboxylate

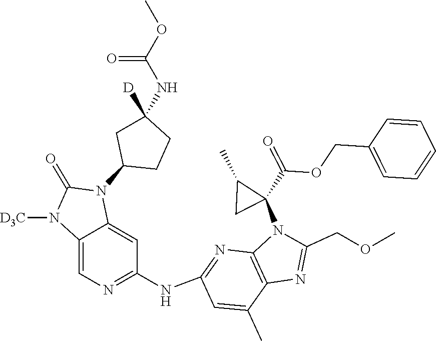 [SMILES: [2H]C([2H])([2H])n1c(=O)n([C@@H]2CC[C@@]([2H])(NC(=O)OC)C2)c2cc(Nc3cc(C)c4nc(COC)n([C@@]5(C(=O)OCc6ccccc6)C[C@@H]5C)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (0.317 g, 1.02 mmol), benzyl (1S,2S)-1-(5-chloro-2-(methoxymethyl)-7-methyl-3H-imidazo[4,5-b]pyridin-3-yl)-2-methylcyclopropane-1-carboxylate (0.410 g, 1.02 mmol), cesium carbonate (1.00 g, 3.08 mmol), XantPhos (0.132 g, 0.23 mmol), and Pd₂(dba)₃ (0.105 g, 0.11 mmol) was evacuated and backfilled with nitrogen three times, followed by the addition of anhydrous dioxane (5 mL). The vial was sealed and heated to 110° C. for 16 h. After cooling to r.t., the mixture was filtered and concentrated in vacuo. The residue was purified by Biotage Isolera, eluting with 0-20% MeOH in DCM, to give the desired product (0.45 g, 66% yield) of white solid. LCMS calculated for C₃₅H₃₇D₄N₈O₆(M+H)⁺: m/z=673.4; found: 673.4

### Step 6. (1S,2S)-1-(5-((1-((1R,3R)-3-((Methoxycarbonyl)amino)cyclopentyl-3-d)-3-(methyl-d3)-2-oxo-2,3-dihydro-1H-imidazo[4,5-c]pyridin-6-yl)amino)-2-(methoxymethyl)-7-methyl-3H-imidazo[4,5-b]pyridin-3-yl)-2-methylcyclopropane-1-carboxylic acid

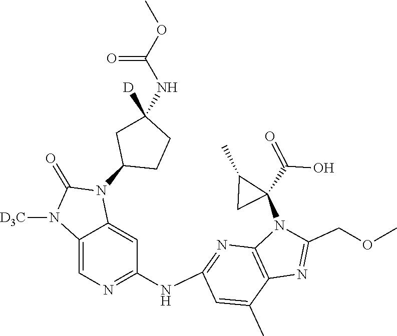 [SMILES: [2H]C([2H])([2H])n1c(=O)n([C@@H]2CC[C@@]([2H])(NC(=O)OC)C2)c2cc(Nc3cc(C)c4nc(COC)n([C@@]5(C(=O)O)C[C@@H]5C)c4n3)ncc21]

To a vial containing benzyl (1S,2S)-1-(5-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-2-(methoxymethyl)-7-methyl-3H-imidazo[4,5-b]pyridin-3-yl)-2-methylcyclopropane-1-carboxylate (0.45 g, 0.67 mmol) was added methanol/THF/1N NaOH water solution (6.0 mL, v/v/v=1/1/1). After heating at 65° C. for 3 h, the reaction mixture was cooled to r.t. and diluted with CH₂Cl₂ (10 mL). The pH of the mixture was adjusted to ˜4 using HCl aqueous solution, followed by extraction with CH₂Cl₂ (3×10 mL). The organic layers were combined, dried over MgSO₄ and concentrated in vacuo. The crude material was used in the next step without further purification. LCMS calculated for C₂₈H₃₁D₄N₈O₆(M+H)⁺: m/z=583.3; found: 583.4.

### Step 7. Methyl ((1R,3R)-3-(6-((3-((1S,2S)-1-carbamoyl-2-methylcyclopropyl)-2-(methoxymethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate

The title compound was prepared according to the procedures described in Example 48, Step 5, with (1S,2S)-1-(5-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-2-(methoxymethyl)-7-methyl-3H-imidazo[4,5-b]pyridin-3-yl)-2-methylcyclopropane-1-carboxylic acid replacing (1R,2S)-2-(3-methoxy-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-[1,1′-bi(cyclopropane)]-2-carboxylic acid. LCMS calculated for C₂₈H₃₂D₄N₉O₅(M+H)⁺: m/z=582.3; found 582.3. ¹H NMR (500 MHz, DMSO) δ 11.34 (brs, 1H), 8.28 (s, 1H), 7.48-7.40 (m, 2H), 7.33 (s, 1H), 7.16 (s, 1H), 6.92 (s, 1H), 5.00-4.86 (m, 1H), δ 4.75 (d, J=12.6 Hz, 1H), 4.67 (d, J=12.8 Hz, 1H), 3.55 (s, 3H), 3.44 (s, 3H), 2.59 (s, 3H), 2.33 (dd, J=13.7, 7.6 Hz, 1H), 2.25-2.17 (m, 1H), 2.16-2.09 (m, 1H), 2.09-1.99 (m, 1H), 1.94 (dd, J=13.6, 9.6 Hz, 1H), 1.68-1.60 (m, 1H), 1.56 (s, 1H), 1.38 (d, J=6.2 Hz, 3H).

### Example 84. Methyl ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate

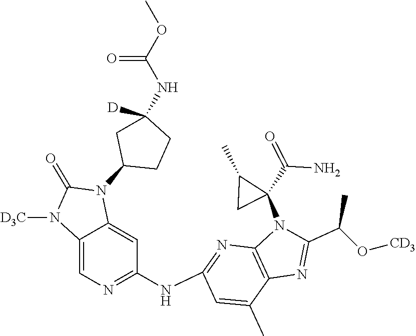 [SMILES: [2H]C([2H])([2H])O[C@H](C)c1nc2c(C)cc(Nc3cc4c(cn3)n(C([2H])([2H])[2H])c(=O)n4[C@@H]3CC[C@@]([2H])(NC(=O)OC)C3)nc2n1[C@@]1(C(N)=O)C[C@@H]1C]

### Step 1. (R)-2-(Methoxy-d₃)propanoic acid

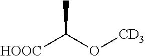

Sodium hydride (2.0 g, 60% in mineral oil, 50.0 mmol) was washed with hexane (20 mL) and slowly added in portions into methanol-d4 (20 mL, D %, ≥99.8%) at 0° C. in an ice bath. After the hydrogen gas bubbling ceased, the reaction mixture was allowed to stir at room temperature for an additional 15 min. The resulted solution was mixed with (S)-2-bromopropanoic acid (3.0 g, 19.7 mmol) and stirred at room temperature overnight. The reaction mixture was concentrated in vacuo and diluted with CH₂Cl₂ (50 mL). The pH of the mixture was adjusted to ˜4 using HCl aqueous solution, followed by extraction with CH₂Cl₂ (3×10 mL). The organic layers were combined, dried over MgSO₄ and concentrated in vacuo. The crude material (2.0 g, 94% yield) was used in the next step without further purification.

### Step 2. Methyl ((1R,3R)-3-(6-((3-((1S,2S)-1-carbamoyl-2-methylcyclopropyl)-2-((R)-1-(methoxy-d₃)ethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

This compound was prepared according to the procedures described in Example 81 using (R)-2-(methoxy-d3)propanoic acid in place of 2-(methoxy-d3)acetic acid. LCMS calculated for C₂₉H₃₁D₇N₉O₅ (M+H)⁺: m/z=599.3; found 599.3

### Example 85. Methyl-d3 ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl)carbamate

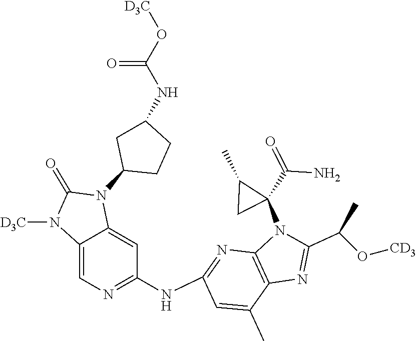 [SMILES: [2H]C([2H])([2H])OC(=O)N[C@@H]1CC[C@@H](n2c(=O)n(C([2H])([2H])[2H])c3cnc(Nc4cc(C)c5nc([C@@H](C)OC([2H])([2H])[2H])n([C@@]6(C(N)=O)C[C@@H]6C)c5n4)cc32)C1]

This compound was prepared according to the procedures described in Example 84 using methyl-d₃ ((1R,3R)-3-(6-amino-3-(methyl-d₃)-2-oxo-2,3-dihydro-1H-imidazo[4,5-c]pyridin-1-yl)cyclopentyl)carbamate in place of methyl ((1R,3R)-3-(6-amino-3-(methyl-d3)-2-oxo-2,3-dihydro-1H-imidazo[4,5-c]pyridin-1-yl)cyclopentyl-1-d)carbamate. LCMS calculated for C₂₉H₂₉D₉N₉O₅(M+H)⁺: m/z=601.3; found 601.3

### Example 86. Methyl-d3 ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate

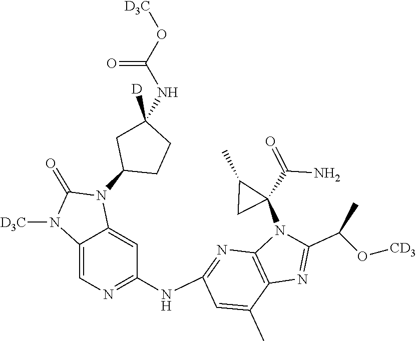 [SMILES: [2H]C([2H])([2H])OC(=O)N[C@]1([2H])CC[C@@H](n2c(=O)n(C([2H])([2H])[2H])c3cnc(Nc4cc(C)c5nc([C@@H](C)OC([2H])([2H])[2H])n([C@@]6(C(N)=O)C[C@@H]6C)c5n4)cc32)C1]

This compound was prepared according to the procedures described in Example 84 using methyl-d₃ ((1R,3R)-3-(6-amino-3-(methyl-d3)-2-oxo-2,3-dihydro-1H-imidazo[4,5-c]pyridin-1-yl)cyclopentyl-1-d)carbamate in place of methyl ((1R,3R)-3-(6-amino-3-(methyl-d3)-2-oxo-2,3-dihydro-1H-imidazo[4,5-c]pyridin-1-yl)cyclopentyl-1-d)carbamate. LCMS calculated for C₂₉H₂₈D₁₀N₉O₅(M+H)⁺: m/z=602.3; found 602.3 ¹H NMR (500 MHz, DMSO) δ 10.86 (brs, 1H), 8.15 (s, 1H), 7.49 (s, 1H), 7.08 (s, 1H), 6.94 (s, 1H), 4.99-4.84 (m, 2H), 2.59 (s, 3H), 2.38 (dd, J=13.6, 7.6 Hz, 1H), 2.27-2.19 (m, 2H), 2.01-1.93 (m, 2H), 1.73-1.63 (m, 4H), 1.47 (s, 1H), 1.40 (d, J=6.2 Hz, 3H).

### Example 87. Methyl ((1R,3R)-3-(6-((3-((1S,2S)-1-carbamoyl-2-methylcyclopropyl)-2-((R)-1-methoxyethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate

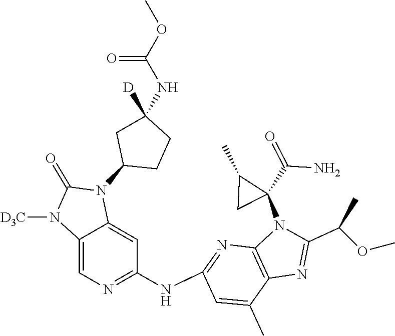 [SMILES: [2H]C([2H])([2H])n1c(=O)n([C@@H]2CC[C@@]([2H])(NC(=O)OC)C2)c2cc(Nc3cc(C)c4nc([C@@H](C)OC)n([C@@]5(C(N)=O)C[C@@H]5C)c4n3)ncc21]

This compound was prepared according to the procedures described in Example 83 using (R)-2-methoxypropanoic acid in place of 2-methoxyacetic acid. LCMS calculated for C₂₉H₃₄D₄N₉O₅(M+H)⁺: m/z=596.3; found 596.3. ¹H NMR (500 MHz, DMSO, 70° C.) δ 10.71 (brs, 1H), 8.12 (s, 1H), 7.52 (s, 1H), 7.04 (s, 1H), 6.95 (s, 1H), 5.07-4.84 (m, 2H), 3.57 (s, 3H), 3.42 (s, 3H), 2.59 (s, 3H), 2.39 (dd, J=13.7, 7.7 Hz, 1H), 2.29-2.05 (m, 4H), 2.01-1.93 (m, 2H), 1.73-1.63 (m, 4H), 1.49-1.45 (m, 1H), 1.41 (d, J=6.3 Hz, 3H).

### Example 88-89

The compounds of Examples 88-89 in Table 4 were prepared according to the procedures described in Example 93, with CD₃OD (D %≥99.8%) and CH₃OH replacing EtOH in step 4.

TABLE 4

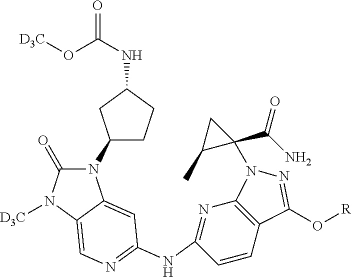

| Example | R | LCMS |
|---|---|---|
| 88 | CD₃ | calculated for C₂₆H₂₃D₉N₉O₅ (M + H)⁺: m/z = 559.3; found 559.3 |
| 89 | CH₃ | calculated for C₂₆H₂₆D₆N₉O₅ (M + H)⁺: m/z = 556.3; found 556.4 |

### Example 90. Methyl-d₃ ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

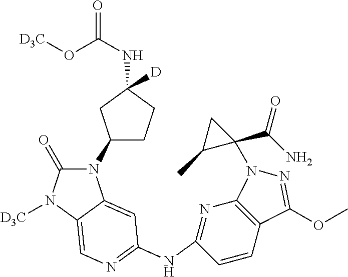 [SMILES: [2H]C([2H])([2H])OC(=O)N[C@]1([2H])CC[C@@H](n2c(=O)n(C([2H])([2H])[2H])c3cnc(Nc4ccc5c(OC)nn([C@@]6(C(N)=O)C[C@@H]6C)c5n4)cc32)C1]

### Step 1. Methyl-d₃ ((1R,3R)-3-(6-amino-3-(methyl-d₃)-2-oxo-2,3-dihydro-1H-imidazo[4,5-c]pyridin-1-yl)cyclopentyl-1-d)carbamate

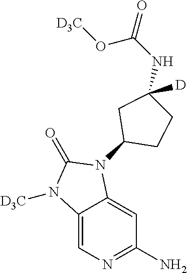 [SMILES: [2H]C([2H])([2H])OC(=O)N[C@]1([2H])CC[C@@H](n2c(=O)n(C([2H])([2H])[2H])c3cnc(N)cc32)C1]

A microwave vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (500 mg, 1.62 mmol) and cesium carbonate (1.3 g, 4.04 mmol) in CD₃OD (10 mL) was irradiated at 110° C. for 1 h. The reaction mixture was cooled to r.t. and solvent was evaporated. The crude was purified by Combi-Flash (MeOH/DCM) to obtain desired product (392 mg, 1.05 mmol, 65% yield). LCMS calculated for C₁₄H₁₃D₇N₅O₃(M+H)⁺: m/z=313.2; found: 313.3.

### Step 2. (1S,2S)-1-(6-Bromo-3-methoxy-1H-pyrazolo[3,4-b]pyridin-1-yl)-2-methylcyclopropane-1-carboxamide

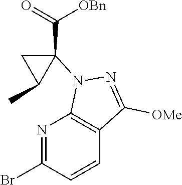 [SMILES: COc1nn([C@@]2(C(=O)OCc3ccccc3)C[C@@H]2C)c2nc(Br)ccc12]

The title compound was prepared according to the procedures described in Example 93, Steps 1-6, with CH₃OH replacing EtOH in Step 4. LCMS calculated for C₁₂H₁₄BrN₄O₂ (M+H)⁺: m/z=325.0; found 325.0.

### Step 3. Methyl-d₃ ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 93, Step 7, with methyl-d₃ ((1R,3R)-3-(6-amino-3-(methyl-d₃)-2-oxo-2,3-dihydro-1H-imidazo[4,5-c]pyridin-1-yl)cyclopentyl-1-d)carbamate and (1S,2S)-1-(6-bromo-3-methoxy-1H-pyrazolo[3,4-b]pyridin-1-yl)-2-methylcyclopropane-1-carboxamide replacing methyl-d₃ ((1R,3R)-3-(6-amino-3-(methyl-d₃)-2-oxo-2,3-dihydro-1H-imidazo[4,5-c]pyridin-1-yl)cyclopentyl)carbamate and (1S,2S)-1-(6-bromo-3-ethoxy-1H-pyrazolo[3,4-b]pyridin-1-yl)-2-methylcyclopropane-1-carboxamide. LCMS calculated for C₂₆H₂₅D₇N₉O₅(M+H)⁺: m/z=557.3; found 557.3. ¹H NMR (600 MHz, DMSO-d₆) δ 10.99 (s, 1H), 8.22 (s, 1H), 8.01 (d, J=8.6 Hz, 1H), 7.84 (s, 1H), 7.37-7.25 (m, 2H), 6.92 (d, J=8.6 Hz, 1H), 6.77 (s, 1H), 4.96-4.86 (m, 1H), 4.01 (s, 3H), 2.43-2.33 (m, 1H), 2.24-2.01 (m, 4H), 1.97-1.91 (m, 1H), 1.79-1.59 (m, 2H), 1.51-1.45 (m, 1H), 1.32 (d, J=6.3 Hz, 3H) ppm.

### Example 91. Methyl ((1R,3R)-3-(6-((1-((1S,2S)-1-carbamoyl-2-methylcyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

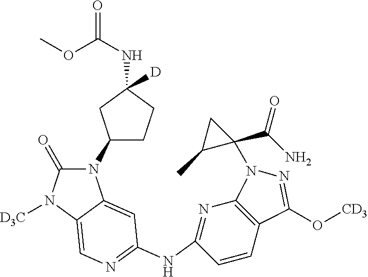 [SMILES: [2H]C([2H])([2H])Oc1nn([C@@]2(C(N)=O)C[C@@H]2C)c2nc(Nc3cc4c(cn3)n(C([2H])([2H])[2H])c(=O)n4[C@@H]3CC[C@@]([2H])(NC(=O)OC)C3)ccc12]

The title compound was prepared according to the procedures described in Example 53, with CD₃OD (D %≥99.8%) replacing EtOH in Step 10. LCMS calculated for C₂₆H₂₅D₇N₉O₅(M+H)⁺: m/z=557.3; found 557.3. ¹H NMR (500 MHz, DMSO-d₆) δ 11.07 (s, 1H), 8.23 (s, 1H), 8.02 (d, J=8.6 Hz, 1H), 7.84 (s, 1H), 7.39-7.27 (m, 2H), 6.92 (d, J=8.6 Hz, 1H), 6.77 (s, 1H), 4.98-4.87 (m, 1H), 3.54 (s, 3H), 2.39-2.33 (m, 1H), 2.27-2.02 (m, 4H), 1.98-1.89 (m, 1H), 1.80-1.61 (m, 2H), 1.51-1.45 (m, 1H), 1.32 (d, J=6.3 Hz, 3H) ppm.

### Example 92. Methyl-d₃ ((1R,3R)-3-(6-((1-((1S,2S)-1-carbamoyl-2-methylcyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

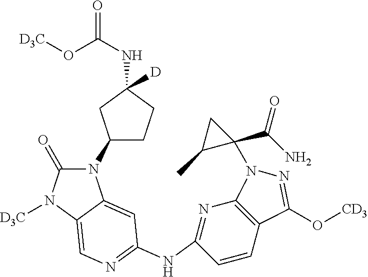 [SMILES: [2H]C([2H])([2H])OC(=O)N[C@]1([2H])CC[C@@H](n2c(=O)n(C([2H])([2H])[2H])c3cnc(Nc4ccc5c(OC([2H])([2H])[2H])nn([C@@]6(C(N)=O)C[C@@H]6C)c5n4)cc32)C1]

### Step 1. (1S,2S)-1-(3-(Methoxy-d₃)-6-((1-((1R,3R)-3-(((methoxy-d₃)carbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid

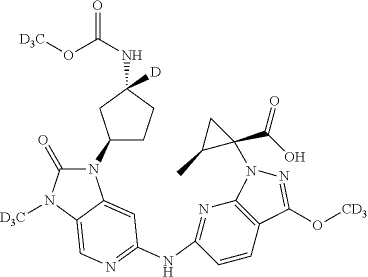 [SMILES: [2H]C([2H])([2H])OC(=O)N[C@]1([2H])CC[C@@H](n2c(=O)n(C([2H])([2H])[2H])c3cnc(Nc4ccc5c(OC([2H])([2H])[2H])nn([C@@]6(C(=O)O)C[C@@H]6C)c5n4)cc32)C1]

A mixture of benzyl (1S,2S)-1-(3-iodo-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate (200 mg, 0.27 mmol), copper(I) iodide (10 mg, 0.054 mmol), 3,4,7,8-tetramethyl-1,10-phenanthroline (26 mg, 0.108 mmol), and cesium carbonate (352 mg, 1.08 mmol) was combined in CD₃OD (2 mL; D %≥99.8%) in a microwave vial and irradiated at 100° C. for 4 h. The reaction was then cooled to r.t. and diluted with MeOH, filtered through celite and concentrated. The obtained product was used directly in the next step. LCMS calculated for C₂₆H₂₁D₁₀NO₆ (M+H)⁺: m/z=561.3; found 561.3.

### Step 2. Methyl-d₃ ((1R,3R)-3-(6-((1-((1S,2S)-1-carbamoyl-2-methylcyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 53, Step 11 with (1S,2S)-1-(3-(Methoxy-d₃)-6-((1-((1R,3R)-3-(((methoxy-d₃)carbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid replacing (1S,2S)-1-(3-ethoxy-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid. LCMS calculated for C₂₆H₂₂D₁₀N₉O₅(M+H)⁺: m/z=560.3; found 560.3. ¹H NMR (600 MHz, DMSO-d₆) δ 11.06 (s, 1H), 8.23 (s, 1H), 8.02 (d, J=8.5 Hz, 1H), 7.83 (s, 1H), 7.37-7.25 (m, 2H), 6.92 (d, J=8.6 Hz, 1H), 6.77 (s, 1H), 4.96-4.86 (m, 1H), 2.39-2.33 (m, 1H), 2.24-2.00 (m, 4H), 1.97-1.91 (m, 1H), 1.79-1.73 (m, 1H), 1.68-1.62 (m, 1H), 1.50-1.47 (m, 1H), 1.32 (d, J=6.3 Hz, 3H) ppm.

### Example 93. Methyl-d₃ ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate

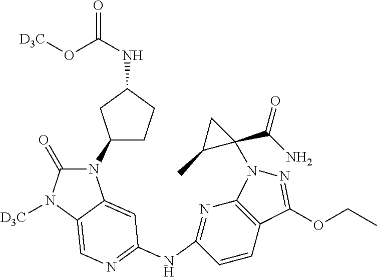 [SMILES: [2H]C([2H])([2H])OC(=O)N[C@@H]1CC[C@@H](n2c(=O)n(C([2H])([2H])[2H])c3cnc(Nc4ccc5c(OCC)nn([C@@]6(C(N)=O)C[C@@H]6C)c5n4)cc32)C1]

### Step 1. Ethyl (1S,2S)-1-((6-bromo-3-cyanopyridin-2-yl)amino)-2-methylcyclopropane-1-carboxylate

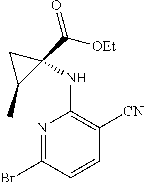 [SMILES: CCOC(=O)[C@]1(Nc2nc(Br)ccc2C#N)C[C@@H]1C]

To a flask containing 6-bromo-2-fluoronicotinonitrile (30 g, 148 mmol) and ethyl (1S,2S)-1-amino-2-methylcyclopropane-1-carboxylate (21 g, 148 mmol) in DMSO (135 mL) was added DIPEA (39 mL, 223 mmol). After heating at 65° C. for 20 h, the reaction mixture was cooled to r.t. and added to iced water. The precipitated solid was filtered. The obtained solid was then dissolved in DCM, dried over MgSO₄, filtered and concentrated. The material was then stirred with hexanes, filtered and the obtained solid was dried to yield the desired product (32 g, 99 mmol, 67% yield). LCMS calculated for C₁₃H₁₅BrN₃O₂(M+H)⁺: m/z=324.0; found 324.0.

### Step 2. Ethyl (1S,2S)-1-(3-amino-6-bromo-1H-pyrazolo[3,4-b]pyridin-1-yl)-2-methylcyclopropane-1-carboxylate

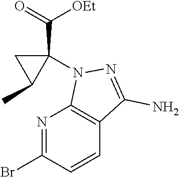 [SMILES: CCOC(=O)[C@]1(n2nc(N)c3ccc(Br)nc32)C[C@@H]1C]

To a flask containing ethyl (1S,2S)-1-((6-bromo-3-cyanopyridin-2-yl)amino)-2-methylcyclopropane-1-carboxylate (10 g, 30.8 mmol) in THF (51 mL) and DMF (100 mL) at 0° C. was slowly added LHMDS (31 mL, 46.3 mmol, 1.5 M in THF). After stirring at 0° C. for 10 min, (aminooxy)diphenylphosphine oxide (11 g, 46.3 mmol) was added to the reaction mixture. The reaction was allowed to warm up to r.t. with monitoring of stirring rate. After stirring at r.t. for 1 h, the reaction mixture was treated with 1 M HCl solution (73 mL) and stirred at r.t. for 1 h. To the reaction mixture was added ammonium hydroxide (117 mL), followed by extraction with ethyl acetate. The organic phases were combined and washed with brine, dried over Na₂SO₄ and concentrated in vacuo. The crude material was added to iced water. The precipitated solid was filtered and dried to give the desired product as pale yellow solid (8.66 g, 25.5 mmol, 83% yield). LCMS calculated for C₁₃H₁₆BrN₄O₂(M+H)⁺: m/z=339.0; found 339.0.

### Step 3. Ethyl (1S,2S)-1-(6-bromo-3-iodo-1H-pyrazolo[3,4-b]pyridin-1-yl)-2-methylcyclopropane-1-carboxylate

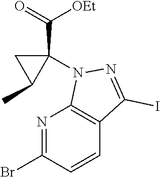 [SMILES: CCOC(=O)[C@]1(n2nc(I)c3ccc(Br)nc32)C[C@@H]1C]

To a solution of ethyl (1S,2S)-1-(3-amino-6-bromo-1H-pyrazolo[3,4-b]pyridin-1-yl)-2-methylcyclopropane-1-carboxylate (9 g, 25.5 mmol) in acetonitrile (128 mL) was added KI (4.2 g, 25.5 mmol) followed by I₂ (13 g, 51.1 mmol). To the obtained mixture was added tert-butyl nitrite (90%, 13.5 mL, 102 mmol) and the reaction mixture was stirred at r.t. for 20 h. The mixture was then quenched with sat. aq. Na₂S₂O₃ and aq. NaHCO₃, followed by extraction with EtOAc. The organic phases were combined and washed with brine, dried over Na₂SO₄ and concentrated in vacuo. The crude material was purified by Combi-Flash to give the desired product as pale yellow solid (7.7 g, 17.1 mmol, 67% yield). LCMS calculated for C₁₃H₁₄BrIN₃O₂ (M+H)⁺: m/z=449.9; found 450.1.

### Step 4. Ethyl (1S,2S)-1-(6-bromo-3-ethoxy-1H-pyrazolo[3,4-b]pyridin-1-yl)-2-methylcyclopropane-1-carboxylate

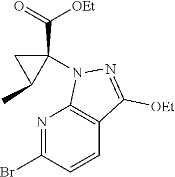 [SMILES: CCOC(=O)[C@]1(n2nc(OCC)c3ccc(Br)nc32)C[C@@H]1C]

A mixture of ethyl (1S,2S)-1-(6-bromo-3-iodo-1H-pyrazolo[3,4-b]pyridin-1-yl)-2-methylcyclopropane-1-carboxylate (500 mg, 1.11 mmol), copper(I) iodide (42 mg, 0.222 mmol), 3,4,7,8-tetramethyl-1,10-phenanthroline (105 mg, 0.444 mmol), and Cs₂CO₃ (471 mg, 1.444 mmol) in EtOH (10 mL) was irradiated in a microwave vial at 75° C. for 1.5 h. The reaction mixture was cooled to r.t., filtered through celite, washed with MeOH, and concentrated. The crude was purified by Combi-Flash to obtain the desired product. LCMS calculated for C₁₅H₁₉BrN₃O₃(M+H)⁺: m/z=368.1; found 368.2.

### Step 5. (1S,2S)-1-(6-Bromo-3-ethoxy-1H-pyrazolo[3,4-b]pyridin-1-yl)-2-methylcyclopropane-1-carboxylic acid

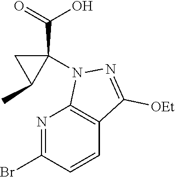 [SMILES: CCOc1nn([C@@]2(C(=O)O)C[C@@H]2C)c2nc(Br)ccc12]

To a mixture of ethyl (1S,2S)-1-(6-bromo-3-ethoxy-1H-pyrazolo[3,4-b]pyridin-1-yl)-2-methylcyclopropane-1-carboxylate (1.30 g, 3.53 mmol) in THF (7 mL), MeOH (7 mL) and water (7 mL) was added lithium hydroxide (0.85 g, 35.3 mmol), and the reaction mixture was heated to 50° C. for 3 h. The reaction was then cooled to r.t. and quenched with aq. HCl (1M, 40 mL, 40 mmol). The mixture was diluted with EtOAc and the organic layer was extracted, dried over Na₂SO₄, filtered and concentrated. The obtained product was used in the next step. LCMS calculated for C₁₃H₁₅BrN₃O₃(M+H)⁺: m/z=340.0; found 340.0.

### Step 6. (1S,2S)-1-(6-Bromo-3-ethoxy-1H-pyrazolo[3,4-b]pyridin-1-yl)-2-methylcyclopropane-1-carboxamide

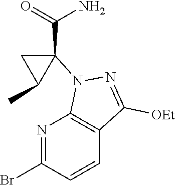 [SMILES: CCOc1nn([C@@]2(C(N)=O)C[C@@H]2C)c2nc(Br)ccc12]

A solution of (1S,2S)-1-(6-bromo-3-ethoxy-1H-pyrazolo[3,4-b]pyridin-1-yl)-2-methylcyclopropane-1-carboxylic acid (1.20 g, 3.53 mmol), HATU (2.01 g, 5.29 mmol), N,N-diisopropylethylamine (1.85 mL, 10.58 mmol), and ammonia (0.4 M in dioxane, 35 mL, 14.1 mmol) in DMF (18 mL) was stirred at 50° C. for 1.5 h. The reaction mixture was cooled to r.t., and the solvent was removed. The obtained material was diluted with DCM and water. The organic layer was extracted, washed with brine, dried over Na₂SO₄, filtered and concentrated. The crude was purified by Combi-Flash to obtain desired product as white foam (1.14 g, 3.36 mmol, 95% yield). LCMS calculated for C₁₃H₁₆BrN₄O₂(M+H)⁺: m/z=339.0; found 339.1.

### Step 7. Methyl-d3 ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate

A mixture of methyl-d₃ ((1R,3R)-3-(6-amino-3-(methyl-d₃)-2-oxo-2,3-dihydro-1H-imidazo[4,5-c]pyridin-1-yl)cyclopentyl)carbamate (597 mg, 1.92 mmol), (1S,2S)-1-(6-bromo-3-ethoxy-1H-pyrazolo[3,4-b]pyridin-1-yl)-2-methylcyclopropane-1-carboxamide (650 mg, 1.916 mmol), rac-BINAP-Pd-G3 (285 mg, 0.287 mmol), and cesium carbonate (1.25 g, 3.83 mmol) in dioxane (14 mL) was heated at 100° C. for 20 h. The reaction was then cooled to r.t. and filtered through celite, washed with MeOH. The obtained solution was stirred with SiliaMetS thiol metal scavenger for 30 min, then filtered and concentrated. The obtained material was taken up in EtOAc and filtered. The filtrate containing desired product was concentrated. The crude was purified by Combi-Flash (EtOAc/Hex then MeOH/DCM), followed by prep-LCMS (XBridge C18 column, acetonitrile/water containing 0.10% TFA, at flow rate of 60 mL/min). LCMS calculated for C₂₇H₂₈D₆N₉O₅(M+H)⁺: m/z=570.3; found 570.3. ¹H NMR (600 MHz, DMSO-d₆) δ 11.06 (s, 1H), 8.23 (s, 1H), 8.01 (d, J=8.5 Hz, 1H), 7.84 (s, 1H), 7.36-7.29 (m, 2H), 6.91 (d, J=8.6 Hz, 1H), 6.76 (s, 1H), 4.95-4.87 (m, 1H), 4.49-4.30 (m, 2H), 4.28-4.21 (m, 1H), 2.42-2.32 (m, 1H), 2.26-2.02 (m, 4H), 1.99-1.91 (m, 1H), 1.79-1.61 (m, 2H), 1.49-1.44 (m, 1H), 1.41 (t, J=7.0 Hz, 3H), 1.32 (d, J=6.3 Hz, 3H) ppm.

### Example 94. Methyl-d₃ ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

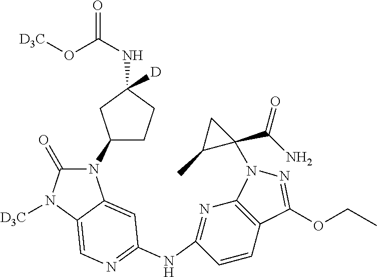 [SMILES: [2H]C([2H])([2H])OC(=O)N[C@]1([2H])CC[C@@H](n2c(=O)n(C([2H])([2H])[2H])c3cnc(Nc4ccc5c(OCC)nn([C@@]6(C(N)=O)C[C@@H]6C)c5n4)cc32)C1]

A mixture of methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate (20 mg, 0.035 mmol) and cesium carbonate (46 mg, 0.14 mmol) in CD₃OD (1 mL; D %≥99.8%) in a microwave vial was irradiated at 120° C. for 2 h. The reaction was cooled to r.t., quenched with TFA, and purified by prep-LCMS (XBridge C18 column, acetonitrile/water containing 0.1% TFA, at flow rate of 60 mL/min). LCMS calculated for C₂₇H₂₇D₇N₉O₅(M+H)⁺: m/z=571.3; found 571.4. ¹H NMR (500 MHz, DMSO-d₆) δ 10.90 (s, 1H), 8.20 (s, 1H), 7.99 (d, J=8.7 Hz, 1H), 7.91 (s, 1H), 7.39-7.22 (m, 2H), 6.93 (d, J=8.7 Hz, 1H), 6.72 (s, 1H), 4.99-4.83 (m, 1H), 4.45-4.32 (m, 2H), 2.40-2.34 (m, 1H), 2.26-2.00 (m, 4H), 1.99-1.89 (m, 1H), 1.78-1.72 (m, 1H), 1.69-1.61 (m, 1H), 1.50-1.44 (m, 1H), 1.41 (t, J=7.0 Hz, 3H), 1.32 (d, J=6.3 Hz, 3H) ppm.

### Example 95. Methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate

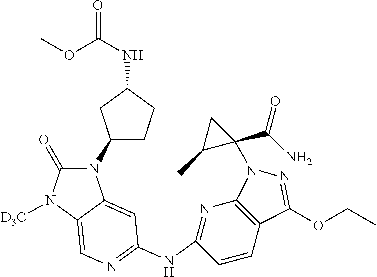 [SMILES: [2H]C([2H])([2H])n1c(=O)n([C@@H]2CC[C@@H](NC(=O)OC)C2)c2cc(Nc3ccc4c(OCC)nn([C@@]5(C(N)=O)C[C@@H]5C)c4n3)ncc21]

### Step 1. Methyl ((1R,3R)-3-(6-amino-3-(methyl-d₃)-2-oxo-2,3-dihydro-1H-imidazo[4,5-c]pyridin-1-yl)cyclopentyl)carbamate

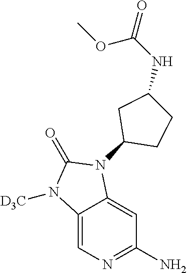 [SMILES: [2H]C([2H])([2H])n1c(=O)n([C@@H]2CC[C@@H](NC(=O)OC)C2)c2cc(N)ncc21]

This compound was prepared according to the procedures described in the synthesis of Intermediate C, with methyl carbonochloridate replacing methyl-d₃ carbonochloridate. LCMS calculated for C₁₄H₁₇D₃N₅O₃(M+H)⁺: m/z=309.2; found: 309.1.

### Step 2. Methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate

This compound was prepared according to the procedures described in Example 93, with methyl ((1R,3R)-3-(6-amino-3-(methyl-d₃)-2-oxo-2,3-dihydro-1H-imidazo[4,5-c]pyridin-1-yl)cyclopentyl)carbamate replacing methyl-d₃ ((1R,3R)-3-(6-amino-3-(methyl-d₃)-2-oxo-2,3-dihydro-1H-imidazo[4,5-c]pyridin-1-yl)cyclopentyl)carbamate. LCMS calculated for C₂₇H₃₁D₃N₉O₅(M+H)⁺: m/z=567.3; found: 567.4.

### Example 96. Methyl-d₃ ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate

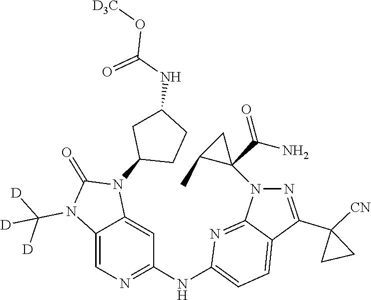 [SMILES: [2H]C([2H])([2H])OC(=O)N[C@@H]1CC[C@@H](n2c(=O)n(C([2H])([2H])[2H])c3cnc(Nc4ccc5c(C6(C#N)CC6)nn([C@@]6(C(N)=O)C[C@@H]6C)c5n4)cc32)C1]

### Step 1. Ethyl (1S,2S)-1-(3-amino-6-chloro-1H-pyrazolo[3,4-b]pyridin-1-yl)-2-methylcyclopropane-1-carboxylate

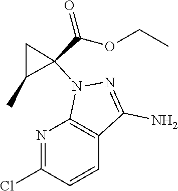 [SMILES: CCOC(=O)[C@]1(n2nc(N)c3ccc(Cl)nc32)C[C@@H]1C]

The title compound was prepared according to the procedures described in Example 54, Step 5-6, with ethyl (1S,2S)-1-amino-2-methylcyclopropane-1-carboxylate replacing benzyl (1S,2S)-1-amino-2-methylcyclopropane-1-carboxylate and 6-chloro-2-fluoronicotinonitrile replacing 6-bromo-2-fluoronicotinonitrile. LCMS calculated for C₁₃H₁₆ClN₄O₂(M+H)⁺: m/z=295.1; found 295.1.

### Step 2. Ethyl (1S,2S)-1-(6-chloro-3-iodo-1H-pyrazolo[3,4-b]pyridin-1-yl)-2-methylcyclopropane-1-carboxylate

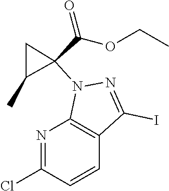 [SMILES: CCOC(=O)[C@]1(n2nc(I)c3ccc(Cl)nc32)C[C@@H]1C]

To a vial containing ethyl (1S,2S)-1-(3-amino-6-chloro-1H-pyrazolo[3,4-b]pyridin-1-yl)-2-methylcyclopropane-1-carboxylate (11.7 g, 39.7 mmol) in MeCN (200 mL) was added potassium iodide (6.6 g, 39.7 mmol) and iodine (20.1 g, 79 mmol). The reaction mixture was cooled to 0° C. before tert-butyl nitrite (18.9 mL, 159 mmol) was added in one portion and was heated to 60° C. for 2 h. After cooling to r.t., the mixture was quenched with sat. Na₂S₂O₃ aq. solution followed by extraction with DCM. The organic phases were combined and washed with brine, dried over MgSO₄ and concentrated in vacuo. The crude material was purified by Biotage Isolera to give the desired product. LCMS calculated for C₁₃H₁₄ClIN₃O₂ (M+H)⁺: m/z=406.0; found 406.1.

### Step 3. Ethyl (1S,2S)-1-(6-chloro-3-formyl-1H-pyrazolo[3,4-b]pyridin-1-yl)-2-methylcyclopropane-1-carboxylate

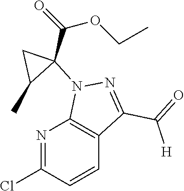 [SMILES: CCOC(=O)[C@]1(n2nc(C=O)c3ccc(Cl)nc32)C[C@@H]1C]

The title compound was prepared according to the procedures described in Example 17, Step 3-4, with ethyl (1S,2S)-1-(6-chloro-3-iodo-1H-pyrazolo[3,4-b]pyridin-1-yl)-2-methylcyclopropane-1-carboxylate replacing ethyl 1-(6-bromo-3-iodo-1H-pyrazolo[3,4-b]pyridin-1-yl)cyclobutane-1-carboxylate. LCMS calculated for C₁₄H₁₅ClN₃O₃(M+H)⁺: m/z=308.1; found 308.1.

### Step 4. Ethyl (1S,2S)-1-(6-chloro-3-(hydroxymethyl)-1H-pyrazolo[3,4-b]pyridin-1-yl)-2-methylcyclopropane-1-carboxylate

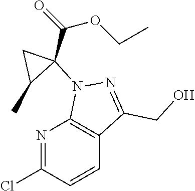 [SMILES: CCOC(=O)[C@]1(n2nc(CO)c3ccc(Cl)nc32)C[C@@H]1C]

To a cooled flask containing ethyl (1S,2S)-1-(6-chloro-3-formyl-1H-pyrazolo[3,4-b]pyridin-1-yl)-2-methylcyclopropane-1-carboxylate (1.17 g, 30.8 mmol) in THF (30 mL) and MeOH (6 mL) was added sodium borohydride (6.58 g, 39.7 mmol) portionwise. The mixture was stirred at 0° C. for 20 min. before quenched with water, followed by extraction with EtOAc. The organic phases were combined and washed with brine, dried over MgSO₄ and concentrated in vacuo. The crude material was purified by Biotage Isolera to give the desired product. LCMS calculated for C₁₄H₁₇ClN₃O₃(M+H)⁺: m/z=310.1; found 310.1.

### Step 5. Ethyl (1S,2S)-1-(6-chloro-3-(cyanomethyl)-1H-pyrazolo[3,4-b]pyridin-1-yl)-2-methylcyclopropane-1-carboxylate

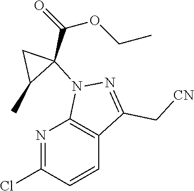 [SMILES: CCOC(=O)[C@]1(n2nc(CC#N)c3ccc(Cl)nc32)C[C@@H]1C]

To a cooled flask containing ethyl (1S,2S)-1-(6-chloro-3-(hydroxymethyl)-1H-pyrazolo[3,4-b]pyridin-1-yl)-2-methylcyclopropane-1-carboxylate (1.78 g, 5.75 mmol) and triethylamine (1.6 mL, 11.5 mmol) in DCM (30 mL) was added methanesulfonyl chloride (0.53 mL, 6.90 mmol) dropwise. The mixture was stirred at 0° C. for 20 min. before quenched with water followed by extraction with DCM. The organic phases were combined and washed with brine, dried over MgSO₄ and concentrated in vacuo. To the crude material was added NaCN (845 mg, 17.25 mmol) and DMF (20 mL) and the mixture was stirred at r.t. for 3 h before quenched with water followed by extraction with EtOAc. The organic phases were combined and washed with brine, dried over MgSO₄ and concentrated in vacuo. The crude material was purified by Biotage Isolera to give the desired product. LCMS calculated for C₁₅H₁₆ClN₄O₂(M+H)⁺: m/z=319.1; found 319.1.

### Step 6. Ethyl (1S,2S)-1-(6-chloro-3-(1-cyanocyclopropyl)-1H-pyrazolo[3,4-b]pyridin-1-yl)-2-methylcyclopropane-1-carboxylate

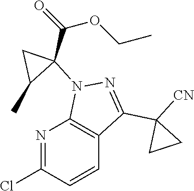 [SMILES: CCOC(=O)[C@]1(n2nc(C3(C#N)CC3)c3ccc(Cl)nc32)C[C@@H]1C]

To a flask containing ethyl (1S,2S)-1-(6-chloro-3-(cyanomethyl)-1H-pyrazolo[3,4-b]pyridin-1-yl)-2-methylcyclopropane-1-carboxylate (960 mg, 3.01 mmol) in THF (20 mL) at −78° C. was slowly added LHMDS (3.61 mL, 3.61 mmol, 1.0 M in THF). The mixture was stirred at −78° C. for 10 min. before a solution of 1,3,2-dioxathiolane 2,2-dioxide (449 mg, 3.61 mmol) in THF (2 mL) was added dropwise. The resulting mixture was stirred at 0° C. for 1 h before it was cooled to −78° C. and LHMDS (3.61 mL, 3.61 mmol, 1.0 M in THF) was added slowly. The mixture was stirred at 0° C. for 1 h before it was quenched with water followed by extraction with EtOAc. The organic phases were combined and washed with brine, dried over MgSO₄ and concentrated in vacuo. The crude material was purified by Biotage Isolera to give the desired product. LCMS calculated for C₁₇H₁₈ClN₄O₂(M+H)⁺: m/z=345.1; found 345.1.

### Step 6. Methyl-d3 ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate

This compound was prepared according to the procedure in Example 14, Steps 3-4, with methyl-d₃ ((1R,3R)-3-(6-amino-3-(methyl-d₃)-2-oxo-2,3-dihydro-1H-imidazo[4,5-c]pyridin-1-yl)cyclopentyl)carbamate (Intermediate C) replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate and ethyl (1S,2S)-1-(6-chloro-3-(1-cyanocyclopropyl)-1H-pyrazolo[3,4-b]pyridin-1-yl)-2-methylcyclopropane-1-carboxylate replacing methyl 1-(6-bromo-1H-pyrazolo[3,4-b]pyridin-1-yl)-3,3-difluorocyclobutane-1-carboxylate in Step 3. LCMS calculated for: C₂₉H₂₇D₆N₁₀O₄(M+H)⁺: m/z=591.3; found 591.3.

### Example 97. Methyl-d₃ ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

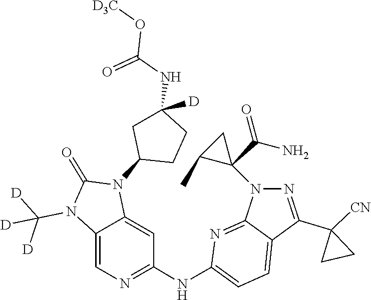 [SMILES: [2H]C([2H])([2H])OC(=O)N[C@]1([2H])CC[C@@H](n2c(=O)n(C([2H])([2H])[2H])c3cnc(Nc4ccc5c(C6(C#N)CC6)nn([C@@]6(C(N)=O)C[C@@H]6C)c5n4)cc32)C1]

This compound was prepared according to the procedure in Example 96, Steps 1-6, with methyl-d₃ ((1R,3R)-3-(6-amino-3-(methyl-d₃)-2-oxo-2,3-dihydro-1H-imidazo[4,5-c]pyridin-1-yl)cyclopentyl-1-d)carbamate (Example 90, Step 1) replacing methyl-d₃ ((1R,3R)-3-(6-amino-3-(methyl-d₃)-2-oxo-2,3-dihydro-1H-imidazo[4,5-c]pyridin-1-yl)cyclopentyl)carbamate (Intermediate C) in Step 6. LCMS calculated for: C₂₉H₂₆D₇N₁₀O₄ (M+H)⁺: m/z=592.3; found 592.3. ¹H NMR (500 MHz, DMSO) δ 10.96 (s, 1H), 8.25-8.19 (m, 2H), 7.92 (s, 1H), 7.37 (s, 1H), 7.29 (s, 1H), 7.10 (d, Js=9.0 Hz, 1H), 6.85 (s, 1H), 4.97-4.89 (m, 1H), 2.39 (dd, J=13.5, 7.4 Hz, 1H), 2.26-2.01 (m, 4H), 1.95 (dd, J=13.6, 9.5 Hz, 1H), 1.85-1.75 (m, 5H), 1.68-1.62 (m, 1H), 1.43 (dd, J=9.8, 4.9 Hz, 1H), 1.33 (d, J=6.3 Hz, 3H).

### Example 98. Methyl ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

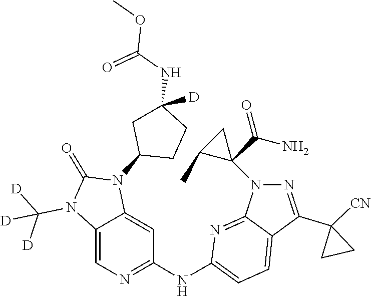 [SMILES: [2H]C([2H])([2H])n1c(=O)n([C@@H]2CC[C@@]([2H])(NC(=O)OC)C2)c2cc(Nc3ccc4c(C5(C#N)CC5)nn([C@@]5(C(N)=O)C[C@@H]5C)c4n3)ncc21]

This compound was prepared according to the procedure in Example 96, Steps 1-6, with methyl ((1R,3R)-3-(6-amino-3-(methyl-d₃)-2-oxo-2,3-dihydro-1H-imidazo[4,5-c]pyridin-1-yl)cyclopentyl-1-d)carbamate (Intermediate A) replacing methyl-d₃ ((1R,3R)-3-(6-amino-3-(methyl-d₃)-2-oxo-2,3-dihydro-1H-imidazo[4,5-c]pyridin-1-yl)cyclopentyl)carbamate (Intermediate C) in Step 6. LCMS calculated for: C₂₉H₂₉D₄N₁₀O₄ (M+H)⁺: m/z=589.3; found 589.3. ¹H NMR (500 MHz, DMSO) δ 10.89 (s, 1H), 8.24-8.18 (m, 2H), 7.94 (s, 1H), 7.37 (s, 1H), 7.29 (s, 1H), 7.10 (d, J=8.9 Hz, 1H), 6.84 (s, 1H), 4.97-4.89 (m, 1H), 3.54 (s, 3H), 2.39 (dd, J=13.5, 7.4 Hz, 1H), 2.26-2.01 (m, 4H), 1.95 (dd, J=13.5, 9.5 Hz, 1H), 1.87-1.73 (m, 5H), 1.68-1.62 (m, 1H), 1.43 (dd, J=9.8, 4.8 Hz, 1H), 1.33 (d, J=6.3 Hz, 3H).

### Example 99. Methyl ((1R,3R)-3-(6-((1-((1S,2S)-1-carbamoyl-2-(difluoromethyl)cyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

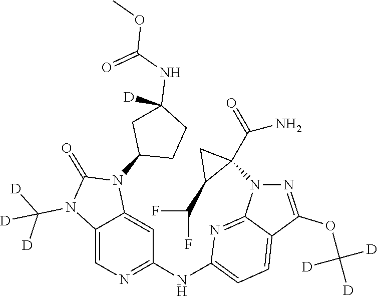 [SMILES: [2H]C([2H])([2H])Oc1nn([C@@]2(C(N)=O)C[C@@H]2C(F)F)c2nc(Nc3cc4c(cn3)n(C([2H])([2H])[2H])c(=O)n4[C@@H]3CC[C@@]([2H])(NC(=O)OC)C3)ccc12]

The title compound was prepared according to the procedures described in Example 53 with methyl (1S,2S)-1-amino-2-(difluoromethyl)cyclopropane-1-carboxylate (Example 42, Step 2) replacing benzyl (1S,2S)-1-amino-2-methylcyclopropane-1-carboxylate in step 5, and methanol-d₃ (Sigma Adrich ≥99.8% D) replacing ethanol in step 10. LCMS calculated for C₂₆H₂₃D₇F₂N₉O₅ (M+H)⁺: m/z=593.3; found 593.3. ¹H NMR (500 MHz, DMSO) δ 10.83 (s, 1H), 8.16 (s, 1H), 8.00 (d, J=8.6 Hz, 1H), 7.89 (s, 1H), 7.63 (s, 1H), 7.27 (s, 1H), 7.10 (s, 1H), 6.99 (d, J=8.7 Hz, 1H), 6.43-6.11 (m, 1H), 4.90-4.81 (m, 1H), 3.55 (s, 3H), 2.51 (m, 1H), 2.37 (m, 1H), 2.24-2.10 (m, 3H), 2.04 (m, 1H), 1.95 (m, 2H), 1.66 (m, 1H). ¹⁹F NMR (471 MHz, DMSO) δ −100.97-−115.78 (m).

### Example 100. Methyl ((1R,3R)-3-(6-((7-((1R,2S)-1-carbamoyl-2-methylcyclopropyl)-4-methyl-5-(methyl-d₃)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

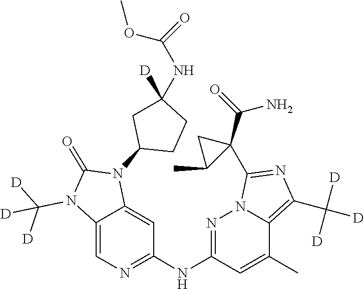 [SMILES: [2H]C([2H])([2H])c1nc([C@@]2(C(N)=O)C[C@@H]2C)n2nc(Nc3cc4c(cn3)n(C([2H])([2H])[2H])c(=O)n4[C@@H]3CC[C@@]([2H])(NC(=O)OC)C3)cc(C)c12]

### Step 1. Ethyl (1R,2S)-1-(2-chloroimidazo[1,5-b]pyridazin-7-yl)-2-methylcyclopropane-1-carboxylate

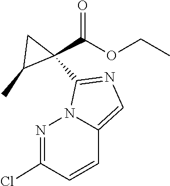 [SMILES: CCOC(=O)[C@]1(c2ncc3ccc(Cl)nn23)C[C@@H]1C]

The title compound was prepared according to the procedures described in Example 5, Step 1-2, with (1R,2S)-1-((ethoxy)carbonyl)-2-methylcyclopropane-1-carboxylic acid [Birdotech, cat #AC16434], replacing 3-fluoro-1-(isopropoxycarbonyl)cyclobutane-1-carboxylic acid in Step 1. The crude product was purified by flash column chromatography on silica (0-40% EtOAc in hexanes) to afford the title compound (5.05 g, 18.0 mmol, 67% yield). LCMS calculated for C₁₃H₁₅ClN₃O₂(M+H)⁺: m/z=280.1; found 280.1. ¹H NMR (400 MHz, CDCl₃) δ 7.71 (d, J=9.4 Hz, 1H), 7.41 (s, 1H), 6.55 (d, J=9.4 Hz, 1H), 4.21-4.01 (m, 2H), 2.31-2.18 (m, 1H), 1.74 (dd, J=8.0, 4.6 Hz, 1H), 1.40 (d, J=6.4 Hz, 3H), 1.34 (dd, J=9.1, 4.6 Hz, 1H), 1.10 (t, J=7.5 Hz, 3H).

### Step 2. Ethyl (1R,2S)-1-(2-chloro-4-iodoimidazo[1,5-b]pyridazin-7-yl)-2-methylcyclopropane-1-carboxylate

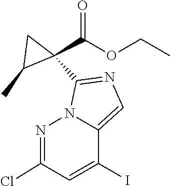 [SMILES: CCOC(=O)[C@]1(c2ncc3c(I)cc(Cl)nn23)C[C@@H]1C]

To a solution of ethyl (1R,2S)-1-(2-chloroimidazo[1,5-b]pyridazin-7-yl)-2-methylcyclopropane-1-carboxylate (226 mg, 0.81 mmol) in THF (1 mL) was added TMPZnCl·LiCl in THF/hexane (3.05 mL, 1.62 mmol) at r.t. and stirred for 18 h. To the reaction was added a solution of I₂ (411 mg, 1.62 mmol) in THF (1 mL) and stirred for 10 min at r.t. The reaction was quenched with saturated aqueous solution of Na₂S₂O₃ (3 mL) and extracted with EtOAc (3×1 mL). The combined organic layers were dried over MgSO₄, filtered, and concentrated under reduced pressure. The residue was purified by flash column chromatography on silica gel. (0-40% EtOAc in hexanes) to give the title compound (291 mg, 0.72 mmol, 89% yield). LCMS calculated for C₁₃H₁₄ClIN₃O₂(M+H)⁺: m/z=405.9; found 405.9. ¹H NMR (400 MHz, CDCl₃) δ 7.40 (s, 1H), 7.09 (s, 1H), 4.23-4.03 (m, 2H), 2.32-2.18 (m, 1H), 1.76 (dd, J=8.1, 4.6 Hz, 1H), 1.41 (d, J=6.3 Hz, 3H), 1.37 (dd, J=9.1, 4.6 Hz, 1H), 1.12 (t, J=7.1 Hz, 3H).

### Step 3. Ethyl (1R,2S)-1-(2-chloro-4-methylimidazo[1,5-b]pyridazin-7-yl)-2-methylcyclopropane-1-carboxylate

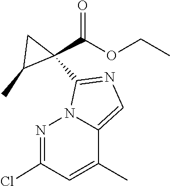 [SMILES: CCOC(=O)[C@]1(c2ncc3c(C)cc(Cl)nn23)C[C@@H]1C]

A screw-cap vial equipped with a magnetic stir bar was charged ethyl (1R,2S)-1-(2-chloro-4-iodoimidazo[1,5-b]pyridazin-7-yl)-2-methylcyclopropane-1-carboxylate (290 mg, 0.71 mmol), PdCl₂(dppf) (209 mg, 0.29 mmol), Cs₂CO₃ (1165 mg, 3.57 mmol), trimethylboroxine (300 μL, 1.24 mmol), 1,4-dioxane (5.7 mL), and water (1.4 mL). The mixture was stirred at 80° C. for 3 h. The reaction was cooled down to r.t. and extracted with EtOAc (3×5 mL). The combined organic layers were dried over MgSO₄, filtered, and concentrated under reduced pressure. The residue was purified by flash column chromatography on silica gel. (0-40% EtOAc in hexanes) to give the title compound (180 mg, 0.61 mmol, 86% yield). LCMS calculated for Cl₄H₁₇ClN₃O₂(M+H)⁺: m/z=294.1; found 294.0.

### Step 4. Ethyl (1R,2S)-1-(2-chloro-5-iodo-4-methylimidazo[1,5-b]pyridazin-7-yl)-2-methylcyclopropane-1-carboxylate

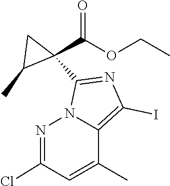 [SMILES: CCOC(=O)[C@]1(c2nc(I)c3c(C)cc(Cl)nn23)C[C@@H]1C]

The title compound was prepared according to the procedures described in Example 6, Step 1, with ethyl (1R,2S)-1-(2-chloro-4-methylimidazo[1,5-b]pyridazin-7-yl)-2-methylcyclopropane-1-carboxylate, replacing isopropyl 1-(2-chloroimidazo[1,5-b]pyridazin-7-yl)-3-fluorocyclobutane-1-carboxylate. The crude product was purified by flash column chromatography on silica (0-40% EtOAc in hexanes) to afford the title compound (135 mg, 0.32 mmol, 52% yield). LCMS calculated for C₁₄H₁₆ClIN₃O₂(M+H)⁺: m/z=420.0; found 419.9.

### Step 5. Ethyl (1R,2S)-1-(2-chloro-4-methyl-5-(methyl-d₃)imidazo[1,5-b]pyridazin-7-yl)-2-methylcyclopropane-1-carboxylate

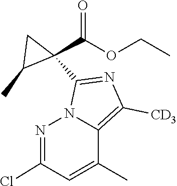 [SMILES: [2H]C([2H])([2H])c1nc([C@@]2(C(=O)OCC)C[C@@H]2C)n2nc(Cl)cc(C)c12]

The title compound was prepared according to the procedures described in Example 54, Step 10, with ethyl (1R,2S)-1-(2-chloro-5-iodo-4-methylimidazo[1,5-b]pyridazin-7-yl)-2-methylcyclopropane-1-carboxylate and (methyl-d₃)zinc(II) chloride, replacing benzyl (1S,2S)-1-(3-iodo-6-((1-((1R,3R)-3-((methoxycarbonyl)amino)-cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylate and methylzinc chloride, respectively. The crude material was purified by flash column chromatography on silica (0-40% EtOAc in hexanes) to afford the title compound (25 mg, 0.08 mmol, 25% yield). LCMS calculated for C₁₅H₁₆D₃ClN₃O₂ (M+H)⁺: m/z=311.1; found 311.1.

### Step 6. Methyl ((1R,3R)-3-(6-((7-((1R,2S)-1-carbamoyl-2-methylcyclopropyl)-4-methyl-5-(methyl-d₃)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example 59, Step 4, replacing benzyl (1R,2S)-1-(2-chloro-5-(methyl-d₃)imidazo[1,5-b]pyridazin-7-yl)-2-methylcyclopropane-1-carboxylate with ethyl (1R,2S)-1-(2-chloro-4-methyl-5-(methyl-d₃)imidazo[1,5-b]pyridazin-7-yl)-2-methylcyclopropane-1-carboxylate. The final crude mixture was purified by flash column chromatography on silica (0-40% MeOH in CH₂Cl₂), then further purified using prep-LCMS (XBridge C18 column, eluting with a gradient of acetonitrile/water containing 0.1% NH₄OH, at flow rate of 60 mL/min) to give the title compound. LCMS calculated for: C₂₇H₂₇D₇N₉O₄(M+H)⁺: m/z=555.3; found 555.4. ¹H NMR (600 MHz, DMSO-d₆) δ 9.67 (s, 1H), 8.14 (s, 1H), 8.06 (s, 1H), 7.20 (s, 1H), 7.10 (s, 1H), 6.63 (s, 1H), 6.40 (s, 1H), 5.20-5.12 (m, 1H), 3.50 (s, 3H), 2.48-2.43 (m, 1H), 2.44 (s, 3H), 2.22-2.16 (m, 1H), 2.16-2.08 (m, 1H), 2.08-2.01 (m, 1H), 2.01-1.93 (m, 2H), 1.66-1.58 (m, 1H), 1.52 (dd, J=7.4, 4.0 Hz, 1H), 1.27 (d, J=6.4 Hz, 3H), 0.99 (dd, J=8.8, 4.0 Hz, 1H).

### Example 101. Methyl ((1R,3R)-3-(6-((7-((1R,2S)-1-carbamoyl-2-methylcyclopropyl)-4-methyl-5-(methyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

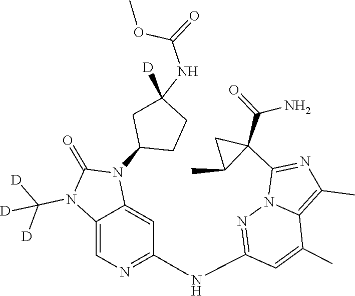 [SMILES: [2H]C([2H])([2H])n1c(=O)n([C@@H]2CC[C@@]([2H])(NC(=O)OC)C2)c2cc(Nc3cc(C)c4c(C)nc([C@@]5(C(N)=O)C[C@@H]5C)n4n3)ncc21]

The title compound was prepared according to the procedures described in Example 100, Step 1-6, replacing (methyl-d₃)zinc(II) chloride with methylzinc chloride in Step 5. The final crude material was purified by flash column chromatography on silica (0-40% EtOAc in hexanes) then further purified using prep-LCMS (XBridge C18 column, eluting with a gradient of acetonitrile/water containing 0.1% NH₄OH, at flow rate of 60 mL/min) to give the title compound. LCMS calculated for: C₂₇H₃₀D₄N₉O₄ (M+H)⁺: m/z=552.3; found 552.3. ¹H NMR (400 MHz, 1:1 mixture of CDCl₃ and MeOD) δ 8.26 (s, 1H), 7.91 (d, J=4.1 Hz, 1H), 7.67-7.56 (m, 4H), 6.23 (s, 1H), 5.21-5.05 (m, 1H), 3.59 (s, 3H), 2.69-2.60 (m, 1H), 2.58 (s, 3H), 2.52 (s, 3H), 2.42-2.20 (m, 3H), 2.03-1.88 (m, 2H), 1.84-1.74 (m, 1H), 1.73-1.66 (m, 1H), 1.36 (d, J=6.2 Hz, 3H), 1.21 (dd, J=8.9, 4.5 Hz, 1H).

### Example 102. Methyl-d₃ (4-(6-((1-((1s,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)-2-fluorocyclopentyl)carbamate

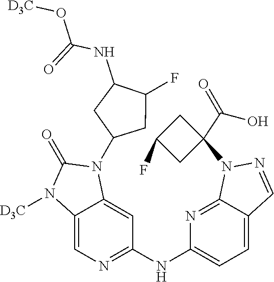 [SMILES: [2H]C([2H])([2H])OC(=O)NC1CC(n2c(=O)n(C([2H])([2H])[2H])c3cnc(Nc4ccc5cnn([C@]6(C(=O)O)C[C@H](F)C6)c5n4)cc32)CC1F]

### Step 1. Isopropyl (1s,3R)-3-fluoro-1-(6-((1-(3-fluoro-4-((methoxycarbonyl)amino)cyclopentyl)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

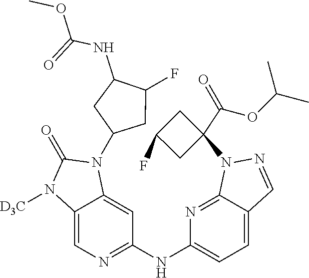 [SMILES: [2H]C([2H])([2H])n1c(=O)n(C2CC(F)C(NC(=O)OC)C2)c2cc(Nc3ccc4cnn([C@]5(C(=O)OC(C)C)C[C@H](F)C5)c4n3)ncc21]

A vial containing methyl (4-(6-amino-3-(methyl-d₃)-2-oxo-2,3-dihydro-1H-imidazo[4,5-c]pyridin-1-yl)-2-fluorocyclopentyl)carbamate (36 mg, 0.10 mmol, Example 103, Step 6), isopropyl (1s,3s)-1-(6-amino-1H-pyrazolo[3,4-b]pyridin-1-yl)-3-fluorocyclobutane-1-carboxylate (30 mg, 0.10 mmol), rac-BINAP-Pd-G3 (10 mg, 10 mol), and cesium carbonate (67 mg, 0.21 mmol) was evacuated and backfilled with nitrogen three times, followed by the addition of 1,4-dioxane (0.5 mL). The vial was sealed and heated to 100° C. for 1 h. After cooling to r.t., the mixture was filtered and concentrated in vacuo. The crude material was used in the next step without further purification. LCMS calculated for C₂₈H₃₀D₃F₂N₈O₅(M+H)⁺: m/z=602.3; found 602.3.

### Step 2. (1s,3R)-3-Fluoro-1-(6-((1-(3-fluoro-4-(((methoxy-d₃)carbonyl)amino)cyclopentyl)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid

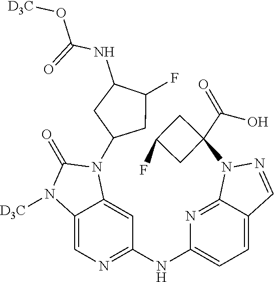 [SMILES: [2H]C([2H])([2H])OC(=O)NC1CC(n2c(=O)n(C([2H])([2H])[2H])c3cnc(Nc4ccc5cnn([C@]6(C(=O)O)C[C@H](F)C6)c5n4)cc32)CC1F]

To a microwave vial containing isopropyl (1s,3R)-3-fluoro-1-(6-((1-(3-fluoro-4-((methoxycarbonyl)amino)cyclopentyl)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (62 mg, 0.10 mmol) and cesium carbonate (67 mg, 0.21 mmol) was added methanol-d₄ (1.0 mL). The microwave vial was sealed and irradiated at 100° C. for 2 h. After cooling to r.t., the mixture was filtered and concentrated in vacuo. The obtained crude material was dissolved in THF/MeOH/water (1.0 mL, v/v/v=1/1/1) followed by addition of LiOH (24 mg, 1.0 mmol). After heating at 50° C. for 1 h, the reaction mixture was cooled to r.t. and diluted with CH₂Cl₂ (5 mL). The pH of the mixture was adjusted to ˜4 using HCl aqueous solution, followed by extraction with CH₂Cl₂ (3×5 mL). The organic layers were combined, dried over MgSO₄ and concentrated in vacuo. The crude material was used in the next step without further purification. LCMS calculated for C₂₅H₂₁D₆F₂N₈O₅ (M+H)⁺: m/z=563.2; found 563.2.

### Step 3. Methyl-d₃ (4-(6-((1-((1s,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)-2-fluorocyclopentyl)carbamate

The title compound was prepared according to the procedures described in Example 48, Step 5, with ((1s,3R)-3-fluoro-1-(6-((1-(3-fluoro-4-(((methoxy-d₃)carbonyl)amino)cyclopentyl)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid replacing (1R,2S)-2-(3-methoxy-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-[1,1′-bi(cyclopropane)]-2-carboxylic. LCMS calculated for C₂₅H₂₂D₆F₂N₉O₄ (M+H)⁺: m/z=562.3; found 562.3.

### Example 103. Methyl (4-(6-((1-((1s,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)-2-fluorocyclopentyl)carbamate

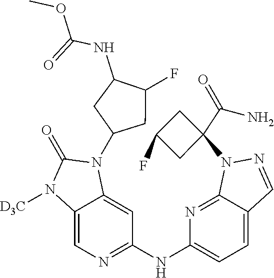 [SMILES: [2H]C([2H])([2H])n1c(=O)n(C2CC(F)C(NC(=O)OC)C2)c2cc(Nc3ccc4cnn([C@]5(C(N)=O)C[C@H](F)C5)c4n3)ncc21]

### Step 1. N-((1S,2S,4R)-2-Fluoro-4-hydroxycyclopentyl)-4-nitrobenzenesulfonamide and N-((1R,2R,4S)-2-fluoro-4-hydroxycyclopentyl)-4-nitrobenzenesulfonamide

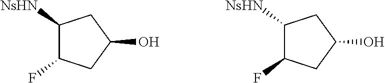

To a flask containing (1R,3r,5S)-6-((4-nitrophenyl)sulfonyl)-6-azabicyclo[3.1.0]hexan-3-ol (16.0 g, 56.3 mmol, Caine, D. et. al, Org. Lett. 2002, 4, 1923) in THF (100 mL) was added TBAF (84 mL, 84 mmol, 1.0 M in THF). The reaction was stirred at r.t. for 16 h and then concentrated in vacuo. The crude material was purified by Biotage Isolera to give the desired product as a racemic mixture. The racemic mixture was further separated by chiral SFC (CHIRALPAK IH, 30 mm×250 mm, 5 μm column, eluting with 25% IPA in CO₂, at flow rate of 90 mL/min over 10 min). The first peak was collected at 7.0 min as a single enantiomer. The second peak was collected at 8.0 min as a single enantiomer. Both peaks were individually carried forward through next steps. ¹H NMR (400 MHz, DMSO) δ 8.42 (d, J=8.7 Hz, 2H), 8.39 (s, 1H), 8.07 (d, J=8.7 Hz, 2H), 4.98-4.61 (m, 2H), 4.12-3.99 (m, 1H), 3.70-3.49 (m, 1H), 2.08 (dt, J=14.2, 7.4 Hz, 1H), 2.02-1.65 (m, 2H), 1.30-1.20 (m, 1H).

### Step 2. N-(4-(1,3-Dioxoisoindolin-2-yl)-2-fluorocyclopentyl)-4-nitrobenzenesulfonamide

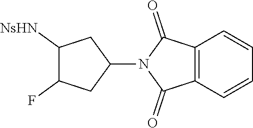

The title compound was prepared according to the procedures described in Intermediate A, Step 2, with N-(4-(1,3-dioxoisoindolin-2-yl)-2-fluorocyclopentyl)-4-nitrobenzenesulfonamide (single enantiomer, Peak 2 from Step 1) replacing tert-butyl ((1R,3S)-3-hydroxycyclopentyl-3-d)carbamate. LCMS calculated for C₁₉H₁₇FN₃O₆S (M+H)⁺: m/z=434.1; found: 434.2.

### Step 3. Methyl (4-(1,3-dioxoisoindolin-2-yl)-2-fluorocyclopentyl)carbamate

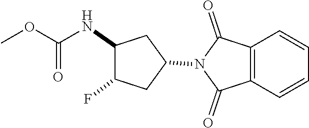 [SMILES: COC(=O)N[C@H]1C[C@H](N2C(=O)c3ccccc3C2=O)C[C@@H]1F]

To a vial containing rel-N-((1S,2S,4S)-4-(1,3-dioxoisoindolin-2-yl)-2-fluorocyclopentyl)-4-nitrobenzenesulfonamide (750 mg, 1.7 mmol) and potassium carbonate (0.96 g, 6.9 mmol) in DMF (3.5 mL) was added 4-methoxybenzenethiol (0.36 g, 0.32 mL, 2.6 mmol). After stirring at r.t. for 8 h, the reaction mixture was diluted with EtOAc (30 mL). The reaction mixture was washed with brine (3 mL) for 5 times, dried over MgSO₄, and concentrated in vacuo. The obtained crude material was dissolved in CH₂Cl₂ (20 mL) and MeOH (1 mL) followed by the addition of DIPEA (670 mg, 0.90 mL, 5.2 mmol). After cooling down to 0° C., methyl chloroformate (250 mg, 0.20 mL, 2.6 mmol) was added dropwise. After stirring at 0° C. for 10 min., the reaction was quenched with MeOH (5 mL) and concentrated in vacuo. The crude material was purified by Biotage Isolera to give the desired product as colorless oil. LCMS calculated for C₁₅H₁₆FN₂O₄(M+H)⁺: m/z=307.1; found: 307.1.

### Step 4. Methyl (4-amino-2-fluorocyclopentyl)carbamate

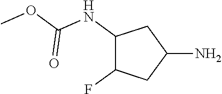

The title compound was prepared according to the procedures described in Intermediate A, Step 3, with methyl (4-(1,3-dioxoisoindolin-2-yl)-2-fluorocyclopentyl)carbamate replacing tert-butyl ((1R,3R)-3-(1,3-dioxoisoindolin-2-yl)cyclopentyl-3-d)carbamate. LCMS calculated for C₇H₁₄FN₂O₂(M+H)⁺: m/z=177.1; found: 177.1.

### Step 5. Methyl (4-(6-amino-3-(methyl-d₃)-2-oxo-2,3-dihydro-1H-imidazo[4,5-c]pyridin-1-yl)-2-fluorocyclopentyl)carbamate

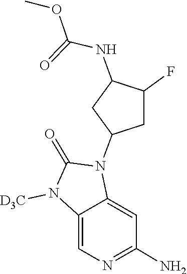 [SMILES: [2H]C([2H])([2H])n1c(=O)n(C2CC(F)C(NC(=O)OC)C2)c2cc(N)ncc21]

The title compound was prepared according to the procedures described in Intermediate A, Step 5 to 9, with methyl (4-amino-2-fluorocyclopentyl)carbamate replacing methyl ((1R,3R)-3-aminocyclopentyl-1-d)carbamate in Step 5. LCMS calculated for C₁₄H₁₆D₃FN₅O₃(M+H)⁺: m/z=327.2; found: 327.2.

### Step 7. (1s,3R)-3-fluoro-1-(6-((1-(3-fluoro-4-((methoxycarbonyl)amino)cyclopentyl)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid

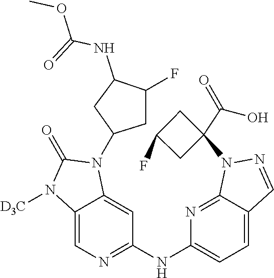 [SMILES: [2H]C([2H])([2H])n1c(=O)n(C2CC(F)C(NC(=O)OC)C2)c2cc(Nc3ccc4cnn([C@]5(C(=O)O)C[C@H](F)C5)c4n3)ncc21]

A vial containing methyl (4-(6-amino-3-(methyl-d₃)-2-oxo-2,3-dihydro-1H-imidazo[4,5-c]pyridin-1-yl)-2-fluorocyclopentyl)carbamate (36 mg, 0.10 mmol), isopropyl (1s,3s)-1-(6-amino-1H-pyrazolo[3,4-b]pyridin-1-yl)-3-fluorocyclobutane-1-carboxylate (30 mg, 0.10 mmol), rac-BINAP-Pd-G3 (10 mg, 10 mol), and cesium carbonate (67 mg, 0.21 mmol) was evacuated and backfilled with nitrogen three times, followed by the addition of 1,4-dioxane (0.5 mL). The vial was sealed and heated to 100° C. for 1 h. After cooling to r.t., the mixture was filtered and concentrated in vacuo. The obtained crude material was dissolved in THF/MeOH/water (1.0 mL, v/v/v=1/1/1) followed by addition of LiOH (24 mg, 1.0 mmol). After heating at 50° C. for 1 h, the reaction mixture was cooled to r.t. and diluted with CH₂Cl₂ (5 mL). The pH of the mixture was adjusted to ˜4 using HCl aqueous solution, followed by extraction with CH₂Cl₂ (3×5 mL). The organic layers were combined, dried over MgSO₄ and concentrated in vacuo. The crude material was used in the next step without further purification. LCMS calculated for C₂₅H₂₄D₃F₂N₈O₅ (M+H)⁺: m/z=560.2; found 560.2.

### Step 8. Methyl (4-(6-((1-((1s,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)-2-fluorocyclopentyl)carbamate

The title compound was prepared according to the procedures described in Example 48, Step 5, with (1s,3R)-3-fluoro-1-(6-((1-(3-fluoro-4-((methoxycarbonyl)amino)cyclopentyl)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid replacing (1R,2S)-2-(3-methoxy-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)-[1,1′-bi(cyclopropane)]-2-carboxylic acid. LCMS calculated for C₂₅H₂₅D₃F₂N₉O₄ (M+H)⁺: m/z=559.2; found 559.2. ¹H NMR (500 MHz, DMSO) δ 11.14 (brs, 1H), 8.25 (s, 1H), 8.20 (d, J=8.6 Hz, 1H), 8.16 (s, 1H), 7.64 (s, 1H), 7.57 (d, J=9.2 Hz, 1H), 7.55 (s, 1H), 7.18 (s, 1H), 7.02 (d, J=8.7 Hz, 1H), 5.36-5.17 (m, 1H), 5.15-4.95 (m, 2H), 4.38-4.29 (m, 1H), 3.59 (s, 3H), 3.48-3.35 (m, 2H), 3.14-2.97 (m, 2H), 2.69-2.55 (m, 2H), 2.44-2.30 (m, 1H), 2.10-1.99 (m, 1H).

### Example 104. Methyl-d₃ (3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate

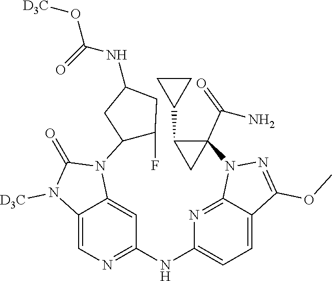 [SMILES: [2H]C([2H])([2H])OC(=O)NC1CC(F)C(n2c(=O)n(C([2H])([2H])[2H])c3cnc(Nc4ccc5c(OC)nn([C@@]6(C(N)=O)C[C@@H]6C6CC6)c5n4)cc32)C1]

The title compound was prepared according to the procedures described in Example 48, Step 1 to 5 and Example 102, Step 2, with methyl (3-(6-amino-3-(methyl-d₃)-2-oxo-2,3-dihydro-1H-imidazo[4,5-c]pyridin-1-yl)-4-fluorocyclopentyl)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate. LCMS calculated for C₂₈H₂₇D₆FN₉O₅(M+H)⁺: m/z=600.3; found 600.3.

### Example 105. Methyl-d₃ (3-(6-((1-((1R,2S)-2-carbamoyl-[1,1′-bi(cyclopropan)]-2-yl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate

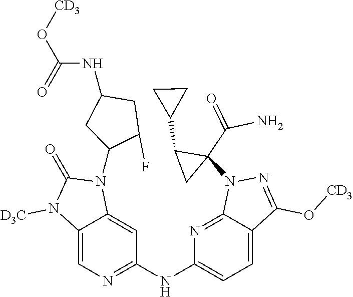 [SMILES: [2H]C([2H])([2H])OC(=O)NC1CC(F)C(n2c(=O)n(C([2H])([2H])[2H])c3cnc(Nc4ccc5c(OC([2H])([2H])[2H])nn([C@@]6(C(N)=O)C[C@@H]6C6CC6)c5n4)cc32)C1]

The title compound was prepared according to the procedures described in Example 48, Step 1 to 5, with methyl (3-(6-amino-3-(methyl-d₃)-2-oxo-2,3-dihydro-1H-imidazo[4,5-c]pyridin-1-yl)-4-fluorocyclopentyl)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 3 and methanol-d₄ replacing methanol in Step 4. LCMS calculated for C₂₈H₂₄D₉FN₉O₅ (M+H)⁺: m/z=603.3; found 603.3. ¹H NMR (500 MHz, DMSO) δ 11.07 (brs, 1H), 8.25 (s, 1H), 8.04 (d, J=8.5 Hz, 1H), 7.61 (s, 1H), 7.46 (d, J=7.3 Hz, 1H), 7.35 (s, 1H), 6.99-6.86 (m, 2H), 5.40-5.22 (m, 1H), 5.15-4.97 (m, 1H), 4.48-4.40 (m, 1H), 2.93-2.83 (m, 1H), 2.48-2.37 (m, 1H), 2.15-1.84 (m, 3H), 1.58-1.48 (m, 2H), 1.11-1.01 (m, 1H), 0.58-0.46 (m, 2H), 0.39-0.26 (m, 2H).

### Example 106. Methyl (3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate

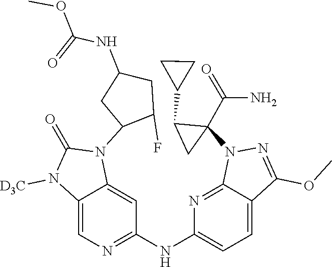 [SMILES: [2H]C([2H])([2H])n1c(=O)n(C2CC(NC(=O)OC)CC2F)c2cc(Nc3ccc4c(OC)nn([C@@]5(C(N)=O)C[C@@H]5C5CC5)c4n3)ncc21]

The title compound was prepared according to the procedures described in Example 48, Step 1 to 5, with methyl (3-(6-amino-3-(methyl-d₃)-2-oxo-2,3-dihydro-1H-imidazo[4,5-c]pyridin-1-yl)-4-fluorocyclopentyl)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate. LCMS calculated for C₂₈H₃₀D₃FN₉O₅(M+H)⁺: m/z=597.3; found 597.3. ¹H NMR (500 MHz, DMSO) δ 11.13 (brs, 1H), 8.25 (s, 1H), 8.03 (d, J=8.6 Hz, 1H), 7.64 (s, 1H), 7.47 (d, J=7.3 Hz, 1H), 7.36 (s, 1H), 6.99-6.86 (m, 2H), 5.43-5.22 (m, 1H), 5.15-4.97 (m, 1H), 4.48-4.40 (m, 1H), 4.00 (s, 3H), 3.55 (s, 3H), 2.93-2.83 (m, 1H), 2.48-2.35 (m, 1H), 2.13-1.88 (m, 3H), 1.58-1.48 (m, 2H), 1.11-1.01 (m, 1H), 0.58-0.46 (m, 2H), 0.39-0.27 (m, 2H).

### Example 108. Methyl ((1S,2S,4S)-4-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)-2-methoxycyclopentyl)carbamate

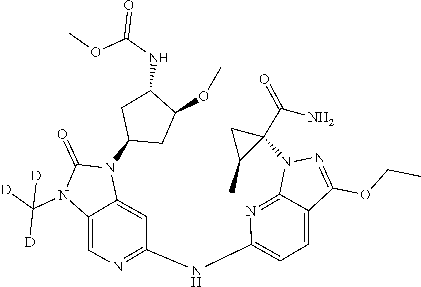 [SMILES: [2H]C([2H])([2H])n1c(=O)n([C@H]2C[C@H](NC(=O)OC)[C@@H](OC)C2)c2cc(Nc3ccc4c(OCC)nn([C@@]5(C(N)=O)C[C@@H]5C)c4n3)ncc21]

### Step 1. racemix-N-((1S,2S,4R)-4-Hydroxy-2-methoxycyclopentyl)-4-nitrobenzenesulfonamide

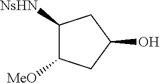

To a flask containing (1R,3r,5S)-6-((4-nitrophenyl)sulfonyl)-6-azabicyclo[3.1.0]hexan-3-ol (9.5 g, 33.4 mmol, Caine, D. et. al, Org. Lett. 2002, 4, 1923) and Cs₂CO₃ (16.33 g, 50.1 mmol) was added MeOH (15 mL) and THF (150 mL). The reaction was stirred at 50° C. for 15 min. The reaction mixture was concentrated in vacuo and the residue was basified with sat. NaHCO₃ and extracted with DCM (3×50 mL). The organic phases were combined, dried over Na₂SO₄ and concentrated in vacuo. The obtained crude product was purified by Biotage Isolera to give the desired product as a racemic mixture. ¹H NMR (500 MHz, DMSO) δ 8.42 (d, J=8.7 Hz, 2H), 8.21 (d, J=8.2 Hz, 1H), 8.07 (d, J=8.7 Hz, 2H), 4.71 (d, J=4.2 Hz, 1H), 4.02-3.91 (m, 1H), 3.54 (q, J=6.1 Hz, 1H), 3.44-3.33 (m, 1H), 2.97 (s, 3H), 2.10-1.89 (m, 1H), 1.76-1.54 (m, 2H), 1.32-1.07 (m, 1H).

### Step 2. racemix-N-((1S,2S,4S)-4-(1,3-Dioxoisoindolin-2-yl)-2-methoxycyclopentyl)-4-nitrobenzenesulfonamide

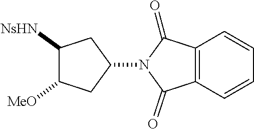

The title compound was prepared according to the procedures described in Intermediate A, Step 2, with racemix-N-((1S,2S,4R)-4-hydroxy-2-methoxycyclopentyl)-4-nitrobenzenesulfonamide (racemix from Step 1) replacing tert-butyl ((1R,3S)-3-hydroxycyclopentyl-3-d)carbamate. LCMS calculated for C₂₀H₂₀N₃O₇S (M+H)⁺: m/z=446.1; found: 446.2.

### Step 3. Methyl ((1R,2R,4R)-4-(1,3-dioxoisoindolin-2-yl)-2-methoxycyclopentyl)carbamate and Methyl ((1S,2S,4S)-4-(1,3-dioxoisoindolin-2-yl)-2-methoxycyclopentyl)carbamate

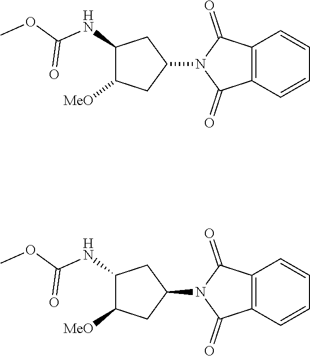

To a vial containing racemix-N-((1S,2S,4S)-4-(1,3-dioxoisoindolin-2-yl)-2-methoxycyclopentyl)-4-nitrobenzenesulfonamide (2.1 g, 4.7 mmol) and potassium carbonate (2.6 g, 18.9 mmol) in DMF (10 mL) was added 4-methoxybenzenethiol (1.0 g, 0.87 mL, 7.1 mmol). After stirring at r.t for 8 h, the reaction mixture was diluted with EtOAc (30 mL). The reaction mixture was washed with brine, dried over MgSO₄, and concentrated in vacuo. The obtained crude material was dissolved in CH₂Cl₂ (20 mL) and MeOH (1 mL) followed by the addition of DIPEA (1.9 g, 2.7 mL, 15.0 mmol). After cooling down to 0° C., methyl chloroformate (0.57 g, 0.46 mL, 6.0 mmol) was added dropwise. After stirring at 0° C. for 10 min., the reaction was quenched with MeOH (5 mL) and concentrated in vacuo. The crude material was purified by Biotage Isolera to give the desired product as a racemic mixture. The racemic mixture was further separated by chiral SFC (CHIRALPAK AY-H, 20 mm×250 mm, 5 μm column, eluting with 18% MeOH in CO₂, at flow rate of 75 mL/min over 9 min). The first peak was collected at 4.5 min as a single enantiomer. The second peak was collected at 6.2 min as a single enantiomer. LCMS calculated for peak 1 C₁₆H₁₉N₂O₅ (M+H)⁺: m/z=319.1; found 319.1. LCMS calculated for peak 2 C₁₆H₁₉N₂O₅ (M+H)⁺: m/z=319.1; found 319.1.

### Step 4. Methyl ((1S,2S,4S)-4-amino-2-methoxycyclopentyl)carbamate

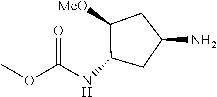

The title compound was prepared according to the procedures described in Intermediate A, Step 3, with methyl ((1S,2S,4S)-4-(1,3-dioxoisoindolin-2-yl)-2-methoxycyclopentyl)carbamate (peak 1 from Step 3) replacing tert-butyl ((1R,3R)-3-(1,3-dioxoisoindolin-2-yl)cyclopentyl-3-d)carbamate. LCMS calculated for C₈H₁₇N₂O₃ (M+H)⁺: m/z=189.1; found: 189.1.

### Step 5. Methyl ((1S,2S,4S)-4-(6-chloro-3-(methyl-d3)-2-oxo-2,3-dihydro-1H-imidazo[4,5-c]pyridin-1-yl)-2-methoxycyclopentyl)carbamate

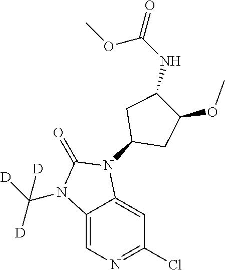 [SMILES: [2H]C([2H])([2H])n1c(=O)n([C@H]2C[C@H](NC(=O)OC)[C@@H](OC)C2)c2cc(Cl)ncc21]

The title compound was prepared according to the procedures described in Intermediate A, Step 5 to 8, with methyl ((1S,2S,4S)-4-amino-2-methoxycyclopentyl)carbamate replacing methyl ((1R,3R)-3-aminocyclopentyl-1-d)carbamate in Step 5. LCMS calculated for C₁₅H₁₇D₃ClN₄O₄(M+H)⁺: m/z=358.2; found: 358.2.

### Step 6. Ethyl (1S,2S)-1-(6-bromo-3-(2,2,2-trifluoroacetamido)-1H-pyrazolo[3,4-b]pyridin-1-yl)-2-methylcyclopropane-1-carboxylate

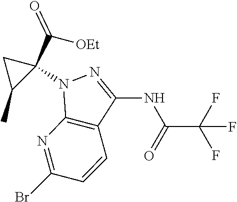 [SMILES: CCOC(=O)[C@]1(n2nc(NC(=O)C(F)(F)F)c3ccc(Br)nc32)C[C@@H]1C]

To a flask containing ethyl (1S,2S)-1-(3-amino-6-bromo-1H-pyrazolo[3,4-b]pyridin-1-yl)-2-methylcyclopropane-1-carboxylate (9.5 g, 28.0 mmol) in tetrahydrofuran (78 mL) and DIPEA (9.78 mL, 56.0 mmol) was slowly added 2,2,2-trifluoroacetic anhydride (6.62 mL, 47.6 mmol) at 0° C. The reaction mixture was stirred at r.t for 1 h. The mixture was concentrated in vacuo and purified by Biotage Isolera. LCMS calculated for C₁₅H₁₅BrF₃N₄O₃ (M+H)⁺: m/z=435.1; found: 435.1.

### Step 7. Ethyl (1S,2S)-1-(3-amino-6-((tert-butoxycarbonyl)amino)-1H-pyrazolo[3,4-b]pyridin-1-yl)-2-methylcyclopropane-1-carboxylate

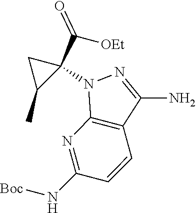 [SMILES: CCOC(=O)[C@]1(n2nc(N)c3ccc(NC(=O)OC(C)(C)C)nc32)C[C@@H]1C]

To a flask containing ethyl (1S,2S)-1-(6-bromo-3-(2,2,2-trifluoroacetamido)-1H-pyrazolo[3,4-b]pyridin-1-yl)-2-methylcyclopropane-1-carboxylate (9.9 g, 22.75 mmol), tert-butyl carbamate (5.60 g, 47.8 mmol), Xantphos (3.95 g, 6.82 mmol), Pd₂(dba)₃ (3.12 g, 3.41 mmol) and cesium carbonate (14.82 g, 45.5 mmol) was added 1,4-dioxane (134 mL) and heated at 90° C. for 1 h. The reaction mixture was cooled down and filtered off the solid. The liquor was concentrated, redissolved in DCM and water was added. The mixture was extracted with DCM, dried over Na₂SO₄, filtered and concentrated. To this crude material was added 7 N ammonia in MeOH (64 mL, 445 mmol). The mixture was heated to 60° C. for 8 h. The reaction mixture was cooled down and concentrated in vacuo. Sat. NaHCO₃ was added, and the mixture was extracted with DCM. The organic layers were combined, dried over Na₂SO₄, concentrated in vacuo and purified by Biotage Isolera. LCMS calculated for C₁₈H₂₆N₅O₄ (M+H)⁺: m/z=376.2; found: 376.2.

### Step 8. Ethyl (1S,2S)-1-(6-((tert-butoxycarbonyl)amino)-3-iodo-1H-pyrazolo[3,4-b]pyridin-1-yl)-2-methylcyclopropane-1-carboxylate

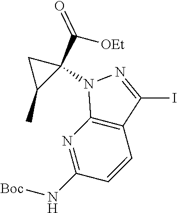 [SMILES: CCOC(=O)[C@]1(n2nc(I)c3ccc(NC(=O)OC(C)(C)C)nc32)C[C@@H]1C]

To a flask containing ethyl (1S,2S)-1-(3-amino-6-((tert-butoxycarbonyl)amino)-1H-pyrazolo[3,4-b]pyridin-1-yl)-2-methylcyclopropane-1-carboxylate (8.1 g, 21.58 mmol) in acetonitrile (144 mL) was added iodine (13.69 g, 53.9 mmol), potassium iodide (5.37 g, 32.4 mmol) and CuI (6.16 g, 32.4 mmol) at 0° C. After 5 min, tert-butylnitrite (10.26 mL, 86 mmol) was added in one portion and the mixture was stirred at r.t for 1.5 h. Then HI (4.27 mL, 32.4 mmol) was slowly added, and the mixture was stirred at r.t for 1 h. The reaction mixture was quenched with sat. Na₂S₂O₃ solution and extracted with DCM (3×50 mL). The organic phases were combined, dried over Na₂SO₄ and concentrated in vacuo. The obtained crude product was purified by Biotage Isolera. LCMS calculated for C₁₈H₂₄1N₄O₄(M+H)⁺: m/z=487.1; found: 487.1.

### Step 9. Ethyl (1S,2S)-1-(6-amino-3-ethoxy-1H-pyrazolo[3,4-b]pyridin-1-yl)-2-methylcyclopropane-1-carboxylate

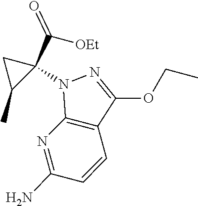 [SMILES: CCOC(=O)[C@]1(n2nc(OCC)c3ccc(N)nc32)C[C@@H]1C]

To a flask containing ethyl (1S,2S)-1-(6-((tert-butoxycarbonyl)amino)-3-iodo-1H-pyrazolo[3,4-b]pyridin-1-yl)-2-methylcyclopropane-1-carboxylate (2.5 g, 5.14 mmol), cesium carbonate (5.02 g, 15.42 mmol) and tBuBrettphos-Pd-G3 (0.351 g, 0.411 mmol) was added 1,4-dioxane (14.9 mL), EtOAc (0.75 mL) and ethanol (2 mL). The reaction mixture was degassed with N₂ flow for 5 min then heated at 100° C. for 2 h. The reaction mixture was cooled down to r.t, filtered off the solid, and concentrated. Then sat. NaHCO₃ was added and the mixture was extracted with EtOAc (3 times), dried over Na₂SO₄ and concentrated. The crude product was de-protected with TFA (20 mL) at r.t for 15 min. The TFA was removed in vacuo and basified with sat. NaHCO₃. The mixture was extracted with DCM, dried over Na₂SO₄, concentrated and purified by Biotage Isolera. LCMS calculated for C₁₅H₂₁N₄O₃ (M+H)⁺: m/z=305.2; found: 305.2.

### Step 10. (1S,2S)-1-(3-Ethoxy-6-((1-((1S,3S,4S)-3-methoxy-4-((methoxycarbonyl)amino)cyclopentyl)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid

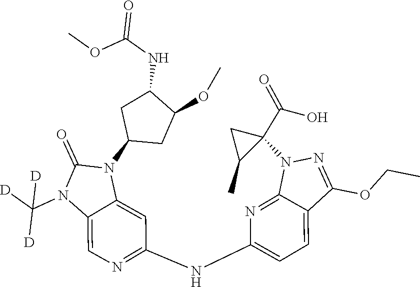 [SMILES: [2H]C([2H])([2H])n1c(=O)n([C@H]2C[C@H](NC(=O)OC)[C@@H](OC)C2)c2cc(Nc3ccc4c(OCC)nn([C@@]5(C(=O)O)C[C@@H]5C)c4n3)ncc21]

To a vial containing methyl ((1S,2S,4S)-4-(6-chloro-3-(methyl-d₃)-2-oxo-2,3-dihydro-1H-imidazo[4,5-c]pyridin-1-yl)-2-methoxycyclopentyl)carbamate (30 mg, 0.084 mmol), ethyl (1S,2S)-1-(6-amino-3-ethoxy-1H-pyrazolo[3,4-b]pyridin-1-yl)-2-methylcyclopropane-1-carboxylate (28 mg, 0.09 mmol) cesium carbonate (82 mg, 0.252 mmol), BrettPhos Pd G3 (23 mg, 0.025 mmol) was added dioxane (900 μL) under N₂ atmosphere. The reaction was stirred at 100° C. for 5 h. This was evacuated and backfilled with nitrogen 3 times. Subsequently 1,4-dioxane (2 mL) was added. The vial was sealed and heated to 100° C. for 2 h. Upon completion, the reaction was diluted with DCM, filtered through Celite and concentrated in vacuo. The crude residue was dissolved in a reaction vial containing THF (1 mL), MeOH (1 mL), and lithium hydroxide (1 mL, 1 M in H₂O), heated to 55° C., and allowed to stir for 4 h. Upon completion, the reaction was quenched with HCl (3 mL, 1 M in H₂O) and extracted with 3:1 chloroform:isopropanol. The organic layers were combined, dried over sodium sulfate, filtered, and the solvents were evaporated in vacuo. The crude material was used directly in the next reaction. LCMS calculated for C₂₈H₃₂D₃N₈O₇(M+H)⁺: m/z=598.2; found: 598.2.

### Step 11. Methyl ((1S,2S,4S)-4-(6-((1-((1S,2S)-1-carbamoyl-2-methylcyclopropyl)-3-ethoxy-1H-pyrazolo[3,4-b]pyridin-6-yl)amino)-3-(methyl-d3)-2-oxo-2,3-dihydro-1H-imidazo[4,5-c]pyridin-1-yl)-2-methoxycyclopentyl)carbamate

The title compound was prepared according to the procedures described in Example 14, Steps 4 with (1S,2S)-1-(3-ethoxy-6-((1-((1S,3S,4S)-3-methoxy-4-((methoxycarbonyl)amino)cyclopentyl)-3-(methyl-d₃)-2-oxo-2,3-dihydro-1H-imidazo[4,5-c]pyridin-6-yl)amino)-1H-pyrazolo[3,4-b]pyridin-1-yl)-2-methylcyclopropane-1-carboxylic acid replacing 3,3-difluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid. LCMS calculated for C₂₈H₃₃D₃N₉O₆(M+H)⁺: m/z=597.3; found 597.3. ¹H NMR (600 MHz, DMSO) δ 10.66 (s, 1H), 8.17 (s, 1H), 7.96 (s, 1H), 7.43 (s, 1H), 7.31 (s, 1H), 6.92 (d, J=8.7 Hz, 1H), 6.70 (s, 1H), 4.90 (s, 1H), 4.56-4.36 (m, 2H), 4.14 (s, 1H), 3.89-3.70 (m, 1H), 3.55 (s, 3H), 3.31 (s, 3H), 2.68-2.62 (m, 2H), 2.60-2.31 (m, 1H), 2.21-1.98 (m, 2H), 1.87-1.67 (m, 2H), 1.50-1.46 (m, 1H), 1.40 (t, J=7.0 Hz, 3H), 1.31 (d, J=6.3 Hz, 3H).

### Example A. JAK2 LanthaScreen JH1 Binding Assay

JAK2 JH1 binding assay utilizes catalytic domain (JH1, amino acids 826-1132) of human JAK2 expressed as N-terminal FLAG-tagged, biotinylated protein in a baculovirus expression system (Carna Biosciences, catalog #08-445-20N). The assay was conducted in black 384-well polystyrene plates in a final reaction volume of 20 μL. JAK2 JH1 (1.5 nM) was incubated with compounds (100 nL serially diluted in DMSO) in the presence of 50 nM fluorescent JAK2-JH1 tracer (Incyte Corp.) and 0.5 nM Streptavidin-Tb cryptate (Cisbio catalog #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 2 hours at 25° C., LanthaScreen signals were read on a PHERAstar FS plate reader (BMG LABTECH).

### Example B. JAK2 LanthaScreen JH2-WT Binding Assay

JAK2 JH2-WT binding assay utilizes pseudo-kinase domain (JH2, amino-acids 536-812 with 3 surface mutations W659A, W777A, F794H) of human Wild Type JAK2 expressed as C-terminal His-Avi-tagged, biotinylated protein in a baculovirus expression system (BPS Bioscience, Catalog #79463). The assay was conducted in black 384-well polystyrene plates in a final reaction volume of 20 μL. JAK2 JH2-WT (0.145 nM) was incubated with compounds (100 nL serially diluted in DMSO) in the presence of 50 nM Fluorescent JAK-JH2 Tracer (MedChem Express Catalog #HY-102055) and 0.25 nM Streptavidin-Tb cryptate (Cisbio catalog #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH).

### Example C. JAK2 LanthaScreen JH2-V617F Binding Assay

JAK2 JH2-V617F binding assay utilizes pseudo-kinase domain (JH2, amino-acids 536-812 with 3 surface mutations W659A, W777A, F794H) of human V617F mutant JAK2 expressed as C-terminal His-Avi-tagged, biotinylated protein in a baculovirus expression system (BPS Bioscience, Catalog #79498). The assay was conducted in black 384-well polystyrene plates in a final reaction volume of 20 μL. JAK2 JH2-V617F (0.26 nM) was incubated with compounds (100 nL serially diluted in DMSO) in the presence of 50 nM Fluorescent JAK-JH2 Tracer (MedChem Express Catalog #HY-102055) and 0.25 nM Streptavidin-Tb cryptate (Cisbio catalog #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH).

### Example D. JAK2 HTRF Enzyme Activity Assay

JAK2 enzyme activity assays utilize catalytic domain (JH1, amino acids 808-1132) of human JAK2 expressed as N-terminal His-tagged protein in a baculovirus expression system (BPS Bioscience, Catalog #40450). The assays was conducted in black 384-well polystyrene plates in a final reaction volume of 20 μL. JAK2 (0.015 nM) was incubated with compounds (100 nL serially diluted in DMSO) in the presence of ATP (30 μM or 1 mM) and 500 nM Biotin-labeled EQEDEPEGDYFEWLE (SEQ ID NO.: 1) peptide (BioSource International, custom synthesis) in assay buffer (50 mM Tris, pH=7.5, 10 mM MgCl₂, 0.01% Brij-35, 0.1% BSA, 1 mM EGTA, 5% Glycerol and 5 mM DTT) for 60 minutes at 25° C. The reactions were stopped by the addition of 10 μL of detection buffer (50 mM Tris, pH 7.8, 0.5 mg/mL BSA, 150 mM NaCl), supplemented with EDTA, LANCE Eu-W1024 anti-phosphotyrosine (PY20), (Revvity, catalog #AD0067) and Streptavidin SureLight APC (Revvity, catalog #AD0201), for a final concentration of 15 mM, 1.5 nM and 75 nM, respectively. HTRF signals were read after 30 minutes incubation at room temperature on a PHERAstar FS plate reader (BMG LABTECH).

### Example E. Biochemical Affinity Assay

The biochemical affinity of the compounds was measured using a TR-FRET assay in a 384-well plate, in which biotinylated JAK2-JH2 domain (for both wild type and the V617F mutant) and compounds of interest at 11 serially diluted concentrations were mixed together in the assay buffer that contains 50 mM Tris, 10 mM MgCl₂, 0.01% Brij-35, 0.1% BSA, 1 mM EGTA, 5 mM DTT, 5% glycerol, 0.5% DMSO, at pH 7.5. The reaction was incubated at room temperature to allow to reach equilibrium, followed by addition of the detection solution that contains Tb cryptate-labeled Streptavidin and a fluorescein-labelled tracer molecule. The plate was then read by BMG PHERAstar using LanthaScreen module.

### Example F. Permeability Assay

### Cell Culture

Caco-2 cells were grown at 37° C. in an atmosphere of 5% CO2 in DMEM growth medium supplemented with 10% (v/v) fetal bovine seru m, 1% (v/v) nonessential amino acids, penicillin (100 IU/mL), and streptomycin (100 μg/mL). Confluent cell monolayers were subcultured every 7 days by treatment with 0.05% trypsin containing 1 μM EDTA. Caco-2 cells were seeded in 96 well Millicell-96 plates. The seeding density for Caco-2 cells was 11,000 cells per well. Dulbecco's modified Eagle growth medium was replaced every other day after seeding. Cell monolayers were used for transport assays between 22 and 25 days.

### Permeability Assay

Cell culture medium was removed and replaced with HBSS. To measure the TEER, the HBSS was added into the donor compartment (apical side) and receiver compartment (basolateral side). The TEER was measured by using a REMS Autosampler (World Precision Instruments, Sarasota, FL) to ensure the integrity of the cell monolayers. Caco-2 cell monolayers with TEER values ≥300_cm2 were used for transport experiments. To determine the Papp in the absorptive direction (A-B), the compound solution dissolved in HBSS was added to the donor compartment (apical side), while the HBSS solution with 4% BSA was added to the receiver compartment (basolateral side). The apical volume was 0.075 mL and the basolateral volume was 0.25 mL. The incubation period was 120 minutes at 37° C. in an atmosphere of 5% CO2. At the end of the incubation period, samples from the donor and receiver sides were removed and an equal volume of acetonitrile was added for protein precipitation. The supernatants were collected after centrifugation (3000 rpm; Allegra X 14R Centrifuge, Beckman Coulter, Indianapolis, IN) for analysis.

### Data Analysis of Comparative Example A-C

Data was analyzed with IDBS XLfit and GraphPad Prism 5.0 software using a four parameter dose response curve to determine IC50 for each compound.

### Data Analysis of Compounds 1-108

Data analysis was done using Genedata Screener software (Version 21.0.3—Standard). Nonspecific binding in the presence of 2 mM ATP (for binding assays) or “no enzyme” control (for enzyme assay) was subtracted from raw data. Resulted data was normalized to percentage of vehicle (i.e., DMSO) negative control. The IC50 values were derived by fitting the data to a 4-parameter logistic equation producing a sigmoidal dose-response curve using Equation 1, where X is the logarithm of inhibitor concentration and Y is the response.

$$Y = Bottom + ( Top - Bottom ) / ( 1 + 10 \hat{} ( ( Log IC 50 - X ) \times Hill slope ) ) . \qquad Equation 1$$

This analysis was applied in the case of non-tight binding compounds such as Comparative Examples 1-6. For non-tight binding inhibitors, the relationship between IC50 and Ki^{app} can be defined as Equation 2.

$$IC 50 = {Ki}^{a p p} + 0.5 [ E ] . \qquad Equation 2$$

For non-tight binding compounds where Ki^{app} values are far greater than the enzyme concentration used in the assays, i.e. Ki^{app}>>[E], Equation 2 can be further approximated to Equation 3, where Ki^{app} is about equal to the IC50.

$$I C 50 ∼ K {i}^{a p p} . \qquad Equation 3$$

For tight binding compounds, such as Examples 1-108, automatic fitting to a tight binding model was applied in Genedata Screener software, and the apparent inhibition constants (Ki^{app}) were calculated using the quadratic Morrison equation (Equation 4), where concentrations of both the target enzyme and inhibitor compound are taken into consideration

$$\frac{{v}_{i}}{{v}_{0}} = 1 - \frac{( {[ E ]}_{T} + {[ I ]}_{T} + {K}_{i}^{app} ) - \sqrt{{( {[ E ]}_{T} + {[ I ]}_{T} + {K}_{i}^{app} )}^{2}} - {4 [ E ]}_{T}}{{2 [ E ]}_{T}} . \qquad Equation 4$$

A specific compound can be tight-binding inhibitor towards its intended target and a non-tight binding inhibitor for other proteins, and its selectivity can be calculated by dividing the IC50 of the non-tight binding proteins by the Ki^{app} of the tight-binding target (see e.g., Copeland, R. A. Evaluation of Enzyme Inhibitors in Drug Discovery: A Guide for Medicinal Chemists and Pharmacologists, 2nd Edition, 2013, 245-285).

The compounds of the disclosure and comparative examples were tested in one or more of the assays described in Examples A-I, and the resulting data are shown in Table A.

TABLE A

| | | | Selectivity | |
|---|---|---|---|---|
| | | JAK1 | (JAK1 | |
| | JH2 BIND | JH2 | JH2 BIND/ | |
| | V617F | BIND | JH2 BIND | CACO2 |
| | Ki^{app} | IC₅₀ | V617F | (×10⁻⁶ |
| Example | (nM) | (nM) | Ki^{app}) | cm/sec) |
| Comparative | ++ | [symbol] [symbol] [symbol] [symbol] | † | * |
| Example A | | | | |
| Comparative | ++ | [symbol] [symbol] [symbol] [symbol] | † | **** |
| Example B | | | | |
| Comparative | ++ | [symbol] [symbol] [symbol] | † | **** |
| Example C | | | | |
| 1 | ++ | [symbol] [symbol] [symbol] [symbol] [symbol] | ††† | *** |
| 2 | + | [symbol] [symbol] [symbol] | †† | **** |
| 3 | + | [symbol] [symbol] [symbol] [symbol] | ††† | *** |
| 4 | ++ | [symbol] [symbol] [symbol] [symbol] | †† | *** |
| 5 | + | [symbol] [symbol] [symbol] [symbol] | †††† | *** |
| 6 | + | [symbol] [symbol] [symbol] [symbol] | †† | *** |
| 7 | + | [symbol] [symbol] [symbol] [symbol] | †††† | |
| 8 | + | [symbol] [symbol] [symbol] | ††† | |
| 9 | + | [symbol] [symbol] [symbol] [symbol] | †††† | |
| 10 | + | [symbol] [symbol] [symbol] [symbol] | †††† | *** |
| 11 | + | [symbol] [symbol] [symbol] [symbol] | †††† | *** |
| 12 | + | [symbol] [symbol] [symbol] [symbol] | †† | *** |
| 13 | + | [symbol] [symbol] [symbol] | ††† | |
| 14 | + | [symbol] [symbol] [symbol] [symbol] | ††† | **** |
| 15 | + | [symbol] [symbol] [symbol] [symbol] | ††† | *** |
| 16 | + | [symbol] [symbol] [symbol] [symbol] | ††† | **** |
| 17 | + | [symbol] [symbol] [symbol] | †† | *** |
| 18 | + | [symbol] [symbol] [symbol] | †† | *** |
| 19 | + | [symbol] [symbol] [symbol] | †† | *** |
| 20 | + | [symbol] [symbol] [symbol] | †††† | |
| 21 | + | [symbol] [symbol] [symbol] [symbol] | †††† | **** |
| 22 | + | [symbol] [symbol] [symbol] [symbol] | †††† | **** |
| 23 | ++ | [symbol] [symbol] [symbol] [symbol] [symbol] | †††† | ** |
| 24 | + | [symbol] [symbol] [symbol] [symbol] | †††† | **** |
| 25 | + | [symbol] [symbol] [symbol] [symbol] | †††† | **** |
| Product 1 | | | | |
| 25 | ++ | [symbol] [symbol] [symbol] [symbol] [symbol] | ††† | * |
| Product 2 | | | | |
| 26 | + | [symbol] [symbol] [symbol] [symbol] | †††† | *** |
| Product 1 | | | | |
| 26 | ++ | [symbol] [symbol] [symbol] [symbol] [symbol] | †††† | ** |
| Product 2 | | | | |
| 27 | + | [symbol] [symbol] [symbol] [symbol] | †† | **** |
| 28 | + | [symbol] [symbol] [symbol] [symbol] | †† | *** |
| 29 | + | [symbol] [symbol] [symbol] [symbol] | ††† | *** |
| 30 | + | [symbol] [symbol] [symbol] [symbol] | †††† | **** |
| 31 | + | [symbol] [symbol] [symbol] | †† | **** |
| 32 | + | [symbol] [symbol] [symbol] [symbol] | †††† | **** |
| 33 | + | [symbol] [symbol] [symbol] [symbol] | †††† | *** |
| 34 | + | [symbol] [symbol] [symbol] [symbol] | †† | **** |
| 35 | + | [symbol] [symbol] [symbol] [symbol] | ††† | *** |
| 36 | + | [symbol] [symbol] [symbol] [symbol] | †† | **** |
| 37 | + | [symbol] [symbol] [symbol] [symbol] | ††† | **** |
| 38 | + | [symbol] [symbol] [symbol] [symbol] | †† | *** |
| 39 | + | [symbol] [symbol] [symbol] [symbol] | †† | |
| 40 | + | [symbol] [symbol] [symbol] [symbol] | ††† | **** |
| 41 | + | [symbol] [symbol] [symbol] [symbol] | ††† | **** |
| 42 | + | [symbol] [symbol] [symbol] [symbol] | †††† | **** |
| 43 | + | [symbol] [symbol] [symbol] [symbol] | ††† | **** |
| 44 | + | [symbol] [symbol] [symbol] [symbol] | ††† | *** |
| 45 | + | [symbol] [symbol] [symbol] [symbol] | ††† | **** |
| 46 | + | [symbol] [symbol] [symbol] [symbol] | ††† | **** |
| 47 | + | [symbol] [symbol] [symbol] | ††† | *** |
| 48 | + | [symbol] [symbol] [symbol] [symbol] | ††† | **** |
| 49 | + | [symbol] [symbol] [symbol] [symbol] | ††† | *** |
| 50 | + | [symbol] [symbol] [symbol] [symbol] | ††† | *** |
| 51 | + | [symbol] [symbol] [symbol] [symbol] | †† | **** |
| 52 | + | [symbol] [symbol] [symbol] [symbol] | ††† | **** |
| 53 | + | [symbol] [symbol] [symbol] [symbol] | ††† | **** |
| 54 | + | [symbol] [symbol] [symbol] [symbol] | †† | **** |
| 55 | + | [symbol] [symbol] [symbol] [symbol] | †† | **** |
| 56 | + | [symbol] [symbol] [symbol] [symbol] | ††† | *** |
| 57 | + | [symbol] [symbol] [symbol] [symbol] | †† | **** |
| 58 | + | [symbol] [symbol] [symbol] [symbol] | ††† | **** |
| 59 | + | [symbol] [symbol] [symbol] [symbol] | †† | **** |
| 60 | + | [symbol] [symbol] [symbol] [symbol] | ††† | *** |
| 61 | + | [symbol] [symbol] [symbol] [symbol] | †††† | *** |
| 62 | + | [symbol] [symbol] [symbol] [symbol] | ††† | *** |
| 63 | + | [symbol] [symbol] [symbol] [symbol] | ††† | *** |
| 64 | + | [symbol] [symbol] [symbol] [symbol] | †† | *** |
| 65 | + | [symbol] [symbol] [symbol] [symbol] | †† | *** |
| 66 | + | [symbol] [symbol] [symbol] | †† | **** |
| 67 | + | [symbol] [symbol] [symbol] [symbol] | †† | **** |
| 68 | + | [symbol] [symbol] [symbol] [symbol] | †††† | ** |
| 69 | + | [symbol] [symbol] [symbol] [symbol] | †††† | ** |
| 70 | + | [symbol] [symbol] [symbol] [symbol] | ††† | ** |
| 71 | + | [symbol] [symbol] [symbol] [symbol] | †† | **** |
| 72 | + | [symbol] [symbol] [symbol] [symbol] | †††† | **** |
| 73 | + | [symbol] [symbol] [symbol] [symbol] | ††† | **** |
| 74 | + | [symbol] [symbol] [symbol] [symbol] | †† | **** |
| 75 | + | [symbol] [symbol] [symbol] [symbol] | ††† | **** |
| 76 | + | [symbol] [symbol] [symbol] [symbol] | †† | *** |
| 77 | + | [symbol] [symbol] [symbol] [symbol] | †† | **** |
| 78 | + | [symbol] [symbol] [symbol] [symbol] | ††† | |
| 79 | + | [symbol] [symbol] [symbol] [symbol] | ††† | *** |
| 80 | + | [symbol] [symbol] [symbol] [symbol] | †† | *** |
| 81 | + | [symbol] [symbol] [symbol] [symbol] | †† | *** |
| 82 | ++ | [symbol] [symbol] [symbol] [symbol] | †† | *** |
| 83 | + | [symbol] [symbol] [symbol] [symbol] | ††† | *** |
| 84 | + | [symbol] [symbol] [symbol] [symbol] | †† | *** |
| 85 | + | [symbol] [symbol] [symbol] [symbol] | †† | *** |
| 86 | + | [symbol] [symbol] [symbol] [symbol] | †† | **** |
| 87 | + | [symbol] [symbol] [symbol] [symbol] | †† | **** |
| 88 | + | [symbol] [symbol] [symbol] [symbol] | ††† | *** |
| 89 | + | [symbol] [symbol] [symbol] [symbol] | †† | **** |
| 90 | + | [symbol] [symbol] [symbol] [symbol] | ††† | **** |
| 91 | + | [symbol] [symbol] [symbol] [symbol] | ††† | **** |
| 92 | + | [symbol] [symbol] [symbol] [symbol] | ††† | **** |
| 93 | + | [symbol] [symbol] [symbol] [symbol] | ††† | **** |
| 94 | + | [symbol] [symbol] [symbol] [symbol] | ††† | **** |
| 95 | + | [symbol] [symbol] [symbol] [symbol] | ††† | **** |
| 96 | + | [symbol] [symbol] [symbol] [symbol] | ††† | **** |
| 97 | + | [symbol] [symbol] [symbol] [symbol] | ††† | **** |
| 98 | + | [symbol] [symbol] [symbol] [symbol] | †† | **** |
| 99 | + | [symbol] [symbol] [symbol] [symbol] | ††† | *** |
| 100 | + | [symbol] [symbol] [symbol] [symbol] | †† | |
| 101 | + | [symbol] [symbol] [symbol] | ††† | |
| 102 | + | [symbol] [symbol] [symbol] [symbol] | †††† | *** |
| 103 | + | [symbol] [symbol] [symbol] [symbol] | †††† | *** |
| 104 | + | [symbol] [symbol] [symbol] [symbol] | ††† | **** |
| 105 | + | [symbol] [symbol] [symbol] [symbol] | †††† | **** |
| 106 | + | [symbol] [symbol] [symbol] [symbol] | †† | **** |
| 107 | + | [symbol] [symbol] [symbol] [symbol] | †† | *** |
| 108 | ++ | [symbol] [symbol] [symbol] [symbol] | †† | **** |

+ refers to Ki^{app} of ≤0.1 nM
++ refers to Ki^{app} of >0.1 nM to ≤1 nM
+++ refers to Ki^{app} of >1 nM to ≤10 nM
++++ refers to Ki^{app} of >10 nM to ≤100 nM
+++++ refers to Ki^{app} of >100 nM [symbol]
refers to IC₅₀ of ≤0.1 nM [symbol] [symbol]
refers to IC₅₀ of >0.1 nM to ≤1 nM [symbol] [symbol] [symbol]
refers to IC₅₀ of >1 nM to ≤10 nM [symbol] [symbol] [symbol] [symbol]
refers to IC₅₀ of >10 nM to ≤100 nM [symbol] [symbol] [symbol] [symbol] [symbol]
refers to IC₅₀ of >100 nM
† refers to selectivity of ≤100-fold
†† refers to selectivity of >100-fold to ≤500-fold
††† refers to selectivity of >500-fold to ≤1000-fold
†††† refers to selectivity of >1000-fold
* refers to ≤0.1
** refers to >0.1 to ≤1
*** refers to >1 to ≤10
**** refers to >10

### Example G. Cell Culture and STAT5 (Tyr694) Phosphorylation Cell Based Assay

Ba/F3 cells expressing human JAK2 V617F/EPOR (mouse JAK2 WT knocked out by CRISPR) are cultured in RPMI media with 10% FBS,1 μg/mL Puromycin, 1 mg/mL Geneticin (Thermo Fisher). Ba/F3 cells expressing human JAK2 WT/EPOR are cultured in RPMI media with 10% FBS,1 μg/mL Puromycin, 1 mg/mL Geneticin and 2 ng/mL EPO. 24 hours before the assay, the culture medium for JAK2 V617F/EPOR Ba/F3 cells are changed to RPMI with 10% FBS without antibiotic (assay medium 1). Culture medium for Ba/F3 cells expressing human JAK2 WT/EPOR are changed to RPMI with 10% FBS and 2 ng/mL EPO (R&D systems) without antibiotic (assay medium 2). 50 nL/well test compounds in DMSO are transferred to the 384 white low volume cell culture plate (Greiner Bio-one) by ECHO liquid handler (Labcyte). The cells are centrifuged, resuspended in the corresponding fresh assay medium and dispensed at 10 μL/well (6×10⁶ cells/mL) with 0.5% DMSO in the final assay. After the treated cells are incubated at 37° C., 5% CO₂ for 2 hours, 4 μL/well supplemented lysis buffer (100× blocking buffer diluted 25 fold in 4× lysis buffer, Perkin-Elmer) are added and incubated at room temperature for 60 min with gentle shaking on orbital shaker at 600 rpm. Phospho-STAT5 Cryptate antibody and Phospho-STAT5 d2 antibody (1:1 vol/vol, Perkin-Elmer) are premixed and diluted 20 fold within the detection buffer. 4 μL of the premixed antibody solution are added to each well followed with 16 hours incubation at room temperature. The product activity is determined by measuring the fluorescence at 620 nm and 665 nm on Pherastar microplate reader (BMG Labtech). A ratio is calculated (665/620 nm) for each well. Wells with DMSO serve as the positive controls and wells containing high concentration of control compound are used as negative controls. IC₅₀ determination is performed by fitting the curve of percent control activity versus the log of the compound concentration using the Genedata Screener software.

### Example H. JAK1 LanthaScreen JH2 Binding Assay

JAK1 JH2 binding assay utilizes pseudo-kinase domain (JH2, amino-acids 583-852) of human JAK1 expressed as C-terminal His-Avi-tagged, biotinylated protein in a baculovirus expression system (GenScript order #U6893EG310-7). The assay was conducted in black 384-well polystyrene plates in a final reaction volume of 20 μL. JAK1 JH2 (1.5 nM) was incubated with compounds (100 nL serially diluted in DMSO) in the presence of 5 nM Fluorescent JAK-JH2 Tracer (MedChem Express Catalog #HY-102055) and 0.25 nM Streptavidin-Tb cryptate (Cisbio catalog #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH).

### Example I. TYK2 LanthaScreen JH2 Binding Assay

TYK2 JH2 binding assay utilizes pseudo-kinase domain (JH2, amino-acids 575-869) of human TYK2 expressed as N-terminal His, C-terminal-Avi-tagged, biotinylated protein in a baculovirus expression system (GenScript order #U6893EG310-3). The assay was conducted in black 384-well polystyrene plates in a final reaction volume of 20 μL. TYK2 JH2 (3 nM) was incubated with compounds (100 nL serially diluted in DMSO) in the presence of 25 nM Fluorescent JAK-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH).

### Example J. In Vivo Models

Clonal hematopoiesis correlates with increased likelihood of developing several more severe diseases including hematologic malignancies such as MPN and AML, and cardiovascular diseases (CVD) such as atherosclerosis and venous thrombosis (see e.g., Weeks et al., NEJM Evid., 2023 May; Jaiswal et al., N. Engl. J. Med. 2017; Wolach et al., Sci. Transl. Med. 2018; Wang et al., Circ. Res. 2018 Nov. 9; 123(11)).

In vivo models consisting of mice inoculated with hematopoietic cells (human or murine) expressing the JAK2V617F oncogenic mutation (to establish clonal hematopoiesis) are used to demonstrate efficacy of JAK2V617F selective inhibitors against clonal hematopoiesis or its subsequent effects (e.g., MPN, CVD, etc.). A JAK2V617F selective inhibitor decreases the numbers of JAK2V617F expressing cells (allele burden) in mice and decreases the effects of these cells including aberrant cytokine production, inflammation, and clotting ability/Netosis. JAK2V617F allelic content is measurable by quantitative PCR methods. Inflammatory cytokine analyses are performed using Luminex, MSD, or OLink platforms. Netosis as a surrogate for clotting, is quantified ex vivo via commercially available citrullinated H3 ELISA kits, or by flow cytometry focused on neutrophils positively stained for DAPI and Sytox Orange. Clotting is also monitored by following megakaryocyte and platelet number, maturity, and activation, as well as by mouse tail vein coagulation assays.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula Ia: 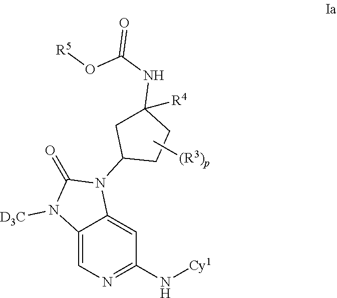
or a pharmaceutically acceptable salt thereof, wherein:
Cy¹ is selected from 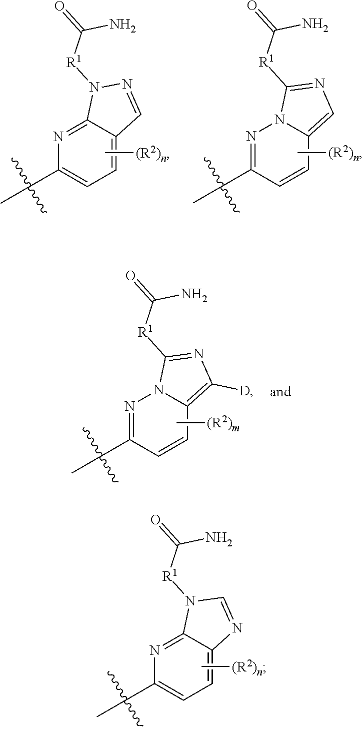
R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 R^{1A} substituents independently selected from halo, oxo, C₁₋₃ alkyl, C₁₋₃ haloalkyl, C₁₋₃ alkoxy, C₁₋₃ haloalkoxy, C₂₋₃ alkynyl, C₃₋₅ cycloalkyl, CN, OH, C(O)OH, and NH₂;
each R² is independently selected from halo, oxo, C₁₋₆ alkyl, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, CN, OR^{a21}, C(O)NR^{c21}R^{d21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)(═NR^{e21})R^{d21}, NR^{c21}S(O)₂NR^{c21}R^{d21}, S(O)₂R^{b21}, S(O)₂NR^{c21}R^{d21}, OS(O)(═NR^{e21})R^{b21}, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, or 4 independently selected R^{2A} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, or 4 independently selected R^{2A} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is independently selected from H, OH, halo, oxo, CN, C(O)OH, NH₂, NO₂, SF₅, C₁₋₆ alkyl, C₁₋₆alkoxy, C₁₋₆haloalkoxy, C₁₋₆haloalkyl, C₆₋₁₀ aryl, C₃₋₁₀cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, or 4 independently selected R^{M} substituents;
each R³ is independently selected from halo, oxo, C₁₋₃ alkyl, C₁₋₃ haloalkyl, C₁₋₃ alkoxy, C₁₋₃ haloalkoxy, CN, OH, C(O)OH, and NH₂;
R⁴ is H or D;
R⁵ is methyl or trideuteromethyl;
m is 0, 1, or 2;
n is 0, 1, 2, or 3;
p is 0, 1, or 2; and
each R^{M} is independently selected from OH, halo, oxo, CN, C(O)OH, NH₂, NO₂, SF₅, C₁₋₆ alkyl, C₁₋₆alkoxy, C₁₋₆haloalkoxy, C₁₋₆haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

2-7. (canceled)

8. The compound of claim 1, or a pharmaceutically acceptable salt thereof, wherein R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 R^{1A} substituents independently selected from halo, oxo, C₁₋₃ alkyl, C₁₋₃ haloalkyl, C₁₋₃ alkoxy, C₁₋₃ haloalkoxy, C₂₋₄ alkynyl, C₃₋₅ cycloalkyl, CN, OH, C(O)OH, and NH₂.

9-17. (canceled)

18. The compound of claim 1, or a pharmaceutically acceptable salt thereof, wherein n is 0, 1, or 2; m is 0; and p is 0 or 1.

19. (canceled)

20. The compound of claim 1, or a pharmaceutically acceptable salt thereof, wherein each R² is independently selected from C₁₋₆ alkyl, C₁₋₆ haloalkyl, C₃₋₁₀ cycloalkyl, 5-10 membered heteroaryl, 4-10 membered heterocycloalkyl, OR^{a21}, NR^{c21}R^{d21}, C(O)NR^{c21}R^{d21}, and NR^{c21}C(O)OR^{a21}, wherein the C₁₋₆ alkyl, C₃₋₁₀ cycloalkyl, 5-10 membered heteroaryl, and 4-10 membered heterocycloalkyl of R² are each optionally substituted with 1, 2, 3, or 4 independently selected R^{2A} substituents; and
each R^{a21}, R^{c21}, and R^{d21} is independently selected from H, C₁₋₆ alkyl, C₁₋₆ haloalkyl, C₂₋₆ alkenyl, and C₂₋₆ alkynyl.

21. (canceled)

22. The compound of claim 1, or a pharmaceutically acceptable salt thereof, wherein each R^{2A} is independently selected from OH, halo, C₁₋₆ alkyl, C₁₋₆ alkoxy, CN, and oxo.

23-26. (canceled)

27. The compound of claim 1, or a pharmaceutically acceptable salt thereof, wherein each R³ is independently selected from halo and C₁₋₃ alkoxy.

28. (canceled)

29. The compound of claim 1, or a pharmaceutically acceptable salt thereof, wherein R⁵ is trideuteromethyl.

30-33. (canceled)

34. The compound of claim 1, or a pharmaceutically acceptable salt thereof, wherein the compound of Formula Ia is a compound of Formula Ib, a compound of Formula Ic, a compound of Formula Id, a compound of Formula II, a compound of Formula III, a compound of Formula IV, a compound of Formula V, or a compound of Formula XIII: 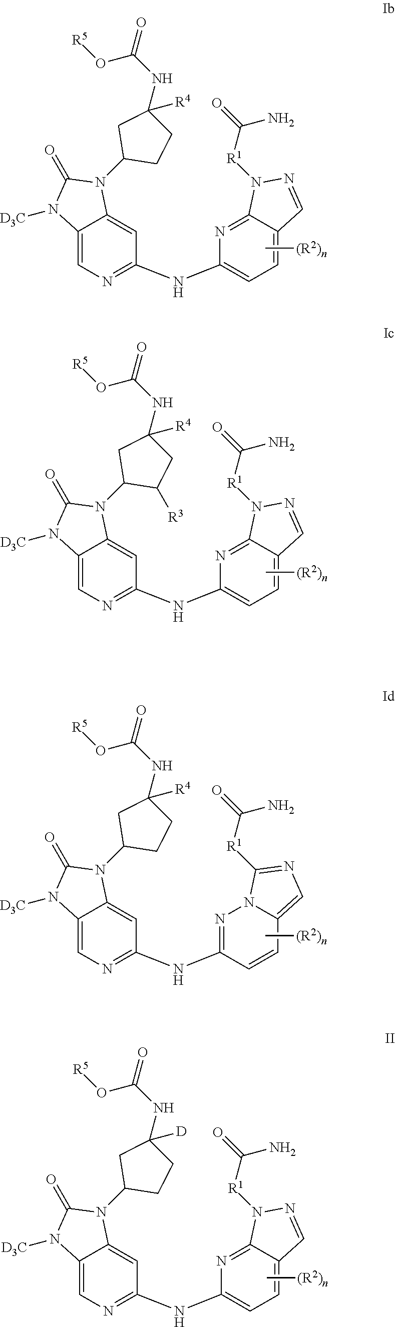 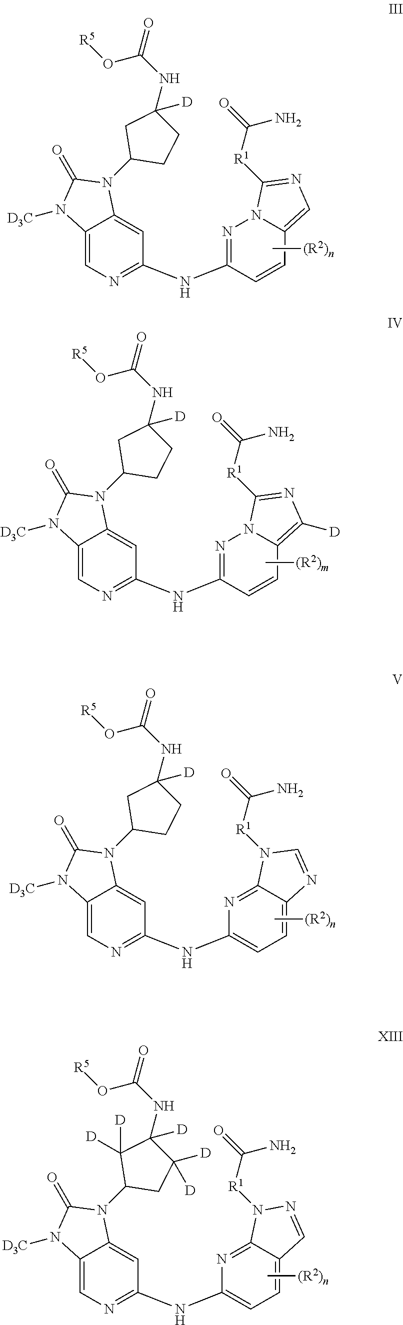
or a pharmaceutically acceptable salt thereof.

35-41. (canceled)

42. The compound of claim 1, which is selected from:
methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1,2,2,5,5-d₅)carbamate;
methyl ((1R,3R)-3-(6-((1-(1-carbamoylcyclobutyl)-4-(trans-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-methoxycyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-7-methyl-2-(trifluoromethyl)-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate; and
methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
or a pharmaceutically acceptable salt thereof.

43. The compound of claim 1, which is selected from:
methyl ((1R,3R)-3-(6-((1-((1S,2S)-1-carbamoyl-2-methylcyclopropyl)-3-ethyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(2-methoxypyridin-3-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(4-methoxypyridin-3-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2R)-1-carbamoyl-2-isopropylcyclopropyl)-3-((methoxycarbonyl)amino)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(trifluoromethyl)cyclopropyl)-3-((methoxycarbonyl)amino)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2R)-1-carbamoyl-2-isopropylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-morpholino-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(3-methyl-2-oxoimidazolidin-1-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(3,3-difluoroazetidin-1-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(2-oxopyrrolidin-1-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2R)-1-carbamoyl-2-ethynylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(trifluoromethyl)cyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(trifluoromethyl)cyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(trifluoromethyl)cyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(difluoromethyl)cyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-ethylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-ethylcyclopropyl)-3-(3,3-difluoroazetidin-1-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1R,2S)-2-carbamoyl-[1,1′-bi(cyclopropan)]-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-ethylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1R,2S)-2-carbamoyl-[1,1′-bi(cyclopropan)]-2-yl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-4-(1-methyl-1H-pyrazol-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-4-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(2,2,2-trifluoroethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3,4-dimethyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(methyl-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((1-((1S,2S)-1-carbamoyl-2-(difluoromethyl)cyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1R,2S)-1-carbamoyl-2-methylcyclopropyl)-5-(methyl-d₃)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1R,2S)-1-carbamoyl-2-methylcyclopropyl)-5-(trifluoromethyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl (3-(6-((1-((1S,2S)-1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate;
methyl (3-(6-((1-((1S,2S)-1-carbamoyl-2-ethylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate; and
methyl (3-(6-((1-((1S,2S)-1-carbamoyl-2-methylcyclopropyl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate;
or a pharmaceutically acceptable salt thereof.

44. The compound of claim 1, which is selected from:
methyl-d₃ ((1R,3R)-3-(6-((1-((1S,2S)-1-carbamoyl-2-methylcyclopropyl)-3-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(tetrahydro-2H-pyran-4-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl-d3 ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 ((1R,3R)-3-(6-((1-((1S,2S)-1-carbamoyl-2-methylcyclopropyl)-3-(dimethylamino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-(bis(methyl-d3)amino)-1-((1S,2S)-1-carbamoyl-2-methylcyclopropyl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(4-fluoro-2-azabicyclo[2.1.1]hexan-2-yl)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (1-((1S,3S)-1-carbamoyl-3-fluorocyclobutyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
ethyl (1-((1S,2S)-1-carbamoyl-2-methylcyclopropyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl (1-((1S,2S)-1-carbamoyl-2-methylcyclopropyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl (1-((1S,2S)-1-carbamoyl-2-methylcyclopropyl)-6-((1-((1R,3R)-3-(((methoxy-d3)carbonyl)amino)cyclopentyl)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl ((1R,3R)-3-(6-((1-((1S,2S)-1-carbamoyl-2-methylcyclopropyl)-3-((methoxycarbonyl)amino)-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl (1-((1S,2R)-1-carbamoyl-2-ethynylcyclopropyl)-6-((1-((1R,3R)-3-((methoxycarbonyl)amino)cyclopentyl-3-d)-3-(methyl-d3)-2-oxo-2,3-dihydro-1H-imidazo[4,5-c]pyridin-6-yl)amino)-1H-pyrazolo[3,4-b]pyridin-3-yl)(methyl)carbamate;
methyl ((1R,3R)-3-(6-((1-((1S,2R)-1-carbamoyl-2-ethynylcyclopropyl)-3-methyl-1H-pyrazolo[3,4-b]pyridin-6-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((1-((1R,2S)-2-carbamoyl-[1,1′-bi(cyclopropan)]-2-yl)-3-methyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl-d₃ ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate;
methyl-d3 ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-((1S,2S)-1-carbamoyl-2-methylcyclopropyl)-2-((methoxy-d3)methyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 ((1R,3R)-3-(6-((3-((1S,2S)-1-carbamoyl-2-methylcyclopropyl)-2-(methoxymethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-((1S,2S)-1-carbamoyl-2-methylcyclopropyl)-2-(methoxymethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl)carbamate;
methyl-d3 ((1R,3R)-3-(6-((3-((1S,2S)-1-carbamoyl-2-methylcyclopropyl)-2-((R)-1-(methoxy-d3)ethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl ((1R,3R)-3-(6-((3-((1S,2S)-1-carbamoyl-2-methylcyclopropyl)-2-((R)-1-methoxyethyl)-7-methyl-3H-imidazo[4,5-b]pyridin-5-yl)amino)-3-(methyl-d3)-2-oxo-2,3-dihydro-1H-imidazo[4,5-c]pyridin-1-yl)cyclopentyl-1-d)carbamate;
methyl-d3 ((1R,3R)-3-(6-((1-((1S,2S)-1-carbamoyl-2-methylcyclopropyl)-3-(methoxy-d3)-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d3 ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d3)-2-oxo-2,3-dihydro-1H-imidazo[4,5-c]pyridin-1-yl)cyclopentyl)carbamate;
methyl-d₃ ((1R,3R)-3-(6-((1-((1S,2S)-1-carbamoyl-2-methylcyclopropyl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl-d₃ ((1R,3R)-3-(6-((1-((1S,2S)-1-carbamoyl-2-methylcyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl-d₃ ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate;
methyl-d₃ ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate;
methyl-d₃ ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl)carbamate;
methyl-d₃ ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-methylcyclopropyl)-3-(1-cyanocyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1S,2S)-1-carbamoyl-2-(difluoromethyl)cyclopropyl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1R,2S)-1-carbamoyl-2-methylcyclopropyl)-4-methyl-5-(methyl-d₃)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1R,2S)-1-carbamoyl-2-methylcyclopropyl)-4-methyl-5-(methyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl-d₃ (4-(6-((1-((1s,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)-2-fluorocyclopentyl)carbamate;
methyl (4-(6-((1-((1s,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)-2-fluorocyclopentyl)carbamate;
methyl-d₃ (3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate;
methyl-d₃ (3-(6-((1-((1R,2S)-2-carbamoyl-[1,1′-bi(cyclopropan)]-2-yl)-3-(methoxy-d₃)-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate;
methyl (3-(6-((1-((1R,2S)-2-carbamoyl-[1,1′-bi(cyclopropan)]-2-yl)-3-methoxy-1H-pyrazolo[3,4-b]pyridin-6-yl)amino)-3-(methyl-d₃)-2-oxo-2,3-dihydro-1H-imidazo[4,5-c]pyridin-1-yl)-4-fluorocyclopentyl)carbamate; and
methyl ((1S,2S,4S)-4-(6-((1-((1S,2S)-1-carbamoyl-2-methylcyclopropyl)-3-ethoxy-1H-pyrazolo[3,4-b]pyridin-6-yl)amino)-3-(methyl-d₃)-2-oxo-2,3-dihydro-1H-imidazo[4,5-c]pyridin-1-yl)-2-methoxycyclopentyl)carbamate;
or a pharmaceutically acceptable salt thereof.

45. A compound, which is methyl ((1R,3R)-3-(6-((1-((1S,2S)-1-carbamoyl-2-methylcyclopropyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate, or a pharmaceutically acceptable salt thereof.

46. The compound of claim 45, which is methyl ((1R,3R)-3-(6-((1-((1S,2S)-1-carbamoyl-2-methylcyclopropyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate.

47. A compound, which is methyl ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate, or a pharmaceutically acceptable salt thereof.

48. The compound of claim 47, which is methyl ((1R,3R)-3-(6-((1-((1S,2S)-1-carbamoyl-2-methylcyclopropyl)-3-(dimethylcarbamoyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate.

49. (canceled)

50. A pharmaceutical composition, comprising a compound of claim 1, or a pharmaceutically acceptable salt thereof, and a pharmaceutically acceptable carrier.

51. (canceled)

52. A method of treating cancer in a patient in need thereof, the method comprising administering to the patient a therapeutically effective amount of a compound of claim 1, or a pharmaceutically acceptable salt thereof.

53-55. (canceled)

56. A method of treating a myeloproliferative disorder in a patient in need thereof, the method comprising administering to the patient a therapeutically effective amount of a compound of claim 1, or a pharmaceutically acceptable salt thereof.

57. (canceled)

58. A method of treating myelodysplastic syndrome in a patient in need thereof, the method comprising administering to the patient a therapeutically effective amount of a compound of claim 1, or a pharmaceutically acceptable salt thereof.

59. A method of treating clonal hematopoiesis of indeterminate potential in a patient in need thereof, comprising administering to the patient a therapeutically effective amount of a compound of claim 1, or a pharmaceutically acceptable salt thereof.


SEQUENCE LISTING (1 sequence)

SEQ ID NO: 1 - protein, 15 aa, synthetic construct
  REGION 1..15: note = Biotin-labeled peptide, custom synthesis
EQEDEPEGDYFEWLE